SEQUENCE LISTING (394 sequences)

SEQ ID NO: 1 - DNA, 2346 bp, Homo sapiens
  source 1..2346: mol_type = genomic DNA
atggctactcaagctgatttgatggagttggacatggccatggaaccagacagaaaagcggctgttagtcactggcagcaacagtcttacctggactctggaatccattctggtgccactaccacagctccttctctgagtggtaaaggcaatcctgaggaagaggatgtggatacctcccaagtcctgtatgagtgggaacagggattttctcagtccttcactcaagaacaagtagctgatattgatggacagtatgcaatgactcgagctcagagggtacgagctgctatgttccctgagacattagatgagggcatgcagatcccatctacacagtttgatgctgctcatcccactaatgtccagcgtttggctgaaccatcacagatgctgaaacatgcagttgtaaacttgattaactatcaagatgatgcagaacttgccacacgtgcaatccctgaactgacaaaactgctaaatgacgaggaccaggtggtggttaataaggctgcagttatggtccatcagctttctaaaaaggaagcttccagacacgctatcatgcgttctcctcagatggtgtctgctattgtacgtaccatgcagaatacaaatgatgtagaaacagctcgttgtaccgctgggaccttgcataacctttcccatcatcgtgagggcttactggccatctttaagtctggaggcattcctgccctggtgaaaatgcttggttcaccagtggattctgtgttgttttatgccattacaactctccacaaccttttattacatcaagaaggagctaaaatggcagtgcgtttagctggtgggctgcagaaaatggttgccttgctcaacaaaacaaatgttaaattcttggctattacgacagactgccttcaaattttagcttatggcaaccaagaaagcaagctcatcatactggctagtggtggaccccaagctttagtaaatataatgaggacctatacttacgaaaaactactgtggaccacaagcagagtgctgaaggtgctatctgtctgctctagtaataagccggctattgtagaagctggtggaatgcaagctttaggacttcacctgacagatccaagtcaacgtcttgttcagaactgtctttggactctcaggaatctttcagatgctgcaactaaacaggaagggatggaaggtctccttgggactcttgttcagcttctgggttcagatgatataaatgtggtcacctgtgcagctggaattctttctaacctcacttgcaataattataagaacaagatgatggtctgccaagtgggtggtatagaggctcttgtgcgtactgtccttcgggctggtgacagggaagacatcactgagcctgccatctgtgctcttcgtcatctgaccagccgacaccaagaagcagagatggcccagaatgcagttcgccttcactatggactaccagttgtggttaagctcttacacccaccatcccactggcctctgataaaggctactgttggattgattcgaaatcttgccctttgtcccgcaaatcatgcacctttgcgtgagcagggtgccattccacgactagttcagttgcttgttcgtgcacatcaggatacccagcgccgtacgtccatgggtgggacacagcagcaatttgtggagggggtccgcatggaagaaatagttgaaggttgtaccggagcccttcacatcctagctcgggatgttcacaaccgaattgttatcagaggactaaataccattccattgtttgtgcagctgctttattctcccattgaaaacatccaaagagtagctgcaggggtcctctgtgaacttgctcaggacaaggaagctgcagaagctattgaagctgagggagccacagctcctctgacagagttacttcactctaggaatgaaggtgtggcgacatatgcagctgctgttttgttccgaatgtctgaggacaagccacaagattacaagaaacggctttcagttgagctgaccagctctctcttcagaacagagccaatggcttggaatgagactgctgatcttggacttgatattggtgcccagggagaaccccttggatatcgccaggatgatcctagctatcgttcttttcactctggtggatatggccaggatgccttgggtatggaccccatgatggaacatgagatgggtggccaccaccctggtgctgactatccagttgatgggctgccagatctggggcatgcccaggacctcatggatgggctgcctccaggtgacagcaatcagctggcctggtttgatactgacctgtaa

SEQ ID NO: 2 - RNA, 2346 nt, Homo sapiens
  source 1..2346: mol_type = genomic RNA
atggctactcaagctgatttgatggagttggacatggccatggaaccagacagaaaagcggctgttagtcactggcagcaacagtcttacctggactctggaatccattctggtgccactaccacagctccttctctgagtggtaaaggcaatcctgaggaagaggatgtggatacctcccaagtcctgtatgagtgggaacagggattttctcagtccttcactcaagaacaagtagctgatattgatggacagtatgcaatgactcgagctcagagggtacgagctgctatgttccctgagacattagatgagggcatgcagatcccatctacacagtttgatgctgctcatcccactaatgtccagcgtttggctgaaccatcacagatgctgaaacatgcagttgtaaacttgattaactatcaagatgatgcagaacttgccacacgtgcaatccctgaactgacaaaactgctaaatgacgaggaccaggtggtggttaataaggctgcagttatggtccatcagctttctaaaaaggaagcttccagacacgctatcatgcgttctcctcagatggtgtctgctattgtacgtaccatgcagaatacaaatgatgtagaaacagctcgttgtaccgctgggaccttgcataacctttcccatcatcgtgagggcttactggccatctttaagtctggaggcattcctgccctggtgaaaatgcttggttcaccagtggattctgtgttgttttatgccattacaactctccacaaccttttattacatcaagaaggagctaaaatggcagtgcgtttagctggtgggctgcagaaaatggttgccttgctcaacaaaacaaatgttaaattcttggctattacgacagactgccttcaaattttagcttatggcaaccaagaaagcaagctcatcatactggctagtggtggaccccaagctttagtaaatataatgaggacctatacttacgaaaaactactgtggaccacaagcagagtgctgaaggtgctatctgtctgctctagtaataagccggctattgtagaagctggtggaatgcaagctttaggacttcacctgacagatccaagtcaacgtcttgttcagaactgtctttggactctcaggaatctttcagatgctgcaactaaacaggaagggatggaaggtctccttgggactcttgttcagcttctgggttcagatgatataaatgtggtcacctgtgcagctggaattctttctaacctcacttgcaataattataagaacaagatgatggtctgccaagtgggtggtatagaggctcttgtgcgtactgtccttcgggctggtgacagggaagacatcactgagcctgccatctgtgctcttcgtcatctgaccagccgacaccaagaagcagagatggcccagaatgcagttcgccttcactatggactaccagttgtggttaagctcttacacccaccatcccactggcctctgataaaggctactgttggattgattcgaaatcttgccctttgtcccgcaaatcatgcacctttgcgtgagcagggtgccattccacgactagttcagttgcttgttcgtgcacatcaggatacccagcgccgtacgtccatgggtgggacacagcagcaatttgtggagggggtccgcatggaagaaatagttgaaggttgtaccggagcccttcacatcctagctcgggatgttcacaaccgaattgttatcagaggactaaataccattccattgtttgtgcagctgctttattctcccattgaaaacatccaaagagtagctgcaggggtcctctgtgaacttgctcaggacaaggaagctgcagaagctattgaagctgagggagccacagctcctctgacagagttacttcactctaggaatgaaggtgtggcgacatatgcagctgctgttttgttccgaatgtctgaggacaagccacaagattacaagaaacggctttcagttgagctgaccagctctctcttcagaacagagccaatggcttggaatgagactgctgatcttggacttgatattggtgcccagggagaaccccttggatatcgccaggatgatcctagctatcgttcttttcactctggtggatatggccaggatgccttgggtatggaccccatgatggaacatgagatgggtggccaccaccctggtgctgactatccagttgatgggctgccagatctggggcatgcccaggacctcatggatgggctgcctccaggtgacagcaatcagctggcctggtttgatactgacctgtaa

SEQ ID NO: 3 - DNA, 2589 bp, Homo sapiens
  source 1..2589: mol_type = genomic DNA
atgaatatccaagagcagggtttccccttggacctcggagcaagtttcaccgaagatgctccccgacccccagtgcctggtgaggagggagaactggtgtccacagacccgaggcccgccagctacagtttctgctccgggaaaggtgttggcattaaaggtgagacttcgacggccactccgaggcgctcggatctggacctggggtatgagcctgagggcagtgcctcccccaccccaccatacttgaagtgggctgagtcactgcattccctgctggatgaccaagatgggataagcctgttcaggactttcctgaagcaggagggctgtgccgacttgctggacttctggtttgcctgcactggcttcaggaagctggagccctgtgactcgaacgaggagaagaggctgaagctggcgagagccatctaccgaaagtacattcttgataacaatggcatcgtgtcccggcagaccaagccagccaccaagagcttcataaagggctgcatcatgaagcagctgatcgatcctgccatgtttgaccaggcccagaccgaaatccaggccactatggaggaaaacacctatccctccttccttaagtctgatatttatttggaatatacgaggacaggctcggagagccccaaagtctgtagtgaccagagctctgggtcagggacagggaagggcatatctggatacctgccgaccttaaatgaagatgaggaatggaagtgtgaccaggacatggatgaggacgatggcagagacgctgctccccccggaagactccctcagaagctgctcctggagacagctgccccgagggtctcctccagtagacggtacagcgaaggcagagagttcaggtatggatcctggcgggagccagtcaacccctattatgtcaatgccggctatgccctggccccagccaccagtgccaacgacagcgagcagcagagcctgtccagcgatgcagacaccctgtccctcacggacagcagcgtggatgggatccccccatacaggatccgtaagcagcaccgcagggagatgcaggagagcgtgcaggtcaatgggcgggtgcccctacctcacattccccgcacgtaccgggtgccgaaggaggtccgcgtggagcctcagaagttcgcggaggagctcatccaccgcctggaggctgtgcagcgcacgcgggaggccgaggagaagctggaggagcggctgaagcgcgtgcgcatggaggaggaaggtgaggacggcgatccatcgtcagggcccccagggccgtgtcacaagctgcctcccgcccccgcttggcaccacttcccgccccgctgtgtggacatgggctgtgccgggctccgggatgcacacgaggagaaccctgagagcatcctggacgagcacgtacagcgtgtgctgaggacacctggccgccagtcgcctgggcctggccatcgctccccggacagtgggcacgtggccaagatgccagtggcactggggggtgccgcctcggggcacgggaagcacgtacccaagtcaggggcgaagctggacgcggccggcctgcaccaccaccgacacgtccaccaccacgtccaccacagcacagcccggcccaaggagcaggtggaggccgaggccacccgcagggcccagagcagcttcgcctggggcctggaaccacacagccatggggcaaggtcccgaggctactcagagagtgttggcgctgcccccaacgccagtgatggcctcgcccacagtgggaaggtgggcgtggcgtgcaaaagaaatgccaagaaggctgagtcggggaagagcgccagcaccgaggtgccaggtgcctcggaggatgcggagaagaaccagaaaatcatgcagtggatcattgagggggaaaaggagatcagcaggcaccgcaggaccggccacgggtcttcggggacgaggaagccacagccccatgagaactccagacccttgtcccttgagcacccctgggccggccctcagctccggacctccgtgcagccctcccacctcttcatccaagaccccaccatgccaccccacccagctcccaaccccctaacccagctggaggaggcgcgccgacgtctggaggaggaagaaaagagagccagccgagcaccctccaagcagaggtatgtgcaggaggttatgcggcggggacgcgcctgcgtcaggccagcgtgcgcgccggtgctgcacgtggtaccagccgtgtcggacatggagctctccgagacagagacaagatcgcagaggaaggtgggcggcgggagtgcccagccgtgtgacagcatcgttgtggcgtactacttctgcggggaacccatcccctaccgcaccctggtgaggggccgcgctgtcaccctgggccagttcaaggagctgctgaccaaaaagggcagctacagatactacttcaagaaagtgagcgacgagtttgactgtggggtggtgtttgaggaggttcgagaggacgaggccgtcctgcccgtctttgaggagaagatcatcggcaaagtggagaaggtggactga

SEQ ID NO: 4 - RNA, 2589 nt, Homo sapiens
  source 1..2589: mol_type = genomic RNA
atgaatatccaagagcagggtttccccttggacctcggagcaagtttcaccgaagatgctccccgacccccagtgcctggtgaggagggagaactggtgtccacagacccgaggcccgccagctacagtttctgctccgggaaaggtgttggcattaaaggtgagacttcgacggccactccgaggcgctcggatctggacctggggtatgagcctgagggcagtgcctcccccaccccaccatacttgaagtgggctgagtcactgcattccctgctggatgaccaagatgggataagcctgttcaggactttcctgaagcaggagggctgtgccgacttgctggacttctggtttgcctgcactggcttcaggaagctggagccctgtgactcgaacgaggagaagaggctgaagctggcgagagccatctaccgaaagtacattcttgataacaatggcatcgtgtcccggcagaccaagccagccaccaagagcttcataaagggctgcatcatgaagcagctgatcgatcctgccatgtttgaccaggcccagaccgaaatccaggccactatggaggaaaacacctatccctccttccttaagtctgatatttatttggaatatacgaggacaggctcggagagccccaaagtctgtagtgaccagagctctgggtcagggacagggaagggcatatctggatacctgccgaccttaaatgaagatgaggaatggaagtgtgaccaggacatggatgaggacgatggcagagacgctgctccccccggaagactccctcagaagctgctcctggagacagctgccccgagggtctcctccagtagacggtacagcgaaggcagagagttcaggtatggatcctggcgggagccagtcaacccctattatgtcaatgccggctatgccctggccccagccaccagtgccaacgacagcgagcagcagagcctgtccagcgatgcagacaccctgtccctcacggacagcagcgtggatgggatccccccatacaggatccgtaagcagcaccgcagggagatgcaggagagcgtgcaggtcaatgggcgggtgcccctacctcacattccccgcacgtaccgggtgccgaaggaggtccgcgtggagcctcagaagttcgcggaggagctcatccaccgcctggaggctgtgcagcgcacgcgggaggccgaggagaagctggaggagcggctgaagcgcgtgcgcatggaggaggaaggtgaggacggcgatccatcgtcagggcccccagggccgtgtcacaagctgcctcccgcccccgcttggcaccacttcccgccccgctgtgtggacatgggctgtgccgggctccgggatgcacacgaggagaaccctgagagcatcctggacgagcacgtacagcgtgtgctgaggacacctggccgccagtcgcctgggcctggccatcgctccccggacagtgggcacgtggccaagatgccagtggcactggggggtgccgcctcggggcacgggaagcacgtacccaagtcaggggcgaagctggacgcggccggcctgcaccaccaccgacacgtccaccaccacgtccaccacagcacagcccggcccaaggagcaggtggaggccgaggccacccgcagggcccagagcagcttcgcctggggcctggaaccacacagccatggggcaaggtcccgaggctactcagagagtgttggcgctgcccccaacgccagtgatggcctcgcccacagtgggaaggtgggcgtggcgtgcaaaagaaatgccaagaaggctgagtcggggaagagcgccagcaccgaggtgccaggtgcctcggaggatgcggagaagaaccagaaaatcatgcagtggatcattgagggggaaaaggagatcagcaggcaccgcaggaccggccacgggtcttcggggacgaggaagccacagccccatgagaactccagacccttgtcccttgagcacccctgggccggccctcagctccggacctccgtgcagccctcccacctcttcatccaagaccccaccatgccaccccacccagctcccaaccccctaacccagctggaggaggcgcgccgacgtctggaggaggaagaaaagagagccagccgagcaccctccaagcagaggtatgtgcaggaggttatgcggcggggacgcgcctgcgtcaggccagcgtgcgcgccggtgctgcacgtggtaccagccgtgtcggacatggagctctccgagacagagacaagatcgcagaggaaggtgggcggcgggagtgcccagccgtgtgacagcatcgttgtggcgtactacttctgcggggaacccatcccctaccgcaccctggtgaggggccgcgctgtcaccctgggccagttcaaggagctgctgaccaaaaagggcagctacagatactacttcaagaaagtgagcgacgagtttgactgtggggtggtgtttgaggaggttcgagaggacgaggccgtcctgcccgtctttgaggagaagatcatcggcaaagtggagaaggtggactga

SEQ ID NO: 5 - DNA, 2532 bp, Homo sapiens
  source 1..2532: mol_type = genomic DNA
atgagtagcgctatgttggtgacttgcctcccggaccccagcagcagcttccgtgaggatgccccgcggcccccagtgccaggggaagaaggggagaccccaccgtgtcagccaggggtgggcaagggccaggtcaccaaacccatgcctgtctcttccaacaccaggcggaacgaagatgggttgggggagccggaggggcgggcatctccggattcccctctgacccggtggaccaagtccttacactccttattgggcgatcaagacggtgcttacctgttccgaactttcctggagagggagaaatgcgtggataccttagacttctggtttgcctgcaatggattcaggcagatgaacctgaaggataccaaaactttacgagtagccaaagcgatctacaaaaggtacattgagaacaacagcattgtctccaagcagctgaagcctgccaccaagacctacataagagatggcatcaagaagcagcagattgattccatcatgtttgaccaggcgcagaccgagatccagtcggtgatggaggaaaatgcctaccagatgtttttgacttctgatatatacctcgaatatgtgaggagtgggggagaaaacacagcttacatgagtaatgggggactcgggagcctaaaggtcgtgtgtggctatctccccaccttgaatgaagaagaggagtggacttgtgccgacttcaagtgcaaactttcgccaaccgtggttggcttgtccagcaaaactctgagggccacggcgagtgtgaggtccacggaaactgttgacagtggatacaggtccttcaagaggagcgatcctgttaatccttatcacataggttctggctatgtctttgcaccagccaccagcgccaacgacagtgagatatccagtgatgcgctgacggatgattccatgtccatgacggacagcagtgtagatggaattcctccttatcgtgtgggcagtaagaaacagctccagagagaaatgcatcgcagtgtgaaggccaatggccaagtgtctctacctcatttcccgagaacccaccgcctgcccaaggagatgacccccgtggaacccgccacctttgcagctgagctgatctcgaggctggaaaagctgaagctggagttggagagccgccacagcctggaggagcgcctgcagcagatccgagaggatgaagagagagagggctccgagctcacactcaattcgcgggagggggcgcccacgcagcaccccctctccctactgccctccggcagctacgaggaagacccgcagacgatactggacgatcacctgtccagggtcctcaagacccctggctgccagtctccaggcgtaggccgctatagcccccgctcccgctccccggaccaccaccaccaccaccattcgcagtaccactccctgctcccgcccggtggcaagctgcctcccgcggccgcctcgccgggcgcctgccccctcctcgggggcaaaggctttgtgaccaagcagacgacgaagcatgtccaccaccactacatccaccaccatgccgtccccaagaccaaggaggagatcgaggcggaggccacgcagcgggtgcactgcttctgccctgggggcagcgagtattactgctactcgaaatgcaaaagccactccaaggctccggaaaccatgcccagcgagcagtttggcggcagcagaggcagtaccttgcccaaacgcaatgggaaaggcacggagccgggcctggccctgcccgccagggaaggaggggcccccggcggagctggggccctgcagcttccccgggaggaaggagacaggtcgcaggatgtctggcagtggatgctggagagtgagcggcagagcaagcccaagccccatagtgcccaaagcacaaaaaaggcctaccccttggagtctgcccgctcgtctccaggcgaacgagccagccggcaccatctgtgggggggcaacagcgggcacccccgcaccaccccccgtgcccacctgttcacccaggaccctgcgatgcctcccctgaccccacccaacacgctggctcagctggaggaggcctgtcgcaggctagctgaggtgtcgaagcccccaaagcagcggtgctgtgtggccagtcagcagagggacaggaatcattcggccactgttcagacgggagccacacccttctccaatccaagcctggctccagaagatcacaaagagccaaagaaactggcaggtgtccacgcgctccaggccagtgagttggttgtcacttactttttctgtggggaagaaattccataccggaggatgctgaaggctcagagcttgaccctgggccactttaaagagcagctcagcaaaaagggaaattataggtattacttcaaaaaagcaagcgatgagtttgcctgtggagcggtgtttgaggagatctgggaggatgagacggtgctcccgatgtatgaaggccggattctgggcaaagtggagcggatcgattga

SEQ ID NO: 6 - RNA, 2532 nt, Homo sapiens
  source 1..2532: mol_type = genomic RNA
atgagtagcgctatgttggtgacttgcctcccggaccccagcagcagcttccgtgaggatgccccgcggcccccagtgccaggggaagaaggggagaccccaccgtgtcagccaggggtgggcaagggccaggtcaccaaacccatgcctgtctcttccaacaccaggcggaacgaagatgggttgggggagccggaggggcgggcatctccggattcccctctgacccggtggaccaagtccttacactccttattgggcgatcaagacggtgcttacctgttccgaactttcctggagagggagaaatgcgtggataccttagacttctggtttgcctgcaatggattcaggcagatgaacctgaaggataccaaaactttacgagtagccaaagcgatctacaaaaggtacattgagaacaacagcattgtctccaagcagctgaagcctgccaccaagacctacataagagatggcatcaagaagcagcagattgattccatcatgtttgaccaggcgcagaccgagatccagtcggtgatggaggaaaatgcctaccagatgtttttgacttctgatatatacctcgaatatgtgaggagtgggggagaaaacacagcttacatgagtaatgggggactcgggagcctaaaggtcgtgtgtggctatctccccaccttgaatgaagaagaggagtggacttgtgccgacttcaagtgcaaactttcgccaaccgtggttggcttgtccagcaaaactctgagggccacggcgagtgtgaggtccacggaaactgttgacagtggatacaggtccttcaagaggagcgatcctgttaatccttatcacataggttctggctatgtctttgcaccagccaccagcgccaacgacagtgagatatccagtgatgcgctgacggatgattccatgtccatgacggacagcagtgtagatggaattcctccttatcgtgtgggcagtaagaaacagctccagagagaaatgcatcgcagtgtgaaggccaatggccaagtgtctctacctcatttcccgagaacccaccgcctgcccaaggagatgacccccgtggaacccgccacctttgcagctgagctgatctcgaggctggaaaagctgaagctggagttggagagccgccacagcctggaggagcgcctgcagcagatccgagaggatgaagagagagagggctccgagctcacactcaattcgcgggagggggcgcccacgcagcaccccctctccctactgccctccggcagctacgaggaagacccgcagacgatactggacgatcacctgtccagggtcctcaagacccctggctgccagtctccaggcgtaggccgctatagcccccgctcccgctccccggaccaccaccaccaccaccattcgcagtaccactccctgctcccgcccggtggcaagctgcctcccgcggccgcctcgccgggcgcctgccccctcctcgggggcaaaggctttgtgaccaagcagacgacgaagcatgtccaccaccactacatccaccaccatgccgtccccaagaccaaggaggagatcgaggcggaggccacgcagcgggtgcactgcttctgccctgggggcagcgagtattactgctactcgaaatgcaaaagccactccaaggctccggaaaccatgcccagcgagcagtttggcggcagcagaggcagtaccttgcccaaacgcaatgggaaaggcacggagccgggcctggccctgcccgccagggaaggaggggcccccggcggagctggggccctgcagcttccccgggaggaaggagacaggtcgcaggatgtctggcagtggatgctggagagtgagcggcagagcaagcccaagccccatagtgcccaaagcacaaaaaaggcctaccccttggagtctgcccgctcgtctccaggcgaacgagccagccggcaccatctgtgggggggcaacagcgggcacccccgcaccaccccccgtgcccacctgttcacccaggaccctgcgatgcctcccctgaccccacccaacacgctggctcagctggaggaggcctgtcgcaggctagctgaggtgtcgaagcccccaaagcagcggtgctgtgtggccagtcagcagagggacaggaatcattcggccactgttcagacgggagccacacccttctccaatccaagcctggctccagaagatcacaaagagccaaagaaactggcaggtgtccacgcgctccaggccagtgagttggttgtcacttactttttctgtggggaagaaattccataccggaggatgctgaaggctcagagcttgaccctgggccactttaaagagcagctcagcaaaaagggaaattataggtattacttcaaaaaagcaagcgatgagtttgcctgtggagcggtgtttgaggagatctgggaggatgagacggtgctcccgatgtatgaaggccggattctgggcaaagtggagcggatcgattga

SEQ ID NO: 7 - DNA, 8532 bp, Homo sapiens
  source 1..8532: mol_type = genomic DNA
atggctgcagcttcatatgatcagttgttaaagcaagttgaggcactgaagatggagaactcaaatcttcgacaagagctagaagataattccaatcatcttacaaaactggaaactgaggcatctaatatgaaggaagtacttaaacaactacaaggaagtattgaagatgaagctatggcttcttctggacagattgatttattagagcgtcttaaagagcttaacttagatagcagtaatttccctggagtaaaactgcggtcaaaaatgtccctccgttcttatggaagccgggaaggatctgtatcaagccgttctggagagtgcagtcctgttcctatgggttcatttccaagaagagggtttgtaaatggaagcagagaaagtactggatatttagaagaacttgagaaagagaggtcattgcttcttgctgatcttgacaaagaagaaaaggaaaaagactggtattacgctcaacttcagaatctcactaaaagaatagatagtcttcctttaactgaaaatttttccttacaaacagatatgaccagaaggcaattggaatatgaagcaaggcaaatcagagttgcgatggaagaacaactaggtacctgccaggatatggaaaaacgagcacagcgaagaatagccagaattcagcaaatcgaaaaggacatacttcgtatacgacagcttttacagtcccaagcaacagaagcagagaggtcatctcagaacaagcatgaaaccggctcacatgatgctgagcggcagaatgaaggtcaaggagtgggagaaatcaacatggcaacttctggtaatggtcagggttcaactacacgaatggaccatgaaacagccagtgttttgagttctagtagcacacactctgcacctcgaaggctgacaagtcatctgggaaccaaggtggaaatggtgtattcattgttgtcaatgcttggtactcatgataaggatgatatgtcgcgaactttgctagctatgtctagctcccaagacagctgtatatccatgcgacagtctggatgtcttcctctcctcatccagcttttacatggcaatgacaaagactctgtattgttgggaaattcccggggcagtaaagaggctcgggccagggccagtgcagcactccacaacatcattcactcacagcctgatgacaagagaggcaggcgtgaaatccgagtccttcatcttttggaacagatacgcgcttactgtgaaacctgttgggagtggcaggaagctcatgaaccaggcatggaccaggacaaaaatccaatgccagctcctgttgaacatcagatctgtcctgctgtgtgtgttctaatgaaactttcatttgatgaagagcatagacatgcaatgaatgaactagggggactacaggccattgcagaattattgcaagtggactgtgaaatgtatgggcttactaatgaccactacagtattacactaagacgatatgctggaatggctttgacaaacttgacttttggagatgtagccaacaaggctacgctatgctctatgaaaggctgcatgagagcacttgtggcccaactaaaatctgaaagtgaagacttacagcaggttattgcgagtgttttgaggaatttgtcttggcgagcagatgtaaatagtaaaaagacgttgcgagaagttggaagtgtgaaagcattgatggaatgtgctttagaagttaaaaaggaatcaaccctcaaaagcgtattgagtgccttatggaatttgtcagcacattgcactgagaataaagctgatatatgtgctgtagatggtgcacttgcatttttggttggcactcttacttaccggagccagacaaacactttagccattattgaaagtggaggtgggatattacggaatgtgtccagcttgatagctacaaatgaggaccacaggcaaatcctaagagagaacaactgtctacaaactttattacaacacttaaaatctcatagtttgacaatagtcagtaatgcatgtggaactttgtggaatctctcagcaagaaatcctaaagaccaggaagcattatgggacatgggggcagttagcatgctcaagaacctcattcattcaaagcacaaaatgattgctatgggaagtgctgcagctttaaggaatctcatggcaaataggcctgcgaagtacaaggatgccaatattatgtctcctggctcaagcttgccatctcttcatgttaggaaacaaaaagccctagaagcagaattagatgctcagcacttatcagaaacttttgacaatatagacaatttaagtcccaaggcatctcatcgtagtaagcagagacacaagcaaagtctctatggtgattatgtttttgacaccaatcgacatgatgataataggtcagacaattttaatactggcaacatgactgtcctttcaccatatttgaatactacagtgttacccagctcctcttcatcaagaggaagcttagatagttctcgttctgaaaaagatagaagtttggagagagaacgcggaattggtctaggcaactaccatccagcaacagaaaatccaggaacttcttcaaagcgaggtttgcagatctccaccactgcagcccagattgccaaagtcatggaagaagtgtcagccattcatacctctcaggaagacagaagttctgggtctaccactgaattacattgtgtgacagatgagagaaatgcacttagaagaagctctgctgcccatacacattcaaacacttacaatttcactaagtcggaaaattcaaataggacatgttctatgccttatgccaaattagaatacaagagatcttcaaatgatagtttaaatagtgtcagtagtagtgatggttatggtaaaagaggtcaaatgaaaccctcgattgaatcctattctgaagatgatgaaagtaagttttgcagttatggtcaatacccagccgacctagcccataaaatacatagtgcaaatcatatggatgataatgatggagaactagatacaccaataaattatagtcttaaatattcagatgagcagttgaactctggaaggcaaagtccttcacagaatgaaagatgggcaagacccaaacacataatagaagatgaaataaaacaaagtgagcaaagacaatcaaggaatcaaagtacaacttatcctgtttatactgagagcactgatgataaacacctcaagttccaaccacattttggacagcaggaatgtgtttctccatacaggtcacggggagccaatggttcagaaacaaatcgagtgggttctaatcatggaattaatcaaaatgtaagccagtctttgtgtcaagaagatgactatgaagatgataagcctaccaattatagtgaacgttactctgaagaagaacagcatgaagaagaagagagaccaacaaattatagcataaaatataatgaagagaaacgtcatgtggatcagcctattgattatagtttaaaatatgccacagatattccttcatcacagaaacagtcattttcattctcaaagagttcatctggacaaagcagtaaaaccgaacatatgtcttcaagcagtgagaatacgtccacaccttcatctaatgccaagaggcagaatcagctccatccaagttctgcacagagtagaagtggtcagcctcaaaaggctgccacttgcaaagtttcttctattaaccaagaaacaatacagacttattgtgtagaagatactccaatatgtttttcaagatgtagttcattatcatctttgtcatcagctgaagatgaaataggatgtaatcagacgacacaggaagcagattctgctaataccctgcaaatagcagaaataaaagaaaagattggaactaggtcagctgaagatcctgtgagcgaagttccagcagtgtcacagcaccctagaaccaaatccagcagactgcagggttctagtttatcttcagaatcagccaggcacaaagctgttgaattttcttcaggagcgaaatctccctccaaaagtggtgctcagacacccaaaagtccacctgaacactatgttcaggagaccccactcatgtttagcagatgtacttctgtcagttcacttgatagttttgagagtcgttcgattgccagctccgttcagagtgaaccatgcagtggaatggtaagtggcattataagccccagtgatcttccagatagccctggacaaaccatgccaccaagcagaagtaaaacacctccaccacctcctcaaacagctcaaaccaagcgagaagtacctaaaaataaagcacctactgctgaaaagagagagagtggacctaagcaagctgcagtaaatgctgcagttcagagggtccaggttcttccagatgctgatactttattacattttgccacggaaagtactccagatggattttcttgttcatccagcctgagtgctctgagcctcgatgagccatttatacagaaagatgtggaattaagaataatgcctccagttcaggaaaatgacaatgggaatgaaacagaatcagagcagcctaaagaatcaaatgaaaaccaagagaaagaggcagaaaaaactattgattctgaaaaggacctattagatgattcagatgatgatgatattgaaatactagaagaatgtattatttctgccatgccaacaaagtcatcacgtaaagcaaaaaagccagcccagactgcttcaaaattacctccacctgtggcaaggaaaccaagtcagctgcctgtgtacaaacttctaccatcacaaaacaggttgcaaccccaaaagcatgttagttttacaccgggggatgatatgccacgggtgtattgtgttgaagggacacctataaacttttccacagctacatctctaagtgatctaacaatcgaatcccctccaaatgagttagctgctggagaaggagttagaggaggggcacagtcaggtgaatttgaaaaacgagataccattcctacagaaggcagaagtacagatgaggctcaaggaggaaaaacctcatctgtaaccatacctgaattggatgacaataaagcagaggaaggtgatattcttgcagaatgcattaattctgctatgcccaaagggaaaagtcacaagcctttccgtgtgaaaaagataatggaccaggtccagcaagcatctgcgtcttcttctgcacccaacaaaaatcagttagatggtaagaaaaagaaaccaacttcaccagtaaaacctataccacaaaatactgaatataggacacgtgtaagaaaaaatgcagactcaaaaaataatttaaatgctgagagagttttctcagacaacaaagattcaaagaaacagaatttgaaaaataattccaaggtcttcaatgataagctcccaaataatgaagatagagtcagaggaagttttgcttttgattcacctcatcattacacgcctattgaaggaactccttactgtttttcacgaaatgattctttgagttctctagattttgatgatgatgatgttgacctttccagggaaaaggctgaattaagaaaggcaaaagaaaataaggaatcagaggctaaagttaccagccacacagaactaacctccaaccaacaatcagctaataagacacaagctattgcaaagcagccaataaatcgaggtcagcctaaacccatacttcagaaacaatccacttttccccagtcatccaaagacataccagacagaggggcagcaactgatgaaaagttacagaattttgctattgaaaatactccggtttgcttttctcataattcctctctgagttctctcagtgacattgaccaagaaaacaacaataaagaaaatgaacctatcaaagagactgagccccctgactcacagggagaaccaagtaaacctcaagcatcaggctatgctcctaaatcatttcatgttgaagataccccagtttgtttctcaagaaacagttctctcagttctcttagtattgactctgaagatgacctgttgcaggaatgtataagctccgcaatgccaaaaaagaaaaagccttcaagactcaagggtgataatgaaaaacatagtcccagaaatatgggtggcatattaggtgaagatctgacacttgatttgaaagatatacagagaccagattcagaacatggtctatcccctgattcagaaaattttgattggaaagctattcaggaaggtgcaaattccatagtaagtagtttacatcaagctgctgctgctgcatgtttatctagacaagcttcgtctgattcagattccatcctttccctgaaatcaggaatctctctgggatcaccatttcatcttacacctgatcaagaagaaaaaccctttacaagtaataaaggcccacgaattctaaaaccaggggagaaaagtacattggaaactaaaaagatagaatctgaaagtaaaggaatcaaaggaggaaaaaaagtttataaaagtttgattactggaaaagttcgatctaattcagaaatttcaggccaaatgaaacagccccttcaagcaaacatgccttcaatctctcgaggcaggacaatgattcatattccaggagttcgaaatagctcctcaagtacaagtcctgtttctaaaaaaggcccaccccttaagactccagcctccaaaagccctagtgaaggtcaaacagccaccacttctcctagaggagccaagccatctgtgaaatcagaattaagccctgttgccaggcagacatcccaaataggtgggtcaagtaaagcaccttctagatcaggatctagagattcgaccccttcaagacctgcccagcaaccattaagtagacctatacagtctcctggccgaaactcaatttcccctggtagaaatggaataagtcctcctaacaaattatctcaacttccaaggacatcatcccctagtactgcttcaactaagtcctcaggttctggaaaaatgtcatatacatctccaggtagacagatgagccaacagaaccttaccaaacaaacaggtttatccaagaatgccagtagtattccaagaagtgagtctgcctccaaaggactaaatcagatgaataatggtaatggagccaataaaaaggtagaactttctagaatgtcttcaactaaatcaagtggaagtgaatctgatagatcagaaagacctgtattagtacgccagtcaactttcatcaaagaagctccaagcccaaccttaagaagaaaattggaggaatctgcttcatttgaatctctttctccatcatctagaccagcttctcccactaggtcccaggcacaaactccagttttaagtccttcccttcctgatatgtctctatccacacattcgtctgttcaggctggtggatggcgaaaactcccacctaatctcagtcccactatagagtataatgatggaagaccagcaaagcgccatgatattgcacggtctcattctgaaagtccttctagacttccaatcaataggtcaggaacctggaaacgtgagcacagcaaacattcatcatcccttcctcgagtaagcacttggagaagaactggaagttcatcttcaattctttctgcttcatcagaatccagtgaaaaagcaaaaagtgaggatgaaaaacatgtgaactctatttcaggaaccaaacaaagtaaagaaaaccaagtatccgcaaaaggaacatggagaaaaataaaagaaaatgaattttctcccacaaatagtacttctcagaccgtttcctcaggtgctacaaatggtgctgaatcaaagactctaatttatcaaatggcacctgctgtttctaaaacagaggatgtttgggtgagaattgaggactgtcccattaacaatcctagatctggaagatctcccacaggtaatactcccccggtgattgacagtgtttcagaaaaggcaaatccaaacattaaagattcaaaagataatcaggcaaaacaaaatgtgggtaatggcagtgttcccatgcgtaccgtgggtttggaaaatcgcctgaactcctttattcaggtggatgcccctgaccaaaaaggaactgagataaaaccaggacaaaataatcctgtccctgtatcagagactaatgaaagttctatagtggaacgtaccccattcagttctagcagctcaagcaaacacagttcacctagtgggactgttgctgccagagtgactccttttaattacaacccaagccctaggaaaagcagcgcagatagcacttcagctcggccatctcagatcccaactccagtgaataacaacacaaagaagcgagattccaaaactgacagcacagaatccagtggaacccaaagtcctaagcgccattctgggtcttaccttgtgacatctgtttaa

SEQ ID NO: 8 - RNA, 8532 nt, Homo sapiens
  source 1..8532: mol_type = genomic RNA
atggctgcagcttcatatgatcagttgttaaagcaagttgaggcactgaagatggagaactcaaatcttcgacaagagctagaagataattccaatcatcttacaaaactggaaactgaggcatctaatatgaaggaagtacttaaacaactacaaggaagtattgaagatgaagctatggcttcttctggacagattgatttattagagcgtcttaaagagcttaacttagatagcagtaatttccctggagtaaaactgcggtcaaaaatgtccctccgttcttatggaagccgggaaggatctgtatcaagccgttctggagagtgcagtcctgttcctatgggttcatttccaagaagagggtttgtaaatggaagcagagaaagtactggatatttagaagaacttgagaaagagaggtcattgcttcttgctgatcttgacaaagaagaaaaggaaaaagactggtattacgctcaacttcagaatctcactaaaagaatagatagtcttcctttaactgaaaatttttccttacaaacagatatgaccagaaggcaattggaatatgaagcaaggcaaatcagagttgcgatggaagaacaactaggtacctgccaggatatggaaaaacgagcacagcgaagaatagccagaattcagcaaatcgaaaaggacatacttcgtatacgacagcttttacagtcccaagcaacagaagcagagaggtcatctcagaacaagcatgaaaccggctcacatgatgctgagcggcagaatgaaggtcaaggagtgggagaaatcaacatggcaacttctggtaatggtcagggttcaactacacgaatggaccatgaaacagccagtgttttgagttctagtagcacacactctgcacctcgaaggctgacaagtcatctgggaaccaaggtggaaatggtgtattcattgttgtcaatgcttggtactcatgataaggatgatatgtcgcgaactttgctagctatgtctagctcccaagacagctgtatatccatgcgacagtctggatgtcttcctctcctcatccagcttttacatggcaatgacaaagactctgtattgttgggaaattcccggggcagtaaagaggctcgggccagggccagtgcagcactccacaacatcattcactcacagcctgatgacaagagaggcaggcgtgaaatccgagtccttcatcttttggaacagatacgcgcttactgtgaaacctgttgggagtggcaggaagctcatgaaccaggcatggaccaggacaaaaatccaatgccagctcctgttgaacatcagatctgtcctgctgtgtgtgttctaatgaaactttcatttgatgaagagcatagacatgcaatgaatgaactagggggactacaggccattgcagaattattgcaagtggactgtgaaatgtatgggcttactaatgaccactacagtattacactaagacgatatgctggaatggctttgacaaacttgacttttggagatgtagccaacaaggctacgctatgctctatgaaaggctgcatgagagcacttgtggcccaactaaaatctgaaagtgaagacttacagcaggttattgcgagtgttttgaggaatttgtcttggcgagcagatgtaaatagtaaaaagacgttgcgagaagttggaagtgtgaaagcattgatggaatgtgctttagaagttaaaaaggaatcaaccctcaaaagcgtattgagtgccttatggaatttgtcagcacattgcactgagaataaagctgatatatgtgctgtagatggtgcacttgcatttttggttggcactcttacttaccggagccagacaaacactttagccattattgaaagtggaggtgggatattacggaatgtgtccagcttgatagctacaaatgaggaccacaggcaaatcctaagagagaacaactgtctacaaactttattacaacacttaaaatctcatagtttgacaatagtcagtaatgcatgtggaactttgtggaatctctcagcaagaaatcctaaagaccaggaagcattatgggacatgggggcagttagcatgctcaagaacctcattcattcaaagcacaaaatgattgctatgggaagtgctgcagctttaaggaatctcatggcaaataggcctgcgaagtacaaggatgccaatattatgtctcctggctcaagcttgccatctcttcatgttaggaaacaaaaagccctagaagcagaattagatgctcagcacttatcagaaacttttgacaatatagacaatttaagtcccaaggcatctcatcgtagtaagcagagacacaagcaaagtctctatggtgattatgtttttgacaccaatcgacatgatgataataggtcagacaattttaatactggcaacatgactgtcctttcaccatatttgaatactacagtgttacccagctcctcttcatcaagaggaagcttagatagttctcgttctgaaaaagatagaagtttggagagagaacgcggaattggtctaggcaactaccatccagcaacagaaaatccaggaacttcttcaaagcgaggtttgcagatctccaccactgcagcccagattgccaaagtcatggaagaagtgtcagccattcatacctctcaggaagacagaagttctgggtctaccactgaattacattgtgtgacagatgagagaaatgcacttagaagaagctctgctgcccatacacattcaaacacttacaatttcactaagtcggaaaattcaaataggacatgttctatgccttatgccaaattagaatacaagagatcttcaaatgatagtttaaatagtgtcagtagtagtgatggttatggtaaaagaggtcaaatgaaaccctcgattgaatcctattctgaagatgatgaaagtaagttttgcagttatggtcaatacccagccgacctagcccataaaatacatagtgcaaatcatatggatgataatgatggagaactagatacaccaataaattatagtcttaaatattcagatgagcagttgaactctggaaggcaaagtccttcacagaatgaaagatgggcaagacccaaacacataatagaagatgaaataaaacaaagtgagcaaagacaatcaaggaatcaaagtacaacttatcctgtttatactgagagcactgatgataaacacctcaagttccaaccacattttggacagcaggaatgtgtttctccatacaggtcacggggagccaatggttcagaaacaaatcgagtgggttctaatcatggaattaatcaaaatgtaagccagtctttgtgtcaagaagatgactatgaagatgataagcctaccaattatagtgaacgttactctgaagaagaacagcatgaagaagaagagagaccaacaaattatagcataaaatataatgaagagaaacgtcatgtggatcagcctattgattatagtttaaaatatgccacagatattccttcatcacagaaacagtcattttcattctcaaagagttcatctggacaaagcagtaaaaccgaacatatgtcttcaagcagtgagaatacgtccacaccttcatctaatgccaagaggcagaatcagctccatccaagttctgcacagagtagaagtggtcagcctcaaaaggctgccacttgcaaagtttcttctattaaccaagaaacaatacagacttattgtgtagaagatactccaatatgtttttcaagatgtagttcattatcatctttgtcatcagctgaagatgaaataggatgtaatcagacgacacaggaagcagattctgctaataccctgcaaatagcagaaataaaagaaaagattggaactaggtcagctgaagatcctgtgagcgaagttccagcagtgtcacagcaccctagaaccaaatccagcagactgcagggttctagtttatcttcagaatcagccaggcacaaagctgttgaattttcttcaggagcgaaatctccctccaaaagtggtgctcagacacccaaaagtccacctgaacactatgttcaggagaccccactcatgtttagcagatgtacttctgtcagttcacttgatagttttgagagtcgttcgattgccagctccgttcagagtgaaccatgcagtggaatggtaagtggcattataagccccagtgatcttccagatagccctggacaaaccatgccaccaagcagaagtaaaacacctccaccacctcctcaaacagctcaaaccaagcgagaagtacctaaaaataaagcacctactgctgaaaagagagagagtggacctaagcaagctgcagtaaatgctgcagttcagagggtccaggttcttccagatgctgatactttattacattttgccacggaaagtactccagatggattttcttgttcatccagcctgagtgctctgagcctcgatgagccatttatacagaaagatgtggaattaagaataatgcctccagttcaggaaaatgacaatgggaatgaaacagaatcagagcagcctaaagaatcaaatgaaaaccaagagaaagaggcagaaaaaactattgattctgaaaaggacctattagatgattcagatgatgatgatattgaaatactagaagaatgtattatttctgccatgccaacaaagtcatcacgtaaagcaaaaaagccagcccagactgcttcaaaattacctccacctgtggcaaggaaaccaagtcagctgcctgtgtacaaacttctaccatcacaaaacaggttgcaaccccaaaagcatgttagttttacaccgggggatgatatgccacgggtgtattgtgttgaagggacacctataaacttttccacagctacatctctaagtgatctaacaatcgaatcccctccaaatgagttagctgctggagaaggagttagaggaggggcacagtcaggtgaatttgaaaaacgagataccattcctacagaaggcagaagtacagatgaggctcaaggaggaaaaacctcatctgtaaccatacctgaattggatgacaataaagcagaggaaggtgatattcttgcagaatgcattaattctgctatgcccaaagggaaaagtcacaagcctttccgtgtgaaaaagataatggaccaggtccagcaagcatctgcgtcttcttctgcacccaacaaaaatcagttagatggtaagaaaaagaaaccaacttcaccagtaaaacctataccacaaaatactgaatataggacacgtgtaagaaaaaatgcagactcaaaaaataatttaaatgctgagagagttttctcagacaacaaagattcaaagaaacagaatttgaaaaataattccaaggtcttcaatgataagctcccaaataatgaagatagagtcagaggaagttttgcttttgattcacctcatcattacacgcctattgaaggaactccttactgtttttcacgaaatgattctttgagttctctagattttgatgatgatgatgttgacctttccagggaaaaggctgaattaagaaaggcaaaagaaaataaggaatcagaggctaaagttaccagccacacagaactaacctccaaccaacaatcagctaataagacacaagctattgcaaagcagccaataaatcgaggtcagcctaaacccatacttcagaaacaatccacttttccccagtcatccaaagacataccagacagaggggcagcaactgatgaaaagttacagaattttgctattgaaaatactccggtttgcttttctcataattcctctctgagttctctcagtgacattgaccaagaaaacaacaataaagaaaatgaacctatcaaagagactgagccccctgactcacagggagaaccaagtaaacctcaagcatcaggctatgctcctaaatcatttcatgttgaagataccccagtttgtttctcaagaaacagttctctcagttctcttagtattgactctgaagatgacctgttgcaggaatgtataagctccgcaatgccaaaaaagaaaaagccttcaagactcaagggtgataatgaaaaacatagtcccagaaatatgggtggcatattaggtgaagatctgacacttgatttgaaagatatacagagaccagattcagaacatggtctatcccctgattcagaaaattttgattggaaagctattcaggaaggtgcaaattccatagtaagtagtttacatcaagctgctgctgctgcatgtttatctagacaagcttcgtctgattcagattccatcctttccctgaaatcaggaatctctctgggatcaccatttcatcttacacctgatcaagaagaaaaaccctttacaagtaataaaggcccacgaattctaaaaccaggggagaaaagtacattggaaactaaaaagatagaatctgaaagtaaaggaatcaaaggaggaaaaaaagtttataaaagtttgattactggaaaagttcgatctaattcagaaatttcaggccaaatgaaacagccccttcaagcaaacatgccttcaatctctcgaggcaggacaatgattcatattccaggagttcgaaatagctcctcaagtacaagtcctgtttctaaaaaaggcccaccccttaagactccagcctccaaaagccctagtgaaggtcaaacagccaccacttctcctagaggagccaagccatctgtgaaatcagaattaagccctgttgccaggcagacatcccaaataggtgggtcaagtaaagcaccttctagatcaggatctagagattcgaccccttcaagacctgcccagcaaccattaagtagacctatacagtctcctggccgaaactcaatttcccctggtagaaatggaataagtcctcctaacaaattatctcaacttccaaggacatcatcccctagtactgcttcaactaagtcctcaggttctggaaaaatgtcatatacatctccaggtagacagatgagccaacagaaccttaccaaacaaacaggtttatccaagaatgccagtagtattccaagaagtgagtctgcctccaaaggactaaatcagatgaataatggtaatggagccaataaaaaggtagaactttctagaatgtcttcaactaaatcaagtggaagtgaatctgatagatcagaaagacctgtattagtacgccagtcaactttcatcaaagaagctccaagcccaaccttaagaagaaaattggaggaatctgcttcatttgaatctctttctccatcatctagaccagcttctcccactaggtcccaggcacaaactccagttttaagtccttcccttcctgatatgtctctatccacacattcgtctgttcaggctggtggatggcgaaaactcccacctaatctcagtcccactatagagtataatgatggaagaccagcaaagcgccatgatattgcacggtctcattctgaaagtccttctagacttccaatcaataggtcaggaacctggaaacgtgagcacagcaaacattcatcatcccttcctcgagtaagcacttggagaagaactggaagttcatcttcaattctttctgcttcatcagaatccagtgaaaaagcaaaaagtgaggatgaaaaacatgtgaactctatttcaggaaccaaacaaagtaaagaaaaccaagtatccgcaaaaggaacatggagaaaaataaaagaaaatgaattttctcccacaaatagtacttctcagaccgtttcctcaggtgctacaaatggtgctgaatcaaagactctaatttatcaaatggcacctgctgtttctaaaacagaggatgtttgggtgagaattgaggactgtcccattaacaatcctagatctggaagatctcccacaggtaatactcccccggtgattgacagtgtttcagaaaaggcaaatccaaacattaaagattcaaaagataatcaggcaaaacaaaatgtgggtaatggcagtgttcccatgcgtaccgtgggtttggaaaatcgcctgaactcctttattcaggtggatgcccctgaccaaaaaggaactgagataaaaccaggacaaaataatcctgtccctgtatcagagactaatgaaagttctatagtggaacgtaccccattcagttctagcagctcaagcaaacacagttcacctagtgggactgttgctgccagagtgactccttttaattacaacccaagccctaggaaaagcagcgcagatagcacttcagctcggccatctcagatcccaactccagtgaataacaacacaaagaagcgagattccaaaactgacagcacagaatccagtggaacccaaagtcctaagcgccattctgggtcttaccttgtgacatctgtttaa

SEQ ID NO: 9 - DNA, 1014 bp, Homo sapiens
  source 1..1014: mol_type = genomic DNA
atggcgagtagcagcggctccaaggctgaattcattgtcggagggaaatataaactggtacggaagatcgggtctggctccttcggggacatctatttggcgatcaacatcaccaacggcgaggaagtggcagtgaagctagaatctcagaaggccaggcatccccagttgctgtacgagagcaagctctataagattcttcaaggtggggttggcatcccccacatacggtggtatggtcaggaaaaagactacaatgtactagtcatggatcttctgggacctagcctcgaagacctcttcaatttctgttcaagaaggttcacaatgaaaactgtacttatgttagctgaccagatgatcagtagaattgaatatgtgcatacaaagaattttatacacagagacattaaaccagataacttcctaatgggtattgggcgtcactgtaataagttattccttattgattttggtttggccaaaaagtacagagacaacaggacaaggcaacacataccatacagagaagataaaaacctcactggcactgcccgatatgctagcatcaatgcacatcttggtattgagcagagtcgccgagatgacatggaatcattaggatatgttttgatgtattttaatagaaccagcctgccatggcaagggctaaaggctgcaacaaagaaacaaaaatatgaaaagattagtgaaaagaagatgtccacgcctgttgaagttttatgtaaggggtttcctgcagaatttgcgatgtacttaaactattgtcgtgggctacgctttgaggaagccccagattacatgtatctgaggcagctattccgcattcttttcaggaccctgaaccatcaatatgactacacatttgattggacaatgttaaagcagaaagcagcacagcaggcagcctcttccagtgggcagggtcagcaggcccaaacccccacaggcaagcaaactgacaaaaccaagagtaacatgaaaggtttctaa

SEQ ID NO: 10 - RNA, 1014 nt, Homo sapiens
  source 1..1014: mol_type = genomic RNA
atggcgagtagcagcggctccaaggctgaattcattgtcggagggaaatataaactggtacggaagatcgggtctggctccttcggggacatctatttggcgatcaacatcaccaacggcgaggaagtggcagtgaagctagaatctcagaaggccaggcatccccagttgctgtacgagagcaagctctataagattcttcaaggtggggttggcatcccccacatacggtggtatggtcaggaaaaagactacaatgtactagtcatggatcttctgggacctagcctcgaagacctcttcaatttctgttcaagaaggttcacaatgaaaactgtacttatgttagctgaccagatgatcagtagaattgaatatgtgcatacaaagaattttatacacagagacattaaaccagataacttcctaatgggtattgggcgtcactgtaataagttattccttattgattttggtttggccaaaaagtacagagacaacaggacaaggcaacacataccatacagagaagataaaaacctcactggcactgcccgatatgctagcatcaatgcacatcttggtattgagcagagtcgccgagatgacatggaatcattaggatatgttttgatgtattttaatagaaccagcctgccatggcaagggctaaaggctgcaacaaagaaacaaaaatatgaaaagattagtgaaaagaagatgtccacgcctgttgaagttttatgtaaggggtttcctgcagaatttgcgatgtacttaaactattgtcgtgggctacgctttgaggaagccccagattacatgtatctgaggcagctattccgcattcttttcaggaccctgaaccatcaatatgactacacatttgattggacaatgttaaagcagaaagcagcacagcaggcagcctcttccagtgggcagggtcagcaggcccaaacccccacaggcaagcaaactgacaaaaccaagagtaacatgaaaggtttctaa

SEQ ID NO: 11 - DNA, 1302 bp, Homo sapiens
  source 1..1302: mol_type = genomic DNA
atgtcagggcggcccagaaccacctcctttgcggagagctgcaagccggtgcagcagccttcagcttttggcagcatgaaagttagcagagacaaggacggcagcaaggtgacaacagtggtggcaactcctgggcagggtccagacaggccacaagaagtcagctatacagacactaaagtgattggaaatggatcatttggtgtggtatatcaagccaaactttgtgattcaggagaactggtcgccatcaagaaagtattgcaggacaagagatttaagaatcgagagctccagatcatgagaaagctagatcactgtaacatagtccgattgcgttatttcttctactccagtggtgagaagaaagatgaggtctatcttaatctggtgctggactatgttccggaaacagtatacagagttgccagacactatagtcgagccaaacagacgctccctgtgatttatgtcaagttgtatatgtatcagctgttccgaagtttagcctatatccattcctttggaatctgccatcgggatattaaaccgcagaacctcttgttggatcctgatactgctgtattaaaactctgtgactttggaagtgcaaagcagctggtccgaggagaacccaatgtttcgtatatctgttctcggtactatagggcaccagagttgatctttggagccactgattatacctctagtatagatgtatggtctgctggctgtgtgttggctgagctgttactaggacaaccaatatttccaggggatagtggtgtggatcagttggtagaaataatcaaggtcctgggaactccaacaagggagcaaatcagagaaatgaacccaaactacacagaatttaaattccctcaaattaaggcacatccttggactaaggattcgtcaggaacaggacatttcacctcaggagtgcgggtcttccgaccccgaactccaccggaggcaattgcactgtgtagccgtctgctggagtatacaccaactgcccgactaacaccactggaagcttgtgcacattcattttttgatgaattacgggacccaaatgtcaaactaccaaatgggcgagacacacctgcactcttcaacttcaccactcaagaactgtcaagtaatccacctctggctaccatccttattcctcctcatgctcggattcaagcagctgcttcaacccccacaaatgccacagcagcgtcagatgctaatactggagaccgtggacagaccaataatgctgcttctgcatcagcttccaactccacctga

SEQ ID NO: 12 - RNA, 1302 nt, Homo sapiens
  source 1..1302: mol_type = genomic RNA
atgtcagggcggcccagaaccacctcctttgcggagagctgcaagccggtgcagcagccttcagcttttggcagcatgaaagttagcagagacaaggacggcagcaaggtgacaacagtggtggcaactcctgggcagggtccagacaggccacaagaagtcagctatacagacactaaagtgattggaaatggatcatttggtgtggtatatcaagccaaactttgtgattcaggagaactggtcgccatcaagaaagtattgcaggacaagagatttaagaatcgagagctccagatcatgagaaagctagatcactgtaacatagtccgattgcgttatttcttctactccagtggtgagaagaaagatgaggtctatcttaatctggtgctggactatgttccggaaacagtatacagagttgccagacactatagtcgagccaaacagacgctccctgtgatttatgtcaagttgtatatgtatcagctgttccgaagtttagcctatatccattcctttggaatctgccatcgggatattaaaccgcagaacctcttgttggatcctgatactgctgtattaaaactctgtgactttggaagtgcaaagcagctggtccgaggagaacccaatgtttcgtatatctgttctcggtactatagggcaccagagttgatctttggagccactgattatacctctagtatagatgtatggtctgctggctgtgtgttggctgagctgttactaggacaaccaatatttccaggggatagtggtgtggatcagttggtagaaataatcaaggtcctgggaactccaacaagggagcaaatcagagaaatgaacccaaactacacagaatttaaattccctcaaattaaggcacatccttggactaaggattcgtcaggaacaggacatttcacctcaggagtgcgggtcttccgaccccgaactccaccggaggcaattgcactgtgtagccgtctgctggagtatacaccaactgcccgactaacaccactggaagcttgtgcacattcattttttgatgaattacgggacccaaatgtcaaactaccaaatgggcgagacacacctgcactcttcaacttcaccactcaagaactgtcaagtaatccacctctggctaccatccttattcctcctcatgctcggattcaagcagctgcttcaacccccacaaatgccacagcagcgtcagatgctaatactggagaccgtggacagaccaataatgctgcttctgcatcagcttccaactccacctga

SEQ ID NO: 13 - protein, 781 aa, Homo sapiens
MATQADLMELDMAMEPDRKAAVSHWQQQSYLDSGIHSGATTTAPSLSGKGNPEEEDVDTSQVLYEWEQGFSQSFTQEQVADIDGQYAMTRAQRVRAAMFPETLDEGMQIPSTQFDAAHPTNVQRLAEPSQMLKHAVVNLINYQDDAELATRAIPELTKLLNDEDQVVVNKAAVMVHQLSKKEASRHAIMRSPQMVSAIVRTMQNTNDVETARCTAGTLHNLSHHREGLLAIFKSGGIPALVKMLGSPVDSVLFYAITTLHNLLLHQEGAKMAVRLAGGLQKMVALLNKTNVKFLAITTDCLQILAYGNQESKLIILASGGPQALVNIMRTYTYEKLLWTTSRVLKVLSVCSSNKPAIVEAGGMQALGLHLTDPSQRLVQNCLWTLRNLSDAATKQEGMEGLLGTLVQLLGSDDINVVTCAAGILSNLTCNNYKNKMMVCQVGGIEALVRTVLRAGDREDITEPAICALRHLTSRHQEAEMAQNAVRLHYGLPVVVKLLHPPSHWPLIKATVGLIRNLALCPANHAPLREQGAIPRLVQLLVRAHQDTQRRTSMGGTQQQFVEGVRMEEIVEGCTGALHILARDVHNRIVIRGLNTIPLFVQLLYSPIENIQRVAAGVLCELAQDKEAAEAIEAEGATAPLTELLHSRNEGVATYAAAVLFRMSEDKPQDYKKRLSVELTSSLFRTEPMAWNETADLGLDIGAQGEPLGYRQDDPSYRSFHSGGYGQDALGMDPMMEHEMGGHHPGADYPVDGLPDLGHAQDLMDGLPPGDSNQLAWFDTDL

SEQ ID NO: 14 - protein, 862 aa, Homo sapiens
MNIQEQGFPLDLGASFTEDAPRPPVPGEEGELVSTDPRPASYSFCSGKGVGIKGETSTATPRRSDLDLGYEPEGSASPTPPYLKWAESLHSLLDDQDGISLFRTFLKQEGCADLLDFWFACTGFRKLEPCDSNEEKRLKLARAIYRKYILDNNGIVSRQTKPATKSFIKGCIMKQLIDPAMFDQAQTEIQATMEENTYPSFLKSDIYLEYTRTGSESPKVCSDQSSGSGTGKGISGYLPTLNEDEEWKCDQDMDEDDGRDAAPPGRLPQKLLLETAAPRVSSSRRYSEGREFRYGSWREPVNPYYVNAGYALAPATSANDSEQQSLSSDADTLSLTDSSVDGIPPYRIRKQHRREMQESVQVNGRVPLPHIPRTYRVPKEVRVEPQKFAEELIHRLEAVQRTREAEEKLEERLKRVRMEEEGEDGDPSSGPPGPCHKLPPAPAWHHFPPRCVDMGCAGLRDAHEENPESILDEHVQRVLRTPGRQSPGPGHRSPDSGHVAKMPVALGGAASGHGKHVPKSGAKLDAAGLHHHRHVHHHVHHSTARPKEQVEAEATRRAQSSFAWGLEPHSHGARSRGYSESVGAAPNASDGLAHSGKVGVACKRNAKKAESGKSASTEVPGASEDAEKNQKIMQWIIEGEKEISRHRRTGHGSSGTRKPQPHENSRPLSLEHPWAGPQLRTSVQPSHLFIQDPTMPPHPAPNPLTQLEEARRRLEEEEKRASRAPSKQRYVQEVMRRGRACVRPACAPVLHVVPAVSDMELSETETRSQRKVGGGSAQPCDSIVVAYYFCGEPIPYRTLVRGRAVTLGQFKELLTKKGSYRYYFKKVSDEFDCGVVFEEVREDEAVLPVFEEKIIGKVEKVD

SEQ ID NO: 15 - protein, 843 aa, Homo sapiens
MSSAMLVTCLPDPSSSFREDAPRPPVPGEEGETPPCQPGVGKGQVTKPMPVSSNTRRNEDGLGEPEGRASPDSPLTRWTKSLHSLLGDQDGAYLFRTFLEREKCVDTLDFWFACNGFRQMNLKDTKTLRVAKAIYKRYIENNSIVSKQLKPATKTYIRDGIKKQQIDSIMFDQAQTEIQSVMEENAYQMFLTSDIYLEYVRSGGENTAYMSNGGLGSLKVVCGYLPTLNEEEEWTCADFKCKLSPTVVGLSSKTLRATASVRSTETVDSGYRSFKRSDPVNPYHIGSGYVFAPATSANDSEISSDALTDDSMSMTDSSVDGIPPYRVGSKKQLQREMHRSVKANGQVSLPHFPRTHRLPKEMTPVEPATFAAELISRLEKLKLELESRHSLEERLQQIREDEEREGSELTLNSREGAPTQHPLSLLPSGSYEEDPQTILDDHLSRVLKTPGCQSPGVGRYSPRSRSPDHHHHHHSQYHSLLPPGGKLPPAAASPGACPLLGGKGFVTKQTTKHVHHHYIHHHAVPKTKEEIEAEATQRVHCFCPGGSEYYCYSKCKSHSKAPETMPSEQFGGSRGSTLPKRNGKGTEPGLALPAREGGAPGGAGALQLPREEGDRSQDVWQWMLESERQSKPKPHSAQSTKKAYPLESARSSPGERASRHHLWGGNSGHPRTTPRAHLFTQDPAMPPLTPPNTLAQLEEACRRLAEVSKPPKQRCCVASQQRDRNHSATVQTGATPFSNPSLAPEDHKEPKKLAGVHALQASELVVTYFFCGEEIPYRRMLKAQSLTLGHFKEQLSKKGNYRYYFKKASDEFACGAVFEEIWEDETVLPMYEGRILGKVERID

SEQ ID NO: 16 - protein, 2843 aa, Homo sapiens
MAAASYDQLLKQVEALKMENSNLRQELEDNSNHLTKLETEASNMKEVLKQLQGSIEDEAMASSGQIDLLERLKELNLDSSNFPGVKLRSKMSLRSYGSREGSVSSRSGECSPVPMGSFPRRGFVNGSRESTGYLEELEKERSLLLADLDKEEKEKDWYYAQLQNLTKRIDSLPLTENFSLQTDMTRRQLEYEARQIRVAMEEQLGTCQDMEKRAQRRIARIQQIEKDILRIRQLLQSQATEAERSSQNKHETGSHDAERQNEGQGVGEINMATSGNGQGSTTRMDHETASVLSSSSTHSAPRRLTSHLGTKVEMVYSLLSMLGTHDKDDMSRTLLAMSSSQDSCISMRQSGCLPLLIQLLHGNDKDSVLLGNSRGSKEARARASAALHNIIHSQPDDKRGRREIRVLHLLEQIRAYCETCWEWQEAHEPGMDQDKNPMPAPVEHQICPAVCVLMKLSFDEEHRHAMNELGGLQAIAELLQVDCEMYGLTNDHYSITLRRYAGMALTNLTFGDVANKATLCSMKGCMRALVAQLKSESEDLQQVIASVLRNLSWRADVNSKKTLREVGSVKALMECALEVKKESTLKSVLSALWNLSAHCTENKADICAVDGALAFLVGTLTYRSQTNTLAIIESGGGILRNVSSLIATNEDHRQILRENNCLQTLLQHLKSHSLTIVSNACGTLWNLSARNPKDQEALWDMGAVSMLKNLIHSKHKMIAMGSAAALRNLMANRPAKYKDANIMSPGSSLPSLHVRKQKALEAELDAQHLSETFDNIDNLSPKASHRSKQRHKQSLYGDYVFDTNRHDDNRSDNFNTGNMTVLSPYLNTTVLPSSSSSRGSLDSSRSEKDRSLERERGIGLGNYHPATENPGTSSKRGLQISTTAAQIAKVMEEVSAIHTSQEDRSSGSTTELHCVTDERNALRRSSAAHTHSNTYNFTKSENSNRTCSMPYAKLEYKRSSNDSLNSVSSSDGYGKRGQMKPSIESYSEDDESKFCSYGQYPADLAHKIHSANHMDDNDGELDTPINYSLKYSDEQLNSGRQSPSQNERWARPKHIIEDEIKQSEQRQSRNQSTTYPVYTESTDDKHLKFQPHFGQQECVSPYRSRGANGSETNRVGSNHGINQNVSQSLCQEDDYEDDKPTNYSERYSEEEQHEEEERPTNYSIKYNEEKRHVDQPIDYSLKYATDIPSSQKQSFSFSKSSSGQSSKTEHMSSSSENTSTPSSNAKRQNQLHPSSAQSRSGQPQKAATCKVSSINQETIQTYCVEDTPICFSRCSSLSSLSSAEDEIGCNQTTQEADSANTLQIAEIKEKIGTRSAEDPVSEVPAVSQHPRTKSSRLQGSSLSSESARHKAVEFSSGAKSPSKSGAQTPKSPPEHYVQETPLMFSRCTSVSSLDSFESRSIASSVQSEPCSGMVSGIISPSDLPDSPGQTMPPSRSKTPPPPPQTAQTKREVPKNKAPTAEKRESGPKQAAVNAAVQRVQVLPDADTLLHFATESTPDGFSCSSSLSALSLDEPFIQKDVELRIMPPVQENDNGNETESEQPKESNENQEKEAEKTIDSEKDLLDDSDDDDIEILEECIISAMPTKSSRKAKKPAQTASKLPPPVARKPSQLPVYKLLPSQNRLQPQKHVSFTPGDDMPRVYCVEGTPINFSTATSLSDLTIESPPNELAAGEGVRGGAQSGEFEKRDTIPTEGRSTDEAQGGKTSSVTIPELDDNKAEEGDILAECINSAMPKGKSHKPFRVKKIMDQVQQASASSSAPNKNQLDGKKKKPTSPVKPIPQNTEYRTRVRKNADSKNNLNAERVFSDNKDSKKQNLKNNSKVFNDKLPNNEDRVRGSFAFDSPHHYTPIEGTPYCFSRNDSLSSLDFDDDDVDLSREKAELRKAKENKESEAKVTSHTELTSNQQSANKTQAIAKQPINRGQPKPILQKQSTFPQSSKDIPDRGAATDEKLQNFAIENTPVCFSHNSSLSSLSDIDQENNNKENEPIKETEPPDSQGEPSKPQASGYAPKSFHVEDTPVCFSRNSSLSSLSIDSEDDLLQECISSAMPKKKKPSRLKGDNEKHSPRNMGGILGEDLTLDLKDIQRPDSEHGLSPDSENFDWKAIQEGANSIVSSLHQAAAAACLSRQASSDSDSILSLKSGISLGSPFHLTPDQEEKPFTSNKGPRILKPGEKSTLETKKIESESKGIKGGKKVYKSLITGKVRSNSEISGQMKQPLQANMPSISRGRTMIHIPGVRNSSSSTSPVSKKGPPLKTPASKSPSEGQTATTSPRGAKPSVKSELSPVARQTSQIGGSSKAPSRSGSRDSTPSRPAQQPLSRPIQSPGRNSISPGRNGISPPNKLSQLPRTSSPSTASTKSSGSGKMSYTSPGRQMSQQNLTKQTGLSKNASSIPRSESASKGLNQMNNGNGANKKVELSRMSSTKSSGSESDRSERPVLVRQSTFIKEAPSPTLRRKLEESASFESLSPSSRPASPTRSQAQTPVLSPSLPDMSLSTHSSVQAGGWRKLPPNLSPTIEYNDGRPAKRHDIARSHSESPSRLPINRSGTWKREHSKHSSSLPRVSTWRRTGSSSSILSASSESSEKAKSEDEKHVNSISGTKQSKENQVSAKGTWRKIKENEFSPTNSTSQTVSSGATNGAESKTLIYQMAPAVSKTEDVWVRIEDCPINNPRSGRSPTGNTPPVIDSVSEKANPNIKDSKDNQAKQNVGNGSVPMRTVGLENRLNSFIQVDAPDQKGTEIKPGQNNPVPVSETNESSIVERTPFSSSSSSKHSSPSGTVAARVTPFNYNPSPRKSSADSTSARPSQIPTPVNNNTKKRDSKTDSTESSGTQSPKRHSGSYLVTSV

SEQ ID NO: 17 - protein, 337 aa, Homo sapiens
MASSSGSKAEFIVGGKYKLVRKIGSGSFGDIYLAINITNGEEVAVKLESQKARHPQLLYESKLYKILQGGVGIPHIRWYGQEKDYNVLVMDLLGPSLEDLFNFCSRRFTMKTVLMLADQMISRIEYVHTKNFIHRDIKPDNFLMGIGRHCNKLFLIDFGLAKKYRDNRTRQHIPYREDKNLTGTARYASINAHLGIEQSRRDDMESLGYVLMYFNRTSLPWQGLKAATKKQKYEKISEKKMSTPVEVLCKGFPAEFAMYLNYCRGLRFEEAPDYMYLRQLFRILFRTLNHQYDYTFDWTMLKQKAAQQAASSSGQGQQAQTPTGKQTDKTKSNMKGF

SEQ ID NO: 18 - protein, 433 aa, Homo sapiens
MSGRPRTTSFAESCKPVQQPSAFGSMKVSRDKDGSKVTTVVATPGQGPDRPQEVSYTDTKVIGNGSFGVVYQAKLCDSGELVAIKKVLQDKRFKNRELQIMRKLDHCNIVRLRYFFYSSGEKKDEVYLNLVLDYVPETVYRVARHYSRAKQTLPVIYVKLYMYQLFRSLAYIHSFGICHRDIKPQNLLLDPDTAVLKLCDFGSAKQLVRGEPNVSYICSRYYRAPELIFGATDYTSSIDVWSAGCVLAELLLGQPIFPGDSGVDQLVEIIKVLGTPTREQIREMNPNYTEFKFPQIKAHPWTKDSSGTGHFTSGVRVFRPRTPPEAIALCSRLLEYTPTARLTPLEACAHSFFDELRDPNVKLPNGRDTPALFNFTTQELSSNPPLATILIPPHARIQAAASTPTNATAASDANTGDRGQTNNAASASASNST

SEQ ID NO: 19 - DNA, 99 bp, synthetic construct
  source 1..99: mol_type = other DNA
cagtcttacctggattctggcatccacagcggagccaccaccaccgccccttatctgagcggcaagggaaacccagaggaggaggatgtggatacatcc

SEQ ID NO: 20 - RNA, 99 nt, synthetic construct
  source 1..99: mol_type = other RNA
cagtcttacctggattctggcatccacagcggagccaccaccaccgccccttatctgagcggcaagggaaacccagaggaggaggatgtggatacatcc

SEQ ID NO: 21 - DNA, 99 bp, synthetic construct
  source 1..99: mol_type = other DNA
cctgacaggaaggccgccgtgtcccactggcagcagcagagctatctgggcagcggcatccactctggcgccacaaccaccgcaccatctctgtctggg

SEQ ID NO: 22 - RNA, 99 nt, synthetic construct
  source 1..99: mol_type = other RNA
cctgacaggaaggccgccgtgtcccactggcagcagcagagctatctgggcagcggcatccactctggcgccacaaccaccgcaccatctctgtctggg

SEQ ID NO: 23 - DNA, 78 bp, synthetic construct
  source 1..78: mol_type = other DNA
gagcagcagtctctgtctagcgacgccgataccctgtctctgaccgactctagcgtggacccacctatccaggaccca

SEQ ID NO: 24 - RNA, 78 nt, synthetic construct
  source 1..78: mol_type = other RNA
gagcagcagtctctgtctagcgacgccgataccctgtctctgaccgactctagcgtggacccacctatccaggaccca

SEQ ID NO: 25 - DNA, 99 bp, synthetic construct
  source 1..99: mol_type = other DNA
cctgatcggaaagccgcagtgagccactggcagcagcagtcctacctgggcagcggaatccactctggagccacaacaaccgcaccatctctgtctggc

SEQ ID NO: 26 - RNA, 99 nt, synthetic construct
  source 1..99: mol_type = other RNA
cctgatcggaaagccgcagtgagccactggcagcagcagtcctacctgggcagcggaatccactctggagccacaacaaccgcaccatctctgtctggc

SEQ ID NO: 27 - DNA, 99 bp, synthetic construct
  source 1..99: mol_type = other DNA
cagtcctatctggacagcggcatccactccggcgcaacaaccacagcacctccactgtctggaaagggaaatcctgaggaggaggatgtggatacatcc

SEQ ID NO: 28 - RNA, 99 nt, synthetic construct
  source 1..99: mol_type = other RNA
cagtcctatctggacagcggcatccactccggcgcaacaaccacagcacctccactgtctggaaagggaaatcctgaggaggaggatgtggatacatcc

SEQ ID NO: 29 - DNA, 99 bp, synthetic construct
  source 1..99: mol_type = other DNA
tcttatctggattccggaattcactccggcgccaccaccacagcacccttcctgtctggaaaaggaaatcctgaggaggaggacgtggacacaagccag

SEQ ID NO: 30 - RNA, 99 nt, synthetic construct
  source 1..99: mol_type = other RNA
tcttatctggattccggaattcactccggcgccaccaccacagcacccttcctgtctggaaaaggaaatcctgaggaggaggacgtggacacaagccag

SEQ ID NO: 31 - protein, 33 aa, synthetic construct
QSYLDSGIHSGATTTAPYLSGKGNPEEEDVDTS

SEQ ID NO: 32 - protein, 33 aa, synthetic construct
PDRKAAVSHWQQQSYLGSGIHSGATTTAPSLSG

SEQ ID NO: 33 - protein, 26 aa, synthetic construct
EQQSLSSDADTLSLTDSSVDPPIQDP

SEQ ID NO: 34 - protein, 33 aa, synthetic construct; identical to SEQ ID NO: 32

SEQ ID NO: 35 - protein, 33 aa, synthetic construct
QSYLDSGIHSGATTTAPPLSGKGNPEEEDVDTS

SEQ ID NO: 36 - protein, 33 aa, synthetic construct
SYLDSGIHSGATTTAPFLSGKGNPEEEDVDTSQ

SEQ ID NO: 37 - DNA, 4740 bp, synthetic construct
  source 1..4740: mol_type = other DNA
atggattggacatggattctctttctcgttgcagcagccacacgcgttcacagtcagtcttacctggattctggcatccacagcggagccaccaccaccgccccttatctgagcggcaagggaaacccagaggaggaggatgtggatacatccagaggccgcaagcggagatctcacctggtgtccagaaagtccatctctgccgccctggagcacaaggtgagcgccctgctgccccccgccgagcagaccggaacatggaagctgaaaaccagaggcaggaagagaagatccgacctgcggtcccgcatcgaggtgctgaagaggaaggtgatcgagaagctgcagcacatccagctgctgcagaagaatgtgcgggcacagctggtggaccggggaaggaagcggcgcagcagcaaggacctgctgcggcagctgcctgcctccaactttaactctctgcgcttcctgatcgtgcacctgaaaagagtggtggatcacgccgaggagaatcggggcagaaagcgccgctccaccctgctgctggatggcaaccctttcagagtgccccaggccgcaatcctgatgaagggcaccgccgcaatcctggagtacctgagggacagaatccctaggggcaggaagcggcggtcctccatcctggagagcagctccgcctttcccgataatgccgcccgcagggaggtggagagccccccagcagtctgtccatctgacggctgcacatggaagagaggacgcaagcggagatccgggcagccatacttcgaggcattcaagaagaagatgcccatcgcattcgtggcaaagggcatcaataagctgctgaacaagctgtttctgatcaacgagaggggccggaagcgcaggagcatggaggatctgcaggacatgtttatcgtgcacacaatcgaggagatcgagggcctgattgcagcccacgatcagttcaagagcaccctgccagacgccagaggccggaagaggcgcagcatgggcggccagaccgccctgaattgcggcgaggagctgtttaagagaggcgtgctgaaggagtacggcgtgaaggtgctgggcacctctgtggagagccgcggcaggaagcggcgctccgccaagggcgtgaacggctctagccaggcccctaccaccggaaagtactgtcgcctgtgcgacatccagtttaataatctgagcaactttatcacccacagaggacggaagcggcggagccacatgaagctgaaagtgcagggaatggagtgtctggatggctgttatgtccacagcagcatcgtgccaacattccatagaagatgcacatggctgctgcgcggcagaaaaaggagatctcaggcaagcagaggcgaggtgccaagcggcgcctacggctacagctatataccttccggagcctatgtgtaccccccacctgtggccaacggcatgtataggggcaggaagagaaggagctattccagcctgagatctatcgtggtggccaattacgaggagtccatcaagatgccaatcagcgagccagcccctggcaagaagaagtcccagatccagcgcggcaggaagaggcgctctgacctgcaggtgagcgccaagatcggcttcctgaagcaccacgagaataagacatacctggccgtgggctctatgaaaaccctgatgctgaacgtgagcaggggcagaaagcgcaggtccgagctgatcctgcctagcggcgccagggtgggccacaggagcctgaggagatactataagcagaggttcggcctgtctagagccgtggccgtggccaagcggggacgcaagaggaggtctatgctgggcgagcagggattccagtccctgatggaaaccgtggatacaggcatcgtgacacagctgcaggagtttctgcagggattcaggggccgcaagaggaggtcccgccagatctggtgggagagactgcagggctgccagcacatcgtggaggattggcagaagatcctgatggtgcgctccctggtggtgagcccccacgagagaggacggaagcgcaggtccagcaccaccggatctaactactacgtgagaatcctgtctacaatcgatggcgagctgctgaagccaaatgccagcgtggccctgcacaagcactccaacaggggccgcaagcgcaggagcgccaacgagctgtgcgaggtgaacagaaagggctgcccaagcgccgatccatgtctgccttacttctgcgtgcagggctgtaagctgggcgaggcctcccggggacgcaagcggaggtcttttgaggattgcacatctgagaacaccctgatcaagtatatgaccgatcgcatcctgctgagggagtctctgcgcgaggcagacctggaccactatagccggggaaggaaaaggagaagctggatcagaccactggcatccggcagctaccctagccgcaaggcaggagccacactggtggtgtacaaggatctgctggtgctgttcgggggctggacacgcggacgcaagagaaggagctcctccgactactccacatccgagatgctggtgaacgtgggaaacctgcccctggacgagttttatcccgccgtgagcatggtggccctgatgcgcatccggggccgcaagagaagatcctgcgagagccccacagcacactgtaatgtgctgaattgggagcaggtgcacagactggacggcatcctgtctgagacaatcccaatccacggaaggggaagggggaggaagagaaggtccaataagcagaataagaccccactggataagtctacatccggggtgtctgagatcctgctgaaaacccagatgaaaatgagcctgaagtgcctggccgcccgcggacggaagagaaggtcttacgtgtgcggcggcatgttcttctttgtgagcttcgaggggaagatcgtgatgcacaagatcatctatatggtgctgttcctgttttgtgtggcactgagaggcagaaagaggaggtccctgcctaccccagggtcttggtgggagcagctgacccaggcatctcgggcctacgcaagcggaggcaccgagggctttcctctgagccggtgggcccccaggggcagaaagagaaggtcttattccaccctgtccctgtttgacaagtacaagggcaagtccgtggataccatccggagctccgtgatcccccgccacggcctgcagtccctgggcaaaagaggcagaaagagaagaagcatgaacatctctctggtgtggtgcctgctggtgctgtccttcgccatcaggaggtggtggcgcggcaggaagcgccggagcagggtggagctgcagcaggaggtggagaagctggcacacgagaatagctccatgcggctggagctggatgccctgaggtccaaatatgaggcactgcagagggggagaaagagaaggtccagccctcagagagagccaagaggcggccagctgagaaccccaagaatgtggccttcttgttccagaagcctggagtctctgagagtgggagccaagccccggggaaggaagagacgctccttcacactgtggaacgagagcacccactatcagatcctgctgacctcttttccccacgtggagaatcactcttgttttgagcacatgcaccacatcccaagaggcagaaagcggcggtctgattttgtggtggaggccatcggaaccgaggtgggcaccctgggattctccatcgaaagaccttcccaggccaagatcgagtgcgacgacaagggcgatcggggcaggaagcgcagatccaacgtgatcgacagcatgctgttcgtgttcggatatagagcccagaagaataagatccgcggaaggaagaggcggtctatgatgggcagcgcaagggtggcagagctgctgctgctgcacggagcagagcccaactgtaccgaccctgcaacactgaccagaccagtgcacgacgcaagaggaaggaagagaagaagccccccaggcctggacaagagactgctgccagagacactgggcccctgctactccaattctcagcctgtgtggctgtgtctgacaccacggcagccactgagaggcaggaaaagaagatcttccttcctgctgagccctaataatggcaacctggaggccacatctaatatcgagtacgcacacgtgccccacctgtctcctgccgtgatccctacacggaggggccggaagaggaggtctgaggaggatgaggaggacacctattatacaaaggacctgcccatccacacctgcagctactgcggaatccacgacccagcctgcgtggtgtattgtaaccgcggcaggaagagacggagcctgatctctaggagggatgccgaggtggtgctgacaagcagggagctgggcagcctggccctgaaccagtctacaggcctgcctaccctgaccctgccaagaggccggaagcggaggtctgacttctcttctaacgactcctccacatgcaccatgggcctggtgctgacactgtggtctgataccctgatccacctggacggcgatggaggctttagcagaggcagaaaaaggaggtcctccagcgcagccccatccgtgatggacctgagctcccgggcatctgccgcactgcacccaggcatcccctcccctaattgtatgcagtctcccatccgc

SEQ ID NO: 38 - DNA, 4722 bp, synthetic construct
  source 1..4722: mol_type = other DNA
atggattggacctggattctctttcttgttgctgctgccactcgcgttcacagtcctgacaggaaggccgccgtgtcccactggcagcagcagagctatctgggcagcggcatccactctggcgccacaaccaccgcaccatctctgtctgggagaggcaggaaacggagaagcggccagtctcctacactgctgacatctgcctgtttcttcccatctgcagcctttacagccgagtttctgtttagcctgggctccctgtgtcagcacctgcggggccggaagagaagatctcgcctggccagcgcacctccacagaaacagaagcagaagctgggcgagaggctgttccccctgatccaggcaatgcacccaacactggccggcaaaatcaggggcagaaagaggaggtcttgcggccacctgtatgcctacgattggatctctatcccagtggtgtacacccaggtggtgaccgtggccgtgtatagcttttttctgacctgcctggtgaggggcaggaagagacgcagcgactgtatgtccgaggatggatataagctggataggatcaagggagagttcgagttccacaatgtgacctttcactatcccagccgccccgaggtgaagagaggaaggaagcggaggtctatggccaacgattcccctgccaagtccctggtggacatcgacctgccttccctgagggacccagccggcatcttcgagctggtggaggtggtgggaaataggggccgcaagagacgcagctggctgggcatccggatctctgagtctccagagccagggcagcggacattcacaacctttaagttttgccagctgccattctacaagtattccgagtatagaggcagaaagcggaggagcccccccctggcaagaccaatcctgccaagggagcgcggcgcaatggatagaatcgtggaatacctggtgggagacggccctcagaacagatacgcactgagaggccggaaaaggagaagcacctgcgtgtccctggatgacgggctgtacgagtgtagctgtgcccctgcctattccggaaaggattgtcagaagaaggatggaccttgcgtgatcaacaggggcaggaagcgcagaagcctgcacgtgggcctgtgttccggcccctgtgagatggccgagcagcggtgctgcgtggactacgccaagaggggcaccgccgggtgtaagaagtgtaagaggggccggaagcggcgctccagcttcgagagctacctgaaaacaatgccaccatactttctgggcccactgaagaaggcagtgcgcatgatgggagccccaaacctgatcgcagacagcaggggcagaaagcgccgcagccctaagttccactcctacgtgtctctgccattcggctgcacaagggccgtggtggagtacaggctgctgcaggccgcatacctggccaagcccggcgacagaggccggaagcgccgctccggagagtacgtggatacaggcaagctgatcgataagatcaatctgcctaatttcctgaaggtgtatctgaaccacaagccaccttttggcaacaccatgcggggcaggaaaagaagatctatgtgcaccaacgccagaagggtgagaaagagatggctgcctaagatcaacagcatgctgcccgagggagtggagatgtaccgcaccgtgatgggatctagagggagaaagagaagaagcccagacggcccactgctgccatctcccggcctgctgagactgccaggcgggcctcggcacgccgtgtctggagtgcctgaggtgtacccccctggccctagaggcaggaagcggcggagcgtgggcacacaggccaccaagtataatagcgaggtggtggacaagatgatcgaggagtttctgtccagctttgaggagaagatcgagaatctgacagagaggggacggaagcgccggagcacaaacctgcctatctttaagctgaaggagagctgcgtgcgcaggagatactctgatttcaagtggctgaagaatgagctggagagagactctaagatcagaggcaggaaaaggcgctccctgatcagatggtgtctggcactgcgcccaagcgacaggccaaccttcgaggagatccagaagcacccatggatgcaggacgtgctgctgccccaggagcggggccggaagcgcagatccaagtttgaatgcggctggtgtagcggcgagaggaggtgtacactgcaccagcactgcacaaacccaagctccccatggctggattggtccagccacaacaggggacggaagagacggagccacgaccacaccggcttcctgaccgagtacgtggccacaaggtggtacagcgcacccgagatcatgctgaattccaaaggctacacaaagtctatcgataggggaaggaagcgccgcagctcctccgaggcaaccgaggtgaatccagagtctctggcaaaggagtgcctggagaaatctctgctgctgctgaagttcctgcccaccggcatcagctccaggggaaggaagaggagaagcgtgaagaaggatagatccgcctctcacctggaccacaaactggagatccgccagtggaggaggtcctttctggtgtccctgtttttctgcatccccgtgcgcggacgcaagcgcaggagcctgtttacattcgccgtgggagtgaatatctgcctgggctttaccgcctacaggatcaagagagcagagggatgggaggagggccctcccaccgtgctgcggggcaggaagaggagatccatgagccaggcccctggcgcacagctgtctcccccaaccgtgtaccacgagagacagaggctggagctgtgtgcaaggggcagaaagcggcgctccatgtcttctaacagctttccctacatcgagcagtccggaggaggcgaggccaccgagctgggccaggaggccaccaggggaaggaaaagaaggtcccagccttgtcgcaagatgctgcctgatcggtccagagccgccagatacagggccaatcagaagctggtggagtatatcgtgaaggcaaagggagccgagaggggccgcaagcggagaagccctgtctttcacggcatgctggagagagcaccagccgagccttgctatcgggccccaatggagaagctgttttatctgccacacgtgtgctcttatacccggggccgcaagcggcgctccacctactcttgcgtggccgagaatatcctgggcacaggacagagaggctccggcgccgagctggatgtgcagtatccccccaagaaggtgaccaccgtgagaggcaggaagcgccgctccagccccagcgcaacagaggattccctgcagcctgccacagacctgctgaacaggtctgagctgccccagagccagaaggcaatgcagaccaaggatgcccgcggcagaaagagaaggtctgacctgagaaaggtgcccggcacagaccccgccttcgaggtgtttcccctgcacgatcaccagtgtgtgctgcggggccgcaagcgccggagcgacgcactggatttcaagaaggataagggcgccttctatctggtgttcatctggaccatgaccagaggcaggaagagaagatccgtggataactacaagcagatcaaggccgacctggatcagctgaggctgcctgtcgagaagtccgagctgtgggtggagaagagctctaactatgagaataggggcagaaagagaagatccaatgcccccgagaatggatatatgaagtgctccagcgagggcgacaactacggcgccacctgcgagttctcttgcatcggaggctatgagctgcagggccggggccgcaagagaagatccgagagcggaagcagcggcaagagctcctcttccctggtgcggggagtccacctgagcagctccggacccgccctgctggccggcctggtgtccctggacaggggccggaagaggagatcttggacagagaagcgggcctcttatgagctggagttcgccaagtccacaacaaagatcgccgaggccggcaaggtgtccatccagcaacagtctcacatgagaggcagaaagcgcaggagcaagatcctggcaaggaagaagtccaaacggtctgcactggagaactccgaggagcactccacaaagtactctaattccaacaactctgccggctccgggagaggcagaaaaagaaggtccgcagcagcagccgccgcactggccgcagccctgctgctgctgaggcgcgaggacccaggactgggggccggccccagcatggcagagacagaggccctgcgcggcagaaagaggagatctctgccttcttttcctcctttcggctctatgaactctaacgccgcaggcagcgtgagcacccaggccaataccgtgcagagcggacagctgggaggacagagaggcagaaagagacggtccagcggagtggtgcaccggctggaggtggacgaggacttcgaggagcacaatgccgcaaaggtgcatctgatggtgcacaatctggtgccaccattcctgaggggcaggaagcggaggagcctgcagatcaagcagcaggagctgtataagaacttcctgagattccaggtggaggagaagaagcagcgggaggaggccgagcgcgagcgcctgagaatc

SEQ ID NO: 39 - DNA, 3465 bp, synthetic construct
  source 1..3465: mol_type = other DNA
atggattggacatggattctttttctcgttgcagcagctacacgcgttcatagcgagcagcagtctctgtctagcgacgccgataccctgtctctgaccgactctagcgtggacccacctatccaggacccaaggggcagaaaaaggagatctgaccagaacaccgaggattacctgtttcaggtgatcctggagaagcagatcagaatcccccacagcctgagcgtgaaggccgcctccgtgctgaagtctagaggccggaagagaaggagcctgagagaggatggccgggagcaggagatccgcgacggcggctccctgcggatgatccacttcggcctggtgagaggccgcaagagacggagcctgctgcagctgtggccagtgcagcggctgacagtgaagaagtccaccgacaatctgttcagagagcagaaatcctccctgaactacctgcgccagaagcggggcagaaagcgccggtcccagtggctgagggccgtggcagtggagagcatccaccgcttctgtatgcagccacagctgctgagatccttctgtcagagctatgatatgaagcagcacagaggaaggaagcggaggagcctggaggccatcgtgcagctgtggcgcatcccatccttcgtgaccgagctgtatgtgaattatgattgtgattattattgctccaatctgttcgaggagagggggagaaagcgcagatccacccctcccggccagtccccaggctccgcccccatcatcatcaacatccacagagcccagctgacaaacccagaggtgaagtacaattataccgaggacaggggacggaagagacgctctccttatgccacagtggcaccctccaccctggcccaccctcaggcccaggtgctggccaggcagcaggccctgcagcacgcccagaccctggcccacgccagaggcagaaaacggagaagccagaggggcctgctgcagcagatcggcgacgcactgtccagccagaggggacgggtccccccagccgcaccccctgcacagcccagggtgccagtgaccagaggcagaaagcgcaggtccatgtttaggtctaagagatccggcctggtgaggaggctgtggcggtccggcgtggtgcccgatcgcgaggagggaggaagcggcggcggcgggggaggccgcggcagaaagaggcggtccctggagatcaaggtgagcccacccgagggcgcagagacacgcaaggtgagagagatcggccctctgtgcggacgcagaaggccacgggtgagaaagtccaggggcaggaagagaaggtctacccctaactttatcatcccagcccagagagccgagcctatgaggatcgtgcgccagcctatgccaccccccggggacctggagcctcctttccagcctagaggcagaaagcggcgctctggcacaggcgccctgggcctgctgctgctgttcctgctgggctatggactgaggagaaggcctcagaaggccgaggagcctgcacctcaggcaggcggccggggacggaagagaagatctgacagccacgtgtccgaggtgctgcaggagaactatcggctgaaaaacaagctggagggactgatcagcgagaagaatgagctgaagatgaagagcgagaggggacggaagcggagatctgtgtctgcataccaagtgcagagcccaagctggatgcagcctcagccatgtatcctgcagcaccctggcgccgtgctgaccccttctatggagcacaccaggggccggaaacggaggagcggcaggttcagcatcgtgaagaagtgcatccacaaggcaaccaggaagtacgtggccgtgaagttcgtgtccaagaagatgaaaaagaaggagcaggcccgcggacgcaagcgccggtctggcatcctgcgcgacccaggctctgagatcgaggacagacagtatagaatcgatctgcagtccatcaatatcggaaccgcccagtggcaccagctgaagagaggaaggaagagaaggtccgacggaacacaggtgctgaagatctacgtgaagcagacagagtcccactacatcctggtgtcttggaaggtgaattctaacgtgatgacatctaatctgagaggccgcaagcgccggagccccccaggatacatccccgatgagctgcaccaggtggcccggaacggcctgtttacatctatcaactctgagggcgagttcatccccgagagcatggataggggcagaaagagacggagcccaaagtttcggggagtgaaaatgatccctccaggcattcacttcctgcactatagcagcatggacaaagccaatcctaaggaggtgggcccacgcatgagaggcagaaagagaagatccaatgtgagcagcgccgagggatggcacgtgaacgtgaccctgtctatcaggccctccacaggcaccggcgtgatgctggcactggtgagcggaaataacaggggccgcaagaggagatcttgtcagaggtcttttaggacctttcaggccctgaagaagcacctggaggcctcccacctggagctgagcgaggccgatatccagcagctgtacggcggccgcggcaggaagcgcagatccgactccgtggactttcgcctgagggtgagcgagcccaaggcagtgttcgccaaggagcagctggcatgcagggaggtgcaggcagaggtgggcgcctctagaggcaggaagaggcgcagcgatatcaaggatggaagatttccttacggatctacccaggattacctgaagccaatcatcctgatcaagctggtgcagctgggcatggcaaaggatgacaggggcagaaagaggcgctccgagaactaccggaacctggcctttctggggatcgcactgtctaagccagacctgatcacattcctggagcagggcaaggagccctggaatatgaagcagaggggcaggaagcggagatcctggaatagcctgggcaaaatgtctcgcgaggaggccatgagcgcatacgtgaccgagatgaagctggtggcccagaaggtgatcgataccgtgcccctgagagggcggaagcgcaggtccacatttcagtgcgtggtgtccccctccgacgtggcagtggtgtggttctgggatggagccctgctgcagcccagcgaaaagtttgccatctcccagagcaggggcagaaagcggaggtcccacgtggatctgttcgacctggagagcggagagtatctgtgcccactgtgcaagagcctgtacaacacagtgatcccaatcatcccactgcagcctcagcgcggcaggaagaggagatctagcgagatcatcagcaagctgtacatccccaagaggaagatcatctctccaagaagcatcaaggacgtgctgccaccagtggaggaggccgtggatagg

SEQ ID NO: 40 - DNA, 4716 bp, synthetic construct
  source 1..4716: mol_type = other DNA
atggattggacttggattctctttctcgttgccgcagctacacgcgttcactcacctgatcggaaagccgcagtgagccactggcagcagcagtcctacctgggcagcggaatccactctggagccacaacaaccgcaccatctctgtctggccggggcaggaagaggaggtccggcgataacacaggcgagcaggtggccgtgaagtcccctaagcctgagagcggaggcaatcacatcgccgatctgaagaaggagatcgagatcctgaggagagggagaaagcggagatctaaggatcacggcgtgaactcctttctggtgtacatggccttcaaggacctgtttcagctgacagattgtcagatctacgaggtgctgtccgtcatccggagaggcagaaagcggaggtctaacacagacggcaaaatcgagttcatctctaccatggagggctacacctacccagtgtatggcgtgcagtggcatcctgagaaggcaccctacgagtggaggggccgcaagaggagatccaataatagcgtgctgcagaagcagcagctggagctgatggataccgtggacaatctggtgaacctgtgtaccaaggagggcgtgctgctgaagggaggccgcggcagaaagagacgcagcgactccaaccgggagaaggacatgggactgttcgaggtgttctctcagcacctgcccaccaccgagccagtggacagctccgtgagctctagcatcagcagaggccgcaagcggcgcagcctgaaggccgaggtgttcacagagatcctgaattacatctattccagcacagtgatcgtgaagaggcaggaaaccgtgaccgacctggccgccgccggcagaggcagaaagagacggtcccacccaaataacgtggtgtccatcaagtactgcagccactccgggctgatgtttagcgtgtccacctcctacatcaaggtgtgggatatccgcgacagcaggggcagaaaaagaaggtccacaaagaacaagcggagaaaggagtattttctggcagtgcagatccggagcctggaggaaaagatcaaggtgaagtctacagaggtggagatcctggagagaggaaggaagcgccggtccctggtgaagccacagaaggagaatgagcaggccgagaagaagaacatctctctggccttctttctgtatgacctgctgtccctgatggacagaggctttagaggacggaagaggcggtctgtggatatcgtgatcctgggcctgagctacgccaacctgcagaccggcagcggagtgctgagcgattctctgagcggcggaatgcagctgctgccagataggggcagaaagcggcgctctatcaccaaaatcaccaggagacgccacgagaatctgccccacggcgtggcaagcgtgaaagagtggttcaattacgtcaccgcaacacgcaacgaggagagaggacgcaagaggcggagcgccgtgctgaaggtgggatggctgtttcctctgtccgaggtgcctaactttacactgctgatggacggctgtggctgctggaggctgaaggaggatcagcggggccgcaagagaagatctctggtggccgccctgctgctgggctccgtgtgctgcggctccgcccagctgctgttcaataagaccaaaagcgtggagttcaccttcaggggccggaagaggcgctccagccacgtgtttggaaccaacaccagctctctggagctgttcctgatgaacggcaagatcaagggaccctgttggctggaggtgaagtctccacagctgagaggaaggaagagaaggagctctccctacgatcacacaccagggatggcaggctctctgggctaccacccctacgaggcccccctgggaagctatccatatggcgacccagcctataggaggggcagaaagcgcagatccatgacagggtcctccctgtccctggcccacctgctgatcatcagcggcctgctgtgctattccgcaaggggcaggaagcggcggtccgccgaggagggagacgagctgcagcagagatgtctggacctggagagacacctgatgctgctgagcgaggagaagcagtccctggcccaggagaatgccaggggcaggaagcgcagatctaccatgtatcacggagtgcctgtggtgggcatccctctgttcggcgaccactacgagacaatgacccgcgtgcaggccaagggaatgggcatcctgctgaggggcaggaagagacgctcccagcctgcatctcacatcctgaatagcgagaaattccctttctccaagtacgaggactgcctggagcagcagatgcccagcctgcgcgagtctccaatgcgcggcaggaagagacggtctgtggagaagagccccgcaaagaagaaggccaccaagaaggccgtgggcgccggagcagccaagagaaaggccaccggcccacccgtgagcgagctgatccgcggcagaaaacgcagatccctgctggcagccggcccatccgccgccgccgccaagccaaatatccctaaggtgctgctgccatttacaagggcaacaagagtgaactttaccctggagaggggcagaaaaaggaggagcctggcaccccaggacatgagagtgttttacgcacacaccgccagggcccagctgcccgtgtggaacaattggacccagagagcagcccgctatgagtgccggggccgcaagcggaggtcccctgcacctctgagacgccccctggcagtgctgcacgccccagtgccaacaaccgcactggcaagactgtccaggcctcagcgggcatgtccatcctctaggggcagaaagaggcgcagcaagcactggttcgtgctgacagattcctccctgaagtactacagagactctacagcagagggcgccgacgagctggatggagagatcgatctgcgcagcagaggcaggaagaggcggagcgtgaagtttctgaggaagccaggcggaaatctgggaaaggtgtatcacccaggctccatgctgtccctggcacctacaaagggcctgctgaatgagcctagaggccggaagcgcaggtctgattatcaggtgtacctgaacgccagcaaggtgcccggattcgccgatgatcctacagagctgtcctgtcgggtggtggatacaaagagcggcgaggcacgcgggaggaagaggaggagccagcgggatgatcctgtgctggagaccagccaccagaactttaggcgctccagatatcaggaggccgcatctccaagggaggccctgatccgcctgcgccggggcaggaagcggagatctcacctgctgaattatgtgtggccaaacgtgttcgagacctctccacacgtgatcgaggcagtgatgggcgccctggagggcctgagagtggccatcggcagaggcaggaaacgcaggagcagcaggacatctatggacgatctgaaggccttcacatccctgagcctgtatatgaagcctctgcgcggacgcaagcggagatcttgcgccgcctatggaggctatatcaactgcatggccgtgctgatggagagcaacgccgatcctaatatccaagacaaggagggacgcacagccctgcacaggggcagaaagagaaggtctgataaccggctgccccccaagaaggtgcccggattttgcagcttccgcgacggcctgagcttcctggtgcactgctgtaacgtgatcatcacagcccagagaggccggaagaggagatccccatactctaccgccgtgtctacctctggctgcggagagcacctggtgcacaccatcctggcaagagagtgttcccacgccctgcaggcagaggacgccagaggccgcaagagaaggtccggcgtgctgaaacagtatcagatcaagggcctggagtggctggtgtcctgtaccacaacaacccggggcagaaagaggaggtcctctaagtctccctccaagagaagcaagtcccaggaccaggcaaagaagagcaagagcccaacactgagaagaaggagccaggagaagatcggcaaggcaaggggaaggaagcggaggagcttttctctgtggaagggctttacaccctactacgcctgcctgggacctcacaccgtgctgaccttcatctttctggagcagatgaacaaggcatataagagaggccgcaagaggcgcagcgattctttcctggtggagcggggaaagctgctgcacaagcgggataacgacaaagtggatacccaggaggagaatttcctgccaaagtaccagcgggtgaggggcaggaaaagaaggagctttcggacctaccaccagatggtgctgcactctagggtgcaccgcagagcccggagagagatggactctgacggagacagggccgccagagcccgctgtagaggcaggaagcgcaggagcctggaggagaatatcaattctgccctggacatcaaggtgctgacccagagctcccagtctgccgtgaggggaggccccgggagatccaccatgcccaccagagggcggaagagacggtccggcgccagcgtgaacgcccccccagacccttgcaagcagagcccagtgagactggcagccggctctggcctggcctgcttcctgctgtggcagctgcag

SEQ ID NO: 41 - DNA, 3132 bp, synthetic construct
  source 1..3132: mol_type = other DNA
atggattggacatggattctctttctcgttgctgcagctactcgcgttcatagccagtcctatctggacagcggcatccactccggcgcaacaaccacagcacctccactgtctggaaagggaaatcctgaggaggaggatgtggatacatcccgcggcaggaagagacggtcttggatgggcgactttgtgacagcctggatggtgacagatatgatgctgcaggcaaagccctacccagactgggggaagtctgccagggccttctggaaaaggggcaggaagagacgctcccccaccggcagaccatacaatgtggataccgagtctgcacagctgtaccagggcccccacaataccctgtttggctactccgtggtgctgcactcccaccggggcagaaagaggcgcagcgagaagctggcagccgaggagcagtttcaggccctggtgaagcagatgtatcagacactgcaggacaagaccaaccagatcgacctgctgcaagccgagaggggccgcaagagaagatccaacgagaagatggattccatcgtgcagtgccgcgcactgaacatcgcagtggacctgggccagatccagtacctgttttctgacaagacaggcaccctgaggggcagaaagcggcgcagcgaggagctgcccgtggtgggacagctgctgagactgctgctgcagcacacccctctgagaacacacatgctgacaaacgccatcctggtgcagcagatcagggggaggaagaggaggtctaacctgcccctgctgcagatcgagctgctgcggtctgcaatcaaggccctgaggcctggccgcatcctggtgtacagcacatgcaccctgagcaaggcaagaggccggaagcggagaagcggatttcagtgtgccgccccactgctgatcctgaccaacaaattctgcttcctgagcacctacgtctttgtgagcaccttcctgaccggcaaggagctgagaggcaggaaacgccgctccgtgaccgcagccttcctggtgagctttatgccataccacatccagcgcaatatccacctgcactttctgcacaatgagacaaagccatgtgattctgtgaggggacggaagagaaggtctgccaggcccgagctgctgcagagccacttcatccctaccatcggccggctgcggaagaaggccggcaaggtggtgagcgaggaggagcagctgaggctgagaggcagaaagcgcagatctgaccgggtggatgcccaggacgaaaccgccgccgagttcaccctgcgggtgaacctgaccgccgccagactgggccagctggagggactgctgcaggccaggggccgcaagcgccggtccctggtgcagtctagacagaacctggtggccgaggagcgcctgtccagcggcatcctgggcacaatcggattcggacgcaagtccccactgtccaacagaagaggcaggaagagaaggagcatggcctttagaaccatctgcgtgctgctggagtatctgttcgtgctgtccgtgaggggcaggaaacggagatcctttgagaaaggacgcatctatacatttatcggagaggtggtggtgagcgtgaacccatatcagctgctgaacatctacggcagggatacaatcgagcagagaggaaggaagcggagaagctggcccgccgtgttcgcaagagtgagcgtgttcgtggattggatacacaaggtgatgaggctgggccggggacggaagaggaggtctgtgtgcgtgagctccgacaccctggatggaaattccagcagcgccggctactcttgcaacagccccgcaaaggtgtccaccaccacagattctccagtcagaggcaggaagaggagaagcgtggaggacgccgtggtgttctctacagagctggaggccagcgccatctccccaagggtggtggtgcctctggccgagacacactgtgaggagcagggcagaggcagaaagcggagatctgccgcccagccctactctaatctgtccaatctggacgtgctgaatcaggtggtgagggagagagacacaaagctgccaaagcctcagctggagcagcctcggggcagaaagagaagaagcgaggtgaagagaagccagcgcctgcgccgcggaccttcctcccctaggcgcccatatcaggagatggagtatgagaggagaggaggaagaggcgacagaagaggcagaaagagaaggagcgcatctagcctgaatgagaacgtggaccacagcgcactgctgaagctggataccagcgtgtctgagagggtgtctaggttcgattccaagcctgcccctagagggcggaagcggaggtcttgtcacccaggcccaagggacctgctgctgctgcacctgctgcagagaaaggacaatatcgaccgcggccgcaagcggagaagcgacctgctgcgcagcgtcctgcagcagagactgatcgccctgggcggcatcatcgccgcacgcatcagcgtgagaggcagaaaacgccggtcctataccatgcagaaacacctggagatgaacccccacttcggctcccaccagtatcagatcctgcccctgcactctcagatcccaagggaggagcagcggagaggccgcaagcggaggtcctgttcttcttctctgatggccctgcagaatgcctaccaggcagtgcacagcggccagtgccccgccgccatcgtgggcggcatcaacgtgctgctgaagagggggaggaagcggaggagcttcaggctgaacatccagggcctggagcggaccctgctgggcttcgtgtctaagaccctggactccgcatccgcccagtttgcagcatccgccctggtgaggggaaggaagaggcggagcatcaatcctcagggactgtccgatccctccccaatgagcgatccagtgctgacccaggatatcagccctcctgcccagggagtggaccaccggcaggtgagaggcaggaagcgccgctccctgggcccacacgaggaggatcaggtggtgtgcggattcaagaagctgtctgagtggggcctgtgttttcacccctccacagatacccacaagaagctg

SEQ ID NO: 42 - DNA, 4806 bp, synthetic construct
  source 1..4806: mol_type = other DNA
atggattggacttggattctttttctcgttgctgctgctactcgcgttcacagtcaggagaaggccttcttcgcacagctgcagctgggagaggaaaccggagagttcctgcctatccagcccgcccagcacatccagtctgagacctccggaaggggcagaaaaaggcggagctcttatctggattccggaattcactccggcgccaccaccacagcacccttcctgtctggaaaaggaaatcctgaggaggaggacgtggacacaagccagcgcggaaggaagcggcgctccggatgttgcccacccggccacggctctaggagcaggcccccagcccagcccgcctgctctagagcagagcaggatatcctgtttctgctgagaagcccaaggggcagaaagaggaggtccctggtgtccctgcagccagcccagatgcacaggttctggagagggctgtccctgcacctggactccgtggatgccgtgtatgagagaacatctgaccacagaggcaggaaaaggagaagcatccagctgccaatcatccagctgagaaaagtgtgggccgaggccgtgagatacgtgtctggcctgaaggaggattactccagactgtttcagggccaacgcggaaggaagaggagatctatctttaccggccaggcaaatgtggtgagcgagtatctgctggcctacctgttcctgtccctggtggcatttttcagactgatcgcctttctgaatctgcggggcaggaagagacggtctggcaggagatgtatccgggccacccccagagcctgcagagcaagatgtgccacatgctgcgtgcggtccagcgccgccacaagcccctgttgcggatgcagaggccgcaagaggagatccatggccttcgccctggccatcagagacaccgcaacctatgccagacagctgatgttttctaccaccctgctgatcgtgttcttcaccgtctgggtgtttcgcggacggaagcgcaggtcttggcggcccctgctgagaggccccaggctgagcctgcacaccgccgccaaggccgccgccaccgccacagagacaacatgccaggatgtggcagcaacccggggccgcaagcgccgcagcgaggccctggccgagaacccaggcctggtgaacaagagctgttatgaggtgggctggctgatcaagatgacactgtctaatcctagcgagctggacgagcgcggccgcaagagacgctccccttccggctccttcagcaccccaggctctgcaacatacggcaggtataagcccagccctgagcgctacaccgcagccccacacccactgctgtccctgaggggcagaaagcgcaggagcctgcagacaagactgcagcctgaggagagcacagagaccctggactccaactacgtggtgggccacgtgctgaatagccggaagcagaagcagctgctgcggggcagaaaaagaaggagcctggaggtgtctccctttttcaagtgtcacgaggtgatcggatcttctgtgctgttcatccacgataagaaggagcaggcaaaggtgtggatgatcgacagaggaaggaagcggcggagcgtggcacagccaaagaagaagctgggcaggagactggtgtttcacagcaacccccccagcggaaataagcagcagctgcccctggtgaatgagacaaagaggggccggaagaggaggtctgtgcacatcaggcgcaagcacagccaccagtacagctatctgtgcgaggtgtgtaagtattatacagtgacaaagggagacatggagagacactgcgccagaggacggaagcgccggtcttgtcacgtcggctctgaggagacaagggcaagagtgccagtgcacgacctgcacgtgggcatcaccaagagactgaaaacaatggaggtgctggagggcagaggcagaaagagaaggtctgtgaccgactacacctttgaggattgtcagctggcactggccgagggacacctgcgcctgcctgccgacacatgtctgctggagttcgcaagactggtgaggggccggaagcgccggagcgagatccacctggagatctttgagagcgccagcgccggcacccgcctgctgctggaccccgccaccgacccagatatcaacatcaactctatcaaggatcggggaaggaagcggagatccgtgtatgtgagcacccagttccctaagtacatccaggatatcgtggcagccacactgaagctgcctgccaacaaggtgatgtgtcacgtgagacgggtgagggggagaaagagaaggagcctgacccagagcaagagctccccaaagaggggcttctttagggaggagacagacaggctgatcaaaaacctgctgggcaagaggatctccaagctgatcagaggccggaagaggcgcagcaacaacatgatccaccaggtgccaatcaagtccctgcctcaggagtggctgtggtgcgagatgtggtgcgacgacgcatccaagaagcgggcaaagacaagaggacgcaagcgccgctctcctgagaatatcgagatcaccgtgaccctgtttaaagatcctcacgccgaggagttcgagggcaaggagtggacatttgtgatcgagaatgagtccccaaggggaaggaagagacggtccgccgcctctagaccacggctggacccttggaagctggtgtccttttctcgcaccctgaccatcagcccccctagccgcccagatacccctgagtcccctcgcgggagaaagagaagatcctccagcaagacaacatccgtgctgcagtgggccgagaagggctattacacccgctctaacaatctggtgacactggagaatggcaagcagctgaccgtgcggggcaggaagcgcagaagcctggactggcagcctagggagaatccatttcagaatctgaaggtgctgtccgtgagcgatcagcagcagaattttctggagctgtggagcgagatcctgagaggcagaaagcggcgcagcgagagatgggagtgcaccacctactggtatatcttcgtgttctgctccgcccaccctgcagtgaccgagatggccctgttcgtgacagtgttcggcctgcgcgggaggaagaggcggtccttttacaagaagtgcgcacagctgacagtgaatctgacaagattcccagagaccgtgcctggcgagctggtggtgcccgtggcaggctcctgcgtggtgagaggccgcaagagaaggtctaacaatgtggacctgatcctggccaccccaccattttctagactggagaaactgtactccggcgaggtggtgagaggcaggaagcgcaggtccaagaccatgaaggattttgtgcggaggaggtgctgggcaaggaagtataagctggtgacctctggaccttggctggaggtgcccccaatcgcactgagaaggggcagaaagcggaggagcgccaggagaatggcctttctggccaagaagggctacaggcacgattcttccacagcagtggtggggagccccaggggccacggccagtctagagagacccgcggcaggaagcggagatcttggcggcgggcacagaagctgcatttcctgcagaggggcgggctgaagcagctgagcgcccgggtgaggccagagctgaaaaccaagcagcagatcctgaggggccgcaaacggaggagctctggccggctggatcagctgctgtctctgtccatggtgtgggccaaccacgtgttcctgggatgtagctacaataaggacctgctggataaggtgatgaggggcaggaagaggcgcagcgagctgggctccctgatccgccagctgtccctgtgctctgtgaagctgtacgccgatccatctgtcccagatgtggtgatcgacatcctgcagcagatcagaggcaggaagagaagaagccctgagctgtctatcagcagcctgacatctcctatcccagagaacagcacagaaaccgaggtggccctgttcaggatcagagacagagattccggcgagcgcggccggaagcgcagaagcacctccgagctgctgggcgagggcgcctacgccaaggtgcagggcgcaatgtccctgcagaacggaaaggagtacgcagtgaagatcatcgagaagcagcgcggcaggaagagacgctctgattgggagggcaacagggcatatagccagtacgacaagttccacatcggaaacgagaagcagaactatagactgtacctgaagggccacacaggcacccggggcagaaagaggcggagcctgcggccaaagctgatcgaggagctggccgcaacccggatcgtggacgtgtccaccggagattcccactgtctggccctgtcccacgataacgaggtgagaggacggaagagaaggtcttatgcctacgacaaaagagccaacccacaggtgggagccgccttcccccacatcaagcacaactacgagtgtctggtgtatgtgcagctgccattcatgcgcggcagaaagagacgcagcaggcgctatccttctatggcccgcatccacagcatgaccatcgaggcccccatcgcaaaggacggactgcggcgcctgagcggcaatgagtacgtgtttaggggcagaaagagacggtccatctccacagcagtgaggcccaccacacccacaacctgcagcaggaggtggtggcgcccatccagcgtgggaggcagcgcctctggctccaggcctcca

SEQ ID NO: 43 - protein, 1580 aa, synthetic construct
MDWTWILFLVAAATRVHSQSYLDSGIHSGATTTAPYLSGKGNPEEEDVDTSRGRKRRSHLVSRKSISAALEHKVSALLPPAEQTGTWKLKTRGRKRRSDLRSRIEVLKRKVIEKLQHIQLLQKNVRAQLVDRGRKRRSSKDLLRQLPASNFNSLRFLIVHLKRVVDHAEENRGRKRRSTLLLDGNPFRVPQAAILMKGTAAILEYLRDRIPRGRKRRSSILESSSAFPDNAARREVESPPAVCPSDGCTWKRGRKRRSGQPYFEAFKKKMPIAFVAKGINKLLNKLFLINERGRKRRSMEDLQDMFIVHTIEEIEGLIAAHDQFKSTLPDARGRKRRSMGGQTALNCGEELFKRGVLKEYGVKVLGTSVESRGRKRRSAKGVNGSSQAPTTGKYCRLCDIQFNNLSNFITHRGRKRRSHMKLKVQGMECLDGCYVHSSIVPTFHRRCTWLLRGRKRRSQASRGEVPSGAYGYSYIPSGAYVYPPPVANGMYRGRKRRSYSSLRSIVVANYEESIKMPISEPAPGKKKSQIQRGRKRRSDLQVSAKIGFLKHHENKTYLAVGSMKTLMLNVSRGRKRRSELILPSGARVGHRSLRRYYKQRFGLSRAVAVAKRGRKRRSMLGEQGFQSLMETVDTGIVTQLQEFLQGFRGRKRRSRQIWWERLQGCQHIVEDWQKILMVRSLVVSPHERGRKRRSSTTGSNYYVRILSTIDGELLKPNASVALHKHSNRGRKRRSANELCEVNRKGCPSADPCLPYFCVQGCKLGEASRGRKRRSFEDCTSENTLIKYMTDRILLRESLREADLDHYSRGRKRRSWIRPLASGSYPSRKAGATLVVYKDLLVLFGGWTRGRKRRSSSDYSTSEMLVNVGNLPLDEFYPAVSMVALMRIRGRKRRSCESPTAHCNVLNWEQVHRLDGILSETIPIHGRGRGRKRRSNKQNKTPLDKSTSGVSEILLKTQMKMSLKCLAARGRKRRSYVCGGMFFFVSFEGKIVMHKIIYMVLFLFCVALRGRKRRSLPTPGSWWEQLTQASRAYASGGTEGFPLSRWAPRGRKRRSYSTLSLFDKYKGKSVDTIRSSVIPRHGLQSLGKRGRKRRSMNISLVWCLLVLSFAIRRWWRGRKRRSRVELQQEVEKLAHENSSMRLELDALRSKYEALQRGRKRRSSPQREPRGGQLRTPRMWPSCSRSLESLRVGAKPRGRKRRSFTLWNESTHYQILLTSFPHVENHSCFEHMHHIPRGRKRRSDFVVEAIGTEVGTLGFSIERPSQAKIECDDKGDRGRKRRSNVIDSMLFVFGYRAQKNKIRGRKRRSMMGSARVAELLLLHGAEPNCTDPATLTRPVHDARGRKRRSPPGLDKRLLPETLGPCYSNSQPVWLCLTPRQPLRGRKRRSSFLLSPNNGNLEATSNIEYAHVPHLSPAVIPTRRGRKRRSEEDEEDTYYTKDLPIHTCSYCGIHDPACVVYCNRGRKRRSLISRRDAEVVLTSRELGSLALNQSTGLPTLTLPRGRKRRSDFSSNDSSTCTMGLVLTLWSDTLIHLDGDGGFSRGRKRRSSSAAPSVMDLSSRASAALHPGIPSPNCMQSPIR

SEQ ID NO: 44 - protein, 1574 aa, synthetic construct
MDWTWILFLVAAATRVHSPDRKAAVSHWQQQSYLGSGIHSGATTTAPSLSGRGRKRRSGQSPTLLTSACFFPSAAFTAEFLFSLGSLCQHLRGRKRRSRLASAPPQKQKQKLGERLFPLIQAMHPTLAGKIRGRKRRSCGHLYAYDWISIPVVYTQVVTVAVYSFFLTCLVRGRKRRSDCMSEDGYKLDRIKGEFEFHNVTFHYPSRPEVKRGRKRRSMANDSPAKSLVDIDLPSLRDPAGIFELVEVVGNRGRKRRSWLGIRISESPEPGQRTFTTFKFCQLPFYKYSEYRGRKRRSPPLARPILPRERGAMDRIVEYLVGDGPQNRYALRGRKRRSTCVSLDDGLYECSCAPAYSGKDCQKKDGPCVINRGRKRRSLHVGLCSGPCEMAEQRCCVDYAKRGTAGCKKCKRGRKRRSSFESYLKTMPPYFLGPLKKAVRMMGAPNLIADSRGRKRRSPKFHSYVSLPFGCTRAVVEYRLLQAAYLAKPGDRGRKRRSGEYVDTGKLIDKINLPNFLKVYLNHKPPFGNTMRGRKRRSMCTNARRVRKRWLPKINSMLPEGVEMYRTVMGSRGRKRRSPDGPLLPSPGLLRLPGGPRHAVSGVPEVYPPGPRGRKRRSVGTQATKYNSEVVDKMIEEFLSSFEEKIENLTERGRKRRSTNLPIFKLKESCVRRRYSDFKWLKNELERDSKIRGRKRRSLIRWCLALRPSDRPTFEEIQKHPWMQDVLLPQERGRKRRSKFECGWCSGERRCTLHQHCTNPSSPWLDWSSHNRGRKRRSHDHTGFLTEYVATRWYSAPEIMLNSKGYTKSIDRGRKRRSSSEATEVNPESLAKECLEKSLLLLKFLPTGISSRGRKRRSVKKDRSASHLDHKLEIRQWRRSFLVSLFFCIPVRGRKRRSLFTFAVGVNICLGFTAYRIKRAEGWEEGPPTVLRGRKRRSMSQAPGAQLSPPTVYHERQRLELCARGRKRRSMSSNSFPYIEQSGGGEATELGQEATRGRKRRSQPCRKMLPDRSRAARYRANQKLVEYIVKAKGAERGRKRRSPVFHGMLERAPAEPCYRAPMEKLFYLPHVCSYTRGRKRRSTYSCVAENILGTGQRGSGAELDVQYPPKKVTTVRGRKRRSSPSATEDSLQPATDLLNRSELPQSQKAMQTKDARGRKRRSDLRKVPGTDPAFEVFPLHDHQCVLRGRKRRSDALDFKKDKGAFYLVFIWTMTRGRKRRSVDNYKQIKADLDQLRLPVEKSELWVEKSSNYENRGRKRRSNAPENGYMKCSSEGDNYGATCEFSCIGGYELQGRGRKRRSESGSSGKSSSSLVRGVHLSSSGPALLAGLVSLDRGRKRRSWTEKRASYELEFAKSTTKIAEAGKVSIQQQSHMRGRKRRSKILARKKSKRSALENSEEHSTKYSNSNNSAGSGRGRKRRSAAAAAALAAALLLLRREDPGLGAGPSMAETEALRGRKRRSLPSFPPFGSMNSNAAGSVSTQANTVQSGQLGGQRGRKRRSSGVVHRLEVDEDFEEHNAAKVHLMVHNLVPPFLRGRKRRSLQIKQQELYKNFLRFQVEEKKQREEAERERLRI

SEQ ID NO: 45 - protein, 1155 aa, synthetic construct
MDWTWILFLVAAATRVHSEQQSLSSDADTLSLTDSSVDPPIQDPRGRKRRSDQNTEDYLFQVILEKQIRIPHSLSVKAASVLKSRGRKRRSLREDGREQEIRDGGSLRMIHFGLVRGRKRRSLLQLWPVQRLTVKKSTDNLFREQKSSLNYLRQKRGRKRRSQWLRAVAVESIHRFCMQPQLLRSFCQSYDMKQHRGRKRRSLEAIVQLWRIPSFVTELYVNYDCDYYCSNLFEERGRKRRSTPPGQSPGSAPIIINIHRAQLTNPEVKYNYTEDRGRKRRSPYATVAPSTLAHPQAQVLARQQALQHAQTLAHARGRKRRSQRGLLQQIGDALSSQRGRVPPAAPPAQPRVPVTRGRKRRSMFRSKRSGLVRRLWRSGVVPDREEGGSGGGGGGRGRKRRSLEIKVSPPEGAETRKVREIGPLCGRRRPRVRKSRGRKRRSTPNFIIPAQRAEPMRIVRQPMPPPGDLEPPFQPRGRKRRSGTGALGLLLLFLLGYGLRRRPQKAEEPAPQAGGRGRKRRSDSHVSEVLQENYRLKNKLEGLISEKNELKMKSERGRKRRSVSAYQVQSPSWMQPQPCILQHPGAVLTPSMEHTRGRKRRSGRFSIVKKCIHKATRKYVAVKFVSKKMKKKEQARGRKRRSGILRDPGSEIEDRQYRIDLQSINIGTAQWHQLKRGRKRRSDGTQVLKIYVKQTESHYILVSWKVNSNVMTSNLRGRKRRSPPGYIPDELHQVARNGLFTSINSEGEFIPESMDRGRKRRSPKFRGVKMIPPGIHFLHYSSMDKANPKEVGPRMRGRKRRSNVSSAEGWHVNVTLSIRPSTGTGVMLALVSGNNRGRKRRSCQRSFRTFQALKKHLEASHLELSEADIQQLYGGRGRKRRSDSVDFRLRVSEPKAVFAKEQLACREVQAEVGASRGRKRRSDIKDGRFPYGSTQDYLKPIILIKLVQLGMAKDDRGRKRRSENYRNLAFLGIALSKPDLITFLEQGKEPWNMKQRGRKRRSWNSLGKMSREEAMSAYVTEMKLVAQKVIDTVPLRGRKRRSTFQCVVSPSDVAVVWFWDGALLQPSEKFAISQSRGRKRRSHVDLFDLESGEYLCPLCKSLYNTVIPIIPLQPQRGRKRRSSEIISKLYIPKRKIISPRSIKDVLPPVEEAVDR

SEQ ID NO: 46 - protein, 1572 aa, synthetic construct
MDWTWILFLVAAATRVHSPDRKAAVSHWQQQSYLGSGIHSGATTTAPSLSGRGRKRRSGDNTGEQVAVKSPKPESGGNHIADLKKEIEILRRGRKRRSKDHGVNSFLVYMAFKDLFQLTDCQIYEVLSVIRRGRKRRSNTDGKIEFISTMEGYTYPVYGVQWHPEKAPYEWRGRKRRSNNSVLQKQQLELMDTVDNLVNLCTKEGVLLKGGRGRKRRSDSNREKDMGLFEVFSQHLPTTEPVDSSVSSSISRGRKRRSLKAEVFTEILNYIYSSTVIVKRQETVTDLAAAGRGRKRRSHPNNVVSIKYCSHSGLMFSVSTSYIKVWDIRDSRGRKRRSTKNKRRKEYFLAVQIRSLEEKIKVKSTEVEILERGRKRRSLVKPQKENEQAEKKNISLAFFLYDLLSLMDRGFRGRKRRSVDIVILGLSYANLQTGSGVLSDSLSGGMQLLPDRGRKRRSITKITRRRHENLPHGVASVKEWFNYVTATRNEERGRKRRSAVLKVGWLFPLSEVPNFTLLMDGCGCWRLKEDQRGRKRRSLVAALLLGSVCCGSAQLLFNKTKSVEFTFRGRKRRSSHVFGTNTSSLELFLMNGKIKGPCWLEVKSPQLRGRKRRSSPYDHTPGMAGSLGYHPYEAPLGSYPYGDPAYRRGRKRRSMTGSSLSLAHLLIISGLLCYSARGRKRRSAEEGDELQQRCLDLERHLMLLSEEKQSLAQENARGRKRRSTMYHGVPVVGIPLFGDHYETMTRVQAKGMGILLRGRKRRSQPASHILNSEKFPFSKYEDCLEQQMPSLRESPMRGRKRRSVEKSPAKKKATKKAVGAGAAKRKATGPPVSELIRGRKRRSLLAAGPSAAAAKPNIPKVLLPFTRATRVNFTLERGRKRRSLAPQDMRVFYAHTARAQLPVWNNWTQRAARYECRGRKRRSPAPLRRPLAVLHAPVPTTALARLSRPQRACPSSRGRKRRSKHWFVLTDSSLKYYRDSTAEGADELDGEIDLRSRGRKRRSVKFLRKPGGNLGKVYHPGSMLSLAPTKGLLNEPRGRKRRSDYQVYLNASKVPGFADDPTELSCRVVDTKSGEARGRKRRSQRDDPVLETSHQNFRRSRYQEAASPREALIRLRRGRKRRSHLLNYVWPNVFETSPHVIEAVMGALEGLRVAIGRGRKRRSSRTSMDDLKAFTSLSLYMKPLRGRKRRSCAAYGGYINCMAVLMESNADPNIQDKEGRTALHRGRKRRSDNRLPPKKVPGFCSFRDGLSFLVHCCNVIITAQRGRKRRSPYSTAVSTSGCGEHLVHTILARECSHALQAEDARGRKRRSGVLKQYQIKGLEWLVSCTTTTRGRKRRSSKSPSKRSKSQDQAKKSKSPTLRRRSQEKIGKARGRKRRSFSLWKGFTPYYACLGPHTVLTFIFLEQMNKAYKRGRKRRSDSFLVERGKLLHKRDNDKVDTQEENFLPKYQRVRGRKRRSFRTYHQMVLHSRVHRRARREMDSDGDRAARARCRGRKRRSLEENINSALDIKVLTQSSQSAVRGGPGRSTMPTRGRKRRSGASVNAPPDPCKQSPVRLAAGSGLACFLLWQLQ

SEQ ID NO: 47 - protein, 1044 aa, synthetic construct
MDWTWILFLVAAATRVHSQSYLDSGIHSGATTTAPPLSGKGNPEEEDVDTSRGRKRRSWMGDFVTAWMVTDMMLQAKPYPDWGKSARAFWKRGRKRRSPTGRPYNVDTESAQLYQGPHNTLFGYSVVLHSHRGRKRRSEKLAAEEQFQALVKQMYQTLQDKTNQIDLLQAERGRKRRSNEKMDSIVQCRALNIAVDLGQIQYLFSDKTGTLRGRKRRSEELPVVGQLLRLLLQHTPLRTHMLTNAILVQQIRGRKRRSNLPLLQIELLRSAIKALRPGRILVYSTCTLSKARGRKRRSGFQCAAPLLILTNKFCFLSTYVFVSTFLTGKELRGRKRRSVTAAFLVSFMPYHIQRNIHLHFLHNETKPCDSVRGRKRRSARPELLQSHFIPTIGRLRKKAGKVVSEEEQLRLRGRKRRSDRVDAQDETAAEFTLRVNLTAARLGQLEGLLQARGRKRRSLVQSRQNLVAEERLSSGILGTIGFGRKSPLSNRRGRKRRSMAFRTICVLLEYLFVLSVRGRKRRSFEKGRIYTFIGEVVVSVNPYQLLNIYGRDTIEQRGRKRRSWPAVFARVSVFVDWIHKVMRLGRGRKRRSVCVSSDTLDGNSSSAGYSCNSPAKVSTTTDSPVRGRKRRSVEDAVVFSTELEASAISPRVVVPLAETHCEEQGRGRKRRSAAQPYSNLSNLDVLNQVVRERDTKLPKPQLEQPRGRKRRSEVKRSQRLRRGPSSPRRPYQEMEYERRGGRGDRRGRKRRSASSLNENVDHSALLKLDTSVSERVSRFDSKPAPRGRKRRSCHPGPRDLLLLHLLQRKDNIDRGRKRRSDLLRSVLQQRLIALGGIIAARISVRGRKRRSYTMQKHLEMNPHFGSHQYQILPLHSQIPREEQRRGRKRRSCSSSLMALQNAYQAVHSGQCPAAIVGGINVLLKRGRKRRSFRLNIQGLERTLLGFVSKTLDSASAQFAASALVRGRKRRSINPQGLSDPSPMSDPVLTQDISPPAQGVDHRQVRGRKRRSLGPHEEDQVVCGFKKLSEWGLCFHPSTDTHKKL

SEQ ID NO: 48 - protein, 1602 aa, synthetic construct
MDWTWILFLVAAATRVHSQEKAFFAQLQLGEETGEFLPIQPAQHIQSETSGRGRKRRSSYLDSGIHSGATTTAPFLSGKGNPEEEDVDTSQRGRKRRSGCCPPGHGSRSRPPAQPACSRAEQDILFLLRSPRGRKRRSLVSLQPAQMHRFWRGLSLHLDSVDAVYERTSDHRGRKRRSIQLPIIQLRKVWAEAVRYVSGLKEDYSRLFQGQRGRKRRSIFTGQANVVSEYLLAYLFLSLVAFFRLIAFLNLRGRKRRSGRRCIRATPRACRARCATCCVRSSAATSPCCGCRGRKRRSMAFALAIRDTATYARQLMFSTTLLIVFFTVWVFRGRKRRSWRPLLRGPRLSLHTAAKAAATATETTCQDVAATRGRKRRSEALAENPGLVNKSCYEVGWLIKMTLSNPSELDERGRKRRSPSGSFSTPGSATYGRYKPSPERYTAAPHPLLSLRGRKRRSLQTRLQPEESTETLDSNYVVGHVLNSRKQKQLLRGRKRRSLEVSPFFKCHEVIGSSVLFIHDKKEQAKVWMIDRGRKRRSVAQPKKKLGRRLVFHSNPPSGNKQQLPLVNETKRGRKRRSVHIRRKHSHQYSYLCEVCKYYTVTKGDMERHCARGRKRRSCHVGSEETRARVPVHDLHVGITKRLKTMEVLEGRGRKRRSVTDYTFEDCQLALAEGHLRLPADTCLLEFARLVRGRKRRSEIHLEIFESASAGTRLLLDPATDPDININSIKDRGRKRRSVYVSTQFPKYIQDIVAATLKLPANKVMCHVRRVRGRKRRSLTQSKSSPKRGFFREETDRLIKNLLGKRISKLIRGRKRRSNNMIHQVPIKSLPQEWLWCEMWCDDASKKRAKTRGRKRRSPENIEITVTLFKDPHAEEFEGKEWTFVIENESPRGRKRRSAASRPRLDPWKLVSFSRTLTISPPSRPDTPESPRGRKRRSSSKTTSVLQWAEKGYYTRSNNLVTLENGKQLTVRGRKRRSLDWQPRENPFQNLKVLSVSDQQQNFLELWSEILRGRKRRSERWECTTYWYIFVFCSAHPAVTEMALFVTVFGLRGRKRRSFYKKCAQLTVNLTRFPETVPGELVVPVAGSCVVRGRKRRSNNVDLILATPPFSRLEKLYSGEVVRGRKRRSKTMKDFVRRRCWARKYKLVTSGPWLEVPPIALRRGRKRRSARRMAFLAKKGYRHDSSTAVVGSPRGHGQSRETRGRKRRSWRRAQKLHFLQRGGLKQLSARVRPELKTKQQILRGRKRRSSGRLDQLLSLSMVWANHVFLGCSYNKDLLDKVMRGRKRRSELGSLIRQLSLCSVKLYADPSVPDVVIDILQQIRGRKRRSPELSISSLTSPIPENSTETEVALFRIRDRDSGERGRKRRSTSELLGEGAYAKVQGAMSLQNGKEYAVKIIEKQRGRKRRSDWEGNRAYSQYDKFHIGNEKQNYRLYLKGHTGTRGRKRRSLRPKLIEELAATRIVDVSTGDSHCLALSHDNEVRGRKRRSYAYDKRANPQVGAAFPHIKHNYECLVYVQLPFMRGRKRRSRRYPSMARIHSMTIEAPIAKDGLRRLSGNEYVFRGRKRRSISTAVRPTTPTTCSRRWWRPSSVGGSASGSRPP

SEQ ID NO: 49 - DNA, 7700 bp, synthetic construct
  source 1..7700: mol_type = other DNA
gctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccgccaccatggattggacatggattctctttctcgttgcagcagccacacgcgttcacagtcagtcttacctggattctggcatccacagcggagccaccaccaccgccccttatctgagcggcaagggaaacccagaggaggaggatgtggatacatccagaggccgcaagcggagatctcacctggtgtccagaaagtccatctctgccgccctggagcacaaggtgagcgccctgctgccccccgccgagcagaccggaacatggaagctgaaaaccagaggcaggaagagaagatccgacctgcggtcccgcatcgaggtgctgaagaggaaggtgatcgagaagctgcagcacatccagctgctgcagaagaatgtgcgggcacagctggtggaccggggaaggaagcggcgcagcagcaaggacctgctgcggcagctgcctgcctccaactttaactctctgcgcttcctgatcgtgcacctgaaaagagtggtggatcacgccgaggagaatcggggcagaaagcgccgctccaccctgctgctggatggcaaccctttcagagtgccccaggccgcaatcctgatgaagggcaccgccgcaatcctggagtacctgagggacagaatccctaggggcaggaagcggcggtcctccatcctggagagcagctccgcctttcccgataatgccgcccgcagggaggtggagagccccccagcagtctgtccatctgacggctgcacatggaagagaggacgcaagcggagatccgggcagccatacttcgaggcattcaagaagaagatgcccatcgcattcgtggcaaagggcatcaataagctgctgaacaagctgtttctgatcaacgagaggggccggaagcgcaggagcatggaggatctgcaggacatgtttatcgtgcacacaatcgaggagatcgagggcctgattgcagcccacgatcagttcaagagcaccctgccagacgccagaggccggaagaggcgcagcatgggcggccagaccgccctgaattgcggcgaggagctgtttaagagaggcgtgctgaaggagtacggcgtgaaggtgctgggcacctctgtggagagccgcggcaggaagcggcgctccgccaagggcgtgaacggctctagccaggcccctaccaccggaaagtactgtcgcctgtgcgacatccagtttaataatctgagcaactttatcacccacagaggacggaagcggcggagccacatgaagctgaaagtgcagggaatggagtgtctggatggctgttatgtccacagcagcatcgtgccaacattccatagaagatgcacatggctgctgcgcggcagaaaaaggagatctcaggcaagcagaggcgaggtgccaagcggcgcctacggctacagctatataccttccggagcctatgtgtaccccccacctgtggccaacggcatgtataggggcaggaagagaaggagctattccagcctgagatctatcgtggtggccaattacgaggagtccatcaagatgccaatcagcgagccagcccctggcaagaagaagtcccagatccagcgcggcaggaagaggcgctctgacctgcaggtgagcgccaagatcggcttcctgaagcaccacgagaataagacatacctggccgtgggctctatgaaaaccctgatgctgaacgtgagcaggggcagaaagcgcaggtccgagctgatcctgcctagcggcgccagggtgggccacaggagcctgaggagatactataagcagaggttcggcctgtctagagccgtggccgtggccaagcggggacgcaagaggaggtctatgctgggcgagcagggattccagtccctgatggaaaccgtggatacaggcatcgtgacacagctgcaggagtttctgcagggattcaggggccgcaagaggaggtcccgccagatctggtgggagagactgcagggctgccagcacatcgtggaggattggcagaagatcctgatggtgcgctccctggtggtgagcccccacgagagaggacggaagcgcaggtccagcaccaccggatctaactactacgtgagaatcctgtctacaatcgatggcgagctgctgaagccaaatgccagcgtggccctgcacaagcactccaacaggggccgcaagcgcaggagcgccaacgagctgtgcgaggtgaacagaaagggctgcccaagcgccgatccatgtctgccttacttctgcgtgcagggctgtaagctgggcgaggcctcccggggacgcaagcggaggtcttttgaggattgcacatctgagaacaccctgatcaagtatatgaccgatcgcatcctgctgagggagtctctgcgcgaggcagacctggaccactatagccggggaaggaaaaggagaagctggatcagaccactggcatccggcagctaccctagccgcaaggcaggagccacactggtggtgtacaaggatctgctggtgctgttcgggggctggacacgcggacgcaagagaaggagctcctccgactactccacatccgagatgctggtgaacgtgggaaacctgcccctggacgagttttatcccgccgtgagcatggtggccctgatgcgcatccggggccgcaagagaagatcctgcgagagccccacagcacactgtaatgtgctgaattgggagcaggtgcacagactggacggcatcctgtctgagacaatcccaatccacggaaggggaagggggaggaagagaaggtccaataagcagaataagaccccactggataagtctacatccggggtgtctgagatcctgctgaaaacccagatgaaaatgagcctgaagtgcctggccgcccgcggacggaagagaaggtcttacgtgtgcggcggcatgttcttctttgtgagcttcgaggggaagatcgtgatgcacaagatcatctatatggtgctgttcctgttttgtgtggcactgagaggcagaaagaggaggtccctgcctaccccagggtcttggtgggagcagctgacccaggcatctcgggcctacgcaagcggaggcaccgagggctttcctctgagccggtgggcccccaggggcagaaagagaaggtcttattccaccctgtccctgtttgacaagtacaagggcaagtccgtggataccatccggagctccgtgatcccccgccacggcctgcagtccctgggcaaaagaggcagaaagagaagaagcatgaacatctctctggtgtggtgcctgctggtgctgtccttcgccatcaggaggtggtggcgcggcaggaagcgccggagcagggtggagctgcagcaggaggtggagaagctggcacacgagaatagctccatgcggctggagctggatgccctgaggtccaaatatgaggcactgcagagggggagaaagagaaggtccagccctcagagagagccaagaggcggccagctgagaaccccaagaatgtggccttcttgttccagaagcctggagtctctgagagtgggagccaagccccggggaaggaagagacgctccttcacactgtggaacgagagcacccactatcagatcctgctgacctcttttccccacgtggagaatcactcttgttttgagcacatgcaccacatcccaagaggcagaaagcggcggtctgattttgtggtggaggccatcggaaccgaggtgggcaccctgggattctccatcgaaagaccttcccaggccaagatcgagtgcgacgacaagggcgatcggggcaggaagcgcagatccaacgtgatcgacagcatgctgttcgtgttcggatatagagcccagaagaataagatccgcggaaggaagaggcggtctatgatgggcagcgcaagggtggcagagctgctgctgctgcacggagcagagcccaactgtaccgaccctgcaacactgaccagaccagtgcacgacgcaagaggaaggaagagaagaagccccccaggcctggacaagagactgctgccagagacactgggcccctgctactccaattctcagcctgtgtggctgtgtctgacaccacggcagccactgagaggcaggaaaagaagatcttccttcctgctgagccctaataatggcaacctggaggccacatctaatatcgagtacgcacacgtgccccacctgtctcctgccgtgatccctacacggaggggccggaagaggaggtctgaggaggatgaggaggacacctattatacaaaggacctgcccatccacacctgcagctactgcggaatccacgacccagcctgcgtggtgtattgtaaccgcggcaggaagagacggagcctgatctctaggagggatgccgaggtggtgctgacaagcagggagctgggcagcctggccctgaaccagtctacaggcctgcctaccctgaccctgccaagaggccggaagcggaggtctgacttctcttctaacgactcctccacatgcaccatgggcctggtgctgacactgtggtctgataccctgatccacctggacggcgatggaggctttagcagaggcagaaaaaggaggtcctccagcgcagccccatccgtgatggacctgagctcccgggcatctgccgcactgcacccaggcatcccctcccctaattgtatgcagtctcccatccgctgataactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctt

SEQ ID NO: 50 - DNA, 7682 bp, synthetic construct
  source 1..7682: mol_type = other DNA
gctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccgccaccatggattggacctggattctctttcttgttgctgctgccactcgcgttcacagtcctgacaggaaggccgccgtgtcccactggcagcagcagagctatctgggcagcggcatccactctggcgccacaaccaccgcaccatctctgtctgggagaggcaggaaacggagaagcggccagtctcctacactgctgacatctgcctgtttcttcccatctgcagcctttacagccgagtttctgtttagcctgggctccctgtgtcagcacctgcggggccggaagagaagatctcgcctggccagcgcacctccacagaaacagaagcagaagctgggcgagaggctgttccccctgatccaggcaatgcacccaacactggccggcaaaatcaggggcagaaagaggaggtcttgcggccacctgtatgcctacgattggatctctatcccagtggtgtacacccaggtggtgaccgtggccgtgtatagcttttttctgacctgcctggtgaggggcaggaagagacgcagcgactgtatgtccgaggatggatataagctggataggatcaagggagagttcgagttccacaatgtgacctttcactatcccagccgccccgaggtgaagagaggaaggaagcggaggtctatggccaacgattcccctgccaagtccctggtggacatcgacctgccttccctgagggacccagccggcatcttcgagctggtggaggtggtgggaaataggggccgcaagagacgcagctggctgggcatccggatctctgagtctccagagccagggcagcggacattcacaacctttaagttttgccagctgccattctacaagtattccgagtatagaggcagaaagcggaggagcccccccctggcaagaccaatcctgccaagggagcgcggcgcaatggatagaatcgtggaatacctggtgggagacggccctcagaacagatacgcactgagaggccggaaaaggagaagcacctgcgtgtccctggatgacgggctgtacgagtgtagctgtgcccctgcctattccggaaaggattgtcagaagaaggatggaccttgcgtgatcaacaggggcaggaagcgcagaagcctgcacgtgggcctgtgttccggcccctgtgagatggccgagcagcggtgctgcgtggactacgccaagaggggcaccgccgggtgtaagaagtgtaagaggggccggaagcggcgctccagcttcgagagctacctgaaaacaatgccaccatactttctgggcccactgaagaaggcagtgcgcatgatgggagccccaaacctgatcgcagacagcaggggcagaaagcgccgcagccctaagttccactcctacgtgtctctgccattcggctgcacaagggccgtggtggagtacaggctgctgcaggccgcatacctggccaagcccggcgacagaggccggaagcgccgctccggagagtacgtggatacaggcaagctgatcgataagatcaatctgcctaatttcctgaaggtgtatctgaaccacaagccaccttttggcaacaccatgcggggcaggaaaagaagatctatgtgcaccaacgccagaagggtgagaaagagatggctgcctaagatcaacagcatgctgcccgagggagtggagatgtaccgcaccgtgatgggatctagagggagaaagagaagaagcccagacggcccactgctgccatctcccggcctgctgagactgccaggcgggcctcggcacgccgtgtctggagtgcctgaggtgtacccccctggccctagaggcaggaagcggcggagcgtgggcacacaggccaccaagtataatagcgaggtggtggacaagatgatcgaggagtttctgtccagctttgaggagaagatcgagaatctgacagagaggggacggaagcgccggagcacaaacctgcctatctttaagctgaaggagagctgcgtgcgcaggagatactctgatttcaagtggctgaagaatgagctggagagagactctaagatcagaggcaggaaaaggcgctccctgatcagatggtgtctggcactgcgcccaagcgacaggccaaccttcgaggagatccagaagcacccatggatgcaggacgtgctgctgccccaggagcggggccggaagcgcagatccaagtttgaatgcggctggtgtagcggcgagaggaggtgtacactgcaccagcactgcacaaacccaagctccccatggctggattggtccagccacaacaggggacggaagagacggagccacgaccacaccggcttcctgaccgagtacgtggccacaaggtggtacagcgcacccgagatcatgctgaattccaaaggctacacaaagtctatcgataggggaaggaagcgccgcagctcctccgaggcaaccgaggtgaatccagagtctctggcaaaggagtgcctggagaaatctctgctgctgctgaagttcctgcccaccggcatcagctccaggggaaggaagaggagaagcgtgaagaaggatagatccgcctctcacctggaccacaaactggagatccgccagtggaggaggtcctttctggtgtccctgtttttctgcatccccgtgcgcggacgcaagcgcaggagcctgtttacattcgccgtgggagtgaatatctgcctgggctttaccgcctacaggatcaagagagcagagggatgggaggagggccctcccaccgtgctgcggggcaggaagaggagatccatgagccaggcccctggcgcacagctgtctcccccaaccgtgtaccacgagagacagaggctggagctgtgtgcaaggggcagaaagcggcgctccatgtcttctaacagctttccctacatcgagcagtccggaggaggcgaggccaccgagctgggccaggaggccaccaggggaaggaaaagaaggtcccagccttgtcgcaagatgctgcctgatcggtccagagccgccagatacagggccaatcagaagctggtggagtatatcgtgaaggcaaagggagccgagaggggccgcaagcggagaagccctgtctttcacggcatgctggagagagcaccagccgagccttgctatcgggccccaatggagaagctgttttatctgccacacgtgtgctcttatacccggggccgcaagcggcgctccacctactcttgcgtggccgagaatatcctgggcacaggacagagaggctccggcgccgagctggatgtgcagtatccccccaagaaggtgaccaccgtgagaggcaggaagcgccgctccagccccagcgcaacagaggattccctgcagcctgccacagacctgctgaacaggtctgagctgccccagagccagaaggcaatgcagaccaaggatgcccgcggcagaaagagaaggtctgacctgagaaaggtgcccggcacagaccccgccttcgaggtgtttcccctgcacgatcaccagtgtgtgctgcggggccgcaagcgccggagcgacgcactggatttcaagaaggataagggcgccttctatctggtgttcatctggaccatgaccagaggcaggaagagaagatccgtggataactacaagcagatcaaggccgacctggatcagctgaggctgcctgtcgagaagtccgagctgtgggtggagaagagctctaactatgagaataggggcagaaagagaagatccaatgcccccgagaatggatatatgaagtgctccagcgagggcgacaactacggcgccacctgcgagttctcttgcatcggaggctatgagctgcagggccggggccgcaagagaagatccgagagcggaagcagcggcaagagctcctcttccctggtgcggggagtccacctgagcagctccggacccgccctgctggccggcctggtgtccctggacaggggccggaagaggagatcttggacagagaagcgggcctcttatgagctggagttcgccaagtccacaacaaagatcgccgaggccggcaaggtgtccatccagcaacagtctcacatgagaggcagaaagcgcaggagcaagatcctggcaaggaagaagtccaaacggtctgcactggagaactccgaggagcactccacaaagtactctaattccaacaactctgccggctccgggagaggcagaaaaagaaggtccgcagcagcagccgccgcactggccgcagccctgctgctgctgaggcgcgaggacccaggactgggggccggccccagcatggcagagacagaggccctgcgcggcagaaagaggagatctctgccttcttttcctcctttcggctctatgaactctaacgccgcaggcagcgtgagcacccaggccaataccgtgcagagcggacagctgggaggacagagaggcagaaagagacggtccagcggagtggtgcaccggctggaggtggacgaggacttcgaggagcacaatgccgcaaaggtgcatctgatggtgcacaatctggtgccaccattcctgaggggcaggaagcggaggagcctgcagatcaagcagcaggagctgtataagaacttcctgagattccaggtggaggagaagaagcagcgggaggaggccgagcgcgagcgcctgagaatctgataactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctt

SEQ ID NO: 51 - DNA, 6425 bp, synthetic construct
  source 1..6425: mol_type = other DNA
gctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccgccaccatggattggacatggattctttttctcgttgcagcagctacacgcgttcatagcgagcagcagtctctgtctagcgacgccgataccctgtctctgaccgactctagcgtggacccacctatccaggacccaaggggcagaaaaaggagatctgaccagaacaccgaggattacctgtttcaggtgatcctggagaagcagatcagaatcccccacagcctgagcgtgaaggccgcctccgtgctgaagtctagaggccggaagagaaggagcctgagagaggatggccgggagcaggagatccgcgacggcggctccctgcggatgatccacttcggcctggtgagaggccgcaagagacggagcctgctgcagctgtggccagtgcagcggctgacagtgaagaagtccaccgacaatctgttcagagagcagaaatcctccctgaactacctgcgccagaagcggggcagaaagcgccggtcccagtggctgagggccgtggcagtggagagcatccaccgcttctgtatgcagccacagctgctgagatccttctgtcagagctatgatatgaagcagcacagaggaaggaagcggaggagcctggaggccatcgtgcagctgtggcgcatcccatccttcgtgaccgagctgtatgtgaattatgattgtgattattattgctccaatctgttcgaggagagggggagaaagcgcagatccacccctcccggccagtccccaggctccgcccccatcatcatcaacatccacagagcccagctgacaaacccagaggtgaagtacaattataccgaggacaggggacggaagagacgctctccttatgccacagtggcaccctccaccctggcccaccctcaggcccaggtgctggccaggcagcaggccctgcagcacgcccagaccctggcccacgccagaggcagaaaacggagaagccagaggggcctgctgcagcagatcggcgacgcactgtccagccagaggggacgggtccccccagccgcaccccctgcacagcccagggtgccagtgaccagaggcagaaagcgcaggtccatgtttaggtctaagagatccggcctggtgaggaggctgtggcggtccggcgtggtgcccgatcgcgaggagggaggaagcggcggcggcgggggaggccgcggcagaaagaggcggtccctggagatcaaggtgagcccacccgagggcgcagagacacgcaaggtgagagagatcggccctctgtgcggacgcagaaggccacgggtgagaaagtccaggggcaggaagagaaggtctacccctaactttatcatcccagcccagagagccgagcctatgaggatcgtgcgccagcctatgccaccccccggggacctggagcctcctttccagcctagaggcagaaagcggcgctctggcacaggcgccctgggcctgctgctgctgttcctgctgggctatggactgaggagaaggcctcagaaggccgaggagcctgcacctcaggcaggcggccggggacggaagagaagatctgacagccacgtgtccgaggtgctgcaggagaactatcggctgaaaaacaagctggagggactgatcagcgagaagaatgagctgaagatgaagagcgagaggggacggaagcggagatctgtgtctgcataccaagtgcagagcccaagctggatgcagcctcagccatgtatcctgcagcaccctggcgccgtgctgaccccttctatggagcacaccaggggccggaaacggaggagcggcaggttcagcatcgtgaagaagtgcatccacaaggcaaccaggaagtacgtggccgtgaagttcgtgtccaagaagatgaaaaagaaggagcaggcccgcggacgcaagcgccggtctggcatcctgcgcgacccaggctctgagatcgaggacagacagtatagaatcgatctgcagtccatcaatatcggaaccgcccagtggcaccagctgaagagaggaaggaagagaaggtccgacggaacacaggtgctgaagatctacgtgaagcagacagagtcccactacatcctggtgtcttggaaggtgaattctaacgtgatgacatctaatctgagaggccgcaagcgccggagccccccaggatacatccccgatgagctgcaccaggtggcccggaacggcctgtttacatctatcaactctgagggcgagttcatccccgagagcatggataggggcagaaagagacggagcccaaagtttcggggagtgaaaatgatccctccaggcattcacttcctgcactatagcagcatggacaaagccaatcctaaggaggtgggcccacgcatgagaggcagaaagagaagatccaatgtgagcagcgccgagggatggcacgtgaacgtgaccctgtctatcaggccctccacaggcaccggcgtgatgctggcactggtgagcggaaataacaggggccgcaagaggagatcttgtcagaggtcttttaggacctttcaggccctgaagaagcacctggaggcctcccacctggagctgagcgaggccgatatccagcagctgtacggcggccgcggcaggaagcgcagatccgactccgtggactttcgcctgagggtgagcgagcccaaggcagtgttcgccaaggagcagctggcatgcagggaggtgcaggcagaggtgggcgcctctagaggcaggaagaggcgcagcgatatcaaggatggaagatttccttacggatctacccaggattacctgaagccaatcatcctgatcaagctggtgcagctgggcatggcaaaggatgacaggggcagaaagaggcgctccgagaactaccggaacctggcctttctggggatcgcactgtctaagccagacctgatcacattcctggagcagggcaaggagccctggaatatgaagcagaggggcaggaagcggagatcctggaatagcctgggcaaaatgtctcgcgaggaggccatgagcgcatacgtgaccgagatgaagctggtggcccagaaggtgatcgataccgtgcccctgagagggcggaagcgcaggtccacatttcagtgcgtggtgtccccctccgacgtggcagtggtgtggttctgggatggagccctgctgcagcccagcgaaaagtttgccatctcccagagcaggggcagaaagcggaggtcccacgtggatctgttcgacctggagagcggagagtatctgtgcccactgtgcaagagcctgtacaacacagtgatcccaatcatcccactgcagcctcagcgcggcaggaagaggagatctagcgagatcatcagcaagctgtacatccccaagaggaagatcatctctccaagaagcatcaaggacgtgctgccaccagtggaggaggccgtggataggtgataactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctt

SEQ ID NO: 52 - DNA, 7676 bp, synthetic construct
  source 1..7676: mol_type = other DNA
gctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccgccaccatggattggacttggattctctttctcgttgccgcagctacacgcgttcactcacctgatcggaaagccgcagtgagccactggcagcagcagtcctacctgggcagcggaatccactctggagccacaacaaccgcaccatctctgtctggccggggcaggaagaggaggtccggcgataacacaggcgagcaggtggccgtgaagtcccctaagcctgagagcggaggcaatcacatcgccgatctgaagaaggagatcgagatcctgaggagagggagaaagcggagatctaaggatcacggcgtgaactcctttctggtgtacatggccttcaaggacctgtttcagctgacagattgtcagatctacgaggtgctgtccgtcatccggagaggcagaaagcggaggtctaacacagacggcaaaatcgagttcatctctaccatggagggctacacctacccagtgtatggcgtgcagtggcatcctgagaaggcaccctacgagtggaggggccgcaagaggagatccaataatagcgtgctgcagaagcagcagctggagctgatggataccgtggacaatctggtgaacctgtgtaccaaggagggcgtgctgctgaagggaggccgcggcagaaagagacgcagcgactccaaccgggagaaggacatgggactgttcgaggtgttctctcagcacctgcccaccaccgagccagtggacagctccgtgagctctagcatcagcagaggccgcaagcggcgcagcctgaaggccgaggtgttcacagagatcctgaattacatctattccagcacagtgatcgtgaagaggcaggaaaccgtgaccgacctggccgccgccggcagaggcagaaagagacggtcccacccaaataacgtggtgtccatcaagtactgcagccactccgggctgatgtttagcgtgtccacctcctacatcaaggtgtgggatatccgcgacagcaggggcagaaaaagaaggtccacaaagaacaagcggagaaaggagtattttctggcagtgcagatccggagcctggaggaaaagatcaaggtgaagtctacagaggtggagatcctggagagaggaaggaagcgccggtccctggtgaagccacagaaggagaatgagcaggccgagaagaagaacatctctctggccttctttctgtatgacctgctgtccctgatggacagaggctttagaggacggaagaggcggtctgtggatatcgtgatcctgggcctgagctacgccaacctgcagaccggcagcggagtgctgagcgattctctgagcggcggaatgcagctgctgccagataggggcagaaagcggcgctctatcaccaaaatcaccaggagacgccacgagaatctgccccacggcgtggcaagcgtgaaagagtggttcaattacgtcaccgcaacacgcaacgaggagagaggacgcaagaggcggagcgccgtgctgaaggtgggatggctgtttcctctgtccgaggtgcctaactttacactgctgatggacggctgtggctgctggaggctgaaggaggatcagcggggccgcaagagaagatctctggtggccgccctgctgctgggctccgtgtgctgcggctccgcccagctgctgttcaataagaccaaaagcgtggagttcaccttcaggggccggaagaggcgctccagccacgtgtttggaaccaacaccagctctctggagctgttcctgatgaacggcaagatcaagggaccctgttggctggaggtgaagtctccacagctgagaggaaggaagagaaggagctctccctacgatcacacaccagggatggcaggctctctgggctaccacccctacgaggcccccctgggaagctatccatatggcgacccagcctataggaggggcagaaagcgcagatccatgacagggtcctccctgtccctggcccacctgctgatcatcagcggcctgctgtgctattccgcaaggggcaggaagcggcggtccgccgaggagggagacgagctgcagcagagatgtctggacctggagagacacctgatgctgctgagcgaggagaagcagtccctggcccaggagaatgccaggggcaggaagcgcagatctaccatgtatcacggagtgcctgtggtgggcatccctctgttcggcgaccactacgagacaatgacccgcgtgcaggccaagggaatgggcatcctgctgaggggcaggaagagacgctcccagcctgcatctcacatcctgaatagcgagaaattccctttctccaagtacgaggactgcctggagcagcagatgcccagcctgcgcgagtctccaatgcgcggcaggaagagacggtctgtggagaagagccccgcaaagaagaaggccaccaagaaggccgtgggcgccggagcagccaagagaaaggccaccggcccacccgtgagcgagctgatccgcggcagaaaacgcagatccctgctggcagccggcccatccgccgccgccgccaagccaaatatccctaaggtgctgctgccatttacaagggcaacaagagtgaactttaccctggagaggggcagaaaaaggaggagcctggcaccccaggacatgagagtgttttacgcacacaccgccagggcccagctgcccgtgtggaacaattggacccagagagcagcccgctatgagtgccggggccgcaagcggaggtcccctgcacctctgagacgccccctggcagtgctgcacgccccagtgccaacaaccgcactggcaagactgtccaggcctcagcgggcatgtccatcctctaggggcagaaagaggcgcagcaagcactggttcgtgctgacagattcctccctgaagtactacagagactctacagcagagggcgccgacgagctggatggagagatcgatctgcgcagcagaggcaggaagaggcggagcgtgaagtttctgaggaagccaggcggaaatctgggaaaggtgtatcacccaggctccatgctgtccctggcacctacaaagggcctgctgaatgagcctagaggccggaagcgcaggtctgattatcaggtgtacctgaacgccagcaaggtgcccggattcgccgatgatcctacagagctgtcctgtcgggtggtggatacaaagagcggcgaggcacgcgggaggaagaggaggagccagcgggatgatcctgtgctggagaccagccaccagaactttaggcgctccagatatcaggaggccgcatctccaagggaggccctgatccgcctgcgccggggcaggaagcggagatctcacctgctgaattatgtgtggccaaacgtgttcgagacctctccacacgtgatcgaggcagtgatgggcgccctggagggcctgagagtggccatcggcagaggcaggaaacgcaggagcagcaggacatctatggacgatctgaaggccttcacatccctgagcctgtatatgaagcctctgcgcggacgcaagcggagatcttgcgccgcctatggaggctatatcaactgcatggccgtgctgatggagagcaacgccgatcctaatatccaagacaaggagggacgcacagccctgcacaggggcagaaagagaaggtctgataaccggctgccccccaagaaggtgcccggattttgcagcttccgcgacggcctgagcttcctggtgcactgctgtaacgtgatcatcacagcccagagaggccggaagaggagatccccatactctaccgccgtgtctacctctggctgcggagagcacctggtgcacaccatcctggcaagagagtgttcccacgccctgcaggcagaggacgccagaggccgcaagagaaggtccggcgtgctgaaacagtatcagatcaagggcctggagtggctggtgtcctgtaccacaacaacccggggcagaaagaggaggtcctctaagtctccctccaagagaagcaagtcccaggaccaggcaaagaagagcaagagcccaacactgagaagaaggagccaggagaagatcggcaaggcaaggggaaggaagcggaggagcttttctctgtggaagggctttacaccctactacgcctgcctgggacctcacaccgtgctgaccttcatctttctggagcagatgaacaaggcatataagagaggccgcaagaggcgcagcgattctttcctggtggagcggggaaagctgctgcacaagcgggataacgacaaagtggatacccaggaggagaatttcctgccaaagtaccagcgggtgaggggcaggaaaagaaggagctttcggacctaccaccagatggtgctgcactctagggtgcaccgcagagcccggagagagatggactctgacggagacagggccgccagagcccgctgtagaggcaggaagcgcaggagcctggaggagaatatcaattctgccctggacatcaaggtgctgacccagagctcccagtctgccgtgaggggaggccccgggagatccaccatgcccaccagagggcggaagagacggtccggcgccagcgtgaacgcccccccagacccttgcaagcagagcccagtgagactggcagccggctctggcctggcctgcttcctgctgtggcagctgcagtgataactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctt

SEQ ID NO: 53 - DNA, 6092 bp, synthetic construct
  source 1..6092: mol_type = other DNA
gctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccgccaccatggattggacatggattctctttctcgttgctgcagctactcgcgttcatagccagtcctatctggacagcggcatccactccggcgcaacaaccacagcacctccactgtctggaaagggaaatcctgaggaggaggatgtggatacatcccgcggcaggaagagacggtcttggatgggcgactttgtgacagcctggatggtgacagatatgatgctgcaggcaaagccctacccagactgggggaagtctgccagggccttctggaaaaggggcaggaagagacgctcccccaccggcagaccatacaatgtggataccgagtctgcacagctgtaccagggcccccacaataccctgtttggctactccgtggtgctgcactcccaccggggcagaaagaggcgcagcgagaagctggcagccgaggagcagtttcaggccctggtgaagcagatgtatcagacactgcaggacaagaccaaccagatcgacctgctgcaagccgagaggggccgcaagagaagatccaacgagaagatggattccatcgtgcagtgccgcgcactgaacatcgcagtggacctgggccagatccagtacctgttttctgacaagacaggcaccctgaggggcagaaagcggcgcagcgaggagctgcccgtggtgggacagctgctgagactgctgctgcagcacacccctctgagaacacacatgctgacaaacgccatcctggtgcagcagatcagggggaggaagaggaggtctaacctgcccctgctgcagatcgagctgctgcggtctgcaatcaaggccctgaggcctggccgcatcctggtgtacagcacatgcaccctgagcaaggcaagaggccggaagcggagaagcggatttcagtgtgccgccccactgctgatcctgaccaacaaattctgcttcctgagcacctacgtctttgtgagcaccttcctgaccggcaaggagctgagaggcaggaaacgccgctccgtgaccgcagccttcctggtgagctttatgccataccacatccagcgcaatatccacctgcactttctgcacaatgagacaaagccatgtgattctgtgaggggacggaagagaaggtctgccaggcccgagctgctgcagagccacttcatccctaccatcggccggctgcggaagaaggccggcaaggtggtgagcgaggaggagcagctgaggctgagaggcagaaagcgcagatctgaccgggtggatgcccaggacgaaaccgccgccgagttcaccctgcgggtgaacctgaccgccgccagactgggccagctggagggactgctgcaggccaggggccgcaagcgccggtccctggtgcagtctagacagaacctggtggccgaggagcgcctgtccagcggcatcctgggcacaatcggattcggacgcaagtccccactgtccaacagaagaggcaggaagagaaggagcatggcctttagaaccatctgcgtgctgctggagtatctgttcgtgctgtccgtgaggggcaggaaacggagatcctttgagaaaggacgcatctatacatttatcggagaggtggtggtgagcgtgaacccatatcagctgctgaacatctacggcagggatacaatcgagcagagaggaaggaagcggagaagctggcccgccgtgttcgcaagagtgagcgtgttcgtggattggatacacaaggtgatgaggctgggccggggacggaagaggaggtctgtgtgcgtgagctccgacaccctggatggaaattccagcagcgccggctactcttgcaacagccccgcaaaggtgtccaccaccacagattctccagtcagaggcaggaagaggagaagcgtggaggacgccgtggtgttctctacagagctggaggccagcgccatctccccaagggtggtggtgcctctggccgagacacactgtgaggagcagggcagaggcagaaagcggagatctgccgcccagccctactctaatctgtccaatctggacgtgctgaatcaggtggtgagggagagagacacaaagctgccaaagcctcagctggagcagcctcggggcagaaagagaagaagcgaggtgaagagaagccagcgcctgcgccgcggaccttcctcccctaggcgcccatatcaggagatggagtatgagaggagaggaggaagaggcgacagaagaggcagaaagagaaggagcgcatctagcctgaatgagaacgtggaccacagcgcactgctgaagctggataccagcgtgtctgagagggtgtctaggttcgattccaagcctgcccctagagggcggaagcggaggtcttgtcacccaggcccaagggacctgctgctgctgcacctgctgcagagaaaggacaatatcgaccgcggccgcaagcggagaagcgacctgctgcgcagcgtcctgcagcagagactgatcgccctgggcggcatcatcgccgcacgcatcagcgtgagaggcagaaaacgccggtcctataccatgcagaaacacctggagatgaacccccacttcggctcccaccagtatcagatcctgcccctgcactctcagatcccaagggaggagcagcggagaggccgcaagcggaggtcctgttcttcttctctgatggccctgcagaatgcctaccaggcagtgcacagcggccagtgccccgccgccatcgtgggcggcatcaacgtgctgctgaagagggggaggaagcggaggagcttcaggctgaacatccagggcctggagcggaccctgctgggcttcgtgtctaagaccctggactccgcatccgcccagtttgcagcatccgccctggtgaggggaaggaagaggcggagcatcaatcctcagggactgtccgatccctccccaatgagcgatccagtgctgacccaggatatcagccctcctgcccagggagtggaccaccggcaggtgagaggcaggaagcgccgctccctgggcccacacgaggaggatcaggtggtgtgcggattcaagaagctgtctgagtggggcctgtgttttcacccctccacagatacccacaagaagctgtgataactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctt

SEQ ID NO: 54 - DNA, 7766 bp, synthetic construct
  source 1..7766: mol_type = other DNA
gctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccgccaccatggattggacttggattctttttctcgttgctgctgctactcgcgttcacagtcaggagaaggccttcttcgcacagctgcagctgggagaggaaaccggagagttcctgcctatccagcccgcccagcacatccagtctgagacctccggaaggggcagaaaaaggcggagctcttatctggattccggaattcactccggcgccaccaccacagcacccttcctgtctggaaaaggaaatcctgaggaggaggacgtggacacaagccagcgcggaaggaagcggcgctccggatgttgcccacccggccacggctctaggagcaggcccccagcccagcccgcctgctctagagcagagcaggatatcctgtttctgctgagaagcccaaggggcagaaagaggaggtccctggtgtccctgcagccagcccagatgcacaggttctggagagggctgtccctgcacctggactccgtggatgccgtgtatgagagaacatctgaccacagaggcaggaaaaggagaagcatccagctgccaatcatccagctgagaaaagtgtgggccgaggccgtgagatacgtgtctggcctgaaggaggattactccagactgtttcagggccaacgcggaaggaagaggagatctatctttaccggccaggcaaatgtggtgagcgagtatctgctggcctacctgttcctgtccctggtggcatttttcagactgatcgcctttctgaatctgcggggcaggaagagacggtctggcaggagatgtatccgggccacccccagagcctgcagagcaagatgtgccacatgctgcgtgcggtccagcgccgccacaagcccctgttgcggatgcagaggccgcaagaggagatccatggccttcgccctggccatcagagacaccgcaacctatgccagacagctgatgttttctaccaccctgctgatcgtgttcttcaccgtctgggtgtttcgcggacggaagcgcaggtcttggcggcccctgctgagaggccccaggctgagcctgcacaccgccgccaaggccgccgccaccgccacagagacaacatgccaggatgtggcagcaacccggggccgcaagcgccgcagcgaggccctggccgagaacccaggcctggtgaacaagagctgttatgaggtgggctggctgatcaagatgacactgtctaatcctagcgagctggacgagcgcggccgcaagagacgctccccttccggctccttcagcaccccaggctctgcaacatacggcaggtataagcccagccctgagcgctacaccgcagccccacacccactgctgtccctgaggggcagaaagcgcaggagcctgcagacaagactgcagcctgaggagagcacagagaccctggactccaactacgtggtgggccacgtgctgaatagccggaagcagaagcagctgctgcggggcagaaaaagaaggagcctggaggtgtctccctttttcaagtgtcacgaggtgatcggatcttctgtgctgttcatccacgataagaaggagcaggcaaaggtgtggatgatcgacagaggaaggaagcggcggagcgtggcacagccaaagaagaagctgggcaggagactggtgtttcacagcaacccccccagcggaaataagcagcagctgcccctggtgaatgagacaaagaggggccggaagaggaggtctgtgcacatcaggcgcaagcacagccaccagtacagctatctgtgcgaggtgtgtaagtattatacagtgacaaagggagacatggagagacactgcgccagaggacggaagcgccggtcttgtcacgtcggctctgaggagacaagggcaagagtgccagtgcacgacctgcacgtgggcatcaccaagagactgaaaacaatggaggtgctggagggcagaggcagaaagagaaggtctgtgaccgactacacctttgaggattgtcagctggcactggccgagggacacctgcgcctgcctgccgacacatgtctgctggagttcgcaagactggtgaggggccggaagcgccggagcgagatccacctggagatctttgagagcgccagcgccggcacccgcctgctgctggaccccgccaccgacccagatatcaacatcaactctatcaaggatcggggaaggaagcggagatccgtgtatgtgagcacccagttccctaagtacatccaggatatcgtggcagccacactgaagctgcctgccaacaaggtgatgtgtcacgtgagacgggtgagggggagaaagagaaggagcctgacccagagcaagagctccccaaagaggggcttctttagggaggagacagacaggctgatcaaaaacctgctgggcaagaggatctccaagctgatcagaggccggaagaggcgcagcaacaacatgatccaccaggtgccaatcaagtccctgcctcaggagtggctgtggtgcgagatgtggtgcgacgacgcatccaagaagcgggcaaagacaagaggacgcaagcgccgctctcctgagaatatcgagatcaccgtgaccctgtttaaagatcctcacgccgaggagttcgagggcaaggagtggacatttgtgatcgagaatgagtccccaaggggaaggaagagacggtccgccgcctctagaccacggctggacccttggaagctggtgtccttttctcgcaccctgaccatcagcccccctagccgcccagatacccctgagtcccctcgcgggagaaagagaagatcctccagcaagacaacatccgtgctgcagtgggccgagaagggctattacacccgctctaacaatctggtgacactggagaatggcaagcagctgaccgtgcggggcaggaagcgcagaagcctggactggcagcctagggagaatccatttcagaatctgaaggtgctgtccgtgagcgatcagcagcagaattttctggagctgtggagcgagatcctgagaggcagaaagcggcgcagcgagagatgggagtgcaccacctactggtatatcttcgtgttctgctccgcccaccctgcagtgaccgagatggccctgttcgtgacagtgttcggcctgcgcgggaggaagaggcggtccttttacaagaagtgcgcacagctgacagtgaatctgacaagattcccagagaccgtgcctggcgagctggtggtgcccgtggcaggctcctgcgtggtgagaggccgcaagagaaggtctaacaatgtggacctgatcctggccaccccaccattttctagactggagaaactgtactccggcgaggtggtgagaggcaggaagcgcaggtccaagaccatgaaggattttgtgcggaggaggtgctgggcaaggaagtataagctggtgacctctggaccttggctggaggtgcccccaatcgcactgagaaggggcagaaagcggaggagcgccaggagaatggcctttctggccaagaagggctacaggcacgattcttccacagcagtggtggggagccccaggggccacggccagtctagagagacccgcggcaggaagcggagatcttggcggcgggcacagaagctgcatttcctgcagaggggcgggctgaagcagctgagcgcccgggtgaggccagagctgaaaaccaagcagcagatcctgaggggccgcaaacggaggagctctggccggctggatcagctgctgtctctgtccatggtgtgggccaaccacgtgttcctgggatgtagctacaataaggacctgctggataaggtgatgaggggcaggaagaggcgcagcgagctgggctccctgatccgccagctgtccctgtgctctgtgaagctgtacgccgatccatctgtcccagatgtggtgatcgacatcctgcagcagatcagaggcaggaagagaagaagccctgagctgtctatcagcagcctgacatctcctatcccagagaacagcacagaaaccgaggtggccctgttcaggatcagagacagagattccggcgagcgcggccggaagcgcagaagcacctccgagctgctgggcgagggcgcctacgccaaggtgcagggcgcaatgtccctgcagaacggaaaggagtacgcagtgaagatcatcgagaagcagcgcggcaggaagagacgctctgattgggagggcaacagggcatatagccagtacgacaagttccacatcggaaacgagaagcagaactatagactgtacctgaagggccacacaggcacccggggcagaaagaggcggagcctgcggccaaagctgatcgaggagctggccgcaacccggatcgtggacgtgtccaccggagattcccactgtctggccctgtcccacgataacgaggtgagaggacggaagagaaggtcttatgcctacgacaaaagagccaacccacaggtgggagccgccttcccccacatcaagcacaactacgagtgtctggtgtatgtgcagctgccattcatgcgcggcagaaagagacgcagcaggcgctatccttctatggcccgcatccacagcatgaccatcgaggcccccatcgcaaaggacggactgcggcgcctgagcggcaatgagtacgtgtttaggggcagaaagagacggtccatctccacagcagtgaggcccaccacacccacaacctgcagcaggaggtggtggcgcccatccagcgtgggaggcagcgcctctggctccaggcctccatgataactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctt

SEQ ID NO: 55 - protein, 16 aa, synthetic construct
MDWTWILFLVAAATRV

SEQ ID NO: 56 - protein, 219 aa, Homo sapiens
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 57 - protein, 328 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 58 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 59 - protein, 155 aa, Homo sapiens
MTPGKTSLVSLLLLLSLEAIVKAGITIPRNPGCPNSEDKNFPRTVMVNLNIHNRNTNTNPKRSSDYYNRSTSPWNLHRNEDPERYPSVIWEAKCRHLGCINADGNVDYHMNSVPIQQEILVLRREPPHCPNSFRLEKILVSVGCTCVTPIVHHVA

SEQ ID NO: 60 - protein, 99 aa, Homo sapiens
MTSKLAVALLAAFLISAALCEGAVLPRSAKELRCQCIKTYSKPFHPKFIKELRVIESGPHCANTEIIVKLSDGRELCLDPKENWVQRVVEKFLKRAENS

SEQ ID NO: 61 - protein, 91 aa, Homo sapiens
MKVSAAALAVILIATALCAPASASPYSSDTTPCCFAYIARPLPRAHIKEYFYTSGKCSNPAVVFVTRKNRQVCANPEKKWVREYINSLEMS

SEQ ID NO: 62 - protein, 92 aa, Homo sapiens
MQVSTAALAVLLCTMALCNQFSASLAADTPTACCFSYTSRQIPQNFIADYFETSSQCSKPGVIFLTKRSRQVCADPSEEWVQKYVSDLELSA

SEQ ID NO: 63 - DNA, 2998 bp, synthetic construct
  source 1..2998: mol_type = other DNA
gctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctt

SEQ ID NO: 64 - DNA, 695 bp, synthetic construct
  source 1..695: mol_type = other DNA
gctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctg

SEQ ID NO: 65 - DNA, 2187 bp, synthetic construct
  source 1..2187: mol_type = other DNA
ccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctt

SEQ ID NO: 66 - DNA, 116 bp, synthetic construct
  source 1..116: mol_type = other DNA
gctagcgtttaaacttaagcttggtaccgagctcggatccactagtccagtgtggtggaattctgcagatatccagcacagtggcggccgctcgagtctagagggcccgtttaaac

SEQ ID NO: 67 - DNA, 6259 bp, synthetic construct
  source 1..6259: mol_type = other DNA
aaatgggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgcgttcaaaatggtatgcgttttgacacatccactatatatccgtgtcgttctgtccactcctgaatcccattccagaaattctctagcgattccagaagtttctcagagtcggaaagttgaccagacattacgaactggcacagatggtcataacctgaaggaagatctgattgcttaactgcttcagttaagaccgacgcgctcgtcgtataacagatgcgatgatgcagaccaatcaacatggcacctgccattgctacctgtacagtcaaggatggtagaaatgttgtcggtccttgcacacgaatattacgccatttgcctgcatattcaaacagctcttctacgataagggcacaaatcgcatcgtggaacgtttgggcttctaccgatttagcagtttgatacactttctctaagtatccacctgaatcataaatcggcaaaatagagaaaaattgaccatgtgtaagcggccaatctgattccacctgagatgcataatctagtagaatctcttcgctatcaaaattcacttccaccttccactcaccggttgtccattcatggctgaactctgcttcctctgttgacatgacacacatcatctcaatatccgaatacggaccatcagtctgacgaccaagagagccataaacaccaatagccttaacatcatccccatatttatccaatattcgttccttaatttcatgaacaatcttcattctttcttctctagtcattattattggtccgttcataacaccccttgtattactgtttatgtaagcagacagttttattgttcatgatgatatatttttatcttgtgcaatgtaacatcagagattttgagacacaacgtggctttccccggcccatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatagccgcggcatcgatgataattcggcttatttaaattccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctcggcgcgcctctcgagttactaactgcagggcacagatgcccattcgctccaagatgagctatagtagcggtcctgggcccgcacgctaatgctggcatttttgcggcagatgaccgtggctgaggtcttgtccgtgaagactctatctttcttttctctcttgctcttgccctggacctgaacgcagaatgtcagggagaagtaggaatgtggagtactccaggtgtcagggtactcccagctgacctccacctgccgagaattctttaatggcttcagctgcaagttcttgggtgggtcaggtttgatgatgtccctgatgaagaagctgctggtgtagttttcatacttgagcttgtgaacggcatccaccatgacctcaatgggcagactctcctcagcagctgggcaggcactgtcctcctggcactccactgagtactcatactccttgttgtcccctctgactctctctgcagagagtgtagcagctccgcacgtcaccccttgggggtcagaagagcctctgctgcttttgacactgaatgtcaaatcagtactgattgtcgtcagccaccagcaggtgaaacgtccagaataattcttggcctcgcatcttagaaaggtcttatttttgggttctttctggtcctttaaaatatcagtggaccaaattccatcttcctttttgtgaagcagcaggagcgaatggcttagaacctcgcctcctttgtgacaggtgtactggccagcatctccaaactctttgacttggatggtcagggttttgccagagcctaagacctcactgctctggtccaaggtccaggtgataccatcttcttcaggggtgtcacaggtgaggaccaccatttctccaggggcatccggataccaatccaattctacgacataaacatctttcttcagttcccatatggccacgaggggagatgccagaaaaaccagggaaaaccaagagatgaccaactgctggtgacacatggtggctcgagtgggcccaagtttaaacgctcctccgacgtccccaggcagaatggcggttccctaaacgagcattgcttatatagacctcccattaggcacgcctaccgcccatttacgtcaatggaacgcccatttgcgtcattgcccctccccattgacgtcaatggggatgtacttggcagccatcgcgggccatttaccgccattgacgtcaatgggagtactgccaatgtaccctggcgtacttccaatagtaatgtacttgccaagttactattaatagatattgatgtactgccaagtgggccatttaccgtcattgacgtcaatagggggcgtgagaacggatatgaatgggcaatgagccatcccattgacgtcaatggtgggtggtcctattgacgtcaatgggcattgagccaggcgggccatttaccgtaattgacgtcaatgggggaggcgccatatacgtcaataggaccgcccatatgacgtcaataggaaagaccatgctaagccgaattatcgcggctatctgaggggactagggtgtgtttaggcgaaaagcggggcttcggttgtacgcggttaggagtcccctcaggatatagtagtttcgcttttgcatagggagggggaaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattccgcattgcagagatattgtatttaagtgcctagctcgatacaataaacgccatttgaccattcaccacattggtgtgcacctccaagcttcgaccaattctcatgtttgacagcttatcatcgcagatccgggcaacgttgttgccattgctgcaggcgcagaactggtaggtatggaagatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcgggcgcgcgtcgaccaccatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaatagacgcgtaaaaagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggtttttt

SEQ ID NO: 68 - DNA, 660 bp, Homo sapiens
  source 1..660: mol_type = genomic DNA
atgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaa

SEQ ID NO: 69 - DNA, 987 bp, Homo sapiens
  source 1..987: mol_type = genomic DNA
atgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttag

SEQ ID NO: 70 - protein, 33 aa, synthetic construct; identical to SEQ ID NO: 32

SEQ ID NO: 71 - protein, 33 aa, synthetic construct
GQSPTLLTSACFFPSAAFTAEFLFSLGSLCQHL

SEQ ID NO: 72 - protein, 33 aa, synthetic construct
RLASAPPQKQKQKLGERLFPLIQAMHPTLAGKI

SEQ ID NO: 73 - protein, 33 aa, synthetic construct
CGHLYAYDWISIPVVYTQVVTVAVYSFFLTCLV

SEQ ID NO: 74 - protein, 33 aa, synthetic construct
DCMSEDGYKLDRIKGEFEFHNVTFHYPSRPEVK

SEQ ID NO: 75 - protein, 33 aa, synthetic construct
MANDSPAKSLVDIDLPSLRDPAGIFELVEVVGN

SEQ ID NO: 76 - protein, 33 aa, synthetic construct
WLGIRISESPEPGQRTFTTFKFCQLPFYKYSEY

SEQ ID NO: 77 - protein, 33 aa, synthetic construct
PPLARPILPRERGAMDRIVEYLVGDGPQNRYAL

SEQ ID NO: 78 - protein, 33 aa, synthetic construct
TCVSLDDGLYECSCAPAYSGKDCQKKDGPCVIN

SEQ ID NO: 79 - protein, 33 aa, synthetic construct
LHVGLCSGPCEMAEQRCCVDYAKRGTAGCKKCK

SEQ ID NO: 80 - protein, 33 aa, synthetic construct
SFESYLKTMPPYFLGPLKKAVRMMGAPNLIADS

SEQ ID NO: 81 - protein, 33 aa, synthetic construct
PKFHSYVSLPFGCTRAVVEYRLLQAAYLAKPGD

SEQ ID NO: 82 - protein, 33 aa, synthetic construct
GEYVDTGKLIDKINLPNFLKVYLNHKPPFGNTM

SEQ ID NO: 83 - protein, 33 aa, synthetic construct
MCTNARRVRKRWLPKINSMLPEGVEMYRTVMGS

SEQ ID NO: 84 - protein, 33 aa, synthetic construct
PDGPLLPSPGLLRLPGGPRHAVSGVPEVYPPGP

SEQ ID NO: 85 - protein, 33 aa, synthetic construct
VGTQATKYNSEVVDKMIEEFLSSFEEKIENLTE

SEQ ID NO: 86 - protein, 33 aa, synthetic construct
TNLPIFKLKESCVRRRYSDFKWLKNELERDSKI

SEQ ID NO: 87 - protein, 33 aa, synthetic construct
LIRWCLALRPSDRPTFEEIQKHPWMQDVLLPQE

SEQ ID NO: 88 - protein, 33 aa, synthetic construct
KFECGWCSGERRCTLHQHCTNPSSPWLDWSSHN

SEQ ID NO: 89 - protein, 33 aa, synthetic construct
HDHTGFLTEYVATRWYSAPEIMLNSKGYTKSID

SEQ ID NO: 90 - protein, 33 aa, synthetic construct
SSEATEVNPESLAKECLEKSLLLLKFLPTGISS

SEQ ID NO: 91 - protein, 33 aa, synthetic construct
VKKDRSASHLDHKLEIRQWRRSFLVSLFFCIPV

SEQ ID NO: 92 - protein, 33 aa, synthetic construct
LFTFAVGVNICLGFTAYRIKRAEGWEEGPPTVL

SEQ ID NO: 93 - protein, 25 aa, synthetic construct
MSQAPGAQLSPPTVYHERQRLELCA

SEQ ID NO: 94 - protein, 25 aa, synthetic construct
MSSNSFPYIEQSGGGEATELGQEAT

SEQ ID NO: 95 - protein, 33 aa, synthetic construct
QPCRKMLPDRSRAARYRANQKLVEYIVKAKGAE

SEQ ID NO: 96 - protein, 33 aa, synthetic construct
PVFHGMLERAPAEPCYRAPMEKLFYLPHVCSYT

SEQ ID NO: 97 - protein, 33 aa, synthetic construct
TYSCVAENILGTGQRGSGAELDVQYPPKKVTTV

SEQ ID NO: 98 - protein, 33 aa, synthetic construct
SPSATEDSLQPATDLLNRSELPQSQKAMQTKDA

SEQ ID NO: 99 - protein, 24 aa, synthetic construct
DLRKVPGTDPAFEVFPLHDHQCVL

SEQ ID NO: 100 - protein, 21 aa, synthetic construct
DALDFKKDKGAFYLVFIWTMT

SEQ ID NO: 101 - protein, 33 aa, synthetic construct
VDNYKQIKADLDQLRLPVEKSELWVEKSSNYEN

SEQ ID NO: 102 - protein, 33 aa, synthetic construct
NAPENGYMKCSSEGDNYGATCEFSCIGGYELQG

SEQ ID NO: 103 - protein, 33 aa, synthetic construct
ESGSSGKSSSSLVRGVHLSSSGPALLAGLVSLD

SEQ ID NO: 104 - protein, 33 aa, synthetic construct
WTEKRASYELEFAKSTTKIAEAGKVSIQQQSHM

SEQ ID NO: 105 - protein, 33 aa, synthetic construct
KILARKKSKRSALENSEEHSTKYSNSNNSAGSG

SEQ ID NO: 106 - protein, 33 aa, synthetic construct
AAAAAALAAALLLLRREDPGLGAGPSMAETEAL

SEQ ID NO: 107 - protein, 33 aa, synthetic construct
LPSFPPFGSMNSNAAGSVSTQANTVQSGQLGGQ

SEQ ID NO: 108 - protein, 33 aa, synthetic construct
SGVVHRLEVDEDFEEHNAAKVHLMVHNLVPPFL

SEQ ID NO: 109 - protein, 33 aa, synthetic construct
LQIKQQELYKNFLRFQVEEKKQREEAERERLRI

SEQ ID NO: 110 - protein, 26 aa, synthetic construct
EQQSLSSDADTLSLTDSSVDPPIQDP

SEQ ID NO: 111 - protein, 33 aa, synthetic construct
DQNTEDYLFQVILEKQIRIPHSLSVKAASVLKS

SEQ ID NO: 112 - protein, 24 aa, synthetic construct
LREDGREQEIRDGGSLRMIHFGLV

SEQ ID NO: 113 - protein, 33 aa, synthetic construct
LLQLWPVQRLTVKKSTDNLFREQKSSLNYLRQK

SEQ ID NO: 114 - protein, 33 aa, synthetic construct
QWLRAVAVESIHRFCMQPQLLRSFCQSYDMKQH

SEQ ID NO: 115 - protein, 33 aa, synthetic construct
LEAIVQLWRIPSFVTELYVNYDCDYYCSNLFEE

SEQ ID NO: 116 - protein, 33 aa, synthetic construct
TPPGQSPGSAPIIINIHRAQLTNPEVKYNYTED

SEQ ID NO: 117 - protein, 33 aa, synthetic construct
PYATVAPSTLAHPQAQVLARQQALQHAQTLAHA

SEQ ID NO: 118 - protein, 33 aa, synthetic construct
QRGLLQQIGDALSSQRGRVPPAAPPAQPRVPVT

SEQ ID NO: 119 - protein, 33 aa, synthetic construct
MFRSKRSGLVRRLWRSGVVPDREEGGSGGGGGG

SEQ ID NO: 120 - protein, 33 aa, synthetic construct
LEIKVSPPEGAETRKVREIGPLCGRRRPRVRKS

SEQ ID NO: 121 - protein, 33 aa, synthetic construct
TPNFIIPAQRAEPMRIVRQPMPPPGDLEPPFQP

SEQ ID NO: 122 - protein, 33 aa, synthetic construct
GTGALGLLLLFLLGYGLRRRPQKAEEPAPQAGG

SEQ ID NO: 123 - protein, 33 aa, synthetic construct
DSHVSEVLQENYRLKNKLEGLISEKNELKMKSE

SEQ ID NO: 124 - protein, 33 aa, synthetic construct
VSAYQVQSPSWMQPQPCILQHPGAVLTPSMEHT

SEQ ID NO: 125 - protein, 33 aa, synthetic construct
GRFSIVKKCIHKATRKYVAVKFVSKKMKKKEQA

SEQ ID NO: 126 - protein, 33 aa, synthetic construct
GILRDPGSEIEDRQYRIDLQSINIGTAQWHQLK

SEQ ID NO: 127 - protein, 33 aa, synthetic construct
DGTQVLKIYVKQTESHYILVSWKVNSNVMTSNL

SEQ ID NO: 128 - protein, 33 aa, synthetic construct
PPGYIPDELHQVARNGLFTSINSEGEFIPESMD

SEQ ID NO: 129 - protein, 33 aa, synthetic construct
PKFRGVKMIPPGIHFLHYSSMDKANPKEVGPRM

SEQ ID NO: 130 - protein, 33 aa, synthetic construct
NVSSAEGWHVNVTLSIRPSTGTGVMLALVSGNN

SEQ ID NO: 131 - protein, 33 aa, synthetic construct
CQRSFRTFQALKKHLEASHLELSEADIQQLYGG

SEQ ID NO: 132 - protein, 33 aa, synthetic construct
DSVDFRLRVSEPKAVFAKEQLACREVQAEVGAS

SEQ ID NO: 133 - protein, 33 aa, synthetic construct
DIKDGRFPYGSTQDYLKPIILIKLVQLGMAKDD

SEQ ID NO: 134 - protein, 33 aa, synthetic construct
ENYRNLAFLGIALSKPDLITFLEQGKEPWNMKQ

SEQ ID NO: 135 - protein, 33 aa, synthetic construct
WNSLGKMSREEAMSAYVTEMKLVAQKVIDTVPL

SEQ ID NO: 136 - protein, 33 aa, synthetic construct
TFQCVVSPSDVAVVWFWDGALLQPSEKFAISQS

SEQ ID NO: 137 - protein, 33 aa, synthetic construct
HVDLFDLESGEYLCPLCKSLYNTVIPIIPLQPQ

SEQ ID NO: 138 - protein, 33 aa, synthetic construct
SEIISKLYIPKRKIISPRSIKDVLPPVEEAVDR

SEQ ID NO: 139 - protein, 33 aa, synthetic construct; identical to SEQ ID NO: 32

SEQ ID NO: 140 - protein, 33 aa, synthetic construct
GDNTGEQVAVKSPKPESGGNHIADLKKEIEILR

SEQ ID NO: 141 - protein, 33 aa, synthetic construct
KDHGVNSFLVYMAFKDLFQLTDCQIYEVLSVIR

SEQ ID NO: 142 - protein, 33 aa, synthetic construct
NTDGKIEFISTMEGYTYPVYGVQWHPEKAPYEW

SEQ ID NO: 143 - protein, 33 aa, synthetic construct
NNSVLQKQQLELMDTVDNLVNLCTKEGVLLKGG

SEQ ID NO: 144 - protein, 33 aa, synthetic construct
DSNREKDMGLFEVFSQHLPTTEPVDSSVSSSIS

SEQ ID NO: 145 - protein, 33 aa, synthetic construct
LKAEVFTEILNYIYSSTVIVKRQETVTDLAAAG

SEQ ID NO: 146 - protein, 33 aa, synthetic construct
HPNNVVSIKYCSHSGLMFSVSTSYIKVWDIRDS

SEQ ID NO: 147 - protein, 33 aa, synthetic construct
TKNKRRKEYFLAVQIRSLEEKIKVKSTEVEILE

SEQ ID NO: 148 - protein, 33 aa, synthetic construct
LVKPQKENEQAEKKNISLAFFLYDLLSLMDRGF

SEQ ID NO: 149 - protein, 33 aa, synthetic construct
VDIVILGLSYANLQTGSGVLSDSLSGGMQLLPD

SEQ ID NO: 150 - protein, 33 aa, synthetic construct
ITKITRRRHENLPHGVASVKEWFNYVTATRNEE

SEQ ID NO: 151 - protein, 33 aa, synthetic construct
AVLKVGWLFPLSEVPNFTLLMDGCGCWRLKEDQ

SEQ ID NO: 152 - protein, 29 aa, synthetic construct
LVAALLLGSVCCGSAQLLFNKTKSVEFTF

SEQ ID NO: 153 - protein, 33 aa, synthetic construct
SHVFGTNTSSLELFLMNGKIKGPCWLEVKSPQL

SEQ ID NO: 154 - protein, 33 aa, synthetic construct
SPYDHTPGMAGSLGYHPYEAPLGSYPYGDPAYR

SEQ ID NO: 155 - protein, 22 aa, synthetic construct
MTGSSLSLAHLLIISGLLCYSA

SEQ ID NO: 156 - protein, 33 aa, synthetic construct
AEEGDELQQRCLDLERHLMLLSEEKQSLAQENA

SEQ ID NO: 157 - protein, 33 aa, synthetic construct
TMYHGVPVVGIPLFGDHYETMTRVQAKGMGILL

SEQ ID NO: 158 - protein, 33 aa, synthetic construct
QPASHILNSEKFPFSKYEDCLEQQMPSLRESPM

SEQ ID NO: 159 - protein, 33 aa, synthetic construct
VEKSPAKKKATKKAVGAGAAKRKATGPPVSELI

SEQ ID NO: 160 - protein, 33 aa, synthetic construct
LLAAGPSAAAAKPNIPKVLLPFTRATRVNFTLE

SEQ ID NO: 161 - protein, 33 aa, synthetic construct
LAPQDMRVFYAHTARAQLPVWNNWTQRAARYEC

SEQ ID NO: 162 - protein, 33 aa, synthetic construct
PAPLRRPLAVLHAPVPTTALARLSRPQRACPSS

SEQ ID NO: 163 - protein, 33 aa, synthetic construct
KHWFVLTDSSLKYYRDSTAEGADELDGEIDLRS

SEQ ID NO: 164 - protein, 33 aa, synthetic construct
VKFLRKPGGNLGKVYHPGSMLSLAPTKGLLNEP

SEQ ID NO: 165 - protein, 33 aa, synthetic construct
DYQVYLNASKVPGFADDPTELSCRVVDTKSGEA

SEQ ID NO: 166 - protein, 33 aa, synthetic construct
QRDDPVLETSHQNFRRSRYQEAASPREALIRLR

SEQ ID NO: 167 - protein, 33 aa, synthetic construct
HLLNYVWPNVFETSPHVIEAVMGALEGLRVAIG

SEQ ID NO: 168 - protein, 21 aa, synthetic construct
SRTSMDDLKAFTSLSLYMKPL

SEQ ID NO: 169 - protein, 33 aa, synthetic construct
CAAYGGYINCMAVLMESNADPNIQDKEGRTALH

SEQ ID NO: 170 - protein, 33 aa, synthetic construct
DNRLPPKKVPGFCSFRDGLSFLVHCCNVIITAQ

SEQ ID NO: 171 - protein, 33 aa, synthetic construct
PYSTAVSTSGCGEHLVHTILARECSHALQAEDA

SEQ ID NO: 172 - protein, 21 aa, synthetic construct
GVLKQYQIKGLEWLVSCTTTT

SEQ ID NO: 173 - protein, 33 aa, synthetic construct
SKSPSKRSKSQDQAKKSKSPTLRRRSQEKIGKA

SEQ ID NO: 174 - protein, 33 aa, synthetic construct
FSLWKGFTPYYACLGPHTVLTFIFLEQMNKAYK

SEQ ID NO: 175 - protein, 33 aa, synthetic construct
DSFLVERGKLLHKRDNDKVDTQEENFLPKYQRV

SEQ ID NO: 176 - protein, 33 aa, synthetic construct
FRTYHQMVLHSRVHRRARREMDSDGDRAARARC

SEQ ID NO: 177 - protein, 33 aa, synthetic construct
LEENINSALDIKVLTQSSQSAVRGGPGRSTMPT

SEQ ID NO: 178 - protein, 33 aa, synthetic construct
GASVNAPPDPCKQSPVRLAAGSGLACFLLWQLQ

SEQ ID NO: 179 - protein, 33 aa, synthetic construct; identical to SEQ ID NO: 35

SEQ ID NO: 180 - protein, 33 aa, synthetic construct
WMGDFVTAWMVTDMMLQAKPYPDWGKSARAFWK

SEQ ID NO: 181 - protein, 33 aa, synthetic construct
PTGRPYNVDTESAQLYQGPHNTLFGYSVVLHSH

SEQ ID NO: 182 - protein, 33 aa, synthetic construct
EKLAAEEQFQALVKQMYQTLQDKTNQIDLLQAE

SEQ ID NO: 183 - protein, 33 aa, synthetic construct
NEKMDSIVQCRALNIAVDLGQIQYLFSDKTGTL

SEQ ID NO: 184 - protein, 33 aa, synthetic construct
EELPVVGQLLRLLLQHTPLRTHMLTNAILVQQI

SEQ ID NO: 185 - protein, 33 aa, synthetic construct
NLPLLQIELLRSAIKALRPGRILVYSTCTLSKA

SEQ ID NO: 186 - protein, 33 aa, synthetic construct
GFQCAAPLLILTNKFCFLSTYVFVSTFLTGKEL

SEQ ID NO: 187 - protein, 33 aa, synthetic construct
VTAAFLVSFMPYHIQRNIHLHFLHNETKPCDSV

SEQ ID NO: 188 - protein, 33 aa, synthetic construct
ARPELLQSHFIPTIGRLRKKAGKVVSEEEQLRL

SEQ ID NO: 189 - protein, 33 aa, synthetic construct
DRVDAQDETAAEFTLRVNLTAARLGQLEGLLQA

SEQ ID NO: 190 - protein, 33 aa, synthetic construct
LVQSRQNLVAEERLSSGILGTIGFGRKSPLSNR

SEQ ID NO: 191 - protein, 18 aa, synthetic construct
MAFRTICVLLEYLFVLSV

SEQ ID NO: 192 - protein, 33 aa, synthetic construct
FEKGRIYTFIGEVVVSVNPYQLLNIYGRDTIEQ

SEQ ID NO: 193 - protein, 22 aa, synthetic construct
WPAVFARVSVFVDWIHKVMRLG

SEQ ID NO: 194 - protein, 33 aa, synthetic construct
VCVSSDTLDGNSSSAGYSCNSPAKVSTTTDSPV

SEQ ID NO: 195 - protein, 33 aa, synthetic construct
VEDAVVFSTELEASAISPRVVVPLAETHCEEQG

SEQ ID NO: 196 - protein, 33 aa, synthetic construct
AAQPYSNLSNLDVLNQVVRERDTKLPKPQLEQP

SEQ ID NO: 197 - protein, 33 aa, synthetic construct
EVKRSQRLRRGPSSPRRPYQEMEYERRGGRGDR

SEQ ID NO: 198 - protein, 33 aa, synthetic construct
ASSLNENVDHSALLKLDTSVSERVSRFDSKPAP

SEQ ID NO: 199 - protein, 21 aa, synthetic construct
CHPGPRDLLLLHLLQRKDNID

SEQ ID NO: 200 - protein, 24 aa, synthetic construct
DLLRSVLQQRLIALGGIIAARISV

SEQ ID NO: 201 - protein, 33 aa, synthetic construct
YTMQKHLEMNPHFGSHQYQILPLHSQIPREEQR

SEQ ID NO: 202 - protein, 33 aa, synthetic construct
CSSSLMALQNAYQAVHSGQCPAAIVGGINVLLK

SEQ ID NO: 203 - protein, 33 aa, synthetic construct
FRLNIQGLERTLLGFVSKTLDSASAQFAASALV

SEQ ID NO: 204 - protein, 33 aa, synthetic construct
INPQGLSDPSPMSDPVLTQDISPPAQGVDHRQV

SEQ ID NO: 205 - protein, 33 aa, synthetic construct
LGPHEEDQVVCGFKKLSEWGLCFHPSTDTHKKL

SEQ ID NO: 206 - protein, 33 aa, synthetic construct
QEKAFFAQLQLGEETGEFLPIQPAQHIQSETSG

SEQ ID NO: 207 - protein, 33 aa, synthetic construct; identical to SEQ ID NO: 36

SEQ ID NO: 208 - protein, 33 aa, synthetic construct
GCCPPGHGSRSRPPAQPACSRAEQDILFLLRSP

SEQ ID NO: 209 - protein, 33 aa, synthetic construct
LVSLQPAQMHRFWRGLSLHLDSVDAVYERTSDH

SEQ ID NO: 210 - protein, 33 aa, synthetic construct
IQLPIIQLRKVWAEAVRYVSGLKEDYSRLFQGQ

SEQ ID NO: 211 - protein, 33 aa, synthetic construct
IFTGQANVVSEYLLAYLFLSLVAFFRLIAFLNL

SEQ ID NO: 212 - protein, 33 aa, synthetic construct
GRRCIRATPRACRARCATCCVRSSAATSPCCGC

SEQ ID NO: 213 - protein, 33 aa, synthetic construct
MAFALAIRDTATYARQLMFSTTLLIVFFTVWVF

SEQ ID NO: 214 - protein, 33 aa, synthetic construct
WRPLLRGPRLSLHTAAKAAATATETTCQDVAAT

SEQ ID NO: 215 - protein, 33 aa, synthetic construct
EALAENPGLVNKSCYEVGWLIKMTLSNPSELDE

SEQ ID NO: 216 - protein, 33 aa, synthetic construct
PSGSFSTPGSATYGRYKPSPERYTAAPHPLLSL

SEQ ID NO: 217 - protein, 33 aa, synthetic construct
LQTRLQPEESTETLDSNYVVGHVLNSRKQKQLL

SEQ ID NO: 218 - protein, 33 aa, synthetic construct
LEVSPFFKCHEVIGSSVLFIHDKKEQAKVWMID

SEQ ID NO: 219 - protein, 33 aa, synthetic construct
VAQPKKKLGRRLVFHSNPPSGNKQQLPLVNETK

SEQ ID NO: 220 - protein, 33 aa, synthetic construct
VHIRRKHSHQYSYLCEVCKYYTVTKGDMERHCA

SEQ ID NO: 221 - protein, 33 aa, synthetic construct
CHVGSEETRARVPVHDLHVGITKRLKTMEVLEG

SEQ ID NO: 222 - protein, 33 aa, synthetic construct
VTDYTFEDCQLALAEGHLRLPADTCLLEFARLV

SEQ ID NO: 223 - protein, 33 aa, synthetic construct
EIHLEIFESASAGTRLLLDPATDPDININSIKD

SEQ ID NO: 224 - protein, 33 aa, synthetic construct
VYVSTQFPKYIQDIVAATLKLPANKVMCHVRRV

SEQ ID NO: 225 - protein, 33 aa, synthetic construct
LTQSKSSPKRGFFREETDRLIKNLLGKRISKLI

SEQ ID NO: 226 - protein, 33 aa, synthetic construct
NNMIHQVPIKSLPQEWLWCEMWCDDASKKRAKT

SEQ ID NO: 227 - protein, 33 aa, synthetic construct
PENIEITVTLFKDPHAEEFEGKEWTFVIENESP

SEQ ID NO: 228 - protein, 33 aa, synthetic construct
AASRPRLDPWKLVSFSRTLTISPPSRPDTPESP

SEQ ID NO: 229 - protein, 33 aa, synthetic construct
SSKTTSVLQWAEKGYYTRSNNLVTLENGKQLTV

SEQ ID NO: 230 - protein, 33 aa, synthetic construct
LDWQPRENPFQNLKVLSVSDQQQNFLELWSEIL

SEQ ID NO: 231 - protein, 33 aa, synthetic construct
ERWECTTYWYIFVFCSAHPAVTEMALFVTVFGL

SEQ ID NO: 232 - protein, 33 aa, synthetic construct
FYKKCAQLTVNLTRFPETVPGELVVPVAGSCVV

SEQ ID NO: 233 - protein, 24 aa, synthetic construct
NNVDLILATPPFSRLEKLYSGEVV

SEQ ID NO: 234 - protein, 33 aa, synthetic construct
KTMKDFVRRRCWARKYKLVTSGPWLEVPPIALR

SEQ ID NO: 235 - protein, 33 aa, synthetic construct
ARRMAFLAKKGYRHDSSTAVVGSPRGHGQSRET

SEQ ID NO: 236 - protein, 33 aa, synthetic construct
WRRAQKLHFLQRGGLKQLSARVRPELKTKQQIL

SEQ ID NO: 237 - protein, 33 aa, synthetic construct
SGRLDQLLSLSMVWANHVFLGCSYNKDLLDKVM

SEQ ID NO: 238 - protein, 33 aa, synthetic construct
ELGSLIRQLSLCSVKLYADPSVPDVVIDILQQI

SEQ ID NO: 239 - protein, 33 aa, synthetic construct
PELSISSLTSPIPENSTETEVALFRIRDRDSGE

SEQ ID NO: 240 - protein, 33 aa, synthetic construct
TSELLGEGAYAKVQGAMSLQNGKEYAVKIIEKQ

SEQ ID NO: 241 - protein, 33 aa, synthetic construct
DWEGNRAYSQYDKFHIGNEKQNYRLYLKGHTGT

SEQ ID NO: 242 - protein, 33 aa, synthetic construct
LRPKLIEELAATRIVDVSTGDSHCLALSHDNEV

SEQ ID NO: 243 - protein, 33 aa, synthetic construct
YAYDKRANPQVGAAFPHIKHNYECLVYVQLPFM

SEQ ID NO: 244 - skipped

SEQ ID NO: 245 - protein, 33 aa, synthetic construct
RRYPSMARIHSMTIEAPIAKDGLRRLSGNEYVF

SEQ ID NO: 246 - protein, 33 aa, synthetic construct
ISTAVRPTTPTTCSRRWWRPSSVGGSASGSRPP

SEQ ID NO: 247 - protein, 33 aa, synthetic construct; identical to SEQ ID NO: 31

SEQ ID NO: 248 - protein, 33 aa, synthetic construct
HLVSRKSISAALEHKVSALLPPAEQTGTWKLKT

SEQ ID NO: 249 - protein, 33 aa, synthetic construct
DLRSRIEVLKRKVIEKLQHIQLLQKNVRAQLVD

SEQ ID NO: 250 - protein, 33 aa, synthetic construct
SKDLLRQLPASNFNSLRFLIVHLKRVVDHAEEN

SEQ ID NO: 251 - protein, 33 aa, synthetic construct
TLLLDGNPFRVPQAAILMKGTAAILEYLRDRIP

SEQ ID NO: 252 - protein, 33 aa, synthetic construct
SILESSSAFPDNAARREVESPPAVCPSDGCTWK

SEQ ID NO: 253 - protein, 33 aa, synthetic construct
GQPYFEAFKKKMPIAFVAKGINKLLNKLFLINE

SEQ ID NO: 254 - protein, 33 aa, synthetic construct
MEDLQDMFIVHTIEEIEGLIAAHDQFKSTLPDA

SEQ ID NO: 255 - protein, 33 aa, synthetic construct
MGGQTALNCGEELFKRGVLKEYGVKVLGTSVES

SEQ ID NO: 256 - protein, 33 aa, synthetic construct
AKGVNGSSQAPTTGKYCRLCDIQFNNLSNFITH

SEQ ID NO: 257 - protein, 33 aa, synthetic construct
HMKLKVQGMECLDGCYVHSSIVPTFHRRCTWLL

SEQ ID NO: 258 - protein, 33 aa, synthetic construct
QASRGEVPSGAYGYSYIPSGAYVYPPPVANGMY

SEQ ID NO: 259 - protein, 33 aa, synthetic construct
YSSLRSIVVANYEESIKMPISEPAPGKKKSQIQ

SEQ ID NO: 260 - protein, 33 aa, synthetic construct
DLQVSAKIGFLKHHENKTYLAVGSMKTLMLNVS

SEQ ID NO: 261 - protein, 33 aa, synthetic construct
ELILPSGARVGHRSLRRYYKQRFGLSRAVAVAK

SEQ ID NO: 262 - protein, 29 aa, synthetic construct
MLGEQGFQSLMETVDTGIVTQLQEFLQGF

SEQ ID NO: 263 - protein, 33 aa, synthetic construct
RQIWWERLQGCQHIVEDWQKILMVRSLVVSPHE

SEQ ID NO: 264 - protein, 33 aa, synthetic construct
STTGSNYYVRILSTIDGELLKPNASVALHKHSN

SEQ ID NO: 265 - protein, 33 aa, synthetic construct
ANELCEVNRKGCPSADPCLPYFCVQGCKLGEAS

SEQ ID NO: 266 - protein, 33 aa, synthetic construct
FEDCTSENTLIKYMTDRILLRESLREADLDHYS

SEQ ID NO: 267 - protein, 33 aa, synthetic construct
WIRPLASGSYPSRKAGATLVVYKDLLVLFGGWT

SEQ ID NO: 268 - protein, 33 aa, synthetic construct
SSDYSTSEMLVNVGNLPLDEFYPAVSMVALMRI

SEQ ID NO: 269 - protein, 33 aa, synthetic construct
CESPTAHCNVLNWEQVHRLDGILSETIPIHGRG

SEQ ID NO: 270 - protein, 33 aa, synthetic construct
NKQNKTPLDKSTSGVSEILLKTQMKMSLKCLAA

SEQ ID NO: 271 - protein, 33 aa, synthetic construct
YVCGGMFFFVSFEGKIVMHKIIYMVLFLFCVAL

SEQ ID NO: 272 - protein, 33 aa, synthetic construct
LPTPGSWWEQLTQASRAYASGGTEGFPLSRWAP

SEQ ID NO: 273 - protein, 33 aa, synthetic construct
YSTLSLFDKYKGKSVDTIRSSVIPRHGLQSLGK

SEQ ID NO: 274 - protein, 20 aa, synthetic construct
MNISLVWCLLVLSFAIRRWW

SEQ ID NO: 275 - protein, 33 aa, synthetic construct
RVELQQEVEKLAHENSSMRLELDALRSKYEALQ

SEQ ID NO: 276 - protein, 33 aa, synthetic construct
SPQREPRGGQLRTPRMWPSCSRSLESLRVGAKP

SEQ ID NO: 277 - protein, 33 aa, synthetic construct
FTLWNESTHYQILLTSFPHVENHSCFEHMHHIP

SEQ ID NO: 278 - protein, 33 aa, synthetic construct
DFVVEAIGTEVGTLGFSIERPSQAKIECDDKGD

SEQ ID NO: 279 - protein, 19 aa, synthetic construct
NVIDSMLFVFGYRAQKNKI

SEQ ID NO: 280 - protein, 33 aa, synthetic construct
MMGSARVAELLLLHGAEPNCTDPATLTRPVHDA

SEQ ID NO: 281 - protein, 33 aa, synthetic construct
PPGLDKRLLPETLGPCYSNSQPVWLCLTPRQPL

SEQ ID NO: 282 - protein, 33 aa, synthetic construct
SFLLSPNNGNLEATSNIEYAHVPHLSPAVIPTR

SEQ ID NO: 283 - protein, 33 aa, synthetic construct
EEDEEDTYYTKDLPIHTCSYCGIHDPACVVYCN

SEQ ID NO: 284 - protein, 33 aa, synthetic construct
LISRRDAEVVLTSRELGSLALNQSTGLPTLTLP

SEQ ID NO: 285 - protein, 33 aa, synthetic construct
DFSSNDSSTCTMGLVLTLWSDTLIHLDGDGGFS

SEQ ID NO: 286 - protein, 33 aa, synthetic construct
SSAAPSVMDLSSRASAALHPGIPSPNCMQSPIR

SEQ ID NO: 287 - protein, 14 aa, synthetic construct
CAVRDKGSARQLTF

SEQ ID NO: 288 - protein, 14 aa, synthetic construct
CSARDLYRNTEAFF

SEQ ID NO: 289 - protein, 14 aa, synthetic construct
CAVSAGNNNARLMF

SEQ ID NO: 290 - protein, 14 aa, synthetic construct
CSARDLYRNTEAFF

SEQ ID NO: 291 - protein, 12 aa, synthetic construct
CALDSSASKIIF

SEQ ID NO: 292 - protein, 12 aa, synthetic construct
CASSRQGQEQYF

SEQ ID NO: 293 - protein, 13 aa, synthetic construct
CAEKGAYGQNFVF

SEQ ID NO: 294 - protein, 18 aa, synthetic construct
CASSSDALAGGIHRKLFF

SEQ ID NO: 295 - protein, 13 aa, synthetic construct
CAFNAGNNRKLIW

SEQ ID NO: 296 - protein, 14 aa, synthetic construct
CASSSFRYNQPQHF

SEQ ID NO: 297 - protein, 11 aa, synthetic construct
CAGVFEARLMF

SEQ ID NO: 298 - protein, 13 aa, synthetic construct
CASSLLQGTTAFF

SEQ ID NO: 299 - protein, 12 aa, synthetic construct
CAVEDQGGKLIF

SEQ ID NO: 300 - protein, 14 aa, synthetic construct
CASSQDFAGSEQYF

SEQ ID NO: 301 - protein, 16 aa, synthetic construct
CAVNDPPVDSSYKLIF

SEQ ID NO: 302 - protein, 11 aa, synthetic construct
CASSFADTQYF

SEQ ID NO: 303 - protein, 16 aa, synthetic construct
CAVNDPPVDSSYKLIF

SEQ ID NO: 304 - protein, 11 aa, synthetic construct
CASSFADTQYF

SEQ ID NO: 305 - protein, 12 aa, synthetic construct
CALAGGSYIPTF

SEQ ID NO: 306 - protein, 11 aa, synthetic construct
CASADSYEQYF

SEQ ID NO: 307 - protein, 12 aa, synthetic construct
CAFGDSWGKFQF

SEQ ID NO: 308 - protein, 15 aa, synthetic construct
CASSIDQTGITEAFF

SEQ ID NO: 309 - protein, 14 aa, synthetic construct
CAAIRSGSARQLTF

SEQ ID NO: 310 - protein, 16 aa, synthetic construct
CASSQDLGVAYNEQFF

SEQ ID NO: 311 - protein, 12 aa, synthetic construct
CAYSGSARQLTF

SEQ ID NO: 312 - protein, 14 aa, synthetic construct
CASSFGGNTETQYF

SEQ ID NO: 313 - protein, 14 aa, synthetic construct
CGTEAIGGTYKYIF

SEQ ID NO: 314 - protein, 14 aa, synthetic construct
CASSFGGNTETQYF

SEQ ID NO: 315 - protein, 13 aa, synthetic construct
CIDSGGSNYKLTF

SEQ ID NO: 316 - protein, 12 aa, synthetic construct
CASSPAGPAQYF

SEQ ID NO: 317 - protein, 15 aa, synthetic construct
CAGRLSSGGSYIPTF

SEQ ID NO: 318 - protein, 15 aa, synthetic construct
CASSAIGTPSGEQFF

SEQ ID NO: 319 - protein, 14 aa, synthetic construct
CAVGSDGAGNMLTF

SEQ ID NO: 320 - protein, 15 aa, synthetic construct
CASTRDESYNSPLHF

SEQ ID NO: 321 - protein, 19 aa, synthetic construct
CAVGGRSNAGGTSYGKLTF

SEQ ID NO: 322 - protein, 15 aa, synthetic construct
CASTRDESYNSPLHF

SEQ ID NO: 323 - protein, 15 aa, synthetic construct
CAASVGGAGANNLFF

SEQ ID NO: 324 - protein, 13 aa, synthetic construct
CASSVDIYNEQFF

SEQ ID NO: 325 - protein, 14 aa, synthetic construct
CAMREPYNFNKFYF

SEQ ID NO: 326 - protein, 14 aa, synthetic construct
CATHEGWDTGELFF

SEQ ID NO: 327 - protein, 13 aa, synthetic construct
CAAGGTSYGKLTF

SEQ ID NO: 328 - protein, 14 aa, synthetic construct
CATTSGSPAGELFF

SEQ ID NO: 329 - protein, 14 aa, synthetic construct
CAAETRNNNARLMF

SEQ ID NO: 330 - protein, 14 aa, synthetic construct
CAGRLAGASGELFF

SEQ ID NO: 331 - protein, 13 aa, synthetic construct
CAFLLSDGQKLLF

SEQ ID NO: 332 - protein, 14 aa, synthetic construct
CAGRLAGASGELFF

SEQ ID NO: 333 - protein, 14 aa, synthetic construct
CAAAGYSSASKIIF

SEQ ID NO: 334 - protein, 15 aa, synthetic construct
CASSLGPPGQTEAFF

SEQ ID NO: 335 - protein, 15 aa, synthetic construct
CAMSAPWDTGNQFYF

SEQ ID NO: 336 - protein, 15 aa, synthetic construct
CASSHGQGKDGELFF

SEQ ID NO: 337 - protein, 12 aa, synthetic construct
CAAEGNTDKLIF

SEQ ID NO: 338 - protein, 16 aa, synthetic construct
CASSQDPAGRWTEAFF

SEQ ID NO: 339 - protein, 11 aa, synthetic construct
CTTSGTYKYIF

SEQ ID NO: 340 - protein, 14 aa, synthetic construct
CASSPRQGPGELFF

SEQ ID NO: 341 - protein, 15 aa, synthetic construct
CAVEEISGGYQKVTF

SEQ ID NO: 342 - protein, 15 aa, synthetic construct
CASSENRGRADTQYF

SEQ ID NO: 343 - protein, 15 aa, synthetic construct
CAVEEISGGYQKVTF

SEQ ID NO: 344 - protein, 15 aa, synthetic construct
CASSENRGRADTQYF

SEQ ID NO: 345 - protein, 15 aa, synthetic construct
CAVEEISGGYQKVTF

SEQ ID NO: 346 - protein, 15 aa, synthetic construct
CASSENRGRADTQYF

SEQ ID NO: 347 - protein, 12 aa, synthetic construct
CAESIGDYKLSF

SEQ ID NO: 348 - protein, 17 aa, synthetic construct
CASSQELVGPNTGELFF

SEQ ID NO: 349 - protein, 14 aa, synthetic construct
CAATSMDSNYQLIW

SEQ ID NO: 350 - protein, 13 aa, synthetic construct
CASSIEQGVAQYF

SEQ ID NO: 351 - protein, 15 aa, synthetic construct
CAFTYSGGGADGLTF

SEQ ID NO: 352 - protein, 16 aa, synthetic construct
CATSREGATFYNEQFF

SEQ ID NO: 353 - protein, 20 aa, synthetic construct
CIVRVAGFLYSGGGADGLTF

SEQ ID NO: 354 - protein, 16 aa, synthetic construct
CASSVGEFTGTDTQYF

SEQ ID NO: 355 - protein, 12 aa, synthetic construct
CVVSRGDYKLSF

SEQ ID NO: 356 - protein, 13 aa, synthetic construct
CAINPDSNQPQHF

SEQ ID NO: 357 - protein, 10 aa, synthetic construct
CAVGNARLMF

SEQ ID NO: 358 - protein, 13 aa, synthetic construct
CAINPDSNQPQHF

SEQ ID NO: 359 - protein, 14 aa, synthetic construct
CAVSPMTGFQKLVF

SEQ ID NO: 360 - protein, 17 aa, synthetic construct
CASSELEGDGSGNTIYF

SEQ ID NO: 361 - protein, 13 aa, synthetic construct
CAASGTSYGKLTF

SEQ ID NO: 362 - protein, 17 aa, synthetic construct
CASSQGGSGTLFHEQFF

SEQ ID NO: 363 - protein, 13 aa, synthetic construct
CAVPHGATNKLIF

SEQ ID NO: 364 - protein, 12 aa, synthetic construct
CASKGVVYEQYF

SEQ ID NO: 365 - protein, 15 aa, synthetic construct
CAEIPFSGGYNKLIF

SEQ ID NO: 366 - protein, 15 aa, synthetic construct
CASSGRTGGGYEQYF

SEQ ID NO: 367 - protein, 6 aa, synthetic construct
HHHHHH

SEQ ID NO: 368 - DNA, 66 bp, Porcine teschovirus
  source 1..66: mol_type = genomic DNA
ggaagcggagctactaacttcagcctgctgaagcaggctggagacgtggaggagaaccctggacct

SEQ ID NO: 369 - protein, 22 aa, Porcine teschovirus
GSGATNFSLLKQAGDVEENPGP

SEQ ID NO: 370 - DNA, 63 bp, Thosea asigna virus
  source 1..63: mol_type = genomic DNA
ggaagcggagagggcagaggaagtctgctaacatgcggtgacgtcgaggagaatcctggacct

SEQ ID NO: 371 - protein, 21 aa, Thosea asigna virus
GSGEGRGSLLTCGDVEENPGP

SEQ ID NO: 372 - DNA, 69 bp, Equine rhinitis A virus
  source 1..69: mol_type = genomic DNA
ggaagcggacagtgtactaattatgctctcttgaaattggctggagatgttgagagcaaccctggacct

SEQ ID NO: 373 - protein, 23 aa, Equine rhinitis A virus
GSGQCTNYALLKLAGDVESNPGP

SEQ ID NO: 374 - DNA, 75 bp, unidentified
  source 1..75: mol_type = unassigned DNA
ggaagcggagtgaaacagactttgaattttgaccttctcaagttggcgggagacgtggagtccaaccctggacct

SEQ ID NO: 375 - protein, 25 aa, unidentified
GSGVKQTLNFDLLKLAGDVESNPGP

SEQ ID NO: 376 - DNA, 99 bp, synthetic construct
  source 1..99: mol_type = other DNA
gccgtgagccactggcaacagcagtcctatctggactccggcatccactatggcgcaacaacaaccgccccatctctgtctggcaagggaaatcctgag

SEQ ID NO: 377 - DNA, 99 bp, synthetic construct
  source 1..99: mol_type = other DNA
ggaggccctcaggccctggtgaatatcatgagaacctatacatacgagatcctgctgtggacaacctccagggtgctgaaggtcctgagcgtgtgcagc

SEQ ID NO: 378 - DNA, 99 bp, synthetic construct
  source 1..99: mol_type = other DNA
ccagatagaaaggccgcagtgagccactggcagcagcagtcctacctggactccggctcccacagcggcgccaccacaaccgccccaagcctgagcgga

SEQ ID NO: 379 - RNA, 99 nt, synthetic construct
  source 1..99: mol_type = other RNA
gccgtgagccactggcaacagcagtcctatctggactccggcatccactatggcgcaacaacaaccgccccatctctgtctggcaagggaaatcctgag

SEQ ID NO: 380 - RNA, 99 nt, synthetic construct
  source 1..99: mol_type = other RNA
ggaggccctcaggccctggtgaatatcatgagaacctatacatacgagatcctgctgtggacaacctccagggtgctgaaggtcctgagcgtgtgcagc

SEQ ID NO: 381 - RNA, 99 nt, synthetic construct
  source 1..99: mol_type = other RNA
ccagatagaaaggccgcagtgagccactggcagcagcagtcctacctggactccggctcccacagcggcgccaccacaaccgccccaagcctgagcgga

SEQ ID NO: 382 - protein, 33 aa, synthetic construct
AVSHWQQQSYLDSGIHYGATTTAPSLSGKGNPE

SEQ ID NO: 383 - protein, 33 aa, synthetic construct
GGPQALVNIMRTYTYEILLWTTSRVLKVLSVCS

SEQ ID NO: 384 - protein, 33 aa, synthetic construct
PDRKAAVSHWQQQSYLDSGSHSGATTTAPSLSG

SEQ ID NO: 385 - DNA, 4785 bp, synthetic construct
  source 1..4785: mol_type = other DNA
atggattggacctggattctctttctcgttgcagctgctacacgcgttcatagcgccgtgagccactggcaacagcagtcctatctggactccggcatccactatggcgcaacaacaaccgccccatctctgtctggcaagggaaatcctgagcggggcagaaagcgcaggagccctgaggaggaggccagactgaagatcctggaaaccctgaagctgctgctgcagcacacatggccaagagtgtcctgcagactggtggtgctgctgaagagaggccgcaaacggaggtccctgttcctgcacctgatctccaacaaggtgtgcctgcggcacctgctgcacaagcgcaaggagacatggtgccctagaccaccacacccccaggccctgaggggcaggaaaaggaggtctatcaccatccagaacgcatacgagaagcagcacccttggctgtacctgagctccctgggcttctgctatctgatctatttcaattccatgagccagatgagaggcaggaaacggagatccctgtacctgagacagctgccagagcccctgatcagcttcagactgtacagagagctggtgggactggccaaggacagcctgaaggccgaggccgaggcacgcggcagaaaacggagatccaaggagctgtttggcaaggggcctcagaatgcctttttcctggtgaagctgtgggccgatctgaattgtaatatccaggacgacgccggagccttctatagaggccggaagcgcaggtccgagctgcagtccgtgatctcccacctgctgagggtgagggccagagtggccgataggctgtgtggcgtgtacaaggtgcacggcaattacggcagagtgagggggaggaagagaagaagcatcctgagcgaaaccacctgcggctgcttcaccttccgcccccagaccaagatctctacccagatctgcgccgcaaaggcctggaggaaggcctgctccaggggacggaaaagaaggagctggccaagacccctgccactgcgccctccaagaaggccacctctgtggaaaacctctgccccaagacccgcacctccaagagacgcatgcccatgtctgaggggacggaagcgcaggtcttatcagaccagaggctgtagccagccagagagagtgaagtttacactgagcatgctggtgacaatggcacccatcgtgctgatcctgctgggcctgctgagaggccggaagagacgcagctttcacggctatgaggccgtgaaggaggccctgatcgacaacggcgagagagtgttctggaagaggcagtttcctaacatctctaagaactacaggggacgcaagagacgctccacactgctgcagaagctgctgtccaagaagcctgaggaccgccccaacgcctccgagatcctgcgcacactgacagtgtggaagaagtctccagagaagagaggcaggaagaggagatccagcctgacctccctgcacgccgtggacggagactccaagctgttcaggacaagacggccacggtccagctccgacgcactgagcgcctcctttaatggccggggcagaaagcggcggtctacagtgaggggagataccaccaataccgtgctgcagggactgaacgagggcacacagtatgcactgtctgtgacagcactgtatgcctccggcgcaggcaggggcagaaagcggagatccaagcacctgtccggagagtttggcaagccttacttcatcaagctgatggtgttcgtggccgaggagagaaagcactacaccgtgtaccccccaccacacagaggccgcaagcggcggagcccactggccagctgtgccggactgagccctacctctgagcagaccaccctgcctgccacagtgaatctgaccgtgagctctagctcttatggatctcagaggggaaggaagcggagaagccaggaggtggataggcagcgggccctgcagcagaggctggagatggagcagcacggcatggtgggcagcgagatctcctccagccggacatctgtgtctcgcggcagaaagcggcgctccaggcagtctgagggccacagcgtgatggacacactggcagtggccctgctggtggccgaggaggccatcgaggaggccatctctaaggcagaggcctataggggaaggaagagacggagcctgtttgaggagaagcactactacaaggtggattacagcagattccacaacacatatgaggtgcctaataccccactgtgctctgcccgcgatctggccagaggcaggaagcgccggagcacacctgtgacccccgagatggagcgggcaaagaggaagcaggagaatatcagctccgtgctgtactccgattcctttagaaagcagatccaggggaagaggggcagaaaaagaaggtctatcgtggaccccgccgtgatgggcggctttgccaagtatgagaaggcctactttaccgaggagtatgtgagggatcacccagaggaccaggataagctgcgcggcagaaagaggaggagcatgatcctgacaaatatcctgtccagcccctactttaaggtgcagctgtgtgagctgaaaacctaccacgaggtggtggatgagatctactttaaggtgcgcggcaggaagagaaggagctggagcctgagcggagacagcaggctgaagatgagaaccgagctgggagagtatatcagacggatcacaaccgtgcctctgcccaccgcaccaaacatcagaggccgcaagagacgcagctccaagtatttccccagctcctctcccgtgaacatcaggctgagctctcagtccgtgagcgacacctttgtgaaggaggtgctgaagtggaagtatgagagaggccggaaacgcaggagctacctgctgtctgatctgggacctcaggagaggatgcacatctctaagtttaccggcctgagcatgaccacaatctcccactggctggcaaacgtgaagcggggcagaaagaggcgctctaagcggtgctcttgctccagcctgatggacaaggagtgcgtgtacttctctcacctggacatcatctgggtgaacacacccgagcacgtggtgccctacaggggccgcaagcgccggtctaaagcaaatacctacgagaagtattggcccttttaccagaagcagggagtgcactacttcccaaaggatcacctgaagaaggcagtggccgagatcgagcgcggacggaagaggcgcagcggactgccatcctttcgggatcagcagtgtcaggcccacgacaggctgatccctaagaagaagggcctgctgacagccgtggtggtggacgacaagcctagaggacgcaaaagaagaagcccacacctgtacagggatggccacgaggaggccctgagcaagtttacatctaagaagctgctgctgctggtgtgttttctggattacgccaagatctctagaggacgcaagcgcagaagcctgatgccagacaagcccctgtccgccaatgtgcccaacgataagttcacacagaatcccatcccaaggggccgcaagcggaggtcctattcccagtacagccacctggtgggccaccgcagggtgcacacaggcgagaagccttacaggtgccacgagtgtggcaaggcattcaaccagggatctaggggccgcaaaaggaggagctctgatcagcagaccgtgcctggctccgagcaggtgcacgaggatctgctgatctctccacagtcctctagcatcggctccgtggacgagggagtgagcagggggcgcaagaggcgctcttttccaaattgctgtaacacaagcaccgtggccgatgcctacaggtggtgcaatcacaccgtgggcaacagaacagtggtcgaggagggctattactatcggggcagaaagagaaggagcaaggccacccagagacgccctcacggacgcgagaggcgggtggcatctatggtgagctataaggaggagagcggctccgacgaggcaggctctggctccaggggccgcaagagaaggtccgccgacaacttccagtcccagctggccgccaagaagggccagtatgacggccaggaggcccacaacgatgccatgatcctgtacgataagtacttctccagaggaaggaagaggcgctctttcttcggacacaatgcagagaaggagaaggcccagctggccgcagaggcccagaagcaggccaacagggtgtccggctcccgcgagccaaggcccgcaagaggcaggaagagacggtccatggtgttcggagagtttttccaccggcccggccaggacggcgagctggtgaacctgaacgtggggggcttcaagcagtctgtggatcagcggggcaggaagagaagatctggcaaggtgttcagctattatagctgcctggccagacacagaaagatccacaccggggagatcccttacaagtgtaacgactgtggcaaagcctacacacgcggccggaagagaaggtctgacgaggaggcaaagaccaccaccaccaacacccagatggagggagatgatgaggcagcatttctggagagactggcacggagagaggagcggcggcagagaggcaggaagcggaggtctagccgctatctgatcagatctgatgacatgatcgagacagtgtataacaatcgcggcgagatcgtgaaaaccaaggagagacgggtgttcatgctgaat

SEQ ID NO: 386 - DNA, 4785 bp, synthetic construct
  source 1..4785: mol_type = other DNA
atggattggacatggattctctttctcgttgcagcagctacacgcgttcatagccccatgctgaccagaagaatcggagtgggcgtggccgtgctgaataggcacctgtatgccgtgggcggctttgatggaaccaacagactgaactccgcccgcggccggaagaggcggtctggaggccctcaggccctggtgaatatcatgagaacctatacatacgagatcctgctgtggacaacctccagggtgctgaaggtcctgagcgtgtgcagccggggacggaagaggaggagccagcaggagatcagatccatccacgtgaaacgccacctggagcctctgcctgccggctatctgtataacggcacccagtttgtgaatttcttcggcgacagaggcagaaaacgcaggtccatcgcaaccatggtgagcggctcctccggcctggccgccgccaggctgctgtctcacagcttcctgctgcctcagaatggcatccgccactgtagctacagaggcaggaaacgccgctcctatcggcggaatatctggaatgacgccaagaccgtgtccgtgatcacaaccgcctgttttagcaaggtgaccaagatcctggtggccgcactgacattccggggacgcaagcgcagatccgagaagagggaggccgtgaacagcctgcaggaggagctgagcaatgtgcaggaccacctgaatctggccaagcaggacctgctgcacaccacaaagcacagaggccgcaagcggagatccgcctcctggtacagcctgtccaagggaatgcaggatttcaactacctgcctacaaattgctttgagatcacactggagctgtcctgtgacaagtttcctaggggccggaagcgccgcagccaggagctggagagcgagcccaggtcctggtccctgctggagcagctgagcctggccggcgccgacctggccgcacccggcgtgcagcagcagctggagcggggcaggaagaggagaagcatgcagccactggccctgagaaccgggctggtgatggccgcactgagcgcctgcgcaatgccctcctctacccacgcctcccaggtgccttccccaacaaggggcaggaagagaaggagccacgatcaccctgtgctgtctgtggactgctatctgaatctgggcagccccatctctgtgtgctacgtgagctccaggcctcactctctgaatatcagccgcggaaggaagagaaggtccgattcctggaagaaccagacctttcagaatgagtctaggacaagcagcagcttccccagcgtgtataccatcacatctaacgacatcagcgtgaataccagaggcaggaagaggcgctccgcagtgagagtcgtgggcgatggcgcctctgtggatctgctgctgctgtcttgtacatggcggatgagcgccgcagtggtgggaggcccatggaggaccagaggacggaagcggaggtcttacgtgtttatctatcaggccctgctggagcactacctgtatggagataccgagctggagatgacctctctggagacccacctgcagaagatctataacagaggcagaaagcggagatctgagcagaccgcagagaagcccgtgcccgccctgtccagcaccgcctggattccaaaggaggccgtgacccccctggacgagcaggagggcgatggctccagaggcagaaaaaggcggtccgatgtgaacgtgagaggcgagcggggcaagacccccctgatcctggccatggagaaaaagcacctgggcctggtgcagcgcctgctggagcaggagcacaggggaaggaagaggcggtccgaggtgctgtatgattccctgcagctggcccacaagggcatcctgaattctttctacggctacgtgatgagaaagggcgccaggtggtattctatggagcgcggacggaagagaagatctacctgggacccccctgtccccaagtgtctgaaggtgctgcccccatccaataccaagcctccagccctgagccacagcgtgtctacaagctccaccacaaggggcagaaagagaagatccgagctgtccctggaggcactggcacccgggggactgcagcagatcctgtctttcatctatacatctaagctgctggtgaacgccgcaaacgtgcacgagaggggcaggaagcggaggagcctgaacctggaccagctgaaggaggagatcgcctttctgaagcacaccgagtacaaggagaataacatctctaccatcatgccattcagagggagaaagaggaggtctggcgacacatctacaagctccctgctgtccgagatgagcagcgtgttcagacgcctgctgcccccaagcctggatacctatagcgagtgctccgaggtgagggggcggaagaggagatcccaggaaaccatcgacaaactgagaggcatcgtgagcgagaaaacaaacgagatcagcaacatccagaaggatctggagcacagcaatgacgccctgaagagaggcagaaagagaaggagccccctggccggcttcgggtatggcctgccaatctctaggctgtacgccaagtactttaagggcgatctgaacctgtacagcctgtccgggtatggcaccagaggaaggaagaggagatctcagtctccagagagcctgctgcagctgaaggccctgaagccaggcgtgatccacatcctgggagtgaaaacatctagatttctgtgccagcgcccagacagaggaaggaagcggagaagcggatgcgagctgatctctacaccagatcaggaccacaccgattttacagagtgtctgaagatgctgcagaagaagatcgaggagaaggatctgaaggtgcgcggcaggaagcgccggagcatgggagtgctgggcatcctgtgctttctgatcttcctgggcaagacctggggccaggagcaacggggcagaaagcgccggtcctgtgaccactattggccagccgaccaggatagcctgtactacggcgatcacatcctgcagatgctgtctgagtccgtgctgcctgagtggacaatcagacggggcagaaaaaggagaagcacaagacacaagagactgcacaccggggagaaaacctacaagtgtggagagtgtggcaaagcattcaaggagtctagcgccctgacaaagcacaagatccgcggcagaaagcgccgcagcaacatcgaggaggagaggtactccgagagagtgtacgcaatcagagagggcgagacactggagctgacatgcctggtgaccggacacccacgcccccagcgcggcaggaagagacgctcctataaccacctggtgtactctcagatccccgcagcagtgaagctgacataccaggacgcaatcaacctgcagaactacgtggaggagaagctgatcccacgcggacggaagaggagatcttccgattctgaggaggtgtctttcatcgtgcagctggtgcggaagctgatgatcatcatctcccgccctgcaagactgctggagtgcctggagttcgaccggggcaggaaacgcaggtctgtgaacagcaagagcggacctgagggcgattccaagatgtcttttagcagaacccactccaccgcaaccatgcctccaccaatcaatcccatcctggcaagaggcagaaagaggagatctgtgatcgcctcctataaccagctgtctagctttcggcagccaaaggtgatgaggtacagagtggtgaacctgtacgaggacgatcacaagaatccaacccggggcagaaagaggaggagctcctctcagacactgcctccagattccacagcaaccgacatcaccagactgcagccaggaacaacatatcaggtggccgtgagcgtgctgaggggaagaagaggcagaaagcgcaggtctgtgggcgacatcgtgatcatcaagggaaaggagtacatccctgcagacacaatcctgctgtccagctccgagcctcaggccatgtgctatatcgagacacgcggcaggaagcggcggagccaggagaccaagaaggtgctgcagtccgagggcgaggcagtggacccacaggccaatgtgatcaagcagaactccggagccgccacctctgaggaaaccaggggcagaaagcggcgctccttcacagacgccgacgacgtggccatcctgacctacgagaaggagaacgcccgcagccccagctccgtgacaggcaatgcactgtggaaggccatggagaggggccgcaagaggcgcagcatcctgagggacaccggccctcggaggaggggacgccggcctcgctctaagctgctgaaggccccaagcatcgtggccgatagcccatctggaatgggccggggccgcaagaggaggtccgagtggttctgtcggatcaacgatatctctcagcgcggatttctggtgggcatcctgaagcagctgaattctctgtacctgctgcactacttccagaacaggggacggaagaggcgctctctgtgcaagggcgccgcaccacctgattctcctgtggtgtacagcgactccgccggctacaataagcagtggcaccccacctgcttcgtgtgcgcaaagagaggccggaagagaagaagccagctgtttccaatgtggagattccccttctatctgtccgccgtgctgtgcgtgttcctgtacttttactgtgtgatcagggatgtgatctatccatat

SEQ ID NO: 387 - DNA, 4821 bp, synthetic construct
  source 1..4821: mol_type = other DNA
atggattggacttggattctctttctcgttgctgctgctactcgcgttcacagtgacctgatcaagagcatgctgcggaatgagaggcagttcaaggaggagatgctggccgagcagctgaaacaggcagaggagctgaggcagtacaaggtgagaggcagaaagcgcagatccccagatagaaaggccgcagtgagccactggcagcagcagtcctacctggactccggctcccacagcggcgccaccacaaccgccccaagcctgagcggaagaggccggaagcgcagatctcggctgagaaagatcgccgacgagaatggcgcatatctgctggccgacatggcccacatctccggactggtggcagccggcgtggtgccttccccatttcgcgggaggaagaggcgctccgacggaatcgagcggacatgtgataccctgctgatgtgcatcgtcacaatgctgaatcagggcctgagaaacggcggaggagtgggcgacgtgctgcggaggggaaggaagcgcaggtctgcccctaaggccgagcccgcaatcatcccagcaaccctgaacgagccaattggactgaaggcctctgactttctgccagccgtgaagatccctcagcagagaggccgcaagaggagatcctgcgtgccccacaggtacgcccaggaggtgtctcggctgtgcctgctgaccgcaggcacctacaaggtggtgccctctacctatctgcctgataccgagcggggaaggaagagaagatcttctaagagcgagagccccaaggagccagagcagctgaggaagctgtttctgggcggactgtccttcgaaaccacagatgagtctctgagatctcacttccggggcagaaaaaggaggtctagcctgccaatgcagtggctgcacagacctctgctggagggcaggctgtctctgaattacgattgttttaagaccgatttcaaggagcaggatatcaagcgcggccgcaagaggcgctctgatgactatggcagggacctgtcctctgtgcagacagtgctgacaaagcaggagacatttgatgcaggcctgcaggcctttcagcaggagggcatcgccagagggaggaagcggagatccgagatgctgagattcggcgacagaatgttctacaaggattggtggaattacacctcctattccaattactacaggacctggaatgtggtggtgcacgaccggggcagaaagagacgcagctccgtgaagaaggagaaggtggtgacaatcaccctggataaggagatggccttcagcgtgctgctgcacagagtgtggaagaagcaccctgtgaatgtgcgcggcaggaagagaagaagctccgatggcgtgggagcccctccccacagcgccgccgccgccctgcccccaccttccctgcggctgcgggccaccctgcgcccagagcctgtgtgcagacgcggcagaaagcggaggagcgtgaacggcgatcgcagatggatcacccagctgcactttgcctttcaggaggagaactatctgtatctggtgatggagtactacgtgggcggcgatctgaggggccgcaagcgccgctctaagctgcactttatcatcggcaacggaatcctgaggcctgccctgagatccgtgcctgagtctctgctggtggccgagtggtgcctgtgccagccttctcggggccgcaagcggaggagcttcgtgaccctggaggagggcaagggcgacctggaggagtatggccagtatctgctgcacacagtctttaaaaacggaaaggtgacaaagtcctactcccgcgggcggaagcggagatctagcggcgccttcctgctgctgtttctgctggtgctgaccgtgaccgagcccctgcgcccaaagctgaggtgcaatccaggccagtttgcctgtaggagcagaggacgcaagcggagatctggcaagctgcaggagacccaggacagggccaagaggcaggaggacctgctgatgaaagtggagaatctggcactgaagaataggttccaggcagtgctgagaggcaggaaaagaaggagcctggaggagatcctgccactgggaccaaataaggccctgttttataagaggatggcatcctatctgctgcctggccagaagtccccctggttcttccagagaggccggaagaggcggagcaggggctctctgaatccagagaaccccgtgcaggtgagcatcaaccagttcaccgccgcaatctacgatctgctgagactgcacgccaattctggcaggagaggccggaagcggaggtcttttgataccaacggagatggaaccatcgacttccgggagtttatcatcgtgctgtctgtgacatccagaggaaagctggagcagaagctgaagtgggccaggggcagaaagaggaggagccagctgctggtgcagcaggtggccctgggcaccatgatgcactgccagacactgaaggaccagccttatgcctacagcaaggagaactgggagaaaacaagaggccgcaagcgcagatccggaggcaactctctgctgggcaagaagtgtttcgccctgcgcatggcaagacgcctggccaaggaggagggctggctggcagagcacatgctgatcctgagaggcaggaagaggcggagccaggtgacatctagagatggcagactgctgagcacagtgatcagggcactggatacaagatccgacggcccattctgtaggatctgccacgagggggcacgcggcagaaagagacggtccaaggagtgtggcaagaccttcagctctagacggaacctgaggagacacatcgtggtgcagggcggcaataggccatataagttcccaatcgaggacatgagaggcagaaagagaaggtccgaggaggccaagaagaagctgctgaaggacgccgaggcactgagccagagcctggaggagaaggcactggcctacgataagctggagaaaaccaaaaatcggggcaggaagaggagatctcacgaggggcgcctgagctacatgagatctaaaaaggtgagcaactgtaaccagtataggctggggaagaagaagggcctgcctagcagaatggtggtgcgcggcagaaagcgcaggtccaagcacagatgcgagtatctgcactctaagctggcacacatcaagcgcctgctggcagagtatgaccagagacagctgcaggcatggcctcgcggccgcaagcggagatccctgaactggatgatcagaatgatgtccgatctggagtccaacatcgtggccgtggagtccgtgaaggagtactctaagaccgagaccgaggcaccctggaggggcaggaagcggagaagcgtgatcacagccggctccgacgccagctccaagctgagggccctgcagtaccggtgcctggtgcctggctggtatgcaacacacatcgagcgctggctgaggggccggaaaagaaggtctgacctgaagcagcaactgctgctgaataagctgatgggcaaggatgagtccacctcccggcatcggcgcagcctgagcccaatcctgcctggaaggcaccgcggcaggaagcgcagaagcatcgtggcaatcgagaacatcctgagctttatgtcctacgacgagatcagccagctgaggccagtgtgtaagcggatggacctggtgtgccagagaatgcggggaaggaaaaggcggagccccaggtacagattcaggaagagagataaggtgctgttttgcggcaggaagatcatgcggaaggtgtcccagtccacctcctccctggtggatacaagcaggggcagaaagcggagaagccagatcacagccgtgatcgtgaaccgcaaagagcacaacctgaagaagggcgcaggataccacctggacctgttctgggtggccatcctgatggtgatccgcggcaggaaacggagaagccacctgagagtgcacagcagggagaagccattcgactgcgaggagtgttggaggtccttcaaggtgaacacccacctgtttagacaccagaagctgcacagaggccggaagagacggtcccacgcaaatcacgccctgcctgtgtatgtgttcacaaggcccacatactctaggaggctgtctggcctgtccgagatggatctgatctctacaatcggaagaggacggaaaagaaggtccccctcccgcggaggccacatggacgacggcggatacagcatgaactttaagatgagctcctccagaggccctctgccagtgaagagagggccacctcgcggccggaagaggaggtctgccgccgcaccaagcggaccagcccccaccaccagcatgcgcttcacactgcccccaggaacctctacaaacggcaaggtgctggccgcaaccgcccccaggggccggaagagaaggagcacactgcgcgtggtgctggccaccctgggagtgggagccgcaagcctgggcatcacctgttataccgcccaggagaacgagctgatcccctctatcatccgcggacgcaagcgccgcagcatgtctgccccccctgtgctgaggccccccagccccctgctgccaggcgcagccgccgcagccgcagccgccgccgcactggtgcccggatctggccccagaggcagaaagaggcggagcaagtacaatggacctccattccctccagtggccagcacatttcagccagccgccgacatcctggataaggtgatcgagaggaaggaaaccctggagaat

SEQ ID NO: 388 - protein, 1595 aa, synthetic construct
MDWTWILFLVAAATRVHSAVSHWQQQSYLDSGIHYGATTTAPSLSGKGNPERGRKRRSPEEEARLKILETLKLLLQHTWPRVSCRLVVLLKRGRKRRSLFLHLISNKVCLRHLLHKRKETWCPRPPHPQALRGRKRRSITIQNAYEKQHPWLYLSSLGFCYLIYFNSMSQMRGRKRRSLYLRQLPEPLISFRLYRELVGLAKDSLKAEAEARGRKRRSKELFGKGPQNAFFLVKLWADLNCNIQDDAGAFYRGRKRRSELQSVISHLLRVRARVADRLCGVYKVHGNYGRVRGRKRRSILSETTCGCFTFRPQTKISTQICAAKAWRKACSRGRKRRSWPRPLPLRPPRRPPLWKTSAPRPAPPRDACPCLRGRKRRSYQTRGCSQPERVKFTLSMLVTMAPIVLILLGLLRGRKRRSFHGYEAVKEALIDNGERVFWKRQFPNISKNYRGRKRRSTLLQKLLSKKPEDRPNASEILRTLTVWKKSPEKRGRKRRSSLTSLHAVDGDSKLFRTRRPRSSSDALSASFNGRGRKRRSTVRGDTTNTVLQGLNEGTQYALSVTALYASGAGRGRKRRSKHLSGEFGKPYFIKLMVFVAEERKHYTVYPPPHRGRKRRSPLASCAGLSPTSEQTTLPATVNLTVSSSSYGSQRGRKRRSQEVDRQRALQQRLEMEQHGMVGSEISSSRTSVSRGRKRRSRQSEGHSVMDTLAVALLVAEEAIEEAISKAEAYRGRKRRSLFEEKHYYKVDYSRFHNTYEVPNTPLCSARDLARGRKRRSTPVTPEMERAKRKQENISSVLYSDSFRKQIQGKRGRKRRSIVDPAVMGGFAKYEKAYFTEEYVRDHPEDQDKLRGRKRRSMILTNILSSPYFKVQLCELKTYHEVVDEIYFKVRGRKRRSWSLSGDSRLKMRTELGEYIRRITTVPLPTAPNIRGRKRRSSKYFPSSSPVNIRLSSQSVSDTFVKEVLKWKYERGRKRRSYLLSDLGPQERMHISKFTGLSMTTISHWLANVKRGRKRRSKRCSCSSLMDKECVYFSHLDIIWVNTPEHVVPYRGRKRRSKANTYEKYWPFYQKQGVHYFPKDHLKKAVAEIERGRKRRSGLPSFRDQQCQAHDRLIPKKKGLLTAVVVDDKPRGRKRRSPHLYRDGHEEALSKFTSKKLLLLVCFLDYAKISRGRKRRSLMPDKPLSANVPNDKFTQNPIPRGRKRRSYSQYSHLVGHRRVHTGEKPYRCHECGKAFNQGSRGRKRRSSDQQTVPGSEQVHEDLLISPQSSSIGSVDEGVSRGRKRRSFPNCCNTSTVADAYRWCNHTVGNRTVVEEGYYYRGRKRRSKATQRRPHGRERRVASMVSYKEESGSDEAGSGSRGRKRRSADNFQSQLAAKKGQYDGQEAHNDAMILYDKYFSRGRKRRSFFGHNAEKEKAQLAAEAQKQANRVSGSREPRPARGRKRRSMVFGEFFHRPGQDGELVNLNVGGFKQSVDQRGRKRRSGKVFSYYSCLARHRKIHTGEIPYKCNDCGKAYTRGRKRRSDEEAKTTTTNTQMEGDDEAAFLERLARREERRQRGRKRRSSRYLIRSDDMIETVYNNRGEIVKTKERRVFMLN

SEQ ID NO: 389 - protein, 1595 aa, synthetic construct
MDWTWILFLVAAATRVHSPMLTRRIGVGVAVLNRHLYAVGGFDGTNRLNSARGRKRRSGGPQALVNIMRTYTYEILLWTTSRVLKVLSVCSRGRKRRSQQEIRSIHVKRHLEPLPAGYLYNGTQFVNFFGDRGRKRRSIATMVSGSSGLAAARLLSHSFLLPQNGIRHCSYRGRKRRSYRRNIWNDAKTVSVITTACFSKVTKILVAALTFRGRKRRSEKREAVNSLQEELSNVQDHLNLAKQDLLHTTKHRGRKRRSASWYSLSKGMQDFNYLPTNCFEITLELSCDKFPRGRKRRSQELESEPRSWSLLEQLSLAGADLAAPGVQQQLERGRKRRSMQPLALRTGLVMAALSACAMPSSTHASQVPSPTRGRKRRSHDHPVLSVDCYLNLGSPISVCYVSSRPHSLNISRGRKRRSDSWKNQTFQNESRTSSSFPSVYTITSNDISVNTRGRKRRSAVRVVGDGASVDLLLLSCTWRMSAAVVGGPWRTRGRKRRSYVFIYQALLEHYLYGDTELEMTSLETHLQKIYNRGRKRRSEQTAEKPVPALSSTAWIPKEAVTPLDEQEGDGSRGRKRRSDVNVRGERGKTPLILAMEKKHLGLVQRLLEQEHRGRKRRSEVLYDSLQLAHKGILNSFYGYVMRKGARWYSMERGRKRRSTWDPPVPKCLKVLPPSNTKPPALSHSVSTSSTTRGRKRRSELSLEALAPGGLQQILSFIYTSKLLVNAANVHERGRKRRSLNLDQLKEEIAFLKHTEYKENNISTIMPFRGRKRRSGDTSTSSLLSEMSSVFRRLLPPSLDTYSECSEVRGRKRRSQETIDKLRGIVSEKTNEISNIQKDLEHSNDALKRGRKRRSPLAGFGYGLPISRLYAKYFKGDLNLYSLSGYGTRGRKRRSQSPESLLQLKALKPGVIHILGVKTSRFLCQRPDRGRKRRSGCELISTPDQDHTDFTECLKMLQKKIEEKDLKVRGRKRRSMGVLGILCFLIFLGKTWGQEQRGRKRRSCDHYWPADQDSLYYGDHILQMLSESVLPEWTIRRGRKRRSTRHKRLHTGEKTYKCGECGKAFKESSALTKHKIRGRKRRSNIEEERYSERVYAIREGETLELTCLVTGHPRPQRGRKRRSYNHLVYSQIPAAVKLTYQDAINLQNYVEEKLIPRGRKRRSSDSEEVSFIVQLVRKLMIIISRPARLLECLEFDRGRKRRSVNSKSGPEGDSKMSFSRTHSTATMPPPINPILARGRKRRSVIASYNQLSSFRQPKVMRYRVVNLYEDDHKNPTRGRKRRSSSQTLPPDSTATDITRLQPGTTYQVAVSVLRGRRGRKRRSVGDIVIIKGKEYIPADTILLSSSEPQAMCYIETRGRKRRSQETKKVLQSEGEAVDPQANVIKQNSGAATSEETRGRKRRSFTDADDVAILTYEKENARSPSSVTGNALWKAMERGRKRRSILRDTGPRRRGRRPRSKLLKAPSIVADSPSGMGRGRKRRSEWFCRINDISQRGFLVGILKQLNSLYLLHYFQNRGRKRRSLCKGAAPPDSPVVYSDSAGYNKQWHPTCFVCAKRGRKRRSQLFPMWRFPFYLSAVLCVFLYFYCVIRDVIYPY

SEQ ID NO: 390 - protein, 1607 aa, synthetic construct
MDWTWILFLVAAATRVHSDLIKSMLRNERQFKEEMLAEQLKQAEELRQYKVRGRKRRSPDRKAAVSHWQQQSYLDSGSHSGATTTAPSLSGRGRKRRSRLRKIADENGAYLLADMAHISGLVAAGVVPSPFRGRKRRSDGIERTCDTLLMCIVTMLNQGLRNGGGVGDVLRRGRKRRSAPKAEPAIIPATLNEPIGLKASDFLPAVKIPQQRGRKRRSCVPHRYAQEVSRLCLLTAGTYKVVPSTYLPDTERGRKRRSSKSESPKEPEQLRKLFLGGLSFETTDESLRSHFRGRKRRSSLPMQWLHRPLLEGRLSLNYDCFKTDFKEQDIKRGRKRRSDDYGRDLSSVQTVLTKQETFDAGLQAFQQEGIARGRKRRSEMLRFGDRMFYKDWWNYTSYSNYYRTWNVVVHDRGRKRRSSVKKEKVVTITLDKEMAFSVLLHRVWKKHPVNVRGRKRRSSDGVGAPPHSAAAALPPPSLRLRATLRPEPVCRRGRKRRSVNGDRRWITQLHFAFQEENYLYLVMEYYVGGDLRGRKRRSKLHFIIGNGILRPALRSVPESLLVAEWCLCQPSRGRKRRSFVTLEEGKGDLEEYGQYLLHTVFKNGKVTKSYSRGRKRRSSGAFLLLFLLVLTVTEPLRPKLRCNPGQFACRSRGRKRRSGKLQETQDRAKRQEDLLMKVENLALKNRFQAVLRGRKRRSLEEILPLGPNKALFYKRMASYLLPGQKSPWFFQRGRKRRSRGSLNPENPVQVSINQFTAAIYDLLRLHANSGRRGRKRRSFDTNGDGTIDFREFIIVLSVTSRGKLEQKLKWARGRKRRSQLLVQQVALGTMMHCQTLKDQPYAYSKENWEKTRGRKRRSGGNSLLGKKCFALRMARRLAKEEGWLAEHMLILRGRKRRSQVTSRDGRLLSTVIRALDTRSDGPFCRICHEGARGRKRRSKECGKTFSSRRNLRRHIVVQGGNRPYKFPIEDMRGRKRRSEEAKKKLLKDAEALSQSLEEKALAYDKLEKTKNRGRKRRSHEGRLSYMRSKKVSNCNQYRLGKKKGLPSRMVVRGRKRRSKHRCEYLHSKLAHIKRLLAEYDQRQLQAWPRGRKRRSLNWMIRMMSDLESNIVAVESVKEYSKTETEAPWRGRKRRSVITAGSDASSKLRALQYRCLVPGWYATHIERWLRGRKRRSDLKQQLLLNKLMGKDESTSRHRRSLSPILPGRHRGRKRRSIVAIENILSFMSYDEISQLRPVCKRMDLVCQRMRGRKRRSPRYRFRKRDKVLFCGRKIMRKVSQSTSSLVDTSRGRKRRSQITAVIVNRKEHNLKKGAGYHLDLFWVAILMVIRGRKRRSHLRVHSREKPFDCEECWRSFKVNTHLFRHQKLHRGRKRRSHANHALPVYVFTRPTYSRRLSGLSEMDLISTIGRGRKRRSPSRGGHMDDGGYSMNFKMSSSRGPLPVKRGPPRGRKRRSAAAPSGPAPTTSMRFTLPPGTSTNGKVLAATAPRGRKRRSTLRVVLATLGVGAASLGITCYTAQENELIPSIIRGRKRRSMSAPPVLRPPSPLLPGAAAAAAAAAALVPGSGPRGRKRRSKYNGPPFPPVASTFQPAADILDKVIERKETLEN

SEQ ID NO: 391 - DNA, 7745 bp, synthetic construct
  source 1..7745: mol_type = other DNA
gctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccgccaccatggattggacctggattctctttctcgttgcagctgctacacgcgttcatagcgccgtgagccactggcaacagcagtcctatctggactccggcatccactatggcgcaacaacaaccgccccatctctgtctggcaagggaaatcctgagcggggcagaaagcgcaggagccctgaggaggaggccagactgaagatcctggaaaccctgaagctgctgctgcagcacacatggccaagagtgtcctgcagactggtggtgctgctgaagagaggccgcaaacggaggtccctgttcctgcacctgatctccaacaaggtgtgcctgcggcacctgctgcacaagcgcaaggagacatggtgccctagaccaccacacccccaggccctgaggggcaggaaaaggaggtctatcaccatccagaacgcatacgagaagcagcacccttggctgtacctgagctccctgggcttctgctatctgatctatttcaattccatgagccagatgagaggcaggaaacggagatccctgtacctgagacagctgccagagcccctgatcagcttcagactgtacagagagctggtgggactggccaaggacagcctgaaggccgaggccgaggcacgcggcagaaaacggagatccaaggagctgtttggcaaggggcctcagaatgcctttttcctggtgaagctgtgggccgatctgaattgtaatatccaggacgacgccggagccttctatagaggccggaagcgcaggtccgagctgcagtccgtgatctcccacctgctgagggtgagggccagagtggccgataggctgtgtggcgtgtacaaggtgcacggcaattacggcagagtgagggggaggaagagaagaagcatcctgagcgaaaccacctgcggctgcttcaccttccgcccccagaccaagatctctacccagatctgcgccgcaaaggcctggaggaaggcctgctccaggggacggaaaagaaggagctggccaagacccctgccactgcgccctccaagaaggccacctctgtggaaaacctctgccccaagacccgcacctccaagagacgcatgcccatgtctgaggggacggaagcgcaggtcttatcagaccagaggctgtagccagccagagagagtgaagtttacactgagcatgctggtgacaatggcacccatcgtgctgatcctgctgggcctgctgagaggccggaagagacgcagctttcacggctatgaggccgtgaaggaggccctgatcgacaacggcgagagagtgttctggaagaggcagtttcctaacatctctaagaactacaggggacgcaagagacgctccacactgctgcagaagctgctgtccaagaagcctgaggaccgccccaacgcctccgagatcctgcgcacactgacagtgtggaagaagtctccagagaagagaggcaggaagaggagatccagcctgacctccctgcacgccgtggacggagactccaagctgttcaggacaagacggccacggtccagctccgacgcactgagcgcctcctttaatggccggggcagaaagcggcggtctacagtgaggggagataccaccaataccgtgctgcagggactgaacgagggcacacagtatgcactgtctgtgacagcactgtatgcctccggcgcaggcaggggcagaaagcggagatccaagcacctgtccggagagtttggcaagccttacttcatcaagctgatggtgttcgtggccgaggagagaaagcactacaccgtgtaccccccaccacacagaggccgcaagcggcggagcccactggccagctgtgccggactgagccctacctctgagcagaccaccctgcctgccacagtgaatctgaccgtgagctctagctcttatggatctcagaggggaaggaagcggagaagccaggaggtggataggcagcgggccctgcagcagaggctggagatggagcagcacggcatggtgggcagcgagatctcctccagccggacatctgtgtctcgcggcagaaagcggcgctccaggcagtctgagggccacagcgtgatggacacactggcagtggccctgctggtggccgaggaggccatcgaggaggccatctctaaggcagaggcctataggggaaggaagagacggagcctgtttgaggagaagcactactacaaggtggattacagcagattccacaacacatatgaggtgcctaataccccactgtgctctgcccgcgatctggccagaggcaggaagcgccggagcacacctgtgacccccgagatggagcgggcaaagaggaagcaggagaatatcagctccgtgctgtactccgattcctttagaaagcagatccaggggaagaggggcagaaaaagaaggtctatcgtggaccccgccgtgatgggcggctttgccaagtatgagaaggcctactttaccgaggagtatgtgagggatcacccagaggaccaggataagctgcgcggcagaaagaggaggagcatgatcctgacaaatatcctgtccagcccctactttaaggtgcagctgtgtgagctgaaaacctaccacgaggtggtggatgagatctactttaaggtgcgcggcaggaagagaaggagctggagcctgagcggagacagcaggctgaagatgagaaccgagctgggagagtatatcagacggatcacaaccgtgcctctgcccaccgcaccaaacatcagaggccgcaagagacgcagctccaagtatttccccagctcctctcccgtgaacatcaggctgagctctcagtccgtgagcgacacctttgtgaaggaggtgctgaagtggaagtatgagagaggccggaaacgcaggagctacctgctgtctgatctgggacctcaggagaggatgcacatctctaagtttaccggcctgagcatgaccacaatctcccactggctggcaaacgtgaagcggggcagaaagaggcgctctaagcggtgctcttgctccagcctgatggacaaggagtgcgtgtacttctctcacctggacatcatctgggtgaacacacccgagcacgtggtgccctacaggggccgcaagcgccggtctaaagcaaatacctacgagaagtattggcccttttaccagaagcagggagtgcactacttcccaaaggatcacctgaagaaggcagtggccgagatcgagcgcggacggaagaggcgcagcggactgccatcctttcgggatcagcagtgtcaggcccacgacaggctgatccctaagaagaagggcctgctgacagccgtggtggtggacgacaagcctagaggacgcaaaagaagaagcccacacctgtacagggatggccacgaggaggccctgagcaagtttacatctaagaagctgctgctgctggtgtgttttctggattacgccaagatctctagaggacgcaagcgcagaagcctgatgccagacaagcccctgtccgccaatgtgcccaacgataagttcacacagaatcccatcccaaggggccgcaagcggaggtcctattcccagtacagccacctggtgggccaccgcagggtgcacacaggcgagaagccttacaggtgccacgagtgtggcaaggcattcaaccagggatctaggggccgcaaaaggaggagctctgatcagcagaccgtgcctggctccgagcaggtgcacgaggatctgctgatctctccacagtcctctagcatcggctccgtggacgagggagtgagcagggggcgcaagaggcgctcttttccaaattgctgtaacacaagcaccgtggccgatgcctacaggtggtgcaatcacaccgtgggcaacagaacagtggtcgaggagggctattactatcggggcagaaagagaaggagcaaggccacccagagacgccctcacggacgcgagaggcgggtggcatctatggtgagctataaggaggagagcggctccgacgaggcaggctctggctccaggggccgcaagagaaggtccgccgacaacttccagtcccagctggccgccaagaagggccagtatgacggccaggaggcccacaacgatgccatgatcctgtacgataagtacttctccagaggaaggaagaggcgctctttcttcggacacaatgcagagaaggagaaggcccagctggccgcagaggcccagaagcaggccaacagggtgtccggctcccgcgagccaaggcccgcaagaggcaggaagagacggtccatggtgttcggagagtttttccaccggcccggccaggacggcgagctggtgaacctgaacgtggggggcttcaagcagtctgtggatcagcggggcaggaagagaagatctggcaaggtgttcagctattatagctgcctggccagacacagaaagatccacaccggggagatcccttacaagtgtaacgactgtggcaaagcctacacacgcggccggaagagaaggtctgacgaggaggcaaagaccaccaccaccaacacccagatggagggagatgatgaggcagcatttctggagagactggcacggagagaggagcggcggcagagaggcaggaagcggaggtctagccgctatctgatcagatctgatgacatgatcgagacagtgtataacaatcgcggcgagatcgtgaaaaccaaggagagacgggtgttcatgctgaattgataactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctt

SEQ ID NO: 392 - DNA, 7745 bp, synthetic construct
  source 1..7745: mol_type = other DNA
gctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccgccaccatggattggacatggattctctttctcgttgcagcagctacacgcgttcatagccccatgctgaccagaagaatcggagtgggcgtggccgtgctgaataggcacctgtatgccgtgggcggctttgatggaaccaacagactgaactccgcccgcggccggaagaggcggtctggaggccctcaggccctggtgaatatcatgagaacctatacatacgagatcctgctgtggacaacctccagggtgctgaaggtcctgagcgtgtgcagccggggacggaagaggaggagccagcaggagatcagatccatccacgtgaaacgccacctggagcctctgcctgccggctatctgtataacggcacccagtttgtgaatttcttcggcgacagaggcagaaaacgcaggtccatcgcaaccatggtgagcggctcctccggcctggccgccgccaggctgctgtctcacagcttcctgctgcctcagaatggcatccgccactgtagctacagaggcaggaaacgccgctcctatcggcggaatatctggaatgacgccaagaccgtgtccgtgatcacaaccgcctgttttagcaaggtgaccaagatcctggtggccgcactgacattccggggacgcaagcgcagatccgagaagagggaggccgtgaacagcctgcaggaggagctgagcaatgtgcaggaccacctgaatctggccaagcaggacctgctgcacaccacaaagcacagaggccgcaagcggagatccgcctcctggtacagcctgtccaagggaatgcaggatttcaactacctgcctacaaattgctttgagatcacactggagctgtcctgtgacaagtttcctaggggccggaagcgccgcagccaggagctggagagcgagcccaggtcctggtccctgctggagcagctgagcctggccggcgccgacctggccgcacccggcgtgcagcagcagctggagcggggcaggaagaggagaagcatgcagccactggccctgagaaccgggctggtgatggccgcactgagcgcctgcgcaatgccctcctctacccacgcctcccaggtgccttccccaacaaggggcaggaagagaaggagccacgatcaccctgtgctgtctgtggactgctatctgaatctgggcagccccatctctgtgtgctacgtgagctccaggcctcactctctgaatatcagccgcggaaggaagagaaggtccgattcctggaagaaccagacctttcagaatgagtctaggacaagcagcagcttccccagcgtgtataccatcacatctaacgacatcagcgtgaataccagaggcaggaagaggcgctccgcagtgagagtcgtgggcgatggcgcctctgtggatctgctgctgctgtcttgtacatggcggatgagcgccgcagtggtgggaggcccatggaggaccagaggacggaagcggaggtcttacgtgtttatctatcaggccctgctggagcactacctgtatggagataccgagctggagatgacctctctggagacccacctgcagaagatctataacagaggcagaaagcggagatctgagcagaccgcagagaagcccgtgcccgccctgtccagcaccgcctggattccaaaggaggccgtgacccccctggacgagcaggagggcgatggctccagaggcagaaaaaggcggtccgatgtgaacgtgagaggcgagcggggcaagacccccctgatcctggccatggagaaaaagcacctgggcctggtgcagcgcctgctggagcaggagcacaggggaaggaagaggcggtccgaggtgctgtatgattccctgcagctggcccacaagggcatcctgaattctttctacggctacgtgatgagaaagggcgccaggtggtattctatggagcgcggacggaagagaagatctacctgggacccccctgtccccaagtgtctgaaggtgctgcccccatccaataccaagcctccagccctgagccacagcgtgtctacaagctccaccacaaggggcagaaagagaagatccgagctgtccctggaggcactggcacccgggggactgcagcagatcctgtctttcatctatacatctaagctgctggtgaacgccgcaaacgtgcacgagaggggcaggaagcggaggagcctgaacctggaccagctgaaggaggagatcgcctttctgaagcacaccgagtacaaggagaataacatctctaccatcatgccattcagagggagaaagaggaggtctggcgacacatctacaagctccctgctgtccgagatgagcagcgtgttcagacgcctgctgcccccaagcctggatacctatagcgagtgctccgaggtgagggggcggaagaggagatcccaggaaaccatcgacaaactgagaggcatcgtgagcgagaaaacaaacgagatcagcaacatccagaaggatctggagcacagcaatgacgccctgaagagaggcagaaagagaaggagccccctggccggcttcgggtatggcctgccaatctctaggctgtacgccaagtactttaagggcgatctgaacctgtacagcctgtccgggtatggcaccagaggaaggaagaggagatctcagtctccagagagcctgctgcagctgaaggccctgaagccaggcgtgatccacatcctgggagtgaaaacatctagatttctgtgccagcgcccagacagaggaaggaagcggagaagcggatgcgagctgatctctacaccagatcaggaccacaccgattttacagagtgtctgaagatgctgcagaagaagatcgaggagaaggatctgaaggtgcgcggcaggaagcgccggagcatgggagtgctgggcatcctgtgctttctgatcttcctgggcaagacctggggccaggagcaacggggcagaaagcgccggtcctgtgaccactattggccagccgaccaggatagcctgtactacggcgatcacatcctgcagatgctgtctgagtccgtgctgcctgagtggacaatcagacggggcagaaaaaggagaagcacaagacacaagagactgcacaccggggagaaaacctacaagtgtggagagtgtggcaaagcattcaaggagtctagcgccctgacaaagcacaagatccgcggcagaaagcgccgcagcaacatcgaggaggagaggtactccgagagagtgtacgcaatcagagagggcgagacactggagctgacatgcctggtgaccggacacccacgcccccagcgcggcaggaagagacgctcctataaccacctggtgtactctcagatccccgcagcagtgaagctgacataccaggacgcaatcaacctgcagaactacgtggaggagaagctgatcccacgcggacggaagaggagatcttccgattctgaggaggtgtctttcatcgtgcagctggtgcggaagctgatgatcatcatctcccgccctgcaagactgctggagtgcctggagttcgaccggggcaggaaacgcaggtctgtgaacagcaagagcggacctgagggcgattccaagatgtcttttagcagaacccactccaccgcaaccatgcctccaccaatcaatcccatcctggcaagaggcagaaagaggagatctgtgatcgcctcctataaccagctgtctagctttcggcagccaaaggtgatgaggtacagagtggtgaacctgtacgaggacgatcacaagaatccaacccggggcagaaagaggaggagctcctctcagacactgcctccagattccacagcaaccgacatcaccagactgcagccaggaacaacatatcaggtggccgtgagcgtgctgaggggaagaagaggcagaaagcgcaggtctgtgggcgacatcgtgatcatcaagggaaaggagtacatccctgcagacacaatcctgctgtccagctccgagcctcaggccatgtgctatatcgagacacgcggcaggaagcggcggagccaggagaccaagaaggtgctgcagtccgagggcgaggcagtggacccacaggccaatgtgatcaagcagaactccggagccgccacctctgaggaaaccaggggcagaaagcggcgctccttcacagacgccgacgacgtggccatcctgacctacgagaaggagaacgcccgcagccccagctccgtgacaggcaatgcactgtggaaggccatggagaggggccgcaagaggcgcagcatcctgagggacaccggccctcggaggaggggacgccggcctcgctctaagctgctgaaggccccaagcatcgtggccgatagcccatctggaatgggccggggccgcaagaggaggtccgagtggttctgtcggatcaacgatatctctcagcgcggatttctggtgggcatcctgaagcagctgaattctctgtacctgctgcactacttccagaacaggggacggaagaggcgctctctgtgcaagggcgccgcaccacctgattctcctgtggtgtacagcgactccgccggctacaataagcagtggcaccccacctgcttcgtgtgcgcaaagagaggccggaagagaagaagccagctgtttccaatgtggagattccccttctatctgtccgccgtgctgtgcgtgttcctgtacttttactgtgtgatcagggatgtgatctatccatattgataactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctt

SEQ ID NO: 393 - DNA, 7781 bp, synthetic construct
  source 1..7781: mol_type = other DNA
gctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccgccaccatggattggacttggattctctttctcgttgctgctgctactcgcgttcacagtgacctgatcaagagcatgctgcggaatgagaggcagttcaaggaggagatgctggccgagcagctgaaacaggcagaggagctgaggcagtacaaggtgagaggcagaaagcgcagatccccagatagaaaggccgcagtgagccactggcagcagcagtcctacctggactccggctcccacagcggcgccaccacaaccgccccaagcctgagcggaagaggccggaagcgcagatctcggctgagaaagatcgccgacgagaatggcgcatatctgctggccgacatggcccacatctccggactggtggcagccggcgtggtgccttccccatttcgcgggaggaagaggcgctccgacggaatcgagcggacatgtgataccctgctgatgtgcatcgtcacaatgctgaatcagggcctgagaaacggcggaggagtgggcgacgtgctgcggaggggaaggaagcgcaggtctgcccctaaggccgagcccgcaatcatcccagcaaccctgaacgagccaattggactgaaggcctctgactttctgccagccgtgaagatccctcagcagagaggccgcaagaggagatcctgcgtgccccacaggtacgcccaggaggtgtctcggctgtgcctgctgaccgcaggcacctacaaggtggtgccctctacctatctgcctgataccgagcggggaaggaagagaagatcttctaagagcgagagccccaaggagccagagcagctgaggaagctgtttctgggcggactgtccttcgaaaccacagatgagtctctgagatctcacttccggggcagaaaaaggaggtctagcctgccaatgcagtggctgcacagacctctgctggagggcaggctgtctctgaattacgattgttttaagaccgatttcaaggagcaggatatcaagcgcggccgcaagaggcgctctgatgactatggcagggacctgtcctctgtgcagacagtgctgacaaagcaggagacatttgatgcaggcctgcaggcctttcagcaggagggcatcgccagagggaggaagcggagatccgagatgctgagattcggcgacagaatgttctacaaggattggtggaattacacctcctattccaattactacaggacctggaatgtggtggtgcacgaccggggcagaaagagacgcagctccgtgaagaaggagaaggtggtgacaatcaccctggataaggagatggccttcagcgtgctgctgcacagagtgtggaagaagcaccctgtgaatgtgcgcggcaggaagagaagaagctccgatggcgtgggagcccctccccacagcgccgccgccgccctgcccccaccttccctgcggctgcgggccaccctgcgcccagagcctgtgtgcagacgcggcagaaagcggaggagcgtgaacggcgatcgcagatggatcacccagctgcactttgcctttcaggaggagaactatctgtatctggtgatggagtactacgtgggcggcgatctgaggggccgcaagcgccgctctaagctgcactttatcatcggcaacggaatcctgaggcctgccctgagatccgtgcctgagtctctgctggtggccgagtggtgcctgtgccagccttctcggggccgcaagcggaggagcttcgtgaccctggaggagggcaagggcgacctggaggagtatggccagtatctgctgcacacagtctttaaaaacggaaaggtgacaaagtcctactcccgcgggcggaagcggagatctagcggcgccttcctgctgctgtttctgctggtgctgaccgtgaccgagcccctgcgcccaaagctgaggtgcaatccaggccagtttgcctgtaggagcagaggacgcaagcggagatctggcaagctgcaggagacccaggacagggccaagaggcaggaggacctgctgatgaaagtggagaatctggcactgaagaataggttccaggcagtgctgagaggcaggaaaagaaggagcctggaggagatcctgccactgggaccaaataaggccctgttttataagaggatggcatcctatctgctgcctggccagaagtccccctggttcttccagagaggccggaagaggcggagcaggggctctctgaatccagagaaccccgtgcaggtgagcatcaaccagttcaccgccgcaatctacgatctgctgagactgcacgccaattctggcaggagaggccggaagcggaggtcttttgataccaacggagatggaaccatcgacttccgggagtttatcatcgtgctgtctgtgacatccagaggaaagctggagcagaagctgaagtgggccaggggcagaaagaggaggagccagctgctggtgcagcaggtggccctgggcaccatgatgcactgccagacactgaaggaccagccttatgcctacagcaaggagaactgggagaaaacaagaggccgcaagcgcagatccggaggcaactctctgctgggcaagaagtgtttcgccctgcgcatggcaagacgcctggccaaggaggagggctggctggcagagcacatgctgatcctgagaggcaggaagaggcggagccaggtgacatctagagatggcagactgctgagcacagtgatcagggcactggatacaagatccgacggcccattctgtaggatctgccacgagggggcacgcggcagaaagagacggtccaaggagtgtggcaagaccttcagctctagacggaacctgaggagacacatcgtggtgcagggcggcaataggccatataagttcccaatcgaggacatgagaggcagaaagagaaggtccgaggaggccaagaagaagctgctgaaggacgccgaggcactgagccagagcctggaggagaaggcactggcctacgataagctggagaaaaccaaaaatcggggcaggaagaggagatctcacgaggggcgcctgagctacatgagatctaaaaaggtgagcaactgtaaccagtataggctggggaagaagaagggcctgcctagcagaatggtggtgcgcggcagaaagcgcaggtccaagcacagatgcgagtatctgcactctaagctggcacacatcaagcgcctgctggcagagtatgaccagagacagctgcaggcatggcctcgcggccgcaagcggagatccctgaactggatgatcagaatgatgtccgatctggagtccaacatcgtggccgtggagtccgtgaaggagtactctaagaccgagaccgaggcaccctggaggggcaggaagcggagaagcgtgatcacagccggctccgacgccagctccaagctgagggccctgcagtaccggtgcctggtgcctggctggtatgcaacacacatcgagcgctggctgaggggccggaaaagaaggtctgacctgaagcagcaactgctgctgaataagctgatgggcaaggatgagtccacctcccggcatcggcgcagcctgagcccaatcctgcctggaaggcaccgcggcaggaagcgcagaagcatcgtggcaatcgagaacatcctgagctttatgtcctacgacgagatcagccagctgaggccagtgtgtaagcggatggacctggtgtgccagagaatgcggggaaggaaaaggcggagccccaggtacagattcaggaagagagataaggtgctgttttgcggcaggaagatcatgcggaaggtgtcccagtccacctcctccctggtggatacaagcaggggcagaaagcggagaagccagatcacagccgtgatcgtgaaccgcaaagagcacaacctgaagaagggcgcaggataccacctggacctgttctgggtggccatcctgatggtgatccgcggcaggaaacggagaagccacctgagagtgcacagcagggagaagccattcgactgcgaggagtgttggaggtccttcaaggtgaacacccacctgtttagacaccagaagctgcacagaggccggaagagacggtcccacgcaaatcacgccctgcctgtgtatgtgttcacaaggcccacatactctaggaggctgtctggcctgtccgagatggatctgatctctacaatcggaagaggacggaaaagaaggtccccctcccgcggaggccacatggacgacggcggatacagcatgaactttaagatgagctcctccagaggccctctgccagtgaagagagggccacctcgcggccggaagaggaggtctgccgccgcaccaagcggaccagcccccaccaccagcatgcgcttcacactgcccccaggaacctctacaaacggcaaggtgctggccgcaaccgcccccaggggccggaagagaaggagcacactgcgcgtggtgctggccaccctgggagtgggagccgcaagcctgggcatcacctgttataccgcccaggagaacgagctgatcccctctatcatccgcggacgcaagcgccgcagcatgtctgccccccctgtgctgaggccccccagccccctgctgccaggcgcagccgccgcagccgcagccgccgccgcactggtgcccggatctggccccagaggcagaaagaggcggagcaagtacaatggacctccattccctccagtggccagcacatttcagccagccgccgacatcctggataaggtgatcgagaggaaggaaaccctggagaattgataactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttcttgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctt

SEQ ID NO: 394 - protein, 15 aa, synthetic construct
DSGIHSGATTTAPSL

(19) United States
(12) Patent Application Publication
(10) Pub. No.: US 2026/0345754 A1
(43) Pub. Date: Nov. 5, 2026

(54) VACCINES AND METHODS OF USING THE SAME TO TREAT WNT-RELATED CANCER

(71) Applicant: Geneos Therapeutics, Inc., Philadelphia, PA (US)

(72) Inventors: Alfredo Perales-Puchalt, Valencia (ES); Niranjan Sardesai, Blue Bell, PA (US)

(21) Appl. No.: 19/667,400

(22) Filed: May 4, 2026

Related U.S. Application Data
(63) Continuation of application No. 18/865,104, filed on May 10, 2023.
(60) Provisional application No. 63/340,090, filed on May 10, 2022.
(60) Provisional application No. 63/342,605, filed on May 16, 2022.

(51) Int. Cl. A61K 39/00; A61K 38/20; A61K 39/395; A61P 35/00

(52) U.S. Cl. CPC A61K 39/0011; A61K 38/208; A61K 39/39558; A61P 35/00; A61K 2039/505; A61K 2039/53

(57) ABSTRACT

The present disclosure provides a composition comprising a nucleic acid sequence encoding from about 1 to about 100 amino acid sequences that are tumor-specific antigens, wherein at least one tumor-specific antigen is an amino acid sequence associated with a WNT pathway. In some embodiments, the nucleic acid sequence encodes from about 20 to about 60 tumor-specific antigens; wherein from about 1 to about 8 tumor-specific antigens are chosen from one or a combination of: WNT, CTNNB1, AXIN1, AXIN2, APC, CK1, and GSK3B. Also provided is a cell comprising any one or plurality of compositions disclosed herein. Also provided is a pharmaceutical composition comprising: (i) a therapeutically effective amount of one or a plurality of compositions as disclosed herein; and (ii) a pharmaceutically acceptable carrier. Also provided is a method of treating cancer in a subject in need thereof comprising administering to the subject: (i) a pharmaceutical composition comprising a nucleic acid sequence encoding about twenty or more neoantigens or epitopes specific for neoantigens; or (ii) a pharmaceutical composition disclosed herein. In some embodiments, the method further comprises administering to the subject a therapeutically effective amount of a checkpoint inhibitor.

### CROSS-REFERENCE TO RELATED APPLICATIONS

This application is a continuation application of U.S. application Ser. No. 18/865,104, filed on Nov. 12, 2024, which is a U.S. National Stage Application of International Application No. PCT/US2023/066856, filed on May 10, 2023, which claims the benefit of U.S. Application No. 63/342,605, filed on May 16, 2022 and U.S. Application No. 63/340,090, filed on May 10, 2022, the contents of which are hereby incorporated by reference in their entireties.

### REFERENCE TO SEQUENCE LISTING

The Sequence Listing submitted May 10, 2023 as an XML file named “GENE 003_PCT_SL.xml” created on May 10, 2023 and having a size of 526,450 bytes is hereby incorporated by reference pursuant to 37 C.F.R. § 1.52 (e) (5).

### FIELD

The disclosure generally relates to compositions and vaccines comprising antigens associated with the beta-catenin pathway as well as methods of treating beta-catenin altered cancer in a subject with cancer by use of cancer vaccines.

### BACKGROUND

WNTs are secreted cysteine-rich and lipid-modified growth factors that bind Frizzled receptors and LRP5/6 co-receptors on the surface of cells receiving the Wnt signal. Intracellular signal transduction involves the stabilization of β-catenin. In the absence of Wnt, the β-catenin destruction complex resides in the cytoplasm, where it binds, phosphorylates, and ubiquitinates β-catenin, leading to its degradation within the proteasome. Wnt induces the association of the intact complex with phosphorylated LRP5/6. After binding to LRP5/6, the destruction complex captures and phosphorylates β-catenin, but ubiquitination by βTrCP is blocked. Newly synthesized β-catenin accumulates, translocates to the nucleus and associates with TCF family transcription factors to initiate transcription of Wnt target genes (Clevers and Nusse, 2012).

### SUMMARY

The present disclosure provides a composition comprising a nucleic acid sequence encoding from about 1 to about 100 amino acid sequences that are tumor-specific antigens, wherein at least one tumor-specific antigen is an amino acid sequence associated with a WNT pathway.

In some embodiments, the composition further comprises a nucleic acid molecule, wherein the nucleic acid molecule comprises the nucleic acid sequence encoding the antigens; wherein the nucleic acid molecule comprises a regulatory sequence operably linked to the nucleic acid sequence encoding tumor-specific antigens; wherein the nucleic acid sequence encodes from about 20 to about 60 tumor-specific antigens, each tumor-specific antigen flanked by at least one linker on a contiguous amino acid sequence, and wherein the nucleic acid sequence encodes a leader sequence on the 5′ end of the first antigen sequence in the 5′ to 3′ orientation. In some embodiments, the composition further comprises a nucleic acid molecule, wherein the nucleic acid molecule comprises the nucleic acid sequence encoding the antigens; wherein the nucleic acid molecule comprises a regulatory sequence operably linked to the nucleic acid sequence encoding tumor-specific antigens; wherein the nucleic acid sequence encodes from about 1 to about 60 tumor-specific antigens, each antigen flanked by at least one linker on a contiguous amino acid sequence, and wherein the nucleic acid sequence encodes a leader sequence on the 5′ end of the first antigen sequence in the 5′ to 3′ orientation. In some embodiments, the composition further comprises a nucleic acid molecule, wherein the nucleic acid molecule comprises the nucleic acid sequence encoding the antigens; wherein the nucleic acid molecule comprises a regulatory sequence operably linked to the nucleic acid sequence encoding tumor-specific antigens; wherein the nucleic acid sequence encodes at least about 20 tumor-specific antigens, each antigen flanked by at least one linker on a contiguous amino acid sequence, and wherein the nucleic acid sequence encodes a leader sequence on the 5′ end of the first antigen sequence in the 5′ to 3′ orientation.

In some embodiments, the tumor-specific antigens are chosen from one or a combination of: WNT, CTNNB1, AXIN1, AXIN2, APC, CK1, and GSK3B.

In some embodiments, the nucleic acid sequence comprises a nucleic acid sequence encoding β-catenin or a fragment thereof, wherein the nucleic acid sequence encoding the β-catenin or fragment thereof comprises at least about 70% sequence identity to SEQ ID NO:1, SEQ ID NO:3, SEQ ID NO:5, SEQ ID NO:7, SEQ ID NO:9, or SEQ ID NO: 11.

In some embodiments, the antigen expression domain comprises a nucleic acid encoding β-catenin or a fragment thereof, wherein the nucleic acid sequence encoding the β-catenin or a fragment thereof comprises at least about 70%, 75%, 80%, 85%, 90%, 91%, 92%, 93%, 94%, 95%, 96%, 97%, 98%, 99% sequence identity to SEQ ID NO:1, SEQ ID NO:3, SEQ ID NO: 5, SEQ ID NO:7, SEQ ID NO:9, or SEQ ID NO:11.

In some embodiments, wherein the tumor-specific antigen is free of a nucleic acid sequence that comprises 100% SEQ ID NO:1, SEQ ID NO:3, SEQ ID NO:5, SEQ ID NO:7, SEQ ID NO:9, or SEQ ID NO:11.

In some embodiments, the linkers are P2A linker sequences or furin linker sequences.

In some embodiments, the nucleic acid sequence encodes from about 20 to about 60 tumor-specific antigens; wherein from about 1 to about 8 tumor-specific antigens are chosen from one or a combination of: WNT, CTNNB1, AXIN1, AXIN2, APC, CK1, and GSK3B.

In some embodiments, the nucleic acid sequence encodes, in a 5′ to 3′ prime orientation, a leader sequence and one or a plurality of tumor-specific antigens, wherein the one or plurality of tumor antigens are flanking at least one linker sequence.

Also provided is a cell comprising any one or plurality of compositions disclosed herein.

Also provided is a nucleic acid molecule comprising any one or plurality of compositions disclosed herein.

Also provided is a pharmaceutical composition comprising: (i) a therapeutically effective amount of one or a plurality of compositions as disclosed herein; and (ii) a pharmaceutically acceptable carrier.

In some embodiments, the composition comprises a plasmid comprising an expressible nucleic acid sequence that encodes from about 40 to about 60 tumor-specific antigens.

In some embodiments, the pharmaceutical composition comprises a therapeutically effective amount of pVAX0001 comprising a nucleic acid sequence that encodes from about 1 to about 7 tumor-specific antigens are chosen from one or a combination of: WNT, CTNNB1, AXIN1, AXIN2, APC, CK1, and GSK3Bh. In some embodiments, the pharmaceutical composition comprises a therapeutically effective amount of a nucleic acid molecule disclosed herein comprising a nucleic acid sequence that encodes from about 20 to about 55 tumor-specific antigens are chosen from one or a combination of epitopes disclosed in the Examples, but wherein the tumor-specific epitopes are free of a WNT pathway epitope. In some embodiments, the pharmaceutical composition comprises a therapeutically effective amount of nucleic acid molecule disclosed herein comprising a nucleic acid sequence that encodes from about 20 to about 75 tumor-specific antigens are chosen from one or a combination of epitopes disclosed in the Examples. In some embodiments, the pharmaceutical composition comprises a therapeutically effective amount of pVAX0001 comprising a nucleic acid sequence that encodes from about 20 to about 55 tumor-specific antigens are chosen from one or a combination of epitopes disclosed in the Examples. In some embodiments, the tumor-specific antigens are encoded by one or a plurality of nucleic acid sequences that encodes amino acid sequences chosen from one or a combination of SEQ ID NO: 70 through 286, or functional fragments that comprise at least about 70%, 75%, 80%, 85%, 86%, 87%, 88%, 89%, 90%, 91%, 92%, 93%, 94%, 95%, 96%, 97%, 98%, or 99% sequence identity to the amino acids chosen from one or a combination of SEQ ID NO: 70 through 286.

Also provided is a method of treating cancer in a subject in need thereof comprising administering to the subject: (i) a pharmaceutical composition comprising a nucleic acid sequence encoding about twenty or more neoantigens or epitopes specific for a neoantigens; or (ii) a pharmaceutical composition disclosed herein.

In some embodiments, the method further comprises administering to the subject a therapeutically effective amount of a checkpoint inhibitor.

In some embodiments, the checkpoint inhibitor is chosen from one or a combination of the checkpoint inhibitors of Table 1. In some embodiments, the therapeutically effective amount of a checkpoint inhibitor is from about 150 mg to about 250 mg.

In some embodiments, the dose of the pharmaceutical composition is from about 0.3 to about 3 milligram per subject.

In some embodiments, the step of administering is accomplished by intravenous injection, intramuscular injection, intraperitoneal injection or intradermal injection following transfection of cells by electroporation.

In some embodiments, the pharmaceutical composition comprises an expressible nucleic acid sequence comprising Formula I: [(AEDn)-(linker)]n-[AEDn+1], wherein the AED is an independently selectable antigen expression domain, wherein AEDn is a first antigen expression domain and wherein AEDn+1 is a second antigen expression domain; wherein each linker is independently selectable from about 0 to about 300 natural or non-natural nucleic acids in length, wherein the antigen expression domain 1 is independently selectable from about 12 to about 15,000 nucleotides in length and encodes a neoantigenic epitope; wherein the antigen expression domain 2 is independently selectable from about 12 to about 15,000 nucleotides in length and encodes a neoantigenic epitope; and wherein n is any positive integer from about 19 to about 500.

Also provided is a method of preventing resistance to checkpoint inhibitor therapy in a subject comprising administering to the subject in need thereof: (i) the pharmaceutical composition as disclosed herein; (ii) a pharmaceutical composition comprising a nucleic acid sequence encoding about twenty or more neoantigens or epitopes specific for a neoantigens.

In some embodiments, the composition comprises a plasmid comprising an expressible nucleic acid sequence that encodes from about 40 to about 60 tumor-specific antigens.

In some embodiments, the pharmaceutical composition comprises a therapeutically effective amount of pGX0001 comprising a nucleic acid sequence that encodes from about 1 to about 7 tumor-specific antigens are chosen from one or a combination of: WNT, CTNNB1, AXIN1, AXIN2, APC, CK1, and GSK3Bh.

In some embodiments, wherein the subject has cancer characterized by high tumor load.

In some embodiments, the subject has cancer characterized by one or more mutations in WNT, CTNNB1, AXIN1, AXIN2, APC, CK1, and GSK3Bh.

In some embodiments, the subject has cancer characterized by one or a combination of aberrant regulation of expression of WNT, CTNNB1, AXIN1, AXIN2, APC, CK1, and GSK3Bh.

In some embodiments, the subject has cancer characterized by hyper-amplification of one or a combination of amino acid sequences comprising at least 70% sequence identity to WNT, CTNNB1, AXIN1, AXIN2, APC, CK1, and GSK3Bh.

In some embodiments, the dose of the pharmaceutical composition is from about 0.3 to about 3 milligram per subject.

In some embodiments, the step of administering is accomplished by intravenous injection, intramuscular injection, intraperitoneal injection or intradermal injection following transfection of cells by electroporation.

In some embodiments, the subject has a cancer characterized by dysfunction of the WNT pathway. In some embodiments, the subject has hepatocellular cancer.

Also provided is a method of inducing an immune response in a cell comprising exposing the cell to one or more compositions disclosed herein. In some embodiments, the step of exposing is accomplished in vivo.

Also provided is a method of enhancing a CD8+ T cell response in a subject comprising administering to the subject a pharmaceutical composition disclosed herein. In some embodiments, the CD8+ T cell response is enhanced from about 10 to about 15% as compared to the CD8+ T cell response of a subject untreated with the pharmaceutical composition.

### BRIEF DESCRIPTION OF DRAWINGS

FIG. 1A is a diagram depicting the elements of the pGX0001 plasmid, including the multiple cloning site. FIG. 1B is a diagram depicting the elements of the pGX6001 plasmid comprising the IL-12 alpha and the IL-12 beta subunits.

FIG. 2 depicts β-catenin activation.

FIG. 3 is a chart depicting immune system involvement in hepatocellular carcinomas. The chart shows that mutations in the WNT pathway result in primary resistance to checkpoint inhibitors.

FIG. 4 (top) depicts the progression free survival of HCC patients with WNT unaltered pathways and HCC patients with WNT activated pathways when treated with a checkpoint inhibitor or sorafenib. WNT activation results in resistance to checkpoint inhibitors. (Harding et al., Clin Cancer Res. 2019) FIG. 4 (bottom) is a dot plot showing progression free survival or overall survival of patients treated with either TKI or immunotherapy. Wnt activation results in resistance to immunotherapy.

FIG. 5 is a line graph plotting progression free survival in HCC patients with WNT unaltered pathways and HCC patients with WNT activated pathways when treated with GNOS-PV02 and pembrolizumab. HCC patients with WNT activated pathways were not resistant to anti-PD1 therapy when treated in combination with GNS-PV02.

FIG. 6A depicts the percentage change in the size of the lesion over time in HCC patients with WNT unaltered pathways and HCC patients with WNT activated pathways. FIG. 6B and FIG. 6C shows percentage change in the size of the lesion over time and the overall response rate (ORR) in HCC patients with WNT activated cancer (50%; FIG. 6B) and WNT unaltered cancer (15.4%; FIG. 6C) to treatment with GNOS PV-02 in combination with anti-PD1 therapy.

FIG. 7 is a table showing the specific mutations in the WNT pathway genes CTNNB1 and AXIN1 found in the 6 HCC patients who were treated with GNOS-PV02 in combination with anti-PD1 and the response (PR, partial response) (SD, stable disease) (PD, progressive disease).

FIG. 8 is a dot plot showing the number of neoantigens (FIG. 8A) or the tumor mutational burden (TMB) (FIG. 8B) in HCC patients having activated WNT pathway or HCC patients having unaltered WNT pathways. WNT activated tumors have a higher number of neoantigens and a higher TMB than unaltered WNT tumors.

FIG. 9 depicts gene expression (measured in RNA transcripts per million) of AKR1C2, ABCC2, ALDH1L1, ALD3A2, GCLC and GCLM. HCC patients having β-catenin and Axin1 mutations overexpress AKR1C2, ABCC2, ALDH1L1, ALD3A2, GCLC and GCLM.

FIG. 10 depicts the percentage of cancers (y-axis) that have an alteration (mutations structural variant, amplification, or deep deletion) in CTNNB1 in a variety of tumor types (x-axis). Data was obtained from the CBIOportal database.

FIG. 11 depicts the mutation count (FIG. 11A) or the TMB (FIG. 11B) in HCC patients having altered CTNNB1 or HCC patients having unaltered CTNNB1.

FIG. 12 depicts progression free survival (PFS) upon sorafenib treatment (FIG. 12A) or immunotherapy treatment (FIG. 12B) in HCC patients having altered CTNNB1 or HCC patients having unaltered CTNNB1. CTNNB1 mutated HCC responds to TKI therapy but not CPI.

FIG. 13 depicts overall survival upon sorafenib treatment (FIG. 13A) or immunotherapy treatment (FIG. 13B) in HCC patients having altered CTNNB1 or HCC patients having unaltered CTNNB1.

FIG. 14 depicts the percentage of cancers (y-axis) that have an alteration (mutations structural variant, amplification, or deep deletion) in AXIN1 in a variety of tumor types (x-axis). Data was obtained from the CBIOportal database.

FIG. 15 depicts the percentage of cancers (y-axis) that have an alteration (mutations structural variant, amplification, or deep deletion) in AXIN2 in a variety of tumor types (x-axis). Data was obtained from the CBIOportal database.

FIG. 16 depicts the percentage of cancers (y-axis) that have an alteration (mutations structural variant, amplification, or deep deletion) in APC in a variety of tumor types (x-axis). Data was obtained from the CBIOportal database.

FIG. 17 depicts the percentage of cancers (y-axis) that have an alteration (mutations structural variant, amplification, or deep deletion) in CSNK1A1 in a variety of tumor types (x-axis). Data was obtained from the CBIOportal database.

FIG. 18 depicts the percentage of cancers (y-axis) that have an alteration (mutations structural variant, amplification, or deep deletion) in GSK3B in a variety of tumor types (x-axis). Data was obtained from the CBIOportal database.

FIG. 19 is a bar graph showing Elispot responses at screening, week 3, week 6 and week 9 following administration of neoantigen vaccine in patient PT1.

FIG. 20 is a bar graph showing Elispot responses at screening, week 3, week 6, week 9 and week 12 following administration of neoantigen vaccine in patient PT6.

FIG. 21 is a bar graph showing Elispot responses at screening, week 3, week 6, week 9 and week 12 following administration of neoantigen vaccine in patient PT2.

FIG. 22 is a bar graph showing Elispot responses at screening, week 3, week 6 and week 9 following administration of neoantigen vaccine in patient PT4.

FIG. 23 is a flow cytometry plot showing strong a CD8 T-cell response in patient PT6 (measured by CD69 and Ki67).

FIG. 24 is a flow cytometry plot showing a strong CD4 T-cell response in patient PT6 (measured by CD69 and Ki67).

FIG. 25A is a dot plot showing overall response (Y-axis) compared to the number of neoantigens (X-axis) in 19 patients with advanced HCC treated with a personalized cancer vaccine (PCV)+pIL12+Pembrolizumab in a 2nd line setting. FIG. 25B is a chart numerically depicting the data in FIG. 25A. Tumor size was dramatically reduced after administration of PCV encoding at least 20 neoantigens as compared to plasmid encoding less than 20 neoantigens.

FIG. 26A is plot showing overall change in lesion size from baseline (%) over time in 7 patients with advanced HCC treated with PCV (<20 neoantigens)+pIL12+Pembrolizumab in a 2nd line setting. FIG. 26B is plot showing overall change in lesion size from baseline (%) over time in 12 patients with advanced HCC treated with PCV (<20 neoantigens)+pIL12+Pembrolizumab in a 2nd line setting.

FIG. 27A is a non-limiting example of a manufacturing process for personalized DNA vaccines. Needle-to-needle has been achieved in as low as 6 weeks and can be regularly achieved in 6-8 weeks. FIG. 27B is a non-limiting example of a clinical trial design.

FIG. 28A shows a spider plot showing the first 12 patients of the clinical trial at the time of the data cut. FIG. 28B is a waterfall plot showing the best overall response achieved by the first 12 subjects of the clinical trial at the time of the data cut. Best overall response shows 25% partial response rate and 67% Disease Control Rate. FIG. 28C is tumor imaging scans (day 0 vs week 27 post-treatment) of patients categorized as PR. Red arrows point at the tumors.

FIG. 29A shows a bar graph that all patients analyzed to date (n=10) have newly detected and expanded T cell clones after treatment with GNOS-PV02. FIG. 29B shows a cumulative frequency of expanded clones in peripheral blood (PBMC, left) and in the tumor tissue (right) pre-vs post-vaccination (week 9) per patient. FIG. 29C shows expansion of pre-vaccination clones (dots along the X axis) and detection of multiple new T cell clones (dots along Y axis) post-vaccination in blood and tumor tissue from subject Pt 7. Arrows highlight infiltration of high frequency clones from blood into the tumor 9 weeks post-vaccination (only top 6 clones shown for clarity). Most abundant clones show an active phenotype (CD8+CD69+) as assessed by TCRβ and RNA sequencing. Approx. 75% of new TIL clones were undetectable in blood prior to vaccination.

FIG. 30A shows patient-specific clonal TCR sequences were gene optimized using GOAL algorithm and inserted into the pMXs-IRES-GFP retroviral plasmid vector containing viral packaging signal, transcriptional and processing elements, and GFP reporter gene. FIG. 30B shows an example of anti-tumor specific T cell reactivity post-vaccination evaluated by ELISpot (subject PT 8). PBMCs were stimulated with a pool of, or individual peptides encoded in the personalized GNOS-PV02 treatment. FIG. 30C shows representative images of activated, GFP positive, CD8 and CD4 TCR-engineered T cells (subject PT 8), stimulated with ATP1A1-ALB (10 μg/mL). TNTC, Too Numerous To Count; EOT, End of Treatment.

### DETAILED DESCRIPTION

The disclosure relates to methods of treating cancer by use of cancer vaccines. In some embodiments, the cancer is characterized by dysfunction in the WNT phenotype. In some embodiments, the cancer is characterized by a dysfunction in WNT signaling. In some embodiments, the dysfunction in WNT signaling is caused by 1) cells that express modulators of Axin destabilization and/or TNKS pathway dysfunction. In some embodiments, the cancer is characterized by one or more mutations in one or more WNT pathway signaling molecules. In some embodiments, the cancer is characterized by one or more mutations in WNT, CTNNB1, Axin1, Axin2, APC, CK1, and/or GSK3B. Examples of the methods of the disclosure are described in detail in the Examples and Figures section of the present disclosure.

Various terms relating to the methods and other aspects of the present disclosure are used throughout the specification and claims. Such terms are to be given their ordinary meaning in the
art unless otherwise indicated. Other specifically defined terms are to be construed in a manner consistent with the definition provided herein.

The term “about” as used herein when referring to a measurable value such as an amount,
a temporal duration, and the like, is meant to encompass variations of ±20%, ±10%, ±5%, ±1%, ±0.5%, or ±0.1% from the specified value, as such variations are appropriate to perform the disclosed methods.

The indefinite articles “a” and “an,” as used herein in the specification and in the claims,
unless clearly indicated to the contrary, should be understood to mean “at least one.”

The phrase “and/or,” as used herein in the specification and in the claims, should be
understood to mean “either or both” of the elements so conjoined, i.e., elements that are
conjunctively present in some cases and disjunctively present in other cases. Other elements may
optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, “either,” “one of,” “only one of,” or “exactly one of” “Consisting essentially of,” when used in the claims, shall have its ordinary meaning as used in the field of patent law.

As used herein, the terms “activate,” “stimulate,” “enhance” “increase” and/or “induce” (and like terms) are used interchangeably to generally refer to the act of improving or increasing, either directly or indirectly, a concentration, level, function, activity, or behavior relative to the natural, expected, or average, or relative to a control condition. “Activate” refers to a primary response induced by ligation of a cell surface moiety. For example, in the context of receptors, such stimulation entails the ligation of a receptor and a subsequent signal transduction event. Further, the stimulation event may activate a cell and upregulate or downregulate expression or secretion of a molecule. Thus, ligation of cell surface moieties, even in the absence of a direct signal transduction event, may result in the reorganization of cytoskeletal structures, or in the coalescing of cell surface moieties, each of which could serve to enhance, modify, or alter subsequent cellular responses.

As used herein, the terms “activating CD8+ T cells” or “CD8+ T cell activation” refer to a process (e.g., a signaling event) causing or resulting in one or more cellular responses of a CD8+ T cell (CTL), selected from: proliferation, differentiation, cytokine secretion, cytotoxic effector molecule release, cytotoxic activity, and expression of activation markers. As used herein, an “activated CD8+ T cell” refers to a CD8+ T cell that has received an activating signal, and thus demonstrates one or more cellular responses, selected from proliferation, differentiation, cytokine secretion, cytotoxic effector molecule release, cytotoxic activity, and expression of activation markers. Suitable assays to measure CD8+ T cell activation are known in the art and are described herein.

As used herein, the term “adjuvant” is meant to refer to any molecule added to the DNA plasmid vaccines described herein to enhance the immunogenicity of the antigens encoded by the DNA plasmids and the encoding nucleic acid sequences described hereinafter.

As used herein an “antigen” is meant to refer to any substance that will elicit an immune response upon exposure to an antigen presenting cell or other immune cell capable of initiating.

As used herein, the term “anti-tumor response” refers to an immune system response including but not limited to activating T-cells to attack an antigen or an antigen presenting cell.

The terms “cancer” and “cancerous” as used herein refer to or describe a physiological condition in mammals in which a population of cells are characterized by unregulated cell growth. Thus, the term “cancer” refers to a group of diseases involving abnormal cell growth with the potential to invade or spread to other parts of the body. Examples of cancer include, but not limited to, lung cancer, bone cancer, blood cancer, chronic myelomonocytic leukemia (CMML), bile duct cancer, cervical cancer, liver cancer, pancreatic cancer, skin cancer, cancer of the head and neck, cancer of the eye, cutaneous or intraocular melanoma, uterine cancer, ovarian cancer, rectal cancer, cancer of the anal region, stomach cancer, colon cancer, breast cancer, testicular cancer, gynecologic tumors (e.g., uterine sarcomas, carcinoma of the fallopian tubes, carcinoma of the endometrium, carcinoma of the cervix, carcinoma of the vagina or carcinoma of the vulva), Hodgkin's disease, cancer of the esophagus, cancer of the small intestine, cancer of the endocrine system (e.g., cancer of the thyroid, parathyroid or adrenal glands), sarcomas of soft tissues, cancer of the urethra, cancer of the penis, prostate cancer, chronic or acute leukemia, solid tumors of childhood, lymphocytic lymphomas, cancer of the bladder, cancer of the kidney or ureter (e.g., renal cell carcinoma, carcinoma of the renal pelvis), or neoplasms of the central nervous system (e.g., primary CNS lymphoma, spinal axis tumors, brain stem gliomas or pituitary adenomas)

Specific examples of cancer include, but are not limited to, Acute Lymphoblastic Leukemia, Adult; Acute Lymphoblastic Leukemia, Childhood; Acute Myeloid Leukemia, Adult; Adrenocortical Carcinoma; Adrenocortical Carcinoma, Childhood; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood: Carcinoid Tumor, Childhood; Carcinoid Tumor, Gastrointestinal; Carcinoma, Adrenocortical; Carcinoma, Islet Cell; Carcinoma of Unknown Primary; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ Cell Tumor, Childhood; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma. Childhood Brain Stem; Glioma. Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer; Hypothalamic and Visual Pathway Glioma, Childhood; Intraocular Melanoma; Islet Cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS-Related; Lymphoma, Central Nervous System (Primary); Lymphoma, Cutaneous T-Cell; Lymphoma, Hodgkin's, Adult; Lymphoma, Hodgkin's; Childhood; Lymphoma, Hodgkin's During Pregnancy; Lymphoma, Non-Hodgkin's, Adult; Lymphoma, Non-Hodgkin's, Childhood; Lymphoma, Non-Hodgkin's During Pregnancy; Lymphoma, Primary Central Nervous System; Macroglobulinemia, Waldenstrom's; Male Breast Cancer; Malignant Mesothelioma, Adult; Malignant Mesothelioma, Childhood; Malignant Thymoma; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplasia Syndromes; Myelogenous Leukemia, Chronic; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Neurofibroma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood′, Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland Cancer, Childhood; Sarcoma, Ewing's Family of Tumors; Sarcoma, Kaposi's; Sarcoma (Osteosarcoma)/Malignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma, Malignant; Thyroid Cancer; Thyroid Cancer, Childhood; Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer; Visual Pathway and Hypothalamic Glioma, Childhood; Vulvar Cancer; Waldenstrom's Macro globulinemia; and Wilms' Tumor.

The term “checkpoint inhibitor” as used herein is meant to refer to any small molecule chemical compound, antibody, nucleic acid molecule, or polypeptide, or fragments thereof, that inhibits the inhibitory pathways, allowing more extensive immune activity. In some embodiments, the checkpoint inhibitor is an inhibitor of the programmed death-1 (PD-1) pathway, for example an anti-PD1 antibody, such as, but not limited to Nivolumab. In other embodiments, the checkpoint inhibitor is an antibody that binds or associates with cytotoxic T-lymphocyte-associated antigen (CTLA-4). In further additional embodiments, the checkpoint inhibitor is targeted at a member of the TNF superfamily such as CD40, OX40, CD 137, GITR, CD27 or TIM-3. In some embodiments, targeting a checkpoint inhibitor is accomplished with an inhibitory antibody or similar molecule. In other cases, it is accomplished with an agonist for the target; examples of this class include the stimulatory targets OX40 and GITR. In some embodiments the checkpoint inhibitor is meant to refer to any one or combination of checkpoint inhibitors from Table 1.

TABLE 1
List of Checkpoint Inhibitors
FDA Approved Checkpoint Inhibitors

| | |
|---|---|
| Pembrolizumab | Spartalizumab |
| Nivolumab | Camrelizumab |
| Cemiplimab | Sintilimab |
| Dostaslimab | Tislelizumab |
| Atezolizumab | Toripalimab |
| Avelumab | Tremelimumab |
| Durvalumab | |
| Ipilimumab | |
| Relatlimab | |

The term “combination therapy” as used herein is meant to refer to administration of two or more therapeutic agents in a sequential manner, that is, wherein each therapeutic agent is administered at a different time, as well as administration of two or more therapeutic agents in a simultaneous or substantially simultaneous manner. In some embodiments substantially simultaneously refers to administration of a second agent within 120, 90, 60 minutes or less from having been administered the first agent.

As used herein, the term “electroporation,” “electro-permeabilization,” or “electro-kinetic enhancement” (“EP”), are used interchangeably and are meant to refer to the use of a transmembrane electric field pulse to induce microscopic pathways (pores) in a bio-membrane; their presence allows biomolecules such as plasmids, oligonucleotides, siRNA, drugs, ions, and/or water to pass from one side of the cellular membrane to the other.

By “fragment” is meant a portion of a polypeptide or nucleic acid molecule, such as but not limiting to a truncation mutant. This portion contains, preferably, at least about 5%, 10%, 20%, 30%, 40%, 50%, 60%, 70%, 80%, 90%, 95% of the entire length of the reference nucleic acid molecule or polypeptide. A fragment may contain about 5, about 10, about 20, about 30, about 40, about 50, about 60, about 70, about 80, about 90, about 100, about 200, about 300, about 400, about 500, about 600, about 700, about 800, about 900, or about 1000 or more nucleotides or amino acids of a nucleotide or amino acid sequence, respectively.

The term “functional fragment” means any portion or fragment of a polypeptide or nucleic acid sequence from which the respective full-length polypeptide or nucleic acid relates that is of a sufficient length and has a sufficient structure to confer a biological affect that is similar or substantially similar to the full-length polypeptide or nucleic acid upon which the fragment is based. In some embodiments, a functional fragment is a portion of a full-length or wild-type nucleic acid sequence that encodes any one of the nucleic acid sequences disclosed herein, and said portion encodes a polypeptide of a certain length and/or structure that is less than full-length but encodes a domain that still biologically functional as compared to the full-length or wild-type protein. In some embodiments, the functional fragment may have a reduced biological activity, about equivalent biological activity, or an enhanced biological activity as compared to the wild-type or full-length polypeptide sequence upon which the fragment is based. In some embodiments, the functional fragment is derived from the sequence of an organism, such as a human. In such embodiments, the functional fragment may retain about 99%, about 98%, about 97%, about 96%, about 95%, about 94%, about 93%, about 92%, about 91%, or about 90% sequence identity to the wild-type or given sequence upon which the sequence is derived. In some embodiments, the functional fragment may retain about 85%, about 80%, about 75%, about 70%, about 65%, or about 60% sequence homology to the wild-type sequence upon which the sequence is derived.

As used herein, the term “genetic construct” is meant to refer to the DNA or RNA molecules that comprise a nucleotide sequence that encodes protein. The coding sequence includes initiation and termination signals operably linked to regulatory elements including a promoter and polyadenylation signal capable of directing expression in the cells of the individual to whom the nucleic acid molecule is administered.

The term “host cell” as used herein is meant to refer to a cell that can be used to express a nucleic acid, e.g., a nucleic acid of the disclosure. The host cell can be, but is not limited to, a eukaryotic cell, a bacterial cell, an insect cell, or a human cell. Suitable eukaryotic cells include, but are not limited to, Vero cells, HeLa cells, COS cells, CHO cells, HEK293 cells, BHK cells and MDCKII cells. Suitable insect cells include, but are not limited to, Sf9 cells. The phrase “recombinant host cell” can be used to denote a host cell that has been transformed or transfected with a nucleic acid to be expressed. A host cell also can be a cell that comprises the nucleic acid but does not express it at a desired level unless a regulatory sequence is introduced into the host cell such that it becomes operably linked with the nucleic acid. It is understood that the term host cell refers not only to the particular subject cell but also to the progeny or potential progeny of such a cell. Because certain modifications may occur in succeeding generations due to, e.g., mutation or environmental influence, such progeny may not, in fact, be identical to the parent cell, but are still included within the scope of the term as used herein.

The term “hybridize” as used herein is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

The term “immune checkpoint” as used herein is meant to refer to inhibitory pathways that slow down or stop immune reactions and prevent excessive tissue damage from uncontrolled activity of immune cells.

The term “immune response” is used herein is meant to refer to the activation of a host's immune system, e.g., that of a mammal, in response to the introduction of nucleic acid molecules comprising a nucleotide sequence encoding neoantigens a described herein.

The term “isolated” as used herein means that the polynucleotide or polypeptide or fragment, variant, or derivative thereof has been essentially removed from other biological materials with which it is naturally associated, or essentially free from other biological materials derived, e.g., from a recombinant host cell that has been genetically engineered to express the polypeptide of the disclosure.

The terms “in isolation” mean that, for purposes of this disclosure, the nucleic acid may not be the species listed. In other words, the nucleic acid may incorporate the mutations above in combination with one or more other mutations listed or not listed, but the nucleic acid may not be defined as the single species containing the nucleic acid mutations listed.

The term “ligand” as used herein is meant to refer to a molecule which has a structure complementary to that of a receptor and is capable of forming a complex with this receptor. A ligand is to be understood as meaning in particular a peptide or peptide fragment which has a suitable length and suitable binding motives in its amino acid sequence, so that the peptide or peptide fragment is capable of forming a complex with proteins of MHC class I or MHC class II.

The terms “MHC molecules”, “MHC proteins” or “HLA proteins” as used herein are meant to refer to proteins capable of binding peptides resulting from the proteolytic cleavage of protein antigens and representing potential T-cell epitopes, transporting them to the cell surface and presenting them there to specific cells, in particular cytotoxic T-lymphocytes or T-helper cells. The major histocompatibility complex in the genome comprises the genetic region whose gene products expressed on the cell surface are important for binding and presenting endogenous and/or foreign antigens and thus for regulating immunological processes. The major histocompatibility complex is classified into two gene groups coding for different proteins, namely molecules of MHC class I and molecules of MHC class II. The molecules of the two MHC classes are specialized for different antigen sources. The molecules of MHC class I present endogenously synthesized antigens, for example viral proteins and tumor antigens. The molecules of MHC class II present protein antigens originating from exogenous sources, for example bacterial products. The cellular biology and the expression patterns of the two MHC classes are adapted to these different roles.

In some embodiments, MHC molecules of class I comprise a heavy chain and a light chain and are capable of binding a peptide of about 8 to 11 amino acids, but usually 9 or 10 amino acids, if this peptide has suitable binding motifs, and presenting it to cytotoxic T-lymphocytes. The peptide bound by the MHC molecules of class I originates from an endogenous protein antigen. The heavy chain of the MHC molecules of class I is preferably an HLA-A, HLA-B or HLA-C monomer, and the light chain is β-2-microglobulin.

In some embodiments, MHC molecules of class II comprise an α-chain and a β-chain and are capable of binding a peptide of about 15 to 24 amino acids if this peptide has suitable binding motifs, and presenting it to T-helper cells. The peptide bound by the MHC molecules of class II usually originates from an extracellular of exogenous protein antigen. The α-chain and the β-chain are in particular HLA-DR, HLA-DQ and HLA-DP monomers.

The term “neoantigen” as used herein refers to a class of tumor antigens which arises from tumor-specific mutations in expressed protein of a subject. In some embodiments, the neoantigen is derived directly from a tumor of a subject. This is as opposed to a known tumor associated antigen which may be a consensus sequence known to elicit an immune response against a cell expressing the tumor antigen but not necessarily expressed by a tumor derived from the subject.

The term “neoantigen mutation” as used herein refers to a mutation that is predicted to encode a neoantigenic peptide.

The term “pharmaceutically acceptable” as used herein refers to approved or approvable by a regulatory agency of the Federal or a state government or listed in the U.S. Pharmacopeia or other generally recognized pharmacopeia for use in animals, including humans.

The term “pharmaceutically acceptable excipient, carrier or diluent” as used herein is meant to refer to an excipient, carrier or diluent that can be administered to a subject, together with an agent, and which does not destroy the pharmacological activity thereof and is nontoxic when administered in doses sufficient to deliver a therapeutic amount of the agent.

The term “pharmaceutically acceptable salt” of tumor specific neoantigens as used herein may be an acid or base salt that is generally considered in the art to be suitable for use in contact with the tissues of human beings or animals without excessive toxicity, irritation, allergic response, or other problem or complication. Such salts include mineral and organic acid salts of basic residues such as amines, as well as alkali or organic salts of acidic residues such as carboxylic acids. Specific pharmaceutical salts include, but are not limited to, salts of acids such as hydrochloric, phosphoric, hydrobromic, malic, glycolic, fumaric, sulfuric, sulfamic, suifanilic, formic, toluenesulfonie, methanesulfonic, benzene sulfonic, ethane disulfonic, 2-hydroxyethyl sulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenyiacetic, alkanoic such as acetic, HOOC—(CH2) n-COOH where n is from about 0 to about 4, and the like. Similarly, pharmaceutically acceptable cations include, but are not limited to sodium, potassium, calcium, aluminum, lithium and ammonium. Those of ordinary skill in the art will recognize from this disclosure and the knowledge in the art that further pharmaceutically acceptable salts for the pooled tumor specific neoantigens provided herein, including those listed by Remington's Pharmaceutical Sciences, 17th ed., Mack Publishing Company, Easton, PA, p. 1418 (1985). In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in an appropriate solvent.

As used herein, the terms “prevent,” “preventing,” “prevention,” “prophylactic treatment,” and the like, are meant to refer to reducing the probability of developing a disease or condition in a subject, who does not have, but is at risk of or susceptible to developing a disease or condition.

As used herein, the term “purified” means that the polynucleotide or polypeptide or fragment, variant, or derivative thereof is substantially free of other biological material with which it is naturally associated, or free from other biological materials derived, e.g., from a recombinant host cell that has been genetically engineered to express the polypeptide. That is, e.g., a purified polypeptide is a polypeptide that is at least from about 70 to about 100% pure, i.e., the polypeptide is present in a composition wherein the polypeptide constitutes from about 70 to about 100% by weight of the total composition. In some embodiments, the purified polypeptide is from about 75% to about 99% by weight pure, from about 80% to about 99% by weight pure, from about 90 to about 99% by weight pure, or from about 95% to about 99% by weight pure.

As used herein, the terms “subject,” “individual,” “host,” and “patient,” are used interchangeably herein and refer to any mammalian subject for whom diagnosis, treatment, or therapy is desired, particularly humans. The methods described herein are applicable to both human therapy and veterinary applications. In some embodiments, the subject is a mammal, and in other embodiments the subject is a human.

As used herein, “patient in need thereof” or “subject in need thereof” refers to a living organism suffering from or prone to a disease or condition that can be treated by administration of at least one composition, vaccine or pharmaceutical composition disclosed herein, including, for example, a vaccine comprising a nucleic acid sequence encoding a neoantigens, such as a nucleic acid sequence that encodes a beta-catenin antigen according to the methods described herein. A “patient in need thereof” or “subject in need” may also refer to a living organism that is receiving a neoantigen DNA vaccine (or pharmaceutical composition comprising a neoantigen DNA vaccine), or has received a neoantigen DNA vaccine (or pharmaceutical composition comprising a neoantigen DNA vaccine); or has a tumor or cancer. Non-limiting examples include humans, other mammals, such as bovines, rats, mice, dogs, monkeys, goat, sheep, cows, deer, and other non-mammalian animals. In embodiments, a patient in need thereof or subject in need thereof is human. In some embodiments, the subject in need thereof is a human patient that is suspected of having cancer or has been diagnosed with cancer and exhibits.

In some embodiments, the patient in need thereof or subject in need thereof has a cancer characterized by dysfunction of WNT or an abnormality in the WNT pathway. In some embodiments, the patient in need thereof or subject in need thereof has a cancer characterized by a dysfunction in WNT signaling. In some embodiments, the patient in need thereof or subject in need thereof has a cancer characterized by cells that exhibit axin destabilization and/or TNKS pathway dysfunction. In some embodiments, the patient in need thereof or subject in need thereof has a cancer that is characterized by one or more mutations in one or more WNT pathway signaling molecules. In some embodiments, the patient in need thereof or subject in need thereof has cancer that is characterized by one or more mutations in WNT, CTNNB1, AXIN1, AXIN2, APC, CK1, and/or GSK3B.

The term “T-cell epitope” as used herein is meant to refer to a peptide sequence which can be bound by the MHC molecules of class I or II in the form of a peptide-presenting MHC molecule or MHC complex and then, in this form, be recognized and bound by cytotoxic T-lymphocytes or T-helper cells, respectively.

The term “therapeutic effect” as used herein is meant to refer to some extent of relief of one or more of the symptoms of a disorder (e.g., a neoplasia or tumor) or its associated pathology. A “therapeutically effective amount” as used herein is meant to refer to an amount of an agent which is effective, upon single or multiple dose administration to the cell or subject, in prolonging the survivability of the patient with such a disorder, reducing one or more signs or symptoms of the disorder, preventing or delaying, and the like beyond that expected in the absence of such treatment. A “therapeutically effective amount” is intended to qualify the amount required to achieve a therapeutic effect. A physician or veterinarian having ordinary skill in the art can readily determine and prescribe the “therapeutically effective amount” (e.g., ED50) of the pharmaceutical composition required. For example, the physician or veterinarian could start doses of the compounds employed in a pharmaceutical composition at levels lower than that required in order to achieve the desired therapeutic effect and gradually increase the dosage until the desired effect is achieved.

In some embodiments, the therapeutically effective amount is an amount which results in the prevention or amelioration of or a decrease in the symptoms associated with a disease or disorder, i.e., a cancer, associated with Wnt signaling. The disclosed compound(s) can be administered to the subject either prior to or after the onset of a Wnt signaling-related disorder.

In some embodiments, the widest diameter of the tumor shrinks by about 2%, by about 4%, by about 6%, by about 8%, by about 10%, by about 15%, by about 20%, by about 25%, by about 30%, by about 35%, by about 40%, by about 45%, by about 50%, by about 60%, by about 70%, by about 80%, by about 90% or by about 100% as compared to the widest tumor diameter of the solid tumor before treatment.

In some embodiments, the size of a tumor is measured by Response Evaluation Criteria in Solid Tumors (“RECIST”) in which the longest diameter of the solid tumor as measured by radiological imaging, such as MRI or CT, is used as a proxy for tumor size. In some embodiments, the therapeutically effective amount is an amount effective to shrink a solid tumor by about 2% in size as measured by RECIST as compared to its size measured by RECIST before treatment, by about 4% in size, by about 6% in size, by about 8% in size, by about 10% in size, by about 15% in size, by about 20% in size, by about 25% in size, by about 30% in size, by about 35% in size, by about 40% in size, by about 45% in size, by about 50% in size, by about 60% in size, by about 70% in size, by about 80% in size, by about 90% in size or by about 100% in size as measured by RECIST as compared to the size of the solid tumor as measured by RECIST before treatment.

In some embodiments, the treatment results in a reduction of greater than 2% in size as measured by RECIST as compared to its size measured by RECIST before treatment, by greater than about 5% reduction in size, greater than about 10% reduction in size, greater than about 15% reduction in size, greater than about 20% reduction in size, greater than about 25% reduction in size, greater than about 30% reduction in size, greater than about 35% reduction in size, greater than about 40% reduction in size, greater than about 45% reduction in size, greater than about 50% reduction in size, greater than about 60% reduction in size, greater than about 70% reduction in size, greater than about 90% reduction in size, greater than about 90% reduction in size or about 100% reduction in size as measured by RECIST as compared to the size of the solid tumor as measured by RECIST before treatment. A 100% reduction in size is designated as a complete clinical response (CR).

In some embodiments, the therapeutically effective amount is an amount effective to shrink a solid tumor by about 2% in total mass as compared to its mass or estimated mass before treatment, by about 4% in total mass, by about 6% in total mass, by about 8% in total mass, by about 10% in total mass, by about 15% in total mass, by about 20% in total mass, by about 25% in total mass, by about 30% in total mass, by about 35% in total mass, by about 40% in total mass, by about 45% in total mass, or by about 50% in total mass as compared to the total mass of the solid tumor before the treatment.

The terms “treat,” “treated,” “treating,” “treatment,” and the like as used herein are meant to refer to reducing or ameliorating a disorder and/or symptoms associated therewith (e.g., a cancer or tumor). “Treating” may refer to administration of the neoantigen vaccines described herein to a subject after the onset, or suspected onset, of a cancer. “Treating” includes the concepts of “alleviating”, which refers to lessening the frequency of occurrence or recurrence, or the severity, of any symptoms or other ill effects related to a cancer and/or the side effects associated with cancer therapy. The term “treating” also encompasses the concept of “managing” which refers to reducing the severity of a particular disease or disorder in a patient or delaying its recurrence, e.g., lengthening the period of remission in a patient who had suffered from the disorder. It is appreciated that, although not precluded, treating a disorder or condition does not require that the disorder, condition, or symptoms associated therewith be completely eliminated.

As used herein, the term “treating cancer” is not intended to be an absolute term. In some aspects, the compositions and methods of the disclosure seek to reduce the size of a tumor or number of cancer cells, cause a cancer to go into remission, or prevent growth in size or cell number of cancer cells in a subject in need of treatment. In some circumstances, treatment with the disclosed compositions leads to an improved prognosis and/or extended life expectancy.

The terms “prophylaxis” or “prevention” means impeding the onset or recurrence of a disorder or one or more symptoms associated with a disorder.

For any therapeutic agent described herein the therapeutically effective amount may be initially determined from preliminary in vitro studies and/or animal models. A therapeutically effective dose may also be determined from human data. The applied dose may be adjusted based on the relative bioavailability and potency of the administered agent. Adjusting the dose to achieve maximal efficacy based on the methods described above and other well-known methods is within the capabilities of the ordinarily skilled artisan. General principles for determining therapeutic effectiveness, which may be found in Chapter 1 of Goodman and Gilman's The Pharmacological Basis of Therapeutics, 10th Edition, McGraw-Hill (New York) (2001), incorporated herein by reference, are summarized below.

Pharmacokinetic principles provide a basis for modifying a dosage regimen to obtain a desired degree of therapeutic efficacy with a minimum of unacceptable adverse effects. In situations where the drug's plasma concentration can be measured and related to the therapeutic window, additional guidance for dosage modification can be obtained.

Drug products are considered to be pharmaceutical equivalents if they contain the same active ingredients and are identical in strength or concentration, dosage form, and route of administration. Two pharmaceutically equivalent drug products are considered to be bioequivalent when the rates and extents of bioavailability of the active ingredient in the two products are not significantly different under suitable test conditions.

The terms “polynucleotide,” “oligonucleotide” and “nucleic acid” are used interchangeably throughout and include DNA molecules (e.g., cDNA or genomic DNA), RNA molecules (e.g., mRNA), analogs of the DNA or RNA generated using nucleotide analogs (e.g., peptide nucleic acids and non-naturally occurring nucleotide analogs), and hybrids thereof. The nucleic acid molecule can be single-stranded or double-stranded. In some embodiments, the nucleic acid molecules of the disclosure comprise a contiguous open reading frame encoding an antibody, or a fragment thereof, as described herein. “Nucleic acid” or “oligonucleotide” or “polynucleotide” as used herein may mean at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions. Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine Nucleic acids may be obtained by chemical synthesis methods or by recombinant methods.

A nucleic acid will generally contain phosphodiester bonds, although, in some embodiments, nucleic acid analogs may be included that may have at least one different linkage, e.g., phosphoramidate, phosphorothioate, phosphorodithioate, or O-methylphosphoroamidite linkages and peptide nucleic acid backbones and linkages. Other analog nucleic acids include those with positive backbones; non-ionic backbones, and non-ribose backbones, including those described in U.S. Pat. Nos. 5,235,033 and 5,034,506, which are incorporated by reference in their entireties. Nucleic acids containing one or more non-naturally occurring or modified nucleotides are also included within one definition of nucleic acids. The modified nucleotide analog may be located for example at the 5′-end and/or the 3′-end of the nucleic acid molecule. Representative examples of nucleotide analogs may be selected from sugar- or backbone-modified ribonucleotides. It should be noted, however, that also nucleobase-modified ribonucleotides, i.e. ribonucleotides, containing a non-naturally occurring nucleobase instead of a naturally occurring nucleobase such as uridines or cytidines modified at the 5-position, e.g. 5-(2-amino) propyl uridine, 5-bromo uridine; adenosines and guanosines modified at the 8-position, e.g. 8-bromo guanosine; deaza nucleotides, e.g. 7-deaza-adenosine; O- and N-alkylated nucleotides, e.g. N6-methyl adenosine are suitable. The 2′-OH-group may be replaced by a group selected from H, OR, R, halo, SH, SR, NH.sub.2, NHR, N.sub.2 or CN, wherein R is C.sub.1-C.sub.6 alkyl, alkenyl or alkynyl and halo is F, Cl, Br or I. Modified nucleotides also include nucleotides conjugated with cholesterol through, e.g., a hydroxyprolinol linkage as described in Krutzfeldt et al., Nature (Oct. 30, 2005), Soutschek et al., Nature 432:173-178 (2004), and U.S. Patent Publication No. 20050107325, which are incorporated herein by reference in their entireties. Modified nucleotides and nucleic acids may also include locked nucleic acids (LNA), as described in US20020115080, which is incorporated herein by reference. Additional modified nucleotides and nucleic acids are described in U.S. Patent Publication No. 20050182005, which is incorporated herein by reference in its entirety. Modifications of the ribose-phosphate backbone may be done for a variety of reasons, e.g., to increase the stability and half-life of such molecules in physiological environments, to enhance diffusion across cell membranes, or as probes on a biochip. Mixtures of naturally occurring nucleic acids and analogs may be made; alternatively, mixtures of different nucleic acid analogs, and mixtures of naturally occurring nucleic acids and analogs may be made. In some embodiments, the nucleotide sequence encoding one or more antigens is free of modified nucleotide analogs. In some embodiments, the nucleotide sequence encoding one or more antigens comprises from about 1 to about 20 nucleic acid modifications. In some embodiments, the nucleotide sequence encoding one or more antigens comprises from about 1 to about 50 nucleic acid modifications. In some embodiments, the nucleotide sequence encoding one or more antigens independently comprise from about 1 to about 100 nucleic acid modifications.

As used herein, the term “nucleic acid molecule” comprises one or more nucleotide sequences that encode one or more proteins. In some embodiments, a nucleic acid molecule comprises initiation and termination signals operably linked to regulatory elements including a promoter and polyadenylation signal capable of directing expression in the cells of the individual to whom the nucleic acid molecule is administered. In some embodiments, the nucleic acid molecule also is a plasmid comprising one or more nucleotide sequences that encode one or a plurality of neoantigens. In some embodiments, the disclosure relates to a pharmaceutical composition comprising a first, second, third or more nucleic acid molecules, each of which encoding one or a plurality of neoantigens and at least one of each plasmid comprising one or more of the Formulae disclosed herein.

The terms “polypeptide”, “peptide” and “protein” are used interchangeably herein to refer to polymers of amino acids of any length. The polymer may be linear or branched, it may comprise modified amino acids, and it may be interrupted by non-natural amino acids or chemical groups that are not amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics.

As used herein, “conservative” amino acid substitutions may be defined as set out in Tables A, B, or C below. The vaccines, compositions, pharmaceutical compositions and method may comprise nucleic acid sequences comprising one or more conservative substitutions. In some embodiments, the vaccines, compositions, pharmaceutical compositions and methods comprise nucleic acid sequences that retain from about 70% sequence identity to about 99% sequences identity to the sequence identification numbers disclosed herein but comprise one or more conservative substitutions. Conservative substitutions of the present disclosure include those wherein conservative substitutions (from either nucleic acid or amino acid sequences) have been introduced by modification of polynucleotides encoding polypeptides. Amino acids can be classified according to physical properties and contribution to secondary and tertiary protein structure. A conservative substitution is recognized in the art as a substitution of one amino acid for another amino acid that has similar properties. In some embodiments, the conservative substitution is recognized in the art as a substitution of one nucleic acid for another nucleic acid that has similar properties, or, when encoded, has similar binding affinities to its target. In some embodiments, the target is a cell expressing β-catenin. Exemplary conservative substitutions are set out in Table A.

TABLE A
Conservative Substitutions I

| | Side Chain Characteristics | Amino Acid |
|---|---|---|
| Aliphatic | Non-polar | G A P I L V F |
| | Polar-uncharged | C S T M N Q |
| | Polar-charged | D E K R |
| Aromatic | | H F W Y |
| Other | | N Q D E |

Alternately, conservative amino acids can be grouped as described in Lehninger, (Biochemistry, Second Edition; Worth Publishers, Inc. NY, N.Y. (1975), pp. 71-77) as set forth in Table B.

TABLE B
Conservative Substitutions II

| Side Chain Characteristic | Amino Acid |
|---|---|
| Non-polar (hydrophobic) | |
| Aliphatic: | A L I V P |
| Aromatic: | F W Y |
| Sulfur-containing: | M |
| Borderline: | G Y |
| Uncharged-polar | |
| Hydroxyl: | S T Y |
| Amides: | N Q |
| Sulfhydryl: | C |
| Borderline: | G Y |
| Positively Charged (Basic): | K R H |
| Negatively Charged (Acidic): | D E |

Alternately, exemplary conservative substitutions are set out in Table C.

TABLE C
Conservative Substitutions III

| Original Residue | Exemplary Substitution |
|---|---|
| Ala (A) | Val Leu Ile Met |
| Arg (R) | Lys His |
| Asn (N) | Gln |
| Asp (D) | Glu |
| Cys (C) | Ser Thr |
| Gln (Q) | Asn |
| Glu (E) | Asp |
| Gly (G) | Ala Val Leu Pro |
| His (H) | Lys Arg |
| Ile (I) | Leu Val Met Ala Phe |
| Leu (L) | Ile Val Met Ala Phe |
| Lys (K) | Arg His |
| Met (M) | Leu Ile Val Ala |
| Phe (F) | Trp Tyr Ile |
| Pro (P) | Gly Ala Val Leu Ile |
| Ser (S) | Thr |
| Thr (T) | Ser |
| Trp (W) | Tyr Phe Ile |
| Tyr (Y) | Trp Phe Thr Ser |
| Val (V) | Ile Leu Met Ala |

It should be understood that the inhibitors described herein are intended to include nucleic acids and, where the inhibitors include polypeptide, polypeptides bearing one or more insertions, deletions, or substitutions, or any combination thereof, of amino acid residues as well as modifications other than insertions, deletions, or substitutions of amino acid residues.

As used herein, “more than one” or “two or more” of the aforementioned amino acid substitutions means 2, 3, 4, 5, 6, 7, 8, 9, 10 or more of the recited amino acid or nucleic acid substitutions. In some embodiments, “more than one” means 2, 3, 4, or 5 of the recited amino acid substitutions or nucleic acid substitutions. In some embodiments, “more than one” means 2, 3, 4 or more of the recited amino acid substitutions or nucleic acid substitutions. In some embodiments, “more than one” means 2, 3 or 4 of the recited amino acid substitutions or nucleic acid substitutions. In some embodiments, “more than one” means 2 or more of the recited amino acid substitutions or nucleic acid substitutions. In some embodiments, “more than one” means 2 of the recited amino acid substitutions or nucleic acid substitutions.

The “percent identity” or “percent homology” of two polynucleotide or two polypeptide sequences is determined by comparing the sequences using the GAP computer program (a part of the GCG Wisconsin Package, version 10.3 (Accelrys, San Diego, Calif.)) using its default parameters. “Identical” or “identity” as used herein in the context of two or more nucleic acids or amino acid sequences, may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0. Briefly, the BLAST algorithm, which stands for Basic Local Alignment Search Tool is suitable for determining sequence similarity. Software for performing BLAST analyses is publicly available through the National Center for Biotechnology Information (http://www.ncbi.nlm.nih.gov). This algorithm involves first identifying high scoring sequence pair (HSPs) by identifying short words of length within a query sequence that either match or satisfy some positive-valued threshold score T when aligned with a word of the same length in a database sequence. T is referred to as the neighborhood word score threshold (Altschul et al., 1997). These initial neighborhood word hits act as seeds for initiating searches to find HSPs containing them. The word hits are extended in both directions along each sequence for as far as the cumulative alignment score can be increased. Extension for the word hits in each direction are halted when: 1) the cumulative alignment score falls off by the quantity X from its maximum achieved value; 2) the cumulative score goes to zero or below, due to the accumulation of one or more negative-scoring residue alignments; or 3) the end of either sequence is reached. The Blast algorithm parameters W, T and X determine the sensitivity and speed of the alignment. The Blast program uses as defaults a word length (W) of 11, the BLOSUM62 scoring matrix (see Henikoff et al., Proc. Natl. Acad. Sci. USA, 1992, 89, 10915-10919, which is incorporated herein by reference in its entirety) alignments (B) of 50, expectation (E) of 10, M=5, N=4, and a comparison of both strands. The BLAST algorithm (Karlin et al., Proc. Natl. Acad. Sci. USA, 1993, 90, 5873-5787, which is incorporated herein by reference in its entirety) and Gapped BLAST perform a statistical analysis of the similarity between two sequences. One measure of similarity provided by the BLAST algorithm is the smallest sum probability (P(N)), which provides an indication of the probability by which a match between two nucleotide sequences would occur by chance. For example, a nucleic acid is considered similar to another if the smallest sum probability in comparison of the test nucleic acid to the other nucleic acid is less than about 1, less than about 0.1, less than about 0.01, and less than about 0.001.

Two single-stranded polynucleotides are “the complement” of each other if their sequences can be aligned in an anti-parallel orientation such that every nucleotide in one polynucleotide is opposite its complementary nucleotide in the other polynucleotide, without the introduction of gaps, and without unpaired nucleotides at the 5′ or the 3′ end of either sequence. A polynucleotide is “complementary” to another polynucleotide if the two polynucleotides can hybridize to one another under moderately stringent conditions. Thus, a polynucleotide can be complementary to another polynucleotide without being its complement.

The phrase “stringent hybridization conditions” or “stringent conditions” as used herein is meant to refer to conditions under which a nucleic acid molecule will hybridize another nucleic acid molecule, but to no other sequences. Stringent conditions are sequence-dependent and will be different in different circumstances. Longer sequences hybridize specifically at higher temperatures. Generally, stringent conditions are selected to be about 5 °C lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present in excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30 °C for short probes, primers or oligonucleotides (e.g. 10 to 50 nucleotides) and at least about 600C for longer probes, primers or oligonucleotides. Stringent conditions may also be achieved with the addition of destabilizing agents, such as formamide.

By “substantially identical” is meant nucleic acid molecule (or polypeptide) exhibiting at least 50% identity to a reference amino acid sequence (for example, any one of the amino acid sequences described herein) or nucleic acid sequence (for example, any one of the nucleic acid sequences described herein). Preferably, such a sequence is at least about 60%, about 80% or about 85%, and about 90%, about 95% or about 99% identical at the amino acid level or nucleic acid to the sequence used for comparison.

A nucleotide sequence is “operably linked” to a regulatory sequence if the regulatory sequence affects the expression (e.g., the level, timing, or location of expression) of the nucleotide sequence. A “regulatory sequence” is a nucleic acid that affects the expression (e.g., the level, timing, or location of expression) of a nucleic acid to which it is operably linked. The regulatory sequence can, for example, exert its effects directly on the regulated nucleic acid, or through the action of one or more other molecules (e.g., polypeptides that bind to the regulatory sequence and/or the nucleic acid). Examples of regulatory sequences include promoters, enhancers and other expression control elements (e.g., polyadenylation signals). Further examples of regulatory sequences are described in, for example, Goeddel, 1990, Gene Expression Technology: Methods in Enzymology 185, Academic Press, San Diego, Calif. and Baron et al., 1995, Nucleic Acids Res. 23:3605-06.

As used herein, the term “sample” refers generally to a limited quantity of something which is intended to be similar to and represent a larger amount of that something. In the present disclosure, a sample is a collection, swab, brushing, scraping, biopsy, removed tissue, or surgical resection that is to be testing for the absence, presence or grading of a tissue, which, in some cases is cancerous tissue or one or a plurality of cells. In some embodiments, samples are taken from a patient or subject that is believed to have a cancer, hyperplasia, pre-cancerous or comprise one or more tumor cells. In some embodiments, a sample believed to contain one or more malignant, cancerous or pre-cancerous cells are compared to a “control sample” that is known to be free of one or more malignant, cancerous or pre-cancerous cells. This disclosure contemplates using any one or a plurality of disclosed samples herein to identify, detect, sequence and/or quantify the amount of neoantigens (highly or minimally immunogenic) within a particular sample. In some embodiments, the methods relate to the step of exposing a swab, brushing or other sample from an environment to a set of reagents sufficient to isolate and/or sequence the DNA and RNA of one or a plurality of cells in the sample.

The disclosure relates to a composition comprising a vector. A “vector” is a nucleic acid molecule that can be used to introduce a nucleic acid sequence subcomponent linked to it into a cell. One type of vector is a “plasmid,” which refers to a linear or circular double stranded DNA molecule into which additional nucleic acid segments can be ligated. Another type of vector is a viral vector (e.g., replication defective retroviruses, adenoviruses and adeno-associated viruses), in the form of an RNA, DNA or hybrid RNA/DNA molecule comprising viral genome promoter sequences are operably linked to the expressible nucleotide sequence. In some embodiments, the expressible nucleotide sequence is introduced into the viral genome. Certain vectors are capable of autonomous replication in a host cell into which they are introduced (e.g., bacterial vectors comprising a bacterial origin of replication and episomal mammalian vectors). Other vectors (e.g., non-episomal mammalian vectors) are integrated into the genome of a host cell upon introduction into the host cell, and thereby are replicated along with the host genome. An “expression vector” is a type of vector that can direct the expression of a chosen polynucleotide. The disclosure relates to any one or plurality of vectors that comprise nucleic acid sequences encoding any one or plurality of amino acid sequence disclosed herein.

The term “vaccine” as used herein is meant to refer to a composition for generating immunity for the prophylaxis and/or treatment of diseases (e.g., cancer). Accordingly, vaccines are medicaments which comprise antigens and are intended to be used in humans or animals for generating specific defense and protective substance by vaccination. A “vaccine composition” or a “neoantigen vaccine composition” can include a pharmaceutically acceptable excipient, carrier or diluent.

The disclosure also relates to one or more amino acid sequences comprising any one or combination of antigen amino acid sequences disclosed herein. In some embodiments, the expressible nucleotide sequences of the disclosure encode one or more of the amino acid sequences. In some embodiments, compositions of the disclosure comprise chimeric or fusion proteins, in which one amino acid sequence that is an antigen is fused contiguously or non-contiguously to a second amino acid sequence. As used herein, a “chimeric protein” or “fusion protein” comprises all or part (preferably biologically active) of a polypeptide or compound of the disclosure operably linked to a heterologous amino acid sequence (i.e., an amino acid sequence other than the compound of the disclosure). Within the fusion protein, the term “operably linked” is intended to indicate that the polypeptide of the disclosure and the heterologous polypeptide are fused in frame to each other. The heterologous polypeptide can be fused to the N terminus or C terminus of any one or plurality of polypeptides of the disclosure. In some embodiments, the disclosure relates to a composition comprising an amino acid sequence comprising a first amino acid sequence that is an antigen fused to a second amino acid sequence that is protein tag, wherein the tag is a marker detectable after exposed to a stimulus or a marker that can associate with one or more amino acids on a solid support such that the marker facilitates isolation of the antigen.

One useful fusion protein is a GST fusion protein in which the polypeptide of the disclosure is fused to the C terminus of one or a plurality of GST sequences. Such fusion proteins can facilitate the purification of a recombinant polypeptide of the disclosure.

In some embodiments, the fusion protein contains a heterologous signal sequence at its N terminus. For example, the native signal sequence of a polypeptide of the disclosure can be removed and replaced with a signal sequence from another protein. For example, the gp67 secretory sequence of the baculovirus envelope protein can be used as a heterologous signal sequence (Current Protocols in Molecular Biology, Ausubel et al., eds., John Wiley & Sons, 1992). Other examples of eukaryotic heterologous signal sequences include the secretory sequences of melittin and human placental alkaline phosphatase (Stratagene; La Jolla, Calif.). In yet another example, useful prokaryotic heterologous signal sequences include the phoA secretory signal (Sambrook et al., supra) and the protein A secretory signal (Pharmacia Biotech; Piscataway, N.J.).

In some embodiments, the fusion protein is an immunoglobulin fusion protein in which all or part of a polypeptide of the disclosure is fused to sequences derived from a member of the immunoglobulin protein family. The immunoglobulin fusion proteins of the disclosure can be incorporated into pharmaceutical compositions and administered to a subject to inhibit an interaction between a ligand (soluble or membrane bound) and a protein on the surface of a cell (receptor), to thereby suppress signal transduction in vivo. The immunoglobulin fusion protein can be used to affect the bioavailability of a cognate ligand of a polypeptide of the disclosure. Inhibition of ligand/receptor interaction may be useful therapeutically, both for treating proliferative and differentiative disorders and for modulating (e.g., promoting or inhibiting) cell survival. Moreover, the immunoglobulin fusion proteins of the disclosure can be used as immunogens to produce antibodies directed against a polypeptide of the disclosure in a subject, to purify ligands and in screening assays to identify molecules which inhibit the interaction of receptors with ligands.

Chimeric and fusion proteins of the disclosure can be produced by standard recombinant DNA techniques. In some embodiments, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, e.g., Ausubel et al., supra). Moreover, many expression vectors are commercially available that already encode a fusion moiety (e.g., a GST polypeptide). A nucleic acid encoding a polypeptide of the disclosure can be cloned into such an expression vector such that the fusion moiety is linked in frame to the polypeptide of the disclosure.

### Compositions

The present disclosure relates to a step of identifying a plurality of mutations within a cancer/tumor (e.g., translocations, inversions, large and small deletions and insertions, missense mutations, splice site mutations, etc.). In particular, these mutations are present in the genome of cancer/tumor cells of a subject, but not in normal tissue from the subject. The disclosure relates to the innovative discovery that administering pharmaceutical compositions comprising the nucleic acid sequences that encode from about 1 to about 100 different amino acid sequences that represent a milieu of mutations in several different cancer cells where at least one or from about one to about five mutations is an amino acid sequence from a cancer with WNT pathway dysfunction. Such mutations are of particular interest if they lead to changes that result in a protein with an altered amino acid sequence that is unique to the patient's cancer/tumor (e.g., a neo-antigen). In some embodiments, the present disclosure relates to treating or preventing cancer in a subject in need of treatment, wherein the subject has a cancer characterized by a dysfunction in the WNT pathway.

A cancer having a WNT pathway dysfunction is a cancer comprising at least one mutation in at least one gene of a molecule active in the WNT pathway. In some embodiments, the mutation results in at least one change of the amino acid sequence of a Wnt pathway molecule. In some embodiments, the mutation is a substitution mutation, addition or deletion in any part of a Wnt pathway molecule. In some embodiments, the mutation is any mutation that causes non-expression or limited expression of a Wnt pathway molecule resulting in an altered biological phenotype because of the low, limitied or deficient expression. In some embodiments, the mutation results in overexpression of a Wnt pathway molecule. In some embodiments, the mutations results in aberrant regulation of expression of a Wnt pathway molecule. Wnt pathway molecules include, but are not limited to, WNT, CTNNB1 (or beta-catenin), AXIN1, AXIN2, APC, CK1 and GSK3B. Mutations present in a cancer can be determined by any method known in the art, including, but not limited to, DNA sequencing and assays that detect the presence or quantity of the biomarker. Abberrant regulation of expression of a WNT pathway molecule can be determined by any method known in the art, including, but not limited to, quantifying RNA and/or protein expression levels. Abberrant regulation of expression of a WNT pathway molecule means that it is expressed either more than about 10% above (overexpressed) or more than about 10% below (underexpresssed) expression levels in non-tumor cells. Tumor Mutational Burden or TMB can be measured as disclosed in N Engl J Med (Dec. 21, 2017; 377:2500-250), which is incorporated by reference in its entirety, in which mutuational burden is quantified by examining expression profiles of biopsied tumor tissue throughout 27 different cancer types.

In some embodiments, the cancer is characterized by a high tumor mutational burden (TMB). Tumor mutational burden is the calculated frequency of certain mutations within a tumor's genes. To be counted toward TMB, mutations must alter a protein expressed by the tumor. Each of these mutations results in a protein that is an antigen and can be recognized by and activate the immune system. Methods of determining a high mutational burden are known in the art and include, for example, next generation whole exome sequencing, which sequences all of the protein-coding genes within a tumor, and sequencing a gene panel, which provides the sequences of a targeted set of genes. Tumors having a high mutational burden have at least about 10 mutations per million base pairs of tumor DNA.

In some embodiments, the cancer is characterized by resistance to checkpoint inhibitors. Resistance (poor or low response) to checkpoint inhibitor therapy means that there is less than about 2% reduction in tumor mass, no reduction in tumor mass or tumor growth after treatment with checkpoint inhibitors.

In some embodiments, the compositions disclosed herein are used to treat a cancer selected from: adrenocortical cancer, adrenocortical carcinoma, bladder cancer, bladder urothelial carcinoma, breast cancer, cervical adenocarcinoma, cervical squamous cell carcinoma, cholangiocarcinoma, colorectal adenocarcinoma, colorectal cancer, diffuse glioma, endometrial cancer, endometrial carcinoma, esophageal squamous cell carcinoma, esophagogastric adenocarcinoma, gastroesophageal junction adenocarcinoma, glioblastoma, head and neck squamous cell carcinoma, hepatocellular carcinoma, invasive breast carcinoma, leukemia, mature B-cell neoplasms, melanoma, mesothelioma, miscellaneous neuroepithelial tumor, non-seminomatous germ cell tumor, non-small cell lung cancer, ocular melanoma, ovarian cancer, ovarian epithelial tumor, pancreatic adenocarcinoma, pancreatic ductal adenocarcinoma, pheochromocytoma, pleural mesothelioma, prostate adenocarcinoma, prostate cancer, renal clear cell carcinoma, renal non-clear cell carcinoma, sarcoma, seminoma, thymic cancer, thymic epithelial tumor, thyroid cancer, undifferentiated stomach adenocarcinoma or well-differentiated thyroid cancer.

The present disclosure features a nucleic acid molecule comprising a nucleic acid sequence comprising Formula I:

[(AEDn)-(linker)]n-[AEDn+1],
wherein the AED is an independently selectable antigen expression domain comprising an expressible nucleic acid sequence, wherein AEDn is referred to as antigen expression domain and wherein AEDn+1 is referred to as antigen expression domain 2; wherein the each linker is independently selectable from about 0 to about 300 natural or non-natural nucleic acids in length, wherein the antigen expression domain 1 is independently selectable from about 12 to about 15,000 nucleotides in length and encodes a tumor-specific epitope of the subject; wherein the antigen expression domain 2 is independently selectable from about 12 to about 15,000 nucleotides in length and encodes a second tumor-specific epitope; and wherein n is any positive integer from about 1 to about 500. In some embodiments, n is equal to at least 19 or more. In some embodiments, n is equal to from about 19 to about 59.

In some embodiments, each linker is independently selectable from about 0 to about 25, about 1 to about 25, about 2 to about 25, about 3 to about 25, about 4 to about 25, about 5 to about 25, about 6 to about 25, about 7 to about 25, about 8 to about 25, about 9 to about 25, about 10 to about 25, about 11 to about 25, about 12 to about 25, about 13 to about 25, about 14 to about 25, about 15 to about 25, about 16 to about 25, about 17 to about 25, about 18 to about 25, about 19 to about 25, about 20 to about 25, about 21 to about 25, about 22 to about 25, about 23 to about 25, about 24 to about 25 natural or non-natural nucleic acids in length. In some embodiments, each linker is about 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25 natural or non-natural nucleic acids in length. In some embodiments, each linker is independently selectable from a linker that is about 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25 natural or non-natural nucleic acids in length. In some embodiments, each linker is about 21 natural or non-natural nucleic acids in length.

In some embodiments, the length of each linker according to Formula I is different. For example, in some embodiments, the length of a first linker is about 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25 natural or non-natural nucleic acids in length, and the length of a second linker is about 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25 natural or non-natural nucleic acids in length, where the length of the first linker is different from the length of the second linker. Various configurations can be envisioned by the present disclosure, where Formula I comprises 1, 2, 3, 4, 5, 6, 7, 8, 9, 10 or more linkers wherein the linkers are of similar or different lengths. In some embodiments, there is only linker linker between each nucleic acid sequence encoding a tumor-specific antigen, and as such the 5′ and the 3′ terminal tumor-specific nucleic acid sequences are adjacent to only one linker each. In some embodiments, the 5′ terminal tumor-specific linker is 3′ from a Ig leader sequence, such as an IgE leader sequence. In some embodiments, the expressible nucleic acid sequence comprises from about 20 to about 60 tumor-specific antigens with one linker in between each nucleic acid sequence encoding a tumor-specific antigen and the expressible nucleic acid sequence comprises a nucleic acid sequence on the 5′ position of the nucleic acid sequence encoding a first tumor-specific antigen, such nucleic acid sequence encoding a leader, and wherein the entire expressible nucleic acid sequence is operably linked to a promoter and/or an enhancer sequence within a nucleic acid molecule.

In certain embodiments, two linkers can be used together, in a nucleotide sequence that encodes a fusion peptide. Accordingly, in some embodiments, the first linker is independently selectable from about 0 to about 25 natural or non-natural nucleic acids in length, about 0 to about 25, about 1 to about 25, about 2 to about 25, about 3 to about 25, about 4 to about 25, about 5 to about 25, about 6 to about 25, about 7 to about 25, about 8 to about 25, about 9 to about 25, about 10 to about 25, about 11 to about 25, about 12 to about 25, about 13 to about 25, about 14 to about 25, about 15 to about 25, about 16 to about 25, about 17 to about 25, about 18 to about 25, about 19 to about 25, about 20 to about 25, about 21 to about 25, about 22 to about 25, about 23 to about 25, about 24 to about 25 natural or non-natural nucleic acids in length. In some embodiments, the second linker is independently selectable from about 0 to about 25, about 1 to about 25, about 2 to about 25, about 3 to about 25, about 4 to about 25, about 5 to about 25, about 6 to about 25, about 7 to about 25, about 8 to about 25, about 9 to about 25, about 10 to about 25, about 11 to about 25, about 12 to about 25, about 13 to about 25, about 14 to about 25, about 15 to about 25, about 16 to about 25, about 17 to about 25, about 18 to about 25, about 19 to about 25, about 20 to about 25, about 21 to about 25, about 22 to about 25, about 23 to about 25, about 24 to about 25 natural or non-natural nucleic acids in length. In some embodiments, the first linker is independently selectable from a linker that is about 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25 natural or non-natural nucleic acids in length. In some embodiments, the second linker is independently selectable from a linker that is about 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25 natural or non-natural nucleic acids in length.

In certain embodiments, antigen expression domain 1 and antigen expression domain 2 comprise a nucleic acid sequence that encodes one or two epitopes of a particular tumor-specific neoantigen. In some embodiments, antigen expression domain 1 encodes a CD4 neoepitope. In some embodiments, antigen expression domain 1 encodes a CD8 neoepitope. In some embodiments, antigen expression domain 2 encodes a CD4 neoepitope. In some embodiments, antigen expression domain 2 encodes a CD8 neoepitope. In some embodiments, antigen domain 1 encodes a CD8 neoepitope and antigen expression domain 2 encodes a CD8 neoepitope. A CD4 neoepitope is an epitope that is recognized by CD4+ T cells. A CD8 neoepitope is an epitope that is recognized by CD8+ T cells.

The disclosures also relates to a nucleic acid sequence comprising a plurality of antigen expression domains encoding at least two neoantigens separated by one or a plurality of linkers. In some embodiments, the antigen expression domain encodes an amino acid sequence from about 3 to about 100 amino acids in length. In some embodiments, there is at least one linker encoding a linker from about 3 to about 25 amino acids in length. In some embodiment, the linker sequence separate each antigen expression domain. In some embodiments, the nucleic acid sequence comprises 1, 2, 3, 4, 5, 6, 7, 8, 9, or 10 or more linkers. In some embodiments, the nucleic acid sequence comprises from about 10 to about 70 linkers, from about 15 to about 70 linkers, from about 20 to about 65 linkers, from about 25 to about 65 linkers, from about 30 to about 60 linkers, from about 35 to about 60 linkers, from about 40 to about 60 linkers, from about 45 to about 60 linkers, from about 50 to about 60 linkers or from about 52 to about 58 linkers. In some embodiments, the nucleic acid sequence comprises 52 linkers, 53 linkers, 54 linkers, 55 linkers, 56 linkers, 57 linkers or 58 linkers. In some embodiments, the nucleic acid comprises 55 linkers.

In some embodiments, the nucleic acid sequence comprises 1, 2, 3, 4, 5, 6, 7, 8, 9, or 10 or more linkers, wherein at least one or more linkers comprise a furin linker. In some embodiments, the nucleic acid sequence comprises from about 10 to about 70 linkers, from about 15 to about 70 linkers, from about 20 to about 65 linkers, from about 25 to about 65 linkers, from about 30 to about 60 linkers, from about 35 to about 60 linkers, from about 40 to about 60 linkers, from about 45 to about 60 linkers, from about 50 to about 60 linkers or from about 52 to about 58 linkers, wherein at least one or more linkers comprise a furin linker. In some embodiments, the nucleic acid sequence comprises 52 linkers, 53 linkers, 54 linkers, 55 linkers, 56 linkers, 57 linkers or 58 linkers, wherein at least one or more linkers comprise a furin linker. In some embodiments, the nucleic acid comprises 55 linkers, wherein at least one or more linkers comprise a furin linker.

In some embodiments, the nucleic acid sequence comprises at least 2, 3, 4, 5, 6, 7, 8, 9, or 10 or more linker domains and the nucleic acid sequence comprises Formula I (a):

[(AEDn)-(linker)]n-(AEDn+1)]n
wherein each AED is independently selectable from any one or plurality of tumor associated antigens from a subject and wherein n is any positive integer from about 1 to about 50 and wherein each “linker” is a nucleic acid sequence encoding one or a plurality of amino acid cleavage sites. Each linker may be the same or independently selectable to comprise one or a plurality of the linkers disclosed herein. In some embodiments, the linker is a furin cleavage site from about 9 to about 105 nucleotides in length and encodes an amino acid sequence that is an amino acid cleavage site. In some embodiments, the nucleic acid sequence is a component of a nucleic acid molecule. In some compositions contemplated herein, the composition comprises 1, 2, 3, 4, 5, or more nucleic acid molecules each of which expressing any of the patterns or formulae of AEDs disclosed herein.

In some embodiments, the nucleic acid sequence comprises at least 2, 3, 4, 5, 6, 7, 8, 9, or 10 or more linker domains and the nucleic acid sequence comprises Formula III (a):

[(AEDn)-(linker)]n-(AEDn+1)-linkern+1-(AEDn+2)]n

wherein each AED is independently selectable from any one or plurality of tumor associated antigens from a subject and wherein n is any positive integer from about 20 to about 50 and wherein each “linker” is a nucleic acid sequence encoding one or a plurality of amino acid cleavage sites. Each linker may be the same or independently selectable to comprise one or a plurality of the linkers disclosed herein; and wherein each “-” represents a bond between each subunit. In some embodiments, the linker is a furin cleavage site from about 9 to about 105 nucleotides in length and encodes an amino acid sequence that is an amino acid cleavage site. In some embodiments, the nucleic acid sequence is a component of a nucleic acid molecule. In some embodiments, the Formula III (a) comprises a third linker bonded to the 3′ end of third AED sequence. In some embodiments, the last AED sequence in 5′ to 3′ orientation does not bond to a linker.

The disclosures also relates to a nucleic acid sequence comprising a coding region and a non-coding region, the coding region consisting of the Formula I (b):
[(AED¹)-(linker)-(AED²)-(linker)]ₙ.-[(AED³)]ₙ₊₁, wherein n is a positive integer from about 1 to about 30, wherein each “linker” encodes one or a plurality of amino acid cleavages sequences, and wherein the non-coding region comprises at least one regulatory sequence operably linked to one or more AEDs; and wherein, in the 5′ ot 3′ orientation, AED³ is the terminal antigen expression domain in a sequence of AEDs. In some embodiments, the regulatory sequence is any of the regulatory sequences depicted in the Figures or a functional fragment that comprises at least 70%, 75%, 80%, 85%, 90%, 91%, 92%, 93%, 94%, 95%, 96% 97%, 98% or 99% sequence identity to the regulatory sequence depicted in the Figures.

In some embodiments, the nucleic acid molecule or sequence of the disclosure comprises a plurality of antigen expression domains encoding at least two neoantigens separated by one or a plurality of linkers. In some embodiments, the antigen expression domain encodes an amino acid sequence from about 3 to about 100 amino acids in length. In some embodiments, there is at least one linker encoding a linker from about 3 to about 25 amino acids in length. In some embodiment, the linker sequence separate each antigen expression domain. In some embodiments, the nucleic acid sequence comprises 1, 2, 3, 4, 5, 6, 7, 8, 9, or 10 or more linkers. In some embodiments, the nucleic acid sequence comprises from about 10 to about 70 linkers, from about 15 to about 70 linkers, from about 20 to about 65 linkers, from about 25 and to about 65 linkers, from about 30 to about 60 linkers, from about 35 to about 60 linkers, from about 40 to about 60 linkers, from about 45 to about 60 linkers, from about 50 to about 60 linkers or from about 52 to about 58 linkers. In some embodiments, the nucleic acid sequence comprises 52 linkers, 53 linkers, 54 linkers, 55 linkers, 56 linkers, 57 linkers or 58 linkers, wherein each linker ir positioned between at least two antigen expression domains encoding a tumor-specific antigen. In some embodiments, the nucleic acid comprises 55 linkers. In some embodiments, the nucleic acid sequence comprises 1, 2, 3, 4, 5, 6, 7, 8, 9, or 10 or more linkers, at least one or more are comprise furin linkers.

In some embodiments, the nucleic acid sequence comprises at least 2, 3, 4, 5, 6, 7, 8, 9, or 10 or more linker domains and the nucleic acid sequence comprises Formula I (a): (AED1)-(linker)-(AED2)]n wherein each AED is independently selectable from any one or plurality of tumor associated antigens from a subject and wherein n is any positive integer from about 1 to about 60. In some embodiments, n is about In some embodiments, each “linker” is a nucleic acid sequence encoding one or a plurality of amino acid cleavage sites. Each linker may be the same or independently selectable to comprise one or a plurality of the linkers disclosed herein. In some embodiments, n is a whole integer value from about 20 to about 30.

In some embodiments, the antigen expression domain 1 and/or 2 is independently selectable from about 12 to about 15,000 nucleotides in length, about 50 to about 15,000 nucleotides in length, about 100 to about 15,000 nucleotides in length, about 500 to about 15,000 nucleotides in length, about 1,000 to about 15,000 nucleotides in length, about 5,000 to about 15,000 nucleotides in length, about 10,000 to about 15,000 nucleotides in length. In other embodiments, the antigen expression domain 1 is about 12, about 25, about 50, about 75, about 100, about 200, about 300, about 400, about 500, about 600, about 700, about 800, about 900, about 1,000, about 2,000, about 3,000, about 4,000, about 5,000, about 6,000, about 7,000, about 8,000, about 9,000, about 10,000, about 11,000, about 12,000, about 13,000, about 14,000, about 15,000 nucleotides in length. In some embodiments, the antigen expression domain 2 is independently selectable from about 12 to about 15,000 nucleotides in length, about 50 to about 15,000 nucleotides in length, about 100 to about 15,000 nucleotides in length, about 500 to about 15,000 nucleotides in length, about 1,000 to about 15,000 nucleotides in length, about 5,000 to about 15,000 nucleotides in length, about 10,000 to about 15,000 nucleotides in length. In some embodiments, the antigen expression domain 2 is about 12, about 25, about 50, about 75, about 100, about 200, about 300, about 400, about 500, about 600, about 700, about 800, about 900, about 1,000, about 2,000, about 3,000, about 4,000, about 5,000, about 6,000, about 7,000, about 8,000, about 9,000, about 10,000, about 11,000, about 12,000, about 13,000, about 14,000 about 15,000 nucleotides in length.

In some embodiments, the antigen expression domain 1 or the antigen expression domain 2 are independently selectable from about 20 to about 2,000 nucleotides in length. In some embodiments, the antigen expression domain 1 is about 20 to about 2,000 nucleotides in length, about 50 to about 2,000 nucleotides in length, about 100 to about 2,000 nucleotides in length, about 500 to about 2,000 nucleotides in length, about 1500 to about 2,000 nucleotides in length. In other embodiments, the antigen expression domain 1 is about 20, about 30, about 40, about 50, about 60, about 70, about 80, about 90, about 100, about 200, about 300, about 400, about 500, about 600, about 700, about 800, about 900, about 1000, about 1,100, about 1,200, about 1,300, about 1,400, about 1,500, about 1,600, about 1,700, about 1,800, about 1900, about 2000 nucleotides in length. In some embodiments, the antigen expression domain 2 is about 20 to about 2,000 nucleotides in length, about 50 to about 2,000 nucleotides in length, about 100 to about 2,000 nucleotides in length, about 500 to about 2,000 nucleotides in length, about 1500 to about 2,000 nucleotides in length. In other embodiments, the antigen expression domain 2 is about 20, about 30, about 40, about 50, about 60, about 70, about 80, about 90, about 100, about 200, about 300, about 400, about 500, about 600, about 700, about 800, about 900, about 1000, about 1,100, about 1,200, about 1,300, about 1,400, about 1,500, about 1,600, about 1,700, about 1,800, about 1900, about 2000 nucleotides in length.

In some embodiments, the antigen expression domain 1 and/or the antigen expression domain 2 are independently selectable from about 15 to about 150 nucleotides in length, for example about 15 to about 150 nucleotides in length, about 15 to about 125 nucleotides in length, about 15 to about 100, about 15 to about 90 nucleotides in length, about 15 to about 90 nucleotides in length, about 15 to about 80 nucleotides in length, about 15 to about 70 nucleotides in length, about 15 to about 60 nucleotides in length, about 15 to about 50 nucleotides in length, about 15 to about 40 nucleotides in length, about 15 to about 30 nucleotides in length, about 15 to about 20 nucleotides in length.

In some embodiments, the antigen expression domain 1 and/or antigen expression domain 2 is independently selectable from about 15 to about 100 nucleotides in length, for example about 3 to about 120 nucleotides in length, from about 15 to about 100, from about 15 to about 90 nucleotides in length, about 15 to about 90 nucleotides in length, about 15 to about 80 nucleotides in length, about 15 to about 70 nucleotides in length, about 15 to about 60 nucleotides in length, about 15 to about 50 nucleotides in length, about 15 to about 40 nucleotides in length, about 15 to about 30 nucleotides in length, about 15 to about 20 nucleotides in length.

In some embodiments, the antigen expression domain 1 and/or antigen expression domain 2 is independently selectable from about 15 to about 50 nucleotides in length, for example about 15 to about 50 nucleotides in length, about 15 to about 40 nucleotides in length, about 15 to about 30 nucleotides in length, about 15 to about 20 nucleotides in length.

In some embodiments, n is any positive integer from about 1 to about 500. In some embodiments, n is any positive integer from about 1 to about 500, from about 10 to about 500, from about 50 to about 500, from about 100 to about 500, from about 200 to about 500, from about 300 to about 500, from about 400 to about 500. In other embodiments, n is any positive integer of about 1, about 5, about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 205, about 210, about 215, about 220, about 225, about 230, about 235, about 240, about 245, about 250, about 255, about 260, about 265, about 270, about 275, about 280, about 285, about 290, about 295, about 300, about 305, about 310, about 315, about 120, about 325, about 330, about 335, about 340, about 345, about 350, about 355, about 360, about 365, about 370, about 375, about 380, about 385, about 390, about 395, about 400, about 405, about 410, about 415, about 420, about 425, about 430, about 435, about 440, about 445, about 450, about 455, about 460, about 465, about 470, about 475, about 480, about 485, about 490, about 495, about 500.

In some embodiments, n is a positive integer from about 5 to about 30, from about 5 to about 25, from about 5 to about 20, from about 5 to about 15, from about 5 to about 10.

In some embodiments, n is a positive integer from about 2 to about 100, from about 2 to about 90, from about 2 to about 80, from about 2 to about 70, from about 2 to about 60, from about 2 to about 50, from about 2 to about 40, from about 2 to about 30, from about 2 to about 20, from about 2 to about 10.

In some embodiments, n is a positive integer from about 2 to about 58, from about 3 to about 58, from about 4 to about 58, from about 5 to about 58, from about 6 to about 58, from about 7 to about 58, from about 8 to about 58, from about 9 to about 58, from about 10 to about 58, from about 11 to about 58, from about 12 to about 58, from about 13 to about 58, from about 14 to about 58, from about 15 to about 58, from about 16 to about 58, from about 17 to about 58, from about 18 to about 58, from about 19 to about 58, from about 20 to about 58, from about 21 to about 58, from about 22 to about 58, from about 23 to about 58, from about 24 to about 58, from about 25 to about 58, from about 26 to about 58, from about 27 to about 58, from about 28 to about 58, from about 29 to about 58, from about 30 to about 58, from about 31 to about 58, from about 32 to about 58, from about 33 to about 58, from about 34 to about 58, from about 35 to about 58, from about 36 to about 58, from about 37 to about 58, from about 38 to about 58, from about 39 to about 58, from about 40 to about 58, from about 41 to about 58, from about 42 to about 58, from about 43 to about 58, from about 44 to about 58, from about 45 to about 58, from about 46 to about 58, from about 47 to about 58, from about 48 to about 58, from about 49 to about 58, from about 50 to about 58, from about 51 to about 58, from about 52 to about 58, from about 53 to about 58, from about 54 to about 58, from about 55 to about 58, from about 56 to about 58, from about 57 to about 58.

In some embodiments, n is a positive integer from about 2 to about 29, from about 3 to about 29, from about 4 to about 29, from about 5 to about 29, from about 6 to about 58, from about 7 to about 29, from about 8 to about 29, from about 9 to about 29, from about 10 to about 29, from about 11 to about 29, from about 12 to about 29, from about 13 to about 29, from about 14 to about 29, from about 15 to about 29, from about 16 to about 29, from about 17 to about 29, from about 18 to about 29, from about 19 to about 29, from about 20 to about 29, from about 21 to about 29, from about 22 to about 29, from about 23 to about 29, from about 24 to about 29, from about 25 to about 29, from about 26 to about 29, from about 27 to about 29, from about 28 to about 29.

In some embodiments, n is any positive integer from about 30 to about 60. In some embodiments, n is any positive integer from about 35 to about 60. In some embodiments, n is any positive integer from about 40 to about 60. In some embodiments, n is any positive integer from about 45 to about 60. In some embodiments, n is any positive integer from about 50 to about 60. In some embodiments, n is 50. In some embodiments, n is 51. In some embodiments, n is 52. In some embodiments, n is 53. In some embodiments, n is 54. In some embodiments, n is 55. In some embodiments, n is 56. In some embodiments, n is 57. In some embodiments, n is 58. In some embodiments, n is 59. In some embodiments, n is 60.

In some embodiments, the antigen expression domain 1 or antigen expression domain 2 is independently selectable from about 50 to about 10,000 nucleotides in length, for example about 50 to about 15,000 nucleotides in length, about 100 to about 15,000 nucleotides in length, about 500 to about 15,000 nucleotides in length, about 1,000 to about 15,000 nucleotides in length, about 5,000 to about 15,000 nucleotides in length, about 10,000 to about 15,000 nucleotides in length, and n is any positive integer from about 6 to about 26, for example about 6, about 7, about 8, about 9, about 10, about 11, about 12, about 13, about 14, about 15, about 16, about 17, about 18, about 19, about 20, about 21, about 22, about 23, about 24, about 25, or about 26.

In some embodiments, the antigen expression domain or the nucleic acid molecule comprises a nucleic acid sequence encoding β-catenin or a functional fragment thereof, wherein the nucleic acid sequence comprises at least about 70%, 75%, 80%, 85%, 90%, 91%, 92%, 93%, 94%, 95%, 96%, 97%, 98%, 99% or 100% sequence identity to SEQ ID NO:1, wherein the functional fragment comprises at least about 70% sequence identity to SEQ ID NO:1 and is free of a nucleic acid sequence that comprises 100% SEQ ID NO:1. In some embodiments, the antigen expression domain comprises a nucleic acid sequence encoding β-catenin or a functional fragment thereof, wherein the nucleic acid sequence encoding the β-catenin comprises at least about 70%, 75%, 80%, 85%, 90%, 91%, 92%, 93%, 94%, 95%, 96%, 97%, 98%, 99% or 100% sequence identity to SEQ ID NO:1. In some embodiments, the antigen expression domain comprises a nucleic acid sequence encoding β-catenin or a functional fragment thereof, wherein the nucleic acid sequence encoding the β-catenin or a fragment thereof comprises at least about 70%, 75%, 80%, 85%, 90%, 91%, 92%, 93%, 94%, 95%, 96%, 97%, 98%, 99% sequence identity to SEQ ID NO:1; and wherein the antigen expression domain is free of a nucleic acid sequence that comprises 100% sequence identity to SEQ ID NO:1.

In some embodiments, the antigen expression domain comprises a nucleic acid encoding β-catenin or a functional fragment thereof, wherein the nucleic acid sequence comprises RNA and is at least about 70%, 75%, 80%, 85%, 90%, 91%, 92%, 93%, 94%, 95%, 96%, 97%, 98%, 99% or 100% sequence identity to SEQ ID NO:2, and wherein the functional fragment comprises at least about 70% sequence identity to SEQ ID NO:2 and is free of a nucleic acid sequence that comprises 100% SEQ ID NO:2. In some embodiments, the antigen expression domain comprises a nucleic acid sequence encoding β-catenin or a fragment thereof, wherein the nucleic acid sequence encoding the β-catenin or a fragment thereof comprises at least about 70%, 75%, 80%, 85%, 90%, 91%, 92%, 93%, 94%, 95%, 96%, 97%, 98%, 99% or 100% sequence identity to SEQ ID NO:2. In some embodiments, the antigen expression domain comprises a nucleic acid sequence encoding β-catenin or a fragment thereof, wherein the nucleic acid sequence encoding the β-catenin or a fragment thereof comprises at least about 70%, 75%, 80%, 85%, 90%, 91%, 92%, 93%, 94%, 95%, 96%, 97%, 98%, 99% sequence identity to SEQ ID NO: 2; and wherein the antigen expression domain is free of a nucleic acid sequence that comprises 100% sequence identity to SEQ ID NO:2.

In some embodiments, the antigen expression domain comprises a nucleic acid encoding β-catenin or a functional fragment thereof, wherein the β-catenin or a functional fragment thereof comprises at least about 70%, 75%, 80%, 85%, 90%, 91%, 92%, 93%, 94%, 95%, 96%, 97%, 98%, 99% or 100% sequence identity to SEQ ID NO:13, and wherein the functional fragment comprises at least about 70% sequence identity to SEQ ID NO: 13 and is free of a nucleic acid sequence that comprises 100% SEQ ID NO:13. In some embodiments, the antigen expression domain comprises a nucleic acid sequence encoding β-catenin or a functional fragment thereof, wherein the β-catenin or a functional fragment thereof comprises at least about 70%, 75%, 80%, 85%, 90%, 91%, 92%, 93%, 94%, 95%, 96%, 97%, 98%, 99% or 100% sequence identity to SEQ ID NO:13. In some embodiments, the antigen expression domain comprises a nucleic acid sequence encoding β-catenin or a functional fragment thereof, wherein the β-catenin or a functional fragment thereof comprises at least about 70%, 75%, 80%, 85%, 90%, 91%, 92%, 93%, 94%, 95%, 96%, 97%, 98%, 99% sequence identity to SEQ ID NO:13; and wherein the antigen expression domain is free of a nucleic acid sequence that encodes an amino acid that comprises 100% sequence identity to SEQ ID NO: 13.

In some embodiments, the antigen expression domain or the nucleic acid molecule comprises a nucleic acid sequence encoding AXIN1 or a functional fragment thereof, wherein the nucleic acid sequence comprises at least about 70%, 75%, 80%, 85%, 90%, 91%, 92%, 93%, 94%, 95%, 96%, 97%, 98%, 99% or 100% sequence identity to SEQ ID NO:3, wherein the functional fragment comprises at least about 70% sequence identity to SEQ ID NO:3 and is free of a nucleic acid sequence that comprises 100% SEQ ID NO:3. In some embodiments, the antigen expression domain comprises a nucleic acid sequence encoding AXIN1 or a functional fragment thereof, wherein the nucleic acid sequence encoding the AXIN1 comprises at least about 70%, 75%, 80%, 85%, 90%, 91%, 92%, 93%, 94%, 95%, 96%, 97%, 98%, 99% or 100% sequence identity to SEQ ID NO:3. In some embodiments, the antigen expression domain comprises a nucleic acid sequence encoding AXIN1 or a functional fragment thereof, wherein the nucleic acid sequence encoding the AXIN1 or a fragment thereof comprises at least about 70%, 75%, 80%, 85%, 90%, 91%, 92%, 93%, 94%, 95%, 96%, 97%, 98%, 99% sequence identity to SEQ ID NO:3; and wherein the antigen expression domain is free of a nucleic acid sequence that comprises 100% sequence identity to SEQ ID NO:3.

In some embodiments, the antigen expression domain comprises a nucleic acid encoding AXIN1 or a functional fragment thereof, wherein the nucleic acid sequence comprises RNA and is at least about 70%, 75%, 80%, 85%, 90%, 91%, 92%, 93%, 94%, 95%, 96%, 97%, 98%, 99% or 100% sequence identity to SEQ ID NO:4, and wherein the functional fragment comprises at least about 70% sequence identity to SEQ ID NO:4 and is free of a nucleic acid sequence that comprises 100% SEQ ID NO:4. In some embodiments, the antigen expression domain comprises a nucleic acid sequence encoding AXIN1 or a fragment thereof, wherein the nucleic acid sequence encoding the AXIN1 or a fragment thereof comprises at least about 70%, 75%, 80%, 85%, 90%, 91%, 92%, 93%, 94%, 95%, 96%, 97%, 98%, 99% or 100% sequence identity to SEQ ID NO:4. In some embodiments, the antigen expression domain comprises a nucleic acid sequence encoding AXIN1 or a fragment thereof, wherein the nucleic acid sequence encoding the AXIN1 or a fragment thereof comprises at least about 70%, 75%, 80%, 85%, 90%, 91%, 92%, 93%, 94%, 95%, 96%, 97%, 98%, 99% sequence identity to SEQ ID NO:4; and wherein the antigen expression domain is free of a nucleic acid sequence that comprises 100% sequence identity to SEQ ID NO:4.

In some embodiments, the antigen expression domain comprises a nucleic acid encoding AXIN1 or a functional fragment thereof, wherein the AXIN1 or a functional fragment thereof comprises at least about 70%, 75%, 80%, 85%, 90%, 91%, 92%, 93%, 94%, 95%, 96%, 97%, 98%, 99% or 100% sequence identity to SEQ ID NO:14, and wherein the functional fragment comprises at least about 70% sequence identity to SEQ ID NO: 14 and is free of a nucleic acid sequence that comprises 100% SEQ ID NO:14. In some embodiments, the antigen expression domain comprises a nucleic acid sequence encoding AXIN1 or a functional fragment thereof, wherein the AXIN1 or a functional fragment thereof comprises at least about 70%, 75%, 80%, 85%, 90%, 91%, 92%, 93%, 94%, 95%, 96%, 97%, 98%, 99% or 100% sequence identity to SEQ ID NO:14. In some embodiments, the antigen expression domain comprises a nucleic acid sequence encoding AXIN1 or a functional fragment thereof, wherein the AXIN1 or a functional fragment thereof comprises at least about 70%, 75%, 80%, 85%, 90%, 91%, 92%, 93%, 94%, 95%, 96%, 97%, 98%, 99% sequence identity to SEQ ID NO:14; and wherein the antigen expression domain is free of a nucleic acid sequence that encodes an amino acid that comprises 100% sequence identity to SEQ ID NO:14.

In some embodiments, the antigen expression domain or the nucleic acid molecule comprises a nucleic acid sequence encoding AXIN2 or a functional fragment thereof, wherein the nucleic acid sequence comprises at least about 70%, 75%, 80%, 85%, 90%, 91%, 92%, 93%, 94%, 95%, 96%, 97%, 98%, 99% or 100% sequence identity to SEQ ID NO:5, wherein the functional fragment comprises at least about 70% sequence identity to SEQ ID NO:5 and is free of a nucleic acid sequence that comprises 100% SEQ ID NO:5. In some embodiments, the antigen expression domain comprises a nucleic acid sequence encoding AXIN2 or a functional fragment thereof, wherein the nucleic acid sequence encoding the AXIN2 comprises at least about 70%, 75%, 80%, 85%, 90%, 91%, 92%, 93%, 94%, 95%, 96%, 97%, 98%, 99% or 100% sequence identity to SEQ ID NO:5. In some embodiments, the antigen expression domain comprises a nucleic acid sequence encoding AXIN2 or a functional fragment thereof, wherein the nucleic acid sequence encoding the AXIN2 or a fragment thereof comprises at least about 70%, 75%, 80%, 85%, 90%, 91%, 92%, 93%, 94%, 95%, 96%, 97%, 98%, 99% sequence identity to SEQ ID NO:5; and wherein the antigen expression domain is free of a nucleic acid sequence that comprises 100% sequence identity to SEQ ID NO:5.

In some embodiments, the antigen expression domain comprises a nucleic acid encoding AXIN2 or a functional fragment thereof, wherein the nucleic acid sequence comprises RNA and is at least about 70%, 75%, 80%, 85%, 90%, 91%, 92%, 93%, 94%, 95%, 96%, 97%, 98%, 99% or 100% sequence identity to SEQ ID NO:6, and wherein the functional fragment comprises at least about 70% sequence identity to SEQ ID NO:6 and is free of a nucleic acid sequence that comprises 100% SEQ ID NO:6. In some embodiments, the antigen expression domain comprises a nucleic acid sequence encoding AXIN2 or a fragment thereof, wherein the nucleic acid sequence encoding the AXIN2 or a fragment thereof comprises at least about 70%, 75%, 80%, 85%, 90%, 91%, 92%, 93%, 94%, 95%, 96%, 97%, 98%, 99% or 100% sequence identity to SEQ ID NO:6. In some embodiments, the antigen expression domain comprises a nucleic acid sequence encoding AXIN2 or a fragment thereof, wherein the nucleic acid sequence encoding the AXIN2 or a fragment thereof comprises at least about 70%, 75%, 80%, 85%, 90%, 91%, 92%, 93%, 94%, 95%, 96%, 97%, 98%, 99% sequence identity to SEQ ID NO:6; and wherein the antigen expression domain is free of a nucleic acid sequence that comprises 100% sequence identity to SEQ ID NO:6.

In some embodiments, the antigen expression domain comprises a nucleic acid encoding AXIN2 or a functional fragment thereof, wherein the AXIN2 or a functional fragment thereof comprises at least about 70%, 75%, 80%, 85%, 90%, 91%, 92%, 93%, 94%, 95%, 96%, 97%, 98%, 99% or 100% sequence identity to SEQ ID NO:15, and wherein the functional fragment comprises at least about 70% sequence identity to SEQ ID NO:15 and is free of a nucleic acid sequence that comprises 100% SEQ ID NO:15. In some embodiments, the antigen expression domain comprises a nucleic acid sequence encoding AXIN2 or a functional fragment thereof, wherein the AXIN2 or a functional fragment thereof comprises at least about 70%, 75%, 80%, 85%, 90%, 91%, 92%, 93%, 94%, 95%, 96%, 97%, 98%, 99% or 100% sequence identity to SEQ ID NO:15. In some embodiments, the antigen expression domain comprises a nucleic acid sequence encoding AXIN2 or a functional fragment thereof, wherein the AXIN2 or a functional fragment thereof comprises at least about 70%, 75%, 80%, 85%, 90%, 91%, 92%, 93%, 94%, 95%, 96%, 97%, 98%, 99% sequence identity to SEQ ID NO:15; and wherein the antigen expression domain is free of a nucleic acid sequence that encodes an amino acid that comprises 100% sequence identity to SEQ ID NO:15.

In some embodiments, the antigen expression domain or the nucleic acid molecule comprises a nucleic acid sequence encoding APC or a functional fragment thereof, wherein the nucleic acid sequence comprises at least about 70%, 75%, 80%, 85%, 90%, 91%, 92%, 93%, 94%, 95%, 96%, 97%, 98%, 99% or 100% sequence identity to SEQ ID NO:7, wherein the functional fragment comprises at least about 70% sequence identity to SEQ ID NO:7 and is free of a nucleic acid sequence that comprises 100% SEQ ID NO:7. In some embodiments, the antigen expression domain comprises a nucleic acid sequence encoding APC or a functional fragment thereof, wherein the nucleic acid sequence encoding the APC comprises at least about 70%, 75%, 80%, 85%, 90%, 91%, 92%, 93%, 94%, 95%, 96%, 97%, 98%, 99% or 100% sequence identity to SEQ ID NO:7. In some embodiments, the antigen expression domain comprises a nucleic acid sequence encoding APC or a functional fragment thereof, wherein the nucleic acid sequence encoding the APC or a fragment thereof comprises at least about 70%, 75%, 80%, 85%, 90%, 91%, 92%, 93%, 94%, 95%, 96%, 97%, 98%, 99% sequence identity to SEQ ID NO:7; and wherein the antigen expression domain is free of a nucleic acid sequence that comprises 100% sequence identity to SEQ ID NO:7.

In some embodiments, the antigen expression domain comprises a nucleic acid encoding APC or a functional fragment thereof, wherein the nucleic acid sequence comprises RNA and is at least about 70%, 75%, 80%, 85%, 90%, 91%, 92%, 93%, 94%, 95%, 96%, 97%, 98%, 99% or 100% sequence identity to SEQ ID NO:8, and wherein the functional fragment comprises at least about 70% sequence identity to SEQ ID NO:8 and is free of a nucleic acid sequence that comprises 100% SEQ ID NO:8. In some embodiments, the antigen expression domain comprises a nucleic acid sequence encoding APC or a fragment thereof, wherein the nucleic acid sequence encoding the APC or a fragment thereof comprises at least about 70%, 75%, 80%, 85%, 90%, 91%, 92%, 93%, 94%, 95%, 96%, 97%, 98%, 99% or 100% sequence identity to SEQ ID NO:8. In some embodiments, the antigen expression domain comprises a nucleic acid sequence encoding APC or a fragment thereof, wherein the nucleic acid sequence encoding the APC or a fragment thereof comprises at least about 70%, 75%, 80%, 85%, 90%, 91%, 92%, 93%, 94%, 95%, 96%, 97%, 98%, 99% sequence identity to SEQ ID NO:8; and wherein the antigen expression domain is free of a nucleic acid sequence that comprises 100% sequence identity to SEQ ID NO:8.

In some embodiments, the antigen expression domain comprises a nucleic acid encoding APC or a functional fragment thereof, wherein the APC or a functional fragment thereof comprises at least about 70%, 75%, 80%, 85%, 90%, 91%, 92%, 93%, 94%, 95%, 96%, 97%, 98%, 99% or 100% sequence identity to SEQ ID NO:16, and wherein the functional fragment comprises at least about 70% sequence identity to SEQ ID NO:16 and is free of a nucleic acid sequence that comprises 100% SEQ ID NO:16. In some embodiments, the antigen expression domain comprises a nucleic acid sequence encoding APC or a functional fragment thereof, wherein the APC or a functional fragment thereof comprises at least about 70%, 75%, 80%, 85%, 90%, 91%, 92%, 93%, 94%, 95%, 96%, 97%, 98%, 99% or 100% sequence identity to SEQ ID NO:16. In some embodiments, the antigen expression domain comprises a nucleic acid sequence encoding APC or a functional fragment thereof, wherein the APC or a functional fragment thereof comprises at least about 70%, 75%, 80%, 85%, 90%, 91%, 92%, 93%, 94%, 95%, 96%, 97%, 98%, 99% sequence identity to SEQ ID NO:16; and wherein the antigen expression domain is free of a nucleic acid sequence that encodes an amino acid that comprises 100% sequence identity to SEQ ID NO:16.

In some embodiments, the antigen expression domain or the nucleic acid molecule comprises a nucleic acid sequence encoding CSNK1A1 or a functional fragment thereof, wherein the nucleic acid sequence comprises at least about 70%, 75%, 80%, 85%, 90%, 91%, 92%, 93%, 94%, 95%, 96%, 97%, 98%, 99% or 100% sequence identity to SEQ ID NO:9, wherein the functional fragment comprises at least about 70% sequence identity to SEQ ID NO:9 and is free of a nucleic acid sequence that comprises 100% SEQ ID NO:9. In some embodiments, the antigen expression domain comprises a nucleic acid sequence encoding CSNK1A1 or a functional fragment thereof, wherein the nucleic acid sequence encoding the CSNK1A1 comprises at least about 70%, 75%, 80%, 85%, 90%, 91%, 92%, 93%, 94%, 95%, 96%, 97%, 98%, 99% or 100% sequence identity to SEQ ID NO:9. In some embodiments, the antigen expression domain comprises a nucleic acid sequence encoding CSNK1A1 or a functional fragment thereof, wherein the nucleic acid sequence encoding the CSNK1A1 or a fragment thereof comprises at least about 70%, 75%, 80%, 85%, 90%, 91%, 92%, 93%, 94%, 95%, 96%, 97%, 98%, 99% sequence identity to SEQ ID NO:9; and wherein the antigen expression domain is free of a nucleic acid sequence that comprises 100% sequence identity to SEQ ID NO:9.

In some embodiments, the antigen expression domain comprises a nucleic acid encoding CSNK1A1 or a functional fragment thereof, wherein the nucleic acid sequence comprises RNA and is at least about 70%, 75%, 80%, 85%, 90%, 91%, 92%, 93%, 94%, 95%, 96%, 97%, 98%, 99% or 100% sequence identity to SEQ ID NO: 10, and wherein the functional fragment comprises at least about 70% sequence identity to SEQ ID NO: 10 and is free of a nucleic acid sequence that comprises 100% SEQ ID NO:10. In some embodiments, the antigen expression domain comprises a nucleic acid sequence encoding CSNK1A1 or a fragment thereof, wherein the nucleic acid sequence encoding the CSNK1A1 or a fragment thereof comprises at least about 70%, 75%, 80%, 85%, 90%, 91%, 92%, 93%, 94%, 95%, 96%, 97%, 98%, 99% or 100% sequence identity to SEQ ID NO:10. In some embodiments, the antigen expression domain comprises a nucleic acid sequence encoding CSNK1A1 or a fragment thereof, wherein the nucleic acid sequence encoding the CSNK1A1 or a fragment thereof comprises at least about 70%, 75%, 80%, 85%, 90%, 91%, 92%, 93%, 94%, 95%, 96%, 97%, 98%, 99% sequence identity to SEQ ID NO:10; and wherein the antigen expression domain is free of a nucleic acid sequence that comprises 100% sequence identity to SEQ ID NO: 10.

In some embodiments, the antigen expression domain comprises a nucleic acid encoding CSNK1A1 or a functional fragment thereof, wherein the CSNK1A1 or a functional fragment thereof comprises at least about 70%, 75%, 80%, 85%, 90%, 91%, 92%, 93%, 94%, 95%, 96%, 97%, 98%, 99% or 100% sequence identity to SEQ ID NO:17, and wherein the functional fragment comprises at least about 70% sequence identity to SEQ ID NO: 17 and is free of a nucleic acid sequence that comprises 100% SEQ ID NO:17. In some embodiments, the antigen expression domain comprises a nucleic acid sequence encoding CSNK1A1 or a functional fragment thereof, wherein the CSNK1A1 or a functional fragment thereof comprises at least about 70%, 75%, 80%, 85%, 90%, 91%, 92%, 93%, 94%, 95%, 96%, 97%, 98%, 99% or 100% sequence identity to SEQ ID NO:17. In some embodiments, the antigen expression domain comprises a nucleic acid sequence encoding CSNK1A1 or a functional fragment thereof, wherein the CSNK1A1 or a functional fragment thereof comprises at least about 70%, 75%, 80%, 85%, 90%, 91%, 92%, 93%, 94%, 95%, 96%, 97%, 98%, 99% sequence identity to SEQ ID NO: 17; and wherein the antigen expression domain is free of a nucleic acid sequence that encodes an amino acid that comprises 100% sequence identity to SEQ ID NO:17.

In some embodiments, the antigen expression domain or the nucleic acid molecule comprises a nucleic acid sequence encoding GSK3B or a functional fragment thereof, wherein the nucleic acid sequence comprises at least about 70%, 75%, 80%, 85%, 90%, 91%, 92%, 93%, 94%, 95%, 96%, 97%, 98%, 99% or 100% sequence identity to SEQ ID NO:11, wherein the functional fragment comprises at least about 70% sequence identity to SEQ ID NO:11 and is free of a nucleic acid sequence that comprises 100% SEQ ID NO:11. In some embodiments, the antigen expression domain comprises a nucleic acid sequence encoding GSK3B or a functional fragment thereof, wherein the nucleic acid sequence encoding the GSK3B comprises at least about 70%, 75%, 80%, 85%, 90%, 91%, 92%, 93%, 94%, 95%, 96%, 97%, 98%, 99% or 100% sequence identity to SEQ ID NO:11. In some embodiments, the antigen expression domain comprises a nucleic acid sequence encoding GSK3B or a functional fragment thereof, wherein the nucleic acid sequence encoding the GSK3B or a fragment thereof comprises at least about 70%, 75%, 80%, 85%, 90%, 91%, 92%, 93%, 94%, 95%, 96%, 97%, 98%, 99% sequence identity to SEQ ID NO:11; and wherein the antigen expression domain is free of a nucleic acid sequence that comprises 100% sequence identity to SEQ ID NO:11.

In some embodiments, the antigen expression domain comprises a nucleic acid encoding GSK3B or a functional fragment thereof, wherein the nucleic acid sequence comprises RNA and is at least about 70%, 75%, 80%, 85%, 90%, 91%, 92%, 93%, 94%, 95%, 96%, 97%, 98%, 99% or 100% sequence identity to SEQ ID NO:12, and wherein the functional fragment comprises at least about 70% sequence identity to SEQ ID NO: 12 and is free of a nucleic acid sequence that comprises 100% SEQ ID NO:12. In some embodiments, the antigen expression domain comprises a nucleic acid sequence encoding GSK3B or a fragment thereof, wherein the nucleic acid sequence encoding the GSK3B or a fragment thereof comprises at least about 70%, 75%, 80%, 85%, 90%, 91%, 92%, 93%, 94%, 95%, 96%, 97%, 98%, 99% or 100% sequence identity to SEQ ID NO:12. In some embodiments, the antigen expression domain comprises a nucleic acid sequence encoding GSK3B or a fragment thereof, wherein the nucleic acid sequence encoding the GSK3B or a fragment thereof comprises at least about 70%, 75%, 80%, 85%, 90%, 91%, 92%, 93%, 94%, 95%, 96%, 97%, 98%, 99% sequence identity to SEQ ID NO:12; and wherein the antigen expression domain is free of a nucleic acid sequence that comprises 100% sequence identity to SEQ ID NO:12.

In some embodiments, the antigen expression domain comprises a nucleic acid encoding GSK3B or a functional fragment thereof, wherein the GSK3B or a functional fragment thereof comprises at least about 70%, 75%, 80%, 85%, 90%, 91%, 92%, 93%, 94%, 95%, 96%, 97%, 98%, 99% or 100% sequence identity to SEQ ID NO:18, and wherein the functional fragment comprises at least about 70% sequence identity to SEQ ID NO:18 and is free of a nucleic acid sequence that comprises 100% SEQ ID NO:18. In some embodiments, the antigen expression domain comprises a nucleic acid sequence encoding GSK3B or a functional fragment thereof, wherein the GSK3B or a functional fragment thereof comprises at least about 70%, 75%, 80%, 85%, 90%, 91%, 92%, 93%, 94%, 95%, 96%, 97%, 98%, 99% or 100% sequence identity to SEQ ID NO:18. In some embodiments, the antigen expression domain comprises a nucleic acid sequence encoding GSK3B or a functional fragment thereof, wherein the GSK3B or a functional fragment thereof comprises at least about 70%, 75%, 80%, 85%, 90%, 91%, 92%, 93%, 94%, 95%, 96%, 97%, 98%, 99% sequence identity to SEQ ID NO:18; and wherein the antigen expression domain is free of a nucleic acid sequence that encodes an amino acid that comprises 100% sequence identity to SEQ ID NO:18.

In some embodiments, the antigen expression domain or the nucleic acid molecule comprises a nucleic acid sequence encoding a functional fragment of β-catenin, wherein the nucleic acid sequence comprises at least about 70%, 75%, 80%, 85%, 90%, 91%, 92%, 93%, 94%, 95%, 96%, 97%, 98%, 99% or 100% sequence identity to SEQ ID NO:19, wherein the functional fragment comprises at least about 70% sequence identity to SEQ ID NO:19. In some embodiments, the antigen expression domain comprises a nucleic acid sequence encoding a functional fragment of β-catenin, wherein the nucleic acid sequence encoding the functional fragment comprises at least about 70%, 75%, 80%, 85%, 90%, 91%, 92%, 93%, 94%, 95%, 96%, 97%, 98%, 99% or 100% sequence identity to SEQ ID NO:19. In some embodiments, the antigen expression domain comprises a nucleic acid sequence encoding a functional fragment of β-catenin, wherein the nucleic acid sequence encoding the functional fragment of β-catenin comprises at least about 70%, 75%, 80%, 85%, 90%, 91%, 92%, 93%, 94%, 95%, 96%, 97%, 98%, 99% sequence identity to SEQ ID NO:19; and wherein the antigen expression domain is free of a nucleic acid sequence that comprises 100% sequence identity to SEQ ID NO:19.

In some embodiments, the antigen expression domain or the nucleic acid molecule comprises a nucleic acid sequence encoding a functional fragment of β-catenin, wherein the nucleic acid sequence comprises at least about 70%, 75%, 80%, 85%, 90%, 91%, 92%, 93%, 94%, 95%, 96%, 97%, 98%, 99% or 100% sequence identity to SEQ ID NO:20. In some embodiments, the antigen expression domain comprises a nucleic acid sequence encoding a functional fragment of β-catenin, wherein the nucleic acid sequence encoding the functional fragment comprises at least about 70%, 75%, 80%, 85%, 90%, 91%, 92%, 93%, 94%, 95%, 96%, 97%, 98%, 99% or 100% sequence identity to SEQ ID NO:20. In some embodiments, the antigen expression domain comprises a nucleic acid sequence encoding a functional fragment of β-catenin, wherein the nucleic acid sequence encoding the functional fragment of β-catenin comprises at least about 70%, 75%, 80%, 85%, 90%, 91%, 92%, 93%, 94%, 95%, 96%, 97%, 98%, 99% sequence identity to SEQ ID NO:20; and wherein the antigen expression domain is free of a nucleic acid sequence that comprises 100% sequence identity to SEQ ID NO:20.

In some embodiments, the nucleic acid molecule comprises an antigen expression domain comprising a nucleic acid sequence encoding a functional fragment of β-catenin, wherein the functional fragment thereof comprises at least about 70%, 75%, 80%, 85%, 90%, 91%, 92%, 93%, 94%, 95%, 96%, 97%, 98%, 99% or 100% sequence identity to SEQ ID NO:31, and wherein the functional fragment comprises at least about 70% sequence identity to SEQ ID NO: 31. In some embodiments, the antigen expression domain comprises a nucleic acid sequence encoding β-catenin or a functional fragment thereof, wherein the β-catenin or a functional fragment thereof comprises at least about 70%, 75%, 80%, 85%, 90%, 91%, 92%, 93%, 94%, 95%, 96%, 97%, 98%, 99% or 100% sequence identity to SEQ ID NO:31. In some embodiments, the antigen expression domain comprises a nucleic acid sequence encoding β-catenin or a functional fragment thereof, wherein the β-catenin or a functional fragment thereof comprises at least about 70%, 75%, 80%, 85%, 90%, 91%, 92%, 93%, 94%, 95%, 96%, 97%, 98%, 99% sequence identity to SEQ ID NO:31; and wherein the antigen expression domain is free of a nucleic acid sequence that encodes an amino acid that comprises 100% sequence identity to SEQ ID NO:31.

In some embodiments, the antigen expression domain or the nucleic acid molecule comprises a nucleic acid sequence encoding a functional fragment of β-catenin, wherein the nucleic acid sequence comprises at least about 70%, 75%, 80%, 85%, 90%, 91%, 92%, 93%, 94%, 95%, 96%, 97%, 98%, 99% or 100% sequence identity to SEQ ID NO:21, wherein the functional fragment comprises at least about 70% sequence identity to SEQ ID NO:21. In some embodiments, the antigen expression domain comprises a nucleic acid sequence encoding a functional fragment of β-catenin, wherein the nucleic acid sequence encoding the functional fragment comprises at least about 70%, 75%, 80%, 85%, 90%, 91%, 92%, 93%, 94%, 95%, 96%, 97%, 98%, 99% or 100% sequence identity to SEQ ID NO:21. In some embodiments, the antigen expression domain comprises a nucleic acid sequence encoding a functional fragment of β-catenin, wherein the nucleic acid sequence encoding the functional fragment of β-catenin comprises at least about 70%, 75%, 80%, 85%, 90%, 91%, 92%, 93%, 94%, 95%, 96%, 97%, 98%, 99% sequence identity to SEQ ID NO:21; and wherein the antigen expression domain is free of a nucleic acid sequence that comprises 100% sequence identity to SEQ ID NO:21.

In some embodiments, the antigen expression domain or the nucleic acid molecule comprises a nucleic acid sequence encoding a functional fragment of β-catenin, wherein the nucleic acid sequence comprises at least about 70%, 75%, 80%, 85%, 90%, 91%, 92%, 93%, 94%, 95%, 96%, 97%, 98%, 99% or 100% sequence identity to SEQ ID NO:22. In some embodiments, the antigen expression domain comprises a nucleic acid sequence encoding a functional fragment of β-catenin, wherein the nucleic acid sequence encoding the functional fragment comprises at least about 70%, 75%, 80%, 85%, 90%, 91%, 92%, 93%, 94%, 95%, 96%, 97%, 98%, 99% or 100% sequence identity to SEQ ID NO:22. In some embodiments, the antigen expression domain comprises a nucleic acid sequence encoding a functional fragment of β-catenin, wherein the nucleic acid sequence encoding the functional fragment of β-catenin comprises at least about 70%, 75%, 80%, 85%, 90%, 91%, 92%, 93%, 94%, 95%, 96%, 97%, 98%, 99% sequence identity to SEQ ID NO:22; and wherein the antigen expression domain is free of a nucleic acid sequence that comprises 100% sequence identity to SEQ ID NO:22.

In some embodiments, the nucleic acid molecule comprises an antigen expression domain comprising a nucleic acid sequence encoding a functional fragment of β-catenin, wherein the functional fragment thereof comprises at least about 70%, 75%, 80%, 85%, 90%, 91%, 92%, 93%, 94%, 95%, 96%, 97%, 98%, 99% or 100% sequence identity to SEQ ID NO:32, and wherein the functional fragment comprises at least about 70% sequence identity to SEQ ID NO: 32. In some embodiments, the antigen expression domain comprises a nucleic acid sequence encoding β-catenin or a functional fragment thereof, wherein the β-catenin or a functional fragment thereof comprises at least about 70%, 75%, 80%, 85%, 90%, 91%, 92%, 93%, 94%, 95%, 96%, 97%, 98%, 99% or 100% sequence identity to SEQ ID NO:32. In some embodiments, the antigen expression domain comprises a nucleic acid sequence encoding β-catenin or a functional fragment thereof, wherein the β-catenin or a functional fragment thereof comprises at least about 70%, 75%, 80%, 85%, 90%, 91%, 92%, 93%, 94%, 95%, 96%, 97%, 98%, 99% sequence identity to SEQ ID NO:32; and wherein the antigen expression domain is free of a nucleic acid sequence that encodes an amino acid that comprises 100% sequence identity to SEQ ID NO:32.

In some embodiments, the antigen expression domain or the nucleic acid molecule comprises a nucleic acid sequence encoding a functional fragment of AXIN1, wherein the nucleic acid sequence comprises at least about 70%, 75%, 80%, 85%, 90%, 91%, 92%, 93%, 94%, 95%, 96%, 97%, 98%, 99% or 100% sequence identity to SEQ ID NO:23, wherein the functional fragment comprises at least about 70% sequence identity to SEQ ID NO:23. In some embodiments, the antigen expression domain comprises a nucleic acid sequence encoding a functional fragment of AXIN1, wherein the nucleic acid sequence encoding the functional fragment comprises at least about 70%, 75%, 80%, 85%, 90%, 91%, 92%, 93%, 94%, 95%, 96%, 97%, 98%, 99% or 100% sequence identity to SEQ ID NO:23. In some embodiments, the antigen expression domain comprises a nucleic acid sequence encoding a functional fragment of AXIN1, wherein the nucleic acid sequence encoding the functional fragment of AXIN1 comprises at least about 70%, 75%, 80%, 85%, 90%, 91%, 92%, 93%, 94%, 95%, 96%, 97%, 98%, 99% sequence identity to SEQ ID NO:23; and wherein the antigen expression domain is free of a nucleic acid sequence that comprises 100% sequence identity to SEQ ID NO:23.

In some embodiments, the antigen expression domain or the nucleic acid molecule comprises a nucleic acid sequence encoding a functional fragment of AXIN1, wherein the nucleic acid sequence comprises at least about 70%, 75%, 80%, 85%, 90%, 91%, 92%, 93%, 94%, 95%, 96%, 97%, 98%, 99% or 100% sequence identity to SEQ ID NO:24. In some embodiments, the antigen expression domain comprises a nucleic acid sequence encoding a functional fragment of AXIN1, wherein the nucleic acid sequence encoding the functional fragment comprises at least about 70%, 75%, 80%, 85%, 90%, 91%, 92%, 93%, 94%, 95%, 96%, 97%, 98%, 99% or 100% sequence identity to SEQ ID NO:24. In some embodiments, the antigen expression domain comprises a nucleic acid sequence encoding a functional fragment of AXIN1, wherein the nucleic acid sequence encoding the functional fragment of AXIN1 comprises at least about 70%, 75%, 80%, 85%, 90%, 91%, 92%, 93%, 94%, 95%, 96%, 97%, 98%, 99% sequence identity to SEQ ID NO:24; and wherein the antigen expression domain is free of a nucleic acid sequence that comprises 100% sequence identity to SEQ ID NO:24.

In some embodiments, the nucleic acid molecule comprises an antigen expression domain comprising a nucleic acid sequence encoding a functional fragment of AXIN1, wherein the functional fragment thereof comprises at least about 70%, 75%, 80%, 85%, 90%, 91%, 92%, 93%, 94%, 95%, 96%, 97%, 98%, 99% or 100% sequence identity to SEQ ID NO:33, and wherein the functional fragment comprises at least about 70% sequence identity to SEQ ID NO: 33. In some embodiments, the antigen expression domain comprises a nucleic acid sequence encoding AXIN1 or a functional fragment thereof, wherein the AXIN1 or a functional fragment thereof comprises at least about 70%, 75%, 80%, 85%, 90%, 91%, 92%, 93%, 94%, 95%, 96%, 97%, 98%, 99% or 100% sequence identity to SEQ ID NO:33. In some embodiments, the antigen expression domain comprises a nucleic acid sequence encoding AXIN1 or a functional fragment thereof, wherein the AXIN1 or a functional fragment thereof comprises at least about 70%, 75%, 80%, 85%, 90%, 91%, 92%, 93%, 94%, 95%, 96%, 97%, 98%, 99% sequence identity to SEQ ID NO:33; and wherein the antigen expression domain is free of a nucleic acid sequence that encodes an amino acid that comprises 100% sequence identity to SEQ ID NO:33.

In some embodiments, the antigen expression domain or the nucleic acid molecule comprises a nucleic acid sequence encoding a functional fragment of β-catenin, wherein the nucleic acid sequence comprises at least about 70%, 75%, 80%, 85%, 90%, 91%, 92%, 93%, 94%, 95%, 96%, 97%, 98%, 99% or 100% sequence identity to SEQ ID NO:25, wherein the functional fragment comprises at least about 70% sequence identity to SEQ ID NO:25. In some embodiments, the antigen expression domain comprises a nucleic acid sequence encoding a functional fragment of β-catenin, wherein the nucleic acid sequence encoding the functional fragment comprises at least about 70%, 75%, 80%, 85%, 90%, 91%, 92%, 93%, 94%, 95%, 96%, 97%, 98%, 99% or 100% sequence identity to SEQ ID NO:25. In some embodiments, the antigen expression domain comprises a nucleic acid sequence encoding a functional fragment of β-catenin, wherein the nucleic acid sequence encoding the functional fragment of β-catenin comprises at least about 70%, 75%, 80%, 85%, 90%, 91%, 92%, 93%, 94%, 95%, 96%, 97%, 98%, 99% sequence identity to SEQ ID NO:25; and wherein the antigen expression domain is free of a nucleic acid sequence that comprises 100% sequence identity to SEQ ID NO:25.

In some embodiments, the antigen expression domain or the nucleic acid molecule comprises a nucleic acid sequence encoding a functional fragment of β-catenin, wherein the nucleic acid sequence comprises at least about 70%, 75%, 80%, 85%, 90%, 91%, 92%, 93%, 94%, 95%, 96%, 97%, 98%, 99% or 100% sequence identity to SEQ ID NO:26. In some embodiments, the antigen expression domain comprises a nucleic acid sequence encoding a functional fragment of β-catenin, wherein the nucleic acid sequence encoding the functional fragment comprises at least about 70%, 75%, 80%, 85%, 90%, 91%, 92%, 93%, 94%, 95%, 96%, 97%, 98%, 99% or 100% sequence identity to SEQ ID NO:26. In some embodiments, the antigen expression domain comprises a nucleic acid sequence encoding a functional fragment of β-catenin, wherein the nucleic acid sequence encoding the functional fragment of β-catenin comprises at least about 70%, 75%, 80%, 85%, 90%, 91%, 92%, 93%, 94%, 95%, 96%, 97%, 98%, 99% sequence identity to SEQ ID NO:26; and wherein the antigen expression domain is free of a nucleic acid sequence that comprises 100% sequence identity to SEQ ID NO:26.

In some embodiments, the nucleic acid molecule comprises an antigen expression domain comprising a nucleic acid sequence encoding a functional fragment of β-catenin, wherein the functional fragment thereof comprises at least about 70%, 75%, 80%, 85%, 90%, 91%, 92%, 93%, 94%, 95%, 96%, 97%, 98%, 99% or 100% sequence identity to SEQ ID NO:34, and wherein the functional fragment comprises at least about 70% sequence identity to SEQ ID NO: 34. In some embodiments, the antigen expression domain comprises a nucleic acid sequence encoding β-catenin or a functional fragment thereof, wherein the β-catenin or a functional fragment thereof comprises at least about 70%, 75%, 80%, 85%, 90%, 91%, 92%, 93%, 94%, 95%, 96%, 97%, 98%, 99% or 100% sequence identity to SEQ ID NO:34. In some embodiments, the antigen expression domain comprises a nucleic acid sequence encoding β-catenin or a functional fragment thereof, wherein the β-catenin or a functional fragment thereof comprises at least about 70%, 75%, 80%, 85%, 90%, 91%, 92%, 93%, 94%, 95%, 96%, 97%, 98%, 99% sequence identity to SEQ ID NO:34; and wherein the antigen expression domain is free of a nucleic acid sequence that encodes an amino acid that comprises 100% sequence identity to SEQ ID NO:34.

In some embodiments, the antigen expression domain or the nucleic acid molecule comprises a nucleic acid sequence encoding a functional fragment of β-catenin, wherein the nucleic acid sequence comprises at least about 70%, 75%, 80%, 85%, 90%, 91%, 92%, 93%, 94%, 95%, 96%, 97%, 98%, 99% or 100% sequence identity to SEQ ID NO:27, wherein the functional fragment comprises at least about 70% sequence identity to SEQ ID NO:27. In some embodiments, the antigen expression domain comprises a nucleic acid sequence encoding a functional fragment of β-catenin, wherein the nucleic acid sequence encoding the functional fragment comprises at least about 70%, 75%, 80%, 85%, 90%, 91%, 92%, 93%, 94%, 95%, 96%, 97%, 98%, 99% or 100% sequence identity to SEQ ID NO:27. In some embodiments, the antigen expression domain comprises a nucleic acid sequence encoding a functional fragment of β-catenin, wherein the nucleic acid sequence encoding the functional fragment of β-catenin comprises at least about 70%, 75%, 80%, 85%, 90%, 91%, 92%, 93%, 94%, 95%, 96%, 97%, 98%, 99% sequence identity to SEQ ID NO:27; and wherein the antigen expression domain is free of a nucleic acid sequence that comprises 100% sequence identity to SEQ ID NO:27.

In some embodiments, the antigen expression domain or the nucleic acid molecule comprises a nucleic acid sequence encoding a functional fragment of β-catenin, wherein the nucleic acid sequence comprises at least about 70%, 75%, 80%, 85%, 90%, 91%, 92%, 93%, 94%, 95%, 96%, 97%, 98%, 99% or 100% sequence identity to SEQ ID NO:28. In some embodiments, the antigen expression domain comprises a nucleic acid sequence encoding a functional fragment of β-catenin, wherein the nucleic acid sequence encoding the functional fragment comprises at least about 70%, 75%, 80%, 85%, 90%, 91%, 92%, 93%, 94%, 95%, 96%, 97%, 98%, 99% or 100% sequence identity to SEQ ID NO:28. In some embodiments, the antigen expression domain comprises a nucleic acid sequence encoding a functional fragment of β-catenin, wherein the nucleic acid sequence encoding the functional fragment of β-catenin comprises at least about 70%, 75%, 80%, 85%, 90%, 91%, 92%, 93%, 94%, 95%, 96%, 97%, 98%, 99% sequence identity to SEQ ID NO:28; and wherein the antigen expression domain is free of a nucleic acid sequence that comprises 100% sequence identity to SEQ ID NO:28.

In some embodiments, the nucleic acid molecule comprises an antigen expression domain comprising a nucleic acid sequence encoding a functional fragment of β-catenin, wherein the functional fragment thereof comprises at least about 70%, 75%, 80%, 85%, 90%, 91%, 92%, 93%, 94%, 95%, 96%, 97%, 98%, 99% or 100% sequence identity to SEQ ID NO:35, and wherein the functional fragment comprises at least about 70% sequence identity to SEQ ID NO: 35. In some embodiments, the antigen expression domain comprises a nucleic acid sequence encoding β-catenin or a functional fragment thereof, wherein the β-catenin or a functional fragment thereof comprises at least about 70%, 75%, 80%, 85%, 90%, 91%, 92%, 93%, 94%, 95%, 96%, 97%, 98%, 99% or 100% sequence identity to SEQ ID NO:35. In some embodiments, the antigen expression domain comprises a nucleic acid sequence encoding β-catenin or a functional fragment thereof, wherein the β-catenin or a functional fragment thereof comprises at least about 70%, 75%, 80%, 85%, 90%, 91%, 92%, 93%, 94%, 95%, 96%, 97%, 98%, 99% sequence identity to SEQ ID NO:35; and wherein the antigen expression domain is free of a nucleic acid sequence that encodes an amino acid that comprises 100% sequence identity to SEQ ID NO:35.

In some embodiments, the antigen expression domain or the nucleic acid molecule comprises a nucleic acid sequence encoding a functional fragment of β-catenin, wherein the nucleic acid sequence comprises at least about 70%, 75%, 80%, 85%, 90%, 91%, 92%, 93%, 94%, 95%, 96%, 97%, 98%, 99% or 100% sequence identity to SEQ ID NO:29, wherein the functional fragment comprises at least about 70% sequence identity to SEQ ID NO:29. In some embodiments, the antigen expression domain comprises a nucleic acid sequence encoding a functional fragment of β-catenin, wherein the nucleic acid sequence encoding the functional fragment comprises at least about 70%, 75%, 80%, 85%, 90%, 91%, 92%, 93%, 94%, 95%, 96%, 97%, 98%, 99% or 100% sequence identity to SEQ ID NO:29. In some embodiments, the antigen expression domain comprises a nucleic acid sequence encoding a functional fragment of β-catenin, wherein the nucleic acid sequence encoding the functional fragment of β-catenin comprises at least about 70%, 75%, 80%, 85%, 90%, 91%, 92%, 93%, 94%, 95%, 96%, 97%, 98%, 99% sequence identity to SEQ ID NO:29; and wherein the antigen expression domain is free of a nucleic acid sequence that comprises 100% sequence identity to SEQ ID NO:29.

In some embodiments, the antigen expression domain or the nucleic acid molecule comprises a nucleic acid sequence encoding a functional fragment of β-catenin, wherein the nucleic acid sequence comprises at least about 70%, 75%, 80%, 85%, 90%, 91%, 92%, 93%, 94%, 95%, 96%, 97%, 98%, 99% or 100% sequence identity to SEQ ID NO:30. In some embodiments, the antigen expression domain comprises a nucleic acid sequence encoding a functional fragment of β-catenin, wherein the nucleic acid sequence encoding the functional fragment comprises at least about 70%, 75%, 80%, 85%, 90%, 91%, 92%, 93%, 94%, 95%, 96%, 97%, 98%, 99% or 100% sequence identity to SEQ ID NO:30. In some embodiments, the antigen expression domain comprises a nucleic acid sequence encoding a functional fragment of β-catenin, wherein the nucleic acid sequence encoding the functional fragment of β-catenin comprises at least about 70%, 75%, 80%, 85%, 90%, 91%, 92%, 93%, 94%, 95%, 96%, 97%, 98%, 99% sequence identity to SEQ ID NO:30; and wherein the antigen expression domain is free of a nucleic acid sequence that comprises 100% sequence identity to SEQ ID NO:30.

In some embodiments, the nucleic acid molecule comprises an antigen expression domain comprising a nucleic acid sequence encoding a functional fragment of β-catenin, wherein the functional fragment thereof comprises at least about 70%, 75%, 80%, 85%, 90%, 91%, 92%, 93%, 94%, 95%, 96%, 97%, 98%, 99% or 100% sequence identity to SEQ ID NO:36, and wherein the functional fragment comprises at least about 70% sequence identity to SEQ ID NO: 36. In some embodiments, the antigen expression domain comprises a nucleic acid sequence encoding β-catenin or a functional fragment thereof, wherein the β-catenin or a functional fragment thereof comprises at least about 70%, 75%, 80%, 85%, 90%, 91%, 92%, 93%, 94%, 95%, 96%, 97%, 98%, 99% or 100% sequence identity to SEQ ID NO:36. In some embodiments, the antigen expression domain comprises a nucleic acid sequence encoding β-catenin or a functional fragment thereof, wherein the β-catenin or a functional fragment thereof comprises at least about 70%, 75%, 80%, 85%, 90%, 91%, 92%, 93%, 94%, 95%, 96%, 97%, 98%, 99% sequence identity to SEQ ID NO:36; and wherein the antigen expression domain is free of a nucleic acid sequence that encodes an amino acid that comprises 100% sequence identity to SEQ ID NO:36.

Embodiments of the disclosure include compositions, pharmaceutical compostions and cells of the disclosure comprising an expressible nucleic acid sequence that encode a plurality of tumor-specific antigens, wherein the expressible nucleic acid sequence comprises from about 15 to about 40 nucleotides that encode a portion of a tumor-specific antigens. The expressible nucleic acid may comprises any 15 to 40 contiguous nucleic acids presented below to encode a fragment of the amino acid sequence (or epitope). In some embodiments, the nucleic acid sequence comprise one or more epitopes chosen from about 30 to about 35 nucleotides of any of the sequences of Table 2.

TABLE 2
Full-length Wild-Type Sequences of Molecules in the Wnt Pathway

| SEQ | Name | | SEQ | Name | |
|---|---|---|---|---|---|
| ID | WT Wnt | | ID | WT Wnt | |
| NO | DNA | DNA Sequence | NO | mRNA | mRNA Sequence |
| 1 | WT beta- | ATGGCTACTCAAGCTGATTT | 2 | WT beta- | AUGGCUACUCAAGCUGAUUU |
| | catenin | GATGGAGTTGGACATGGCC | | catenin | GAUGGAGUUGGACAUGGCCA |
| | DNA | ATGGAACCAGACAGAAAAG | | mRNA | UGGAACCAGACAGAAAAGCG |
| | | CGGCTGTTAGTCACTGGCAG | | | GCUGUUAGUCACUGGCAGCA |
| | | CAACAGTCTTACCTGGACTC | | | ACAGUCUUACCUGGACUCUG |
| | | TGGAATCCATTCTGGTGCCA | | | GAAUCCAUUCUGGUGCCACU |
| | | CTACCACAGCTCCTTCTCTG | | | ACCACAGCUCCUUCUCUGAG |
| | | AGTGGTAAAGGCAATCCTG | | | UGGUAAAGGCAAUCCUGAGG |
| | | AGGAAGAGGATGTGGATAC | | | AAGAGGAUGUGGAUACCUCC |
| | | CTCCCAAGTCCTGTATGAGT | | | CAAGUCCUGUAUGAGUGGGA |
| | | GGGAACAGGGATTTTCTCA | | | ACAGGGAUUUUCUCAGUCCU |
| | | GTCCTTCACTCAAGAACAAG | | | UCACUCAAGAACAAGUAGCU |
| | | TAGCTGATATTGATGGACAG | | | GAUAUUGAUGGACAGUAUGC |
| | | TATGCAATGACTCGAGCTCA | | | AAUGACUCGAGCUCAGAGGG |
| | | GAGGGTACGAGCTGCTATG | | | UACGAGCUGCUAUGUUCCCU |
| | | TTCCCTGAGACATTAGATGA | | | GAGACAUUAGAUGAGGGCAU |
| | | GGGCATGCAGATCCCATCTA | | | GCAGAUCCCAUCUACACAGU |
| | | CACAGTTTGATGCTGCTCAT | | | UUGAUGCUGCUCAUCCCACU |
| | | CCCACTAATGTCCAGCGTTT | | | AAUGUCCAGCGUUUGGCUGA |
| | | GGCTGAACCATCACAGATG | | | ACCAUCACAGAUGCUGAAAC |
| | | CTGAAACATGCAGTTGTAA | | | AUGCAGUUGUAAACUUGAUU |
| | | ACTTGATTAACTATCAAGAT | | | AACUAUCAAGAUGAUGCAGA |
| | | GATGCAGAACTTGCCACAC | | | ACUUGCCACACGUGCAAUCC |
| | | GTGCAATCCCTGAACTGACA | | | CUGAACUGACAAAACUGCUA |
| | | AAACTGCTAAATGACGAGG | | | AAUGACGAGGACCAGGUGGU |
| | | ACCAGGTGGTGGTTAATAA | | | GGUUAAUAAGGCUGCAGUUA |
| | | GGCTGCAGTTATGGTCCATC | | | UGGUCCAUCAGCUUUCUAAA |
| | | AGCTTTCTAAAAAGGAAGC | | | AAGGAAGCUUCCAGACACGC |
| | | TTCCAGACACGCTATCATGC | | | UAUCAUGCGUUCUCCUCAGA |
| | | GTTCTCCTCAGATGGTGTCT | | | UGGUGUCUGCUAUUGUACGU |
| | | GCTATTGTACGTACCATGCA | | | ACCAUGCAGAAUACAAAUGA |
| | | GAATACAAATGATGTAGAA | | | UGUAGAAACAGCUCGUUGUA |
| | | ACAGCTCGTTGTACCGCTGG | | | CCGCUGGGACCUUGCAUAAC |
| | | GACCTTGCATAACCTTTCCC | | | CUUUCCCAUCAUCGUGAGGG |
| | | ATCATCGTGAGGGCTTACTG | | | CUUACUGGCCAUCUUUAAGU |
| | | GCCATCTTTAAGTCTGGAGG | | | CUGGAGGCAUUCCUGCCCUG |
| | | CATTCCTGCCCTGGTGAAAA | | | GUGAAAAUGCUUGGUUCACC |
| | | TGCTTGGTTCACCAGTGGAT | | | AGUGGAUUCUGUGUUGUUUU |
| | | TCTGTGTTGTTTTATGCCATT | | | AUGCCAUUACAACUCUCCAC |
| | | ACAACTCTCCACAACCTTTT | | | AACCUUUUAUUACAUCAAGA |
| | | ATTACATCAAGAAGGAGCT | | | AGGAGCUAAAAUGGCAGUGC |
| | | AAAATGGCAGTGCGTTTAG | | | GUUUAGCUGGUGGGCUGCAG |
| | | CTGGTGGGCTGCAGAAAAT | | | AAAAUGGUUGCCUUGCUCAA |
| | | GGTTGCCTTGCTCAACAAAA | | | CAAAACAAAUGUUAAAUUCU |
| | | CAAATGTTAAATTCTTGGCT | | | UGGCUAUUACGACAGACUGC |
| | | ATTACGACAGACTGCCTTCA | | | CUUCAAAUUUUAGCUUAUGG |
| | | AATTTTAGCTTATGGCAACC | | | CAACCAAGAAAGCAAGCUCA |
| | | AAGAAAGCAAGCTCATCAT | | | UCAUACUGGCUAGUGGUGGA |
| | | ACTGGCTAGTGGTGGACCCC | | | CCCCAAGCUUUAGUAAAUAU |
| | | AAGCTTTAGTAAATATAATG | | | AAUGAGGACCUAUACUUACG |
| | | AGGACCTATACTTACGAAA | | | AAAAACUACUGUGGACCACA |
| | | AACTACTGTGGACCACAAG | | | AGCAGAGUGCUGAAGGUGCU |
| | | CAGAGTGCTGAAGGTGCTA | | | AUCUGUCUGCUCUAGUAAUA |
| | | TCTGTCTGCTCTAGTAATAA | | | AGCCGGCUAUUGUAGAAGCU |
| | | GCCGGCTATTGTAGAAGCTG | | | GGUGGAAUGCAAGCUUUAGG |
| | | GTGGAATGCAAGCTTTAGG | | | ACUUCACCUGACAGAUCCAA |
| | | ACTTCACCTGACAGATCCAA | | | GUCAACGUCUUGUUCAGAAC |
| | | GTCAACGTCTTGTTCAGAAC | | | UGUCUUUGGACUCUCAGGAA |
| | | TGTCTTTGGACTCTCAGGAA | | | UCUUUCAGAUGCUGCAACUA |
| | | TCTTTCAGATGCTGCAACTA | | | AACAGGAAGGGAUGGAAGGU |
| | | AACAGGAAGGGATGGAAGG | | | CUCCUUGGGACUCUUGUUCA |
| | | TCTCCTTGGGACTCTTGTTC | | | GCUUCUGGGUUCAGAUGAUA |
| | | AGCTTCTGGGTTCAGATGAT | | | UAAAUGUGGUCACCUGUGCA |
| | | ATAAATGTGGTCACCTGTGC | | | GCUGGAAUUCUUUCUAACCU |
| | | AGCTGGAATTCTTTCTAACC | | | CACUUGCAAUAAUUAUAAGA |
| | | TCACTTGCAATAATTATAAG | | | ACAAGAUGAUGGUCUGCCAA |
| | | AACAAGATGATGGTCTGCC | | | GUGGGUGGUAUAGAGGCUCU |
| | | AAGTGGGTGGTATAGAGGC | | | UGUGCGUACUGUCCUUCGGG |
| | | TCTTGTGCGTACTGTCCTTC | | | CUGGUGACAGGGAAGACAUC |
| | | GGGCTGGTGACAGGGAAGA | | | ACUGAGCCUGCCAUCUGUGC |
| | | CATCACTGAGCCTGCCATCT | | | UCUUCGUCAUCUGACCAGCC |
| | | GTGCTCTTCGTCATCTGACC | | | GACACCAAGAAGCAGAGAUG |
| | | AGCCGACACCAAGAAGCAG | | | GCCCAGAAUGCAGUUCGCCU |
| | | AGATGGCCCAGAATGCAGT | | | UCACUAUGGACUACCAGUUG |
| | | TCGCCTTCACTATGGACTAC | | | UGGUUAAGCUCUUACACCCA |
| | | CAGTTGTGGTTAAGCTCTTA | | | CCAUCCCACUGGCCUCUGAU |
| | | CACCCACCATCCCACTGGCC | | | AAAGGCUACUGUUGGAUUGA |
| | | TCTGATAAAGGCTACTGTTG | | | UUCGAAAUCUUGCCCUUUGU |
| | | GATTGATTCGAAATCTTGCC | | | CCCGCAAAUCAUGCACCUUU |
| | | CTTTGTCCCGCAAATCATGC | | | GCGUGAGCAGGGUGCCAUUC |
| | | ACCTTTGCGTGAGCAGGGTG | | | CACGACUAGUUCAGUUGCUU |
| | | CCATTCCACGACTAGTTCAG | | | GUUCGUGCACAUCAGGAUAC |
| | | TTGCTTGTTCGTGCACATCA | | | CCAGCGCCGUACGUCCAUGG |
| | | GGATACCCAGCGCCGTACG | | | GUGGGACACAGCAGCAAUUU |
| | | TCCATGGGTGGGACACAGC | | | GUGGAGGGGGUCCGCAUGGA |
| | | AGCAATTTGTGGAGGGGGT | | | AGAAAUAGUUGAAGGUUGUA |
| | | CCGCATGGAAGAAATAGTT | | | CCGGAGCCCUUCACAUCCUA |
| | | GAAGGTTGTACCGGAGCCC | | | GCUCGGGAUGUUCACAACCG |
| | | TTCACATCCTAGCTCGGGAT | | | AAUUGUUAUCAGAGGACUAA |
| | | GTTCACAACCGAATTGTTAT | | | AUACCAUUCCAUUGUUUGUG |
| | | CAGAGGACTAAATACCATT | | | CAGCUGCUUUAUUCUCCCAU |
| | | CCATTGTTTGTGCAGCTGCT | | | UGAAAACAUCCAAAGAGUAG |
| | | TTATTCTCCCATTGAAAACA | | | CUGCAGGGGUCCUCUGUGAA |
| | | TCCAAAGAGTAGCTGCAGG | | | CUUGCUCAGGACAAGGAAGC |
| | | GGTCCTCTGTGAACTTGCTC | | | UGCAGAAGCUAUUGAAGCUG |
| | | AGGACAAGGAAGCTGCAGA | | | AGGGAGCCACAGCUCCUCUG |
| | | AGCTATTGAAGCTGAGGGA | | | ACAGAGUUACUUCACUCUAG |
| | | GCCACAGCTCCTCTGACAGA | | | GAAUGAAGGUGUGGCGACAU |
| | | GTTACTTCACTCTAGGAATG | | | AUGCAGCUGCUGUUUUGUUC |
| | | AAGGTGTGGCGACATATGC | | | CGAAUGUCUGAGGACAAGCC |
| | | AGCTGCTGTTTTGTTCCGAA | | | ACAAGAUUACAAGAAACGGC |
| | | TGTCTGAGGACAAGCCACA | | | UUUCAGUUGAGCUGACCAGC |
| | | AGATTACAAGAAACGGCTT | | | UCUCUCUUCAGAACAGAGCC |
| | | TCAGTTGAGCTGACCAGCTC | | | AAUGGCUUGGAAUGAGACUG |
| | | TCTCTTCAGAACAGAGCCAA | | | CUGAUCUUGGACUUGAUAUU |
| | | TGGCTTGGAATGAGACTGCT | | | GGUGCCCAGGGAGAACCCCU |
| | | GATCTTGGACTTGATATTGG | | | UGGAUAUCGCCAGGAUGAUC |
| | | TGCCCAGGGAGAACCCCTT | | | CUAGCUAUCGUUCUUUUCAC |
| | | GGATATCGCCAGGATGATC | | | UCUGGUGGAUAUGGCCAGGA |
| | | CTAGCTATCGTTCTTTTCAC | | | UGCCUUGGGUAUGGACCCCA |
| | | TCTGGTGGATATGGCCAGG | | | UGAUGGAACAUGAGAUGGGU |
| | | ATGCCTTGGGTATGGACCCC | | | GGCCACCACCCUGGUGCUGA |
| | | ATGATGGAACATGAGATGG | | | CUAUCCAGUUGAUGGGCUGC |
| | | GTGGCCACCACCCTGGTGCT | | | CAGAUCUGGGGCAUGCCCAG |
| | | GACTATCCAGTTGATGGGCT | | | GACCUCAUGGAUGGGCUGCC |
| | | GCCAGATCTGGGGCATGCC | | | UCCAGGUGACAGCAAUCAGC |
| | | CAGGACCTCATGGATGGGC | | | UGGCCUGGUUUGAUACUGAC |
| | | TGCCTCCAGGTGACAGCAAT | | | CUGUAA |
| | | CAGCTGGCCTGGTTTGATAC | | | |
| | | TGACCTGTAA | | | |
| 3 | WT | ATGAATATCCAAGAGCAGG | 4 | WT | AUGAAUAUCCAAGAGCAGGG |
| | AXIN1 | GTTTCCCCTTGGACCTCGGA | | AXIN1 | UUUCCCCUUGGACCUCGGAG |
| | DNA | GCAAGTTTCACCGAAGATG | | mRNA | CAAGUUUCACCGAAGAUGCU |
| | | CTCCCCGACCCCCAGTGCCT | | | CCCCGACCCCCAGUGCCUGGU |
| | | GGTGAGGAGGGAGAACTGG | | | GAGGAGGGAGAACUGGUGUC |
| | | TGTCCACAGACCCGAGGCC | | | CACAGACCCGAGGCCCGCCAG |
| | | CGCCAGCTACAGTTTCTGCT | | | CUACAGUUUCUGCUCCGGGA |
| | | CCGGGAAAGGTGTTGGCAT | | | AAGGUGUUGGCAUUAAAGGU |
| | | TAAAGGTGAGACTTCGACG | | | GAGACUUCGACGGCCACUCC |
| | | GCCACTCCGAGGCGCTCGG | | | GAGGCGCUCGGAUCUGGACC |
| | | ATCTGGACCTGGGGTATGA | | | UGGGGUAUGAGCCUGAGGGC |
| | | GCCTGAGGGCAGTGCCTCCC | | | AGUGCCUCCCCCACCCCACCA |
| | | CCACCCCACCATACTTGAAG | | | UACUUGAAGUGGGCUGAGUC |
| | | TGGGCTGAGTCACTGCATTC | | | ACUGCAUUCCCUGCUGGAUG |
| | | CCTGCTGGATGACCAAGAT | | | ACCAAGAUGGGAUAAGCCUG |
| | | GGGATAAGCCTGTTCAGGA | | | UUCAGGACUUUCCUGAAGCA |
| | | CTTTCCTGAAGCAGGAGGG | | | GGAGGGCUGUGCCGACUUGC |
| | | CTGTGCCGACTTGCTGGACT | | | UGGACUUCUGGUUUGCCUGC |
| | | TCTGGTTTGCCTGCACTGGC | | | ACUGGCUUCAGGAAGCUGGA |
| | | TTCAGGAAGCTGGAGCCCT | | | GCCCUGUGACUCGAACGAGG |
| | | GTGACTCGAACGAGGAGAA | | | AGAAGAGGCUGAAGCUGGCG |
| | | GAGGCTGAAGCTGGCGAGA | | | AGAGCCAUCUACCGAAAGUA |
| | | GCCATCTACCGAAAGTACAT | | | CAUUCUUGAUAACAAUGGCA |
| | | TCTTGATAACAATGGCATCG | | | UCGUGUCCCGGCAGACCAAG |
| | | TGTCCCGGCAGACCAAGCC | | | CCAGCCACCAAGAGCUUCAU |
| | | AGCCACCAAGAGCTTCATA | | | AAAGGGCUGCAUCAUGAAGC |
| | | AAGGGCTGCATCATGAAGC | | | AGCUGAUCGAUCCUGCCAUG |
| | | AGCTGATCGATCCTGCCATG | | | UUUGACCAGGCCCAGACCGA |
| | | TTTGACCAGGCCCAGACCG | | | AAUCCAGGCCACUAUGGAGG |
| | | AAATCCAGGCCACTATGGA | | | AAAACACCUAUCCCUCCUUCC |
| | | GGAAAACACCTATCCCTCCT | | | UUAAGUCUGAUAUUUAUUUG |
| | | TCCTTAAGTCTGATATTTAT | | | GAAUAUACGAGGACAGGCUC |
| | | TTGGAATATACGAGGACAG | | | GGAGAGCCCCAAAGUCUGUA |
| | | GCTCGGAGAGCCCCAAAGT | | | GUGACCAGAGCUCUGGGUCA |
| | | CTGTAGTGACCAGAGCTCTG | | | GGGACAGGGAAGGGCAUAUC |
| | | GGTCAGGGACAGGGAAGGG | | | UGGAUACCUGCCGACCUUAA |
| | | CATATCTGGATACCTGCCGA | | | AUGAAGAUGAGGAAUGGAAG |
| | | CCTTAAATGAAGATGAGGA | | | UGUGACCAGGACAUGGAUGA |
| | | ATGGAAGTGTGACCAGGAC | | | GGACGAUGGCAGAGACGCUG |
| | | ATGGATGAGGACGATGGCA | | | CUCCCCCCGGAAGACUCCCUC |
| | | GAGACGCTGCTCCCCCCGG | | | AGAAGCUGCUCCUGGAGACA |
| | | AAGACTCCCTCAGAAGCTG | | | GCUGCCCCGAGGGUCUCCUCC |
| | | CTCCTGGAGACAGCTGCCCC | | | AGUAGACGGUACAGCGAAGG |
| | | GAGGGTCTCCTCCAGTAGAC | | | CAGAGAGUUCAGGUAUGGAU |
| | | GGTACAGCGAAGGCAGAGA | | | CCUGGCGGGAGCCAGUCAAC |
| | | GTTCAGGTATGGATCCTGGC | | | CCCUAUUAUGUCAAUGCCGG |
| | | GGGAGCCAGTCAACCCCTA | | | CUAUGCCCUGGCCCCAGCCAC |
| | | TTATGTCAATGCCGGCTATG | | | CAGUGCCAACGACAGCGAGC |
| | | CCCTGGCCCCAGCCACCAGT | | | AGCAGAGCCUGUCCAGCGAU |
| | | GCCAACGACAGCGAGCAGC | | | GCAGACACCCUGUCCCUCACG |
| | | AGAGCCTGTCCAGCGATGC | | | GACAGCAGCGUGGAUGGGAU |
| | | AGACACCCTGTCCCTCACGG | | | CCCCCCAUACAGGAUCCGUA |
| | | ACAGCAGCGTGGATGGGAT | | | AGCAGCACCGCAGGGAGAUG |
| | | CCCCCCATACAGGATCCGTA | | | CAGGAGAGCGUGCAGGUCAA |
| | | AGCAGCACCGCAGGGAGAT | | | UGGGCGGGUGCCCCUACCUC |
| | | GCAGGAGAGCGTGCAGGTC | | | ACAUUCCCCGCACGUACCGG |
| | | AATGGGCGGGTGCCCCTAC | | | GUGCCGAAGGAGGUCCGCGU |
| | | CTCACATTCCCCGCACGTAC | | | GGAGCCUCAGAAGUUCGCGG |
| | | CGGGTGCCGAAGGAGGTCC | | | AGGAGCUCAUCCACCGCCUG |
| | | GCGTGGAGCCTCAGAAGTT | | | GAGGCUGUGCAGCGCACGCG |
| | | CGCGGAGGAGCTCATCCAC | | | GGAGGCCGAGGAGAAGCUGG |
| | | CGCCTGGAGGCTGTGCAGC | | | AGGAGCGGCUGAAGCGCGUG |
| | | GCACGCGGGAGGCCGAGGA | | | CGCAUGGAGGAGGAAGGUGA |
| | | GAAGCTGGAGGAGCGGCTG | | | GGACGGCGAUCCAUCGUCAG |
| | | AAGCGCGTGCGCATGGAGG | | | GGCCCCCAGGGCCGUGUCAC |
| | | AGGAAGGTGAGGACGGCGA | | | AAGCUGCCUCCCGCCCCCGCU |
| | | TCCATCGTCAGGGCCCCCAG | | | UGGCACCACUUCCCGCCCCGC |
| | | GGCCGTGTCACAAGCTGCCT | | | UGUGUGGACAUGGGCUGUGC |
| | | CCCGCCCCCGCTTGGCACCA | | | CGGGCUCCGGGAUGCACACG |
| | | CTTCCCGCCCCGCTGTGTGG | | | AGGAGAACCCUGAGAGCAUC |
| | | ACATGGGCTGTGCCGGGCTC | | | CUGGACGAGCACGUACAGCG |
| | | CGGGATGCACACGAGGAGA | | | UGUGCUGAGGACACCUGGCC |
| | | ACCCTGAGAGCATCCTGGA | | | GCCAGUCGCCUGGGCCUGGC |
| | | CGAGCACGTACAGCGTGTG | | | CAUCGCUCCCCGGACAGUGG |
| | | CTGAGGACACCTGGCCGCC | | | GCACGUGGCCAAGAUGCCAG |
| | | AGTCGCCTGGGCCTGGCCAT | | | UGGCACUGGGGGGUGCCGCC |
| | | CGCTCCCCGGACAGTGGGC | | | UCGGGGCACGGGAAGCACGU |
| | | ACGTGGCCAAGATGCCAGT | | | ACCCAAGUCAGGGGCGAAGC |
| | | GGCACTGGGGGGTGCCGCC | | | UGGACGCGGCCGGCCUGCAC |
| | | TCGGGGCACGGGAAGCACG | | | CACCACCGACACGUCCACCAC |
| | | TACCCAAGTCAGGGGCGAA | | | CACGUCCACCACAGCACAGCC |
| | | GCTGGACGCGGCCGGCCTG | | | CGGCCCAAGGAGCAGGUGGA |
| | | CACCACCACCGACACGTCC | | | GGCCGAGGCCACCCGCAGGG |
| | | ACCACCACGTCCACCACAG | | | CCCAGAGCAGCUUCGCCUGG |
| | | CACAGCCCGGCCCAAGGAG | | | GGCCUGGAACCACACAGCCA |
| | | CAGGTGGAGGCCGAGGCCA | | | UGGGGCAAGGUCCCGAGGCU |
| | | CCCGCAGGGCCCAGAGCAG | | | ACUCAGAGAGUGUUGGCGCU |
| | | CTTCGCCTGGGGCCTGGAAC | | | GCCCCCAACGCCAGUGAUGG |
| | | CACACAGCCATGGGGCAAG | | | CCUCGCCCACAGUGGGAAGG |
| | | GTCCCGAGGCTACTCAGAG | | | UGGGCGUGGCGUGCAAAAGA |
| | | AGTGTTGGCGCTGCCCCCAA | | | AAUGCCAAGAAGGCUGAGUC |
| | | CGCCAGTGATGGCCTCGCCC | | | GGGGAAGAGCGCCAGCACCG |
| | | ACAGTGGGAAGGTGGGCGT | | | AGGUGCCAGGUGCCUCGGAG |
| | | GGCGTGCAAAAGAAATGCC | | | GAUGCGGAGAAGAACCAGAA |
| | | AAGAAGGCTGAGTCGGGGA | | | AAUCAUGCAGUGGAUCAUUG |
| | | AGAGCGCCAGCACCGAGGT | | | AGGGGGAAAAGGAGAUCAGC |
| | | GCCAGGTGCCTCGGAGGAT | | | AGGCACCGCAGGACCGGCCA |
| | | GCGGAGAAGAACCAGAAAA | | | CGGGUCUUCGGGGACGAGGA |
| | | TCATGCAGTGGATCATTGAG | | | AGCCACAGCCCCAUGAGAAC |
| | | GGGGAAAAGGAGATCAGCA | | | UCCAGACCCUUGUCCCUUGA |
| | | GGCACCGCAGGACCGGCCA | | | GCACCCCUGGGCCGGCCCUCA |
| | | CGGGTCTTCGGGGACGAGG | | | GCUCCGGACCUCCGUGCAGCC |
| | | AAGCCACAGCCCCATGAGA | | | CUCCCACCUCUUCAUCCAAGA |
| | | ACTCCAGACCCTTGTCCCTT | | | CCCCACCAUGCCACCCCACCC |
| | | GAGCACCCCTGGGCCGGCC | | | AGCUCCCAACCCCCUAACCCA |
| | | CTCAGCTCCGGACCTCCGTG | | | GCUGGAGGAGGCGCGCCGAC |
| | | CAGCCCTCCCACCTCTTCAT | | | GUCUGGAGGAGGAAGAAAAG |
| | | CCAAGACCCCACCATGCCA | | | AGAGCCAGCCGAGCACCCUCC |
| | | CCCCACCCAGCTCCCAACCC | | | AAGCAGAGGUAUGUGCAGGA |
| | | CCTAACCCAGCTGGAGGAG | | | GGUUAUGCGGCGGGGACGCG |
| | | GCGCGCCGACGTCTGGAGG | | | CCUGCGUCAGGCCAGCGUGC |
| | | AGGAAGAAAAGAGAGCCAG | | | GCGCCGGUGCUGCACGUGGU |
| | | CCGAGCACCCTCCAAGCAG | | | ACCAGCCGUGUCGGACAUGG |
| | | AGGTATGTGCAGGAGGTTA | | | AGCUCUCCGAGACAGAGACA |
| | | TGCGGCGGGGACGCGCCTG | | | AGAUCGCAGAGGAAGGUGGG |
| | | CGTCAGGCCAGCGTGCGCG | | | CGGCGGGAGUGCCCAGCCGU |
| | | CCGGTGCTGCACGTGGTACC | | | GUGACAGCAUCGUUGUGGCG |
| | | AGCCGTGTCGGACATGGAG | | | UACUACUUCUGCGGGGAACC |
| | | CTCTCCGAGACAGAGACAA | | | CAUCCCCUACCGCACCCUGGU |
| | | GATCGCAGAGGAAGGTGGG | | | GAGGGGCCGCGCUGUCACCC |
| | | CGGCGGGAGTGCCCAGCCG | | | UGGGCCAGUUCAAGGAGCUG |
| | | TGTGACAGCATCGTTGTGGC | | | CUGACCAAAAAGGGCAGCUA |
| | | GTACTACTTCTGCGGGGAAC | | | CAGAUACUACUUCAAGAAAG |
| | | CCATCCCCTACCGCACCCTG | | | UGAGCGACGAGUUUGACUGU |
| | | GTGAGGGGCCGCGCTGTCA | | | GGGGUGGUGUUUGAGGAGGU |
| | | CCCTGGGCCAGTTCAAGGA | | | UCGAGAGGACGAGGCCGUCC |
| | | GCTGCTGACCAAAAAGGGC | | | UGCCCGUCUUUGAGGAGAAG |
| | | AGCTACAGATACTACTTCAA | | | AUCAUCGGCAAAGUGGAGAA |
| | | GAAAGTGAGCGACGAGTTT | | | GGUGGACUGA |
| | | GACTGTGGGGTGGTGTTTGA | | | |
| | | GGAGGTTCGAGAGGACGAG | | | |
| | | GCCGTCCTGCCCGTCTTTGA | | | |
| | | GGAGAAGATCATCGGCAAA | | | |
| | | GTGGAGAAGGTGGACTGA | | | |
| 5 | WT | ATGAGTAGCGCTATGTTGGT | 6 | WT | AUGAGUAGCGCUAUGUUGGU |
| | AXIN2 | GACTTGCCTCCCGGACCCCA | | AXIN2 | GACUUGCCUCCCGGACCCCAG |
| | DNA | GCAGCAGCTTCCGTGAGGA | | RNA | CAGCAGCUUCCGUGAGGAUG |
| | | TGCCCCGCGGCCCCCAGTGC | | | CCCCGCGGCCCCCAGUGCCAG |
| | | CAGGGGAAGAAGGGGAGAC | | | GGGAAGAAGGGGAGACCCCA |
| | | CCCACCGTGTCAGCCAGGG | | | CCGUGUCAGCCAGGGGUGGG |
| | | GTGGGCAAGGGCCAGGTCA | | | CAAGGGCCAGGUCACCAAAC |
| | | CCAAACCCATGCCTGTCTCT | | | CCAUGCCUGUCUCUUCCAAC |
| | | TCCAACACCAGGCGGAACG | | | ACCAGGCGGAACGAAGAUGG |
| | | AAGATGGGTTGGGGGAGCC | | | GUUGGGGGAGCCGGAGGGGC |
| | | GGAGGGGCGGGCATCTCCG | | | GGGCAUCUCCGGAUUCCCCU |
| | | GATTCCCCTCTGACCCGGTG | | | CUGACCCGGUGGACCAAGUC |
| | | GACCAAGTCCTTACACTCCT | | | CUUACACUCCUUAUUGGGCG |
| | | TATTGGGCGATCAAGACGG | | | AUCAAGACGGUGCUUACCUG |
| | | TGCTTACCTGTTCCGAACTT | | | UUCCGAACUUUCCUGGAGAG |
| | | TCCTGGAGAGGGAGAAATG | | | GGAGAAAUGCGUGGAUACCU |
| | | CGTGGATACCTTAGACTTCT | | | UAGACUUCUGGUUUGCCUGC |
| | | GGTTTGCCTGCAATGGATTC | | | AAUGGAUUCAGGCAGAUGAA |
| | | AGGCAGATGAACCTGAAGG | | | CCUGAAGGAUACCAAAACUU |
| | | ATACCAAAACTTTACGAGTA | | | UACGAGUAGCCAAAGCGAUC |
| | | GCCAAAGCGATCTACAAAA | | | UACAAAAGGUACAUUGAGAA |
| | | GGTACATTGAGAACAACAG | | | CAACAGCAUUGUCUCCAAGC |
| | | CATTGTCTCCAAGCAGCTGA | | | AGCUGAAGCCUGCCACCAAG |
| | | AGCCTGCCACCAAGACCTA | | | ACCUACAUAAGAGAUGGCAU |
| | | CATAAGAGATGGCATCAAG | | | CAAGAAGCAGCAGAUUGAUU |
| | | AAGCAGCAGATTGATTCCAT | | | CCAUCAUGUUUGACCAGGCG |
| | | CATGTTTGACCAGGCGCAG | | | CAGACCGAGAUCCAGUCGGU |
| | | ACCGAGATCCAGTCGGTGA | | | GAUGGAGGAAAAUGCCUACC |
| | | TGGAGGAAAATGCCTACCA | | | AGAUGUUUUUGACUUCUGAU |
| | | GATGTTTTTGACTTCTGATA | | | AUAUACCUCGAAUAUGUGAG |
| | | TATACCTCGAATATGTGAGG | | | GAGUGGGGGAGAAAACACAG |
| | | AGTGGGGGAGAAAACACAG | | | CUUACAUGAGUAAUGGGGGA |
| | | CTTACATGAGTAATGGGGG | | | CUCGGGAGCCUAAAGGUCGU |
| | | ACTCGGGAGCCTAAAGGTC | | | GUGUGGCUAUCUCCCCACCU |
| | | GTGTGTGGCTATCTCCCCAC | | | UGAAUGAAGAAGAGGAGUGG |
| | | CTTGAATGAAGAAGAGGAG | | | ACUUGUGCCGACUUCAAGUG |
| | | TGGACTTGTGCCGACTTCAA | | | CAAACUUUCGCCAACCGUGG |
| | | GTGCAAACTTTCGCCAACCG | | | UUGGCUUGUCCAGCAAAACU |
| | | TGGTTGGCTTGTCCAGCAAA | | | CUGAGGGCCACGGCGAGUGU |
| | | ACTCTGAGGGCCACGGCGA | | | GAGGUCCACGGAAACUGUUG |
| | | GTGTGAGGTCCACGGAAAC | | | ACAGUGGAUACAGGUCCUUC |
| | | TGTTGACAGTGGATACAGGT | | | AAGAGGAGCGAUCCUGUUAA |
| | | CCTTCAAGAGGAGCGATCCT | | | UCCUUAUCACAUAGGUUCUG |
| | | GTTAATCCTTATCACATAGG | | | GCUAUGUCUUUGCACCAGCC |
| | | TTCTGGCTATGTCTTTGCAC | | | ACCAGCGCCAACGACAGUGA |
| | | CAGCCACCAGCGCCAACGA | | | GAUAUCCAGUGAUGCGCUGA |
| | | CAGTGAGATATCCAGTGAT | | | CGGAUGAUUCCAUGUCCAUG |
| | | GCGCTGACGGATGATTCCAT | | | ACGGACAGCAGUGUAGAUGG |
| | | GTCCATGACGGACAGCAGT | | | AAUUCCUCCUUAUCGUGUGG |
| | | GTAGATGGAATTCCTCCTTA | | | GCAGUAAGAAACAGCUCCAG |
| | | TCGTGTGGGCAGTAAGAAA | | | AGAGAAAUGCAUCGCAGUGU |
| | | CAGCTCCAGAGAGAAATGC | | | GAAGGCCAAUGGCCAAGUGU |
| | | ATCGCAGTGTGAAGGCCAA | | | CUCUACCUCAUUUCCCGAGA |
| | | TGGCCAAGTGTCTCTACCTC | | | ACCCACCGCCUGCCCAAGGAG |
| | | ATTTCCCGAGAACCCACCGC | | | AUGACCCCCGUGGAACCCGCC |
| | | CTGCCCAAGGAGATGACCC | | | ACCUUUGCAGCUGAGCUGAU |
| | | CCGTGGAACCCGCCACCTTT | | | CUCGAGGCUGGAAAAGCUGA |
| | | GCAGCTGAGCTGATCTCGA | | | AGCUGGAGUUGGAGAGCCGC |
| | | GGCTGGAAAAGCTGAAGCT | | | CACAGCCUGGAGGAGCGCCU |
| | | GGAGTTGGAGAGCCGCCAC | | | GCAGCAGAUCCGAGAGGAUG |
| | | AGCCTGGAGGAGCGCCTGC | | | AAGAGAGAGAGGGCUCCGAG |
| | | AGCAGATCCGAGAGGATGA | | | CUCACACUCAAUUCGCGGGA |
| | | AGAGAGAGAGGGCTCCGAG | | | GGGGGCGCCCACGCAGCACCC |
| | | CTCACACTCAATTCGCGGGA | | | CCUCUCCCUACUGCCCUCCGG |
| | | GGGGGCGCCCACGCAGCAC | | | CAGCUACGAGGAAGACCCGC |
| | | CCCCTCTCCCTACTGCCCTC | | | AGACGAUACUGGACGAUCAC |
| | | CGGCAGCTACGAGGAAGAC | | | CUGUCCAGGGUCCUCAAGAC |
| | | CCGCAGACGATACTGGACG | | | CCCUGGCUGCCAGUCUCCAG |
| | | ATCACCTGTCCAGGGTCCTC | | | GCGUAGGCCGCUAUAGCCCC |
| | | AAGACCCCTGGCTGCCAGTC | | | CGCUCCCGCUCCCCGGACCAC |
| | | TCCAGGCGTAGGCCGCTATA | | | CACCACCACCACCAUUCGCAG |
| | | GCCCCCGCTCCCGCTCCCCG | | | UACCACUCCCUGCUCCCGCCC |
| | | GACCACCACCACCACCACC | | | GGUGGCAAGCUGCCUCCCGC |
| | | ATTCGCAGTACCACTCCCTG | | | GGCCGCCUCGCCGGGCGCCUG |
| | | CTCCCGCCCGGTGGCAAGCT | | | CCCCCUCCUCGGGGGCAAAG |
| | | GCCTCCCGCGGCCGCCTCGC | | | GCUUUGUGACCAAGCAGACG |
| | | CGGGCGCCTGCCCCCTCCTC | | | ACGAAGCAUGUCCACCACCA |
| | | GGGGGCAAAGGCTTTGTGA | | | CUACAUCCACCACCAUGCCGU |
| | | CCAAGCAGACGACGAAGCA | | | CCCCAAGACCAAGGAGGAGA |
| | | TGTCCACCACCACTACATCC | | | UCGAGGCGGAGGCCACGCAG |
| | | ACCACCATGCCGTCCCCAAG | | | CGGGUGCACUGCUUCUGCCC |
| | | ACCAAGGAGGAGATCGAGG | | | UGGGGGCAGCGAGUAUUACU |
| | | CGGAGGCCACGCAGCGGGT | | | GCUACUCGAAAUGCAAAAGC |
| | | GCACTGCTTCTGCCCTGGGG | | | CACUCCAAGGCUCCGGAAAC |
| | | GCAGCGAGTATTACTGCTAC | | | CAUGCCCAGCGAGCAGUUUG |
| | | TCGAAATGCAAAAGCCACT | | | GCGGCAGCAGAGGCAGUACC |
| | | CCAAGGCTCCGGAAACCAT | | | UUGCCCAAACGCAAUGGGAA |
| | | GCCCAGCGAGCAGTTTGGC | | | AGGCACGGAGCCGGGCCUGG |
| | | GGCAGCAGAGGCAGTACCT | | | CCCUGCCCGCCAGGGAAGGA |
| | | TGCCCAAACGCAATGGGAA | | | GGGGCCCCCGGCGGAGCUGG |
| | | AGGCACGGAGCCGGGCCTG | | | GGCCCUGCAGCUUCCCCGGG |
| | | GCCCTGCCCGCCAGGGAAG | | | AGGAAGGAGACAGGUCGCAG |
| | | GAGGGGCCCCCGGCGGAGC | | | GAUGUCUGGCAGUGGAUGCU |
| | | TGGGGCCCTGCAGCTTCCCC | | | GGAGAGUGAGCGGCAGAGCA |
| | | GGGAGGAAGGAGACAGGTC | | | AGCCCAAGCCCCAUAGUGCCC |
| | | GCAGGATGTCTGGCAGTGG | | | AAAGCACAAAAAAGGCCUAC |
| | | ATGCTGGAGAGTGAGCGGC | | | CCCUUGGAGUCUGCCCGCUC |
| | | AGAGCAAGCCCAAGCCCCA | | | GUCUCCAGGCGAACGAGCCA |
| | | TAGTGCCCAAAGCACAAAA | | | GCCGGCACCAUCUGUGGGGG |
| | | AAGGCCTACCCCTTGGAGTC | | | GGCAACAGCGGGCACCCCCGC |
| | | TGCCCGCTCGTCTCCAGGCG | | | ACCACCCCCCGUGCCCACCUG |
| | | AACGAGCCAGCCGGCACCA | | | UUCACCCAGGACCCUGCGAU |
| | | TCTGTGGGGGGGCAACAGC | | | GCCUCCCCUGACCCCACCCAA |
| | | GGGCACCCCCGCACCACCC | | | CACGCUGGCUCAGCUGGAGG |
| | | CCCGTGCCCACCTGTTCACC | | | AGGCCUGUCGCAGGCUAGCU |
| | | CAGGACCCTGCGATGCCTCC | | | GAGGUGUCGAAGCCCCCAAA |
| | | CCTGACCCCACCCAACACGC | | | GCAGCGGUGCUGUGUGGCCA |
| | | TGGCTCAGCTGGAGGAGGC | | | GUCAGCAGAGGGACAGGAAU |
| | | CTGTCGCAGGCTAGCTGAG | | | CAUUCGGCCACUGUUCAGAC |
| | | GTGTCGAAGCCCCCAAAGC | | | GGGAGCCACACCCUUCUCCA |
| | | AGCGGTGCTGTGTGGCCAGT | | | AUCCAAGCCUGGCUCCAGAA |
| | | CAGCAGAGGGACAGGAATC | | | GAUCACAAAGAGCCAAAGAA |
| | | ATTCGGCCACTGTTCAGACG | | | ACUGGCAGGUGUCCACGCGC |
| | | GGAGCCACACCCTTCTCCAA | | | UCCAGGCCAGUGAGUUGGUU |
| | | TCCAAGCCTGGCTCCAGAA | | | GUCACUUACUUUUUCUGUGG |
| | | GATCACAAAGAGCCAAAGA | | | GGAAGAAAUUCCAUACCGGA |
| | | AACTGGCAGGTGTCCACGC | | | GGAUGCUGAAGGCUCAGAGC |
| | | GCTCCAGGCCAGTGAGTTG | | | UUGACCCUGGGCCACUUUAA |
| | | GTTGTCACTTACTTTTTCTGT | | | AGAGCAGCUCAGCAAAAAGG |
| | | GGGGAAGAAATTCCATACC | | | GAAAUUAUAGGUAUUACUUC |
| | | GGAGGATGCTGAAGGCTCA | | | AAAAAAGCAAGCGAUGAGUU |
| | | GAGCTTGACCCTGGGCCACT | | | UGCCUGUGGAGCGGUGUUUG |
| | | TTAAAGAGCAGCTCAGCAA | | | AGGAGAUCUGGGAGGAUGAG |
| | | AAAGGGAAATTATAGGTAT | | | ACGGUGCUCCCGAUGUAUGA |
| | | TACTTCAAAAAAGCAAGCG | | | AGGCCGGAUUCUGGGCAAAG |
| | | ATGAGTTTGCCTGTGGAGCG | | | UGGAGCGGAUCGAUUGA |
| | | GTGTTTGAGGAGATCTGGG | | | |
| | | AGGATGAGACGGTGCTCCC | | | |
| | | GATGTATGAAGGCCGGATT | | | |
| | | CTGGGCAAAGTGGAGCGGA | | | |
| | | TCGATTGA | | | |
| 7 | WT APC | ATGGCTGCAGCTTCATATGA | 8 | WT APC | AUGGCUGCAGCUUCAUAUGA |
| | DNA | TCAGTTGTTAAAGCAAGTTG | | RNA | UCAGUUGUUAAAGCAAGUUG |
| | | AGGCACTGAAGATGGAGAA | | | AGGCACUGAAGAUGGAGAAC |
| | | CTCAAATCTTCGACAAGAGC | | | UCAAAUCUUCGACAAGAGCU |
| | | TAGAAGATAATTCCAATCAT | | | AGAAGAUAAUUCCAAUCAUC |
| | | CTTACAAAACTGGAAACTG | | | UUACAAAACUGGAAACUGAG |
| | | AGGCATCTAATATGAAGGA | | | GCAUCUAAUAUGAAGGAAGU |
| | | AGTACTTAAACAACTACAA | | | ACUUAAACAACUACAAGGAA |
| | | GGAAGTATTGAAGATGAAG | | | GUAUUGAAGAUGAAGCUAUG |
| | | CTATGGCTTCTTCTGGACAG | | | GCUUCUUCUGGACAGAUUGA |
| | | ATTGATTTATTAGAGCGTCT | | | UUUAUUAGAGCGUCUUAAAG |
| | | TAAAGAGCTTAACTTAGATA | | | AGCUUAACUUAGAUAGCAGU |
| | | GCAGTAATTTCCCTGGAGTA | | | AAUUUCCCUGGAGUAAAACU |
| | | AAACTGCGGTCAAAAATGT | | | GCGGUCAAAAAUGUCCCUCC |
| | | CCCTCCGTTCTTATGGAAGC | | | GUUCUUAUGGAAGCCGGGAA |
| | | CGGGAAGGATCTGTATCAA | | | GGAUCUGUAUCAAGCCGUUC |
| | | GCCGTTCTGGAGAGTGCAGT | | | UGGAGAGUGCAGUCCUGUUC |
| | | CCTGTTCCTATGGGTTCATT | | | CUAUGGGUUCAUUUCCAAGA |
| | | TCCAAGAAGAGGGTTTGTA | | | AGAGGGUUUGUAAAUGGAAG |
| | | AATGGAAGCAGAGAAAGTA | | | CAGAGAAAGUACUGGAUAUU |
| | | CTGGATATTTAGAAGAACTT | | | UAGAAGAACUUGAGAAAGAG |
| | | GAGAAAGAGAGGTCATTGC | | | AGGUCAUUGCUUCUUGCUGA |
| | | TTCTTGCTGATCTTGACAAA | | | UCUUGACAAAGAAGAAAAGG |
| | | GAAGAAAAGGAAAAAGACT | | | AAAAAGACUGGUAUUACGCU |
| | | GGTATTACGCTCAACTTCAG | | | CAACUUCAGAAUCUCACUAA |
| | | AATCTCACTAAAAGAATAG | | | AAGAAUAGAUAGUCUUCCUU |
| | | ATAGTCTTCCTTTAACTGAA | | | UAACUGAAAAUUUUUCCUUA |
| | | AATTTTTCCTTACAAACAGA | | | CAAACAGAUAUGACCAGAAG |
| | | TATGACCAGAAGGCAATTG | | | GCAAUUGGAAUAUGAAGCAA |
| | | GAATATGAAGCAAGGCAAA | | | GGCAAAUCAGAGUUGCGAUG |
| | | TCAGAGTTGCGATGGAAGA | | | GAAGAACAACUAGGUACCUG |
| | | ACAACTAGGTACCTGCCAG | | | CCAGGAUAUGGAAAAACGAG |
| | | GATATGGAAAAACGAGCAC | | | CACAGCGAAGAAUAGCCAGA |
| | | AGCGAAGAATAGCCAGAAT | | | AUUCAGCAAAUCGAAAAGGA |
| | | TCAGCAAATCGAAAAGGAC | | | CAUACUUCGUAUACGACAGC |
| | | ATACTTCGTATACGACAGCT | | | UUUUACAGUCCCAAGCAACA |
| | | TTTACAGTCCCAAGCAACAG | | | GAAGCAGAGAGGUCAUCUCA |
| | | AAGCAGAGAGGTCATCTCA | | | GAACAAGCAUGAAACCGGCU |
| | | GAACAAGCATGAAACCGGC | | | CACAUGAUGCUGAGCGGCAG |
| | | TCACATGATGCTGAGCGGC | | | AAUGAAGGUCAAGGAGUGGG |
| | | AGAATGAAGGTCAAGGAGT | | | AGAAAUCAACAUGGCAACUU |
| | | GGGAGAAATCAACATGGCA | | | CUGGUAAUGGUCAGGGUUCA |
| | | ACTTCTGGTAATGGTCAGGG | | | ACUACACGAAUGGACCAUGA |
| | | TTCAACTACACGAATGGACC | | | AACAGCCAGUGUUUUGAGUU |
| | | ATGAAACAGCCAGTGTTTTG | | | CUAGUAGCACACACUCUGCA |
| | | AGTTCTAGTAGCACACACTC | | | CCUCGAAGGCUGACAAGUCA |
| | | TGCACCTCGAAGGCTGACA | | | UCUGGGAACCAAGGUGGAAA |
| | | AGTCATCTGGGAACCAAGG | | | UGGUGUAUUCAUUGUUGUCA |
| | | TGGAAATGGTGTATTCATTG | | | AUGCUUGGUACUCAUGAUAA |
| | | TTGTCAATGCTTGGTACTCA | | | GGAUGAUAUGUCGCGAACUU |
| | | TGATAAGGATGATATGTCGC | | | UGCUAGCUAUGUCUAGCUCC |
| | | GAACTTTGCTAGCTATGTCT | | | CAAGACAGCUGUAUAUCCAU |
| | | AGCTCCCAAGACAGCTGTAT | | | GCGACAGUCUGGAUGUCUUC |
| | | ATCCATGCGACAGTCTGGAT | | | CUCUCCUCAUCCAGCUUUUA |
| | | GTCTTCCTCTCCTCATCCAG | | | CAUGGCAAUGACAAAGACUC |
| | | CTTTTACATGGCAATGACAA | | | UGUAUUGUUGGGAAAUUCCC |
| | | AGACTCTGTATTGTTGGGAA | | | GGGGCAGUAAAGAGGCUCGG |
| | | ATTCCCGGGGCAGTAAAGA | | | GCCAGGGCCAGUGCAGCACU |
| | | GGCTCGGGCCAGGGCCAGT | | | CCACAACAUCAUUCACUCAC |
| | | GCAGCACTCCACAACATCAT | | | AGCCUGAUGACAAGAGAGGC |
| | | TCACTCACAGCCTGATGACA | | | AGGCGUGAAAUCCGAGUCCU |
| | | AGAGAGGCAGGCGTGAAAT | | | UCAUCUUUUGGAACAGAUAC |
| | | CCGAGTCCTTCATCTTTTGG | | | GCGCUUACUGUGAAACCUGU |
| | | AACAGATACGCGCTTACTGT | | | UGGGAGUGGCAGGAAGCUCA |
| | | GAAACCTGTTGGGAGTGGC | | | UGAACCAGGCAUGGACCAGG |
| | | AGGAAGCTCATGAACCAGG | | | ACAAAAAUCCAAUGCCAGCU |
| | | CATGGACCAGGACAAAAAT | | | CCUGUUGAACAUCAGAUCUG |
| | | CCAATGCCAGCTCCTGTTGA | | | UCCUGCUGUGUGUGUUCUAA |
| | | ACATCAGATCTGTCCTGCTG | | | UGAAACUUUCAUUUGAUGAA |
| | | TGTGTGTTCTAATGAAACTT | | | GAGCAUAGACAUGCAAUGAA |
| | | TCATTTGATGAAGAGCATAG | | | UGAACUAGGGGGACUACAGG |
| | | ACATGCAATGAATGAACTA | | | CCAUUGCAGAAUUAUUGCAA |
| | | GGGGGACTACAGGCCATTG | | | GUGGACUGUGAAAUGUAUGG |
| | | CAGAATTATTGCAAGTGGA | | | GCUUACUAAUGACCACUACA |
| | | CTGTGAAATGTATGGGCTTA | | | GUAUUACACUAAGACGAUAU |
| | | CTAATGACCACTACAGTATT | | | GCUGGAAUGGCUUUGACAAA |
| | | ACACTAAGACGATATGCTG | | | CUUGACUUUUGGAGAUGUAG |
| | | GAATGGCTTTGACAAACTTG | | | CCAACAAGGCUACGCUAUGC |
| | | ACTTTTGGAGATGTAGCCAA | | | UCUAUGAAAGGCUGCAUGAG |
| | | CAAGGCTACGCTATGCTCTA | | | AGCACUUGUGGCCCAACUAA |
| | | TGAAAGGCTGCATGAGAGC | | | AAUCUGAAAGUGAAGACUUA |
| | | ACTTGTGGCCCAACTAAAAT | | | CAGCAGGUUAUUGCGAGUGU |
| | | CTGAAAGTGAAGACTTACA | | | UUUGAGGAAUUUGUCUUGGC |
| | | GCAGGTTATTGCGAGTGTTT | | | GAGCAGAUGUAAAUAGUAAA |
| | | TGAGGAATTTGTCTTGGCGA | | | AAGACGUUGCGAGAAGUUGG |
| | | GCAGATGTAAATAGTAAAA | | | AAGUGUGAAAGCAUUGAUGG |
| | | AGACGTTGCGAGAAGTTGG | | | AAUGUGCUUUAGAAGUUAAA |
| | | AAGTGTGAAAGCATTGATG | | | AAGGAAUCAACCCUCAAAAG |
| | | GAATGTGCTTTAGAAGTTAA | | | CGUAUUGAGUGCCUUAUGGA |
| | | AAAGGAATCAACCCTCAAA | | | AUUUGUCAGCACAUUGCACU |
| | | AGCGTATTGAGTGCCTTATG | | | GAGAAUAAAGCUGAUAUAUG |
| | | GAATTTGTCAGCACATTGCA | | | UGCUGUAGAUGGUGCACUUG |
| | | CTGAGAATAAAGCTGATAT | | | CAUUUUUGGUUGGCACUCUU |
| | | ATGTGCTGTAGATGGTGCAC | | | ACUUACCGGAGCCAGACAAA |
| | | TTGCATTTTTGGTTGGCACT | | | CACUUUAGCCAUUAUUGAAA |
| | | CTTACTTACCGGAGCCAGAC | | | GUGGAGGUGGGAUAUUACGG |
| | | AAACACTTTAGCCATTATTG | | | AAUGUGUCCAGCUUGAUAGC |
| | | AAAGTGGAGGTGGGATATT | | | UACAAAUGAGGACCACAGGC |
| | | ACGGAATGTGTCCAGCTTGA | | | AAAUCCUAAGAGAGAACAAC |
| | | TAGCTACAAATGAGGACCA | | | UGUCUACAAACUUUAUUACA |
| | | CAGGCAAATCCTAAGAGAG | | | ACACUUAAAAUCUCAUAGUU |
| | | AACAACTGTCTACAAACTTT | | | UGACAAUAGUCAGUAAUGCA |
| | | ATTACAACACTTAAAATCTC | | | UGUGGAACUUUGUGGAAUCU |
| | | ATAGTTTGACAATAGTCAGT | | | CUCAGCAAGAAAUCCUAAAG |
| | | AATGCATGTGGAACTTTGTG | | | ACCAGGAAGCAUUAUGGGAC |
| | | GAATCTCTCAGCAAGAAAT | | | AUGGGGGCAGUUAGCAUGCU |
| | | CCTAAAGACCAGGAAGCAT | | | CAAGAACCUCAUUCAUUCAA |
| | | TATGGGACATGGGGGCAGT | | | AGCACAAAAUGAUUGCUAUG |
| | | TAGCATGCTCAAGAACCTCA | | | GGAAGUGCUGCAGCUUUAAG |
| | | TTCATTCAAAGCACAAAATG | | | GAAUCUCAUGGCAAAUAGGC |
| | | ATTGCTATGGGAAGTGCTGC | | | CUGCGAAGUACAAGGAUGCC |
| | | AGCTTTAAGGAATCTCATGG | | | AAUAUUAUGUCUCCUGGCUC |
| | | CAAATAGGCCTGCGAAGTA | | | AAGCUUGCCAUCUCUUCAUG |
| | | CAAGGATGCCAATATTATGT | | | UUAGGAAACAAAAAGCCCUA |
| | | CTCCTGGCTCAAGCTTGCCA | | | GAAGCAGAAUUAGAUGCUCA |
| | | TCTCTTCATGTTAGGAAACA | | | GCACUUAUCAGAAACUUUUG |
| | | AAAAGCCCTAGAAGCAGAA | | | ACAAUAUAGACAAUUUAAGU |
| | | TTAGATGCTCAGCACTTATC | | | CCCAAGGCAUCUCAUCGUAG |
| | | AGAAACTTTTGACAATATAG | | | UAAGCAGAGACACAAGCAAA |
| | | ACAATTTAAGTCCCAAGGC | | | GUCUCUAUGGUGAUUAUGUU |
| | | ATCTCATCGTAGTAAGCAGA | | | UUUGACACCAAUCGACAUGA |
| | | GACACAAGCAAAGTCTCTA | | | UGAUAAUAGGUCAGACAAUU |
| | | TGGTGATTATGTTTTTGACA | | | UUAAUACUGGCAACAUGACU |
| | | CCAATCGACATGATGATAAT | | | GUCCUUUCACCAUAUUUGAA |
| | | AGGTCAGACAATTTTAATAC | | | UACUACAGUGUUACCCAGCU |
| | | TGGCAACATGACTGTCCTTT | | | CCUCUUCAUCAAGAGGAAGC |
| | | CACCATATTTGAATACTACA | | | UUAGAUAGUUCUCGUUCUGA |
| | | GTGTTACCCAGCTCCTCTTC | | | AAAAGAUAGAAGUUUGGAGA |
| | | ATCAAGAGGAAGCTTAGAT | | | GAGAACGCGGAAUUGGUCUA |
| | | AGTTCTCGTTCTGAAAAAGA | | | GGCAACUACCAUCCAGCAAC |
| | | TAGAAGTTTGGAGAGAGAA | | | AGAAAAUCCAGGAACUUCUU |
| | | CGCGGAATTGGTCTAGGCA | | | CAAAGCGAGGUUUGCAGAUC |
| | | ACTACCATCCAGCAACAGA | | | UCCACCACUGCAGCCCAGAU |
| | | AAATCCAGGAACTTCTTCAA | | | UGCCAAAGUCAUGGAAGAAG |
| | | AGCGAGGTTTGCAGATCTCC | | | UGUCAGCCAUUCAUACCUCU |
| | | ACCACTGCAGCCCAGATTGC | | | CAGGAAGACAGAAGUUCUGG |
| | | CAAAGTCATGGAAGAAGTG | | | GUCUACCACUGAAUUACAUU |
| | | TCAGCCATTCATACCTCTCA | | | GUGUGACAGAUGAGAGAAAU |
| | | GGAAGACAGAAGTTCTGGG | | | GCACUUAGAAGAAGCUCUGC |
| | | TCTACCACTGAATTACATTG | | | UGCCCAUACACAUUCAAACA |
| | | TGTGACAGATGAGAGAAAT | | | CUUACAAUUUCACUAAGUCG |
| | | GCACTTAGAAGAAGCTCTG | | | GAAAAUUCAAAUAGGACAUG |
| | | CTGCCCATACACATTCAAAC | | | UUCUAUGCCUUAUGCCAAAU |
| | | ACTTACAATTTCACTAAGTC | | | UAGAAUACAAGAGAUCUUCA |
| | | GGAAAATTCAAATAGGACA | | | AAUGAUAGUUUAAAUAGUGU |
| | | TGTTCTATGCCTTATGCCAA | | | CAGUAGUAGUGAUGGUUAUG |
| | | ATTAGAATACAAGAGATCTT | | | GUAAAAGAGGUCAAAUGAAA |
| | | CAAATGATAGTTTAAATAGT | | | CCCUCGAUUGAAUCCUAUUC |
| | | GTCAGTAGTAGTGATGGTTA | | | UGAAGAUGAUGAAAGUAAGU |
| | | TGGTAAAAGAGGTCAAATG | | | UUUGCAGUUAUGGUCAAUAC |
| | | AAACCCTCGATTGAATCCTA | | | CCAGCCGACCUAGCCCAUAA |
| | | TTCTGAAGATGATGAAAGT | | | AAUACAUAGUGCAAAUCAUA |
| | | AAGTTTTGCAGTTATGGTCA | | | UGGAUGAUAAUGAUGGAGAA |
| | | ATACCCAGCCGACCTAGCCC | | | CUAGAUACACCAAUAAAUUA |
| | | ATAAAATACATAGTGCAAA | | | UAGUCUUAAAUAUUCAGAUG |
| | | TCATATGGATGATAATGATG | | | AGCAGUUGAACUCUGGAAGG |
| | | GAGAACTAGATACACCAAT | | | CAAAGUCCUUCACAGAAUGA |
| | | AAATTATAGTCTTAAATATT | | | AAGAUGGGCAAGACCCAAAC |
| | | CAGATGAGCAGTTGAACTCT | | | ACAUAAUAGAAGAUGAAAUA |
| | | GGAAGGCAAAGTCCTTCAC | | | AAACAAAGUGAGCAAAGACA |
| | | AGAATGAAAGATGGGCAAG | | | AUCAAGGAAUCAAAGUACAA |
| | | ACCCAAACACATAATAGAA | | | CUUAUCCUGUUUAUACUGAG |
| | | GATGAAATAAAACAAAGTG | | | AGCACUGAUGAUAAACACCU |
| | | AGCAAAGACAATCAAGGAA | | | CAAGUUCCAACCACAUUUUG |
| | | TCAAAGTACAACTTATCCTG | | | GACAGCAGGAAUGUGUUUCU |
| | | TTTATACTGAGAGCACTGAT | | | CCAUACAGGUCACGGGGAGC |
| | | GATAAACACCTCAAGTTCCA | | | CAAUGGUUCAGAAACAAAUC |
| | | ACCACATTTTGGACAGCAG | | | GAGUGGGUUCUAAUCAUGGA |
| | | GAATGTGTTTCTCCATACAG | | | AUUAAUCAAAAUGUAAGCCA |
| | | GTCACGGGGAGCCAATGGT | | | GUCUUUGUGUCAAGAAGAUG |
| | | TCAGAAACAAATCGAGTGG | | | ACUAUGAAGAUGAUAAGCCU |
| | | GTTCTAATCATGGAATTAAT | | | ACCAAUUAUAGUGAACGUUA |
| | | CAAAATGTAAGCCAGTCTTT | | | CUCUGAAGAAGAACAGCAUG |
| | | GTGTCAAGAAGATGACTAT | | | AAGAAGAAGAGAGACCAACA |
| | | GAAGATGATAAGCCTACCA | | | AAUUAUAGCAUAAAAUAUAA |
| | | ATTATAGTGAACGTTACTCT | | | UGAAGAGAAACGUCAUGUGG |
| | | GAAGAAGAACAGCATGAAG | | | AUCAGCCUAUUGAUUAUAGU |
| | | AAGAAGAGAGACCAACAAA | | | UUAAAAUAUGCCACAGAUAU |
| | | TTATAGCATAAAATATAATG | | | UCCUUCAUCACAGAAACAGU |
| | | AAGAGAAACGTCATGTGGA | | | CAUUUUCAUUCUCAAAGAGU |
| | | TCAGCCTATTGATTATAGTT | | | UCAUCUGGACAAAGCAGUAA |
| | | TAAAATATGCCACAGATATT | | | AACCGAACAUAUGUCUUCAA |
| | | CCTTCATCACAGAAACAGTC | | | GCAGUGAGAAUACGUCCACA |
| | | ATTTTCATTCTCAAAGAGTT | | | CCUUCAUCUAAUGCCAAGAG |
| | | CATCTGGACAAAGCAGTAA | | | GCAGAAUCAGCUCCAUCCAA |
| | | AACCGAACATATGTCTTCAA | | | GUUCUGCACAGAGUAGAAGU |
| | | GCAGTGAGAATACGTCCAC | | | GGUCAGCCUCAAAAGGCUGC |
| | | ACCTTCATCTAATGCCAAGA | | | CACUUGCAAAGUUUCUUCUA |
| | | GGCAGAATCAGCTCCATCC | | | UUAACCAAGAAACAAUACAG |
| | | AAGTTCTGCACAGAGTAGA | | | ACUUAUUGUGUAGAAGAUAC |
| | | AGTGGTCAGCCTCAAAAGG | | | UCCAAUAUGUUUUUCAAGAU |
| | | CTGCCACTTGCAAAGTTTCT | | | GUAGUUCAUUAUCAUCUUUG |
| | | TCTATTAACCAAGAAACAAT | | | UCAUCAGCUGAAGAUGAAAU |
| | | ACAGACTTATTGTGTAGAAG | | | AGGAUGUAAUCAGACGACAC |
| | | ATACTCCAATATGTTTTTCA | | | AGGAAGCAGAUUCUGCUAAU |
| | | AGATGTAGTTCATTATCATC | | | ACCCUGCAAAUAGCAGAAAU |
| | | TTTGTCATCAGCTGAAGATG | | | AAAAGAAAAGAUUGGAACUA |
| | | AAATAGGATGTAATCAGAC | | | GGUCAGCUGAAGAUCCUGUG |
| | | GACACAGGAAGCAGATTCT | | | AGCGAAGUUCCAGCAGUGUC |
| | | GCTAATACCCTGCAAATAGC | | | ACAGCACCCUAGAACCAAAU |
| | | AGAAATAAAAGAAAAGATT | | | CCAGCAGACUGCAGGGUUCU |
| | | GGAACTAGGTCAGCTGAAG | | | AGUUUAUCUUCAGAAUCAGC |
| | | ATCCTGTGAGCGAAGTTCCA | | | CAGGCACAAAGCUGUUGAAU |
| | | GCAGTGTCACAGCACCCTA | | | UUUCUUCAGGAGCGAAAUCU |
| | | GAACCAAATCCAGCAGACT | | | CCCUCCAAAAGUGGUGCUCA |
| | | GCAGGGTTCTAGTTTATCTT | | | GACACCCAAAAGUCCACCUG |
| | | CAGAATCAGCCAGGCACAA | | | AACACUAUGUUCAGGAGACC |
| | | AGCTGTTGAATTTTCTTCAG | | | CCACUCAUGUUUAGCAGAUG |
| | | GAGCGAAATCTCCCTCCAA | | | UACUUCUGUCAGUUCACUUG |
| | | AAGTGGTGCTCAGACACCC | | | AUAGUUUUGAGAGUCGUUCG |
| | | AAAAGTCCACCTGAACACT | | | AUUGCCAGCUCCGUUCAGAG |
| | | ATGTTCAGGAGACCCCACTC | | | UGAACCAUGCAGUGGAAUGG |
| | | ATGTTTAGCAGATGTACTTC | | | UAAGUGGCAUUAUAAGCCCC |
| | | TGTCAGTTCACTTGATAGTT | | | AGUGAUCUUCCAGAUAGCCC |
| | | TTGAGAGTCGTTCGATTGCC | | | UGGACAAACCAUGCCACCAA |
| | | AGCTCCGTTCAGAGTGAACC | | | GCAGAAGUAAAACACCUCCA |
| | | ATGCAGTGGAATGGTAAGT | | | CCACCUCCUCAAACAGCUCAA |
| | | GGCATTATAAGCCCCAGTG | | | ACCAAGCGAGAAGUACCUAA |
| | | ATCTTCCAGATAGCCCTGGA | | | AAAUAAAGCACCUACUGCUG |
| | | CAAACCATGCCACCAAGCA | | | AAAAGAGAGAGAGUGGACCU |
| | | GAAGTAAAACACCTCCACC | | | AAGCAAGCUGCAGUAAAUGC |
| | | ACCTCCTCAAACAGCTCAAA | | | UGCAGUUCAGAGGGUCCAGG |
| | | CCAAGCGAGAAGTACCTAA | | | UUCUUCCAGAUGCUGAUACU |
| | | AAATAAAGCACCTACTGCT | | | UUAUUACAUUUUGCCACGGA |
| | | GAAAAGAGAGAGAGTGGAC | | | AAGUACUCCAGAUGGAUUUU |
| | | CTAAGCAAGCTGCAGTAAA | | | CUUGUUCAUCCAGCCUGAGU |
| | | TGCTGCAGTTCAGAGGGTCC | | | GCUCUGAGCCUCGAUGAGCC |
| | | AGGTTCTTCCAGATGCTGAT | | | AUUUAUACAGAAAGAUGUGG |
| | | ACTTTATTACATTTTGCCAC | | | AAUUAAGAAUAAUGCCUCCA |
| | | GGAAAGTACTCCAGATGGA | | | GUUCAGGAAAAUGACAAUGG |
| | | TTTTCTTGTTCATCCAGCCT | | | GAAUGAAACAGAAUCAGAGC |
| | | GAGTGCTCTGAGCCTCGATG | | | AGCCUAAAGAAUCAAAUGAA |
| | | AGCCATTTATACAGAAAGA | | | AACCAAGAGAAAGAGGCAGA |
| | | TGTGGAATTAAGAATAATG | | | AAAAACUAUUGAUUCUGAAA |
| | | CCTCCAGTTCAGGAAAATG | | | AGGACCUAUUAGAUGAUUCA |
| | | ACAATGGGAATGAAACAGA | | | GAUGAUGAUGAUAUUGAAAU |
| | | ATCAGAGCAGCCTAAAGAA | | | ACUAGAAGAAUGUAUUAUUU |
| | | TCAAATGAAAACCAAGAGA | | | CUGCCAUGCCAACAAAGUCA |
| | | AAGAGGCAGAAAAAACTAT | | | UCACGUAAAGCAAAAAAGCC |
| | | TGATTCTGAAAAGGACCTAT | | | AGCCCAGACUGCUUCAAAAU |
| | | TAGATGATTCAGATGATGAT | | | UACCUCCACCUGUGGCAAGG |
| | | GATATTGAAATACTAGAAG | | | AAACCAAGUCAGCUGCCUGU |
| | | AATGTATTATTTCTGCCATG | | | GUACAAACUUCUACCAUCAC |
| | | CCAACAAAGTCATCACGTA | | | AAAACAGGUUGCAACCCCAA |
| | | AAGCAAAAAAGCCAGCCCA | | | AAGCAUGUUAGUUUUACACC |
| | | GACTGCTTCAAAATTACCTC | | | GGGGGAUGAUAUGCCACGGG |
| | | CACCTGTGGCAAGGAAACC | | | UGUAUUGUGUUGAAGGGACA |
| | | AAGTCAGCTGCCTGTGTACA | | | CCUAUAAACUUUUCCACAGC |
| | | AACTTCTACCATCACAAAAC | | | UACAUCUCUAAGUGAUCUAA |
| | | AGGTTGCAACCCCAAAAGC | | | CAAUCGAAUCCCCUCCAAAU |
| | | ATGTTAGTTTTACACCGGGG | | | GAGUUAGCUGCUGGAGAAGG |
| | | GATGATATGCCACGGGTGT | | | AGUUAGAGGAGGGGCACAGU |
| | | ATTGTGTTGAAGGGACACCT | | | CAGGUGAAUUUGAAAAACGA |
| | | ATAAACTTTTCCACAGCTAC | | | GAUACCAUUCCUACAGAAGG |
| | | ATCTCTAAGTGATCTAACAA | | | CAGAAGUACAGAUGAGGCUC |
| | | TCGAATCCCCTCCAAATGAG | | | AAGGAGGAAAAACCUCAUCU |
| | | TTAGCTGCTGGAGAAGGAG | | | GUAACCAUACCUGAAUUGGA |
| | | TTAGAGGAGGGGCACAGTC | | | UGACAAUAAAGCAGAGGAAG |
| | | AGGTGAATTTGAAAAACGA | | | GUGAUAUUCUUGCAGAAUGC |
| | | GATACCATTCCTACAGAAG | | | AUUAAUUCUGCUAUGCCCAA |
| | | GCAGAAGTACAGATGAGGC | | | AGGGAAAAGUCACAAGCCUU |
| | | TCAAGGAGGAAAAACCTCA | | | UCCGUGUGAAAAAGAUAAUG |
| | | TCTGTAACCATACCTGAATT | | | GACCAGGUCCAGCAAGCAUC |
| | | GGATGACAATAAAGCAGAG | | | UGCGUCUUCUUCUGCACCCA |
| | | GAAGGTGATATTCTTGCAGA | | | ACAAAAAUCAGUUAGAUGGU |
| | | ATGCATTAATTCTGCTATGC | | | AAGAAAAAGAAACCAACUUC |
| | | CCAAAGGGAAAAGTCACAA | | | ACCAGUAAAACCUAUACCAC |
| | | GCCTTTCCGTGTGAAAAAGA | | | AAAAUACUGAAUAUAGGACA |
| | | TAATGGACCAGGTCCAGCA | | | CGUGUAAGAAAAAAUGCAGA |
| | | AGCATCTGCGTCTTCTTCTG | | | CUCAAAAAAUAAUUUAAAUG |
| | | CACCCAACAAAAATCAGTT | | | CUGAGAGAGUUUUCUCAGAC |
| | | AGATGGTAAGAAAAAGAAA | | | AACAAAGAUUCAAAGAAACA |
| | | CCAACTTCACCAGTAAAACC | | | GAAUUUGAAAAAUAAUUCCA |
| | | TATACCACAAAATACTGAAT | | | AGGUCUUCAAUGAUAAGCUC |
| | | ATAGGACACGTGTAAGAAA | | | CCAAAUAAUGAAGAUAGAGU |
| | | AAATGCAGACTCAAAAAAT | | | CAGAGGAAGUUUUGCUUUUG |
| | | AATTTAAATGCTGAGAGAG | | | AUUCACCUCAUCAUUACACG |
| | | TTTTCTCAGACAACAAAGAT | | | CCUAUUGAAGGAACUCCUUA |
| | | TCAAAGAAACAGAATTTGA | | | CUGUUUUUCACGAAAUGAUU |
| | | AAAATAATTCCAAGGTCTTC | | | CUUUGAGUUCUCUAGAUUUU |
| | | AATGATAAGCTCCCAAATA | | | GAUGAUGAUGAUGUUGACCU |
| | | ATGAAGATAGAGTCAGAGG | | | UUCCAGGGAAAAGGCUGAAU |
| | | AAGTTTTGCTTTTGATTCAC | | | UAAGAAAGGCAAAAGAAAAU |
| | | CTCATCATTACACGCCTATT | | | AAGGAAUCAGAGGCUAAAGU |
| | | GAAGGAACTCCTTACTGTTT | | | UACCAGCCACACAGAACUAA |
| | | TTCACGAAATGATTCTTTGA | | | CCUCCAACCAACAAUCAGCU |
| | | GTTCTCTAGATTTTGATGAT | | | AAUAAGACACAAGCUAUUGC |
| | | GATGATGTTGACCTTTCCAG | | | AAAGCAGCCAAUAAAUCGAG |
| | | GGAAAAGGCTGAATTAAGA | | | GUCAGCCUAAACCCAUACUU |
| | | AAGGCAAAAGAAAATAAGG | | | CAGAAACAAUCCACUUUUCC |
| | | AATCAGAGGCTAAAGTTAC | | | CCAGUCAUCCAAAGACAUAC |
| | | CAGCCACACAGAACTAACC | | | CAGACAGAGGGGCAGCAACU |
| | | TCCAACCAACAATCAGCTA | | | GAUGAAAAGUUACAGAAUUU |
| | | ATAAGACACAAGCTATTGC | | | UGCUAUUGAAAAUACUCCGG |
| | | AAAGCAGCCAATAAATCGA | | | UUUGCUUUUCUCAUAAUUCC |
| | | GGTCAGCCTAAACCCATACT | | | UCUCUGAGUUCUCUCAGUGA |
| | | TCAGAAACAATCCACTTTTC | | | CAUUGACCAAGAAAACAACA |
| | | CCCAGTCATCCAAAGACAT | | | AUAAAGAAAAUGAACCUAUC |
| | | ACCAGACAGAGGGGCAGCA | | | AAAGAGACUGAGCCCCCUGA |
| | | ACTGATGAAAAGTTACAGA | | | CUCACAGGGAGAACCAAGUA |
| | | ATTTTGCTATTGAAAATACT | | | AACCUCAAGCAUCAGGCUAU |
| | | CCGGTTTGCTTTTCTCATAA | | | GCUCCUAAAUCAUUUCAUGU |
| | | TTCCTCTCTGAGTTCTCTCA | | | UGAAGAUACCCCAGUUUGUU |
| | | GTGACATTGACCAAGAAAA | | | UCUCAAGAAACAGUUCUCUC |
| | | CAACAATAAAGAAAATGAA | | | AGUUCUCUUAGUAUUGACUC |
| | | CCTATCAAAGAGACTGAGC | | | UGAAGAUGACCUGUUGCAGG |
| | | CCCCTGACTCACAGGGAGA | | | AAUGUAUAAGCUCCGCAAUG |
| | | ACCAAGTAAACCTCAAGCA | | | CCAAAAAAGAAAAAGCCUUC |
| | | TCAGGCTATGCTCCTAAATC | | | AAGACUCAAGGGUGAUAAUG |
| | | ATTTCATGTTGAAGATACCC | | | AAAAACAUAGUCCCAGAAAU |
| | | CAGTTTGTTTCTCAAGAAAC | | | AUGGGUGGCAUAUUAGGUGA |
| | | AGTTCTCTCAGTTCTCTTAG | | | AGAUCUGACACUUGAUUUGA |
| | | TATTGACTCTGAAGATGACC | | | AAGAUAUACAGAGACCAGAU |
| | | TGTTGCAGGAATGTATAAGC | | | UCAGAACAUGGUCUAUCCCC |
| | | TCCGCAATGCCAAAAAAGA | | | UGAUUCAGAAAAUUUUGAUU |
| | | AAAAGCCTTCAAGACTCAA | | | GGAAAGCUAUUCAGGAAGGU |
| | | GGGTGATAATGAAAAACAT | | | GCAAAUUCCAUAGUAAGUAG |
| | | AGTCCCAGAAATATGGGTG | | | UUUACAUCAAGCUGCUGCUG |
| | | GCATATTAGGTGAAGATCTG | | | CUGCAUGUUUAUCUAGACAA |
| | | ACACTTGATTTGAAAGATAT | | | GCUUCGUCUGAUUCAGAUUC |
| | | ACAGAGACCAGATTCAGAA | | | CAUCCUUUCCCUGAAAUCAG |
| | | CATGGTCTATCCCCTGATTC | | | GAAUCUCUCUGGGAUCACCA |
| | | AGAAAATTTTGATTGGAAA | | | UUUCAUCUUACACCUGAUCA |
| | | GCTATTCAGGAAGGTGCAA | | | AGAAGAAAAACCCUUUACAA |
| | | ATTCCATAGTAAGTAGTTTA | | | GUAAUAAAGGCCCACGAAUU |
| | | CATCAAGCTGCTGCTGCTGC | | | CUAAAACCAGGGGAGAAAAG |
| | | ATGTTTATCTAGACAAGCTT | | | UACAUUGGAAACUAAAAAGA |
| | | CGTCTGATTCAGATTCCATC | | | UAGAAUCUGAAAGUAAAGGA |
| | | CTTTCCCTGAAATCAGGAAT | | | AUCAAAGGAGGAAAAAAAGU |
| | | CTCTCTGGGATCACCATTTC | | | UUAUAAAAGUUUGAUUACUG |
| | | ATCTTACACCTGATCAAGAA | | | GAAAAGUUCGAUCUAAUUCA |
| | | GAAAAACCCTTTACAAGTA | | | GAAAUUUCAGGCCAAAUGAA |
| | | ATAAAGGCCCACGAATTCT | | | ACAGCCCCUUCAAGCAAACA |
| | | AAAACCAGGGGAGAAAAGT | | | UGCCUUCAAUCUCUCGAGGC |
| | | ACATTGGAAACTAAAAAGA | | | AGGACAAUGAUUCAUAUUCC |
| | | TAGAATCTGAAAGTAAAGG | | | AGGAGUUCGAAAUAGCUCCU |
| | | AATCAAAGGAGGAAAAAAA | | | CAAGUACAAGUCCUGUUUCU |
| | | GTTTATAAAAGTTTGATTAC | | | AAAAAAGGCCCACCCCUUAA |
| | | TGGAAAAGTTCGATCTAATT | | | GACUCCAGCCUCCAAAAGCCC |
| | | CAGAAATTTCAGGCCAAAT | | | UAGUGAAGGUCAAACAGCCA |
| | | GAAACAGCCCCTTCAAGCA | | | CCACUUCUCCUAGAGGAGCC |
| | | AACATGCCTTCAATCTCTCG | | | AAGCCAUCUGUGAAAUCAGA |
| | | AGGCAGGACAATGATTCAT | | | AUUAAGCCCUGUUGCCAGGC |
| | | ATTCCAGGAGTTCGAAATA | | | AGACAUCCCAAAUAGGUGGG |
| | | GCTCCTCAAGTACAAGTCCT | | | UCAAGUAAAGCACCUUCUAG |
| | | GTTTCTAAAAAAGGCCCACC | | | AUCAGGAUCUAGAGAUUCGA |
| | | CCTTAAGACTCCAGCCTCCA | | | CCCCUUCAAGACCUGCCCAGC |
| | | AAAGCCCTAGTGAAGGTCA | | | AACCAUUAAGUAGACCUAUA |
| | | AACAGCCACCACTTCTCCTA | | | CAGUCUCCUGGCCGAAACUC |
| | | GAGGAGCCAAGCCATCTGT | | | AAUUUCCCCUGGUAGAAAUG |
| | | GAAATCAGAATTAAGCCCT | | | GAAUAAGUCCUCCUAACAAA |
| | | GTTGCCAGGCAGACATCCC | | | UUAUCUCAACUUCCAAGGAC |
| | | AAATAGGTGGGTCAAGTAA | | | AUCAUCCCCUAGUACUGCUU |
| | | AGCACCTTCTAGATCAGGAT | | | CAACUAAGUCCUCAGGUUCU |
| | | CTAGAGATTCGACCCCTTCA | | | GGAAAAAUGUCAUAUACAUC |
| | | AGACCTGCCCAGCAACCATT | | | UCCAGGUAGACAGAUGAGCC |
| | | AAGTAGACCTATACAGTCTC | | | AACAGAACCUUACCAAACAA |
| | | CTGGCCGAAACTCAATTTCC | | | ACAGGUUUAUCCAAGAAUGC |
| | | CCTGGTAGAAATGGAATAA | | | CAGUAGUAUUCCAAGAAGUG |
| | | GTCCTCCTAACAAATTATCT | | | AGUCUGCCUCCAAAGGACUA |
| | | CAACTTCCAAGGACATCATC | | | AAUCAGAUGAAUAAUGGUAA |
| | | CCCTAGTACTGCTTCAACTA | | | UGGAGCCAAUAAAAAGGUAG |
| | | AGTCCTCAGGTTCTGGAAAA | | | AACUUUCUAGAAUGUCUUCA |
| | | ATGTCATATACATCTCCAGG | | | ACUAAAUCAAGUGGAAGUGA |
| | | TAGACAGATGAGCCAACAG | | | AUCUGAUAGAUCAGAAAGAC |
| | | AACCTTACCAAACAAACAG | | | CUGUAUUAGUACGCCAGUCA |
| | | GTTTATCCAAGAATGCCAGT | | | ACUUUCAUCAAAGAAGCUCC |
| | | AGTATTCCAAGAAGTGAGT | | | AAGCCCAACCUUAAGAAGAA |
| | | CTGCCTCCAAAGGACTAAAT | | | AAUUGGAGGAAUCUGCUUCA |
| | | CAGATGAATAATGGTAATG | | | UUUGAAUCUCUUUCUCCAUC |
| | | GAGCCAATAAAAAGGTAGA | | | AUCUAGACCAGCUUCUCCCA |
| | | ACTTTCTAGAATGTCTTCAA | | | CUAGGUCCCAGGCACAAACU |
| | | CTAAATCAAGTGGAAGTGA | | | CCAGUUUUAAGUCCUUCCCU |
| | | ATCTGATAGATCAGAAAGA | | | UCCUGAUAUGUCUCUAUCCA |
| | | CCTGTATTAGTACGCCAGTC | | | CACAUUCGUCUGUUCAGGCU |
| | | AACTTTCATCAAAGAAGCTC | | | GGUGGAUGGCGAAAACUCCC |
| | | CAAGCCCAACCTTAAGAAG | | | ACCUAAUCUCAGUCCCACUA |
| | | AAAATTGGAGGAATCTGCTT | | | UAGAGUAUAAUGAUGGAAGA |
| | | CATTTGAATCTCTTTCTCCA | | | CCAGCAAAGCGCCAUGAUAU |
| | | TCATCTAGACCAGCTTCTCC | | | UGCACGGUCUCAUUCUGAAA |
| | | CACTAGGTCCCAGGCACAA | | | GUCCUUCUAGACUUCCAAUC |
| | | ACTCCAGTTTTAAGTCCTTC | | | AAUAGGUCAGGAACCUGGAA |
| | | CCTTCCTGATATGTCTCTAT | | | ACGUGAGCACAGCAAACAUU |
| | | CCACACATTCGTCTGTTCAG | | | CAUCAUCCCUUCCUCGAGUA |
| | | GCTGGTGGATGGCGAAAAC | | | AGCACUUGGAGAAGAACUGG |
| | | TCCCACCTAATCTCAGTCCC | | | AAGUUCAUCUUCAAUUCUUU |
| | | ACTATAGAGTATAATGATG | | | CUGCUUCAUCAGAAUCCAGU |
| | | GAAGACCAGCAAAGCGCCA | | | GAAAAAGCAAAAAGUGAGGA |
| | | TGATATTGCACGGTCTCATT | | | UGAAAAACAUGUGAACUCUA |
| | | CTGAAAGTCCTTCTAGACTT | | | UUUCAGGAACCAAACAAAGU |
| | | CCAATCAATAGGTCAGGAA | | | AAAGAAAACCAAGUAUCCGC |
| | | CCTGGAAACGTGAGCACAG | | | AAAAGGAACAUGGAGAAAAA |
| | | CAAACATTCATCATCCCTTC | | | UAAAAGAAAAUGAAUUUUCU |
| | | CTCGAGTAAGCACTTGGAG | | | CCCACAAAUAGUACUUCUCA |
| | | AAGAACTGGAAGTTCATCTT | | | GACCGUUUCCUCAGGUGCUA |
| | | CAATTCTTTCTGCTTCATCA | | | CAAAUGGUGCUGAAUCAAAG |
| | | GAATCCAGTGAAAAAGCAA | | | ACUCUAAUUUAUCAAAUGGC |
| | | AAAGTGAGGATGAAAAACA | | | ACCUGCUGUUUCUAAAACAG |
| | | TGTGAACTCTATTTCAGGAA | | | AGGAUGUUUGGGUGAGAAUU |
| | | CCAAACAAAGTAAAGAAAA | | | GAGGACUGUCCCAUUAACAA |
| | | CCAAGTATCCGCAAAAGGA | | | UCCUAGAUCUGGAAGAUCUC |
| | | ACATGGAGAAAAATAAAAG | | | CCACAGGUAAUACUCCCCCG |
| | | AAAATGAATTTTCTCCCACA | | | GUGAUUGACAGUGUUUCAGA |
| | | AATAGTACTTCTCAGACCGT | | | AAAGGCAAAUCCAAACAUUA |
| | | TTCCTCAGGTGCTACAAATG | | | AAGAUUCAAAAGAUAAUCAG |
| | | GTGCTGAATCAAAGACTCTA | | | GCAAAACAAAAUGUGGGUAA |
| | | ATTTATCAAATGGCACCTGC | | | UGGCAGUGUUCCCAUGCGUA |
| | | TGTTTCTAAAACAGAGGATG | | | CCGUGGGUUUGGAAAAUCGC |
| | | TTTGGGTGAGAATTGAGGA | | | CUGAACUCCUUUAUUCAGGU |
| | | CTGTCCCATTAACAATCCTA | | | GGAUGCCCCUGACCAAAAAG |
| | | GATCTGGAAGATCTCCCACA | | | GAACUGAGAUAAAACCAGGA |
| | | GGTAATACTCCCCCGGTGAT | | | CAAAAUAAUCCUGUCCCUGU |
| | | TGACAGTGTTTCAGAAAAG | | | AUCAGAGACUAAUGAAAGUU |
| | | GCAAATCCAAACATTAAAG | | | CUAUAGUGGAACGUACCCCA |
| | | ATTCAAAAGATAATCAGGC | | | UUCAGUUCUAGCAGCUCAAG |
| | | AAAACAAAATGTGGGTAAT | | | CAAACACAGUUCACCUAGUG |
| | | GGCAGTGTTCCCATGCGTAC | | | GGACUGUUGCUGCCAGAGUG |
| | | CGTGGGTTTGGAAAATCGCC | | | ACUCCUUUUAAUUACAACCC |
| | | TGAACTCCTTTATTCAGGTG | | | AAGCCCUAGGAAAAGCAGCG |
| | | GATGCCCCTGACCAAAAAG | | | CAGAUAGCACUUCAGCUCGG |
| | | GAACTGAGATAAAACCAGG | | | CCAUCUCAGAUCCCAACUCCA |
| | | ACAAAATAATCCTGTCCCTG | | | GUGAAUAACAACACAAAGAA |
| | | TATCAGAGACTAATGAAAG | | | GCGAGAUUCCAAAACUGACA |
| | | TTCTATAGTGGAACGTACCC | | | GCACAGAAUCCAGUGGAACC |
| | | CATTCAGTTCTAGCAGCTCA | | | CAAAGUCCUAAGCGCCAUUC |
| | | AGCAAACACAGTTCACCTA | | | UGGGUCUUACCUUGUGACAU |
| | | GTGGGACTGTTGCTGCCAGA | | | CUGUUUAA |
| | | GTGACTCCTTTTAATTACAA | | | |
| | | CCCAAGCCCTAGGAAAAGC | | | |
| | | AGCGCAGATAGCACTTCAG | | | |
| | | CTCGGCCATCTCAGATCCCA | | | |
| | | ACTCCAGTGAATAACAACA | | | |
| | | CAAAGAAGCGAGATTCCAA | | | |
| | | AACTGACAGCACAGAATCC | | | |
| | | AGTGGAACCCAAAGTCCTA | | | |
| | | AGCGCCATTCTGGGTCTTAC | | | |
| | | CTTGTGACATCTGTTTAA | | | |
| 9 | WT | ATGGCGAGTAGCAGCGGCT | 10 | WT | AUGGCGAGUAGCAGCGGCUC |
| | CSNK1 | CCAAGGCTGAATTCATTGTC | | CSNK1 | CAAGGCUGAAUUCAUUGUCG |
| | A1 | GGAGGGAAATATAAACTGG | | A1 | GAGGGAAAUAUAAACUGGUA |
| | DNA | TACGGAAGATCGGGTCTGG | | RNA | CGGAAGAUCGGGUCUGGCUC |
| | | CTCCTTCGGGGACATCTATT | | | CUUCGGGGACAUCUAUUUGG |
| | | TGGCGATCAACATCACCAA | | | CGAUCAACAUCACCAACGGC |
| | | CGGCGAGGAAGTGGCAGTG | | | GAGGAAGUGGCAGUGAAGCU |
| | | AAGCTAGAATCTCAGAAGG | | | AGAAUCUCAGAAGGCCAGGC |
| | | CCAGGCATCCCCAGTTGCTG | | | AUCCCCAGUUGCUGUACGAG |
| | | TACGAGAGCAAGCTCTATA | | | AGCAAGCUCUAUAAGAUUCU |
| | | AGATTCTTCAAGGTGGGGTT | | | UCAAGGUGGGGUUGGCAUCC |
| | | GGCATCCCCCACATACGGTG | | | CCCACAUACGGUGGUAUGGU |
| | | GTATGGTCAGGAAAAAGAC | | | CAGGAAAAAGACUACAAUGU |
| | | TACAATGTACTAGTCATGGA | | | ACUAGUCAUGGAUCUUCUGG |
| | | TCTTCTGGGACCTAGCCTCG | | | GACCUAGCCUCGAAGACCUC |
| | | AAGACCTCTTCAATTTCTGT | | | UUCAAUUUCUGUUCAAGAAG |
| | | TCAAGAAGGTTCACAATGA | | | GUUCACAAUGAAAACUGUAC |
| | | AAACTGTACTTATGTTAGCT | | | UUAUGUUAGCUGACCAGAUG |
| | | GACCAGATGATCAGTAGAA | | | AUCAGUAGAAUUGAAUAUGU |
| | | TTGAATATGTGCATACAAAG | | | GCAUACAAAGAAUUUUAUAC |
| | | AATTTTATACACAGAGACAT | | | ACAGAGACAUUAAACCAGAU |
| | | TAAACCAGATAACTTCCTAA | | | AACUUCCUAAUGGGUAUUGG |
| | | TGGGTATTGGGCGTCACTGT | | | GCGUCACUGUAAUAAGUUAU |
| | | AATAAGTTATTCCTTATTGA | | | UCCUUAUUGAUUUUGGUUUG |
| | | TTTTGGTTTGGCCAAAAAGT | | | GCCAAAAAGUACAGAGACAA |
| | | ACAGAGACAACAGGACAAG | | | CAGGACAAGGCAACACAUAC |
| | | GCAACACATACCATACAGA | | | CAUACAGAGAAGAUAAAAAC |
| | | GAAGATAAAAACCTCACTG | | | CUCACUGGCACUGCCCGAUA |
| | | GCACTGCCCGATATGCTAGC | | | UGCUAGCAUCAAUGCACAUC |
| | | ATCAATGCACATCTTGGTAT | | | UUGGUAUUGAGCAGAGUCGC |
| | | TGAGCAGAGTCGCCGAGAT | | | CGAGAUGACAUGGAAUCAUU |
| | | GACATGGAATCATTAGGAT | | | AGGAUAUGUUUUGAUGUAUU |
| | | ATGTTTTGATGTATTTTAAT | | | UUAAUAGAACCAGCCUGCCA |
| | | AGAACCAGCCTGCCATGGC | | | UGGCAAGGGCUAAAGGCUGC |
| | | AAGGGCTAAAGGCTGCAAC | | | AACAAAGAAACAAAAAUAUG |
| | | AAAGAAACAAAAATATGAA | | | AAAAGAUUAGUGAAAAGAAG |
| | | AAGATTAGTGAAAAGAAGA | | | AUGUCCACGCCUGUUGAAGU |
| | | TGTCCACGCCTGTTGAAGTT | | | UUUAUGUAAGGGGUUUCCUG |
| | | TTATGTAAGGGGTTTCCTGC | | | CAGAAUUUGCGAUGUACUUA |
| | | AGAATTTGCGATGTACTTAA | | | AACUAUUGUCGUGGGCUACG |
| | | ACTATTGTCGTGGGCTACGC | | | CUUUGAGGAAGCCCCAGAUU |
| | | TTTGAGGAAGCCCCAGATTA | | | ACAUGUAUCUGAGGCAGCUA |
| | | CATGTATCTGAGGCAGCTAT | | | UUCCGCAUUCUUUUCAGGAC |
| | | TCCGCATTCTTTTCAGGACC | | | CCUGAACCAUCAAUAUGACU |
| | | CTGAACCATCAATATGACTA | | | ACACAUUUGAUUGGACAAUG |
| | | CACATTTGATTGGACAATGT | | | UUAAAGCAGAAAGCAGCACA |
| | | TAAAGCAGAAAGCAGCACA | | | GCAGGCAGCCUCUUCCAGUG |
| | | GCAGGCAGCCTCTTCCAGTG | | | GGCAGGGUCAGCAGGCCCAA |
| | | GGCAGGGTCAGCAGGCCCA | | | ACCCCCACAGGCAAGCAAAC |
| | | AACCCCCACAGGCAAGCAA | | | UGACAAAACCAAGAGUAACA |
| | | ACTGACAAAACCAAGAGTA | | | UGAAAGGUUUCUAA |
| | | ACATGAAAGGTTTCTAA | | | |
| 11 | WT | ATGTCAGGGCGGCCCAGAA | 12 | WT | AUGUCAGGGCGGCCCAGAAC |
| | GSK3B | CCACCTCCTTTGCGGAGAGC | | GSK3B | CACCUCCUUUGCGGAGAGCU |
| | DNA | TGCAAGCCGGTGCAGCAGC | | RNA | GCAAGCCGGUGCAGCAGCCU |
| | | CTTCAGCTTTTGGCAGCATG | | | UCAGCUUUUGGCAGCAUGAA |
| | | AAAGTTAGCAGAGACAAGG | | | AGUUAGCAGAGACAAGGACG |
| | | ACGGCAGCAAGGTGACAAC | | | GCAGCAAGGUGACAACAGUG |
| | | AGTGGTGGCAACTCCTGGG | | | GUGGCAACUCCUGGGCAGGG |
| | | CAGGGTCCAGACAGGCCAC | | | UCCAGACAGGCCACAAGAAG |
| | | AAGAAGTCAGCTATACAGA | | | UCAGCUAUACAGACACUAAA |
| | | CACTAAAGTGATTGGAAAT | | | GUGAUUGGAAAUGGAUCAUU |
| | | GGATCATTTGGTGTGGTATA | | | UGGUGUGGUAUAUCAAGCCA |
| | | TCAAGCCAAACTTTGTGATT | | | AACUUUGUGAUUCAGGAGAA |
| | | CAGGAGAACTGGTCGCCAT | | | CUGGUCGCCAUCAAGAAAGU |
| | | CAAGAAAGTATTGCAGGAC | | | AUUGCAGGACAAGAGAUUUA |
| | | AAGAGATTTAAGAATCGAG | | | AGAAUCGAGAGCUCCAGAUC |
| | | AGCTCCAGATCATGAGAAA | | | AUGAGAAAGCUAGAUCACUG |
| | | GCTAGATCACTGTAACATAG | | | UAACAUAGUCCGAUUGCGUU |
| | | TCCGATTGCGTTATTTCTTCT | | | AUUUCUUCUACUCCAGUGGU |
| | | ACTCCAGTGGTGAGAAGAA | | | GAGAAGAAAGAUGAGGUCUA |
| | | AGATGAGGTCTATCTTAATC | | | UCUUAAUCUGGUGCUGGACU |
| | | TGGTGCTGGACTATGTTCCG | | | AUGUUCCGGAAACAGUAUAC |
| | | GAAACAGTATACAGAGTTG | | | AGAGUUGCCAGACACUAUAG |
| | | CCAGACACTATAGTCGAGC | | | UCGAGCCAAACAGACGCUCC |
| | | CAAACAGACGCTCCCTGTG | | | CUGUGAUUUAUGUCAAGUUG |
| | | ATTTATGTCAAGTTGTATAT | | | UAUAUGUAUCAGCUGUUCCG |
| | | GTATCAGCTGTTCCGAAGTT | | | AAGUUUAGCCUAUAUCCAUU |
| | | TAGCCTATATCCATTCCTTT | | | CCUUUGGAAUCUGCCAUCGG |
| | | GGAATCTGCCATCGGGATAT | | | GAUAUUAAACCGCAGAACCU |
| | | TAAACCGCAGAACCTCTTGT | | | CUUGUUGGAUCCUGAUACUG |
| | | TGGATCCTGATACTGCTGTA | | | CUGUAUUAAAACUCUGUGAC |
| | | TTAAAACTCTGTGACTTTGG | | | UUUGGAAGUGCAAAGCAGCU |
| | | AAGTGCAAAGCAGCTGGTC | | | GGUCCGAGGAGAACCCAAUG |
| | | CGAGGAGAACCCAATGTTT | | | UUUCGUAUAUCUGUUCUCGG |
| | | CGTATATCTGTTCTCGGTAC | | | UACUAUAGGGCACCAGAGUU |
| | | TATAGGGCACCAGAGTTGA | | | GAUCUUUGGAGCCACUGAUU |
| | | TCTTTGGAGCCACTGATTAT | | | AUACCUCUAGUAUAGAUGUA |
| | | ACCTCTAGTATAGATGTATG | | | UGGUCUGCUGGCUGUGUGUU |
| | | GTCTGCTGGCTGTGTGTTGG | | | GGCUGAGCUGUUACUAGGAC |
| | | CTGAGCTGTTACTAGGACAA | | | AACCAAUAUUUCCAGGGGAU |
| | | CCAATATTTCCAGGGGATAG | | | AGUGGUGUGGAUCAGUUGGU |
| | | TGGTGTGGATCAGTTGGTAG | | | AGAAAUAAUCAAGGUCCUGG |
| | | AAATAATCAAGGTCCTGGG | | | GAACUCCAACAAGGGAGCAA |
| | | AACTCCAACAAGGGAGCAA | | | AUCAGAGAAAUGAACCCAAA |
| | | ATCAGAGAAATGAACCCAA | | | CUACACAGAAUUUAAAUUCC |
| | | ACTACACAGAATTTAAATTC | | | CUCAAAUUAAGGCACAUCCU |
| | | CCTCAAATTAAGGCACATCC | | | UGGACUAAGGAUUCGUCAGG |
| | | TTGGACTAAGGATTCGTCAG | | | AACAGGACAUUUCACCUCAG |
| | | GAACAGGACATTTCACCTCA | | | GAGUGCGGGUCUUCCGACCC |
| | | GGAGTGCGGGTCTTCCGACC | | | CGAACUCCACCGGAGGCAAU |
| | | CCGAACTCCACCGGAGGCA | | | UGCACUGUGUAGCCGUCUGC |
| | | ATTGCACTGTGTAGCCGTCT | | | UGGAGUAUACACCAACUGCC |
| | | GCTGGAGTATACACCAACT | | | CGACUAACACCACUGGAAGC |
| | | GCCCGACTAACACCACTGG | | | UUGUGCACAUUCAUUUUUUG |
| | | AAGCTTGTGCACATTCATTT | | | AUGAAUUACGGGACCCAAAU |
| | | TTTGATGAATTACGGGACCC | | | GUCAAACUACCAAAUGGGCG |
| | | AAATGTCAAACTACCAAAT | | | AGACACACCUGCACUCUUCA |
| | | GGGCGAGACACACCTGCAC | | | ACUUCACCACUCAAGAACUG |
| | | TCTTCAACTTCACCACTCAA | | | UCAAGUAAUCCACCUCUGGC |
| | | GAACTGTCAAGTAATCCACC | | | UACCAUCCUUAUUCCUCCUC |
| | | TCTGGCTACCATCCTTATTC | | | AUGCUCGGAUUCAAGCAGCU |
| | | CTCCTCATGCTCGGATTCAA | | | GCUUCAACCCCCACAAAUGCC |
| | | GCAGCTGCTTCAACCCCCAC | | | ACAGCAGCGUCAGAUGCUAA |
| | | AAATGCCACAGCAGCGTCA | | | UACUGGAGACCGUGGACAGA |
| | | GATGCTAATACTGGAGACC | | | CCAAUAAUGCUGCUUCUGCA |
| | | GTGGACAGACCAATAATGC | | | UCAGCUUCCAACUCCACCUG |
| | | TGCTTCTGCATCAGCTTCCA | | | A |
| | | ACTCCACCTGA | | | |

TABLE 3
WT Protein Sequences

| SEQ ID | | |
|---|---|---|
| NO | Name | Protein Sequence |
| 13 | WT | MATQADLMELDMAMEPDRKAAVSHWQQQSYLDSGIHSGATTTAPSLSGKGNPEEED |
| | CTNNB1 | VDTSQVLYEWEQGFSQSFTQEQVADIDGQYAMTRAQRVRAAMFPETLDEGMQIPSTQ |
| | | FDAAHPTNVQRLAEPSQMLKHAVVNLINYQDDAELATRAIPELTKLLNDEDQVVVNK |
| | | AAVMVHQLSKKEASRHAIMRSPQMVSAIVRTMQNTNDVETARCTAGTLHNLSHHRE |
| | | GLLAIFKSGGIPALVKMLGSPVDSVLFYAITTLHNLLLHQEGAKMAVRLAGGLQKMV |
| | | ALLNKTNVKFLAITTDCLQILAYGNQESKLIILASGGPQALVNIMRTYTYEKLLWTTSR |
| | | VLKVLSVCSSNKPAIVEAGGMQALGLHLTDPSQRLVQNCLWTLRNLSDAATKQEGM |
| | | EGLLGTLVQLLGSDDINVVTCAAGILSNLTCNNYKNKMMVCQVGGIEALVRTVLRAG |
| | | DREDITEPAICALRHLTSRHQEAEMAQNAVRLHYGLPVVVKLLHPPSHWPLIKATVGL |
| | | IRNLALCPANHAPLREQGAIPRLVQLLVRAHQDTQRRTSMGGTQQQFVEGVRMEEIV |
| | | EGCTGALHILARDVHNRIVIRGLNTIPLFVQLLYSPIENIQRVAAGVLCELAQDKEAAE |
| | | AIEAEGATAPLTELLHSRNEGVATYAAAVLFRMSEDKPQDYKKRLSVELTSSLFRTEP |
| | | MAWNETADLGLDIGAQGEPLGYRQDDPSYRSFHSGGYGQDALGMDPMMEHEMGGH |
| | | HPGADYPVDGLPDLGHAQDLMDGLPPGDSNQLAWFDTDL |
| 14 | WT | MNIQEQGFPLDLGASFTEDAPRPPVPGEEGELVSTDPRPASYSFCSGKGVGIKGETSTA |
| | AXIN1 | TPRRSDLDLGYEPEGSASPTPPYLKWAESLHSLLDDQDGISLFRTFLKQEGCADLLDF |
| | | WFACTGFRKLEPCDSNEEKRLKLARAIYRKYILDNNGIVSRQTKPATKSFIKGCIMKQL |
| | | IDPAMFDQAQTEIQATMEENTYPSFLKSDIYLEYTRTGSESPKVCSDQSSGSGTGKGIS |
| | | GYLPTLNEDEEWKCDQDMDEDDGRDAAPPGRLPQKLLLETAAPRVSSSRRYSEGREF |
| | | RYGSWREPVNPYYVNAGYALAPATSANDSEQQSLSSDADTLSLTDSSVDGIPPYRIRK |
| | | QHRREMQESVQVNGRVPLPHIPRTYRVPKEVRVEPQKFAEELIHRLEAVQRTREAEEK |
| | | LEERLKRVRMEEEGEDGDPSSGPPGPCHKLPPAPAWHHFPPRCVDMGCAGLRDAHEE |
| | | NPESILDEHVQRVLRTPGRQSPGPGHRSPDSGHVAKMPVALGGAASGHGKHVPKSGA |
| | | KLDAAGLHHHRHVHHHVHHSTARPKEQVEAEATRRAQSSFAWGLEPHSHGARSRGY |
| | | SESVGAAPNASDGLAHSGKVGVACKRNAKKAESGKSASTEVPGASEDAEKNQKIMQ |
| | | WIIEGEKEISRHRRTGHGSSGTRKPQPHENSRPLSLEHPWAGPQLRTSVQPSHLFIQDPT |
| | | MPPHPAPNPLTQLEEARRRLEEEEKRASRAPSKQRYVQEVMRRGRACVRPACAPVLH |
| | | VVPAVSDMELSETETRSQRKVGGGSAQPCDSIVVAYYFCGEPIPYRTLVRGRAVTLGQ |
| | | FKELLTKKGSYRYYFKKVSDEFDCGVVFEEVREDEAVLPVFEEKIIGKVEKVD |
| 15 | WT | MSSAMLVTCLPDPSSSFREDAPRPPVPGEEGETPPCQPGVGKGQVTKPMPVSSNTRRN |
| | AXIN2 | EDGLGEPEGRASPDSPLTRWTKSLHSLLGDQDGAYLFRTFLEREKCVDTLDFWFACN |
| | | GFRQMNLKDTKTLRVAKAIYKRYIENNSIVSKQLKPATKTYIRDGIKKQQIDSIMFDQA |
| | | QTEIQSVMEENAYQMFLTSDIYLEYVRSGGENTAYMSNGGLGSLKVVCGYLPTLNEE |
| | | EEWTCADFKCKLSPTVVGLSSKTLRATASVRSTETVDSGYRSFKRSDPVNPYHIGSGY |
| | | VFAPATSANDSEISSDALTDDSMSMTDSSVDGIPPYRVGSKKQLQREMHRSVKANGQ |
| | | VSLPHFPRTHRLPKEMTPVEPATFAAELISRLEKLKLELESRHSLEERLQQIREDEEREG |
| | | SELTLNSREGAPTQHPLSLLPSGSYEEDPQTILDDHLSRVLKTPGCQSPGVGRYSPRSRS |
| | | PDHHHHHHSQYHSLLPPGGKLPPAAASPGACPLLGGKGFVTKQTTKHVHHHYIHHHA |
| | | VPKTKEEIEAEATQRVHCFCPGGSEYYCYSKCKSHSKAPETMPSEQFGGSRGSTLPKR |
| | | NGKGTEPGLALPAREGGAPGGAGALQLPREEGDRSQDVWQWMLESERQSKPKPHSA |
| | | QSTKKAYPLESARSSPGERASRHHLWGGNSGHPRTTPRAHLFTQDPAMPPLTPPNTLA |
| | | QLEEACRRLAEVSKPPKQRCCVASQQRDRNHSATVQTGATPFSNPSLAPEDHKEPKKL |
| | | AGVHALQASELVVTYFFCGEEIPYRRMLKAQSLTLGHFKEQLSKKGNYRYYFKKASD |
| | | EFACGAVFEEIWEDETVLPMYEGRILGKVERID |
| 16 | WT | MAAASYDQLLKQVEALKMENSNLRQELEDNSNHLTKLETEASNMKEVLKQLQGSIE |
| | APC | DEAMASSGQIDLLERLKELNLDSSNFPGVKLRSKMSLRSYGSREGSVSSRSGECSPVP |
| | | MGSFPRRGFVNGSRESTGYLEELEKERSLLLADLDKEEKEKDWYYAQLQNLTKRIDS |
| | | LPLTENFSLQTDMTRRQLEYEARQIRVAMEEQLGTCQDMEKRAQRRIARIQQIEKDIL |
| | | RIRQLLQSQATEAERSSQNKHETGSHDAERQNEGQGVGEINMATSGNGQGSTTRMDH |
| | | ETASVLSSSSTHSAPRRLTSHLGTKVEMVYSLLSMLGTHDKDDMSRTLLAMSSSQDSC |
| | | ISMRQSGCLPLLIQLLHGNDKDSVLLGNSRGSKEARARASAALHNIIHSQPDDKRGRR |
| | | EIRVLHLLEQIRAYCETCWEWQEAHEPGMDQDKNPMPAPVEHQICPAVCVLMKLSFD |
| | | EEHRHAMNELGGLQAIAELLQVDCEMYGLTNDHYSITLRRYAGMALTNLTFGDVAN |
| | | KATLCSMKGCMRALVAQLKSESEDLQQVIASVLRNLSWRADVNSKKTLREVGSVKA |
| | | LMECALEVKKESTLKSVLSALWNLSAHCTENKADICAVDGALAFLVGTLTYRSQTNT |
| | | LAIIESGGGILRNVSSLIATNEDHRQILRENNCLQTLLQHLKSHSLTIVSNACGTLWNLS |
| | | ARNPKDQEALWDMGAVSMLKNLIHSKHKMIAMGSAAALRNLMANRPAKYKDANIM |
| | | SPGSSLPSLHVRKQKALEAELDAQHLSETFDNIDNLSPKASHRSKQRHKQSLYGDYVF |
| | | DTNRHDDNRSDNFNTGNMTVLSPYLNTTVLPSSSSSRGSLDSSRSEKDRSLERERGIGL |
| | | GNYHPATENPGTSSKRGLQISTTAAQIAKVMEEVSAIHTSQEDRSSGSTTELHCVTDER |
| | | NALRRSSAAHTHSNTYNFTKSENSNRTCSMPYAKLEYKRSSNDSLNSVSSSDGYGKR |
| | | GQMKPSIESYSEDDESKFCSYGQYPADLAHKIHSANHMDDNDGELDTPINYSLKYSDE |
| | | QLNSGRQSPSQNERWARPKHIIEDEIKQSEQRQSRNQSTTYPVYTESTDDKHLKFQPHF |
| | | GQQECVSPYRSRGANGSETNRVGSNHGINQNVSQSLCQEDDYEDDKPTNYSERYSEE |
| | | EQHEEEERPTNYSIKYNEEKRHVDQPIDYSLKYATDIPSSQKQSFSFSKSSSGQSSKTEH |
| | | MSSSSENTSTPSSNAKRQNQLHPSSAQSRSGQPQKAATCKVSSINQETIQTYCVEDTPIC |
| | | FSRCSSLSSLSSAEDEIGCNQTTQEADSANTLQIAEIKEKIGTRSAEDPVSEVPAVSQHP |
| | | RTKSSRLQGSSLSSESARHKAVEFSSGAKSPSKSGAQTPKSPPEHYVQETPLMFSRCTS |
| | | VSSLDSFESRSIASSVQSEPCSGMVSGIISPSDLPDSPGQTMPPSRSKTPPPPPQTAQTKR |
| | | EVPKNKAPTAEKRESGPKQAAVNAAVQRVQVLPDADTLLHFATESTPDGFSCSSSLSA |
| | | LSLDEPFIQKDVELRIMPPVQENDNGNETESEQPKESNENQEKEAEKTIDSEKDLLDDS |
| | | DDDDIEILEECIISAMPTKSSRKAKKPAQTASKLPPPVARKPSQLPVYKLLPSQNRLQPQ |
| | | KHVSFTPGDDMPRVYCVEGTPINFSTATSLSDLTIESPPNELAAGEGVRGGAQSGEFEK |
| | | RDTIPTEGRSTDEAQGGKTSSVTIPELDDNKAEEGDILAECINSAMPKGKSHKPFRVKK |
| | | IMDQVQQASASSSAPNKNQLDGKKKKPTSPVKPIPQNTEYRTRVRKNADSKNNLNAE |
| | | RVFSDNKDSKKQNLKNNSKVFNDKLPNNEDRVRGSFAFDSPHHYTPIEGTPYCFSRND |
| | | SLSSLDFDDDDVDLSREKAELRKAKENKESEAKVTSHTELTSNQQSANKTQAIAKQPI |
| | | NRGQPKPILQKQSTFPQSSKDIPDRGAATDEKLQNFAIENTPVCFSHNSSLSSLSDIDQE |
| | | NNNKENEPIKETEPPDSQGEPSKPQASGYAPKSFHVEDTPVCFSRNSSLSSLSIDSEDDL |
| | | LQECISSAMPKKKKPSRLKGDNEKHSPRNMGGILGEDLTLDLKDIQRPDSEHGLSPDSE |
| | | NFDWKAIQEGANSIVSSLHQAAAAACLSRQASSDSDSILSLKSGISLGSPFHLTPDQEEK |
| | | PFTSNKGPRILKPGEKSTLETKKIESESKGIKGGKKVYKSLITGKVRSNSEISGQMKQPL |
| | | QANMPSISRGRTMIHIPGVRNSSSSTSPVSKKGPPLKTPASKSPSEGQTATTSPRGAKPS |
| | | VKSELSPVARQTSQIGGSSKAPSRSGSRDSTPSRPAQQPLSRPIQSPGRNSISPGRNGISPP |
| | | NKLSQLPRTSSPSTASTKSSGSGKMSYTSPGRQMSQQNLTKQTGLSKNASSIPRSESAS |
| | | KGLNQMNNGNGANKKVELSRMSSTKSSGSESDRSERPVLVRQSTFIKEAPSPTLRRKL |
| | | EESASFESLSPSSRPASPTRSQAQTPVLSPSLPDMSLSTHSSVQAGGWRKLPPNLSPTIE |
| | | YNDGRPAKRHDIARSHSESPSRLPINRSGTWKREHSKHSSSLPRVSTWRRTGSSSSILSA |
| | | SSESSEKAKSEDEKHVNSISGTKQSKENQVSAKGTWRKIKENEFSPTNSTSQTVSSGAT |
| | | NGAESKTLIYQMAPAVSKTEDVWVRIEDCPINNPRSGRSPTGNTPPVIDSVSEKANPNI |
| | | KDSKDNQAKQNVGNGSVPMRTVGLENRLNSFIQVDAPDQKGTEIKPGQNNPVPVSET |
| | | NESSIVERTPFSSSSSSKHSSPSGTVAARVTPFNYNPSPRKSSADSTSARPSQIPTPVNNN |
| | | TKKRDSKTDSTESSGTQSPKRHSGSYLVTSV |
| 17 | WT | MASSSGSKAEFIVGGKYKLVRKIGSGSFGDIYLAINITNGEEVAVKLESQKARHPQLLY |
| | CSNK1A1 | ESKLYKILQGGVGIPHIRWYGQEKDYNVLVMDLLGPSLEDLFNFCSRRFTMKTVLML |
| | | ADQMISRIEYVHTKNFIHRDIKPDNFLMGIGRHCNKLFLIDFGLAKKYRDNRTRQHIPY |
| | | REDKNLTGTARYASINAHLGIEQSRRDDMESLGYVLMYFNRTSLPWQGLKAATKKQ |
| | | KYEKISEKKMSTPVEVLCKGFPAEFAMYLNYCRGLRFEEAPDYMYLRQLFRILFRTLN |
| | | HQYDYTFDWTMLKQKAAQQAASSSGQGQQAQTPTGKQTDKTKSNMKGF |
| 18 | WT | MSGRPRTTSFAESCKPVQQPSAFGSMKVSRDKDGSKVTTVVATPGQGPDRPQEVSYT |
| | GSK3B | DTKVIGNGSFGVVYQAKLCDSGELVAIKKVLQDKRFKNRELQIMRKLDHCNIVRLRY |
| | | FFYSSGEKKDEVYLNLVLDYVPETVYRVARHYSRAKQTLPVIYVKLYMYQLFRSLAY |
| | | IHSFGICHRDIKPQNLLLDPDTAVLKLCDFGSAKQLVRGEPNVSYICSRYYRAPELIFGA |
| | | TDYTSSIDVWSAGCVLAELLLGQPIFPGDSGVDQLVEIIKVLGTPTREQIREMNPNYTE |
| | | FKFPQIKAHPWTKDSSGTGHFTSGVRVFRPRTPPEAIALCSRLLEYTPTARLTPLEACA |
| | | HSFFDELRDPNVKLPNGRDTPALFNFTTQELSSNPPLATILIPPHARIQAAASTPTNATA |
| | | ASDANTGDRGQTNNAASASASNST |

TABLE 4
Patient mutations sequences

| SEQ ID | | | SEQ ID | | |
|---|---|---|---|---|---|
| NO | Name | DNA Sequence | NO | Name | mRNA Sequence |
| 19 | Patient | CAGTCTTACCTGGATTCTG | 20 | | CAGUCUUACCUGGAUUCUGG |
| | PT4 | GCATCCACAGCGGAGCCA | | | CAUCCACAGCGGAGCCACCAC |
| | CTNNB1 | CCACCACCGCCCCTTATCT | | | CACCGCCCCUUAUCUGAGCGG |
| | p.S45Y | GAGCGGCAAGGGAAACCC | | | CAAGGGAAACCCAGAGGAGG |
| | | AGAGGAGGAGGATGTGGA | | | AGGAUGUGGAUACAUCC |
| | | TACATCC | | | |
| 21 | Patient | CCTGACAGGAAGGCCGCC | 22 | | CCUGACAGGAAGGCCGCCGU |
| | PT1 | GTGTCCCACTGGCAGCAG | | | GUCCCACUGGCAGCAGCAGA |
| | CTNNB1 | CAGAGCTATCTGGGCAGC | | | GCUAUCUGGGCAGCGGCAUC |
| | D32G | GGCATCCACTCTGGCGCCA | | | CACUCUGGCGCCACAACCACC |
| | | CAACCACCGCACCATCTCT | | | GCACCAUCUCUGUCUGGG |
| | | GTCTGGG | | | |
| 23 | Patient | GAGCAGCAGTCTCTGTCTA | 24 | | GAGCAGCAGUCUCUGUCUAG |
| | PT6 | GCGACGCCGATACCCTGTC | | | CGACGCCGAUACCCUGUCUC |
| | AXIN1 | TCTGACCGACTCTAGCGTG | | | UGACCGACUCUAGCGUGGAC |
| | p.G342fs | GACCCACCTATCCAGGAC | | | CCACCUAUCCAGGACCCA |
| | | CCA | | | |
| 25 | Patient | CCTGATCGGAAAGCCGCA | 26 | | CCUGAUCGGAAAGCCGCAGU |
| | PT2 | GTGAGCCACTGGCAGCAG | | | GAGCCACUGGCAGCAGCAGU |
| | CTNNB1 | CAGTCCTACCTGGGCAGC | | | CCUACCUGGGCAGCGGAAUC |
| | p.D32G | GGAATCCACTCTGGAGCC | | | CACUCUGGAGCCACAACAAC |
| | | ACAACAACCGCACCATCT | | | CGCACCAUCUCUGUCUGGC |
| | | CTGTCTGGC | | | |
| 27 | Patient | CAGTCCTATCTGGACAGCG | 28 | | CAGUCCUAUCUGGACAGCGG |
| | PT3 | GCATCCACTCCGGCGCAAC | | | CAUCCACUCCGGCGCAACAAC |
| | CTNNB1 | AACCACAGCACCTCCACTG | | | CACAGCACCUCCACUGUCUGG |
| | p.S45P | TCTGGAAAGGGAAATCCT | | | AAAGGGAAAUCCUGAGGAGG |
| | | GAGGAGGAGGATGTGGAT | | | AGGAUGUGGAUACAUCC |
| | | ACATCC | | | |
| 29 | Patient | TCTTATCTGGATTCCGGAA | 30 | | UCUUAUCUGGAUUCCGGAAU |
| | PT5 | TTCACTCCGGCGCCACCAC | | | UCACUCCGGCGCCACCACCAC |
| | CTNNB1 | CACAGCACCCTTCCTGTCT | | | AGCACCCUUCCUGUCUGGAA |
| | p.S45F | GGAAAAGGAAATCCTGAG | | | AAGGAAAUCCUGAGGAGGAG |
| | | GAGGAGGACGTGGACACA | | | GACGUGGACACAAGCCAG |
| | | AGCCAG | | | |

TABLE 5
Patient Amino Acid Mutation Sequences

| SEQ ID | | |
|---|---|---|
| NO | Name | Protein Sequence |
| 31 | Patient PT4 | QSYLDSGIHSGATTTAPYLSGKGNPEE |
| | CTNNB1 | EDVDTS |
| | p.S45Y | |
| 32 | Patient PT1 | PDRKAAVSHWQQQSYLGSGIHSGATTT |
| | CTNNB1 | APSLSG |
| | D32G | |
| 33 | Patient PT6 | EQQSLSSDADTLSLTDSSVDPPIQDP |
| | AXIN1 | |
| | p.G342fs | |
| 34 | Patient PT2 | PDRKAAVSHWQQQSYLGSGIHSGATTT |
| | CTNNB1 | APSLSG |
| | p.D32G | |
| 35 | Patient PT3 | QSYLDSGIHSGATTTAPPLSGKGNPEE |
| | CTNNB1 | EDVDTS |
| | p.S45P | |
| 36 | Patient PT5 | SYLDSGIHSGATTTAPFLSGKGNPEEE |
| | CTNNB1 | DVDTSQ |
| | p.S45F | |

TABLE 6
DNA Insert Sequences into pGX0001

| SEQ ID | | |
|---|---|---|
| NO | Name | DNA Insert Sequence |
| 37 | Patient | ATGGATTGGACATGGATTCTCTTTCTCGTTGCAGCAGCCACACGCGTTCACAGTCAG |
| | PT4 | TCTTACCTGGATTCTGGCATCCACAGCGGAGCCACCACCACCGCCCCTTATCTGAGC |
| | | GGCAAGGGAAACCCAGAGGAGGAGGATGTGGATACATCCAGAGGCCGCAAGCGG |
| | | AGATCTCACCTGGTGTCCAGAAAGTCCATCTCTGCCGCCCTGGAGCACAAGGTGAG |
| | | CGCCCTGCTGCCCCCCGCCGAGCAGACCGGAACATGGAAGCTGAAAACCAGAGGC |
| | | AGGAAGAGAAGATCCGACCTGCGGTCCCGCATCGAGGTGCTGAAGAGGAAGGTGA |
| | | TCGAGAAGCTGCAGCACATCCAGCTGCTGCAGAAGAATGTGCGGGCACAGCTGGT |
| | | GGACCGGGGAAGGAAGCGGCGCAGCAGCAAGGACCTGCTGCGGCAGCTGCCTGCC |
| | | TCCAACTTTAACTCTCTGCGCTTCCTGATCGTGCACCTGAAAAGAGTGGTGGATCAC |
| | | GCCGAGGAGAATCGGGGCAGAAAGCGCCGCTCCACCCTGCTGCTGGATGGCAACC |
| | | CTTTCAGAGTGCCCCAGGCCGCAATCCTGATGAAGGGCACCGCCGCAATCCTGGAG |
| | | TACCTGAGGGACAGAATCCCTAGGGGCAGGAAGCGGCGGTCCTCCATCCTGGAGA |
| | | GCAGCTCCGCCTTTCCCGATAATGCCGCCCGCAGGGAGGTGGAGAGCCCCCCAGCA |
| | | GTCTGTCCATCTGACGGCTGCACATGGAAGAGAGGACGCAAGCGGAGATCCGGGC |
| | | AGCCATACTTCGAGGCATTCAAGAAGAAGATGCCCATCGCATTCGTGGCAAAGGGC |
| | | ATCAATAAGCTGCTGAACAAGCTGTTTCTGATCAACGAGAGGGGCCGGAAGCGCA |
| | | GGAGCATGGAGGATCTGCAGGACATGTTTATCGTGCACACAATCGAGGAGATCGA |
| | | GGGCCTGATTGCAGCCCACGATCAGTTCAAGAGCACCCTGCCAGACGCCAGAGGCC |
| | | GGAAGAGGCGCAGCATGGGCGGCCAGACCGCCCTGAATTGCGGCGAGGAGCTGTT |
| | | TAAGAGAGGCGTGCTGAAGGAGTACGGCGTGAAGGTGCTGGGCACCTCTGTGGAG |
| | | AGCCGCGGCAGGAAGCGGCGCTCCGCCAAGGGCGTGAACGGCTCTAGCCAGGCCC |
| | | CTACCACCGGAAAGTACTGTCGCCTGTGCGACATCCAGTTTAATAATCTGAGCAAC |
| | | TTTATCACCCACAGAGGACGGAAGCGGCGGAGCCACATGAAGCTGAAAGTGCAGG |
| | | GAATGGAGTGTCTGGATGGCTGTTATGTCCACAGCAGCATCGTGCCAACATTCCAT |
| | | AGAAGATGCACATGGCTGCTGCGCGGCAGAAAAAGGAGATCTCAGGCAAGCAGAG |
| | | GCGAGGTGCCAAGCGGCGCCTACGGCTACAGCTATATACCTTCCGGAGCCTATGTG |
| | | TACCCCCCACCTGTGGCCAACGGCATGTATAGGGGCAGGAAGAGAAGGAGCTATT |
| | | CCAGCCTGAGATCTATCGTGGTGGCCAATTACGAGGAGTCCATCAAGATGCCAATC |
| | | AGCGAGCCAGCCCCTGGCAAGAAGAAGTCCCAGATCCAGCGCGGCAGGAAGAGGC |
| | | GCTCTGACCTGCAGGTGAGCGCCAAGATCGGCTTCCTGAAGCACCACGAGAATAAG |
| | | ACATACCTGGCCGTGGGCTCTATGAAAACCCTGATGCTGAACGTGAGCAGGGGCAG |
| | | AAAGCGCAGGTCCGAGCTGATCCTGCCTAGCGGCGCCAGGGTGGGCCACAGGAGC |
| | | CTGAGGAGATACTATAAGCAGAGGTTCGGCCTGTCTAGAGCCGTGGCCGTGGCCAA |
| | | GCGGGGACGCAAGAGGAGGTCTATGCTGGGCGAGCAGGGATTCCAGTCCCTGATG |
| | | GAAACCGTGGATACAGGCATCGTGACACAGCTGCAGGAGTTTCTGCAGGGATTCAG |
| | | GGGCCGCAAGAGGAGGTCCCGCCAGATCTGGTGGGAGAGACTGCAGGGCTGCCAG |
| | | CACATCGTGGAGGATTGGCAGAAGATCCTGATGGTGCGCTCCCTGGTGGTGAGCCC |
| | | CCACGAGAGAGGACGGAAGCGCAGGTCCAGCACCACCGGATCTAACTACTACGTG |
| | | AGAATCCTGTCTACAATCGATGGCGAGCTGCTGAAGCCAAATGCCAGCGTGGCCCT |
| | | GCACAAGCACTCCAACAGGGGCCGCAAGCGCAGGAGCGCCAACGAGCTGTGCGAG |
| | | GTGAACAGAAAGGGCTGCCCAAGCGCCGATCCATGTCTGCCTTACTTCTGCGTGCA |
| | | GGGCTGTAAGCTGGGCGAGGCCTCCCGGGGACGCAAGCGGAGGTCTTTTGAGGATT |
| | | GCACATCTGAGAACACCCTGATCAAGTATATGACCGATCGCATCCTGCTGAGGGAG |
| | | TCTCTGCGCGAGGCAGACCTGGACCACTATAGCCGGGGAAGGAAAAGGAGAAGCT |
| | | GGATCAGACCACTGGCATCCGGCAGCTACCCTAGCCGCAAGGCAGGAGCCACACT |
| | | GGTGGTGTACAAGGATCTGCTGGTGCTGTTCGGGGGCTGGACACGCGGACGCAAGA |
| | | GAAGGAGCTCCTCCGACTACTCCACATCCGAGATGCTGGTGAACGTGGGAAACCTG |
| | | CCCCTGGACGAGTTTTATCCCGCCGTGAGCATGGTGGCCCTGATGCGCATCCGGGG |
| | | CCGCAAGAGAAGATCCTGCGAGAGCCCCACAGCACACTGTAATGTGCTGAATTGG |
| | | GAGCAGGTGCACAGACTGGACGGCATCCTGTCTGAGACAATCCCAATCCACGGAA |
| | | GGGGAAGGGGGAGGAAGAGAAGGTCCAATAAGCAGAATAAGACCCCACTGGATA |
| | | AGTCTACATCCGGGGTGTCTGAGATCCTGCTGAAAACCCAGATGAAAATGAGCCTG |
| | | AAGTGCCTGGCCGCCCGCGGACGGAAGAGAAGGTCTTACGTGTGCGGCGGCATGTT |
| | | CTTCTTTGTGAGCTTCGAGGGGAAGATCGTGATGCACAAGATCATCTATATGGTGCT |
| | | GTTCCTGTTTTGTGTGGCACTGAGAGGCAGAAAGAGGAGGTCCCTGCCTACCCCAG |
| | | GGTCTTGGTGGGAGCAGCTGACCCAGGCATCTCGGGCCTACGCAAGCGGAGGCAC |
| | | CGAGGGCTTTCCTCTGAGCCGGTGGGCCCCCAGGGGCAGAAAGAGAAGGTCTTATT |
| | | CCACCCTGTCCCTGTTTGACAAGTACAAGGGCAAGTCCGTGGATACCATCCGGAGC |
| | | TCCGTGATCCCCCGCCACGGCCTGCAGTCCCTGGGCAAAAGAGGCAGAAAGAGAA |
| | | GAAGCATGAACATCTCTCTGGTGTGGTGCCTGCTGGTGCTGTCCTTCGCCATCAGGA |
| | | GGTGGTGGCGCGGCAGGAAGCGCCGGAGCAGGGTGGAGCTGCAGCAGGAGGTGG |
| | | AGAAGCTGGCACACGAGAATAGCTCCATGCGGCTGGAGCTGGATGCCCTGAGGTC |
| | | CAAATATGAGGCACTGCAGAGGGGGAGAAAGAGAAGGTCCAGCCCTCAGAGAGA |
| | | GCCAAGAGGCGGCCAGCTGAGAACCCCAAGAATGTGGCCTTCTTGTTCCAGAAGCC |
| | | TGGAGTCTCTGAGAGTGGGAGCCAAGCCCCGGGGAAGGAAGAGACGCTCCTTCAC |
| | | ACTGTGGAACGAGAGCACCCACTATCAGATCCTGCTGACCTCTTTTCCCCACGTGG |
| | | AGAATCACTCTTGTTTTGAGCACATGCACCACATCCCAAGAGGCAGAAAGCGGCGG |
| | | TCTGATTTTGTGGTGGAGGCCATCGGAACCGAGGTGGGCACCCTGGGATTCTCCAT |
| | | CGAAAGACCTTCCCAGGCCAAGATCGAGTGCGACGACAAGGGCGATCGGGGCAGG |
| | | AAGCGCAGATCCAACGTGATCGACAGCATGCTGTTCGTGTTCGGATATAGAGCCCA |
| | | GAAGAATAAGATCCGCGGAAGGAAGAGGCGGTCTATGATGGGCAGCGCAAGGGTG |
| | | GCAGAGCTGCTGCTGCTGCACGGAGCAGAGCCCAACTGTACCGACCCTGCAACACT |
| | | GACCAGACCAGTGCACGACGCAAGAGGAAGGAAGAGAAGAAGCCCCCCAGGCCT |
| | | GGACAAGAGACTGCTGCCAGAGACACTGGGCCCCTGCTACTCCAATTCTCAGCCTG |
| | | TGTGGCTGTGTCTGACACCACGGCAGCCACTGAGAGGCAGGAAAAGAAGATCTTCC |
| | | TTCCTGCTGAGCCCTAATAATGGCAACCTGGAGGCCACATCTAATATCGAGTACGC |
| | | ACACGTGCCCCACCTGTCTCCTGCCGTGATCCCTACACGGAGGGGCCGGAAGAGGA |
| | | GGTCTGAGGAGGATGAGGAGGACACCTATTATACAAAGGACCTGCCCATCCACAC |
| | | CTGCAGCTACTGCGGAATCCACGACCCAGCCTGCGTGGTGTATTGTAACCGCGGCA |
| | | GGAAGAGACGGAGCCTGATCTCTAGGAGGGATGCCGAGGTGGTGCTGACAAGCAG |
| | | GGAGCTGGGCAGCCTGGCCCTGAACCAGTCTACAGGCCTGCCTACCCTGACCCTGC |
| | | CAAGAGGCCGGAAGCGGAGGTCTGACTTCTCTTCTAACGACTCCTCCACATGCACC |
| | | ATGGGCCTGGTGCTGACACTGTGGTCTGATACCCTGATCCACCTGGACGGCGATGG |
| | | AGGCTTTAGCAGAGGCAGAAAAAGGAGGTCCTCCAGCGCAGCCCCATCCGTGATG |
| | | GACCTGAGCTCCCGGGCATCTGCCGCACTGCACCCAGGCATCCCCTCCCCTAATTGT |
| | | ATGCAGTCTCCCATCCGC |
| 38 | Patient | ATGGATTGGACCTGGATTCTCTTTCTTGTTGCTGCTGCCACTCGCGTTCACAGTCCT |
| | PT1 | GACAGGAAGGCCGCCGTGTCCCACTGGCAGCAGCAGAGCTATCTGGGCAGCGGCA |
| | | TCCACTCTGGCGCCACAACCACCGCACCATCTCTGTCTGGGAGAGGCAGGAAACGG |
| | | AGAAGCGGCCAGTCTCCTACACTGCTGACATCTGCCTGTTTCTTCCCATCTGCAGCC |
| | | TTTACAGCCGAGTTTCTGTTTAGCCTGGGCTCCCTGTGTCAGCACCTGCGGGGCCGG |
| | | AAGAGAAGATCTCGCCTGGCCAGCGCACCTCCACAGAAACAGAAGCAGAAGCTGG |
| | | GCGAGAGGCTGTTCCCCCTGATCCAGGCAATGCACCCAACACTGGCCGGCAAAATC |
| | | AGGGGCAGAAAGAGGAGGTCTTGCGGCCACCTGTATGCCTACGATTGGATCTCTAT |
| | | CCCAGTGGTGTACACCCAGGTGGTGACCGTGGCCGTGTATAGCTTTTTTCTGACCTG |
| | | CCTGGTGAGGGGCAGGAAGAGACGCAGCGACTGTATGTCCGAGGATGGATATAAG |
| | | CTGGATAGGATCAAGGGAGAGTTCGAGTTCCACAATGTGACCTTTCACTATCCCAG |
| | | CCGCCCCGAGGTGAAGAGAGGAAGGAAGCGGAGGTCTATGGCCAACGATTCCCCT |
| | | GCCAAGTCCCTGGTGGACATCGACCTGCCTTCCCTGAGGGACCCAGCCGGCATCTT |
| | | CGAGCTGGTGGAGGTGGTGGGAAATAGGGGCCGCAAGAGACGCAGCTGGCTGGGC |
| | | ATCCGGATCTCTGAGTCTCCAGAGCCAGGGCAGCGGACATTCACAACCTTTAAGTT |
| | | TTGCCAGCTGCCATTCTACAAGTATTCCGAGTATAGAGGCAGAAAGCGGAGGAGCC |
| | | CCCCCCTGGCAAGACCAATCCTGCCAAGGGAGCGCGGCGCAATGGATAGAATCGT |
| | | GGAATACCTGGTGGGAGACGGCCCTCAGAACAGATACGCACTGAGAGGCCGGAAA |
| | | AGGAGAAGCACCTGCGTGTCCCTGGATGACGGGCTGTACGAGTGTAGCTGTGCCCC |
| | | TGCCTATTCCGGAAAGGATTGTCAGAAGAAGGATGGACCTTGCGTGATCAACAGGG |
| | | GCAGGAAGCGCAGAAGCCTGCACGTGGGCCTGTGTTCCGGCCCCTGTGAGATGGCC |
| | | GAGCAGCGGTGCTGCGTGGACTACGCCAAGAGGGGCACCGCCGGGTGTAAGAAGT |
| | | GTAAGAGGGGCCGGAAGCGGCGCTCCAGCTTCGAGAGCTACCTGAAAACAATGCC |
| | | ACCATACTTTCTGGGCCCACTGAAGAAGGCAGTGCGCATGATGGGAGCCCCAAACC |
| | | TGATCGCAGACAGCAGGGGCAGAAAGCGCCGCAGCCCTAAGTTCCACTCCTACGTG |
| | | TCTCTGCCATTCGGCTGCACAAGGGCCGTGGTGGAGTACAGGCTGCTGCAGGCCGC |
| | | ATACCTGGCCAAGCCCGGCGACAGAGGCCGGAAGCGCCGCTCCGGAGAGTACGTG |
| | | GATACAGGCAAGCTGATCGATAAGATCAATCTGCCTAATTTCCTGAAGGTGTATCT |
| | | GAACCACAAGCCACCTTTTGGCAACACCATGCGGGGCAGGAAAAGAAGATCTATG |
| | | TGCACCAACGCCAGAAGGGTGAGAAAGAGATGGCTGCCTAAGATCAACAGCATGC |
| | | TGCCCGAGGGAGTGGAGATGTACCGCACCGTGATGGGATCTAGAGGGAGAAAGAG |
| | | AAGAAGCCCAGACGGCCCACTGCTGCCATCTCCCGGCCTGCTGAGACTGCCAGGCG |
| | | GGCCTCGGCACGCCGTGTCTGGAGTGCCTGAGGTGTACCCCCCTGGCCCTAGAGGC |
| | | AGGAAGCGGCGGAGCGTGGGCACACAGGCCACCAAGTATAATAGCGAGGTGGTGG |
| | | ACAAGATGATCGAGGAGTTTCTGTCCAGCTTTGAGGAGAAGATCGAGAATCTGACA |
| | | GAGAGGGGACGGAAGCGCCGGAGCACAAACCTGCCTATCTTTAAGCTGAAGGAGA |
| | | GCTGCGTGCGCAGGAGATACTCTGATTTCAAGTGGCTGAAGAATGAGCTGGAGAGA |
| | | GACTCTAAGATCAGAGGCAGGAAAAGGCGCTCCCTGATCAGATGGTGTCTGGCACT |
| | | GCGCCCAAGCGACAGGCCAACCTTCGAGGAGATCCAGAAGCACCCATGGATGCAG |
| | | GACGTGCTGCTGCCCCAGGAGCGGGGCCGGAAGCGCAGATCCAAGTTTGAATGCG |
| | | GCTGGTGTAGCGGCGAGAGGAGGTGTACACTGCACCAGCACTGCACAAACCCAAG |
| | | CTCCCCATGGCTGGATTGGTCCAGCCACAACAGGGGACGGAAGAGACGGAGCCAC |
| | | GACCACACCGGCTTCCTGACCGAGTACGTGGCCACAAGGTGGTACAGCGCACCCGA |
| | | GATCATGCTGAATTCCAAAGGCTACACAAAGTCTATCGATAGGGGAAGGAAGCGC |
| | | CGCAGCTCCTCCGAGGCAACCGAGGTGAATCCAGAGTCTCTGGCAAAGGAGTGCCT |
| | | GGAGAAATCTCTGCTGCTGCTGAAGTTCCTGCCCACCGGCATCAGCTCCAGGGGAA |
| | | GGAAGAGGAGAAGCGTGAAGAAGGATAGATCCGCCTCTCACCTGGACCACAAACT |
| | | GGAGATCCGCCAGTGGAGGAGGTCCTTTCTGGTGTCCCTGTTTTTCTGCATCCCCGT |
| | | GCGCGGACGCAAGCGCAGGAGCCTGTTTACATTCGCCGTGGGAGTGAATATCTGCC |
| | | TGGGCTTTACCGCCTACAGGATCAAGAGAGCAGAGGGATGGGAGGAGGGCCCTCC |
| | | CACCGTGCTGCGGGGCAGGAAGAGGAGATCCATGAGCCAGGCCCCTGGCGCACAG |
| | | CTGTCTCCCCCAACCGTGTACCACGAGAGACAGAGGCTGGAGCTGTGTGCAAGGGG |
| | | CAGAAAGCGGCGCTCCATGTCTTCTAACAGCTTTCCCTACATCGAGCAGTCCGGAG |
| | | GAGGCGAGGCCACCGAGCTGGGCCAGGAGGCCACCAGGGGAAGGAAAAGAAGGT |
| | | CCCAGCCTTGTCGCAAGATGCTGCCTGATCGGTCCAGAGCCGCCAGATACAGGGCC |
| | | AATCAGAAGCTGGTGGAGTATATCGTGAAGGCAAAGGGAGCCGAGAGGGGCCGCA |
| | | AGCGGAGAAGCCCTGTCTTTCACGGCATGCTGGAGAGAGCACCAGCCGAGCCTTGC |
| | | TATCGGGCCCCAATGGAGAAGCTGTTTTATCTGCCACACGTGTGCTCTTATACCCGG |
| | | GGCCGCAAGCGGCGCTCCACCTACTCTTGCGTGGCCGAGAATATCCTGGGCACAGG |
| | | ACAGAGAGGCTCCGGCGCCGAGCTGGATGTGCAGTATCCCCCCAAGAAGGTGACC |
| | | ACCGTGAGAGGCAGGAAGCGCCGCTCCAGCCCCAGCGCAACAGAGGATTCCCTGC |
| | | AGCCTGCCACAGACCTGCTGAACAGGTCTGAGCTGCCCCAGAGCCAGAAGGCAAT |
| | | GCAGACCAAGGATGCCCGCGGCAGAAAGAGAAGGTCTGACCTGAGAAAGGTGCCC |
| | | GGCACAGACCCCGCCTTCGAGGTGTTTCCCCTGCACGATCACCAGTGTGTGCTGCG |
| | | GGGCCGCAAGCGCCGGAGCGACGCACTGGATTTCAAGAAGGATAAGGGCGCCTTC |
| | | TATCTGGTGTTCATCTGGACCATGACCAGAGGCAGGAAGAGAAGATCCGTGGATAA |
| | | CTACAAGCAGATCAAGGCCGACCTGGATCAGCTGAGGCTGCCTGTCGAGAAGTCCG |
| | | AGCTGTGGGTGGAGAAGAGCTCTAACTATGAGAATAGGGGCAGAAAGAGAAGATC |
| | | CAATGCCCCCGAGAATGGATATATGAAGTGCTCCAGCGAGGGCGACAACTACGGC |
| | | GCCACCTGCGAGTTCTCTTGCATCGGAGGCTATGAGCTGCAGGGCCGGGGCCGCAA |
| | | GAGAAGATCCGAGAGCGGAAGCAGCGGCAAGAGCTCCTCTTCCCTGGTGCGGGGA |
| | | GTCCACCTGAGCAGCTCCGGACCCGCCCTGCTGGCCGGCCTGGTGTCCCTGGACAG |
| | | GGGCCGGAAGAGGAGATCTTGGACAGAGAAGCGGGCCTCTTATGAGCTGGAGTTC |
| | | GCCAAGTCCACAACAAAGATCGCCGAGGCCGGCAAGGTGTCCATCCAGCAACAGT |
| | | CTCACATGAGAGGCAGAAAGCGCAGGAGCAAGATCCTGGCAAGGAAGAAGTCCAA |
| | | ACGGTCTGCACTGGAGAACTCCGAGGAGCACTCCACAAAGTACTCTAATTCCAACA |
| | | ACTCTGCCGGCTCCGGGAGAGGCAGAAAAAGAAGGTCCGCAGCAGCAGCCGCCGC |
| | | ACTGGCCGCAGCCCTGCTGCTGCTGAGGCGCGAGGACCCAGGACTGGGGGCCGGC |
| | | CCCAGCATGGCAGAGACAGAGGCCCTGCGCGGCAGAAAGAGGAGATCTCTGCCTT |
| | | CTTTTCCTCCTTTCGGCTCTATGAACTCTAACGCCGCAGGCAGCGTGAGCACCCAGG |
| | | CCAATACCGTGCAGAGCGGACAGCTGGGAGGACAGAGAGGCAGAAAGAGACGGT |
| | | CCAGCGGAGTGGTGCACCGGCTGGAGGTGGACGAGGACTTCGAGGAGCACAATGC |
| | | CGCAAAGGTGCATCTGATGGTGCACAATCTGGTGCCACCATTCCTGAGGGGCAGGA |
| | | AGCGGAGGAGCCTGCAGATCAAGCAGCAGGAGCTGTATAAGAACTTCCTGAGATT |
| | | CCAGGTGGAGGAGAAGAAGCAGCGGGAGGAGGCCGAGCGCGAGCGCCTGAGAAT |
| | | C |
| 39 | Patient | ATGGATTGGACATGGATTCTTTTTCTCGTTGCAGCAGCTACACGCGTTCATAGCGAG |
| | PT6 | CAGCAGTCTCTGTCTAGCGACGCCGATACCCTGTCTCTGACCGACTCTAGCGTGGA |
| | | CCCACCTATCCAGGACCCAAGGGGCAGAAAAAGGAGATCTGACCAGAACACCGAG |
| | | GATTACCTGTTTCAGGTGATCCTGGAGAAGCAGATCAGAATCCCCCACAGCCTGAG |
| | | CGTGAAGGCCGCCTCCGTGCTGAAGTCTAGAGGCCGGAAGAGAAGGAGCCTGAGA |
| | | GAGGATGGCCGGGAGCAGGAGATCCGCGACGGCGGCTCCCTGCGGATGATCCACT |
| | | TCGGCCTGGTGAGAGGCCGCAAGAGACGGAGCCTGCTGCAGCTGTGGCCAGTGCA |
| | | GCGGCTGACAGTGAAGAAGTCCACCGACAATCTGTTCAGAGAGCAGAAATCCTCCC |
| | | TGAACTACCTGCGCCAGAAGCGGGGCAGAAAGCGCCGGTCCCAGTGGCTGAGGGC |
| | | CGTGGCAGTGGAGAGCATCCACCGCTTCTGTATGCAGCCACAGCTGCTGAGATCCT |
| | | TCTGTCAGAGCTATGATATGAAGCAGCACAGAGGAAGGAAGCGGAGGAGCCTGGA |
| | | GGCCATCGTGCAGCTGTGGCGCATCCCATCCTTCGTGACCGAGCTGTATGTGAATT |
| | | ATGATTGTGATTATTATTGCTCCAATCTGTTCGAGGAGAGGGGGAGAAAGCGCAGA |
| | | TCCACCCCTCCCGGCCAGTCCCCAGGCTCCGCCCCCATCATCATCAACATCCACAG |
| | | AGCCCAGCTGACAAACCCAGAGGTGAAGTACAATTATACCGAGGACAGGGGACGG |
| | | AAGAGACGCTCTCCTTATGCCACAGTGGCACCCTCCACCCTGGCCCACCCTCAGGC |
| | | CCAGGTGCTGGCCAGGCAGCAGGCCCTGCAGCACGCCCAGACCCTGGCCCACGCC |
| | | AGAGGCAGAAAACGGAGAAGCCAGAGGGGCCTGCTGCAGCAGATCGGCGACGCA |
| | | CTGTCCAGCCAGAGGGGACGGGTCCCCCCAGCCGCACCCCCTGCACAGCCCAGGGT |
| | | GCCAGTGACCAGAGGCAGAAAGCGCAGGTCCATGTTTAGGTCTAAGAGATCCGGC |
| | | CTGGTGAGGAGGCTGTGGCGGTCCGGCGTGGTGCCCGATCGCGAGGAGGGAGGAA |
| | | GCGGCGGCGGCGGGGGAGGCCGCGGCAGAAAGAGGCGGTCCCTGGAGATCAAGGT |
| | | GAGCCCACCCGAGGGCGCAGAGACACGCAAGGTGAGAGAGATCGGCCCTCTGTGC |
| | | GGACGCAGAAGGCCACGGGTGAGAAAGTCCAGGGGCAGGAAGAGAAGGTCTACC |
| | | CCTAACTTTATCATCCCAGCCCAGAGAGCCGAGCCTATGAGGATCGTGCGCCAGCC |
| | | TATGCCACCCCCCGGGGACCTGGAGCCTCCTTTCCAGCCTAGAGGCAGAAAGCGGC |
| | | GCTCTGGCACAGGCGCCCTGGGCCTGCTGCTGCTGTTCCTGCTGGGCTATGGACTG |
| | | AGGAGAAGGCCTCAGAAGGCCGAGGAGCCTGCACCTCAGGCAGGCGGCCGGGGAC |
| | | GGAAGAGAAGATCTGACAGCCACGTGTCCGAGGTGCTGCAGGAGAACTATCGGCT |
| | | GAAAAACAAGCTGGAGGGACTGATCAGCGAGAAGAATGAGCTGAAGATGAAGAG |
| | | CGAGAGGGGACGGAAGCGGAGATCTGTGTCTGCATACCAAGTGCAGAGCCCAAGC |
| | | TGGATGCAGCCTCAGCCATGTATCCTGCAGCACCCTGGCGCCGTGCTGACCCCTTCT |
| | | ATGGAGCACACCAGGGGCCGGAAACGGAGGAGCGGCAGGTTCAGCATCGTGAAGA |
| | | AGTGCATCCACAAGGCAACCAGGAAGTACGTGGCCGTGAAGTTCGTGTCCAAGAA |
| | | GATGAAAAAGAAGGAGCAGGCCCGCGGACGCAAGCGCCGGTCTGGCATCCTGCGC |
| | | GACCCAGGCTCTGAGATCGAGGACAGACAGTATAGAATCGATCTGCAGTCCATCAA |
| | | TATCGGAACCGCCCAGTGGCACCAGCTGAAGAGAGGAAGGAAGAGAAGGTCCGAC |
| | | GGAACACAGGTGCTGAAGATCTACGTGAAGCAGACAGAGTCCCACTACATCCTGGT |
| | | GTCTTGGAAGGTGAATTCTAACGTGATGACATCTAATCTGAGAGGCCGCAAGCGCC |
| | | GGAGCCCCCCAGGATACATCCCCGATGAGCTGCACCAGGTGGCCCGGAACGGCCT |
| | | GTTTACATCTATCAACTCTGAGGGCGAGTTCATCCCCGAGAGCATGGATAGGGGCA |
| | | GAAAGAGACGGAGCCCAAAGTTTCGGGGAGTGAAAATGATCCCTCCAGGCATTCA |
| | | CTTCCTGCACTATAGCAGCATGGACAAAGCCAATCCTAAGGAGGTGGGCCCACGCA |
| | | TGAGAGGCAGAAAGAGAAGATCCAATGTGAGCAGCGCCGAGGGATGGCACGTGAA |
| | | CGTGACCCTGTCTATCAGGCCCTCCACAGGCACCGGCGTGATGCTGGCACTGGTGA |
| | | GCGGAAATAACAGGGGCCGCAAGAGGAGATCTTGTCAGAGGTCTTTTAGGACCTTT |
| | | CAGGCCCTGAAGAAGCACCTGGAGGCCTCCCACCTGGAGCTGAGCGAGGCCGATA |
| | | TCCAGCAGCTGTACGGCGGCCGCGGCAGGAAGCGCAGATCCGACTCCGTGGACTTT |
| | | CGCCTGAGGGTGAGCGAGCCCAAGGCAGTGTTCGCCAAGGAGCAGCTGGCATGCA |
| | | GGGAGGTGCAGGCAGAGGTGGGCGCCTCTAGAGGCAGGAAGAGGCGCAGCGATAT |
| | | CAAGGATGGAAGATTTCCTTACGGATCTACCCAGGATTACCTGAAGCCAATCATCC |
| | | TGATCAAGCTGGTGCAGCTGGGCATGGCAAAGGATGACAGGGGCAGAAAGAGGCG |
| | | CTCCGAGAACTACCGGAACCTGGCCTTTCTGGGGATCGCACTGTCTAAGCCAGACC |
| | | TGATCACATTCCTGGAGCAGGGCAAGGAGCCCTGGAATATGAAGCAGAGGGGCAG |
| | | GAAGCGGAGATCCTGGAATAGCCTGGGCAAAATGTCTCGCGAGGAGGCCATGAGC |
| | | GCATACGTGACCGAGATGAAGCTGGTGGCCCAGAAGGTGATCGATACCGTGCCCCT |
| | | GAGAGGGCGGAAGCGCAGGTCCACATTTCAGTGCGTGGTGTCCCCCTCCGACGTGG |
| | | CAGTGGTGTGGTTCTGGGATGGAGCCCTGCTGCAGCCCAGCGAAAAGTTTGCCATC |
| | | TCCCAGAGCAGGGGCAGAAAGCGGAGGTCCCACGTGGATCTGTTCGACCTGGAGA |
| | | GCGGAGAGTATCTGTGCCCACTGTGCAAGAGCCTGTACAACACAGTGATCCCAATC |
| | | ATCCCACTGCAGCCTCAGCGCGGCAGGAAGAGGAGATCTAGCGAGATCATCAGCA |
| | | AGCTGTACATCCCCAAGAGGAAGATCATCTCTCCAAGAAGCATCAAGGACGTGCTG |
| | | CCACCAGTGGAGGAGGCCGTGGATAGG |
| 40 | Patient | ATGGATTGGACTTGGATTCTCTTTCTCGTTGCCGCAGCTACACGCGTTCACTCACCT |
| | PT2 | GATCGGAAAGCCGCAGTGAGCCACTGGCAGCAGCAGTCCTACCTGGGCAGCGGAA |
| | | TCCACTCTGGAGCCACAACAACCGCACCATCTCTGTCTGGCCGGGGCAGGAAGAGG |
| | | AGGTCCGGCGATAACACAGGCGAGCAGGTGGCCGTGAAGTCCCCTAAGCCTGAGA |
| | | GCGGAGGCAATCACATCGCCGATCTGAAGAAGGAGATCGAGATCCTGAGGAGAGG |
| | | GAGAAAGCGGAGATCTAAGGATCACGGCGTGAACTCCTTTCTGGTGTACATGGCCT |
| | | TCAAGGACCTGTTTCAGCTGACAGATTGTCAGATCTACGAGGTGCTGTCCGTCATCC |
| | | GGAGAGGCAGAAAGCGGAGGTCTAACACAGACGGCAAAATCGAGTTCATCTCTAC |
| | | CATGGAGGGCTACACCTACCCAGTGTATGGCGTGCAGTGGCATCCTGAGAAGGCAC |
| | | CCTACGAGTGGAGGGGCCGCAAGAGGAGATCCAATAATAGCGTGCTGCAGAAGCA |
| | | GCAGCTGGAGCTGATGGATACCGTGGACAATCTGGTGAACCTGTGTACCAAGGAGG |
| | | GCGTGCTGCTGAAGGGAGGCCGCGGCAGAAAGAGACGCAGCGACTCCAACCGGGA |
| | | GAAGGACATGGGACTGTTCGAGGTGTTCTCTCAGCACCTGCCCACCACCGAGCCAG |
| | | TGGACAGCTCCGTGAGCTCTAGCATCAGCAGAGGCCGCAAGCGGCGCAGCCTGAA |
| | | GGCCGAGGTGTTCACAGAGATCCTGAATTACATCTATTCCAGCACAGTGATCGTGA |
| | | AGAGGCAGGAAACCGTGACCGACCTGGCCGCCGCCGGCAGAGGCAGAAAGAGAC |
| | | GGTCCCACCCAAATAACGTGGTGTCCATCAAGTACTGCAGCCACTCCGGGCTGATG |
| | | TTTAGCGTGTCCACCTCCTACATCAAGGTGTGGGATATCCGCGACAGCAGGGGCAG |
| | | AAAAAGAAGGTCCACAAAGAACAAGCGGAGAAAGGAGTATTTTCTGGCAGTGCAG |
| | | ATCCGGAGCCTGGAGGAAAAGATCAAGGTGAAGTCTACAGAGGTGGAGATCCTGG |
| | | AGAGAGGAAGGAAGCGCCGGTCCCTGGTGAAGCCACAGAAGGAGAATGAGCAGG |
| | | CCGAGAAGAAGAACATCTCTCTGGCCTTCTTTCTGTATGACCTGCTGTCCCTGATGG |
| | | ACAGAGGCTTTAGAGGACGGAAGAGGCGGTCTGTGGATATCGTGATCCTGGGCCTG |
| | | AGCTACGCCAACCTGCAGACCGGCAGCGGAGTGCTGAGCGATTCTCTGAGCGGCG |
| | | GAATGCAGCTGCTGCCAGATAGGGGCAGAAAGCGGCGCTCTATCACCAAAATCAC |
| | | CAGGAGACGCCACGAGAATCTGCCCCACGGCGTGGCAAGCGTGAAAGAGTGGTTC |
| | | AATTACGTCACCGCAACACGCAACGAGGAGAGAGGACGCAAGAGGCGGAGCGCCG |
| | | TGCTGAAGGTGGGATGGCTGTTTCCTCTGTCCGAGGTGCCTAACTTTACACTGCTGA |
| | | TGGACGGCTGTGGCTGCTGGAGGCTGAAGGAGGATCAGCGGGGCCGCAAGAGAAG |
| | | ATCTCTGGTGGCCGCCCTGCTGCTGGGCTCCGTGTGCTGCGGCTCCGCCCAGCTGCT |
| | | GTTCAATAAGACCAAAAGCGTGGAGTTCACCTTCAGGGGCCGGAAGAGGCGCTCC |
| | | AGCCACGTGTTTGGAACCAACACCAGCTCTCTGGAGCTGTTCCTGATGAACGGCAA |
| | | GATCAAGGGACCCTGTTGGCTGGAGGTGAAGTCTCCACAGCTGAGAGGAAGGAAG |
| | | AGAAGGAGCTCTCCCTACGATCACACACCAGGGATGGCAGGCTCTCTGGGCTACCA |
| | | CCCCTACGAGGCCCCCCTGGGAAGCTATCCATATGGCGACCCAGCCTATAGGAGGG |
| | | GCAGAAAGCGCAGATCCATGACAGGGTCCTCCCTGTCCCTGGCCCACCTGCTGATC |
| | | ATCAGCGGCCTGCTGTGCTATTCCGCAAGGGGCAGGAAGCGGCGGTCCGCCGAGG |
| | | AGGGAGACGAGCTGCAGCAGAGATGTCTGGACCTGGAGAGACACCTGATGCTGCT |
| | | GAGCGAGGAGAAGCAGTCCCTGGCCCAGGAGAATGCCAGGGGCAGGAAGCGCAG |
| | | ATCTACCATGTATCACGGAGTGCCTGTGGTGGGCATCCCTCTGTTCGGCGACCACTA |
| | | CGAGACAATGACCCGCGTGCAGGCCAAGGGAATGGGCATCCTGCTGAGGGGCAGG |
| | | AAGAGACGCTCCCAGCCTGCATCTCACATCCTGAATAGCGAGAAATTCCCTTTCTC |
| | | CAAGTACGAGGACTGCCTGGAGCAGCAGATGCCCAGCCTGCGCGAGTCTCCAATGC |
| | | GCGGCAGGAAGAGACGGTCTGTGGAGAAGAGCCCCGCAAAGAAGAAGGCCACCA |
| | | AGAAGGCCGTGGGCGCCGGAGCAGCCAAGAGAAAGGCCACCGGCCCACCCGTGAG |
| | | CGAGCTGATCCGCGGCAGAAAACGCAGATCCCTGCTGGCAGCCGGCCCATCCGCCG |
| | | CCGCCGCCAAGCCAAATATCCCTAAGGTGCTGCTGCCATTTACAAGGGCAACAAGA |
| | | GTGAACTTTACCCTGGAGAGGGGCAGAAAAAGGAGGAGCCTGGCACCCCAGGACA |
| | | TGAGAGTGTTTTACGCACACACCGCCAGGGCCCAGCTGCCCGTGTGGAACAATTGG |
| | | ACCCAGAGAGCAGCCCGCTATGAGTGCCGGGGCCGCAAGCGGAGGTCCCCTGCAC |
| | | CTCTGAGACGCCCCCTGGCAGTGCTGCACGCCCCAGTGCCAACAACCGCACTGGCA |
| | | AGACTGTCCAGGCCTCAGCGGGCATGTCCATCCTCTAGGGGCAGAAAGAGGCGCA |
| | | GCAAGCACTGGTTCGTGCTGACAGATTCCTCCCTGAAGTACTACAGAGACTCTACA |
| | | GCAGAGGGCGCCGACGAGCTGGATGGAGAGATCGATCTGCGCAGCAGAGGCAGGA |
| | | AGAGGCGGAGCGTGAAGTTTCTGAGGAAGCCAGGCGGAAATCTGGGAAAGGTGTA |
| | | TCACCCAGGCTCCATGCTGTCCCTGGCACCTACAAAGGGCCTGCTGAATGAGCCTA |
| | | GAGGCCGGAAGCGCAGGTCTGATTATCAGGTGTACCTGAACGCCAGCAAGGTGCC |
| | | CGGATTCGCCGATGATCCTACAGAGCTGTCCTGTCGGGTGGTGGATACAAAGAGCG |
| | | GCGAGGCACGCGGGAGGAAGAGGAGGAGCCAGCGGGATGATCCTGTGCTGGAGAC |
| | | CAGCCACCAGAACTTTAGGCGCTCCAGATATCAGGAGGCCGCATCTCCAAGGGAG |
| | | GCCCTGATCCGCCTGCGCCGGGGCAGGAAGCGGAGATCTCACCTGCTGAATTATGT |
| | | GTGGCCAAACGTGTTCGAGACCTCTCCACACGTGATCGAGGCAGTGATGGGCGCCC |
| | | TGGAGGGCCTGAGAGTGGCCATCGGCAGAGGCAGGAAACGCAGGAGCAGCAGGA |
| | | CATCTATGGACGATCTGAAGGCCTTCACATCCCTGAGCCTGTATATGAAGCCTCTGC |
| | | GCGGACGCAAGCGGAGATCTTGCGCCGCCTATGGAGGCTATATCAACTGCATGGCC |
| | | GTGCTGATGGAGAGCAACGCCGATCCTAATATCCAAGACAAGGAGGGACGCACAG |
| | | CCCTGCACAGGGGCAGAAAGAGAAGGTCTGATAACCGGCTGCCCCCCAAGAAGGT |
| | | GCCCGGATTTTGCAGCTTCCGCGACGGCCTGAGCTTCCTGGTGCACTGCTGTAACGT |
| | | GATCATCACAGCCCAGAGAGGCCGGAAGAGGAGATCCCCATACTCTACCGCCGTGT |
| | | CTACCTCTGGCTGCGGAGAGCACCTGGTGCACACCATCCTGGCAAGAGAGTGTTCC |
| | | CACGCCCTGCAGGCAGAGGACGCCAGAGGCCGCAAGAGAAGGTCCGGCGTGCTGA |
| | | AACAGTATCAGATCAAGGGCCTGGAGTGGCTGGTGTCCTGTACCACAACAACCCGG |
| | | GGCAGAAAGAGGAGGTCCTCTAAGTCTCCCTCCAAGAGAAGCAAGTCCCAGGACC |
| | | AGGCAAAGAAGAGCAAGAGCCCAACACTGAGAAGAAGGAGCCAGGAGAAGATCG |
| | | GCAAGGCAAGGGGAAGGAAGCGGAGGAGCTTTTCTCTGTGGAAGGGCTTTACACC |
| | | CTACTACGCCTGCCTGGGACCTCACACCGTGCTGACCTTCATCTTTCTGGAGCAGAT |
| | | GAACAAGGCATATAAGAGAGGCCGCAAGAGGCGCAGCGATTCTTTCCTGGTGGAG |
| | | CGGGGAAAGCTGCTGCACAAGCGGGATAACGACAAAGTGGATACCCAGGAGGAGA |
| | | ATTTCCTGCCAAAGTACCAGCGGGTGAGGGGCAGGAAAAGAAGGAGCTTTCGGAC |
| | | CTACCACCAGATGGTGCTGCACTCTAGGGTGCACCGCAGAGCCCGGAGAGAGATG |
| | | GACTCTGACGGAGACAGGGCCGCCAGAGCCCGCTGTAGAGGCAGGAAGCGCAGGA |
| | | GCCTGGAGGAGAATATCAATTCTGCCCTGGACATCAAGGTGCTGACCCAGAGCTCC |
| | | CAGTCTGCCGTGAGGGGAGGCCCCGGGAGATCCACCATGCCCACCAGAGGGCGGA |
| | | AGAGACGGTCCGGCGCCAGCGTGAACGCCCCCCCAGACCCTTGCAAGCAGAGCCC |
| | | AGTGAGACTGGCAGCCGGCTCTGGCCTGGCCTGCTTCCTGCTGTGGCAGCTGCAG |
| 41 | Patient | ATGGATTGGACATGGATTCTCTTTCTCGTTGCTGCAGCTACTCGCGTTCATAGCCAG |
| | PT3 | TCCTATCTGGACAGCGGCATCCACTCCGGCGCAACAACCACAGCACCTCCACTGTC |
| | | TGGAAAGGGAAATCCTGAGGAGGAGGATGTGGATACATCCCGCGGCAGGAAGAGA |
| | | CGGTCTTGGATGGGCGACTTTGTGACAGCCTGGATGGTGACAGATATGATGCTGCA |
| | | GGCAAAGCCCTACCCAGACTGGGGGAAGTCTGCCAGGGCCTTCTGGAAAAGGGGC |
| | | AGGAAGAGACGCTCCCCCACCGGCAGACCATACAATGTGGATACCGAGTCTGCAC |
| | | AGCTGTACCAGGGCCCCCACAATACCCTGTTTGGCTACTCCGTGGTGCTGCACTCCC |
| | | ACCGGGGCAGAAAGAGGCGCAGCGAGAAGCTGGCAGCCGAGGAGCAGTTTCAGGC |
| | | CCTGGTGAAGCAGATGTATCAGACACTGCAGGACAAGACCAACCAGATCGACCTG |
| | | CTGCAAGCCGAGAGGGGCCGCAAGAGAAGATCCAACGAGAAGATGGATTCCATCG |
| | | TGCAGTGCCGCGCACTGAACATCGCAGTGGACCTGGGCCAGATCCAGTACCTGTTT |
| | | TCTGACAAGACAGGCACCCTGAGGGGCAGAAAGCGGCGCAGCGAGGAGCTGCCCG |
| | | TGGTGGGACAGCTGCTGAGACTGCTGCTGCAGCACACCCCTCTGAGAACACACATG |
| | | CTGACAAACGCCATCCTGGTGCAGCAGATCAGGGGGAGGAAGAGGAGGTCTAACC |
| | | TGCCCCTGCTGCAGATCGAGCTGCTGCGGTCTGCAATCAAGGCCCTGAGGCCTGGC |
| | | CGCATCCTGGTGTACAGCACATGCACCCTGAGCAAGGCAAGAGGCCGGAAGCGGA |
| | | GAAGCGGATTTCAGTGTGCCGCCCCACTGCTGATCCTGACCAACAAATTCTGCTTCC |
| | | TGAGCACCTACGTCTTTGTGAGCACCTTCCTGACCGGCAAGGAGCTGAGAGGCAGG |
| | | AAACGCCGCTCCGTGACCGCAGCCTTCCTGGTGAGCTTTATGCCATACCACATCCA |
| | | GCGCAATATCCACCTGCACTTTCTGCACAATGAGACAAAGCCATGTGATTCTGTGA |
| | | GGGGACGGAAGAGAAGGTCTGCCAGGCCCGAGCTGCTGCAGAGCCACTTCATCCC |
| | | TACCATCGGCCGGCTGCGGAAGAAGGCCGGCAAGGTGGTGAGCGAGGAGGAGCAG |
| | | CTGAGGCTGAGAGGCAGAAAGCGCAGATCTGACCGGGTGGATGCCCAGGACGAAA |
| | | CCGCCGCCGAGTTCACCCTGCGGGTGAACCTGACCGCCGCCAGACTGGGCCAGCTG |
| | | GAGGGACTGCTGCAGGCCAGGGGCCGCAAGCGCCGGTCCCTGGTGCAGTCTAGAC |
| | | AGAACCTGGTGGCCGAGGAGCGCCTGTCCAGCGGCATCCTGGGCACAATCGGATTC |
| | | GGACGCAAGTCCCCACTGTCCAACAGAAGAGGCAGGAAGAGAAGGAGCATGGCCT |
| | | TTAGAACCATCTGCGTGCTGCTGGAGTATCTGTTCGTGCTGTCCGTGAGGGGCAGG |
| | | AAACGGAGATCCTTTGAGAAAGGACGCATCTATACATTTATCGGAGAGGTGGTGGT |
| | | GAGCGTGAACCCATATCAGCTGCTGAACATCTACGGCAGGGATACAATCGAGCAG |
| | | AGAGGAAGGAAGCGGAGAAGCTGGCCCGCCGTGTTCGCAAGAGTGAGCGTGTTCG |
| | | TGGATTGGATACACAAGGTGATGAGGCTGGGCCGGGGACGGAAGAGGAGGTCTGT |
| | | GTGCGTGAGCTCCGACACCCTGGATGGAAATTCCAGCAGCGCCGGCTACTCTTGCA |
| | | ACAGCCCCGCAAAGGTGTCCACCACCACAGATTCTCCAGTCAGAGGCAGGAAGAG |
| | | GAGAAGCGTGGAGGACGCCGTGGTGTTCTCTACAGAGCTGGAGGCCAGCGCCATCT |
| | | CCCCAAGGGTGGTGGTGCCTCTGGCCGAGACACACTGTGAGGAGCAGGGCAGAGG |
| | | CAGAAAGCGGAGATCTGCCGCCCAGCCCTACTCTAATCTGTCCAATCTGGACGTGC |
| | | TGAATCAGGTGGTGAGGGAGAGAGACACAAAGCTGCCAAAGCCTCAGCTGGAGCA |
| | | GCCTCGGGGCAGAAAGAGAAGAAGCGAGGTGAAGAGAAGCCAGCGCCTGCGCCG |
| | | CGGACCTTCCTCCCCTAGGCGCCCATATCAGGAGATGGAGTATGAGAGGAGAGGA |
| | | GGAAGAGGCGACAGAAGAGGCAGAAAGAGAAGGAGCGCATCTAGCCTGAATGAG |
| | | AACGTGGACCACAGCGCACTGCTGAAGCTGGATACCAGCGTGTCTGAGAGGGTGTC |
| | | TAGGTTCGATTCCAAGCCTGCCCCTAGAGGGCGGAAGCGGAGGTCTTGTCACCCAG |
| | | GCCCAAGGGACCTGCTGCTGCTGCACCTGCTGCAGAGAAAGGACAATATCGACCGC |
| | | GGCCGCAAGCGGAGAAGCGACCTGCTGCGCAGCGTCCTGCAGCAGAGACTGATCG |
| | | CCCTGGGCGGCATCATCGCCGCACGCATCAGCGTGAGAGGCAGAAAACGCCGGTC |
| | | CTATACCATGCAGAAACACCTGGAGATGAACCCCCACTTCGGCTCCCACCAGTATC |
| | | AGATCCTGCCCCTGCACTCTCAGATCCCAAGGGAGGAGCAGCGGAGAGGCCGCAA |
| | | GCGGAGGTCCTGTTCTTCTTCTCTGATGGCCCTGCAGAATGCCTACCAGGCAGTGCA |
| | | CAGCGGCCAGTGCCCCGCCGCCATCGTGGGCGGCATCAACGTGCTGCTGAAGAGG |
| | | GGGAGGAAGCGGAGGAGCTTCAGGCTGAACATCCAGGGCCTGGAGCGGACCCTGC |
| | | TGGGCTTCGTGTCTAAGACCCTGGACTCCGCATCCGCCCAGTTTGCAGCATCCGCCC |
| | | TGGTGAGGGGAAGGAAGAGGCGGAGCATCAATCCTCAGGGACTGTCCGATCCCTC |
| | | CCCAATGAGCGATCCAGTGCTGACCCAGGATATCAGCCCTCCTGCCCAGGGAGTGG |
| | | ACCACCGGCAGGTGAGAGGCAGGAAGCGCCGCTCCCTGGGCCCACACGAGGAGGA |
| | | TCAGGTGGTGTGCGGATTCAAGAAGCTGTCTGAGTGGGGCCTGTGTTTTCACCCCTC |
| | | CACAGATACCCACAAGAAGCTG |
| 42 | Patient | ATGGATTGGACTTGGATTCTTTTTCTCGTTGCTGCTGCTACTCGCGTTCACAGTCAG |
| | PT5 | GAGAAGGCCTTCTTCGCACAGCTGCAGCTGGGAGAGGAAACCGGAGAGTTCCTGC |
| | | CTATCCAGCCCGCCCAGCACATCCAGTCTGAGACCTCCGGAAGGGGCAGAAAAAG |
| | | GCGGAGCTCTTATCTGGATTCCGGAATTCACTCCGGCGCCACCACCACAGCACCCT |
| | | TCCTGTCTGGAAAAGGAAATCCTGAGGAGGAGGACGTGGACACAAGCCAGCGCGG |
| | | AAGGAAGCGGCGCTCCGGATGTTGCCCACCCGGCCACGGCTCTAGGAGCAGGCCC |
| | | CCAGCCCAGCCCGCCTGCTCTAGAGCAGAGCAGGATATCCTGTTTCTGCTGAGAAG |
| | | CCCAAGGGGCAGAAAGAGGAGGTCCCTGGTGTCCCTGCAGCCAGCCCAGATGCAC |
| | | AGGTTCTGGAGAGGGCTGTCCCTGCACCTGGACTCCGTGGATGCCGTGTATGAGAG |
| | | AACATCTGACCACAGAGGCAGGAAAAGGAGAAGCATCCAGCTGCCAATCATCCAG |
| | | CTGAGAAAAGTGTGGGCCGAGGCCGTGAGATACGTGTCTGGCCTGAAGGAGGATT |
| | | ACTCCAGACTGTTTCAGGGCCAACGCGGAAGGAAGAGGAGATCTATCTTTACCGGC |
| | | CAGGCAAATGTGGTGAGCGAGTATCTGCTGGCCTACCTGTTCCTGTCCCTGGTGGC |
| | | ATTTTTCAGACTGATCGCCTTTCTGAATCTGCGGGGCAGGAAGAGACGGTCTGGCA |
| | | GGAGATGTATCCGGGCCACCCCCAGAGCCTGCAGAGCAAGATGTGCCACATGCTGC |
| | | GTGCGGTCCAGCGCCGCCACAAGCCCCTGTTGCGGATGCAGAGGCCGCAAGAGGA |
| | | GATCCATGGCCTTCGCCCTGGCCATCAGAGACACCGCAACCTATGCCAGACAGCTG |
| | | ATGTTTTCTACCACCCTGCTGATCGTGTTCTTCACCGTCTGGGTGTTTCGCGGACGG |
| | | AAGCGCAGGTCTTGGCGGCCCCTGCTGAGAGGCCCCAGGCTGAGCCTGCACACCGC |
| | | CGCCAAGGCCGCCGCCACCGCCACAGAGACAACATGCCAGGATGTGGCAGCAACC |
| | | CGGGGCCGCAAGCGCCGCAGCGAGGCCCTGGCCGAGAACCCAGGCCTGGTGAACA |
| | | AGAGCTGTTATGAGGTGGGCTGGCTGATCAAGATGACACTGTCTAATCCTAGCGAG |
| | | CTGGACGAGCGCGGCCGCAAGAGACGCTCCCCTTCCGGCTCCTTCAGCACCCCAGG |
| | | CTCTGCAACATACGGCAGGTATAAGCCCAGCCCTGAGCGCTACACCGCAGCCCCAC |
| | | ACCCACTGCTGTCCCTGAGGGGCAGAAAGCGCAGGAGCCTGCAGACAAGACTGCA |
| | | GCCTGAGGAGAGCACAGAGACCCTGGACTCCAACTACGTGGTGGGCCACGTGCTG |
| | | AATAGCCGGAAGCAGAAGCAGCTGCTGCGGGGCAGAAAAAGAAGGAGCCTGGAG |
| | | GTGTCTCCCTTTTTCAAGTGTCACGAGGTGATCGGATCTTCTGTGCTGTTCATCCAC |
| | | GATAAGAAGGAGCAGGCAAAGGTGTGGATGATCGACAGAGGAAGGAAGCGGCGG |
| | | AGCGTGGCACAGCCAAAGAAGAAGCTGGGCAGGAGACTGGTGTTTCACAGCAACC |
| | | CCCCCAGCGGAAATAAGCAGCAGCTGCCCCTGGTGAATGAGACAAAGAGGGGCCG |
| | | GAAGAGGAGGTCTGTGCACATCAGGCGCAAGCACAGCCACCAGTACAGCTATCTG |
| | | TGCGAGGTGTGTAAGTATTATACAGTGACAAAGGGAGACATGGAGAGACACTGCG |
| | | CCAGAGGACGGAAGCGCCGGTCTTGTCACGTCGGCTCTGAGGAGACAAGGGCAAG |
| | | AGTGCCAGTGCACGACCTGCACGTGGGCATCACCAAGAGACTGAAAACAATGGAG |
| | | GTGCTGGAGGGCAGAGGCAGAAAGAGAAGGTCTGTGACCGACTACACCTTTGAGG |
| | | ATTGTCAGCTGGCACTGGCCGAGGGACACCTGCGCCTGCCTGCCGACACATGTCTG |
| | | CTGGAGTTCGCAAGACTGGTGAGGGGCCGGAAGCGCCGGAGCGAGATCCACCTGG |
| | | AGATCTTTGAGAGCGCCAGCGCCGGCACCCGCCTGCTGCTGGACCCCGCCACCGAC |
| | | CCAGATATCAACATCAACTCTATCAAGGATCGGGGAAGGAAGCGGAGATCCGTGT |
| | | ATGTGAGCACCCAGTTCCCTAAGTACATCCAGGATATCGTGGCAGCCACACTGAAG |
| | | CTGCCTGCCAACAAGGTGATGTGTCACGTGAGACGGGTGAGGGGGAGAAAGAGAA |
| | | GGAGCCTGACCCAGAGCAAGAGCTCCCCAAAGAGGGGCTTCTTTAGGGAGGAGAC |
| | | AGACAGGCTGATCAAAAACCTGCTGGGCAAGAGGATCTCCAAGCTGATCAGAGGC |
| | | CGGAAGAGGCGCAGCAACAACATGATCCACCAGGTGCCAATCAAGTCCCTGCCTC |
| | | AGGAGTGGCTGTGGTGCGAGATGTGGTGCGACGACGCATCCAAGAAGCGGGCAAA |
| | | GACAAGAGGACGCAAGCGCCGCTCTCCTGAGAATATCGAGATCACCGTGACCCTGT |
| | | TTAAAGATCCTCACGCCGAGGAGTTCGAGGGCAAGGAGTGGACATTTGTGATCGAG |
| | | AATGAGTCCCCAAGGGGAAGGAAGAGACGGTCCGCCGCCTCTAGACCACGGCTGG |
| | | ACCCTTGGAAGCTGGTGTCCTTTTCTCGCACCCTGACCATCAGCCCCCCTAGCCGCC |
| | | CAGATACCCCTGAGTCCCCTCGCGGGAGAAAGAGAAGATCCTCCAGCAAGACAAC |
| | | ATCCGTGCTGCAGTGGGCCGAGAAGGGCTATTACACCCGCTCTAACAATCTGGTGA |
| | | CACTGGAGAATGGCAAGCAGCTGACCGTGCGGGGCAGGAAGCGCAGAAGCCTGGA |
| | | CTGGCAGCCTAGGGAGAATCCATTTCAGAATCTGAAGGTGCTGTCCGTGAGCGATC |
| | | AGCAGCAGAATTTTCTGGAGCTGTGGAGCGAGATCCTGAGAGGCAGAAAGCGGCG |
| | | CAGCGAGAGATGGGAGTGCACCACCTACTGGTATATCTTCGTGTTCTGCTCCGCCC |
| | | ACCCTGCAGTGACCGAGATGGCCCTGTTCGTGACAGTGTTCGGCCTGCGCGGGAGG |
| | | AAGAGGCGGTCCTTTTACAAGAAGTGCGCACAGCTGACAGTGAATCTGACAAGATT |
| | | CCCAGAGACCGTGCCTGGCGAGCTGGTGGTGCCCGTGGCAGGCTCCTGCGTGGTGA |
| | | GAGGCCGCAAGAGAAGGTCTAACAATGTGGACCTGATCCTGGCCACCCCACCATTT |
| | | TCTAGACTGGAGAAACTGTACTCCGGCGAGGTGGTGAGAGGCAGGAAGCGCAGGT |
| | | CCAAGACCATGAAGGATTTTGTGCGGAGGAGGTGCTGGGCAAGGAAGTATAAGCT |
| | | GGTGACCTCTGGACCTTGGCTGGAGGTGCCCCCAATCGCACTGAGAAGGGGCAGA |
| | | AAGCGGAGGAGCGCCAGGAGAATGGCCTTTCTGGCCAAGAAGGGCTACAGGCACG |
| | | ATTCTTCCACAGCAGTGGTGGGGAGCCCCAGGGGCCACGGCCAGTCTAGAGAGAC |
| | | CCGCGGCAGGAAGCGGAGATCTTGGCGGCGGGCACAGAAGCTGCATTTCCTGCAG |
| | | AGGGGCGGGCTGAAGCAGCTGAGCGCCCGGGTGAGGCCAGAGCTGAAAACCAAGC |
| | | AGCAGATCCTGAGGGGCCGCAAACGGAGGAGCTCTGGCCGGCTGGATCAGCTGCT |
| | | GTCTCTGTCCATGGTGTGGGCCAACCACGTGTTCCTGGGATGTAGCTACAATAAGG |
| | | ACCTGCTGGATAAGGTGATGAGGGGCAGGAAGAGGCGCAGCGAGCTGGGCTCCCT |
| | | GATCCGCCAGCTGTCCCTGTGCTCTGTGAAGCTGTACGCCGATCCATCTGTCCCAGA |
| | | TGTGGTGATCGACATCCTGCAGCAGATCAGAGGCAGGAAGAGAAGAAGCCCTGAG |
| | | CTGTCTATCAGCAGCCTGACATCTCCTATCCCAGAGAACAGCACAGAAACCGAGGT |
| | | GGCCCTGTTCAGGATCAGAGACAGAGATTCCGGCGAGCGCGGCCGGAAGCGCAGA |
| | | AGCACCTCCGAGCTGCTGGGCGAGGGCGCCTACGCCAAGGTGCAGGGCGCAATGT |
| | | CCCTGCAGAACGGAAAGGAGTACGCAGTGAAGATCATCGAGAAGCAGCGCGGCAG |
| | | GAAGAGACGCTCTGATTGGGAGGGCAACAGGGCATATAGCCAGTACGACAAGTTC |
| | | CACATCGGAAACGAGAAGCAGAACTATAGACTGTACCTGAAGGGCCACACAGGCA |
| | | CCCGGGGCAGAAAGAGGCGGAGCCTGCGGCCAAAGCTGATCGAGGAGCTGGCCGC |
| | | AACCCGGATCGTGGACGTGTCCACCGGAGATTCCCACTGTCTGGCCCTGTCCCACG |
| | | ATAACGAGGTGAGAGGACGGAAGAGAAGGTCTTATGCCTACGACAAAAGAGCCAA |
| | | CCCACAGGTGGGAGCCGCCTTCCCCCACATCAAGCACAACTACGAGTGTCTGGTGT |
| | | ATGTGCAGCTGCCATTCATGCGCGGCAGAAAGAGACGCAGCAGGCGCTATCCTTCT |
| | | ATGGCCCGCATCCACAGCATGACCATCGAGGCCCCCATCGCAAAGGACGGACTGC |
| | | GGCGCCTGAGCGGCAATGAGTACGTGTTTAGGGGCAGAAAGAGACGGTCCATCTC |
| | | CACAGCAGTGAGGCCCACCACACCCACAACCTGCAGCAGGAGGTGGTGGCGCCCA |
| | | TCCAGCGTGGGAGGCAGCGCCTCTGGCTCCAGGCCTCCA |

TABLE 7
Amino Acid Sequences

| SEQ ID | | |
|---|---|---|
| NO | Name | Amino Acid Insert Sequence |
| 43 | Patient | MDWTWILFLVAAATRVHSQSYLDSGIHSGATTTAPYLSGKGNPEEEDVDTSRGRKRRS |
| | PT4 | HLVSRKSISAALEHKVSALLPPAEQTGTWKLKTRGRKRRSDLRSRIEVLKRKVIEKLQHI |
| | | QLLQKNVRAQLVDRGRKRRSSKDLLRQLPASNFNSLRFLIVHLKRVVDHAEENRGRKR |
| | | RSTLLLDGNPFRVPQAAILMKGTAAILEYLRDRIPRGRKRRSSILESSSAFPDNAARREV |
| | | ESPPAVCPSDGCTWKRGRKRRSGQPYFEAFKKKMPIAFVAKGINKLLNKLFLINERGRK |
| | | RRSMEDLQDMFIVHTIEEIEGLIAAHDQFKSTLPDARGRKRRSMGGQTALNCGEELFKR |
| | | GVLKEYGVKVLGTSVESRGRKRRSAKGVNGSSQAPTTGKYCRLCDIQFNNLSNFITHR |
| | | GRKRRSHMKLKVQGMECLDGCYVHSSIVPTFHRRCTWLLRGRKRRSQASRGEVPSGA |
| | | YGYSYIPSGAYVYPPPVANGMYRGRKRRSYSSLRSIVVANYEESIKMPISEPAPGKKKS |
| | | QIQRGRKRRSDLQVSAKIGFLKHHENKTYLAVGSMKTLMLNVSRGRKRRSELILPSGA |
| | | RVGHRSLRRYYKQRFGLSRAVAVAKRGRKRRSMLGEQGFQSLMETVDTGIVTQLQEF |
| | | LQGFRGRKRRSRQIWWERLQGCQHIVEDWQKILMVRSLVVSPHERGRKRRSSTTGSNY |
| | | YVRILSTIDGELLKPNASVALHKHSNRGRKRRSANELCEVNRKGCPSADPCLPYFCVQG |
| | | CKLGEASRGRKRRSFEDCTSENTLIKYMTDRILLRESLREADLDHYSRGRKRRSWIRPL |
| | | ASGSYPSRKAGATLVVYKDLLVLFGGWTRGRKRRSSSDYSTSEMLVNVGNLPLDEFYP |
| | | AVSMVALMRIRGRKRRSCESPTAHCNVLNWEQVHRLDGILSETIPIHGRGRGRKRRSN |
| | | KQNKTPLDKSTSGVSEILLKTQMKMSLKCLAARGRKRRSYVCGGMFFFVSFEGKIVMH |
| | | KIIYMVLFLFCVALRGRKRRSLPTPGSWWEQLTQASRAYASGGTEGFPLSRWAPRGRK |
| | | RRSYSTLSLFDKYKGKSVDTIRSSVIPRHGLQSLGKRGRKRRSMNISLVWCLLVLSFAIR |
| | | RWWRGRKRRSRVELQQEVEKLAHENSSMRLELDALRSKYEALQRGRKRRSSPQREPR |
| | | GGQLRTPRMWPSCSRSLESLRVGAKPRGRKRRSFTLWNESTHYQILLTSFPHVENHSCF |
| | | EHMHHIPRGRKRRSDFVVEAIGTEVGTLGFSIERPSQAKIECDDKGDRGRKRRSNVIDS |
| | | MLFVFGYRAQKNKIRGRKRRSMMGSARVAELLLLHGAEPNCTDPATLTRPVHDARGR |
| | | KRRSPPGLDKRLLPETLGPCYSNSQPVWLCLTPRQPLRGRKRRSSFLLSPNNGNLEATS |
| | | NIEYAHVPHLSPAVIPTRRGRKRRSEEDEEDTYYTKDLPIHTCSYCGIHDPACVVYCNR |
| | | GRKRRSLISRRDAEVVLTSRELGSLALNQSTGLPTLTLPRGRKRRSDFSSNDSSTCTMGL |
| | | VLTLWSDTLIHLDGDGGFSRGRKRRSSSAAPSVMDLSSRASAALHPGIPSPNCMQSPIR |
| 44 | Patient | MDWTWILFLVAAATRVHSPDRKAAVSHWQQQSYLGSGIHSGATTTAPSLSGRGRKRR |
| | PT1 | SGQSPTLLTSACFFPSAAFTAEFLFSLGSLCQHLRGRKRRSRLASAPPQKQKQKLGERLF |
| | | PLIQAMHPTLAGKIRGRKRRSCGHLYAYDWISIPVVYTQVVTVAVYSFFLTCLVRGRK |
| | | RRSDCMSEDGYKLDRIKGEFEFHNVTFHYPSRPEVKRGRKRRSMANDSPAKSLVDIDL |
| | | PSLRDPAGIFELVEVVGNRGRKRRSWLGIRISESPEPGQRTFTTFKFCQLPFYKYSEYRG |
| | | RKRRSPPLARPILPRERGAMDRIVEYLVGDGPQNRYALRGRKRRSTCVSLDDGLYECSC |
| | | APAYSGKDCQKKDGPCVINRGRKRRSLHVGLCSGPCEMAEQRCCVDYAKRGTAGCK |
| | | KCKRGRKRRSSFESYLKTMPPYFLGPLKKAVRMMGAPNLIADSRGRKRRSPKFHSYVS |
| | | LPFGCTRAVVEYRLLQAAYLAKPGDRGRKRRSGEYVDTGKLIDKINLPNFLKVYLNHK |
| | | PPFGNTMRGRKRRSMCTNARRVRKRWLPKINSMLPEGVEMYRTVMGSRGRKRRSPD |
| | | GPLLPSPGLLRLPGGPRHAVSGVPEVYPPGPRGRKRRSVGTQATKYNSEVVDKMIEEFL |
| | | SSFEEKIENLTERGRKRRSTNLPIFKLKESCVRRRYSDFKWLKNELERDSKIRGRKRRSLI |
| | | RWCLALRPSDRPTFEEIQKHPWMQDVLLPQERGRKRRSKFECGWCSGERRCTLHQHC |
| | | TNPSSPWLDWSSHNRGRKRRSHDHTGFLTEYVATRWYSAPEIMLNSKGYTKSIDRGRK |
| | | RRSSSEATEVNPESLAKECLEKSLLLLKFLPTGISSRGRKRRSVKKDRSASHLDHKLEIR |
| | | QWRRSFLVSLFFCIPVRGRKRRSLFTFAVGVNICLGFTAYRIKRAEGWEEGPPTVLRGR |
| | | KRRSMSQAPGAQLSPPTVYHERQRLELCARGRKRRSMSSNSFPYIEQSGGGEATELGQE |
| | | ATRGRKRRSQPCRKMLPDRSRAARYRANQKLVEYIVKAKGAERGRKRRSPVFHGMLE |
| | | RAPAEPCYRAPMEKLFYLPHVCSYTRGRKRRSTYSCVAENILGTGQRGSGAELDVQYP |
| | | PKKVTTVRGRKRRSSPSATEDSLQPATDLLNRSELPQSQKAMQTKDARGRKRRSDLRK |
| | | VPGTDPAFEVFPLHDHQCVLRGRKRRSDALDFKKDKGAFYLVFIWTMTRGRKRRSVD |
| | | NYKQIKADLDQLRLPVEKSELWVEKSSNYENRGRKRRSNAPENGYMKCSSEGDNYGA |
| | | TCEFSCIGGYELQGRGRKRRSESGSSGKSSSSLVRGVHLSSSGPALLAGLVSLDRGRKR |
| | | RSWTEKRASYELEFAKSTTKIAEAGKVSIQQQSHMRGRKRRSKILARKKSKRSALENSE |
| | | EHSTKYSNSNNSAGSGRGRKRRSAAAAAALAAALLLLRREDPGLGAGPSMAETEALR |
| | | GRKRRSLPSFPPFGSMNSNAAGSVSTQANTVQSGQLGGQRGRKRRSSGVVHRLEVDED |
| | | FEEHNAAKVHLMVHNLVPPFLRGRKRRSLQIKQQELYKNFLRFQVEEKKQREEAERER |
| | | LRI |
| 45 | Patient | MDWTWILFLVAAATRVHSEQQSLSSDADTLSLTDSSVDPPIQDPRGRKRRSDQNTEDY |
| | PT6 | LFQVILEKQIRIPHSLSVKAASVLKSRGRKRRSLREDGREQEIRDGGSLRMIHFGLVRGR |
| | | KRRSLLQLWPVQRLTVKKSTDNLFREQKSSLNYLRQKRGRKRRSQWLRAVAVESIHRF |
| | | CMQPQLLRSFCQSYDMKQHRGRKRRSLEAIVQLWRIPSFVTELYVNYDCDYYCSNLFE |
| | | ERGRKRRSTPPGQSPGSAPIIINIHRAQLTNPEVKYNYTEDRGRKRRSPYATVAPSTLAH |
| | | PQAQVLARQQALQHAQTLAHARGRKRRSQRGLLQQIGDALSSQRGRVPPAAPPAQPR |
| | | VPVTRGRKRRSMFRSKRSGLVRRLWRSGVVPDREEGGSGGGGGGRGRKRRSLEIKVSP |
| | | PEGAETRKVREIGPLCGRRRPRVRKSRGRKRRSTPNFIIPAQRAEPMRIVRQPMPPPGDL |
| | | EPPFQPRGRKRRSGTGALGLLLLFLLGYGLRRRPQKAEEPAPQAGGRGRKRRSDSHVSE |
| | | VLQENYRLKNKLEGLISEKNELKMKSERGRKRRSVSAYQVQSPSWMQPQPCILQHPGA |
| | | VLTPSMEHTRGRKRRSGRFSIVKKCIHKATRKYVAVKFVSKKMKKKEQARGRKRRSGI |
| | | LRDPGSEIEDRQYRIDLQSINIGTAQWHQLKRGRKRRSDGTQVLKIYVKQTESHYILVS |
| | | WKVNSNVMTSNLRGRKRRSPPGYIPDELHQVARNGLFTSINSEGEFIPESMDRGRKRRS |
| | | PKFRGVKMIPPGIHFLHYSSMDKANPKEVGPRMRGRKRRSNVSSAEGWHVNVTLSIRP |
| | | STGTGVMLALVSGNNRGRKRRSCQRSFRTFQALKKHLEASHLELSEADIQQLYGGRGR |
| | | KRRSDSVDFRLRVSEPKAVFAKEQLACREVQAEVGASRGRKRRSDIKDGRFPYGSTQD |
| | | YLKPIILIKLVQLGMAKDDRGRKRRSENYRNLAFLGIALSKPDLITFLEQGKEPWNMKQ |
| | | RGRKRRSWNSLGKMSREEAMSAYVTEMKLVAQKVIDTVPLRGRKRRSTFQCVVSPSD |
| | | VAVVWFWDGALLQPSEKFAISQSRGRKRRSHVDLFDLESGEYLCPLCKSLYNTVIPIIPL |
| | | QPQRGRKRRSSEIISKLYIPKRKIISPRSIKDVLPPVEEAVDR |
| 46 | Patient | MDWTWILFLVAAATRVHSPDRKAAVSHWQQQSYLGSGIHSGATTTAPSLSGRGRKRR |
| | PT2 | SGDNTGEQVAVKSPKPESGGNHIADLKKEIEILRRGRKRRSKDHGVNSFLVYMAFKDL |
| | | FQLTDCQIYEVLSVIRRGRKRRSNTDGKIEFISTMEGYTYPVYGVQWHPEKAPYEWRG |
| | | RKRRSNNSVLQKQQLELMDTVDNLVNLCTKEGVLLKGGRGRKRRSDSNREKDMGLF |
| | | EVFSQHLPTTEPVDSSVSSSISRGRKRRSLKAEVFTEILNYIYSSTVIVKRQETVTDLAAA |
| | | GRGRKRRSHPNNVVSIKYCSHSGLMFSVSTSYIKVWDIRDSRGRKRRSTKNKRRKEYF |
| | | LAVQIRSLEEKIKVKSTEVEILERGRKRRSLVKPQKENEQAEKKNISLAFFLYDLLSLMD |
| | | RGFRGRKRRSVDIVILGLSYANLQTGSGVLSDSLSGGMQLLPDRGRKRRSITKITRRRHE |
| | | NLPHGVASVKEWFNYVTATRNEERGRKRRSAVLKVGWLFPLSEVPNFTLLMDGCGC |
| | | WRLKEDQRGRKRRSLVAALLLGSVCCGSAQLLENKTKSVEFTFRGRKRRSSHVFGTNT |
| | | SSLELFLMNGKIKGPCWLEVKSPQLRGRKRRSSPYDHTPGMAGSLGYHPYEAPLGSYP |
| | | YGDPAYRRGRKRRSMTGSSLSLAHLLIISGLLCYSARGRKRRSAEEGDELQQRCLDLER |
| | | HLMLLSEEKQSLAQENARGRKRRSTMYHGVPVVGIPLFGDHYETMTRVQAKGMGILL |
| | | RGRKRRSQPASHILNSEKFPFSKYEDCLEQQMPSLRESPMRGRKRRSVEKSPAKKKATK |
| | | KAVGAGAAKRKATGPPVSELIRGRKRRSLLAAGPSAAAAKPNIPKVLLPFTRATRVNFT |
| | | LERGRKRRSLAPQDMRVFYAHTARAQLPVWNNWTQRAARYECRGRKRRSPAPLRRP |
| | | LAVLHAPVPTTALARLSRPQRACPSSRGRKRRSKHWFVLTDSSLKYYRDSTAEGADEL |
| | | DGEIDLRSRGRKRRSVKFLRKPGGNLGKVYHPGSMLSLAPTKGLLNEPRGRKRRSDYQ |
| | | VYLNASKVPGFADDPTELSCRVVDTKSGEARGRKRRSQRDDPVLETSHQNFRRSRYQE |
| | | AASPREALIRLRRGRKRRSHLLNYVWPNVFETSPHVIEAVMGALEGLRVAIGRGRKRR |
| | | SSRTSMDDLKAFTSLSLYMKPLRGRKRRSCAAYGGYINCMAVLMESNADPNIQDKEG |
| | | RTALHRGRKRRSDNRLPPKKVPGFCSFRDGLSFLVHCCNVIITAQRGRKRRSPYSTAVS |
| | | TSGCGEHLVHTILARECSHALQAEDARGRKRRSGVLKQYQIKGLEWLVSCTTTTRGRK |
| | | RRSSKSPSKRSKSQDQAKKSKSPTLRRRSQEKIGKARGRKRRSFSLWKGFTPYYACLGP |
| | | HTVLTFIFLEQMNKAYKRGRKRRSDSFLVERGKLLHKRDNDKVDTQEENFLPKYQRV |
| | | RGRKRRSFRTYHQMVLHSRVHRRARREMDSDGDRAARARCRGRKRRSLEENINSALD |
| | | IKVLTQSSQSAVRGGPGRSTMPTRGRKRRSGASVNAPPDPCKQSPVRLAAGSGLACFLL |
| | | WQLQ |
| 47 | Patient | MDWTWILFLVAAATRVHSQSYLDSGIHSGATTTAPPLSGKGNPEEEDVDTSRGRKRRS |
| | PT3 | WMGDFVTAWMVTDMMLQAKPYPDWGKSARAFWKRGRKRRSPTGRPYNVDTESAQ |
| | | LYQGPHNTLFGYSVVLHSHRGRKRRSEKLAAEEQFQALVKQMYQTLQDKTNQIDLLQ |
| | | AERGRKRRSNEKMDSIVQCRALNIAVDLGQIQYLFSDKTGTLRGRKRRSEELPVVGQL |
| | | LRLLLQHTPLRTHMLTNAILVQQIRGRKRRSNLPLLQIELLRSAIKALRPGRILVYSTCTL |
| | | SKARGRKRRSGFQCAAPLLILTNKFCFLSTYVFVSTFLTGKELRGRKRRSVTAAFLVSF |
| | | MPYHIQRNIHLHFLHNETKPCDSVRGRKRRSARPELLQSHFIPTIGRLRKKAGKVVSEEE |
| | | QLRLRGRKRRSDRVDAQDETAAEFTLRVNLTAARLGQLEGLLQARGRKRRSLVQSRQ |
| | | NLVAEERLSSGILGTIGFGRKSPLSNRRGRKRRSMAFRTICVLLEYLFVLSVRGRKRRSF |
| | | EKGRIYTFIGEVVVSVNPYQLLNIYGRDTIEQRGRKRRSWPAVFARVSVFVDWIHKVM |
| | | RLGRGRKRRSVCVSSDTLDGNSSSAGYSCNSPAKVSTTTDSPVRGRKRRSVEDAVVFS |
| | | TELEASAISPRVVVPLAETHCEEQGRGRKRRSAAQPYSNLSNLDVLNQVVRERDTKLP |
| | | KPQLEQPRGRKRRSEVKRSQRLRRGPSSPRRPYQEMEYERRGGRGDRRGRKRRSASSL |
| | | NENVDHSALLKLDTSVSERVSRFDSKPAPRGRKRRSCHPGPRDLLLLHLLQRKDNIDRG |
| | | RKRRSDLLRSVLQQRLIALGGIIAARISVRGRKRRSYTMQKHLEMNPHFGSHQYQILPL |
| | | HSQIPREEQRRGRKRRSCSSSLMALQNAYQAVHSGQCPAAIVGGINVLLKRGRKRRSFR |
| | | LNIQGLERTLLGFVSKTLDSASAQFAASALVRGRKRRSINPQGLSDPSPMSDPVLTQDIS |
| | | PPAQGVDHRQVRGRKRRSLGPHEEDQVVCGFKKLSEWGLCFHPSTDTHKKL |
| 48 | Patient | MDWTWILFLVAAATRVHSQEKAFFAQLQLGEETGEFLPIQPAQHIQSETSGRGRKRRSS |
| | PT5 | YLDSGIHSGATTTAPFLSGKGNPEEEDVDTSQRGRKRRSGCCPPGHGSRSRPPAQPACS |
| | | RAEQDILFLLRSPRGRKRRSLVSLQPAQMHRFWRGLSLHLDSVDAVYERTSDHRGRKR |
| | | RSIQLPIIQLRKVWAEAVRYVSGLKEDYSRLFQGQRGRKRRSIFTGQANVVSEYLLAYL |
| | | FLSLVAFFRLIAFLNLRGRKRRSGRRCIRATPRACRARCATCCVRSSAATSPCCGCRGR |
| | | KRRSMAFALAIRDTATYARQLMFSTTLLIVFFTVWVFRGRKRRSWRPLLRGPRLSLHT |
| | | AAKAAATATETTCQDVAATRGRKRRSEALAENPGLVNKSCYEVGWLIKMTLSNPSEL |
| | | DERGRKRRSPSGSFSTPGSATYGRYKPSPERYTAAPHPLLSLRGRKRRSLQTRLQPEEST |
| | | ETLDSNYVVGHVLNSRKQKQLLRGRKRRSLEVSPFFKCHEVIGSSVLFIHDKKEQAKV |
| | | WMIDRGRKRRSVAQPKKKLGRRLVFHSNPPSGNKQQLPLVNETKRGRKRRSVHIRRK |
| | | HSHQYSYLCEVCKYYTVTKGDMERHCARGRKRRSCHVGSEETRARVPVHDLHVGITK |
| | | RLKTMEVLEGRGRKRRSVTDYTFEDCQLALAEGHLRLPADTCLLEFARLVRGRKRRSE |
| | | IHLEIFESASAGTRLLLDPATDPDININSIKDRGRKRRSVYVSTQFPKYIQDIVAATLKLP |
| | | ANKVMCHVRRVRGRKRRSLTQSKSSPKRGFFREETDRLIKNLLGKRISKLIRGRKRRSN |
| | | NMIHQVPIKSLPQEWLWCEMWCDDASKKRAKTRGRKRRSPENIEITVTLFKDPHAEEF |
| | | EGKEWTFVIENESPRGRKRRSAASRPRLDPWKLVSFSRTLTISPPSRPDTPESPRGRKRRS |
| | | SSKTTSVLQWAEKGYYTRSNNLVTLENGKQLTVRGRKRRSLDWQPRENPFQNLKVLS |
| | | VSDQQQNFLELWSEILRGRKRRSERWECTTYWYIFVFCSAHPAVTEMALFVTVFGLRG |
| | | RKRRSFYKKCAQLTVNLTRFPETVPGELVVPVAGSCVVRGRKRRSNNVDLILATPPFSR |
| | | LEKLYSGEVVRGRKRRSKTMKDFVRRRCWARKYKLVTSGPWLEVPPIALRRGRKRRS |
| | | ARRMAFLAKKGYRHDSSTAVVGSPRGHGQSRETRGRKRRSWRRAQKLHFLQRGGLK |
| | | QLSARVRPELKTKQQILRGRKRRSSGRLDQLLSLSMVWANHVFLGCSYNKDLLDKVM |
| | | RGRKRRSELGSLIRQLSLCSVKLYADPSVPDVVIDILQQIRGRKRRSPELSISSLTSPIPEN |
| | | STETEVALFRIRDRDSGERGRKRRSTSELLGEGAYAKVQGAMSLQNGKEYAVKIIEKQ |
| | | RGRKRRSDWEGNRAYSQYDKFHIGNEKQNYRLYLKGHTGTRGRKRRSLRPKLIEELA |
| | | ATRIVDVSTGDSHCLALSHDNEVRGRKRRSYAYDKRANPQVGAAFPHIKHNYECLVY |
| | | VQLPFMRGRKRRSRRYPSMARIHSMTIEAPIAKDGLRRLSGNEYVFRGRKRRSISTAVR |
| | | PTTPTTCSRRWWRPSSVGGSASGSRPP |

TABLE 4
Patient mutations sequences

| SEQ ID | | | SEQ ID | | |
|---|---|---|---|---|---|
| NO | Name | DNA Sequence | NO | Name | mRNA Sequence |
| 19 | Patient | CAGTCTTACCTGGATTCTGG | 20 | | CAGUCUUACCUGGAUUCUGG |
| | PT4 | CATCCACAGCGGAGCCACC | | | CAUCCACAGCGGAGCCACCAC |
| | CTNNB1 | ACCACCGCCCCTTATCTGAG | | | CACCGCCCCUUAUCUGAGCGG |
| | p.S45Y | CGGCAAGGGAAACCCAGAG | | | CAAGGGAAACCCAGAGGAGG |
| | | GAGGAGGATGTGGATACAT | | | AGGAUGUGGAUACAUCC |
| | | CC | | | |
| 21 | Patient | CCTGACAGGAAGGCCGCCG | 22 | | CCUGACAGGAAGGCCGCCGU |
| | PT1 | TGTCCCACTGGCAGCAGCA | | | GUCCCACUGGCAGCAGCAGA |
| | CTNNB1 | GAGCTATCTGGGCAGCGGC | | | GCUAUCUGGGCAGCGGCAUC |
| | D32G | ATCCACTCTGGCGCCACAAC | | | CACUCUGGCGCCACAACCACC |
| | | CACCGCACCATCTCTGTCTG | | | GCACCAUCUCUGUCUGGG |
| | | GG | | | |
| 23 | Patient | GAGCAGCAGTCTCTGTCTAG | 24 | | GAGCAGCAGUCUCUGUCUAG |
| | PT6 | CGACGCCGATACCCTGTCTC | | | CGACGCCGAUACCCUGUCUC |
| | AXIN1 | TGACCGACTCTAGCGTGGAC | | | UGACCGACUCUAGCGUGGAC |
| | p.G342fs | CCACCTATCCAGGACCCA | | | CCACCUAUCCAGGACCCA |
| 25 | Patient | CCTGATCGGAAAGCCGCAG | 26 | | CCUGAUCGGAAAGCCGCAGU |
| | PT2 | TGAGCCACTGGCAGCAGCA | | | GAGCCACUGGCAGCAGCAGU |
| | CTNNB1 | GTCCTACCTGGGCAGCGGA | | | CCUACCUGGGCAGCGGAAUC |
| | p.D32G | ATCCACTCTGGAGCCACAAC | | | CACUCUGGAGCCACAACAAC |
| | | AACCGCACCATCTCTGTCTG | | | CGCACCAUCUCUGUCUGGC |
| | | GC | | | |
| 27 | Patient | CAGTCCTATCTGGACAGCGG | 28 | | CAGUCCUAUCUGGACAGCGG |
| | PT3 | CATCCACTCCGGCGCAACAA | | | CAUCCACUCCGGCGCAACAAC |
| | CTNNB1 | CCACAGCACCTCCACTGTCT | | | CACAGCACCUCCACUGUCUGG |
| | p.S45P | GGAAAGGGAAATCCTGAGG | | | AAAGGGAAAUCCUGAGGAGG |
| | | AGGAGGATGTGGATACATC | | | AGGAUGUGGAUACAUCC |
| | | C | | | |
| 29 | Patient | TCTTATCTGGATTCCGGAAT | 30 | | UCUUAUCUGGAUUCCGGAAU |
| | PT5 | TCACTCCGGCGCCACCACCA | | | UCACUCCGGCGCCACCACCAC |
| | CTNNB1 | CAGCACCCTTCCTGTCTGGA | | | AGCACCCUUCCUGUCUGGAA |
| | p.S45F | AAAGGAAATCCTGAGGAGG | | | AAGGAAAUCCUGAGGAGGAG |
| | | AGGACGTGGACACAAGCCA | | | GACGUGGACACAAGCCAG |
| | | G | | | |

TABLE 5
Patient Amino Acid Mutation Sequences

| SEQ | | |
|---|---|---|
| ID NO | Name | Protein Sequence |
| 31 | Patient PT4 | QSYLDSGIHSGATTTAPYLSGKGNPEE |
| | CTNNB1 | EDVDTS |
| | p.S45Y | |
| 32 | Patient PT1 | PDRKAAVSHWQQQSYLGSGIHSGATTT |
| | CTNNB1 | APSLSG |
| | D32G | |
| 33 | Patient PT6 | EQQSLSSDADTLSLTDSSVDPPIQDP |
| | AXIN1 | |
| | p.G342fs | |
| 34 | Patient PT2 | PDRKAAVSHWQQQSYLGSGIHSGATTT |
| | CTNNB1 | APSLSG |
| | p.D32G | |
| 35 | Patient PT3 | QSYLDSGIHSGATTTAPPLSGKGNPEE |
| | CTNNB1 | EDVDTS |
| | p.S45P | |
| 36 | Patient PT5 | SYLDSGIHSGATTTAPFLSGKGNPEEE |
| | CTNNB1 | DVDTSQ |
| | p.S45F | |

TABLE 6
DNA Insert Sequences into pGX0001

| SEQ ID | | |
|---|---|---|
| NO | Name | DNA Insert Sequence |
| 37 | Patient | ATGGATTGGACATGGATTCTCTTTCTCGTTGCAGCAGCCACACGCGTTCACAGTCAG |
| | PT4 | TCTTACCTGGATTCTGGCATCCACAGCGGAGCCACCACCACCGCCCCTTATCTGAGC |
| | | GGCAAGGGAAACCCAGAGGAGGAGGATGTGGATACATCCAGAGGCCGCAAGCGG |
| | | AGATCTCACCTGGTGTCCAGAAAGTCCATCTCTGCCGCCCTGGAGCACAAGGTGAG |
| | | CGCCCTGCTGCCCCCCGCCGAGCAGACCGGAACATGGAAGCTGAAAACCAGAGGC |
| | | AGGAAGAGAAGATCCGACCTGCGGTCCCGCATCGAGGTGCTGAAGAGGAAGGTGA |
| | | TCGAGAAGCTGCAGCACATCCAGCTGCTGCAGAAGAATGTGCGGGCACAGCTGGT |
| | | GGACCGGGGAAGGAAGCGGCGCAGCAGCAAGGACCTGCTGCGGCAGCTGCCTGCC |
| | | TCCAACTTTAACTCTCTGCGCTTCCTGATCGTGCACCTGAAAAGAGTGGTGGATCAC |
| | | GCCGAGGAGAATCGGGGCAGAAAGCGCCGCTCCACCCTGCTGCTGGATGGCAACC |
| | | CTTTCAGAGTGCCCCAGGCCGCAATCCTGATGAAGGGCACCGCCGCAATCCTGGAG |
| | | TACCTGAGGGACAGAATCCCTAGGGGCAGGAAGCGGCGGTCCTCCATCCTGGAGA |
| | | GCAGCTCCGCCTTTCCCGATAATGCCGCCCGCAGGGAGGTGGAGAGCCCCCCAGCA |
| | | GTCTGTCCATCTGACGGCTGCACATGGAAGAGAGGACGCAAGCGGAGATCCGGGC |
| | | AGCCATACTTCGAGGCATTCAAGAAGAAGATGCCCATCGCATTCGTGGCAAAGGGC |
| | | ATCAATAAGCTGCTGAACAAGCTGTTTCTGATCAACGAGAGGGGCCGGAAGCGCA |
| | | GGAGCATGGAGGATCTGCAGGACATGTTTATCGTGCACACAATCGAGGAGATCGA |
| | | GGGCCTGATTGCAGCCCACGATCAGTTCAAGAGCACCCTGCCAGACGCCAGAGGCC |
| | | GGAAGAGGCGCAGCATGGGCGGCCAGACCGCCCTGAATTGCGGCGAGGAGCTGTT |
| | | TAAGAGAGGCGTGCTGAAGGAGTACGGCGTGAAGGTGCTGGGCACCTCTGTGGAG |
| | | AGCCGCGGCAGGAAGCGGCGCTCCGCCAAGGGCGTGAACGGCTCTAGCCAGGCCC |
| | | CTACCACCGGAAAGTACTGTCGCCTGTGCGACATCCAGTTTAATAATCTGAGCAAC |
| | | TTTATCACCCACAGAGGACGGAAGCGGCGGAGCCACATGAAGCTGAAAGTGCAGG |
| | | GAATGGAGTGTCTGGATGGCTGTTATGTCCACAGCAGCATCGTGCCAACATTCCAT |
| | | AGAAGATGCACATGGCTGCTGCGCGGCAGAAAAAGGAGATCTCAGGCAAGCAGAG |
| | | GCGAGGTGCCAAGCGGCGCCTACGGCTACAGCTATATACCTTCCGGAGCCTATGTG |
| | | TACCCCCCACCTGTGGCCAACGGCATGTATAGGGGCAGGAAGAGAAGGAGCTATT |
| | | CCAGCCTGAGATCTATCGTGGTGGCCAATTACGAGGAGTCCATCAAGATGCCAATC |
| | | AGCGAGCCAGCCCCTGGCAAGAAGAAGTCCCAGATCCAGCGCGGCAGGAAGAGGC |
| | | GCTCTGACCTGCAGGTGAGCGCCAAGATCGGCTTCCTGAAGCACCACGAGAATAAG |
| | | ACATACCTGGCCGTGGGCTCTATGAAAACCCTGATGCTGAACGTGAGCAGGGGCAG |
| | | AAAGCGCAGGTCCGAGCTGATCCTGCCTAGCGGCGCCAGGGTGGGCCACAGGAGC |
| | | CTGAGGAGATACTATAAGCAGAGGTTCGGCCTGTCTAGAGCCGTGGCCGTGGCCAA |
| | | GCGGGGACGCAAGAGGAGGTCTATGCTGGGCGAGCAGGGATTCCAGTCCCTGATG |
| | | GAAACCGTGGATACAGGCATCGTGACACAGCTGCAGGAGTTTCTGCAGGGATTCAG |
| | | GGGCCGCAAGAGGAGGTCCCGCCAGATCTGGTGGGAGAGACTGCAGGGCTGCCAG |
| | | CACATCGTGGAGGATTGGCAGAAGATCCTGATGGTGCGCTCCCTGGTGGTGAGCCC |
| | | CCACGAGAGAGGACGGAAGCGCAGGTCCAGCACCACCGGATCTAACTACTACGTG |
| | | AGAATCCTGTCTACAATCGATGGCGAGCTGCTGAAGCCAAATGCCAGCGTGGCCCT |
| | | GCACAAGCACTCCAACAGGGGCCGCAAGCGCAGGAGCGCCAACGAGCTGTGCGAG |
| | | GTGAACAGAAAGGGCTGCCCAAGCGCCGATCCATGTCTGCCTTACTTCTGCGTGCA |
| | | GGGCTGTAAGCTGGGCGAGGCCTCCCGGGGACGCAAGCGGAGGTCTTTTGAGGATT |
| | | GCACATCTGAGAACACCCTGATCAAGTATATGACCGATCGCATCCTGCTGAGGGAG |
| | | TCTCTGCGCGAGGCAGACCTGGACCACTATAGCCGGGGAAGGAAAAGGAGAAGCT |
| | | GGATCAGACCACTGGCATCCGGCAGCTACCCTAGCCGCAAGGCAGGAGCCACACT |
| | | GGTGGTGTACAAGGATCTGCTGGTGCTGTTCGGGGGCTGGACACGCGGACGCAAGA |
| | | GAAGGAGCTCCTCCGACTACTCCACATCCGAGATGCTGGTGAACGTGGGAAACCTG |
| | | CCCCTGGACGAGTTTTATCCCGCCGTGAGCATGGTGGCCCTGATGCGCATCCGGGG |
| | | CCGCAAGAGAAGATCCTGCGAGAGCCCCACAGCACACTGTAATGTGCTGAATTGG |
| | | GAGCAGGTGCACAGACTGGACGGCATCCTGTCTGAGACAATCCCAATCCACGGAA |
| | | GGGGAAGGGGGAGGAAGAGAAGGTCCAATAAGCAGAATAAGACCCCACTGGATA |
| | | AGTCTACATCCGGGGTGTCTGAGATCCTGCTGAAAACCCAGATGAAAATGAGCCTG |
| | | AAGTGCCTGGCCGCCCGCGGACGGAAGAGAAGGTCTTACGTGTGCGGCGGCATGTT |
| | | CTTCTTTGTGAGCTTCGAGGGGAAGATCGTGATGCACAAGATCATCTATATGGTGCT |
| | | GTTCCTGTTTTGTGTGGCACTGAGAGGCAGAAAGAGGAGGTCCCTGCCTACCCCAG |
| | | GGTCTTGGTGGGAGCAGCTGACCCAGGCATCTCGGGCCTACGCAAGCGGAGGCAC |
| | | CGAGGGCTTTCCTCTGAGCCGGTGGGCCCCCAGGGGCAGAAAGAGAAGGTCTTATT |
| | | CCACCCTGTCCCTGTTTGACAAGTACAAGGGCAAGTCCGTGGATACCATCCGGAGC |
| | | TCCGTGATCCCCCGCCACGGCCTGCAGTCCCTGGGCAAAAGAGGCAGAAAGAGAA |
| | | GAAGCATGAACATCTCTCTGGTGTGGTGCCTGCTGGTGCTGTCCTTCGCCATCAGGA |
| | | GGTGGTGGCGCGGCAGGAAGCGCCGGAGCAGGGTGGAGCTGCAGCAGGAGGTGG |
| | | AGAAGCTGGCACACGAGAATAGCTCCATGCGGCTGGAGCTGGATGCCCTGAGGTC |
| | | CAAATATGAGGCACTGCAGAGGGGGAGAAAGAGAAGGTCCAGCCCTCAGAGAGA |
| | | GCCAAGAGGCGGCCAGCTGAGAACCCCAAGAATGTGGCCTTCTTGTTCCAGAAGCC |
| | | TGGAGTCTCTGAGAGTGGGAGCCAAGCCCCGGGGAAGGAAGAGACGCTCCTTCAC |
| | | ACTGTGGAACGAGAGCACCCACTATCAGATCCTGCTGACCTCTTTTCCCCACGTGG |
| | | AGAATCACTCTTGTTTTGAGCACATGCACCACATCCCAAGAGGCAGAAAGCGGCGG |
| | | TCTGATTTTGTGGTGGAGGCCATCGGAACCGAGGTGGGCACCCTGGGATTCTCCAT |
| | | CGAAAGACCTTCCCAGGCCAAGATCGAGTGCGACGACAAGGGCGATCGGGGCAGG |
| | | AAGCGCAGATCCAACGTGATCGACAGCATGCTGTTCGTGTTCGGATATAGAGCCCA |
| | | GAAGAATAAGATCCGCGGAAGGAAGAGGCGGTCTATGATGGGCAGCGCAAGGGTG |
| | | GCAGAGCTGCTGCTGCTGCACGGAGCAGAGCCCAACTGTACCGACCCTGCAACACT |
| | | GACCAGACCAGTGCACGACGCAAGAGGAAGGAAGAGAAGAAGCCCCCCAGGCCT |
| | | GGACAAGAGACTGCTGCCAGAGACACTGGGCCCCTGCTACTCCAATTCTCAGCCTG |
| | | TGTGGCTGTGTCTGACACCACGGCAGCCACTGAGAGGCAGGAAAAGAAGATCTTCC |
| | | TTCCTGCTGAGCCCTAATAATGGCAACCTGGAGGCCACATCTAATATCGAGTACGC |
| | | ACACGTGCCCCACCTGTCTCCTGCCGTGATCCCTACACGGAGGGGCCGGAAGAGGA |
| | | GGTCTGAGGAGGATGAGGAGGACACCTATTATACAAAGGACCTGCCCATCCACAC |
| | | CTGCAGCTACTGCGGAATCCACGACCCAGCCTGCGTGGTGTATTGTAACCGCGGCA |
| | | GGAAGAGACGGAGCCTGATCTCTAGGAGGGATGCCGAGGTGGTGCTGACAAGCAG |
| | | GGAGCTGGGCAGCCTGGCCCTGAACCAGTCTACAGGCCTGCCTACCCTGACCCTGC |
| | | CAAGAGGCCGGAAGCGGAGGTCTGACTTCTCTTCTAACGACTCCTCCACATGCACC |
| | | ATGGGCCTGGTGCTGACACTGTGGTCTGATACCCTGATCCACCTGGACGGCGATGG |
| | | AGGCTTTAGCAGAGGCAGAAAAAGGAGGTCCTCCAGCGCAGCCCCATCCGTGATG |
| | | GACCTGAGCTCCCGGGCATCTGCCGCACTGCACCCAGGCATCCCCTCCCCTAATTGT |
| | | ATGCAGTCTCCCATCCGC |
| 38 | Patient | ATGGATTGGACCTGGATTCTCTTTCTTGTTGCTGCTGCCACTCGCGTTCACAGTCCT |
| | PT1 | GACAGGAAGGCCGCCGTGTCCCACTGGCAGCAGCAGAGCTATCTGGGCAGCGGCA |
| | | TCCACTCTGGCGCCACAACCACCGCACCATCTCTGTCTGGGAGAGGCAGGAAACGG |
| | | AGAAGCGGCCAGTCTCCTACACTGCTGACATCTGCCTGTTTCTTCCCATCTGCAGCC |
| | | TTTACAGCCGAGTTTCTGTTTAGCCTGGGCTCCCTGTGTCAGCACCTGCGGGGCCGG |
| | | AAGAGAAGATCTCGCCTGGCCAGCGCACCTCCACAGAAACAGAAGCAGAAGCTGG |
| | | GCGAGAGGCTGTTCCCCCTGATCCAGGCAATGCACCCAACACTGGCCGGCAAAATC |
| | | AGGGGCAGAAAGAGGAGGTCTTGCGGCCACCTGTATGCCTACGATTGGATCTCTAT |
| | | CCCAGTGGTGTACACCCAGGTGGTGACCGTGGCCGTGTATAGCTTTTTTCTGACCTG |
| | | CCTGGTGAGGGGCAGGAAGAGACGCAGCGACTGTATGTCCGAGGATGGATATAAG |
| | | CTGGATAGGATCAAGGGAGAGTTCGAGTTCCACAATGTGACCTTTCACTATCCCAG |
| | | CCGCCCCGAGGTGAAGAGAGGAAGGAAGCGGAGGTCTATGGCCAACGATTCCCCT |
| | | GCCAAGTCCCTGGTGGACATCGACCTGCCTTCCCTGAGGGACCCAGCCGGCATCTT |
| | | CGAGCTGGTGGAGGTGGTGGGAAATAGGGGCCGCAAGAGACGCAGCTGGCTGGGC |
| | | ATCCGGATCTCTGAGTCTCCAGAGCCAGGGCAGCGGACATTCACAACCTTTAAGTT |
| | | TTGCCAGCTGCCATTCTACAAGTATTCCGAGTATAGAGGCAGAAAGCGGAGGAGCC |
| | | CCCCCCTGGCAAGACCAATCCTGCCAAGGGAGCGCGGCGCAATGGATAGAATCGT |
| | | GGAATACCTGGTGGGAGACGGCCCTCAGAACAGATACGCACTGAGAGGCCGGAAA |
| | | AGGAGAAGCACCTGCGTGTCCCTGGATGACGGGCTGTACGAGTGTAGCTGTGCCCC |
| | | TGCCTATTCCGGAAAGGATTGTCAGAAGAAGGATGGACCTTGCGTGATCAACAGGG |
| | | GCAGGAAGCGCAGAAGCCTGCACGTGGGCCTGTGTTCCGGCCCCTGTGAGATGGCC |
| | | GAGCAGCGGTGCTGCGTGGACTACGCCAAGAGGGGCACCGCCGGGTGTAAGAAGT |
| | | GTAAGAGGGGCCGGAAGCGGCGCTCCAGCTTCGAGAGCTACCTGAAAACAATGCC |
| | | ACCATACTTTCTGGGCCCACTGAAGAAGGCAGTGCGCATGATGGGAGCCCCAAACC |
| | | TGATCGCAGACAGCAGGGGCAGAAAGCGCCGCAGCCCTAAGTTCCACTCCTACGTG |
| | | TCTCTGCCATTCGGCTGCACAAGGGCCGTGGTGGAGTACAGGCTGCTGCAGGCCGC |
| | | ATACCTGGCCAAGCCCGGCGACAGAGGCCGGAAGCGCCGCTCCGGAGAGTACGTG |
| | | GATACAGGCAAGCTGATCGATAAGATCAATCTGCCTAATTTCCTGAAGGTGTATCT |
| | | GAACCACAAGCCACCTTTTGGCAACACCATGCGGGGCAGGAAAAGAAGATCTATG |
| | | TGCACCAACGCCAGAAGGGTGAGAAAGAGATGGCTGCCTAAGATCAACAGCATGC |
| | | TGCCCGAGGGAGTGGAGATGTACCGCACCGTGATGGGATCTAGAGGGAGAAAGAG |
| | | AAGAAGCCCAGACGGCCCACTGCTGCCATCTCCCGGCCTGCTGAGACTGCCAGGCG |
| | | GGCCTCGGCACGCCGTGTCTGGAGTGCCTGAGGTGTACCCCCCTGGCCCTAGAGGC |
| | | AGGAAGCGGCGGAGCGTGGGCACACAGGCCACCAAGTATAATAGCGAGGTGGTGG |
| | | ACAAGATGATCGAGGAGTTTCTGTCCAGCTTTGAGGAGAAGATCGAGAATCTGACA |
| | | GAGAGGGGACGGAAGCGCCGGAGCACAAACCTGCCTATCTTTAAGCTGAAGGAGA |
| | | GCTGCGTGCGCAGGAGATACTCTGATTTCAAGTGGCTGAAGAATGAGCTGGAGAGA |
| | | GACTCTAAGATCAGAGGCAGGAAAAGGCGCTCCCTGATCAGATGGTGTCTGGCACT |
| | | GCGCCCAAGCGACAGGCCAACCTTCGAGGAGATCCAGAAGCACCCATGGATGCAG |
| | | GACGTGCTGCTGCCCCAGGAGCGGGGCCGGAAGCGCAGATCCAAGTTTGAATGCG |
| | | GCTGGTGTAGCGGCGAGAGGAGGTGTACACTGCACCAGCACTGCACAAACCCAAG |
| | | CTCCCCATGGCTGGATTGGTCCAGCCACAACAGGGGACGGAAGAGACGGAGCCAC |
| | | GACCACACCGGCTTCCTGACCGAGTACGTGGCCACAAGGTGGTACAGCGCACCCGA |
| | | GATCATGCTGAATTCCAAAGGCTACACAAAGTCTATCGATAGGGGAAGGAAGCGC |
| | | CGCAGCTCCTCCGAGGCAACCGAGGTGAATCCAGAGTCTCTGGCAAAGGAGTGCCT |
| | | GGAGAAATCTCTGCTGCTGCTGAAGTTCCTGCCCACCGGCATCAGCTCCAGGGGAA |
| | | GGAAGAGGAGAAGCGTGAAGAAGGATAGATCCGCCTCTCACCTGGACCACAAACT |
| | | GGAGATCCGCCAGTGGAGGAGGTCCTTTCTGGTGTCCCTGTTTTTCTGCATCCCCGT |
| | | GCGCGGACGCAAGCGCAGGAGCCTGTTTACATTCGCCGTGGGAGTGAATATCTGCC |
| | | TGGGCTTTACCGCCTACAGGATCAAGAGAGCAGAGGGATGGGAGGAGGGCCCTCC |
| | | CACCGTGCTGCGGGGCAGGAAGAGGAGATCCATGAGCCAGGCCCCTGGCGCACAG |
| | | CTGTCTCCCCCAACCGTGTACCACGAGAGACAGAGGCTGGAGCTGTGTGCAAGGGG |
| | | CAGAAAGCGGCGCTCCATGTCTTCTAACAGCTTTCCCTACATCGAGCAGTCCGGAG |
| | | GAGGCGAGGCCACCGAGCTGGGCCAGGAGGCCACCAGGGGAAGGAAAAGAAGGT |
| | | CCCAGCCTTGTCGCAAGATGCTGCCTGATCGGTCCAGAGCCGCCAGATACAGGGCC |
| | | AATCAGAAGCTGGTGGAGTATATCGTGAAGGCAAAGGGAGCCGAGAGGGGCCGCA |
| | | AGCGGAGAAGCCCTGTCTTTCACGGCATGCTGGAGAGAGCACCAGCCGAGCCTTGC |
| | | TATCGGGCCCCAATGGAGAAGCTGTTTTATCTGCCACACGTGTGCTCTTATACCCGG |
| | | GGCCGCAAGCGGCGCTCCACCTACTCTTGCGTGGCCGAGAATATCCTGGGCACAGG |
| | | ACAGAGAGGCTCCGGCGCCGAGCTGGATGTGCAGTATCCCCCCAAGAAGGTGACC |
| | | ACCGTGAGAGGCAGGAAGCGCCGCTCCAGCCCCAGCGCAACAGAGGATTCCCTGC |
| | | AGCCTGCCACAGACCTGCTGAACAGGTCTGAGCTGCCCCAGAGCCAGAAGGCAAT |
| | | GCAGACCAAGGATGCCCGCGGCAGAAAGAGAAGGTCTGACCTGAGAAAGGTGCCC |
| | | GGCACAGACCCCGCCTTCGAGGTGTTTCCCCTGCACGATCACCAGTGTGTGCTGCG |
| | | GGGCCGCAAGCGCCGGAGCGACGCACTGGATTTCAAGAAGGATAAGGGCGCCTTC |
| | | TATCTGGTGTTCATCTGGACCATGACCAGAGGCAGGAAGAGAAGATCCGTGGATAA |
| | | CTACAAGCAGATCAAGGCCGACCTGGATCAGCTGAGGCTGCCTGTCGAGAAGTCCG |
| | | AGCTGTGGGTGGAGAAGAGCTCTAACTATGAGAATAGGGGCAGAAAGAGAAGATC |
| | | CAATGCCCCCGAGAATGGATATATGAAGTGCTCCAGCGAGGGCGACAACTACGGC |
| | | GCCACCTGCGAGTTCTCTTGCATCGGAGGCTATGAGCTGCAGGGCCGGGGCCGCAA |
| | | GAGAAGATCCGAGAGCGGAAGCAGCGGCAAGAGCTCCTCTTCCCTGGTGCGGGGA |
| | | GTCCACCTGAGCAGCTCCGGACCCGCCCTGCTGGCCGGCCTGGTGTCCCTGGACAG |
| | | GGGCCGGAAGAGGAGATCTTGGACAGAGAAGCGGGCCTCTTATGAGCTGGAGTTC |
| | | GCCAAGTCCACAACAAAGATCGCCGAGGCCGGCAAGGTGTCCATCCAGCAACAGT |
| | | CTCACATGAGAGGCAGAAAGCGCAGGAGCAAGATCCTGGCAAGGAAGAAGTCCAA |
| | | ACGGTCTGCACTGGAGAACTCCGAGGAGCACTCCACAAAGTACTCTAATTCCAACA |
| | | ACTCTGCCGGCTCCGGGAGAGGCAGAAAAAGAAGGTCCGCAGCAGCAGCCGCCGC |
| | | ACTGGCCGCAGCCCTGCTGCTGCTGAGGCGCGAGGACCCAGGACTGGGGGCCGGC |
| | | CCCAGCATGGCAGAGACAGAGGCCCTGCGCGGCAGAAAGAGGAGATCTCTGCCTT |
| | | CTTTTCCTCCTTTCGGCTCTATGAACTCTAACGCCGCAGGCAGCGTGAGCACCCAGG |
| | | CCAATACCGTGCAGAGCGGACAGCTGGGAGGACAGAGAGGCAGAAAGAGACGGT |
| | | CCAGCGGAGTGGTGCACCGGCTGGAGGTGGACGAGGACTTCGAGGAGCACAATGC |
| | | CGCAAAGGTGCATCTGATGGTGCACAATCTGGTGCCACCATTCCTGAGGGGCAGGA |
| | | AGCGGAGGAGCCTGCAGATCAAGCAGCAGGAGCTGTATAAGAACTTCCTGAGATT |
| | | CCAGGTGGAGGAGAAGAAGCAGCGGGAGGAGGCCGAGCGCGAGCGCCTGAGAAT |
| | | C |
| 39 | Patient | ATGGATTGGACATGGATTCTTTTTCTCGTTGCAGCAGCTACACGCGTTCATAGCGAG |
| | PT6 | CAGCAGTCTCTGTCTAGCGACGCCGATACCCTGTCTCTGACCGACTCTAGCGTGGA |
| | | CCCACCTATCCAGGACCCAAGGGGCAGAAAAAGGAGATCTGACCAGAACACCGAG |
| | | GATTACCTGTTTCAGGTGATCCTGGAGAAGCAGATCAGAATCCCCCACAGCCTGAG |
| | | CGTGAAGGCCGCCTCCGTGCTGAAGTCTAGAGGCCGGAAGAGAAGGAGCCTGAGA |
| | | GAGGATGGCCGGGAGCAGGAGATCCGCGACGGCGGCTCCCTGCGGATGATCCACT |
| | | TCGGCCTGGTGAGAGGCCGCAAGAGACGGAGCCTGCTGCAGCTGTGGCCAGTGCA |
| | | GCGGCTGACAGTGAAGAAGTCCACCGACAATCTGTTCAGAGAGCAGAAATCCTCCC |
| | | TGAACTACCTGCGCCAGAAGCGGGGCAGAAAGCGCCGGTCCCAGTGGCTGAGGGC |
| | | CGTGGCAGTGGAGAGCATCCACCGCTTCTGTATGCAGCCACAGCTGCTGAGATCCT |
| | | TCTGTCAGAGCTATGATATGAAGCAGCACAGAGGAAGGAAGCGGAGGAGCCTGGA |
| | | GGCCATCGTGCAGCTGTGGCGCATCCCATCCTTCGTGACCGAGCTGTATGTGAATT |
| | | ATGATTGTGATTATTATTGCTCCAATCTGTTCGAGGAGAGGGGGAGAAAGCGCAGA |
| | | TCCACCCCTCCCGGCCAGTCCCCAGGCTCCGCCCCCATCATCATCAACATCCACAG |
| | | AGCCCAGCTGACAAACCCAGAGGTGAAGTACAATTATACCGAGGACAGGGGACGG |
| | | AAGAGACGCTCTCCTTATGCCACAGTGGCACCCTCCACCCTGGCCCACCCTCAGGC |
| | | CCAGGTGCTGGCCAGGCAGCAGGCCCTGCAGCACGCCCAGACCCTGGCCCACGCC |
| | | AGAGGCAGAAAACGGAGAAGCCAGAGGGGCCTGCTGCAGCAGATCGGCGACGCA |
| | | CTGTCCAGCCAGAGGGGACGGGTCCCCCCAGCCGCACCCCCTGCACAGCCCAGGGT |
| | | GCCAGTGACCAGAGGCAGAAAGCGCAGGTCCATGTTTAGGTCTAAGAGATCCGGC |
| | | CTGGTGAGGAGGCTGTGGCGGTCCGGCGTGGTGCCCGATCGCGAGGAGGGAGGAA |
| | | GCGGCGGCGGCGGGGGAGGCCGCGGCAGAAAGAGGCGGTCCCTGGAGATCAAGGT |
| | | GAGCCCACCCGAGGGCGCAGAGACACGCAAGGTGAGAGAGATCGGCCCTCTGTGC |
| | | GGACGCAGAAGGCCACGGGTGAGAAAGTCCAGGGGCAGGAAGAGAAGGTCTACC |
| | | CCTAACTTTATCATCCCAGCCCAGAGAGCCGAGCCTATGAGGATCGTGCGCCAGCC |
| | | TATGCCACCCCCCGGGGACCTGGAGCCTCCTTTCCAGCCTAGAGGCAGAAAGCGGC |
| | | GCTCTGGCACAGGCGCCCTGGGCCTGCTGCTGCTGTTCCTGCTGGGCTATGGACTG |
| | | AGGAGAAGGCCTCAGAAGGCCGAGGAGCCTGCACCTCAGGCAGGCGGCCGGGGAC |
| | | GGAAGAGAAGATCTGACAGCCACGTGTCCGAGGTGCTGCAGGAGAACTATCGGCT |
| | | GAAAAACAAGCTGGAGGGACTGATCAGCGAGAAGAATGAGCTGAAGATGAAGAG |
| | | CGAGAGGGGACGGAAGCGGAGATCTGTGTCTGCATACCAAGTGCAGAGCCCAAGC |
| | | TGGATGCAGCCTCAGCCATGTATCCTGCAGCACCCTGGCGCCGTGCTGACCCCTTCT |
| | | ATGGAGCACACCAGGGGCCGGAAACGGAGGAGCGGCAGGTTCAGCATCGTGAAGA |
| | | AGTGCATCCACAAGGCAACCAGGAAGTACGTGGCCGTGAAGTTCGTGTCCAAGAA |
| | | GATGAAAAAGAAGGAGCAGGCCCGCGGACGCAAGCGCCGGTCTGGCATCCTGCGC |
| | | GACCCAGGCTCTGAGATCGAGGACAGACAGTATAGAATCGATCTGCAGTCCATCAA |
| | | TATCGGAACCGCCCAGTGGCACCAGCTGAAGAGAGGAAGGAAGAGAAGGTCCGAC |
| | | GGAACACAGGTGCTGAAGATCTACGTGAAGCAGACAGAGTCCCACTACATCCTGGT |
| | | GTCTTGGAAGGTGAATTCTAACGTGATGACATCTAATCTGAGAGGCCGCAAGCGCC |
| | | GGAGCCCCCCAGGATACATCCCCGATGAGCTGCACCAGGTGGCCCGGAACGGCCT |
| | | GTTTACATCTATCAACTCTGAGGGCGAGTTCATCCCCGAGAGCATGGATAGGGGCA |
| | | GAAAGAGACGGAGCCCAAAGTTTCGGGGAGTGAAAATGATCCCTCCAGGCATTCA |
| | | CTTCCTGCACTATAGCAGCATGGACAAAGCCAATCCTAAGGAGGTGGGCCCACGCA |
| | | TGAGAGGCAGAAAGAGAAGATCCAATGTGAGCAGCGCCGAGGGATGGCACGTGAA |
| | | CGTGACCCTGTCTATCAGGCCCTCCACAGGCACCGGCGTGATGCTGGCACTGGTGA |
| | | GCGGAAATAACAGGGGCCGCAAGAGGAGATCTTGTCAGAGGTCTTTTAGGACCTTT |
| | | CAGGCCCTGAAGAAGCACCTGGAGGCCTCCCACCTGGAGCTGAGCGAGGCCGATA |
| | | TCCAGCAGCTGTACGGCGGCCGCGGCAGGAAGCGCAGATCCGACTCCGTGGACTTT |
| | | CGCCTGAGGGTGAGCGAGCCCAAGGCAGTGTTCGCCAAGGAGCAGCTGGCATGCA |
| | | GGGAGGTGCAGGCAGAGGTGGGCGCCTCTAGAGGCAGGAAGAGGCGCAGCGATAT |
| | | CAAGGATGGAAGATTTCCTTACGGATCTACCCAGGATTACCTGAAGCCAATCATCC |
| | | TGATCAAGCTGGTGCAGCTGGGCATGGCAAAGGATGACAGGGGCAGAAAGAGGCG |
| | | CTCCGAGAACTACCGGAACCTGGCCTTTCTGGGGATCGCACTGTCTAAGCCAGACC |
| | | TGATCACATTCCTGGAGCAGGGCAAGGAGCCCTGGAATATGAAGCAGAGGGGCAG |
| | | GAAGCGGAGATCCTGGAATAGCCTGGGCAAAATGTCTCGCGAGGAGGCCATGAGC |
| | | GCATACGTGACCGAGATGAAGCTGGTGGCCCAGAAGGTGATCGATACCGTGCCCCT |
| | | GAGAGGGCGGAAGCGCAGGTCCACATTTCAGTGCGTGGTGTCCCCCTCCGACGTGG |
| | | CAGTGGTGTGGTTCTGGGATGGAGCCCTGCTGCAGCCCAGCGAAAAGTTTGCCATC |
| | | TCCCAGAGCAGGGGCAGAAAGCGGAGGTCCCACGTGGATCTGTTCGACCTGGAGA |
| | | GCGGAGAGTATCTGTGCCCACTGTGCAAGAGCCTGTACAACACAGTGATCCCAATC |
| | | ATCCCACTGCAGCCTCAGCGCGGCAGGAAGAGGAGATCTAGCGAGATCATCAGCA |
| | | AGCTGTACATCCCCAAGAGGAAGATCATCTCTCCAAGAAGCATCAAGGACGTGCTG |
| | | CCACCAGTGGAGGAGGCCGTGGATAGG |
| 40 | Patient | ATGGATTGGACTTGGATTCTCTTTCTCGTTGCCGCAGCTACACGCGTTCACTCACCT |
| | PT2 | GATCGGAAAGCCGCAGTGAGCCACTGGCAGCAGCAGTCCTACCTGGGCAGCGGAA |
| | | TCCACTCTGGAGCCACAACAACCGCACCATCTCTGTCTGGCCGGGGCAGGAAGAGG |
| | | AGGTCCGGCGATAACACAGGCGAGCAGGTGGCCGTGAAGTCCCCTAAGCCTGAGA |
| | | GCGGAGGCAATCACATCGCCGATCTGAAGAAGGAGATCGAGATCCTGAGGAGAGG |
| | | GAGAAAGCGGAGATCTAAGGATCACGGCGTGAACTCCTTTCTGGTGTACATGGCCT |
| | | TCAAGGACCTGTTTCAGCTGACAGATTGTCAGATCTACGAGGTGCTGTCCGTCATCC |
| | | GGAGAGGCAGAAAGCGGAGGTCTAACACAGACGGCAAAATCGAGTTCATCTCTAC |
| | | CATGGAGGGCTACACCTACCCAGTGTATGGCGTGCAGTGGCATCCTGAGAAGGCAC |
| | | CCTACGAGTGGAGGGGCCGCAAGAGGAGATCCAATAATAGCGTGCTGCAGAAGCA |
| | | GCAGCTGGAGCTGATGGATACCGTGGACAATCTGGTGAACCTGTGTACCAAGGAGG |
| | | GCGTGCTGCTGAAGGGAGGCCGCGGCAGAAAGAGACGCAGCGACTCCAACCGGGA |
| | | GAAGGACATGGGACTGTTCGAGGTGTTCTCTCAGCACCTGCCCACCACCGAGCCAG |
| | | TGGACAGCTCCGTGAGCTCTAGCATCAGCAGAGGCCGCAAGCGGCGCAGCCTGAA |
| | | GGCCGAGGTGTTCACAGAGATCCTGAATTACATCTATTCCAGCACAGTGATCGTGA |
| | | AGAGGCAGGAAACCGTGACCGACCTGGCCGCCGCCGGCAGAGGCAGAAAGAGAC |
| | | GGTCCCACCCAAATAACGTGGTGTCCATCAAGTACTGCAGCCACTCCGGGCTGATG |
| | | TTTAGCGTGTCCACCTCCTACATCAAGGTGTGGGATATCCGCGACAGCAGGGGCAG |
| | | AAAAAGAAGGTCCACAAAGAACAAGCGGAGAAAGGAGTATTTTCTGGCAGTGCAG |
| | | ATCCGGAGCCTGGAGGAAAAGATCAAGGTGAAGTCTACAGAGGTGGAGATCCTGG |
| | | AGAGAGGAAGGAAGCGCCGGTCCCTGGTGAAGCCACAGAAGGAGAATGAGCAGG |
| | | CCGAGAAGAAGAACATCTCTCTGGCCTTCTTTCTGTATGACCTGCTGTCCCTGATGG |
| | | ACAGAGGCTTTAGAGGACGGAAGAGGCGGTCTGTGGATATCGTGATCCTGGGCCTG |
| | | AGCTACGCCAACCTGCAGACCGGCAGCGGAGTGCTGAGCGATTCTCTGAGCGGCG |
| | | GAATGCAGCTGCTGCCAGATAGGGGCAGAAAGCGGCGCTCTATCACCAAAATCAC |
| | | CAGGAGACGCCACGAGAATCTGCCCCACGGCGTGGCAAGCGTGAAAGAGTGGTTC |
| | | AATTACGTCACCGCAACACGCAACGAGGAGAGAGGACGCAAGAGGCGGAGCGCCG |
| | | TGCTGAAGGTGGGATGGCTGTTTCCTCTGTCCGAGGTGCCTAACTTTACACTGCTGA |
| | | TGGACGGCTGTGGCTGCTGGAGGCTGAAGGAGGATCAGCGGGGCCGCAAGAGAAG |
| | | ATCTCTGGTGGCCGCCCTGCTGCTGGGCTCCGTGTGCTGCGGCTCCGCCCAGCTGCT |
| | | GTTCAATAAGACCAAAAGCGTGGAGTTCACCTTCAGGGGCCGGAAGAGGCGCTCC |
| | | AGCCACGTGTTTGGAACCAACACCAGCTCTCTGGAGCTGTTCCTGATGAACGGCAA |
| | | GATCAAGGGACCCTGTTGGCTGGAGGTGAAGTCTCCACAGCTGAGAGGAAGGAAG |
| | | AGAAGGAGCTCTCCCTACGATCACACACCAGGGATGGCAGGCTCTCTGGGCTACCA |
| | | CCCCTACGAGGCCCCCCTGGGAAGCTATCCATATGGCGACCCAGCCTATAGGAGGG |
| | | GCAGAAAGCGCAGATCCATGACAGGGTCCTCCCTGTCCCTGGCCCACCTGCTGATC |
| | | ATCAGCGGCCTGCTGTGCTATTCCGCAAGGGGCAGGAAGCGGCGGTCCGCCGAGG |
| | | AGGGAGACGAGCTGCAGCAGAGATGTCTGGACCTGGAGAGACACCTGATGCTGCT |
| | | GAGCGAGGAGAAGCAGTCCCTGGCCCAGGAGAATGCCAGGGGCAGGAAGCGCAG |
| | | ATCTACCATGTATCACGGAGTGCCTGTGGTGGGCATCCCTCTGTTCGGCGACCACTA |
| | | CGAGACAATGACCCGCGTGCAGGCCAAGGGAATGGGCATCCTGCTGAGGGGCAGG |
| | | AAGAGACGCTCCCAGCCTGCATCTCACATCCTGAATAGCGAGAAATTCCCTTTCTC |
| | | CAAGTACGAGGACTGCCTGGAGCAGCAGATGCCCAGCCTGCGCGAGTCTCCAATGC |
| | | GCGGCAGGAAGAGACGGTCTGTGGAGAAGAGCCCCGCAAAGAAGAAGGCCACCA |
| | | AGAAGGCCGTGGGCGCCGGAGCAGCCAAGAGAAAGGCCACCGGCCCACCCGTGAG |
| | | CGAGCTGATCCGCGGCAGAAAACGCAGATCCCTGCTGGCAGCCGGCCCATCCGCCG |
| | | CCGCCGCCAAGCCAAATATCCCTAAGGTGCTGCTGCCATTTACAAGGGCAACAAGA |
| | | GTGAACTTTACCCTGGAGAGGGGCAGAAAAAGGAGGAGCCTGGCACCCCAGGACA |
| | | TGAGAGTGTTTTACGCACACACCGCCAGGGCCCAGCTGCCCGTGTGGAACAATTGG |
| | | ACCCAGAGAGCAGCCCGCTATGAGTGCCGGGGCCGCAAGCGGAGGTCCCCTGCAC |
| | | CTCTGAGACGCCCCCTGGCAGTGCTGCACGCCCCAGTGCCAACAACCGCACTGGCA |
| | | AGACTGTCCAGGCCTCAGCGGGCATGTCCATCCTCTAGGGGCAGAAAGAGGCGCA |
| | | GCAAGCACTGGTTCGTGCTGACAGATTCCTCCCTGAAGTACTACAGAGACTCTACA |
| | | GCAGAGGGCGCCGACGAGCTGGATGGAGAGATCGATCTGCGCAGCAGAGGCAGGA |
| | | AGAGGCGGAGCGTGAAGTTTCTGAGGAAGCCAGGCGGAAATCTGGGAAAGGTGTA |
| | | TCACCCAGGCTCCATGCTGTCCCTGGCACCTACAAAGGGCCTGCTGAATGAGCCTA |
| | | GAGGCCGGAAGCGCAGGTCTGATTATCAGGTGTACCTGAACGCCAGCAAGGTGCC |
| | | CGGATTCGCCGATGATCCTACAGAGCTGTCCTGTCGGGTGGTGGATACAAAGAGCG |
| | | GCGAGGCACGCGGGAGGAAGAGGAGGAGCCAGCGGGATGATCCTGTGCTGGAGAC |
| | | CAGCCACCAGAACTTTAGGCGCTCCAGATATCAGGAGGCCGCATCTCCAAGGGAG |
| | | GCCCTGATCCGCCTGCGCCGGGGCAGGAAGCGGAGATCTCACCTGCTGAATTATGT |
| | | GTGGCCAAACGTGTTCGAGACCTCTCCACACGTGATCGAGGCAGTGATGGGCGCCC |
| | | TGGAGGGCCTGAGAGTGGCCATCGGCAGAGGCAGGAAACGCAGGAGCAGCAGGA |
| | | CATCTATGGACGATCTGAAGGCCTTCACATCCCTGAGCCTGTATATGAAGCCTCTGC |
| | | GCGGACGCAAGCGGAGATCTTGCGCCGCCTATGGAGGCTATATCAACTGCATGGCC |
| | | GTGCTGATGGAGAGCAACGCCGATCCTAATATCCAAGACAAGGAGGGACGCACAG |
| | | CCCTGCACAGGGGCAGAAAGAGAAGGTCTGATAACCGGCTGCCCCCCAAGAAGGT |
| | | GCCCGGATTTTGCAGCTTCCGCGACGGCCTGAGCTTCCTGGTGCACTGCTGTAACGT |
| | | GATCATCACAGCCCAGAGAGGCCGGAAGAGGAGATCCCCATACTCTACCGCCGTGT |
| | | CTACCTCTGGCTGCGGAGAGCACCTGGTGCACACCATCCTGGCAAGAGAGTGTTCC |
| | | CACGCCCTGCAGGCAGAGGACGCCAGAGGCCGCAAGAGAAGGTCCGGCGTGCTGA |
| | | AACAGTATCAGATCAAGGGCCTGGAGTGGCTGGTGTCCTGTACCACAACAACCCGG |
| | | GGCAGAAAGAGGAGGTCCTCTAAGTCTCCCTCCAAGAGAAGCAAGTCCCAGGACC |
| | | AGGCAAAGAAGAGCAAGAGCCCAACACTGAGAAGAAGGAGCCAGGAGAAGATCG |
| | | GCAAGGCAAGGGGAAGGAAGCGGAGGAGCTTTTCTCTGTGGAAGGGCTTTACACC |
| | | CTACTACGCCTGCCTGGGACCTCACACCGTGCTGACCTTCATCTTTCTGGAGCAGAT |
| | | GAACAAGGCATATAAGAGAGGCCGCAAGAGGCGCAGCGATTCTTTCCTGGTGGAG |
| | | CGGGGAAAGCTGCTGCACAAGCGGGATAACGACAAAGTGGATACCCAGGAGGAGA |
| | | ATTTCCTGCCAAAGTACCAGCGGGTGAGGGGCAGGAAAAGAAGGAGCTTTCGGAC |
| | | CTACCACCAGATGGTGCTGCACTCTAGGGTGCACCGCAGAGCCCGGAGAGAGATG |
| | | GACTCTGACGGAGACAGGGCCGCCAGAGCCCGCTGTAGAGGCAGGAAGCGCAGGA |
| | | GCCTGGAGGAGAATATCAATTCTGCCCTGGACATCAAGGTGCTGACCCAGAGCTCC |
| | | CAGTCTGCCGTGAGGGGAGGCCCCGGGAGATCCACCATGCCCACCAGAGGGCGGA |
| | | AGAGACGGTCCGGCGCCAGCGTGAACGCCCCCCCAGACCCTTGCAAGCAGAGCCC |
| | | AGTGAGACTGGCAGCCGGCTCTGGCCTGGCCTGCTTCCTGCTGTGGCAGCTGCAG |
| 41 | Patient | ATGGATTGGACATGGATTCTCTTTCTCGTTGCTGCAGCTACTCGCGTTCATAGCCAG |
| | PT3 | TCCTATCTGGACAGCGGCATCCACTCCGGCGCAACAACCACAGCACCTCCACTGTC |
| | | TGGAAAGGGAAATCCTGAGGAGGAGGATGTGGATACATCCCGCGGCAGGAAGAGA |
| | | CGGTCTTGGATGGGCGACTTTGTGACAGCCTGGATGGTGACAGATATGATGCTGCA |
| | | GGCAAAGCCCTACCCAGACTGGGGGAAGTCTGCCAGGGCCTTCTGGAAAAGGGGC |
| | | AGGAAGAGACGCTCCCCCACCGGCAGACCATACAATGTGGATACCGAGTCTGCAC |
| | | AGCTGTACCAGGGCCCCCACAATACCCTGTTTGGCTACTCCGTGGTGCTGCACTCCC |
| | | ACCGGGGCAGAAAGAGGCGCAGCGAGAAGCTGGCAGCCGAGGAGCAGTTTCAGGC |
| | | CCTGGTGAAGCAGATGTATCAGACACTGCAGGACAAGACCAACCAGATCGACCTG |
| | | CTGCAAGCCGAGAGGGGCCGCAAGAGAAGATCCAACGAGAAGATGGATTCCATCG |
| | | TGCAGTGCCGCGCACTGAACATCGCAGTGGACCTGGGCCAGATCCAGTACCTGTTT |
| | | TCTGACAAGACAGGCACCCTGAGGGGCAGAAAGCGGCGCAGCGAGGAGCTGCCCG |
| | | TGGTGGGACAGCTGCTGAGACTGCTGCTGCAGCACACCCCTCTGAGAACACACATG |
| | | CTGACAAACGCCATCCTGGTGCAGCAGATCAGGGGGAGGAAGAGGAGGTCTAACC |
| | | TGCCCCTGCTGCAGATCGAGCTGCTGCGGTCTGCAATCAAGGCCCTGAGGCCTGGC |
| | | CGCATCCTGGTGTACAGCACATGCACCCTGAGCAAGGCAAGAGGCCGGAAGCGGA |
| | | GAAGCGGATTTCAGTGTGCCGCCCCACTGCTGATCCTGACCAACAAATTCTGCTTCC |
| | | TGAGCACCTACGTCTTTGTGAGCACCTTCCTGACCGGCAAGGAGCTGAGAGGCAGG |
| | | AAACGCCGCTCCGTGACCGCAGCCTTCCTGGTGAGCTTTATGCCATACCACATCCA |
| | | GCGCAATATCCACCTGCACTTTCTGCACAATGAGACAAAGCCATGTGATTCTGTGA |
| | | GGGGACGGAAGAGAAGGTCTGCCAGGCCCGAGCTGCTGCAGAGCCACTTCATCCC |
| | | TACCATCGGCCGGCTGCGGAAGAAGGCCGGCAAGGTGGTGAGCGAGGAGGAGCAG |
| | | CTGAGGCTGAGAGGCAGAAAGCGCAGATCTGACCGGGTGGATGCCCAGGACGAAA |
| | | CCGCCGCCGAGTTCACCCTGCGGGTGAACCTGACCGCCGCCAGACTGGGCCAGCTG |
| | | GAGGGACTGCTGCAGGCCAGGGGCCGCAAGCGCCGGTCCCTGGTGCAGTCTAGAC |
| | | AGAACCTGGTGGCCGAGGAGCGCCTGTCCAGCGGCATCCTGGGCACAATCGGATTC |
| | | GGACGCAAGTCCCCACTGTCCAACAGAAGAGGCAGGAAGAGAAGGAGCATGGCCT |
| | | TTAGAACCATCTGCGTGCTGCTGGAGTATCTGTTCGTGCTGTCCGTGAGGGGCAGG |
| | | AAACGGAGATCCTTTGAGAAAGGACGCATCTATACATTTATCGGAGAGGTGGTGGT |
| | | GAGCGTGAACCCATATCAGCTGCTGAACATCTACGGCAGGGATACAATCGAGCAG |
| | | AGAGGAAGGAAGCGGAGAAGCTGGCCCGCCGTGTTCGCAAGAGTGAGCGTGTTCG |
| | | TGGATTGGATACACAAGGTGATGAGGCTGGGCCGGGGACGGAAGAGGAGGTCTGT |
| | | GTGCGTGAGCTCCGACACCCTGGATGGAAATTCCAGCAGCGCCGGCTACTCTTGCA |
| | | ACAGCCCCGCAAAGGTGTCCACCACCACAGATTCTCCAGTCAGAGGCAGGAAGAG |
| | | GAGAAGCGTGGAGGACGCCGTGGTGTTCTCTACAGAGCTGGAGGCCAGCGCCATCT |
| | | CCCCAAGGGTGGTGGTGCCTCTGGCCGAGACACACTGTGAGGAGCAGGGCAGAGG |
| | | CAGAAAGCGGAGATCTGCCGCCCAGCCCTACTCTAATCTGTCCAATCTGGACGTGC |
| | | TGAATCAGGTGGTGAGGGAGAGAGACACAAAGCTGCCAAAGCCTCAGCTGGAGCA |
| | | GCCTCGGGGCAGAAAGAGAAGAAGCGAGGTGAAGAGAAGCCAGCGCCTGCGCCG |
| | | CGGACCTTCCTCCCCTAGGCGCCCATATCAGGAGATGGAGTATGAGAGGAGAGGA |
| | | GGAAGAGGCGACAGAAGAGGCAGAAAGAGAAGGAGCGCATCTAGCCTGAATGAG |
| | | AACGTGGACCACAGCGCACTGCTGAAGCTGGATACCAGCGTGTCTGAGAGGGTGTC |
| | | TAGGTTCGATTCCAAGCCTGCCCCTAGAGGGCGGAAGCGGAGGTCTTGTCACCCAG |
| | | GCCCAAGGGACCTGCTGCTGCTGCACCTGCTGCAGAGAAAGGACAATATCGACCGC |
| | | GGCCGCAAGCGGAGAAGCGACCTGCTGCGCAGCGTCCTGCAGCAGAGACTGATCG |
| | | CCCTGGGCGGCATCATCGCCGCACGCATCAGCGTGAGAGGCAGAAAACGCCGGTC |
| | | CTATACCATGCAGAAACACCTGGAGATGAACCCCCACTTCGGCTCCCACCAGTATC |
| | | AGATCCTGCCCCTGCACTCTCAGATCCCAAGGGAGGAGCAGCGGAGAGGCCGCAA |
| | | GCGGAGGTCCTGTTCTTCTTCTCTGATGGCCCTGCAGAATGCCTACCAGGCAGTGCA |
| | | CAGCGGCCAGTGCCCCGCCGCCATCGTGGGCGGCATCAACGTGCTGCTGAAGAGG |
| | | GGGAGGAAGCGGAGGAGCTTCAGGCTGAACATCCAGGGCCTGGAGCGGACCCTGC |
| | | TGGGCTTCGTGTCTAAGACCCTGGACTCCGCATCCGCCCAGTTTGCAGCATCCGCCC |
| | | TGGTGAGGGGAAGGAAGAGGCGGAGCATCAATCCTCAGGGACTGTCCGATCCCTC |
| | | CCCAATGAGCGATCCAGTGCTGACCCAGGATATCAGCCCTCCTGCCCAGGGAGTGG |
| | | ACCACCGGCAGGTGAGAGGCAGGAAGCGCCGCTCCCTGGGCCCACACGAGGAGGA |
| | | TCAGGTGGTGTGCGGATTCAAGAAGCTGTCTGAGTGGGGCCTGTGTTTTCACCCCTC |
| | | CACAGATACCCACAAGAAGCTG |
| 42 | Patient | ATGGATTGGACTTGGATTCTTTTTCTCGTTGCTGCTGCTACTCGCGTTCACAGTCAG |
| | PT5 | GAGAAGGCCTTCTTCGCACAGCTGCAGCTGGGAGAGGAAACCGGAGAGTTCCTGC |
| | | CTATCCAGCCCGCCCAGCACATCCAGTCTGAGACCTCCGGAAGGGGCAGAAAAAG |
| | | GCGGAGCTCTTATCTGGATTCCGGAATTCACTCCGGCGCCACCACCACAGCACCCT |
| | | TCCTGTCTGGAAAAGGAAATCCTGAGGAGGAGGACGTGGACACAAGCCAGCGCGG |
| | | AAGGAAGCGGCGCTCCGGATGTTGCCCACCCGGCCACGGCTCTAGGAGCAGGCCC |
| | | CCAGCCCAGCCCGCCTGCTCTAGAGCAGAGCAGGATATCCTGTTTCTGCTGAGAAG |
| | | CCCAAGGGGCAGAAAGAGGAGGTCCCTGGTGTCCCTGCAGCCAGCCCAGATGCAC |
| | | AGGTTCTGGAGAGGGCTGTCCCTGCACCTGGACTCCGTGGATGCCGTGTATGAGAG |
| | | AACATCTGACCACAGAGGCAGGAAAAGGAGAAGCATCCAGCTGCCAATCATCCAG |
| | | CTGAGAAAAGTGTGGGCCGAGGCCGTGAGATACGTGTCTGGCCTGAAGGAGGATT |
| | | ACTCCAGACTGTTTCAGGGCCAACGCGGAAGGAAGAGGAGATCTATCTTTACCGGC |
| | | CAGGCAAATGTGGTGAGCGAGTATCTGCTGGCCTACCTGTTCCTGTCCCTGGTGGC |
| | | ATTTTTCAGACTGATCGCCTTTCTGAATCTGCGGGGCAGGAAGAGACGGTCTGGCA |
| | | GGAGATGTATCCGGGCCACCCCCAGAGCCTGCAGAGCAAGATGTGCCACATGCTGC |
| | | GTGCGGTCCAGCGCCGCCACAAGCCCCTGTTGCGGATGCAGAGGCCGCAAGAGGA |
| | | GATCCATGGCCTTCGCCCTGGCCATCAGAGACACCGCAACCTATGCCAGACAGCTG |
| | | ATGTTTTCTACCACCCTGCTGATCGTGTTCTTCACCGTCTGGGTGTTTCGCGGACGG |
| | | AAGCGCAGGTCTTGGCGGCCCCTGCTGAGAGGCCCCAGGCTGAGCCTGCACACCGC |
| | | CGCCAAGGCCGCCGCCACCGCCACAGAGACAACATGCCAGGATGTGGCAGCAACC |
| | | CGGGGCCGCAAGCGCCGCAGCGAGGCCCTGGCCGAGAACCCAGGCCTGGTGAACA |
| | | AGAGCTGTTATGAGGTGGGCTGGCTGATCAAGATGACACTGTCTAATCCTAGCGAG |
| | | CTGGACGAGCGCGGCCGCAAGAGACGCTCCCCTTCCGGCTCCTTCAGCACCCCAGG |
| | | CTCTGCAACATACGGCAGGTATAAGCCCAGCCCTGAGCGCTACACCGCAGCCCCAC |
| | | ACCCACTGCTGTCCCTGAGGGGCAGAAAGCGCAGGAGCCTGCAGACAAGACTGCA |
| | | GCCTGAGGAGAGCACAGAGACCCTGGACTCCAACTACGTGGTGGGCCACGTGCTG |
| | | AATAGCCGGAAGCAGAAGCAGCTGCTGCGGGGCAGAAAAAGAAGGAGCCTGGAG |
| | | GTGTCTCCCTTTTTCAAGTGTCACGAGGTGATCGGATCTTCTGTGCTGTTCATCCAC |
| | | GATAAGAAGGAGCAGGCAAAGGTGTGGATGATCGACAGAGGAAGGAAGCGGCGG |
| | | AGCGTGGCACAGCCAAAGAAGAAGCTGGGCAGGAGACTGGTGTTTCACAGCAACC |
| | | CCCCCAGCGGAAATAAGCAGCAGCTGCCCCTGGTGAATGAGACAAAGAGGGGCCG |
| | | GAAGAGGAGGTCTGTGCACATCAGGCGCAAGCACAGCCACCAGTACAGCTATCTG |
| | | TGCGAGGTGTGTAAGTATTATACAGTGACAAAGGGAGACATGGAGAGACACTGCG |
| | | CCAGAGGACGGAAGCGCCGGTCTTGTCACGTCGGCTCTGAGGAGACAAGGGCAAG |
| | | AGTGCCAGTGCACGACCTGCACGTGGGCATCACCAAGAGACTGAAAACAATGGAG |
| | | GTGCTGGAGGGCAGAGGCAGAAAGAGAAGGTCTGTGACCGACTACACCTTTGAGG |
| | | ATTGTCAGCTGGCACTGGCCGAGGGACACCTGCGCCTGCCTGCCGACACATGTCTG |
| | | CTGGAGTTCGCAAGACTGGTGAGGGGCCGGAAGCGCCGGAGCGAGATCCACCTGG |
| | | AGATCTTTGAGAGCGCCAGCGCCGGCACCCGCCTGCTGCTGGACCCCGCCACCGAC |
| | | CCAGATATCAACATCAACTCTATCAAGGATCGGGGAAGGAAGCGGAGATCCGTGT |
| | | ATGTGAGCACCCAGTTCCCTAAGTACATCCAGGATATCGTGGCAGCCACACTGAAG |
| | | CTGCCTGCCAACAAGGTGATGTGTCACGTGAGACGGGTGAGGGGGAGAAAGAGAA |
| | | GGAGCCTGACCCAGAGCAAGAGCTCCCCAAAGAGGGGCTTCTTTAGGGAGGAGAC |
| | | AGACAGGCTGATCAAAAACCTGCTGGGCAAGAGGATCTCCAAGCTGATCAGAGGC |
| | | CGGAAGAGGCGCAGCAACAACATGATCCACCAGGTGCCAATCAAGTCCCTGCCTC |
| | | AGGAGTGGCTGTGGTGCGAGATGTGGTGCGACGACGCATCCAAGAAGCGGGCAAA |
| | | GACAAGAGGACGCAAGCGCCGCTCTCCTGAGAATATCGAGATCACCGTGACCCTGT |
| | | TTAAAGATCCTCACGCCGAGGAGTTCGAGGGCAAGGAGTGGACATTTGTGATCGAG |
| | | AATGAGTCCCCAAGGGGAAGGAAGAGACGGTCCGCCGCCTCTAGACCACGGCTGG |
| | | ACCCTTGGAAGCTGGTGTCCTTTTCTCGCACCCTGACCATCAGCCCCCCTAGCCGCC |
| | | CAGATACCCCTGAGTCCCCTCGCGGGAGAAAGAGAAGATCCTCCAGCAAGACAAC |
| | | ATCCGTGCTGCAGTGGGCCGAGAAGGGCTATTACACCCGCTCTAACAATCTGGTGA |
| | | CACTGGAGAATGGCAAGCAGCTGACCGTGCGGGGCAGGAAGCGCAGAAGCCTGGA |
| | | CTGGCAGCCTAGGGAGAATCCATTTCAGAATCTGAAGGTGCTGTCCGTGAGCGATC |
| | | AGCAGCAGAATTTTCTGGAGCTGTGGAGCGAGATCCTGAGAGGCAGAAAGCGGCG |
| | | CAGCGAGAGATGGGAGTGCACCACCTACTGGTATATCTTCGTGTTCTGCTCCGCCC |
| | | ACCCTGCAGTGACCGAGATGGCCCTGTTCGTGACAGTGTTCGGCCTGCGCGGGAGG |
| | | AAGAGGCGGTCCTTTTACAAGAAGTGCGCACAGCTGACAGTGAATCTGACAAGATT |
| | | CCCAGAGACCGTGCCTGGCGAGCTGGTGGTGCCCGTGGCAGGCTCCTGCGTGGTGA |
| | | GAGGCCGCAAGAGAAGGTCTAACAATGTGGACCTGATCCTGGCCACCCCACCATTT |
| | | TCTAGACTGGAGAAACTGTACTCCGGCGAGGTGGTGAGAGGCAGGAAGCGCAGGT |
| | | CCAAGACCATGAAGGATTTTGTGCGGAGGAGGTGCTGGGCAAGGAAGTATAAGCT |
| | | GGTGACCTCTGGACCTTGGCTGGAGGTGCCCCCAATCGCACTGAGAAGGGGCAGA |
| | | AAGCGGAGGAGCGCCAGGAGAATGGCCTTTCTGGCCAAGAAGGGCTACAGGCACG |
| | | ATTCTTCCACAGCAGTGGTGGGGAGCCCCAGGGGCCACGGCCAGTCTAGAGAGAC |
| | | CCGCGGCAGGAAGCGGAGATCTTGGCGGCGGGCACAGAAGCTGCATTTCCTGCAG |
| | | AGGGGCGGGCTGAAGCAGCTGAGCGCCCGGGTGAGGCCAGAGCTGAAAACCAAGC |
| | | AGCAGATCCTGAGGGGCCGCAAACGGAGGAGCTCTGGCCGGCTGGATCAGCTGCT |
| | | GTCTCTGTCCATGGTGTGGGCCAACCACGTGTTCCTGGGATGTAGCTACAATAAGG |
| | | ACCTGCTGGATAAGGTGATGAGGGGCAGGAAGAGGCGCAGCGAGCTGGGCTCCCT |
| | | GATCCGCCAGCTGTCCCTGTGCTCTGTGAAGCTGTACGCCGATCCATCTGTCCCAGA |
| | | TGTGGTGATCGACATCCTGCAGCAGATCAGAGGCAGGAAGAGAAGAAGCCCTGAG |
| | | CTGTCTATCAGCAGCCTGACATCTCCTATCCCAGAGAACAGCACAGAAACCGAGGT |
| | | GGCCCTGTTCAGGATCAGAGACAGAGATTCCGGCGAGCGCGGCCGGAAGCGCAGA |
| | | AGCACCTCCGAGCTGCTGGGCGAGGGCGCCTACGCCAAGGTGCAGGGCGCAATGT |
| | | CCCTGCAGAACGGAAAGGAGTACGCAGTGAAGATCATCGAGAAGCAGCGCGGCAG |
| | | GAAGAGACGCTCTGATTGGGAGGGCAACAGGGCATATAGCCAGTACGACAAGTTC |
| | | CACATCGGAAACGAGAAGCAGAACTATAGACTGTACCTGAAGGGCCACACAGGCA |
| | | CCCGGGGCAGAAAGAGGCGGAGCCTGCGGCCAAAGCTGATCGAGGAGCTGGCCGC |
| | | AACCCGGATCGTGGACGTGTCCACCGGAGATTCCCACTGTCTGGCCCTGTCCCACG |
| | | ATAACGAGGTGAGAGGACGGAAGAGAAGGTCTTATGCCTACGACAAAAGAGCCAA |
| | | CCCACAGGTGGGAGCCGCCTTCCCCCACATCAAGCACAACTACGAGTGTCTGGTGT |
| | | ATGTGCAGCTGCCATTCATGCGCGGCAGAAAGAGACGCAGCAGGCGCTATCCTTCT |
| | | ATGGCCCGCATCCACAGCATGACCATCGAGGCCCCCATCGCAAAGGACGGACTGC |
| | | GGCGCCTGAGCGGCAATGAGTACGTGTTTAGGGGCAGAAAGAGACGGTCCATCTC |
| | | CACAGCAGTGAGGCCCACCACACCCACAACCTGCAGCAGGAGGTGGTGGCGCCCA |
| | | TCCAGCGTGGGAGGCAGCGCCTCTGGCTCCAGGCCTCCA |

TABLE 7
Amino Acid Sequences

| SEQ ID | | |
|---|---|---|
| NO | Name | Amino Acid Insert Sequence |
| 43 | Patient | MDWTWILFLVAAATRVHSQSYLDSGIHSGATTTAPYLSGKGNPEEEDVDTSRGRKRRS |
| | PT4 | HLVSRKSISAALEHKVSALLPPAEQTGTWKLKTRGRKRRSDLRSRIEVLKRKVIEKLQHI |
| | | QLLQKNVRAQLVDRGRKRRSSKDLLRQLPASNFNSLRFLIVHLKRVVDHAEENRGRKR |
| | | RSTLLLDGNPFRVPQAAILMKGTAAILEYLRDRIPRGRKRRSSILESSSAFPDNAARREV |
| | | ESPPAVCPSDGCTWKRGRKRRSGQPYFEAFKKKMPIAFVAKGINKLLNKLFLINERGRK |
| | | RRSMEDLQDMFIVHTIEEIEGLIAAHDQFKSTLPDARGRKRRSMGGQTALNCGEELFKR |
| | | GVLKEYGVKVLGTSVESRGRKRRSAKGVNGSSQAPTTGKYCRLCDIQFNNLSNFITHR |
| | | GRKRRSHMKLKVQGMECLDGCYVHSSIVPTFHRRCTWLLRGRKRRSQASRGEVPSGA |
| | | YGYSYIPSGAYVYPPPVANGMYRGRKRRSYSSLRSIVVANYEESIKMPISEPAPGKKKS |
| | | QIQRGRKRRSDLQVSAKIGFLKHHENKTYLAVGSMKTLMLNVSRGRKRRSELILPSGA |
| | | RVGHRSLRRYYKQRFGLSRAVAVAKRGRKRRSMLGEQGFQSLMETVDTGIVTQLQEF |
| | | LQGFRGRKRRSRQIWWERLQGCQHIVEDWQKILMVRSLVVSPHERGRKRRSSTTGSNY |
| | | YVRILSTIDGELLKPNASVALHKHSNRGRKRRSANELCEVNRKGCPSADPCLPYFCVQG |
| | | CKLGEASRGRKRRSFEDCTSENTLIKYMTDRILLRESLREADLDHYSRGRKRRSWIRPL |
| | | ASGSYPSRKAGATLVVYKDLLVLFGGWTRGRKRRSSSDYSTSEMLVNVGNLPLDEFYP |
| | | AVSMVALMRIRGRKRRSCESPTAHCNVLNWEQVHRLDGILSETIPIHGRGRGRKRRSN |
| | | KQNKTPLDKSTSGVSEILLKTQMKMSLKCLAARGRKRRSYVCGGMFFFVSFEGKIVMH |
| | | KIIYMVLFLFCVALRGRKRRSLPTPGSWWEQLTQASRAYASGGTEGFPLSRWAPRGRK |
| | | RRSYSTLSLFDKYKGKSVDTIRSSVIPRHGLQSLGKRGRKRRSMNISLVWCLLVLSFAIR |
| | | RWWRGRKRRSRVELQQEVEKLAHENSSMRLELDALRSKYEALQRGRKRRSSPQREPR |
| | | GGQLRTPRMWPSCSRSLESLRVGAKPRGRKRRSFTLWNESTHYQILLTSFPHVENHSCF |
| | | EHMHHIPRGRKRRSDFVVEAIGTEVGTLGFSIERPSQAKIECDDKGDRGRKRRSNVIDS |
| | | MLFVFGYRAQKNKIRGRKRRSMMGSARVAELLLLHGAEPNCTDPATLTRPVHDARGR |
| | | KRRSPPGLDKRLLPETLGPCYSNSQPVWLCLTPRQPLRGRKRRSSFLLSPNNGNLEATS |
| | | NIEYAHVPHLSPAVIPTRRGRKRRSEEDEEDTYYTKDLPIHTCSYCGIHDPACVVYCNR |
| | | GRKRRSLISRRDAEVVLTSRELGSLALNQSTGLPTLTLPRGRKRRSDFSSNDSSTCTMGL |
| | | VLTLWSDTLIHLDGDGGFSRGRKRRSSSAAPSVMDLSSRASAALHPGIPSPNCMQSPIR |
| 44 | Patient | MDWTWILFLVAAATRVHSPDRKAAVSHWQQQSYLGSGIHSGATTTAPSLSGRGRKRR |
| | PT1 | SGQSPTLLTSACFFPSAAFTAEFLFSLGSLCQHLRGRKRRSRLASAPPQKQKQKLGERLF |
| | | PLIQAMHPTLAGKIRGRKRRSCGHLYAYDWISIPVVYTQVVTVAVYSFFLTCLVRGRK |
| | | RRSDCMSEDGYKLDRIKGEFEFHNVTFHYPSRPEVKRGRKRRSMANDSPAKSLVDIDL |
| | | PSLRDPAGIFELVEVVGNRGRKRRSWLGIRISESPEPGQRTFTTFKFCQLPFYKYSEYRG |
| | | RKRRSPPLARPILPRERGAMDRIVEYLVGDGPQNRYALRGRKRRSTCVSLDDGLYECSC |
| | | APAYSGKDCQKKDGPCVINRGRKRRSLHVGLCSGPCEMAEQRCCVDYAKRGTAGCK |
| | | KCKRGRKRRSSFESYLKTMPPYFLGPLKKAVRMMGAPNLIADSRGRKRRSPKFHSYVS |
| | | LPFGCTRAVVEYRLLQAAYLAKPGDRGRKRRSGEYVDTGKLIDKINLPNFLKVYLNHK |
| | | PPFGNTMRGRKRRSMCTNARRVRKRWLPKINSMLPEGVEMYRTVMGSRGRKRRSPD |
| | | GPLLPSPGLLRLPGGPRHAVSGVPEVYPPGPRGRKRRSVGTQATKYNSEVVDKMIEEFL |
| | | SSFEEKIENLTERGRKRRSTNLPIFKLKESCVRRRYSDFKWLKNELERDSKIRGRKRRSLI |
| | | RWCLALRPSDRPTFEEIQKHPWMQDVLLPQERGRKRRSKFECGWCSGERRCTLHQHC |
| | | TNPSSPWLDWSSHNRGRKRRSHDHTGFLTEYVATRWYSAPEIMLNSKGYTKSIDRGRK |
| | | RRSSSEATEVNPESLAKECLEKSLLLLKFLPTGISSRGRKRRSVKKDRSASHLDHKLEIR |
| | | QWRRSFLVSLFFCIPVRGRKRRSLFTFAVGVNICLGFTAYRIKRAEGWEEGPPTVLRGR |
| | | KRRSMSQAPGAQLSPPTVYHERQRLELCARGRKRRSMSSNSFPYIEQSGGGEATELGQE |
| | | ATRGRKRRSQPCRKMLPDRSRAARYRANQKLVEYIVKAKGAERGRKRRSPVFHGMLE |
| | | RAPAEPCYRAPMEKLFYLPHVCSYTRGRKRRSTYSCVAENILGTGQRGSGAELDVQYP |
| | | PKKVTTVRGRKRRSSPSATEDSLQPATDLLNRSELPQSQKAMQTKDARGRKRRSDLRK |
| | | VPGTDPAFEVFPLHDHQCVLRGRKRRSDALDFKKDKGAFYLVFIWTMTRGRKRRSVD |
| | | NYKQIKADLDQLRLPVEKSELWVEKSSNYENRGRKRRSNAPENGYMKCSSEGDNYGA |
| | | TCEFSCIGGYELQGRGRKRRSESGSSGKSSSSLVRGVHLSSSGPALLAGLVSLDRGRKR |
| | | RSWTEKRASYELEFAKSTTKIAEAGKVSIQQQSHMRGRKRRSKILARKKSKRSALENSE |
| | | EHSTKYSNSNNSAGSGRGRKRRSAAAAAALAAALLLLRREDPGLGAGPSMAETEALR |
| | | GRKRRSLPSFPPFGSMNSNAAGSVSTQANTVQSGQLGGQRGRKRRSSGVVHRLEVDED |
| | | FEEHNAAKVHLMVHNLVPPFLRGRKRRSLQIKQQELYKNFLRFQVEEKKQREEAERER |
| | | LRI |
| 45 | Patient | MDWTWILFLVAAATRVHSEQQSLSSDADTLSLTDSSVDPPIQDPRGRKRRSDQNTEDY |
| | PT6 | LFQVILEKQIRIPHSLSVKAASVLKSRGRKRRSLREDGREQEIRDGGSLRMIHFGLVRGR |
| | | KRRSLLQLWPVQRLTVKKSTDNLFREQKSSLNYLRQKRGRKRRSQWLRAVAVESIHRF |
| | | CMQPQLLRSFCQSYDMKQHRGRKRRSLEAIVQLWRIPSFVTELYVNYDCDYYCSNLFE |
| | | ERGRKRRSTPPGQSPGSAPIIINIHRAQLTNPEVKYNYTEDRGRKRRSPYATVAPSTLAH |
| | | PQAQVLARQQALQHAQTLAHARGRKRRSQRGLLQQIGDALSSQRGRVPPAAPPAQPR |
| | | VPVTRGRKRRSMFRSKRSGLVRRLWRSGVVPDREEGGSGGGGGGRGRKRRSLEIKVSP |
| | | PEGAETRKVREIGPLCGRRRPRVRKSRGRKRRSTPNFIIPAQRAEPMRIVRQPMPPPGDL |
| | | EPPFQPRGRKRRSGTGALGLLLLFLLGYGLRRRPQKAEEPAPQAGGRGRKRRSDSHVSE |
| | | VLQENYRLKNKLEGLISEKNELKMKSERGRKRRSVSAYQVQSPSWMQPQPCILQHPGA |
| | | VLTPSMEHTRGRKRRSGRESIVKKCIHKATRKYVAVKFVSKKMKKKEQARGRKRRSGI |
| | | LRDPGSEIEDRQYRIDLQSINIGTAQWHQLKRGRKRRSDGTQVLKIYVKQTESHYILVS |
| | | WKVNSNVMTSNLRGRKRRSPPGYIPDELHQVARNGLFTSINSEGEFIPESMDRGRKRRS |
| | | PKFRGVKMIPPGIHFLHYSSMDKANPKEVGPRMRGRKRRSNVSSAEGWHVNVTLSIRP |
| | | STGTGVMLALVSGNNRGRKRRSCQRSFRTFQALKKHLEASHLELSEADIQQLYGGRGR |
| | | KRRSDSVDFRLRVSEPKAVFAKEQLACREVQAEVGASRGRKRRSDIKDGRFPYGSTQD |
| | | YLKPIILIKLVQLGMAKDDRGRKRRSENYRNLAFLGIALSKPDLITFLEQGKEPWNMKQ |
| | | RGRKRRSWNSLGKMSREEAMSAYVTEMKLVAQKVIDTVPLRGRKRRSTFQCVVSPSD |
| | | VAVVWFWDGALLQPSEKFAISQSRGRKRRSHVDLFDLESGEYLCPLCKSLYNTVIPIIPL |
| | | QPQRGRKRRSSEIISKLYIPKRKIISPRSIKDVLPPVEEAVDR |
| 46 | Patient | MDWTWILFLVAAATRVHSPDRKAAVSHWQQQSYLGSGIHSGATTTAPSLSGRGRKRR |
| | PT2 | SGDNTGEQVAVKSPKPESGGNHIADLKKEIEILRRGRKRRSKDHGVNSFLVYMAFKDL |
| | | FQLTDCQIYEVLSVIRRGRKRRSNTDGKIEFISTMEGYTYPVYGVQWHPEKAPYEWRG |
| | | RKRRSNNSVLQKQQLELMDTVDNLVNLCTKEGVLLKGGRGRKRRSDSNREKDMGLF |
| | | EVFSQHLPTTEPVDSSVSSSISRGRKRRSLKAEVFTEILNYIYSSTVIVKRQETVTDLAAA |
| | | GRGRKRRSHPNNVVSIKYCSHSGLMFSVSTSYIKVWDIRDSRGRKRRSTKNKRRKEYF |
| | | LAVQIRSLEEKIKVKSTEVEILERGRKRRSLVKPQKENEQAEKKNISLAFFLYDLLSLMD |
| | | RGFRGRKRRSVDIVILGLSYANLQTGSGVLSDSLSGGMQLLPDRGRKRRSITKITRRRHE |
| | | NLPHGVASVKEWFNYVTATRNEERGRKRRSAVLKVGWLFPLSEVPNFTLLMDGCGC |
| | | WRLKEDQRGRKRRSLVAALLLGSVCCGSAQLLFNKTKSVEFTFRGRKRRSSHVFGTNT |
| | | SSLELFLMNGKIKGPCWLEVKSPQLRGRKRRSSPYDHTPGMAGSLGYHPYEAPLGSYP |
| | | YGDPAYRRGRKRRSMTGSSLSLAHLLIISGLLCYSARGRKRRSAEEGDELQQRCLDLER |
| | | HLMLLSEEKQSLAQENARGRKRRSTMYHGVPVVGIPLFGDHYETMTRVQAKGMGILL |
| | | RGRKRRSQPASHILNSEKFPFSKYEDCLEQQMPSLRESPMRGRKRRSVEKSPAKKKATK |
| | | KAVGAGAAKRKATGPPVSELIRGRKRRSLLAAGPSAAAAKPNIPKVLLPFTRATRVNFT |
| | | LERGRKRRSLAPQDMRVFYAHTARAQLPVWNNWTQRAARYECRGRKRRSPAPLRRP |
| | | LAVLHAPVPTTALARLSRPQRACPSSRGRKRRSKHWFVLTDSSLKYYRDSTAEGADEL |
| | | DGEIDLRSRGRKRRSVKFLRKPGGNLGKVYHPGSMLSLAPTKGLLNEPRGRKRRSDYQ |
| | | VYLNASKVPGFADDPTELSCRVVDTKSGEARGRKRRSQRDDPVLETSHQNFRRSRYQE |
| | | AASPREALIRLRRGRKRRSHLLNYVWPNVFETSPHVIEAVMGALEGLRVAIGRGRKRR |
| | | SSRTSMDDLKAFTSLSLYMKPLRGRKRRSCAAYGGYINCMAVLMESNADPNIQDKEG |
| | | RTALHRGRKRRSDNRLPPKKVPGFCSFRDGLSFLVHCCNVIITAQRGRKRRSPYSTAVS |
| | | TSGCGEHLVHTILARECSHALQAEDARGRKRRSGVLKQYQIKGLEWLVSCTTTTRGRK |
| | | RRSSKSPSKRSKSQDQAKKSKSPTLRRRSQEKIGKARGRKRRSFSLWKGFTPYYACLGP |
| | | HTVLTFIFLEQMNKAYKRGRKRRSDSFLVERGKLLHKRDNDKVDTQEENFLPKYQRV |
| | | RGRKRRSFRTYHQMVLHSRVHRRARREMDSDGDRAARARCRGRKRRSLEENINSALD |
| | | IKVLTQSSQSAVRGGPGRSTMPTRGRKRRSGASVNAPPDPCKQSPVRLAAGSGLACFLL |
| | | WQLQ |
| 47 | Patient | MDWTWILFLVAAATRVHSQSYLDSGIHSGATTTAPPLSGKGNPEEEDVDTSRGRKRRS |
| | PT3 | WMGDFVTAWMVTDMMLQAKPYPDWGKSARAFWKRGRKRRSPTGRPYNVDTESAQ |
| | | LYQGPHNTLFGYSVVLHSHRGRKRRSEKLAAEEQFQALVKQMYQTLQDKTNQIDLLQ |
| | | AERGRKRRSNEKMDSIVQCRALNIAVDLGQIQYLFSDKTGTLRGRKRRSEELPVVGQL |
| | | LRLLLQHTPLRTHMLTNAILVQQIRGRKRRSNLPLLQIELLRSAIKALRPGRILVYSTCTL |
| | | SKARGRKRRSGFQCAAPLLILTNKFCFLSTYVFVSTFLTGKELRGRKRRSVTAAFLVSF |
| | | MPYHIQRNIHLHFLHNETKPCDSVRGRKRRSARPELLQSHFIPTIGRLRKKAGKVVSEEE |
| | | QLRLRGRKRRSDRVDAQDETAAEFTLRVNLTAARLGQLEGLLQARGRKRRSLVQSRQ |
| | | NLVAEERLSSGILGTIGFGRKSPLSNRRGRKRRSMAFRTICVLLEYLFVLSVRGRKRRSF |
| | | EKGRIYTFIGEVVVSVNPYQLLNIYGRDTIEQRGRKRRSWPAVFARVSVFVDWIHKVM |
| | | RLGRGRKRRSVCVSSDTLDGNSSSAGYSCNSPAKVSTTTDSPVRGRKRRSVEDAVVFS |
| | | TELEASAISPRVVVPLAETHCEEQGRGRKRRSAAQPYSNLSNLDVLNQVVRERDTKLP |
| | | KPQLEQPRGRKRRSEVKRSQRLRRGPSSPRRPYQEMEYERRGGRGDRRGRKRRSASSL |
| | | NENVDHSALLKLDTSVSERVSRFDSKPAPRGRKRRSCHPGPRDLLLLHLLQRKDNIDRG |
| | | RKRRSDLLRSVLQQRLIALGGIIAARISVRGRKRRSYTMQKHLEMNPHFGSHQYQILPL |
| | | HSQIPREEQRRGRKRRSCSSSLMALQNAYQAVHSGQCPAAIVGGINVLLKRGRKRRSFR |
| | | LNIQGLERTLLGFVSKTLDSASAQFAASALVRGRKRRSINPQGLSDPSPMSDPVLTQDIS |
| | | PPAQGVDHRQVRGRKRRSLGPHEEDQVVCGFKKLSEWGLCFHPSTDTHKKL |
| 48 | Patient | MDWTWILFLVAAATRVHSQEKAFFAQLQLGEETGEFLPIQPAQHIQSETSGRGRKRRSS |
| | PT5 | YLDSGIHSGATTTAPFLSGKGNPEEEDVDTSQRGRKRRSGCCPPGHGSRSRPPAQPACS |
| | | RAEQDILFLLRSPRGRKRRSLVSLQPAQMHRFWRGLSLHLDSVDAVYERTSDHRGRKR |
| | | RSIQLPIIQLRKVWAEAVRYVSGLKEDYSRLFQGQRGRKRRSIFTGQANVVSEYLLAYL |
| | | FLSLVAFFRLIAFLNLRGRKRRSGRRCIRATPRACRARCATCCVRSSAATSPCCGCRGR |
| | | KRRSMAFALAIRDTATYARQLMFSTTLLIVFFTVWVFRGRKRRSWRPLLRGPRLSLHT |
| | | AAKAAATATETTCQDVAATRGRKRRSEALAENPGLVNKSCYEVGWLIKMTLSNPSEL |
| | | DERGRKRRSPSGSFSTPGSATYGRYKPSPERYTAAPHPLLSLRGRKRRSLQTRLQPEEST |
| | | ETLDSNYVVGHVLNSRKQKQLLRGRKRRSLEVSPFFKCHEVIGSSVLFIHDKKEQAKV |
| | | WMIDRGRKRRSVAQPKKKLGRRLVFHSNPPSGNKQQLPLVNETKRGRKRRSVHIRRK |
| | | HSHQYSYLCEVCKYYTVTKGDMERHCARGRKRRSCHVGSEETRARVPVHDLHVGITK |
| | | RLKTMEVLEGRGRKRRSVTDYTFEDCQLALAEGHLRLPADTCLLEFARLVRGRKRRSE |
| | | IHLEIFESASAGTRLLLDPATDPDININSIKDRGRKRRSVYVSTQFPKYIQDIVAATLKLP |
| | | ANKVMCHVRRVRGRKRRSLTQSKSSPKRGFFREETDRLIKNLLGKRISKLIRGRKRRSN |
| | | NMIHQVPIKSLPQEWLWCEMWCDDASKKRAKTRGRKRRSPENIEITVTLFKDPHAEEF |
| | | EGKEWTFVIENESPRGRKRRSAASRPRLDPWKLVSFSRTLTISPPSRPDTPESPRGRKRRS |
| | | SSKTTSVLQWAEKGYYTRSNNLVTLENGKQLTVRGRKRRSLDWQPRENPFQNLKVLS |
| | | VSDQQQNFLELWSEILRGRKRRSERWECTTYWYIFVFCSAHPAVTEMALFVTVFGLRG |
| | | RKRRSFYKKCAQLTVNLTRFPETVPGELVVPVAGSCVVRGRKRRSNNVDLILATPPFSR |
| | | LEKLYSGEVVRGRKRRSKTMKDFVRRRCWARKYKLVTSGPWLEVPPIALRRGRKRRS |
| | | ARRMAFLAKKGYRHDSSTAVVGSPRGHGQSRETRGRKRRSWRRAQKLHFLQRGGLK |
| | | QLSARVRPELKTKQQILRGRKRRSSGRLDQLLSLSMVWANHVFLGCSYNKDLLDKVM |
| | | RGRKRRSELGSLIRQLSLCSVKLYADPSVPDVVIDILQQIRGRKRRSPELSISSLTSPIPEN |
| | | STETEVALFRIRDRDSGERGRKRRSTSELLGEGAYAKVQGAMSLQNGKEYAVKIIEKQ |
| | | RGRKRRSDWEGNRAYSQYDKFHIGNEKQNYRLYLKGHTGTRGRKRRSLRPKLIEELA |
| | | ATRIVDVSTGDSHCLALSHDNEVRGRKRRSYAYDKRANPQVGAAFPHIKHNYECLVY |
| | | VQLPFMRGRKRRSRRYPSMARIHSMTIEAPIAKDGLRRLSGNEYVFRGRKRRSISTAVR |
| | | PTTPTTCSRRWWRPSSVGGSASGSRPP |

The disclosure relates to a nucleic acid molecule comprising a first, second and third nucleic acid sequence, wherein the first nucleic acid sequence is a first DNA backbone domain of the nucleic acid molecule, the second nucleic acid sequence is the second DNA backbone domain of the nucleic acid molecule and the third nucleic acid sequence is an expressible nucleic acid sequence; wherein the expressible nucleic acid sequence comprises a plurality of antigen expression domains, in 5′ to 3′ orientation. In some embodiments, the expressible nucleic acid sequence comprises a nucleic acid sequence encoding a linker at the 5′ end of the first antigen expression domain. In some embodiments, the expressible nucleic acid sequence encodes a linker between each of the antigen expression domains. In some embodiments, the expressible nucleic acid sequence encodes a leader sequence, a plurality of antigen expression domains, each antigen expression domain separated by a linker sequence. In some embodiments, there are at least 20 antigen expression domains. In some embodiments, there are at least 20, 25, 30, 35, 40, 45, 50, 55, 60, 65 or 70 or more antigen expression domains. In some embodiments, the nucleic acid molecule comprises Formula I, Ia, II, IIa, or IIIa. In some embodiments, the nucleic acid molecule comprises one or a plurality of regulatory sequences operably linked to the expressible nucleic acid sequence. In some embodiments, there are at least 20, 25, 30, 35, 40, 45, 50, 55, 60, 65 or 70 or more antigen expression domains that comprise at least about 70%, 80%, 90%, 95%

In some embodiments, the first DNA backbone domain comprises a nucleic acid sequence comprising at least about 70%, 75%, 80%, 85%, 90%, 91%, 92%, 93%, 94%, 95%, 96%, 97%, 98%, 99% or 100% sequence identity to SEQ ID NO:64 or a functional fragment that comprises at least about 70%, 75%, 80%, 85%, 90%, 91%, 92%, 93%, 94%, 95%, 96%, 97%, 98%, 99% sequence identity to SEQ ID NO:64. In some embodiments, the second DNA backbone domain comprises a nucleic acid sequence comprising at least about 70%, 75%, 80%, 85%, 90%, 91%, 92%, 93%, 94%, 95%, 96%, 97%, 98%, 99% or 100% sequence identity to SEQ ID NO:65 or a functional fragment that comprises at least about 70%, 75%, 80%, 85%, 90%, 91%, 92%, 93%, 94%, 95%, 96%, 97%, 98%, 99% sequence identity to SEQ ID NO:65.

The disclosure also relates to a nucleic acid molecule comprising a first nucleic acid sequence and a second nucleic acid sequence, wherein the first nucleic acid sequence is a DNA backbone domain of the nucleic acid molecule and the second nucleic acid sequence is an expressible nucleic acid sequence; wherein the expressible nucleic acid sequence comprises a plurality of antigen expression domains, in 5′ to 3′ orientation. In some embodiments, the expressible nucleic acid sequence comprises a nucleic acid sequence encoding a linker at the 5′ end of the first antigen expression domain. In some embodiments, the expressible nucleic acid sequence encodes a linker between each of the antigen expression domains. In some embodiments, the expressible nucleic acid sequence encodes a leader sequence, a plurality of antigen expression domains, each antigen expression domain separated by a linker sequence. In some embodiments, there are at least 20 antigen expression domains. In some embodiments, there are at least 20, 25, 30, 35, 40, 45, 50, 55, 60, 65 or 70 or more antigen expression domains. In some embodiments, the nucleic acid molecule comprises Formula I, Ia, II, IIa, or IIIa. In some embodiments, the nucleic acid molecule comprises one or a plurality of regulatory sequences operably linked to the expressible nucleic acid sequence. In some embodiments, the first DNA backbone domain comprises a nucleic acid sequence comprising at least about 70%, 75%, 80%, 85%, 90%, 91%, 92%, 93%, 94%, 95%, 96%, 97%, 98%, 99% or 100% sequence identity to SEQ ID NO:63 or a functional fragment that comprises at least about 70%, 75%, 80%, 85%, 90%, 91%, 92%, 93%, 94%, 95%, 96%, 97%, 98%, 99% sequence identity to SEQ ID NO:63. In some embodiments, the first DNA backbone domain comprises a nucleic acid sequence comprising at least about 70%, 75%, 80%, 85%, 90%, 91%, 92%, 93%, 94%, 95%, 96%, 97%, 98%, 99% or 100% sequence identity to SEQ ID NO:67 or a functional fragment that comprises at least about 70%, 75%, 80%, 85%, 90%, 91%, 92%, 93%, 94%, 95%, 96%, 97%, 98%, 99% sequence identity to SEQ ID NO:67.

TABLE 8
Plasmid Sequences

| SEQ ID | | |
|---|---|---|
| NO | Name | Full Plasmid Sequence |
| 49 | Patient | gctgcttcgcgatgtacgggccagatatacgcgttgacattgattattgactagttattaatagtaatca |
| | PT4 | attacggggtcattagttcatagcccatatatggagttccgcgttacataacttacggtaaatggcccgc |
| | | ctggctgaccgcccaacgacccccgcccattgacgtcaataatgacgtatgttcccatagtaacgccaat |
| | | agggactttccattgacgtcaatgggtggagtatttacggtaaactgcccacttggcagtacatcaagtg |
| | | tatcatatgccaagtacgccccctattgacgtcaatgacggtaaatggcccgcctggcattatgcccagt |
| | | acatgaccttatgggactttcctacttggcagtacatctacgtattagtcatcgctattaccatggtgat |
| | | gcggttttggcagtacatcaatgggcgtggatagcggtttgactcacggggatttccaagtctccacccc |
| | | attgacgtcaatgggagtttgttttggcaccaaaatcaacgggactttccaaaatgtcgtaacaactccg |
| | | ccccattgacgcaaatgggcggtaggcgtgtacggtgggaggtctatataagcagagctctctggctaac |
| | | tagagaacccactgcttactggcttatcgaaattaatacgactcactatagggagacccaagctggctag |
| | | cgtttaaacttaagcttggtaccgagctcGGATCCGCC |
| | | ACCATGGATTGGACATGGATTCTCTTTCTCGTTGCAGCAGCCACACGCGTTCACAGT |
| | | CAGTCTTACCTGGATTCTGGCATCCACAGCGGAGCCACCACCACCGCCCCTTATCTG |
| | | AGCGGCAAGGGAAACCCAGAGGAGGAGGATGTGGATACATCCAGAGGCCGCAAG |
| | | CGGAGATCTCACCTGGTGTCCAGAAAGTCCATCTCTGCCGCCCTGGAGCACAAGGT |
| | | GAGCGCCCTGCTGCCCCCCGCCGAGCAGACCGGAACATGGAAGCTGAAAACCAGA |
| | | GGCAGGAAGAGAAGATCCGACCTGCGGTCCCGCATCGAGGTGCTGAAGAGGAAGG |
| | | TGATCGAGAAGCTGCAGCACATCCAGCTGCTGCAGAAGAATGTGCGGGCACAGCT |
| | | GGTGGACCGGGGAAGGAAGCGGCGCAGCAGCAAGGACCTGCTGCGGCAGCTGCCT |
| | | GCCTCCAACTTTAACTCTCTGCGCTTCCTGATCGTGCACCTGAAAAGAGTGGTGGAT |
| | | CACGCCGAGGAGAATCGGGGCAGAAAGCGCCGCTCCACCCTGCTGCTGGATGGCA |
| | | ACCCTTTCAGAGTGCCCCAGGCCGCAATCCTGATGAAGGGCACCGCCGCAATCCTG |
| | | GAGTACCTGAGGGACAGAATCCCTAGGGGCAGGAAGCGGCGGTCCTCCATCCTGG |
| | | AGAGCAGCTCCGCCTTTCCCGATAATGCCGCCCGCAGGGAGGTGGAGAGCCCCCCA |
| | | GCAGTCTGTCCATCTGACGGCTGCACATGGAAGAGAGGACGCAAGCGGAGATCCG |
| | | GGCAGCCATACTTCGAGGCATTCAAGAAGAAGATGCCCATCGCATTCGTGGCAAAG |
| | | GGCATCAATAAGCTGCTGAACAAGCTGTTTCTGATCAACGAGAGGGGCCGGAAGC |
| | | GCAGGAGCATGGAGGATCTGCAGGACATGTTTATCGTGCACACAATCGAGGAGATC |
| | | GAGGGCCTGATTGCAGCCCACGATCAGTTCAAGAGCACCCTGCCAGACGCCAGAG |
| | | GCCGGAAGAGGCGCAGCATGGGCGGCCAGACCGCCCTGAATTGCGGCGAGGAGCT |
| | | GTTTAAGAGAGGCGTGCTGAAGGAGTACGGCGTGAAGGTGCTGGGCACCTCTGTGG |
| | | AGAGCCGCGGCAGGAAGCGGCGCTCCGCCAAGGGCGTGAACGGCTCTAGCCAGGC |
| | | CCCTACCACCGGAAAGTACTGTCGCCTGTGCGACATCCAGTTTAATAATCTGAGCA |
| | | ACTTTATCACCCACAGAGGACGGAAGCGGCGGAGCCACATGAAGCTGAAAGTGCA |
| | | GGGAATGGAGTGTCTGGATGGCTGTTATGTCCACAGCAGCATCGTGCCAACATTCC |
| | | ATAGAAGATGCACATGGCTGCTGCGCGGCAGAAAAAGGAGATCTCAGGCAAGCAG |
| | | AGGCGAGGTGCCAAGCGGCGCCTACGGCTACAGCTATATACCTTCCGGAGCCTATG |
| | | TGTACCCCCCACCTGTGGCCAACGGCATGTATAGGGGCAGGAAGAGAAGGAGCTA |
| | | TTCCAGCCTGAGATCTATCGTGGTGGCCAATTACGAGGAGTCCATCAAGATGCCAA |
| | | TCAGCGAGCCAGCCCCTGGCAAGAAGAAGTCCCAGATCCAGCGCGGCAGGAAGAG |
| | | GCGCTCTGACCTGCAGGTGAGCGCCAAGATCGGCTTCCTGAAGCACCACGAGAATA |
| | | AGACATACCTGGCCGTGGGCTCTATGAAAACCCTGATGCTGAACGTGAGCAGGGGC |
| | | AGAAAGCGCAGGTCCGAGCTGATCCTGCCTAGCGGCGCCAGGGTGGGCCACAGGA |
| | | GCCTGAGGAGATACTATAAGCAGAGGTTCGGCCTGTCTAGAGCCGTGGCCGTGGCC |
| | | AAGCGGGGACGCAAGAGGAGGTCTATGCTGGGCGAGCAGGGATTCCAGTCCCTGA |
| | | TGGAAACCGTGGATACAGGCATCGTGACACAGCTGCAGGAGTTTCTGCAGGGATTC |
| | | AGGGGCCGCAAGAGGAGGTCCCGCCAGATCTGGTGGGAGAGACTGCAGGGCTGCC |
| | | AGCACATCGTGGAGGATTGGCAGAAGATCCTGATGGTGCGCTCCCTGGTGGTGAGC |
| | | CCCCACGAGAGAGGACGGAAGCGCAGGTCCAGCACCACCGGATCTAACTACTACG |
| | | TGAGAATCCTGTCTACAATCGATGGCGAGCTGCTGAAGCCAAATGCCAGCGTGGCC |
| | | CTGCACAAGCACTCCAACAGGGGCCGCAAGCGCAGGAGCGCCAACGAGCTGTGCG |
| | | AGGTGAACAGAAAGGGCTGCCCAAGCGCCGATCCATGTCTGCCTTACTTCTGCGTG |
| | | CAGGGCTGTAAGCTGGGCGAGGCCTCCCGGGGACGCAAGCGGAGGTCTTTTGAGG |
| | | ATTGCACATCTGAGAACACCCTGATCAAGTATATGACCGATCGCATCCTGCTGAGG |
| | | GAGTCTCTGCGCGAGGCAGACCTGGACCACTATAGCCGGGGAAGGAAAAGGAGAA |
| | | GCTGGATCAGACCACTGGCATCCGGCAGCTACCCTAGCCGCAAGGCAGGAGCCAC |
| | | ACTGGTGGTGTACAAGGATCTGCTGGTGCTGTTCGGGGGCTGGACACGCGGACGCA |
| | | AGAGAAGGAGCTCCTCCGACTACTCCACATCCGAGATGCTGGTGAACGTGGGAAA |
| | | CCTGCCCCTGGACGAGTTTTATCCCGCCGTGAGCATGGTGGCCCTGATGCGCATCC |
| | | GGGGCCGCAAGAGAAGATCCTGCGAGAGCCCCACAGCACACTGTAATGTGCTGAA |
| | | TTGGGAGCAGGTGCACAGACTGGACGGCATCCTGTCTGAGACAATCCCAATCCACG |
| | | GAAGGGGAAGGGGGAGGAAGAGAAGGTCCAATAAGCAGAATAAGACCCCACTGG |
| | | ATAAGTCTACATCCGGGGTGTCTGAGATCCTGCTGAAAACCCAGATGAAAATGAGC |
| | | CTGAAGTGCCTGGCCGCCCGCGGACGGAAGAGAAGGTCTTACGTGTGCGGCGGCA |
| | | TGTTCTTCTTTGTGAGCTTCGAGGGGAAGATCGTGATGCACAAGATCATCTATATGG |
| | | TGCTGTTCCTGTTTTGTGTGGCACTGAGAGGCAGAAAGAGGAGGTCCCTGCCTACC |
| | | CCAGGGTCTTGGTGGGAGCAGCTGACCCAGGCATCTCGGGCCTACGCAAGCGGAG |
| | | GCACCGAGGGCTTTCCTCTGAGCCGGTGGGCCCCCAGGGGCAGAAAGAGAAGGTC |
| | | TTATTCCACCCTGTCCCTGTTTGACAAGTACAAGGGCAAGTCCGTGGATACCATCCG |
| | | GAGCTCCGTGATCCCCCGCCACGGCCTGCAGTCCCTGGGCAAAAGAGGCAGAAAG |
| | | AGAAGAAGCATGAACATCTCTCTGGTGTGGTGCCTGCTGGTGCTGTCCTTCGCCATC |
| | | AGGAGGTGGTGGCGCGGCAGGAAGCGCCGGAGCAGGGTGGAGCTGCAGCAGGAG |
| | | GTGGAGAAGCTGGCACACGAGAATAGCTCCATGCGGCTGGAGCTGGATGCCCTGA |
| | | GGTCCAAATATGAGGCACTGCAGAGGGGGAGAAAGAGAAGGTCCAGCCCTCAGAG |
| | | AGAGCCAAGAGGCGGCCAGCTGAGAACCCCAAGAATGTGGCCTTCTTGTTCCAGA |
| | | AGCCTGGAGTCTCTGAGAGTGGGAGCCAAGCCCCGGGGAAGGAAGAGACGCTCCT |
| | | TCACACTGTGGAACGAGAGCACCCACTATCAGATCCTGCTGACCTCTTTTCCCCACG |
| | | TGGAGAATCACTCTTGTTTTGAGCACATGCACCACATCCCAAGAGGCAGAAAGCGG |
| | | CGGTCTGATTTTGTGGTGGAGGCCATCGGAACCGAGGTGGGCACCCTGGGATTCTC |
| | | CATCGAAAGACCTTCCCAGGCCAAGATCGAGTGCGACGACAAGGGCGATCGGGGC |
| | | AGGAAGCGCAGATCCAACGTGATCGACAGCATGCTGTTCGTGTTCGGATATAGAGC |
| | | CCAGAAGAATAAGATCCGCGGAAGGAAGAGGCGGTCTATGATGGGCAGCGCAAGG |
| | | GTGGCAGAGCTGCTGCTGCTGCACGGAGCAGAGCCCAACTGTACCGACCCTGCAAC |
| | | ACTGACCAGACCAGTGCACGACGCAAGAGGAAGGAAGAGAAGAAGCCCCCCAGG |
| | | CCTGGACAAGAGACTGCTGCCAGAGACACTGGGCCCCTGCTACTCCAATTCTCAGC |
| | | CTGTGTGGCTGTGTCTGACACCACGGCAGCCACTGAGAGGCAGGAAAAGAAGATC |
| | | TTCCTTCCTGCTGAGCCCTAATAATGGCAACCTGGAGGCCACATCTAATATCGAGT |
| | | ACGCACACGTGCCCCACCTGTCTCCTGCCGTGATCCCTACACGGAGGGGCCGGAAG |
| | | AGGAGGTCTGAGGAGGATGAGGAGGACACCTATTATACAAAGGACCTGCCCATCC |
| | | ACACCTGCAGCTACTGCGGAATCCACGACCCAGCCTGCGTGGTGTATTGTAACCGC |
| | | GGCAGGAAGAGACGGAGCCTGATCTCTAGGAGGGATGCCGAGGTGGTGCTGACAA |
| | | GCAGGGAGCTGGGCAGCCTGGCCCTGAACCAGTCTACAGGCCTGCCTACCCTGACC |
| | | CTGCCAAGAGGCCGGAAGCGGAGGTCTGACTTCTCTTCTAACGACTCCTCCACATG |
| | | CACCATGGGCCTGGTGCTGACACTGTGGTCTGATACCCTGATCCACCTGGACGGCG |
| | | ATGGAGGCTTTAGCAGAGGCAGAAAAAGGAGGTCCTCCAGCGCAGCCCCATCCGT |
| | | GATGGACCTGAGCTCCCGGGCATCTGCCGCACTGCACCCAGGCATCCCCTCCCCTA |
| | | ATTGTATGCAGTCTCCCATCCGCTGATAACTCGAGtctagagggcccgtttaaacccgctgatcagcctc |
| | | gactgtgccttctagttgccagccatctgttgtttgcccctcccccgtgccttccttgaccctggaaggt |
| | | gccactcccactgtcctttcctaataaaatgaggaaattgcatcgcattgtctgagtaggtgtcattcta |
| | | ttctggggggtggggtggggcaggacagcaagggggaggattgggaagacaatagcaggcatgctgggga |
| | | tgcggtgggctctatggcttctactgggcggttttatggacagcaagcgaaccggaattgccagctgggg |
| | | cgccctctggtaaggttgggaagccctgcaaagtaaactggatggctttcttgccgccaaggatctgatg |
| | | gcgcaggggatcaagctctgatcaagagacaggatgaggatcgtttcgcatgattgaacaagatggattg |
| | | cacgcaggttctccggccgcttgggtggagaggctattcggctatgactgggcacaacagacaatcggct |
| | | gctctgatgccgccgtgttccggctgtcagcgcaggggcgcccggttctttttgtcaagaccgacctgtc |
| | | cggtgccctgaatgaactgcaagacgaggcagcgcggctatcgtggctggccacgacgggcgttccttgc |
| | | gcagctgtgctcgacgttgtcactgaagcgggaagggactggctgctattgggcgaagtgccggggcagg |
| | | atctcctgtcatctcaccttgctcctgccgagaaagtatccatcatggctgatgcaatgcggcggctgca |
| | | tacgcttgatccggctacctgcccattcgaccaccaagcgaaacatcgcatcgagcgagcacgtactcgg |
| | | atggaagccggtcttgtcgatcaggatgatctggacgaagagcatcaggggctcgcgccagccgaactgt |
| | | tcgccaggctcaaggcgagcatgcccgacggcgaggatctcgtcgtgacccatggcgatgcctgcttgcc |
| | | gaatatcatggtggaaaatggccgcttttctggattcatcgactgtggccggctgggtgtggcggaccgc |
| | | tatcaggacatagcgttggctacccgtgatattgctgaagagcttggcggcgaatgggctgaccgcttcc |
| | | tcgtgctttacggtatcgccgctcccgattcgcagcgcatcgccttctatcgccttcttgacgagttctt |
| | | ctgaattattaacgcttacaatttcctgatgcggtattttctccttacgcatctgtgcggtatttcacac |
| | | cgcatcaggtggcacttttcggggaaatgtgcgcggaacccctatttgtttatttttctaaatacattca |
| | | aatatgtatccgctcatgagacaataaccctgataaatgcttcaataatagcacgtgctaaaacttcatt |
| | | tttaatttaaaaggatctaggtgaagatcctttttgataatctcatgaccaaaatcccttaacgtgagtt |
| | | ttcgttccactgagcgtcagaccccgtagaaaagatcaaaggatcttcttgagatcctttttttctgcgc |
| | | gtaatctgctgcttgcaaacaaaaaaaccaccgctaccagcggtggtttgtttgccggatcaagagctac |
| | | caactctttttccgaaggtaactggcttcagcagagcgcagataccaaatactgttcttctagtgtagcc |
| | | gtagttaggccaccacttcaagaactctgtagcaccgcctacatacctcgctctgctaatcctgttacca |
| | | gtggctgctgccagtggcgataagtcgtgtcttaccgggttggactcaagacgatagttaccggataagg |
| | | cgcagcggtcgggctgaacggggggttcgtgcacacagcccagcttggagcgaacgacctacaccgaact |
| | | gagatacctacagcgtgagctatgagaaagcgccacgcttcccgaagggagaaaggcggacaggtatccg |
| | | gtaagcggcagggtcggaacaggagagcgcacgagggagcttccagggggaaacgcctggtatctttata |
| | | gtcctgtcgggtttcgccacctctgacttgagcgtcgatttttgtgatgctcgtcaggggggcggagcct |
| | | atggaaaaacgccagcaacgcggcctttttacggttcctggccttttgctggccttttgctcacatgttc |
| | | tt |
| 50 | Patient | gctgcttcgcgatgtacgggccagatatacgcgttgacattgattattgactagttattaatagtaatca |
| | PT1 | attacggggtcattagttcatagcccatatatggagttccgcgttacataacttacggtaaatggcccgc |
| | | ctggctgaccgcccaacgacccccgcccattgacgtcaataatgacgtatgttcccatagtaacgccaat |
| | | agggactttccattgacgtcaatgggtggagtatttacggtaaactgcccacttggcagtacatcaagtg |
| | | tatcatatgccaagtacgccccctattgacgtcaatgacggtaaatggcccgcctggcattatgcccagt |
| | | acatgaccttatgggactttcctacttggcagtacatctacgtattagtcatcgctattaccatggtgat |
| | | gcggttttggcagtacatcaatgggcgtggatagcggtttgactcacggggatttccaagtctccacccc |
| | | attgacgtcaatgggagtttgttttggcaccaaaatcaacgggactttccaaaatgtcgtaacaactccg |
| | | ccccattgacgcaaatgggcggtaggcgtgtacggtgggaggtctatataagcagagctctctggctaac |
| | | tagagaacccactgcttactggcttatcgaaattaatacgactcactatagggagacccaagctggctag |
| | | cgtttaaacttaagcttggtaccgagctcGGATCCGCC |
| | | ACCATGGATTGGACCTGGATTCTCTTTCTTGTTGCTGCTGCCACTCGCGTTCACAGT |
| | | CCTGACAGGAAGGCCGCCGTGTCCCACTGGCAGCAGCAGAGCTATCTGGGCAGCG |
| | | GCATCCACTCTGGCGCCACAACCACCGCACCATCTCTGTCTGGGAGAGGCAGGAAA |
| | | CGGAGAAGCGGCCAGTCTCCTACACTGCTGACATCTGCCTGTTTCTTCCCATCTGCA |
| | | GCCTTTACAGCCGAGTTTCTGTTTAGCCTGGGCTCCCTGTGTCAGCACCTGCGGGGC |
| | | CGGAAGAGAAGATCTCGCCTGGCCAGCGCACCTCCACAGAAACAGAAGCAGAAGC |
| | | TGGGCGAGAGGCTGTTCCCCCTGATCCAGGCAATGCACCCAACACTGGCCGGCAAA |
| | | ATCAGGGGCAGAAAGAGGAGGTCTTGCGGCCACCTGTATGCCTACGATTGGATCTC |
| | | TATCCCAGTGGTGTACACCCAGGTGGTGACCGTGGCCGTGTATAGCTTTTTTCTGAC |
| | | CTGCCTGGTGAGGGGCAGGAAGAGACGCAGCGACTGTATGTCCGAGGATGGATAT |
| | | AAGCTGGATAGGATCAAGGGAGAGTTCGAGTTCCACAATGTGACCTTTCACTATCC |
| | | CAGCCGCCCCGAGGTGAAGAGAGGAAGGAAGCGGAGGTCTATGGCCAACGATTCC |
| | | CCTGCCAAGTCCCTGGTGGACATCGACCTGCCTTCCCTGAGGGACCCAGCCGGCAT |
| | | CTTCGAGCTGGTGGAGGTGGTGGGAAATAGGGGCCGCAAGAGACGCAGCTGGCTG |
| | | GGCATCCGGATCTCTGAGTCTCCAGAGCCAGGGCAGCGGACATTCACAACCTTTAA |
| | | GTTTTGCCAGCTGCCATTCTACAAGTATTCCGAGTATAGAGGCAGAAAGCGGAGGA |
| | | GCCCCCCCCTGGCAAGACCAATCCTGCCAAGGGAGCGCGGCGCAATGGATAGAAT |
| | | CGTGGAATACCTGGTGGGAGACGGCCCTCAGAACAGATACGCACTGAGAGGCCGG |
| | | AAAAGGAGAAGCACCTGCGTGTCCCTGGATGACGGGCTGTACGAGTGTAGCTGTGC |
| | | CCCTGCCTATTCCGGAAAGGATTGTCAGAAGAAGGATGGACCTTGCGTGATCAACA |
| | | GGGGCAGGAAGCGCAGAAGCCTGCACGTGGGCCTGTGTTCCGGCCCCTGTGAGAT |
| | | GGCCGAGCAGCGGTGCTGCGTGGACTACGCCAAGAGGGGCACCGCCGGGTGTAAG |
| | | AAGTGTAAGAGGGGCCGGAAGCGGCGCTCCAGCTTCGAGAGCTACCTGAAAACAA |
| | | TGCCACCATACTTTCTGGGCCCACTGAAGAAGGCAGTGCGCATGATGGGAGCCCCA |
| | | AACCTGATCGCAGACAGCAGGGGCAGAAAGCGCCGCAGCCCTAAGTTCCACTCCT |
| | | ACGTGTCTCTGCCATTCGGCTGCACAAGGGCCGTGGTGGAGTACAGGCTGCTGCAG |
| | | GCCGCATACCTGGCCAAGCCCGGCGACAGAGGCCGGAAGCGCCGCTCCGGAGAGT |
| | | ACGTGGATACAGGCAAGCTGATCGATAAGATCAATCTGCCTAATTTCCTGAAGGTG |
| | | TATCTGAACCACAAGCCACCTTTTGGCAACACCATGCGGGGCAGGAAAAGAAGAT |
| | | CTATGTGCACCAACGCCAGAAGGGTGAGAAAGAGATGGCTGCCTAAGATCAACAG |
| | | CATGCTGCCCGAGGGAGTGGAGATGTACCGCACCGTGATGGGATCTAGAGGGAGA |
| | | AAGAGAAGAAGCCCAGACGGCCCACTGCTGCCATCTCCCGGCCTGCTGAGACTGCC |
| | | AGGCGGGCCTCGGCACGCCGTGTCTGGAGTGCCTGAGGTGTACCCCCCTGGCCCTA |
| | | GAGGCAGGAAGCGGCGGAGCGTGGGCACACAGGCCACCAAGTATAATAGCGAGGT |
| | | GGTGGACAAGATGATCGAGGAGTTTCTGTCCAGCTTTGAGGAGAAGATCGAGAATC |
| | | TGACAGAGAGGGGACGGAAGCGCCGGAGCACAAACCTGCCTATCTTTAAGCTGAA |
| | | GGAGAGCTGCGTGCGCAGGAGATACTCTGATTTCAAGTGGCTGAAGAATGAGCTGG |
| | | AGAGAGACTCTAAGATCAGAGGCAGGAAAAGGCGCTCCCTGATCAGATGGTGTCT |
| | | GGCACTGCGCCCAAGCGACAGGCCAACCTTCGAGGAGATCCAGAAGCACCCATGG |
| | | ATGCAGGACGTGCTGCTGCCCCAGGAGCGGGGCCGGAAGCGCAGATCCAAGTTTG |
| | | AATGCGGCTGGTGTAGCGGCGAGAGGAGGTGTACACTGCACCAGCACTGCACAAA |
| | | CCCAAGCTCCCCATGGCTGGATTGGTCCAGCCACAACAGGGGACGGAAGAGACGG |
| | | AGCCACGACCACACCGGCTTCCTGACCGAGTACGTGGCCACAAGGTGGTACAGCGC |
| | | ACCCGAGATCATGCTGAATTCCAAAGGCTACACAAAGTCTATCGATAGGGGAAGG |
| | | AAGCGCCGCAGCTCCTCCGAGGCAACCGAGGTGAATCCAGAGTCTCTGGCAAAGG |
| | | AGTGCCTGGAGAAATCTCTGCTGCTGCTGAAGTTCCTGCCCACCGGCATCAGCTCC |
| | | AGGGGAAGGAAGAGGAGAAGCGTGAAGAAGGATAGATCCGCCTCTCACCTGGACC |
| | | ACAAACTGGAGATCCGCCAGTGGAGGAGGTCCTTTCTGGTGTCCCTGTTTTTCTGCA |
| | | TCCCCGTGCGCGGACGCAAGCGCAGGAGCCTGTTTACATTCGCCGTGGGAGTGAAT |
| | | ATCTGCCTGGGCTTTACCGCCTACAGGATCAAGAGAGCAGAGGGATGGGAGGAGG |
| | | GCCCTCCCACCGTGCTGCGGGGCAGGAAGAGGAGATCCATGAGCCAGGCCCCTGG |
| | | CGCACAGCTGTCTCCCCCAACCGTGTACCACGAGAGACAGAGGCTGGAGCTGTGTG |
| | | CAAGGGGCAGAAAGCGGCGCTCCATGTCTTCTAACAGCTTTCCCTACATCGAGCAG |
| | | TCCGGAGGAGGCGAGGCCACCGAGCTGGGCCAGGAGGCCACCAGGGGAAGGAAA |
| | | AGAAGGTCCCAGCCTTGTCGCAAGATGCTGCCTGATCGGTCCAGAGCCGCCAGATA |
| | | CAGGGCCAATCAGAAGCTGGTGGAGTATATCGTGAAGGCAAAGGGAGCCGAGAGG |
| | | GGCCGCAAGCGGAGAAGCCCTGTCTTTCACGGCATGCTGGAGAGAGCACCAGCCG |
| | | AGCCTTGCTATCGGGCCCCAATGGAGAAGCTGTTTTATCTGCCACACGTGTGCTCTT |
| | | ATACCCGGGGCCGCAAGCGGCGCTCCACCTACTCTTGCGTGGCCGAGAATATCCTG |
| | | GGCACAGGACAGAGAGGCTCCGGCGCCGAGCTGGATGTGCAGTATCCCCCCAAGA |
| | | AGGTGACCACCGTGAGAGGCAGGAAGCGCCGCTCCAGCCCCAGCGCAACAGAGGA |
| | | TTCCCTGCAGCCTGCCACAGACCTGCTGAACAGGTCTGAGCTGCCCCAGAGCCAGA |
| | | AGGCAATGCAGACCAAGGATGCCCGCGGCAGAAAGAGAAGGTCTGACCTGAGAAA |
| | | GGTGCCCGGCACAGACCCCGCCTTCGAGGTGTTTCCCCTGCACGATCACCAGTGTG |
| | | TGCTGCGGGGCCGCAAGCGCCGGAGCGACGCACTGGATTTCAAGAAGGATAAGGG |
| | | CGCCTTCTATCTGGTGTTCATCTGGACCATGACCAGAGGCAGGAAGAGAAGATCCG |
| | | TGGATAACTACAAGCAGATCAAGGCCGACCTGGATCAGCTGAGGCTGCCTGTCGAG |
| | | AAGTCCGAGCTGTGGGTGGAGAAGAGCTCTAACTATGAGAATAGGGGCAGAAAGA |
| | | GAAGATCCAATGCCCCCGAGAATGGATATATGAAGTGCTCCAGCGAGGGCGACAA |
| | | CTACGGCGCCACCTGCGAGTTCTCTTGCATCGGAGGCTATGAGCTGCAGGGCCGGG |
| | | GCCGCAAGAGAAGATCCGAGAGCGGAAGCAGCGGCAAGAGCTCCTCTTCCCTGGT |
| | | GCGGGGAGTCCACCTGAGCAGCTCCGGACCCGCCCTGCTGGCCGGCCTGGTGTCCC |
| | | TGGACAGGGGCCGGAAGAGGAGATCTTGGACAGAGAAGCGGGCCTCTTATGAGCT |
| | | GGAGTTCGCCAAGTCCACAACAAAGATCGCCGAGGCCGGCAAGGTGTCCATCCAG |
| | | CAACAGTCTCACATGAGAGGCAGAAAGCGCAGGAGCAAGATCCTGGCAAGGAAGA |
| | | AGTCCAAACGGTCTGCACTGGAGAACTCCGAGGAGCACTCCACAAAGTACTCTAAT |
| | | TCCAACAACTCTGCCGGCTCCGGGAGAGGCAGAAAAAGAAGGTCCGCAGCAGCAG |
| | | CCGCCGCACTGGCCGCAGCCCTGCTGCTGCTGAGGCGCGAGGACCCAGGACTGGG |
| | | GGCCGGCCCCAGCATGGCAGAGACAGAGGCCCTGCGCGGCAGAAAGAGGAGATCT |
| | | CTGCCTTCTTTTCCTCCTTTCGGCTCTATGAACTCTAACGCCGCAGGCAGCGTGAGC |
| | | ACCCAGGCCAATACCGTGCAGAGCGGACAGCTGGGAGGACAGAGAGGCAGAAAG |
| | | AGACGGTCCAGCGGAGTGGTGCACCGGCTGGAGGTGGACGAGGACTTCGAGGAGC |
| | | ACAATGCCGCAAAGGTGCATCTGATGGTGCACAATCTGGTGCCACCATTCCTGAGG |
| | | GGCAGGAAGCGGAGGAGCCTGCAGATCAAGCAGCAGGAGCTGTATAAGAACTTCC |
| | | TGAGATTCCAGGTGGAGGAGAAGAAGCAGCGGGAGGAGGCCGAGCGCGAGCGCCT |
| | | GAGAATCTGATAACTCGAGtctagagggcccgtttaaacccgctgatcagcctcgactgtgccttctagt |
| | | tgccagccatctgttgtttgcccctcccccgtgccttccttgaccctggaaggtgccactcccactgtcc |
| | | tttcctaataaaatgaggaaattgcatcgcattgtctgagtaggtgtcattctattctggggggtggggt |
| | | ggggcaggacagcaagggggaggattgggaagacaatagcaggcatgctggggatgcggtgggctctatg |
| | | gcttctactgggcggttttatggacagcaagcgaaccggaattgccagctggggcgccctctggtaaggt |
| | | tgggaagccctgcaaagtaaactggatggctttcttgccgccaaggatctgatggcgcaggggatcaagc |
| | | tctgatcaagagacaggatgaggatcgtttcgcatgattgaacaagatggattgcacgcaggttctccgg |
| | | ccgcttgggtggagaggctattcggctatgactgggcacaacagacaatcggctgctctgatgccgccgt |
| | | gttccggctgtcagcgcaggggcgcccggttctttttgtcaagaccgacctgtccggtgccctgaatgaa |
| | | ctgcaagacgaggcagcgcggctatcgtggctggccacgacgggcgttccttgcgcagctgtgctcgacg |
| | | ttgtcactgaagcgggaagggactggctgctattgggcgaagtgccggggcaggatctcctgtcatctca |
| | | ccttgctcctgccgagaaagtatccatcatggctgatgcaatgcggcggctgcatacgcttgatccggct |
| | | acctgcccattcgaccaccaagcgaaacatcgcatcgagcgagcacgtactcggatggaagccggtcttg |
| | | tcgatcaggatgatctggacgaagagcatcaggggctcgcgccagccgaactgttcgccaggctcaaggc |
| | | gagcatgcccgacggcgaggatctcgtcgtgacccatggcgatgcctgcttgccgaatatcatggtggaa |
| | | aatggccgcttttctggattcatcgactgtggccggctgggtgtggcggaccgctatcaggacatagcgt |
| | | tggctacccgtgatattgctgaagagcttggcggcgaatgggctgaccgcttcctcgtgctttacggtat |
| | | cgccgctcccgattcgcagcgcatcgccttctatcgccttcttgacgagttcttctgaattattaacgct |
| | | tacaatttcctgatgcggtattttctccttacgcatctgtgcggtatttcacaccgcatcaggtggcact |
| | | tttcggggaaatgtgcgcggaacccctatttgtttatttttctaaatacattcaaatatgtatccgctca |
| | | tgagacaataaccctgataaatgcttcaataatagcacgtgctaaaacttcatttttaatttaaaaggat |
| | | ctaggtgaagatcctttttgataatctcatgaccaaaatcccttaacgtgagttttcgttccactgagcg |
| | | tcagaccccgtagaaaagatcaaaggatcttcttgagatcctttttttctgcgcgtaatctgctgcttgc |
| | | aaacaaaaaaaccaccgctaccagcggtggtttgtttgccggatcaagagctaccaactctttttccgaa |
| | | ggtaactggcttcagcagagcgcagataccaaatactgttcttctagtgtagccgtagttaggccaccac |
| | | ttcaagaactctgtagcaccgcctacatacctcgctctgctaatcctgttaccagtggctgctgccagtg |
| | | gcgataagtcgtgtcttaccgggttggactcaagacgatagttaccggataaggcgcagcggtcgggctg |
| | | aacggggggttcgtgcacacagcccagcttggagcgaacgacctacaccgaactgagatacctacagcgt |
| | | gagctatgagaaagcgccacgcttcccgaagggagaaaggcggacaggtatccggtaagcggcagggtcg |
| | | gaacaggagagcgcacgagggagcttccagggggaaacgcctggtatctttatagtcctgtcgggtttcg |
| | | ccacctctgacttgagcgtcgatttttgtgatgctcgtcaggggggcggagcctatggaaaaacgccagc |
| | | aacgcggcctttttacggttcctggccttttgctggccttttgctcacatgttctt |
| 51 | Patient | gctgcttcgcgatgtacgggccagatatacgcgttgacattgattattgactagttattaatagtaatca |
| | PT6 | attacggggtcattagttcatagcccatatatggagttccgcgttacataacttacggtaaatggcccgc |
| | | ctggctgaccgcccaacgacccccgcccattgacgtcaataatgacgtatgttcccatagtaacgccaat |
| | | agggactttccattgacgtcaatgggtggagtatttacggtaaactgcccacttggcagtacatcaagtg |
| | | tatcatatgccaagtacgccccctattgacgtcaatgacggtaaatggcccgcctggcattatgcccagt |
| | | acatgaccttatgggactttcctacttggcagtacatctacgtattagtcatcgctattaccatggtgat |
| | | gcggttttggcagtacatcaatgggcgtggatagcggtttgactcacggggatttccaagtctccacccc |
| | | attgacgtcaatgggagtttgttttggcaccaaaatcaacgggactttccaaaatgtcgtaacaactccg |
| | | ccccattgacgcaaatgggcggtaggcgtgtacggtgggaggtctatataagcagagctctctggctaac |
| | | tagagaacccactgcttactggcttatcgaaattaatacgactcactatagggagacccaagctggctag |
| | | cgtttaaacttaagcttggtaccgagctcGGATCCGCC |
| | | ACCATGGATTGGACATGGATTCTTTTTCTCGTTGCAGCAGCTACACGCGTTCATAGC |
| | | GAGCAGCAGTCTCTGTCTAGCGACGCCGATACCCTGTCTCTGACCGACTCTAGCGT |
| | | GGACCCACCTATCCAGGACCCAAGGGGCAGAAAAAGGAGATCTGACCAGAACACC |
| | | GAGGATTACCTGTTTCAGGTGATCCTGGAGAAGCAGATCAGAATCCCCCACAGCCT |
| | | GAGCGTGAAGGCCGCCTCCGTGCTGAAGTCTAGAGGCCGGAAGAGAAGGAGCCTG |
| | | AGAGAGGATGGCCGGGAGCAGGAGATCCGCGACGGCGGCTCCCTGCGGATGATCC |
| | | ACTTCGGCCTGGTGAGAGGCCGCAAGAGACGGAGCCTGCTGCAGCTGTGGCCAGT |
| | | GCAGCGGCTGACAGTGAAGAAGTCCACCGACAATCTGTTCAGAGAGCAGAAATCC |
| | | TCCCTGAACTACCTGCGCCAGAAGCGGGGCAGAAAGCGCCGGTCCCAGTGGCTGA |
| | | GGGCCGTGGCAGTGGAGAGCATCCACCGCTTCTGTATGCAGCCACAGCTGCTGAGA |
| | | TCCTTCTGTCAGAGCTATGATATGAAGCAGCACAGAGGAAGGAAGCGGAGGAGCC |
| | | TGGAGGCCATCGTGCAGCTGTGGCGCATCCCATCCTTCGTGACCGAGCTGTATGTG |
| | | AATTATGATTGTGATTATTATTGCTCCAATCTGTTCGAGGAGAGGGGGAGAAAGCG |
| | | CAGATCCACCCCTCCCGGCCAGTCCCCAGGCTCCGCCCCCATCATCATCAACATCC |
| | | ACAGAGCCCAGCTGACAAACCCAGAGGTGAAGTACAATTATACCGAGGACAGGGG |
| | | ACGGAAGAGACGCTCTCCTTATGCCACAGTGGCACCCTCCACCCTGGCCCACCCTC |
| | | AGGCCCAGGTGCTGGCCAGGCAGCAGGCCCTGCAGCACGCCCAGACCCTGGCCCA |
| | | CGCCAGAGGCAGAAAACGGAGAAGCCAGAGGGGCCTGCTGCAGCAGATCGGCGAC |
| | | GCACTGTCCAGCCAGAGGGGACGGGTCCCCCCAGCCGCACCCCCTGCACAGCCCAG |
| | | GGTGCCAGTGACCAGAGGCAGAAAGCGCAGGTCCATGTTTAGGTCTAAGAGATCC |
| | | GGCCTGGTGAGGAGGCTGTGGCGGTCCGGCGTGGTGCCCGATCGCGAGGAGGGAG |
| | | GAAGCGGCGGCGGCGGGGGAGGCCGCGGCAGAAAGAGGCGGTCCCTGGAGATCA |
| | | AGGTGAGCCCACCCGAGGGCGCAGAGACACGCAAGGTGAGAGAGATCGGCCCTCT |
| | | GTGCGGACGCAGAAGGCCACGGGTGAGAAAGTCCAGGGGCAGGAAGAGAAGGTC |
| | | TACCCCTAACTTTATCATCCCAGCCCAGAGAGCCGAGCCTATGAGGATCGTGCGCC |
| | | AGCCTATGCCACCCCCCGGGGACCTGGAGCCTCCTTTCCAGCCTAGAGGCAGAAAG |
| | | CGGCGCTCTGGCACAGGCGCCCTGGGCCTGCTGCTGCTGTTCCTGCTGGGCTATGG |
| | | ACTGAGGAGAAGGCCTCAGAAGGCCGAGGAGCCTGCACCTCAGGCAGGCGGCCGG |
| | | GGACGGAAGAGAAGATCTGACAGCCACGTGTCCGAGGTGCTGCAGGAGAACTATC |
| | | GGCTGAAAAACAAGCTGGAGGGACTGATCAGCGAGAAGAATGAGCTGAAGATGAA |
| | | GAGCGAGAGGGGACGGAAGCGGAGATCTGTGTCTGCATACCAAGTGCAGAGCCCA |
| | | AGCTGGATGCAGCCTCAGCCATGTATCCTGCAGCACCCTGGCGCCGTGCTGACCCC |
| | | TTCTATGGAGCACACCAGGGGCCGGAAACGGAGGAGCGGCAGGTTCAGCATCGTG |
| | | AAGAAGTGCATCCACAAGGCAACCAGGAAGTACGTGGCCGTGAAGTTCGTGTCCA |
| | | AGAAGATGAAAAAGAAGGAGCAGGCCCGCGGACGCAAGCGCCGGTCTGGCATCCT |
| | | GCGCGACCCAGGCTCTGAGATCGAGGACAGACAGTATAGAATCGATCTGCAGTCC |
| | | ATCAATATCGGAACCGCCCAGTGGCACCAGCTGAAGAGAGGAAGGAAGAGAAGGT |
| | | CCGACGGAACACAGGTGCTGAAGATCTACGTGAAGCAGACAGAGTCCCACTACAT |
| | | CCTGGTGTCTTGGAAGGTGAATTCTAACGTGATGACATCTAATCTGAGAGGCCGCA |
| | | AGCGCCGGAGCCCCCCAGGATACATCCCCGATGAGCTGCACCAGGTGGCCCGGAA |
| | | CGGCCTGTTTACATCTATCAACTCTGAGGGCGAGTTCATCCCCGAGAGCATGGATA |
| | | GGGGCAGAAAGAGACGGAGCCCAAAGTTTCGGGGAGTGAAAATGATCCCTCCAGG |
| | | CATTCACTTCCTGCACTATAGCAGCATGGACAAAGCCAATCCTAAGGAGGTGGGCC |
| | | CACGCATGAGAGGCAGAAAGAGAAGATCCAATGTGAGCAGCGCCGAGGGATGGCA |
| | | CGTGAACGTGACCCTGTCTATCAGGCCCTCCACAGGCACCGGCGTGATGCTGGCAC |
| | | TGGTGAGCGGAAATAACAGGGGCCGCAAGAGGAGATCTTGTCAGAGGTCTTTTAG |
| | | GACCTTTCAGGCCCTGAAGAAGCACCTGGAGGCCTCCCACCTGGAGCTGAGCGAGG |
| | | CCGATATCCAGCAGCTGTACGGCGGCCGCGGCAGGAAGCGCAGATCCGACTCCGT |
| | | GGACTTTCGCCTGAGGGTGAGCGAGCCCAAGGCAGTGTTCGCCAAGGAGCAGCTG |
| | | GCATGCAGGGAGGTGCAGGCAGAGGTGGGCGCCTCTAGAGGCAGGAAGAGGCGCA |
| | | GCGATATCAAGGATGGAAGATTTCCTTACGGATCTACCCAGGATTACCTGAAGCCA |
| | | ATCATCCTGATCAAGCTGGTGCAGCTGGGCATGGCAAAGGATGACAGGGGCAGAA |
| | | AGAGGCGCTCCGAGAACTACCGGAACCTGGCCTTTCTGGGGATCGCACTGTCTAAG |
| | | CCAGACCTGATCACATTCCTGGAGCAGGGCAAGGAGCCCTGGAATATGAAGCAGA |
| | | GGGGCAGGAAGCGGAGATCCTGGAATAGCCTGGGCAAAATGTCTCGCGAGGAGGC |
| | | CATGAGCGCATACGTGACCGAGATGAAGCTGGTGGCCCAGAAGGTGATCGATACC |
| | | GTGCCCCTGAGAGGGCGGAAGCGCAGGTCCACATTTCAGTGCGTGGTGTCCCCCTC |
| | | CGACGTGGCAGTGGTGTGGTTCTGGGATGGAGCCCTGCTGCAGCCCAGCGAAAAGT |
| | | TTGCCATCTCCCAGAGCAGGGGCAGAAAGCGGAGGTCCCACGTGGATCTGTTCGAC |
| | | CTGGAGAGCGGAGAGTATCTGTGCCCACTGTGCAAGAGCCTGTACAACACAGTGAT |
| | | CCCAATCATCCCACTGCAGCCTCAGCGCGGCAGGAAGAGGAGATCTAGCGAGATC |
| | | ATCAGCAAGCTGTACATCCCCAAGAGGAAGATCATCTCTCCAAGAAGCATCAAGG |
| | | ACGTGCTGCCACCAGTGGAGGAGGCCGTGGATAGGTGATAACTCGAGtctagagggcccgt |
| | | ttaaacccgctgatcagcctcgactgtgccttctagttgccagccatctgttgtttgcccctcccccgtg |
| | | ccttccttgaccctggaaggtgccactcccactgtcctttcctaataaaatgaggaaattgcatcgcatt |
| | | gtctgagtaggtgtcattctattctggggggtggggggggcaggacagcaagggggaggattgggaagac |
| | | aatagcaggcatgctggggatgcggtgggctctatggcttctactgggcggttttatggacagcaagcga |
| | | accggaattgccagctggggcgccctctggtaaggttgggaagccctgcaaagtaaactggatggctttc |
| | | ttgccgccaaggatctgatggcgcaggggatcaagctctgatcaagagacaggatgaggatcgtttcgca |
| | | tgattgaacaagatggattgcacgcaggttctccggccgcttgggtggagaggctattcggctatgactg |
| | | ggcacaacagacaatcggctgctctgatgccgccgtgttccggctgtcagcgcaggggcgcccggttctt |
| | | tttgtcaagaccgacctgtccggtgccctgaatgaactgcaagacgaggcagcgcggctatcgtggctgg |
| | | ccacgacgggcgttccttgcgcagctgtgctcgacgttgtcactgaagcgggaagggactggctgctatt |
| | | gggcgaagtgccggggcaggatctcctgtcatctcaccttgctcctgccgagaaagtatccatcatggct |
| | | gatgcaatgcggcggctgcatacgcttgatccggctacctgcccattcgaccaccaagcgaaacatcgca |
| | | tcgagcgagcacgtactcggatggaagccggtcttgtcgatcaggatgatctggacgaagagcatcaggg |
| | | gctcgcgccagccgaactgttcgccaggctcaaggcgagcatgcccgacggcgaggatctcgtcgtgacc |
| | | catggcgatgcctgcttgccgaatatcatggtggaaaatggccgcttttctggattcatcgactgtggcc |
| | | ggctgggtgtggcggaccgctatcaggacatagcgttggctacccgtgatattgctgaagagcttggggc |
| | | gaatgggctgaccgcttcctcgtgctttacggtatcgccgctcccgattcgcagcgcatcgccttctatc |
| | | gccttcttgacgagttcttctgaattattaacgcttacaatttcctgatgcggtattttctccttacgca |
| | | tctgtgcggtatttcacaccgcatcaggtggcacttttcggggaaatgtgcgcggaacccctatttgttt |
| | | atttttctaaatacattcaaatatgtatccgctcatgagacaataaccctgataaatgcttcaataatag |
| | | cacgtgctaaaacttcatttttaatttaaaaggatctaggtgaagatcctttttgataatctcatgacca |
| | | aaatcccttaacgtgagttttcgttccactgagcgtcagaccccgtagaaaagatcaaaggatcttcttg |
| | | agatcctttttttctgcgcgtaatctgctgcttgcaaacaaaaaaaccaccgctaccagcggtggtttgt |
| | | ttgccggatcaagagctaccaactctttttccgaaggtaactggcttcagcagagcgcagataccaaata |
| | | ctgttcttctagtgtagccgtagttaggccaccacttcaagaactctgtagcaccgcctacatacctcgc |
| | | tctgctaatcctgttaccagtggctgctgccagtggcgataagtcgtgtcttaccgggttggactcaaga |
| | | cgatagttaccggataaggcgcagcggtcgggctgaacggggggttcgtgcacacagcccagcttggagc |
| | | gaacgacctacaccgaactgagatacctacagcgtgagctatgagaaagcgccacgcttcccgaagggag |
| | | aaaggcggacaggtatccggtaagcggcagggtcggaacaggagagcgcacgagggagcttccaggggga |
| | | aacgcctggtatctttatagtcctgtcgggtttcgccacctctgacttgagcgtcgatttttgtgatgct |
| | | cgtcaggggggcggagcctatggaaaaacgccagcaacgcggcctttttacggttcctggccttttgctg |
| | | gccttttgctcacatgttctt |
| 52 | Patient | gctgcttcgcgatgtacgggccagatatacgcgttgacattgattattgactagttattaatagtaatca |
| | PT2 | attacggggtcattagttcatagcccatatatggagttccgcgttacataacttacggtaaatggcccgc |
| | | ctggctgaccgcccaacgacccccgcccattgacgtcaataatgacgtatgttcccatagtaacgccaat |
| | | agggactttccattgacgtcaatgggtggagtatttacggtaaactgcccacttggcagtacatcaagtg |
| | | tatcatatgccaagtacgccccctattgacgtcaatgacggtaaatggcccgcctggcattatgcccagt |
| | | acatgaccttatgggactttcctacttggcagtacatctacgtattagtcatcgctattaccatggtgat |
| | | gcggttttggcagtacatcaatgggcgtggatagcggtttgactcacggggatttccaagtctccacccc |
| | | attgacgtcaatgggagtttgttttggcaccaaaatcaacgggactttccaaaatgtcgtaacaactccg |
| | | ccccattgacgcaaatgggcggtaggcgtgtacggtgggaggtctatataagcagagctctctggctaac |
| | | tagagaacccactgcttactggcttatcgaaattaatacgactcactatagggagacccaagctggctag |
| | | cgtttaaacttaagcttggtaccgagctcGGATCCGCC |
| | | ACCATGGATTGGACTTGGATTCTCTTTCTCGTTGCCGCAGCTACACGCGTTCACTCA |
| | | CCTGATCGGAAAGCCGCAGTGAGCCACTGGCAGCAGCAGTCCTACCTGGGCAGCG |
| | | GAATCCACTCTGGAGCCACAACAACCGCACCATCTCTGTCTGGCCGGGGCAGGAAG |
| | | AGGAGGTCCGGCGATAACACAGGCGAGCAGGTGGCCGTGAAGTCCCCTAAGCCTG |
| | | AGAGCGGAGGCAATCACATCGCCGATCTGAAGAAGGAGATCGAGATCCTGAGGAG |
| | | AGGGAGAAAGCGGAGATCTAAGGATCACGGCGTGAACTCCTTTCTGGTGTACATGG |
| | | CCTTCAAGGACCTGTTTCAGCTGACAGATTGTCAGATCTACGAGGTGCTGTCCGTCA |
| | | TCCGGAGAGGCAGAAAGCGGAGGTCTAACACAGACGGCAAAATCGAGTTCATCTC |
| | | TACCATGGAGGGCTACACCTACCCAGTGTATGGCGTGCAGTGGCATCCTGAGAAGG |
| | | CACCCTACGAGTGGAGGGGCCGCAAGAGGAGATCCAATAATAGCGTGCTGCAGAA |
| | | GCAGCAGCTGGAGCTGATGGATACCGTGGACAATCTGGTGAACCTGTGTACCAAGG |
| | | AGGGCGTGCTGCTGAAGGGAGGCCGCGGCAGAAAGAGACGCAGCGACTCCAACCG |
| | | GGAGAAGGACATGGGACTGTTCGAGGTGTTCTCTCAGCACCTGCCCACCACCGAGC |
| | | CAGTGGACAGCTCCGTGAGCTCTAGCATCAGCAGAGGCCGCAAGCGGCGCAGCCT |
| | | GAAGGCCGAGGTGTTCACAGAGATCCTGAATTACATCTATTCCAGCACAGTGATCG |
| | | TGAAGAGGCAGGAAACCGTGACCGACCTGGCCGCCGCCGGCAGAGGCAGAAAGAG |
| | | ACGGTCCCACCCAAATAACGTGGTGTCCATCAAGTACTGCAGCCACTCCGGGCTGA |
| | | TGTTTAGCGTGTCCACCTCCTACATCAAGGTGTGGGATATCCGCGACAGCAGGGGC |
| | | AGAAAAAGAAGGTCCACAAAGAACAAGCGGAGAAAGGAGTATTTTCTGGCAGTGC |
| | | AGATCCGGAGCCTGGAGGAAAAGATCAAGGTGAAGTCTACAGAGGTGGAGATCCT |
| | | GGAGAGAGGAAGGAAGCGCCGGTCCCTGGTGAAGCCACAGAAGGAGAATGAGCA |
| | | GGCCGAGAAGAAGAACATCTCTCTGGCCTTCTTTCTGTATGACCTGCTGTCCCTGAT |
| | | GGACAGAGGCTTTAGAGGACGGAAGAGGCGGTCTGTGGATATCGTGATCCTGGGC |
| | | CTGAGCTACGCCAACCTGCAGACCGGCAGCGGAGTGCTGAGCGATTCTCTGAGCGG |
| | | CGGAATGCAGCTGCTGCCAGATAGGGGCAGAAAGCGGCGCTCTATCACCAAAATC |
| | | ACCAGGAGACGCCACGAGAATCTGCCCCACGGCGTGGCAAGCGTGAAAGAGTGGT |
| | | TCAATTACGTCACCGCAACACGCAACGAGGAGAGAGGACGCAAGAGGCGGAGCGC |
| | | CGTGCTGAAGGTGGGATGGCTGTTTCCTCTGTCCGAGGTGCCTAACTTTACACTGCT |
| | | GATGGACGGCTGTGGCTGCTGGAGGCTGAAGGAGGATCAGCGGGGCCGCAAGAGA |
| | | AGATCTCTGGTGGCCGCCCTGCTGCTGGGCTCCGTGTGCTGCGGCTCCGCCCAGCTG |
| | | CTGTTCAATAAGACCAAAAGCGTGGAGTTCACCTTCAGGGGCCGGAAGAGGCGCTC |
| | | CAGCCACGTGTTTGGAACCAACACCAGCTCTCTGGAGCTGTTCCTGATGAACGGCA |
| | | AGATCAAGGGACCCTGTTGGCTGGAGGTGAAGTCTCCACAGCTGAGAGGAAGGAA |
| | | GAGAAGGAGCTCTCCCTACGATCACACACCAGGGATGGCAGGCTCTCTGGGCTACC |
| | | ACCCCTACGAGGCCCCCCTGGGAAGCTATCCATATGGCGACCCAGCCTATAGGAGG |
| | | GGCAGAAAGCGCAGATCCATGACAGGGTCCTCCCTGTCCCTGGCCCACCTGCTGAT |
| | | CATCAGCGGCCTGCTGTGCTATTCCGCAAGGGGCAGGAAGCGGCGGTCCGCCGAG |
| | | GAGGGAGACGAGCTGCAGCAGAGATGTCTGGACCTGGAGAGACACCTGATGCTGC |
| | | TGAGCGAGGAGAAGCAGTCCCTGGCCCAGGAGAATGCCAGGGGCAGGAAGCGCAG |
| | | ATCTACCATGTATCACGGAGTGCCTGTGGTGGGCATCCCTCTGTTCGGCGACCACTA |
| | | CGAGACAATGACCCGCGTGCAGGCCAAGGGAATGGGCATCCTGCTGAGGGGCAGG |
| | | AAGAGACGCTCCCAGCCTGCATCTCACATCCTGAATAGCGAGAAATTCCCTTTCTC |
| | | CAAGTACGAGGACTGCCTGGAGCAGCAGATGCCCAGCCTGCGCGAGTCTCCAATGC |
| | | GCGGCAGGAAGAGACGGTCTGTGGAGAAGAGCCCCGCAAAGAAGAAGGCCACCA |
| | | AGAAGGCCGTGGGCGCCGGAGCAGCCAAGAGAAAGGCCACCGGCCCACCCGTGAG |
| | | CGAGCTGATCCGCGGCAGAAAACGCAGATCCCTGCTGGCAGCCGGCCCATCCGCCG |
| | | CCGCCGCCAAGCCAAATATCCCTAAGGTGCTGCTGCCATTTACAAGGGCAACAAGA |
| | | GTGAACTTTACCCTGGAGAGGGGCAGAAAAAGGAGGAGCCTGGCACCCCAGGACA |
| | | TGAGAGTGTTTTACGCACACACCGCCAGGGCCCAGCTGCCCGTGTGGAACAATTGG |
| | | ACCCAGAGAGCAGCCCGCTATGAGTGCCGGGGCCGCAAGCGGAGGTCCCCTGCAC |
| | | CTCTGAGACGCCCCCTGGCAGTGCTGCACGCCCCAGTGCCAACAACCGCACTGGCA |
| | | AGACTGTCCAGGCCTCAGCGGGCATGTCCATCCTCTAGGGGCAGAAAGAGGCGCA |
| | | GCAAGCACTGGTTCGTGCTGACAGATTCCTCCCTGAAGTACTACAGAGACTCTACA |
| | | GCAGAGGGCGCCGACGAGCTGGATGGAGAGATCGATCTGCGCAGCAGAGGCAGGA |
| | | AGAGGCGGAGCGTGAAGTTTCTGAGGAAGCCAGGCGGAAATCTGGGAAAGGTGTA |
| | | TCACCCAGGCTCCATGCTGTCCCTGGCACCTACAAAGGGCCTGCTGAATGAGCCTA |
| | | GAGGCCGGAAGCGCAGGTCTGATTATCAGGTGTACCTGAACGCCAGCAAGGTGCC |
| | | CGGATTCGCCGATGATCCTACAGAGCTGTCCTGTCGGGTGGTGGATACAAAGAGCG |
| | | GCGAGGCACGCGGGAGGAAGAGGAGGAGCCAGCGGGATGATCCTGTGCTGGAGAC |
| | | CAGCCACCAGAACTTTAGGCGCTCCAGATATCAGGAGGCCGCATCTCCAAGGGAG |
| | | GCCCTGATCCGCCTGCGCCGGGGCAGGAAGCGGAGATCTCACCTGCTGAATTATGT |
| | | GTGGCCAAACGTGTTCGAGACCTCTCCACACGTGATCGAGGCAGTGATGGGCGCCC |
| | | TGGAGGGCCTGAGAGTGGCCATCGGCAGAGGCAGGAAACGCAGGAGCAGCAGGA |
| | | CATCTATGGACGATCTGAAGGCCTTCACATCCCTGAGCCTGTATATGAAGCCTCTGC |
| | | GCGGACGCAAGCGGAGATCTTGCGCCGCCTATGGAGGCTATATCAACTGCATGGCC |
| | | GTGCTGATGGAGAGCAACGCCGATCCTAATATCCAAGACAAGGAGGGACGCACAG |
| | | CCCTGCACAGGGGCAGAAAGAGAAGGTCTGATAACCGGCTGCCCCCCAAGAAGGT |
| | | GCCCGGATTTTGCAGCTTCCGCGACGGCCTGAGCTTCCTGGTGCACTGCTGTAACGT |
| | | GATCATCACAGCCCAGAGAGGCCGGAAGAGGAGATCCCCATACTCTACCGCCGTGT |
| | | CTACCTCTGGCTGCGGAGAGCACCTGGTGCACACCATCCTGGCAAGAGAGTGTTCC |
| | | CACGCCCTGCAGGCAGAGGACGCCAGAGGCCGCAAGAGAAGGTCCGGCGTGCTGA |
| | | AACAGTATCAGATCAAGGGCCTGGAGTGGCTGGTGTCCTGTACCACAACAACCCGG |
| | | GGCAGAAAGAGGAGGTCCTCTAAGTCTCCCTCCAAGAGAAGCAAGTCCCAGGACC |
| | | AGGCAAAGAAGAGCAAGAGCCCAACACTGAGAAGAAGGAGCCAGGAGAAGATCG |
| | | GCAAGGCAAGGGGAAGGAAGCGGAGGAGCTTTTCTCTGTGGAAGGGCTTTACACC |
| | | CTACTACGCCTGCCTGGGACCTCACACCGTGCTGACCTTCATCTTTCTGGAGCAGAT |
| | | GAACAAGGCATATAAGAGAGGCCGCAAGAGGCGCAGCGATTCTTTCCTGGTGGAG |
| | | CGGGGAAAGCTGCTGCACAAGCGGGATAACGACAAAGTGGATACCCAGGAGGAGA |
| | | ATTTCCTGCCAAAGTACCAGCGGGTGAGGGGCAGGAAAAGAAGGAGCTTTCGGAC |
| | | CTACCACCAGATGGTGCTGCACTCTAGGGTGCACCGCAGAGCCCGGAGAGAGATG |
| | | GACTCTGACGGAGACAGGGCCGCCAGAGCCCGCTGTAGAGGCAGGAAGCGCAGGA |
| | | GCCTGGAGGAGAATATCAATTCTGCCCTGGACATCAAGGTGCTGACCCAGAGCTCC |
| | | CAGTCTGCCGTGAGGGGAGGCCCCGGGAGATCCACCATGCCCACCAGAGGGCGGA |
| | | AGAGACGGTCCGGCGCCAGCGTGAACGCCCCCCCAGACCCTTGCAAGCAGAGCCC |
| | | AGTGAGACTGGCAGCCGGCTCTGGCCTGGCCTGCTTCCTGCTGTGGCAGCTGCAGT |
| | | GATAACTCGAGtctagagggcccgtttaaacccgctgatcagcctcgactgtgccttctagttgccagcc |
| | | atctgttgtttgcccctcccccgtgccttccttgaccctggaaggtgccactcccactgtcctttcctaa |
| | | taaaatgaggaaattgcatcgcattgtctgagtaggtgtcattctattctggggggtggggtggggcagg |
| | | acagcaagggggaggattgggaagacaatagcaggcatgctggggatgcggtgggctctatggcttctac |
| | | tgggcggttttatggacagcaagcgaaccggaattgccagctggggcgccctctggtaaggttgggaagc |
| | | cctgcaaagtaaactggatggctttcttgccgccaaggatctgatggcgcaggggatcaagctctgatca |
| | | agagacaggatgaggatcgtttcgcatgattgaacaagatggattgcacgcaggttctccggccgcttgg |
| | | gtggagaggctattcggctatgactgggcacaacagacaatcggctgctctgatgccgccgtgttccggc |
| | | tgtcagcgcaggggcgcccggttctttttgtcaagaccgacctgtccggtgccctgaatgaactgcaaga |
| | | cgaggcagcgcggctatcgtggctggccacgacgggcgttccttgcgcagctgtgctcgacgttgtcact |
| | | gaagcgggaagggactggctgctattgggcgaagtgccggggcaggatctcctgtcatctcaccttgctc |
| | | ctgccgagaaagtatccatcatggctgatgcaatgcggcggctgcatacgcttgatccggctacctgccc |
| | | attcgaccaccaagcgaaacatcgcatcgagcgagcacgtactcggatggaagccggtcttgtcgatcag |
| | | gatgatctggacgaagagcatcaggggctcgcgccagccgaactgttcgccaggctcaaggcgagcatgc |
| | | ccgacggcgaggatctcgtcgtgacccatggcgatgcctgcttgccgaatatcatggtggaaaatggccg |
| | | cttttctggattcatcgactgtggccggctgggtgtggcggaccgctatcaggacatagcgttggctacc |
| | | cgtgatattgctgaagagcttggcggcgaatgggctgaccgcttcctcgtgctttacggtatcgccgctc |
| | | ccgattcgcagcgcatcgccttctatcgccttcttgacgagttcttctgaattattaacgcttacaattt |
| | | cctgatgcggtattttctccttacgcatctgtgcggtatttcacaccgcatcaggtggcacttttcgggg |
| | | aaatgtgcgcggaacccctatttgtttatttttctaaatacattcaaatatgtatccgctcatgagacaa |
| | | taaccctgataaatgcttcaataatagcacgtgctaaaacttcatttttaatttaaaaggatctaggtga |
| | | agatcctttttgataatctcatgaccaaaatcccttaacgtgagttttcgttccactgagcgtcagaccc |
| | | cgtagaaaagatcaaaggatcttcttgagatcctttttttctgcgcgtaatctgctgcttgcaaacaaaa |
| | | aaaccaccgctaccagcggtggtttgtttgccggatcaagagctaccaactctttttccgaaggtaactg |
| | | gcttcagcagagcgcagataccaaatactgttcttctagtgtagccgtagttaggccaccacttcaagaa |
| | | ctctgtagcaccgcctacatacctcgctctgctaatcctgttaccagtggctgctgccagtggcgataag |
| | | tcgtgtcttaccgggttggactcaagacgatagttaccggataaggcgcagcggtcgggctgaacggggg |
| | | gttcgtgcacacagcccagcttggagcgaacgacctacaccgaactgagatacctacagcgtgagctatg |
| | | agaaagcgccacgcttcccgaagggagaaaggcggacaggtatccggtaagcggcagggtcggaacagga |
| | | gagcgcacgagggagcttccagggggaaacgcctggtatctttatagtcctgtcgggtttcgccacctct |
| | | gacttgagcgtcgatttttgtgatgctcgtcaggggggcggagcctatggaaaaacgccagcaacgcggc |
| | | ctttttacggttcctggccttttgctggccttttgctcacatgttctt |
| 53 | Patient | gctgcttcgcgatgtacgggccagatatacgcgttgacattgattattgactagttattaatagtaatca |
| | PT3 | attacggggtcattagttcatagcccatatatggagttccgcgttacataacttacggtaaatggcccgc |
| | | ctggctgaccgcccaacgacccccgcccattgacgtcaataatgacgtatgttcccatagtaacgccaat |
| | | agggactttccattgacgtcaatgggtggagtatttacggtaaactgcccacttggcagtacatcaagtg |
| | | tatcatatgccaagtacgccccctattgacgtcaatgacggtaaatggcccgcctggcattatgcccagt |
| | | acatgaccttatgggactttcctacttggcagtacatctacgtattagtcatcgctattaccatggtgat |
| | | gcggttttggcagtacatcaatgggcgtggatagcggtttgactcacggggatttccaagtctccacccc |
| | | attgacgtcaatgggagtttgttttggcaccaaaatcaacgggactttccaaaatgtcgtaacaactccg |
| | | ccccattgacgcaaatgggcggtaggcgtgtacggtgggaggtctatataagcagagctctctggctaac |
| | | tagagaacccactgcttactggcttatcgaaattaatacgactcactatagggagacccaagctggctag |
| | | cgtttaaacttaagcttggtaccgagctcGGATCCGCC |
| | | ACCATGGATTGGACATGGATTCTCTTTCTCGTTGCTGCAGCTACTCGCGTTCATAGC |
| | | CAGTCCTATCTGGACAGCGGCATCCACTCCGGCGCAACAACCACAGCACCTCCACT |
| | | GTCTGGAAAGGGAAATCCTGAGGAGGAGGATGTGGATACATCCCGCGGCAGGAAG |
| | | AGACGGTCTTGGATGGGCGACTTTGTGACAGCCTGGATGGTGACAGATATGATGCT |
| | | GCAGGCAAAGCCCTACCCAGACTGGGGGAAGTCTGCCAGGGCCTTCTGGAAAAGG |
| | | GGCAGGAAGAGACGCTCCCCCACCGGCAGACCATACAATGTGGATACCGAGTCTG |
| | | CACAGCTGTACCAGGGCCCCCACAATACCCTGTTTGGCTACTCCGTGGTGCTGCACT |
| | | CCCACCGGGGCAGAAAGAGGCGCAGCGAGAAGCTGGCAGCCGAGGAGCAGTTTCA |
| | | GGCCCTGGTGAAGCAGATGTATCAGACACTGCAGGACAAGACCAACCAGATCGAC |
| | | CTGCTGCAAGCCGAGAGGGGCCGCAAGAGAAGATCCAACGAGAAGATGGATTCCA |
| | | TCGTGCAGTGCCGCGCACTGAACATCGCAGTGGACCTGGGCCAGATCCAGTACCTG |
| | | TTTTCTGACAAGACAGGCACCCTGAGGGGCAGAAAGCGGCGCAGCGAGGAGCTGC |
| | | CCGTGGTGGGACAGCTGCTGAGACTGCTGCTGCAGCACACCCCTCTGAGAACACAC |
| | | ATGCTGACAAACGCCATCCTGGTGCAGCAGATCAGGGGGAGGAAGAGGAGGTCTA |
| | | ACCTGCCCCTGCTGCAGATCGAGCTGCTGCGGTCTGCAATCAAGGCCCTGAGGCCT |
| | | GGCCGCATCCTGGTGTACAGCACATGCACCCTGAGCAAGGCAAGAGGCCGGAAGC |
| | | GGAGAAGCGGATTTCAGTGTGCCGCCCCACTGCTGATCCTGACCAACAAATTCTGC |
| | | TTCCTGAGCACCTACGTCTTTGTGAGCACCTTCCTGACCGGCAAGGAGCTGAGAGG |
| | | CAGGAAACGCCGCTCCGTGACCGCAGCCTTCCTGGTGAGCTTTATGCCATACCACA |
| | | TCCAGCGCAATATCCACCTGCACTTTCTGCACAATGAGACAAAGCCATGTGATTCT |
| | | GTGAGGGGACGGAAGAGAAGGTCTGCCAGGCCCGAGCTGCTGCAGAGCCACTTCA |
| | | TCCCTACCATCGGCCGGCTGCGGAAGAAGGCCGGCAAGGTGGTGAGCGAGGAGGA |
| | | GCAGCTGAGGCTGAGAGGCAGAAAGCGCAGATCTGACCGGGTGGATGCCCAGGAC |
| | | GAAACCGCCGCCGAGTTCACCCTGCGGGTGAACCTGACCGCCGCCAGACTGGGCCA |
| | | GCTGGAGGGACTGCTGCAGGCCAGGGGCCGCAAGCGCCGGTCCCTGGTGCAGTCT |
| | | AGACAGAACCTGGTGGCCGAGGAGCGCCTGTCCAGCGGCATCCTGGGCACAATCG |
| | | GATTCGGACGCAAGTCCCCACTGTCCAACAGAAGAGGCAGGAAGAGAAGGAGCAT |
| | | GGCCTTTAGAACCATCTGCGTGCTGCTGGAGTATCTGTTCGTGCTGTCCGTGAGGGG |
| | | CAGGAAACGGAGATCCTTTGAGAAAGGACGCATCTATACATTTATCGGAGAGGTGG |
| | | TGGTGAGCGTGAACCCATATCAGCTGCTGAACATCTACGGCAGGGATACAATCGAG |
| | | CAGAGAGGAAGGAAGCGGAGAAGCTGGCCCGCCGTGTTCGCAAGAGTGAGCGTGT |
| | | TCGTGGATTGGATACACAAGGTGATGAGGCTGGGCCGGGGACGGAAGAGGAGGTC |
| | | TGTGTGCGTGAGCTCCGACACCCTGGATGGAAATTCCAGCAGCGCCGGCTACTCTT |
| | | GCAACAGCCCCGCAAAGGTGTCCACCACCACAGATTCTCCAGTCAGAGGCAGGAA |
| | | GAGGAGAAGCGTGGAGGACGCCGTGGTGTTCTCTACAGAGCTGGAGGCCAGCGCC |
| | | ATCTCCCCAAGGGTGGTGGTGCCTCTGGCCGAGACACACTGTGAGGAGCAGGGCA |
| | | GAGGCAGAAAGCGGAGATCTGCCGCCCAGCCCTACTCTAATCTGTCCAATCTGGAC |
| | | GTGCTGAATCAGGTGGTGAGGGAGAGAGACACAAAGCTGCCAAAGCCTCAGCTGG |
| | | AGCAGCCTCGGGGCAGAAAGAGAAGAAGCGAGGTGAAGAGAAGCCAGCGCCTGC |
| | | GCCGCGGACCTTCCTCCCCTAGGCGCCCATATCAGGAGATGGAGTATGAGAGGAGA |
| | | GGAGGAAGAGGCGACAGAAGAGGCAGAAAGAGAAGGAGCGCATCTAGCCTGAAT |
| | | GAGAACGTGGACCACAGCGCACTGCTGAAGCTGGATACCAGCGTGTCTGAGAGGG |
| | | TGTCTAGGTTCGATTCCAAGCCTGCCCCTAGAGGGCGGAAGCGGAGGTCTTGTCAC |
| | | CCAGGCCCAAGGGACCTGCTGCTGCTGCACCTGCTGCAGAGAAAGGACAATATCG |
| | | ACCGCGGCCGCAAGCGGAGAAGCGACCTGCTGCGCAGCGTCCTGCAGCAGAGACT |
| | | GATCGCCCTGGGCGGCATCATCGCCGCACGCATCAGCGTGAGAGGCAGAAAACGC |
| | | CGGTCCTATACCATGCAGAAACACCTGGAGATGAACCCCCACTTCGGCTCCCACCA |
| | | GTATCAGATCCTGCCCCTGCACTCTCAGATCCCAAGGGAGGAGCAGCGGAGAGGCC |
| | | GCAAGCGGAGGTCCTGTTCTTCTTCTCTGATGGCCCTGCAGAATGCCTACCAGGCA |
| | | GTGCACAGCGGCCAGTGCCCCGCCGCCATCGTGGGCGGCATCAACGTGCTGCTGAA |
| | | GAGGGGGAGGAAGCGGAGGAGCTTCAGGCTGAACATCCAGGGCCTGGAGCGGACC |
| | | CTGCTGGGCTTCGTGTCTAAGACCCTGGACTCCGCATCCGCCCAGTTTGCAGCATCC |
| | | GCCCTGGTGAGGGGAAGGAAGAGGCGGAGCATCAATCCTCAGGGACTGTCCGATC |
| | | CCTCCCCAATGAGCGATCCAGTGCTGACCCAGGATATCAGCCCTCCTGCCCAGGGA |
| | | GTGGACCACCGGCAGGTGAGAGGCAGGAAGCGCCGCTCCCTGGGCCCACACGAGG |
| | | AGGATCAGGTGGTGTGCGGATTCAAGAAGCTGTCTGAGTGGGGCCTGTGTTTTCAC |
| | | CCCTCCACAGATACCCACAAGAAGCTGTGATAACTCGAGtctagagggcccgtttaaacccgctgat |
| | | cagcctcgactgtgccttctagttgccagccatctgttgtttgcccctcccccgtgccttccttgaccct |
| | | ggaaggtgccactcccactgtcctttcctaataaaatgaggaaattgcatcgcattgtctgagtaggtgt |
| | | cattctattctggggggtggggtggggcaggacagcaagggggaggattgggaagacaatagcaggcatg |
| | | ctggggatgcggtgggctctatggcttctactgggcggttttatggacagcaagcgaaccggaattgcca |
| | | gctggggcgccctctggtaaggttgggaagccctgcaaagtaaactggatggctttcttgccgccaagga |
| | | tctgatggcgcaggggatcaagctctgatcaagagacaggatgaggatcgtttcgcatgattgaacaaga |
| | | tggattgcacgcaggttctccggccgcttgggtggagaggctattcggctatgactgggcacaacagaca |
| | | atcggctgctctgatgccgccgtgttccggctgtcagcgcaggggcgcccggttctttttgtcaagaccg |
| | | acctgtccggtgccctgaatgaactgcaagacgaggcagcgcggctatcgtggctggccacgacgggcgt |
| | | tccttgcgcagctgtgctcgacgttgtcactgaagcgggaagggactggctgctattgggcgaagtgccg |
| | | gggcaggatctcctgtcatctcaccttgctcctgccgagaaagtatccatcatggctgatgcaatgcggc |
| | | ggctgcatacgcttgatccggctacctgcccattcgaccaccaagcgaaacatcgcatcgagcgagcacg |
| | | tactcggatggaagccggtcttgtcgatcaggatgatctggacgaagagcatcaggggctcgcgccagcc |
| | | gaactgttcgccaggctcaaggcgagcatgcccgacggcgaggatctcgtcgtgacccatggcgatgcct |
| | | gcttgccgaatatcatggtggaaaatggccgcttttctggattcatcgactgtggccggctgggtgtggc |
| | | ggaccgctatcaggacatagcgttggctacccgtgatattgctgaagagcttggcggcgaatgggctgac |
| | | cgcttcctcgtgctttacggtatcgccgctcccgattcgcagcgcatcgccttctatcgccttcttgacg |
| | | agttcttctgaattattaacgcttacaatttcctgatgcggtattttctccttacgcatctgtgcggtat |
| | | ttcacaccgcatcaggtggcacttttcggggaaatgtgcgcggaacccctatttgtttatttttctaaat |
| | | acattcaaatatgtatccgctcatgagacaataaccctgataaatgcttcaataatagcacgtgctaaaa |
| | | cttcatttttaatttaaaaggatctaggtgaagatcctttttgataatctcatgaccaaaatcccttaac |
| | | gtgagttttcgttccactgagcgtcagaccccgtagaaaagatcaaaggatcttcttgagatcctttttt |
| | | tctgcgcgtaatctgctgcttgcaaacaaaaaaaccaccgctaccagcggtggtttgtttgccggatcaa |
| | | gagctaccaactctttttccgaaggtaactggcttcagcagagcgcagataccaaatactgttcttctag |
| | | tgtagccgtagttaggccaccacttcaagaactctgtagcaccgcctacatacctcgctctgctaatcct |
| | | gttaccagtggctgctgccagtggcgataagtcgtgtcttaccgggttggactcaagacgatagttaccg |
| | | gataaggcgcagcggtcgggctgaacggggggttcgtgcacacagcccagcttggagcgaacgacctaca |
| | | ccgaactgagatacctacagcgtgagctatgagaaagcgccacgcttcccgaagggagaaaggcggacag |
| | | gtatccggtaagcggcagggtcggaacaggagagcgcacgagggagcttccagggggaaacgcctggtat |
| | | ctttatagtcctgtcgggtttcgccacctctgacttgagcgtcgatttttgtgatgctcgtcaggggggc |
| | | ggagcctatggaaaaacgccagcaacgcggcctttttacggttcctggccttttgctggccttttgctca |
| | | catgttctt |
| 54 | Patient | gctgcttcgcgatgtacgggccagatatacgcgttgacattgattattgactagttattaatagtaatca |
| | PT5 | attacggggtcattagttcatagcccatatatggagttccgcgttacataacttacggtaaatggcccgc |
| | | ctggctgaccgcccaacgacccccgcccattgacgtcaataatgacgtatgttcccatagtaacgccaat |
| | | agggactttccattgacgtcaatgggtggagtatttacggtaaactgcccacttggcagtacatcaagtg |
| | | tatcatatgccaagtacgccccctattgacgtcaatgacggtaaatggcccgcctggcattatgcccagt |
| | | acatgaccttatgggactttcctacttggcagtacatctacgtattagtcatcgctattaccatggtgat |
| | | gcggttttggcagtacatcaatgggcgtggatagcggtttgactcacggggatttccaagtctccacccc |
| | | attgacgtcaatgggagtttgttttggcaccaaaatcaacgggactttccaaaatgtcgtaacaactccg |
| | | ccccattgacgcaaatgggcggtaggcgtgtacggtgggaggtctatataagcagagctctctggctaac |
| | | tagagaacccactgcttactggcttatcgaaattaatacgactcactatagggagacccaagctggctag |
| | | cgtttaaacttaagcttggtaccgagctcGGATCCGCC |
| | | ACCATGGATTGGACTTGGATTCTTTTTCTCGTTGCTGCTGCTACTCGCGTTCACAGTC |
| | | AGGAGAAGGCCTTCTTCGCACAGCTGCAGCTGGGAGAGGAAACCGGAGAGTTCCT |
| | | GCCTATCCAGCCCGCCCAGCACATCCAGTCTGAGACCTCCGGAAGGGGCAGAAAA |
| | | AGGCGGAGCTCTTATCTGGATTCCGGAATTCACTCCGGCGCCACCACCACAGCACC |
| | | CTTCCTGTCTGGAAAAGGAAATCCTGAGGAGGAGGACGTGGACACAAGCCAGCGC |
| | | GGAAGGAAGCGGCGCTCCGGATGTTGCCCACCCGGCCACGGCTCTAGGAGCAGGC |
| | | CCCCAGCCCAGCCCGCCTGCTCTAGAGCAGAGCAGGATATCCTGTTTCTGCTGAGA |
| | | AGCCCAAGGGGCAGAAAGAGGAGGTCCCTGGTGTCCCTGCAGCCAGCCCAGATGC |
| | | ACAGGTTCTGGAGAGGGCTGTCCCTGCACCTGGACTCCGTGGATGCCGTGTATGAG |
| | | AGAACATCTGACCACAGAGGCAGGAAAAGGAGAAGCATCCAGCTGCCAATCATCC |
| | | AGCTGAGAAAAGTGTGGGCCGAGGCCGTGAGATACGTGTCTGGCCTGAAGGAGGA |
| | | TTACTCCAGACTGTTTCAGGGCCAACGCGGAAGGAAGAGGAGATCTATCTTTACCG |
| | | GCCAGGCAAATGTGGTGAGCGAGTATCTGCTGGCCTACCTGTTCCTGTCCCTGGTG |
| | | GCATTTTTCAGACTGATCGCCTTTCTGAATCTGCGGGGCAGGAAGAGACGGTCTGG |
| | | CAGGAGATGTATCCGGGCCACCCCCAGAGCCTGCAGAGCAAGATGTGCCACATGCT |
| | | GCGTGCGGTCCAGCGCCGCCACAAGCCCCTGTTGCGGATGCAGAGGCCGCAAGAG |
| | | GAGATCCATGGCCTTCGCCCTGGCCATCAGAGACACCGCAACCTATGCCAGACAGC |
| | | TGATGTTTTCTACCACCCTGCTGATCGTGTTCTTCACCGTCTGGGTGTTTCGCGGAC |
| | | GGAAGCGCAGGTCTTGGCGGCCCCTGCTGAGAGGCCCCAGGCTGAGCCTGCACACC |
| | | GCCGCCAAGGCCGCCGCCACCGCCACAGAGACAACATGCCAGGATGTGGCAGCAA |
| | | CCCGGGGCCGCAAGCGCCGCAGCGAGGCCCTGGCCGAGAACCCAGGCCTGGTGAA |
| | | CAAGAGCTGTTATGAGGTGGGCTGGCTGATCAAGATGACACTGTCTAATCCTAGCG |
| | | AGCTGGACGAGCGCGGCCGCAAGAGACGCTCCCCTTCCGGCTCCTTCAGCACCCCA |
| | | GGCTCTGCAACATACGGCAGGTATAAGCCCAGCCCTGAGCGCTACACCGCAGCCCC |
| | | ACACCCACTGCTGTCCCTGAGGGGCAGAAAGCGCAGGAGCCTGCAGACAAGACTG |
| | | CAGCCTGAGGAGAGCACAGAGACCCTGGACTCCAACTACGTGGTGGGCCACGTGC |
| | | TGAATAGCCGGAAGCAGAAGCAGCTGCTGCGGGGCAGAAAAAGAAGGAGCCTGG |
| | | AGGTGTCTCCCTTTTTCAAGTGTCACGAGGTGATCGGATCTTCTGTGCTGTTCATCC |
| | | ACGATAAGAAGGAGCAGGCAAAGGTGTGGATGATCGACAGAGGAAGGAAGCGGC |
| | | GGAGCGTGGCACAGCCAAAGAAGAAGCTGGGCAGGAGACTGGTGTTTCACAGCAA |
| | | CCCCCCCAGCGGAAATAAGCAGCAGCTGCCCCTGGTGAATGAGACAAAGAGGGGC |
| | | CGGAAGAGGAGGTCTGTGCACATCAGGCGCAAGCACAGCCACCAGTACAGCTATC |
| | | TGTGCGAGGTGTGTAAGTATTATACAGTGACAAAGGGAGACATGGAGAGACACTG |
| | | CGCCAGAGGACGGAAGCGCCGGTCTTGTCACGTCGGCTCTGAGGAGACAAGGGCA |
| | | AGAGTGCCAGTGCACGACCTGCACGTGGGCATCACCAAGAGACTGAAAACAATGG |
| | | AGGTGCTGGAGGGCAGAGGCAGAAAGAGAAGGTCTGTGACCGACTACACCTTTGA |
| | | GGATTGTCAGCTGGCACTGGCCGAGGGACACCTGCGCCTGCCTGCCGACACATGTC |
| | | TGCTGGAGTTCGCAAGACTGGTGAGGGGCCGGAAGCGCCGGAGCGAGATCCACCT |
| | | GGAGATCTTTGAGAGCGCCAGCGCCGGCACCCGCCTGCTGCTGGACCCCGCCACCG |
| | | ACCCAGATATCAACATCAACTCTATCAAGGATCGGGGAAGGAAGCGGAGATCCGT |
| | | GTATGTGAGCACCCAGTTCCCTAAGTACATCCAGGATATCGTGGCAGCCACACTGA |
| | | AGCTGCCTGCCAACAAGGTGATGTGTCACGTGAGACGGGTGAGGGGGAGAAAGAG |
| | | AAGGAGCCTGACCCAGAGCAAGAGCTCCCCAAAGAGGGGCTTCTTTAGGGAGGAG |
| | | ACAGACAGGCTGATCAAAAACCTGCTGGGCAAGAGGATCTCCAAGCTGATCAGAG |
| | | GCCGGAAGAGGCGCAGCAACAACATGATCCACCAGGTGCCAATCAAGTCCCTGCC |
| | | TCAGGAGTGGCTGTGGTGCGAGATGTGGTGCGACGACGCATCCAAGAAGCGGGCA |
| | | AAGACAAGAGGACGCAAGCGCCGCTCTCCTGAGAATATCGAGATCACCGTGACCC |
| | | TGTTTAAAGATCCTCACGCCGAGGAGTTCGAGGGCAAGGAGTGGACATTTGTGATC |
| | | GAGAATGAGTCCCCAAGGGGAAGGAAGAGACGGTCCGCCGCCTCTAGACCACGGC |
| | | TGGACCCTTGGAAGCTGGTGTCCTTTTCTCGCACCCTGACCATCAGCCCCCCTAGCC |
| | | GCCCAGATACCCCTGAGTCCCCTCGCGGGAGAAAGAGAAGATCCTCCAGCAAGAC |
| | | AACATCCGTGCTGCAGTGGGCCGAGAAGGGCTATTACACCCGCTCTAACAATCTGG |
| | | TGACACTGGAGAATGGCAAGCAGCTGACCGTGCGGGGCAGGAAGCGCAGAAGCCT |
| | | GGACTGGCAGCCTAGGGAGAATCCATTTCAGAATCTGAAGGTGCTGTCCGTGAGCG |
| | | ATCAGCAGCAGAATTTTCTGGAGCTGTGGAGCGAGATCCTGAGAGGCAGAAAGCG |
| | | GCGCAGCGAGAGATGGGAGTGCACCACCTACTGGTATATCTTCGTGTTCTGCTCCG |
| | | CCCACCCTGCAGTGACCGAGATGGCCCTGTTCGTGACAGTGTTCGGCCTGCGCGGG |
| | | AGGAAGAGGCGGTCCTTTTACAAGAAGTGCGCACAGCTGACAGTGAATCTGACAA |
| | | GATTCCCAGAGACCGTGCCTGGCGAGCTGGTGGTGCCCGTGGCAGGCTCCTGCGTG |
| | | GTGAGAGGCCGCAAGAGAAGGTCTAACAATGTGGACCTGATCCTGGCCACCCCAC |
| | | CATTTTCTAGACTGGAGAAACTGTACTCCGGCGAGGTGGTGAGAGGCAGGAAGCGC |
| | | AGGTCCAAGACCATGAAGGATTTTGTGCGGAGGAGGTGCTGGGCAAGGAAGTATA |
| | | AGCTGGTGACCTCTGGACCTTGGCTGGAGGTGCCCCCAATCGCACTGAGAAGGGGC |
| | | AGAAAGCGGAGGAGCGCCAGGAGAATGGCCTTTCTGGCCAAGAAGGGCTACAGGC |
| | | ACGATTCTTCCACAGCAGTGGTGGGGAGCCCCAGGGGCCACGGCCAGTCTAGAGA |
| | | GACCCGCGGCAGGAAGCGGAGATCTTGGCGGCGGGCACAGAAGCTGCATTTCCTG |
| | | CAGAGGGGGGGGCTGAAGCAGCTGAGCGCCCGGGTGAGGCCAGAGCTGAAAACCA |
| | | AGCAGCAGATCCTGAGGGGCCGCAAACGGAGGAGCTCTGGCCGGCTGGATCAGCT |
| | | GCTGTCTCTGTCCATGGTGTGGGCCAACCACGTGTTCCTGGGATGTAGCTACAATAA |
| | | GGACCTGCTGGATAAGGTGATGAGGGGCAGGAAGAGGCGCAGCGAGCTGGGCTCC |
| | | CTGATCCGCCAGCTGTCCCTGTGCTCTGTGAAGCTGTACGCCGATCCATCTGTCCCA |
| | | GATGTGGTGATCGACATCCTGCAGCAGATCAGAGGCAGGAAGAGAAGAAGCCCTG |
| | | AGCTGTCTATCAGCAGCCTGACATCTCCTATCCCAGAGAACAGCACAGAAACCGAG |
| | | GTGGCCCTGTTCAGGATCAGAGACAGAGATTCCGGCGAGCGCGGCCGGAAGCGCA |
| | | GAAGCACCTCCGAGCTGCTGGGCGAGGGCGCCTACGCCAAGGTGCAGGGCGCAAT |
| | | GTCCCTGCAGAACGGAAAGGAGTACGCAGTGAAGATCATCGAGAAGCAGCGCGGC |
| | | AGGAAGAGACGCTCTGATTGGGAGGGCAACAGGGCATATAGCCAGTACGACAAGT |
| | | TCCACATCGGAAACGAGAAGCAGAACTATAGACTGTACCTGAAGGGCCACACAGG |
| | | CACCCGGGGCAGAAAGAGGCGGAGCCTGCGGCCAAAGCTGATCGAGGAGCTGGCC |
| | | GCAACCCGGATCGTGGACGTGTCCACCGGAGATTCCCACTGTCTGGCCCTGTCCCA |
| | | CGATAACGAGGTGAGAGGACGGAAGAGAAGGTCTTATGCCTACGACAAAAGAGCC |
| | | AACCCACAGGTGGGAGCCGCCTTCCCCCACATCAAGCACAACTACGAGTGTCTGGT |
| | | GTATGTGCAGCTGCCATTCATGCGCGGCAGAAAGAGACGCAGCAGGCGCTATCCTT |
| | | CTATGGCCCGCATCCACAGCATGACCATCGAGGCCCCCATCGCAAAGGACGGACTG |
| | | CGGCGCCTGAGCGGCAATGAGTACGTGTTTAGGGGCAGAAAGAGACGGTCCATCT |
| | | CCACAGCAGTGAGGCCCACCACACCCACAACCTGCAGCAGGAGGTGGTGGCGCCC |
| | | ATCCAGCGTGGGAGGCAGCGCCTCTGGCTCCAGGCCTCCATGATAACTCGAGtctagag |
| | | ggcccgtttaaacccgctgatcagcctcgactgtgccttctagttgccagccatctgttgtttgcccctc |
| | | ccccgtgccttccttgaccctggaaggtgccactcccactgtcctttcctaataaaatgaggaaattgca |
| | | tcgcattgtctgagtaggtgtcattctattctggggggtggggtggggcaggacagcaagggggaggatt |
| | | gggaagacaatagcaggcatgctggggatgcggtgggctctatggcttctactgggcggttttatggaca |
| | | gcaagcgaaccggaattgccagctggggcgccctctggtaaggttgggaagccctgcaaagtaaactgga |
| | | tggctttcttgccgccaaggatctgatggcgcaggggatcaagctctgatcaagagacaggatgaggatc |
| | | gtttcgcatgattgaacaagatggattgcacgcaggttctccggccgcttgggtggagaggctattcggc |
| | | tatgactgggcacaacagacaatcggctgctctgatgccgccgtgttccggctgtcagcgcaggggcgcc |
| | | cggttctttttgtcaagaccgacctgtccggtgccctgaatgaactgcaagacgaggcagcgcggctatc |
| | | gtggctggccacgacgggcgttccttgcgcagctgtgctcgacgttgtcactgaagcgggaagggactgg |
| | | ctgctattgggcgaagtgccggggcaggatctcctgtcatctcaccttgctcctgccgagaaagtatcca |
| | | tcatggctgatgcaatgcggcggctgcatacgcttgatccggctacctgcccattcgaccaccaagcgaa |
| | | acatcgcatcgagcgagcacgtactcggatggaagccggtcttgtcgatcaggatgatctggacgaagag |
| | | catcaggggctcgcgccagccgaactgttcgccaggctcaaggcgagcatgcccgacggcgaggatctcg |
| | | tcgtgacccatggcgatgcctgcttgccgaatatcatggtggaaaatggccgcttttctggattcatcga |
| | | ctgtggccggctgggtgtggcggaccgctatcaggacatagcgttggctacccgtgatattgctgaagag |
| | | cttggcggcgaatgggctgaccgcttcctcgtgctttacggtatcgccgctcccgattcgcagcgcatcg |
| | | ccttctatcgccttcttgacgagttcttctgaattattaacgcttacaatttcctgatgcggtattttct |
| | | ccttacgcatctgtgcggtatttcacaccgcatcaggtggcacttttcggggaaatgtgcgcggaacccc |
| | | tatttgtttatttttctaaatacattcaaatatgtatccgctcatgagacaataaccctgataaatgctt |
| | | caataatagcacgtgctaaaacttcatttttaatttaaaaggatctaggtgaagatcctttttgataatc |
| | | tcatgaccaaaatcccttaacgtgagttttcgttccactgagcgtcagaccccgtagaaaagatcaaagg |
| | | atcttcttgagatcctttttttctgcgcgtaatctgctgcttgcaaacaaaaaaaccaccgctaccagcg |
| | | gtggtttgtttgccggatcaagagctaccaactctttttccgaaggtaactggcttcagcagagcgcaga |
| | | taccaaatactgttcttctagtgtagccgtagttaggccaccacttcaagaactctgtagcaccgcctac |
| | | atacctcgctctgctaatcctgttaccagtggctgctgccagtggcgataagtcgtgtcttaccgggttg |
| | | gactcaagacgatagttaccggataaggcgcagcggtcgggctgaacggggggttcgtgcacacagccca |
| | | gcttggagcgaacgacctacaccgaactgagatacctacagcgtgagctatgagaaagcgccacgcttcc |
| | | cgaagggagaaaggcggacaggtatccggtaagcggcagggtcggaacaggagagcgcacgagggagctt |
| | | ccagggggaaacgcctggtatctttatagtcctgtcgggtttcgccacctctgacttgagcgtcgatttt |
| | | tgtgatgctcgtcaggggggcggagcctatggaaaaacgccagcaacgcggcctttttacggttcctggc |
| | | cttttgctggccttttgctcacatgttctt |

In some embodiments, the nucleic acid molecule or the pharmaceutical composition comprises a DNA backbone that comprises all of the lowercase basepairs from any of the above-identified plasmids, wherein a first lowercase backbone sequence and a second lowercase backbone sequence flank the expressible nucleic acid sequence encoding the plurality of tumor-specific antigen sequences, such as Formula I, Formula I (a), Formula II or Formula III (a). In some embodiments, the DNA backbone segment or segments of the nucleic acid molecule comprises at least one segment that comprises at least about 70%, 75%, 80%, 85%, 90%, 91%, 92%, 93%, 94%, 95%, 96%, 97%, 98%, 99% sequence identity to the lowercases sequences presented above.

In some embodiments, a nucleic acid molecule comprises a nucleic acid sequence comprising Formula I ([(AEDn)-(linker)]n-[AEDn+1]), wherein the each linker is independently selectable from about 0 to about 25 natural or non-natural nucleic acids in length. In some embodiments, a nucleic acid molecule comprises a nucleic acid sequence comprising Formula I ([(AEDn)-(linker)]n-[AEDn+1]), wherein the each linker is independently selectable from about 0 to about 25 natural or non-natural nucleic acids in length, about 0 to about 25, about 1 to about 25, about 2 to about 25, about 3 to about 25, about 4 to about 25, about 5 to about 25, about 6 to about 25, about 7 to about 25, about 8 to about 25, about 9 to about 25, about 10 to about 25, about 11 to about 25, about 12 to about 25, about 13 to about 25, about 14 to about 25, about 15 to about 25, about 16 to about 25, about 17 to about 25, about 18 to about 25, about 19 to about 25, about 20 to about 25, about 21 to about 25, about 22 to about 25, about 23 to about 25, about 24 to about 25. In some embodiments, each linker is about 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25 natural or non-natural nucleic acids in length. In some embodiments, each linker is about 21 natural or non-natural nucleic acids in length. In certain embodiments, two linkers can be used together, in a fusion. Accordingly, in some embodiments, the first linker is independently selectable from about 0 to about 25 natural or non-natural nucleic acids in length, about 0 to about 25, about 1 to about 25, about 2 to about 25, about 3 to about 25, about 4 to about 25, about 5 to about 25, about 6 to about 25, about 7 to about 25, about 8 to about 25, about 9 to about 25, about 10 to about 25, about 11 to about 25, about 12 to about 25, about 13 to about 25, about 14 to about 25, about 15 to about 25, about 16 to about 25, about 17 to about 25, about 18 to about 25, about 19 to about 25, about 20 to about 25, about 21 to about 25, about 22 to about 25, about 23 to about 25, about 24 to about 25 natural or non-natural nucleic acids in length. In some embodiments, the second linker is independently selectable from about 0 to about 25, about 1 to about 25, about 2 to about 25, about 3 to about 25, about 4 to about 25, about 5 to about 25, about 6 to about 25, about 7 to about 25, about 8 to about 25, about 9 to about 25, about 10 to about 25, about 11 to about 25, about 12 to about 25, about 13 to about 25, about 14 to about 25, about 15 to about 25, about 16 to about 25, about 17 to about 25, about 18 to about 25, about 19 to about 25, about 20 to about 25, about 21 to about 25, about 22 to about 25, about 23 to about 25, about 24 to about 25 natural or non-natural nucleic acids in length. In some embodiments, the first linker is independently selectable from a linker that is about 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25 natural or non-natural nucleic acids in length. In some embodiments, the second linker is independently selectable from a linker that is about 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25 natural or non-natural nucleic acids in length.

In some embodiments, the at least one linker comprises from about 15 to about 300 nucleotides and encodes an amino acid cleavage site. In some embodiments, each linker positioned between each AED is the same nucleotide sequence comprising from about 15 to about 120 nucleotides and encodes an amino acid cleavage site

In some embodiments, the formula (e.g. [(AEDn)-(linker)]n-[AEDn+1]) comprises at least 1, 2, 3, 4, 5, 6, 7, 8, 9, 10 or more linkers.

In some embodiments, the formula comprises at least a first linker and a second linker.

In some embodiments, the formula comprises at least a first linker, a second linker, and a third linker. In some embodiments, the formula comprises at least a first linker, a second linker, a third linker, and a fourth linker. In some embodiments, the formula comprises at least a first linker, a second linker, a third linker, a fourth linker, and a fifth linker. In some embodiments, the formula comprises at least nineteen linkers flanking at least twenty antigen AED domains.

In a further embodiment, the at least one linker comprises a furin protease cleavage site.

Furin is a protease which resides in the trans-Golgi network of eukaryotic cells. Its function is to cleave proteins at a step just prior to their delivery to their final cellular destination. Furin recognizes a consensus amino acid sequence, RXRR, RXRK or KXKR (where X is any amino acid, Moehring et al., 1993, incorporated by reference in its entirety herein) and cuts proteins which contain these sequences when they reach the trans-Golgi network. Furin is a Ca2+-dependent serine endoprotease that cleaves protein precursors with a high specificity after the multiple basic motifs shown in Table 9 below.

TABLE 9

| | |
|---|---|
| Canonic | -R^{P4}-X^{P3}-(K/R)^{P2}-R^{P1}↓X^{P1′}-X^{P2′}-X^{P3′}-X^{P4′} |
| Alternative | -R^{P6}-X^{P5}-X^{P4}-X^{P3}-(K/R)^{P2}-R^{P1}↓X^{P1′}-X^{P2′}-X^{P3′}-X^{P4′} |
| Minimal | -R^{P4}-X^{P3}-X^{P2}-R^{P1}↓X^{P1′}-X^{P2′}-X^{P3′}-X^{P4′} |

In certain embodiments, the one or plurality of nucleic acid molecules encode a furin-sensitive cleavage site selected from the sequence R-X-[R/K]-R, where R denotes arginine, X is any amino acid, and K is lysine. The “R/K” indicates that this amino acid may be either arginine or lysine.

In certain embodiments, a furin cleavage site is introduced after the antigen domain 1 and/or the antigen domain 2 (e.g. [(AEDn)-(linker)]n-[AEDn+1]).

In some embodiments, the at least one linker comprises from about 15 to about 300 nucleotides and encodes a cleavage site, wherein the at least one linker comprises a 2A cleavage site. In some embodiments, the at least one linker comprises from about 15 to about 300 nucleotides and encodes a cleavage site, wherein the at least one linker comprises a porcine teschovirus-1 2A (P2A) cleavage site.

A 2A peptide is a “self-cleaving” small peptide. The average length of 2A peptides is 18-22 amino acids. The designation “2A” refers to a specific region of picornavirus polyproteins. Of the 2A peptides identified to date, four are widely used in research: FMDV 2A (abbreviated herein as F2A); equine rhinitis A virus (ERAV) 2A (E2A); porcine teschovirus-1 2A (P2A) and Thosea asigna virus 2A (T2A). The former three viruses belong to picornaviruses and the latter is an insect virus. DNA and corresponding amino acid sequences of various 2A peptides are shown below in Table 10. Underlined sequences encode amino acids GSG, which were added to improve cleavage efficiency. P2A indicates porcine teschovirus-1 2A; T2A, Thosea asigna virus 2A; E2A, equine rhinitis A virus (ERAV) 2A; F2A, FMDV 2A.

Table 10 discloses SEQ ID NOS 368-375,
respectively, in order of appearance.

| | |
|---|---|
| P2A | GGA AGC GGA GCT ACT AAC TTC AGC CTG CTG AAG CAG GCT GGA |
| | G S G A T N F S L L K Q A G |
| | GAC GTG GAG GAG AAC CCT GGA CCT |
| | D V E E N P G P |
| T2A | GGA AGC GGA GAG GGC AGA GGA AGT CTG CTA ACA TGC GGT GAC |
| | G S G E G R G S L L T C G D |
| | GTC GAG GAG AAT CCT GGA CCT |
| | V E E N P G P |
| E2A | GGA AGC GGA CAG TGT ACT AAT TAT GCT CTC TTG AAA TTG GCT |
| | G S G Q C T N Y A L L K L A |
| | GGA GAT GTT GAG AGC AAC CCT GGA CCT |
| | G D V E S N P G P |
| F2A | GGA AGC GGA GTG AAA CAG ACT TTG AAT TTT GAC CTT CTC AAG |
| | G S G V K Q T L N F D L L K |
| | TTG GCG GGA GAC GTG GAG TCC AAC CCT GGA CCT |
| | L A G D V E S N P G P |

In some embodiments, the formula comprises at least a first linker and a second linker, wherein the first and second linker comprise a furin protease cleavage site.

In some embodiments, the formula comprises at least a first linker, a second linker, and a third linker, wherein the first, second and third linker comprise a furin protease cleavage site.

In some embodiments, the formula comprises at least a first linker, a second linker, a third linker, and a fourth linker, wherein the first, second, third and fourth linker comprise a furin protease cleavage site.

In some embodiments, the formula comprises at least a first linker, a second linker, a third linker, a fourth linker, and a fifth linker, wherein the first, second, third, fourth and fifth linker comprise a furin protease cleavage site.

In some embodiments, the formula comprises at least a first linker, a second linker, a third linker, a fourth linker, and a fifth linker, wherein the first, second, third, fourth and fifth linker comprise a furin protease cleavage site.

In some embodiments, the formula comprises at least a first linker and a second linker, wherein the first and second linker comprise a P2A protease cleavage site.

In some embodiments, the formula comprises at least a first linker, a second linker, and a third linker, wherein the first, second and third linker comprise a P2A cleavage site.

In some embodiments, the formula comprises at least a first linker, a second linker, a third linker, and a fourth linker, wherein the first, second, third and fourth linker comprise a P2A cleavage site.

In some embodiments, the formula comprises at least a first linker, a second linker, a third linker, a fourth linker, and a fifth linker, wherein the first, second, third, fourth and fifth linker comprise a P2A cleavage site.

In some embodiments, the formula comprises at least a first linker, a second linker, a third linker, a fourth linker, a fifth linker, or more wherein the first, second, third, fourth, fifth linker, or more linkers comprise a P2A protease cleavage site.

In some embodiments, the formula comprises at least a first linker and a second linker, wherein at least one of the first or second linkers comprise a furin protease cleavage site.

In some embodiments, the formula comprises at least a first linker, a second linker, and a third linker, wherein at least one of the first, second or third linkers comprise a furin protease cleavage site.

In some embodiments, the formula comprises at least a first linker, a second linker, a third linker, and a fourth linker, at least one of the first, second, third or fourth linkers comprise a furin protease cleavage site.

In some embodiments, the formula comprises at least a first linker, a second linker, a third linker, a fourth linker, and a fifth linker, at least one of the first, second, third, fourth or fifth linkers comprise a furin protease cleavage site.

In some embodiments, the formula comprises at least a first linker and a second linker, wherein at least one of the first or second linkers comprise a P2A protease cleavage site.

In some embodiments, the formula comprises at least a first linker, a second linker, and a third linker, wherein at least one of the first, second or third linkers comprise a P2A protease cleavage site.

In some embodiments, the formula comprises at least a first linker, a second linker, a third linker, and a fourth linker, at least one of the first, second, third or fourth linkers comprise a P2A protease cleavage site.

In some embodiments, the formula comprises at least a first linker, a second linker, a third linker, a fourth linker, and a fifth linker, at least one of the first, second, third, fourth or fifth linkers comprise a P2A protease cleavage site.

Nucleic acid molecules useful in the methods of the disclosure include any nucleic acid molecule that encodes a neoantigen, or a fragment thereof; any nucleic acid that encodes a linker, any nucleic acid that encodes a regulatory sequence, any nucleic acid that encodes a leader sequence. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. In some embodiments, the some embodiments, the nucleic acid sequence or molecules of the disclosure relate to nucleic acid sequences comprising a nucleic acid sequence at least about 70%, 80%, 85, 90%, 95%, 96%, 97%, 98%, 99% or 100% sequence identity to the sequences identified in the Figures.

In some embodiments, an exemplary leader sequence is an IgE leader amino acid sequence as set forth in the sequence below and described in US20160175427, incorporated by reference in its entirety herein.

In some embodiments, the nucleic acid comprises a coding region consisting of any of Formulae I, I (a) I (b), II (a) and/or III (a) and one or a plurality of leader sequences. In some embodiments, the leader sequence is an IgE leader sequence: Met Asp Trp Thr Trp Ile Leu Phe Leu Val Ala Ala Ala Thr Arg Val (SEQ ID NO:55) or a leader sequence that is a functional fragment thereof comprising at least about 70%, 80%, 85%, 90%, 95%, 96%, 97%, 98% or 99% homologous to the IgE leader sequence identified in the aforementioned sentence. In some embodiments, the nucleic acid sequence or molecules of the disclosure relate to nucleic acid sequences comprising a leader with at least about 70%, 80%, 85%, 90%, 95%, 96%, 97%, 98%, 99% or 100% sequence identity to SEQ ID NO:55.

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30 °C, more preferably of at least about 37 °C, and most preferably of at least about 42 °C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In some embodiments, hybridization will occur at 30 °C in 750 mM NaCl, 75 mM trisodium citrate, and 1% SDS. In some embodiments, hybridization will occur at 37 °C in 500 mM NaCl, 50 mM trisodium citrate, 1% SDS, 35% formamide, and 100 μg/ml denatured salmon sperm DNA (ssDNA). In some embodiments, hybridization will occur at 42 °C in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25 °C, more preferably of at least about 42 °C, and even more preferably of at least about 68 °C. In some embodiments, wash steps will occur at 25 °C in 30 mM NaCl, 3 mM trisodium citrate, and 0.1% SDS. In some embodiments, wash steps will occur at 42 °C in 15 mM NaCl, 1.5 mM trisodium citrate, and 0.1% SDS. In some embodiments, wash steps will occur at 68 °C in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

The nucleic acid sequences may be used in association with other polynucleotide sequences coding for regulatory proteins that control the expression of the neo antigen sequence. For example, the nucleic acid molecule according to the disclosure may additionally contain recognition, regulatory, leader and promoter sequences.

In some embodiments, the nucleic acid molecule further comprises at least one regulatory sequence, wherein at least one nucleic acid sequence of Formula I is operably linked to the regulatory sequence.

In some embodiments, the nucleic acid molecule further comprises a leader sequence.

In some embodiments, an exemplary leader sequence is an IgE leader amino acid sequence as described in US20160175427, incorporated by reference in its entirety herein.

In some embodiments, the nucleic acid molecule comprises a nucleic acid sequence comprising Formula I ([(AEDn)-(linker)]n-[AEDn+1]), wherein the antigen expression domain 1 is independently selectable from about 12 to about 15,000 nucleotides in length and encodes an epitope from one or a plurality of cancer cells from a subject; and the antigen expression domain 2 is independently selectable from about 12 to about 15,000 nucleotides in length and encodes an epitope from one or a plurality of cancer cells from the subject.

In some embodiments, the nucleic acid molecule comprising a nucleic acid sequence comprising Formula I ([(AEDn)-(linker)]n-[AEDn+1]) is in an amount sufficient to elicit a cellular immune response. A “cellular immune response” is meant to include a cellular response directed to cells characterized by presentation of an antigen with class I or class II MHC. The cellular response relates to cells called T cells or T-lymphocytes which act as either “helpers” or “killers”. The helper T cells (also termed CD4+ T cells) play a central role by regulating the immune response and the killer cells (also termed cytotoxic T cells, cytolytic T cells, CD8+ T cells or CTLs) kill diseased cells such as cancer cells, preventing the production of more diseased cells. In some embodiments, the present disclosure involves the stimulation of an anti-tumor CTL response against tumor cells expressing one or more tumor expressed antigens and preferably presenting such tumor expressed antigens with class I MHC.

In some embodiments, the nucleic acid molecule comprising a nucleic acid sequence comprising Formula I ([(AEDn)-(linker)]n-[AEDn+1]) is in an amount sufficient to elicit a CD8+ T cell response against any one or plurality of amino acid sequences encoded by one or plurality of antigen expression domains. In some embodiments, the nucleic acid molecule comprising a nucleic acid sequence comprising Formula I ([(AEDn)-[AEDn+1]) is in an amount sufficient to elicit a CD8+T and/or CD4+ T cell response against any one or plurality of amino acid sequences encoded by one or plurality of antigen expression domains.

In some embodiments, the nucleic acid molecule comprising a nucleic acid sequence comprising Formula I ([(AEDn)-(linker)]n-[AEDn+1]) is in an amount sufficient to elicit a CD4+ T cell response against any one or plurality of amino acid sequences encoded by one or plurality of antigen expression domains. In some embodiments, the nucleic acid molecule comprising a nucleic acid sequence comprising Formula I ([(AEDn)-(linker)]n-[AEDn+1]) is in an amount sufficient to elicit a subpopulation of T cells that are greater than at least about 40% CD4+ T cells in response against any one or plurality of amino acid sequences encoded by one or plurality of antigen expression domains as compared to the response generated without the nucleic acid sequences disclosed herein. In some embodiments, the nucleic acid molecule comprising a nucleic acid sequence comprising Formula I ([(AEDn)-(linker)]n-[AEDn+1]) is in an amount sufficient to elicit a subpopulation of T cells that are greater than at least about 40% CD8+ T cells in response against any one or plurality of amino acid sequences encoded by one or plurality of antigen expression domains as compared to the response generated without the nucleic acid sequences disclosed herein. In some embodiments, the nucleic acid molecule comprising a nucleic acid sequence comprising Formula I ([(AEDn)-(linker)]n-[AEDn+1]) is in an amount sufficient to elicit a subpopulation of T cells that comprise greater than at least about 40% CD4+ T cells and that comprise greater than 40% CD8+ T cells in response against any one or plurality of amino acid sequences encoded by one or plurality of antigen expression domains as compared to the response generated without the nucleic acid sequences disclosed herein.

In a still further aspects, the nucleic acid molecule described in any of the aspects and embodiments herein is a plasmid. In certain embodiments, an expression vector comprises the nucleic acid molecule described in any of the aspects and embodiments. In certain embodiments, the nucleic acid expression vector is a plasmid. In some embodiments, the vector is capable of expressing one or a plurality of consensus neoantigen sequences in the cell of a mammal in a quantity effective to elicit an antigen-specific immune response in the mammal against one or a plurality of the consense neoantigens. In some embodiments, the vector is recombinant. In some embodiments, the vector comprises a heterologous nucleic acid encoding one or a plurality of neoantigens. In some embodiments, the vector is a plasmid. In some embodiments, the vector can be useful for transfecting cells with nucleic acid encoding a neoantigen, which the transformed host cell is cultured and maintained under conditions wherein expression of the neoantigen takes place. In some embodiments, the vector is capable of expressing one or a plurality of neoantigen sequences in the cell of a mammal in a quantity effective to elicit an immune response in the mammal. In some embodiments, a cell comprising the nucleic acid molecule is capable of expressing one or a plurality of consensus neoantigen sequences in the cell of a mammal in a quantity effective to elicit an immune response in the mammal that shrinks a tumor by more than about 5, 10, 15, 20, 30, 40, 50, 60, 70 or more percent. In some embodiments, a cell comprising the nucleic acid molecule is capable of expressing one or a plurality of neoantigen amino acid sequences in the cell of a mammal in a quantity effective to elicit an clonal expansion of CD8+ T cells from about 0.1 to about 50% of the total T cell stimulation against the one or plurality of neoantigens.

In some embodiments, the vector comprises heterologous nucleic acid encoding a neoantigen and can further comprise an initiation codon, which can be upstream of the neoantigen coding sequence, and a stop codon, which can be downstream of the neoantigen coding sequence. The initiation and termination codon can be in frame with the neoantigen coding sequence. The vector can also comprise a promoter that is operably linked to the neoantigen coding sequence. The promoter operably linked to the neoantigen coding sequence can be a promoter from simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter can also be a promoter from a human gene such as human actin, human myosin, human hemoglobin, human muscle creatine, or human metalothionein. The promoter can also be a tissue specific promoter, such as a muscle or skin specific promoter, natural or synthetic. Examples of such promoters are described in US patent application publication no. US20040175727, the contents of which are incorporated herein in its entirety.

The vector can also comprise a polyadenylation signal, which can be downstream of the HA coding sequence. The polyadenylation signal can be a SV40 polyadenylation signal, LTR polyadenylation signal, bovine growth hormone (bGH) polyadenylation signal, human growth hormone (hGH) polyadenylation signal, or human β-globin polyadenylation signal. The SV40 polyadenylation signal can be a polyadenylation signal from a pCEP4 vector (Invitrogen, San Diego, Calif.).

The vector can also comprise an enhancer upstream of the neoantigen coding. The enhancer can be necessary for DNA expression. The enhancer can be human actin, human myosin, human hemoglobin, human muscle creatine or a viral enhancer such as one from CMV, HA, RSV or EBV. Polynucleotide function enhances are described in U.S. Pat. Nos. 5,593,972, 5,962,428, and WO94/016737, the contents of each are fully incorporated by reference in their entireties.

The vector can also comprise a mammalian origin of replication in order to maintain the vector extrachromosomally and produce multiple copies of the vector in a cell. In some embodiments, the vector is LLC, TC1, ID8, pGX0001, pGX4501, pGX4503, pGX4504, pGX4505, pGX4506 and/or pGX6001 or comprises any one or more regulatory or non-coding sequences of LLC, TC1, ID8, pGX0001, pGX4501, pGX4503, pGX4504, pGX4505, pGX4506 and/or pGX6001. In some embodiments, the vector comprises the sequence that is pVAX1. In some embodiments, the backbone of the vector is pAV0242. In some embodiments, the vector can be a replication-defective adenovirus type 5 (Ad5) vector.

The vector can also comprise a regulatory sequence, which can be well suited for gene expression in a mammalian or human cell into which the vector is administered. The neoantigen coding sequence can comprise a codon, which can allow more efficient transcription of the coding sequence in the host cell.

The vector can be pSE420 (Invitrogen, San Diego, Calif.), which can be used for protein production in Escherichia coli (E. coli). The vector can also be pYES2 (Invitrogen, San Diego, Calif.), which can be used for protein production in Saccharomyces cerevisiae strains of yeast. The vector can also be of the MAXBAC™ complete baculovirus expression system (Invitrogen, San Diego, Calif.), which can be used for protein production in insect cells. The vector can also be pcDNA I or pcDNA3 (Invitrogen, San Diego, Calif.), which can be used for protein production in mammalian cells such as Chinese hamster ovary (CHO) cells. The vector can be expression vectors or systems to produce protein by routine techniques and readily available starting materials including Sambrook et al., Molecular Cloning: A Laboratory Manual, Second Ed., Cold Spring Harbor (1989), which is incorporated fully by reference.

Expression vectors for different cell types are well known in the art and can be selected without undue experimentation. Generally, the DNA is inserted into an expression vector, such as a plasmid, in proper orientation and correct reading frame for expression. If necessary, the DNA may be linked to the appropriate transcriptional and translational regulatory control nucleotide sequences recognized by the desired host (e.g., bacteria), although such controls are generally available in the expression vector. The vector is then introduced into the host bacteria for cloning using standard techniques (see, e.g., Sambrook et al. (1989) Molecular Cloning, A Laboratory Manual, Cold Spring Harbor Laboratory, Cold Spring Harbor, N.Y.).

In some embodiments, the nucleic acid sequence of Formula I is positioned with the multiple cloning site of a plasmid selected from the group consisting of LLC, TC1, ID8, pGX0001, pGX4501, pGX4503, pGX4504, pGX4505, pGX4506 and/or pGX6001.

In some embodiments, the nucleic acid sequence of Formula I is positioned with the multiple cloning site of LLC. In some embodiments, the nucleic acid sequence of Formula I is positioned with the multiple cloning site of TC1. In some embodiments, the nucleic acid sequence of Formula I is positioned with the multiple cloning site of ID8. In some embodiments, the nucleic acid sequence of Formula I is positioned with the multiple cloning site of pGX0001. In some embodiments, the nucleic acid sequence of Formula I is positioned with the multiple cloning site of pGX4501. In some embodiments, the nucleic acid sequence of Formula I is positioned with the multiple cloning site of pGX4503. In some embodiments, the nucleic acid sequence of Formula I is positioned with the multiple cloning site of pGX4504. In some embodiments, the nucleic acid sequence of Formula I is positioned within the multiple cloning site of pGX4505. In some embodiments, the nucleic acid sequence of Formula I is positioned with the multiple cloning site of pGX4506. In some embodiments, the plasmid is pGX4505 or a sequence that is 70%, 80%, 90%, 91%, 92%, 93%, 94%, 95%, 96%, 97%, 98%, 99% homologous to each of the above-identified nucleotide sequences. In some embodiments, the plasmid is pGX0001 or a sequence that comprises about 70%, 80%, 90%, 91%, 92%, 93%, 94%, 95%, 96%, 97%, 98% or 99% sequence identity to each of the above-identified nucleotide sequences. In some embodiments, the plasmid is pGX6001 or a sequence that comprises about 70%, 80%, 90%, 91%, 92%, 93%, 94%, 95%, 96%, 97%, 98% or 99% sequence identity to each of the above-identified nucleotide sequences.

TABLE 11
Plasmid Backbone Sequences

| | |
|---|---|
| SEQ ID | gctgcttcgcgatgtacgggccagatatacgcgttgacattgattattgactagtt |
| NO: 63 | attaatagtaatcaattacggggtcattagttcatagcccatatatggagttccgc |
| pGX0001 | gttacataacttacggtaaatggcccgcctggctgaccgcccaacgacccccgccc |
| full | attgacgtcaataatgacgtatgttcccatagtaacgccaatagggactttccatt |
| plasmid | gacgtcaatgggtggagtatttacggtaaactgcccacttggcagtacatcaagtg |
| DNA | tatcatatgccaagtacgccccctattgacgtcaatgacggtaaatggcccgcctg |
| sequence: | gcattatgcccagtacatgaccttatgggactttcctacttggcagtacatctacg |
| | tattagtcatcgctattaccatggtgatgcggttttggcagtacatcaatgggcgt |
| | ggatagcggtttgactcacggggatttccaagtctccaccccattgacgtcaatgg |
| | gagtttgttttggcaccaaaatcaacgggactttccaaaatgtcgtaacaactccg |
| | ccccattgacgcaaatgggcggtaggcgtgtacggtgggaggtctatataagcaga |
| | gctctctggctaactagagaacccactgcttactggcttatcgaaattaatacgac |
| | tcactatagggagacccaagctggctagcgtttaaacttaagcttggtaccgagct |
| | cggatccactagtccagtgtggtggaattctgcagatatccagcacagtggcggcc |
| | gctcgagtctagagggcccgtttaaacccgctgatcagcctcgactgtgccttcta |
| | gttgccagccatctgttgtttgcccctcccccgtgccttccttgaccctggaaggt |
| | gccactcccactgtcctttcctaataaaatgaggaaattgcatcgcattgtctgag |
| | taggtgtcattctattctggggggtggggtggggcaggacagcaagggggaggatt |
| | gggaagacaatagcaggcatgctggggatgcggtgggctctatggcttctactggg |
| | cggttttatggacagcaagcgaaccggaattgccagctggggcgccctctggtaag |
| | gttgggaagccctgcaaagtaaactggatggctttcttgccgccaaggatctgatg |
| | gcgcaggggatcaagctctgatcaagagacaggatgaggatcgtttcgcatgattg |
| | aacaagatggattgcacgcaggttctccggccgcttgggtggagaggctattcggc |
| | tatgactgggcacaacagacaatcggctgctctgatgccgccgtgttccggctgtc |
| | agcgcaggggcgcccggttctttttgtcaagaccgacctgtccggtgccctgaatg |
| | aactgcaagacgaggcagcgcggctatcgtggctggccacgacgggcgttccttgc |
| | gcagctgtgctcgacgttgtcactgaagcgggaagggactggctgctattgggcga |
| | agtgccggggcaggatctcctgtcatctcaccttgctcctgccgagaaagtatcca |
| | tcatggctgatgcaatgcggcggctgcatacgcttgatccggctacctgcccattc |
| | gaccaccaagcgaaacatcgcatcgagcgagcacgtactcggatggaagccggtct |
| | tgtcgatcaggatgatctggacgaagagcatcaggggctcgcgccagccgaactgt |
| | tcgccaggctcaaggcgagcatgcccgacggcgaggatctcgtcgtgacccatggc |
| | gatgcctgcttgccgaatatcatggtggaaaatggccgcttttctggattcatcga |
| | ctgtggccggctgggtgtggcggaccgctatcaggacatagcgttggctacccgtg |
| | atattgctgaagagcttggcggcgaatgggctgaccgcttcctcgtgctttacggt |
| | atcgccgctcccgattcgcagcgcatcgccttctatcgccttcttgacgagttctt |
| | ctgaattattaacgcttacaatttcctgatgcggtattttctccttacgcatctgt |
| | gcggtatttcacaccgcatcaggtggcacttttcggggaaatgtgcgcggaacccc |
| | tatttgtttatttttctaaatacattcaaatatgtatccgctcatgagacaataac |
| | cctgataaatgcttcaataatagcacgtgctaaaacttcatttttaatttaaaagg |
| | atctaggtgaagatcctttttgataatctcatgaccaaaatcccttaacgtgagtt |
| | ttcgttccactgagcgtcagaccccgtagaaaagatcaaaggatcttcttgagatc |
| | ctttttttctgcgcgtaatctgctgcttgcaaacaaaaaaaccaccgctaccagcg |
| | gtggtttgtttgccggatcaagagctaccaactctttttccgaaggtaactggctt |
| | cagcagagcgcagataccaaatactgttcttctagtgtagccgtagttaggccacc |
| | acttcaagaactctgtagcaccgcctacatacctcgctctgctaatcctgttacca |
| | gtggctgctgccagtggcgataagtcgtgtcttaccgggttggactcaagacgata |
| | gttaccggataaggcgcagcggtcgggctgaacggggggttcgtgcacacagccca |
| | gcttggagcgaacgacctacaccgaactgagatacctacagcgtgagctatgagaa |
| | agcgccacgcttcccgaagggagaaaggcggacaggtatccggtaagcggcagggt |
| | cggaacaggagagcgcacgagggagcttccagggggaaacgcctggtatctttata |
| | gtcctgtcgggtttcgccacctctgacttgagcgtcgatttttgtgatgctcgtca |
| | ggggggcggagcctatggaaaaacgccagcaacgcggcctttttacggttcctggc |
| | cttttgctggccttttgctcacatgttctt |
| SEQ ID | gctgcttcgcgatgtacgggccagatatacgcgttgacattgattattgactagtt |
| NO: 64 | attaatagtaatcaattacggggtcattagttcatagcccatatatggagttccgc |
| pGX0001 | gttacataacttacggtaaatggcccgcctggctgaccgcccaacgacccccgccc |
| first DNA | attgacgtcaataatgacgtatgttcccatagtaacgccaatagggactttccatt |
| backbone | gacgtcaatgggtggagtatttacggtaaactgcccacttggcagtacatcaagtg |
| | tatcatatgccaagtacgccccctattgacgtcaatgacggtaaatggcccgcctg |
| | gcattatgcccagtacatgaccttatgggactttcctacttggcagtacatctacg |
| | tattagtcatcgctattaccatggtgatgcggttttggcagtacatcaatgggcgt |
| | ggatagcggtttgactcacggggatttccaagtctccaccccattgacgtcaatgg |
| | gagtttgttttggcaccaaaatcaacgggactttccaaaatgtcgtaacaactccg |
| | ccccattgacgcaaatgggcggtaggcgtgtacggtgggaggtctatataagcaga |
| | gctctctggctaactagagaacccactgcttactggcttatcgaaattaatacgac |
| | tcactatagggagacccaagctg |
| SEQ ID | ccgctgatcagcctcgactgtgccttctagttgccagccatctgttgtttgcccct |
| NO: 65 | cccccgtgccttccttgaccctggaaggtgccactcccactgtcctttcctaataa |
| pGX0001 | aatgaggaaattgcatcgcattgtctgagtaggtgtcattctattctggggggtgg |
| Second | ggtggggcaggacagcaagggggaggattgggaagacaatagcaggcatgctgggg |
| DNA | atgcggtgggctctatggcttctactgggcggttttatggacagcaagcgaaccgg |
| backbone | aattgccagctggggcgccctctggtaaggttgggaagccctgcaaagtaaactgg |
| | atggctttcttgccgccaaggatctgatggcgcaggggatcaagctctgatcaaga |
| | gacaggatgaggatcgtttcgcatgattgaacaagatggattgcacgcaggttctc |
| | cggccgcttgggtggagaggctattcggctatgactgggcacaacagacaatcggc |
| | tgctctgatgccgccgtgttccggctgtcagcgcaggggcgcccggttctttttgt |
| | caagaccgacctgtccggtgccctgaatgaactgcaagacgaggcagcgcggctat |
| | cgtggctggccacgacgggcgttccttgcgcagctgtgctcgacgttgtcactgaa |
| | gcgggaagggactggctgctattgggcgaagtgccggggcaggatctcctgtcatc |
| | tcaccttgctcctgccgagaaagtatccatcatggctgatgcaatgcggcggctgc |
| | atacgcttgatccggctacctgcccattcgaccaccaagcgaaacatcgcatcgag |
| | cgagcacgtactcggatggaagccggtcttgtcgatcaggatgatctggacgaaga |
| | gcatcaggggctcgcgccagccgaactgttcgccaggctcaaggcgagcatgcccg |
| | acggcgaggatctcgtcgtgacccatggcgatgcctgcttgccgaatatcatggtg |
| | gaaaatggccgcttttctggattcatcgactgtggccggctgggtgtggcggaccg |
| | ctatcaggacatagcgttggctacccgtgatattgctgaagagcttggcggcgaat |
| | gggctgaccgcttcctcgtgctttacggtatcgccgctcccgattcgcagcgcatc |
| | gccttctatcgccttcttgacgagttcttctgaattattaacgcttacaatttcct |
| | gatgcggtattttctccttacgcatctgtgcggtatttcacaccgcatcaggtggc |
| | acttttcggggaaatgtgcgcggaacccctatttgtttatttttctaaatacattc |
| | aaatatgtatccgctcatgagacaataaccctgataaatgcttcaataatagcacg |
| | tgctaaaacttcatttttaatttaaaaggatctaggtgaagatcctttttgataat |
| | ctcatgaccaaaatcccttaacgtgagttttcgttccactgagcgtcagaccccgt |
| | agaaaagatcaaaggatcttcttgagatcctttttttctgcgcgtaatctgctgct |
| | tgcaaacaaaaaaaccaccgctaccagcggtggtttgtttgccggatcaagagcta |
| | ccaactctttttccgaaggtaactggcttcagcagagcgcagataccaaatactgt |
| | tcttctagtgtagccgtagttaggccaccacttcaagaactctgtagcaccgccta |
| | catacctcgctctgctaatcctgttaccagtggctgctgccagtggcgataagtcg |
| | tgtcttaccgggttggactcaagacgatagttaccggataaggcgcagcggtcggg |
| | ctgaacggggggttcgtgcacacagcccagcttggagcgaacgacctacaccgaac |
| | tgagatacctacagcgtgagctatgagaaagcgccacgcttcccgaagggagaaag |
| | gcggacaggtatccggtaagcggcagggtcggaacaggagagcgcacgagggagct |
| | tccagggggaaacgcctggtatctttatagtcctgtcgggtttcgccacctctgac |
| | ttgagcgtcgatttttgtgatgctcgtcaggggggcggagcctatggaaaaacgcc |
| | agcaacgcggcctttttacggttcctggccttttgctggccttttgctcacatgtt |
| | ctt |
| SEQ ID | gctagcgtttaaacttaagcttggtaccgagctcggatccactagtccagtgtggt |
| NO: 66 | ggaattctgcagatatccagcacagtggcggccgctcgagtctagagggcccgttt |
| Multiple | aaac |
| cloning | |
| site | |
| SEQ ID | aaatgggggcgctgaggtctgcctcgtgaagaaggtgttgctgactcataccaggc |
| NO: 67 | ctgaatcgccccatcatccagccagaaagtgagggagccacggttgatgagagctt |
| pGX6001 | tgttgtaggtggaccagttggtgattttgaacttttgctttgccacggaacggtct |
| full | gcgttgtcgggaagatgcgtgatctgatccttcaactcagcaaaagttcgatttat |
| plasmid | tcaacaaagccgccgtcccgtcaagtcagcgtaatgctctgccagtgttacaacca |
| sequence | attaaccaattctgcgttcaaaatggtatgcgttttgacacatccactatatatcc |
| | gtgtcgttctgtccactcctgaatcccattccagaaattctctagcgattccagaa |
| | gtttctcagagtcggaaagttgaccagacattacgaactggcacagatggtcataa |
| | cctgaaggaagatctgattgcttaactgcttcagttaagaccgacgcgctcgtcgt |
| | ataacagatgcgatgatgcagaccaatcaacatggcacctgccattgctacctgta |
| | cagtcaaggatggtagaaatgttgtcggtccttgcacacgaatattacgccatttg |
| | cctgcatattcaaacagctcttctacgataagggcacaaatcgcatcgtggaacgt |
| | ttgggcttctaccgatttagcagtttgatacactttctctaagtatccacctgaat |
| | cataaatcggcaaaatagagaaaaattgaccatgtgtaagcggccaatctgattcc |
| | acctgagatgcataatctagtagaatctcttcgctatcaaaattcacttccacctt |
| | ccactcaccggttgtccattcatggctgaactctgcttcctctgttgacatgacac |
| | acatcatctcaatatccgaatacggaccatcagtctgacgaccaagagagccataa |
| | acaccaatagccttaacatcatccccatatttatccaatattcgttccttaatttc |
| | atgaacaatcttcattctttcttctctagtcattattattggtccgttcataacac |
| | cccttgtattactgtttatgtaagcagacagttttattgttcatgatgatatattt |
| | ttatcttgtgcaatgtaacatcagagattttgagacacaacgtggctttccccggc |
| | ccatgaccaaaatcccttaacgtgagttttcgttccactgagcgtcagaccccgta |
| | gaaaagatcaaaggatcttcttgagatcctttttttctgcgcgtaatctgctgctt |
| | gcaaacaaaaaaaccaccgctaccagcggtggtttgtttgccggatcaagagctac |
| | caactctttttccgaaggtaactggcttcagcagagcgcagataccaaatactgtt |
| | cttctagtgtagccgtagttaggccaccacttcaagaactctgtagcaccgcctac |
| | atacctcgctctgctaatcctgttaccagtggctgctgccagtggcgataagtcgt |
| | gtcttaccgggttggactcaagacgatagttaccggataaggcgcagcggtcgggc |
| | tgaacggggggttcgtgcacacagcccagcttggagcgaacgacctacaccgaact |
| | gagatacctacagcgtgagctatgagaaagcgccacgcttcccgaagggagaaagg |
| | cggacaggtatccggtaagcggcagggtcggaacaggagagcgcacgagggagctt |
| | ccagggggaaacgcctggtatctttatagtcctgtcgggtttcgccacctctgact |
| | tgagcgtcgatttttgtgatgctcgtcaggggggcggagcctatggaaaaacgcca |
| | gcaacgcggcctttttacggttcctggccttttgctggccttttgctcacatgttc |
| | tttcctgcgttatcccctgattctgtggataaccgtattaccgcctttgagtgagc |
| | tgataccgctcgccgcagccgaacgaccgagcgcagcgagtcagtgagcgaggaag |
| | cggaagagcgcctgatgcggtattttctccttacgcatctgtgcggtatttcacac |
| | cgcatatggtgcactctcagtacaatctgctctgatgccgcatagttaagccagta |
| | tctgctccctgcttgtgtgttggaggtcgctgagtagtgcgcgagcaaaatttaag |
| | ctacaacaaggcaaggcttgaccgacaattgcatgaagaatctgcttagggttagg |
| | cgttttgcgctgcttcgcgatgtacgggccagatatagccgcggcatcgatgataa |
| | ttcggcttatttaaattccccagcatgcctgctattgtcttcccaatcctccccct |
| | tgctgtcctgccccaccccaccccccagaatagaatgacacctactcagacaatgc |
| | gatgcaatttcctcattttattaggaaaggacagtgggagtggcaccttccagggt |
| | caaggaaggcacgggggaggggcaaacaacagatggctggcaactagaaggcacag |
| | tcgaggctgatcagcgagctcggcgcgcctctcgagttactaactgcagggcacag |
| | atgcccattcgctccaagatgagctatagtagcggtcctgggcccgcacgctaatg |
| | ctggcatttttgcggcagatgaccgtggctgaggtcttgtccgtgaagactctatc |
| | tttcttttctctcttgctcttgccctggacctgaacgcagaatgtcagggagaagt |
| | aggaatgtggagtactccaggtgtcagggtactcccagctgacctccacctgccga |
| | gaattctttaatggcttcagctgcaagttcttgggtgggtcaggtttgatgatgtc |
| | cctgatgaagaagctgctggtgtagttttcatacttgagcttgtgaacggcatcca |
| | ccatgacctcaatgggcagactctcctcagcagctgggcaggcactgtcctcctgg |
| | cactccactgagtactcatactccttgttgtcccctctgactctctctgcagagag |
| | tgtagcagctccgcacgtcaccccttgggggtcagaagagcctctgctgcttttga |
| | cactgaatgtcaaatcagtactgattgtcgtcagccaccagcaggtgaaacgtcca |
| | gaataattcttggcctcgcatcttagaaaggtcttatttttgggttctttctggtc |
| | ctttaaaatatcagtggaccaaattccatcttcctttttgtgaagcagcaggagcg |
| | aatggcttagaacctcgcctcctttgtgacaggtgtactggccagcatctccaaac |
| | tctttgacttggatggtcagggttttgccagagcctaagacctcactgctctggtc |
| | caaggtccaggtgataccatcttcttcaggggtgtcacaggtgaggaccaccattt |
| | ctccaggggcatccggataccaatccaattctacgacataaacatctttcttcagt |
| | tcccatatggccacgaggggagatgccagaaaaaccagggaaaaccaagagatgac |
| | caactgctggtgacacatggtggctcgagtgggcccaagtttaaacgctcctccga |
| | cgtccccaggcagaatggcggttccctaaacgagcattgcttatatagacctccca |
| | ttaggcacgcctaccgcccatttacgtcaatggaacgcccatttgcgtcattgccc |
| | ctccccattgacgtcaatggggatgtacttggcagccatcgcgggccatttaccgc |
| | cattgacgtcaatgggagtactgccaatgtaccctggcgtacttccaatagtaatg |
| | tacttgccaagttactattaatagatattgatgtactgccaagtgggccatttacc |
| | gtcattgacgtcaatagggggcgtgagaacggatatgaatgggcaatgagccatcc |
| | cattgacgtcaatggtgggtggtcctattgacgtcaatgggcattgagccaggcgg |
| | gccatttaccgtaattgacgtcaatgggggaggcgccatatacgtcaataggaccg |
| | cccatatgacgtcaataggaaagaccatgctaagccgaattatcgcggctatctga |
| | ggggactagggtgtgtttaggcgaaaagcggggcttcggttgtacgcggttaggag |
| | tcccctcaggatatagtagtttcgcttttgcataggggggggaaatgtagtctta |
| | tgcaatactcttgtagtcttgcaacatggtaacgatgagttagcaacatgccttac |
| | aaggagagaaaaagcaccgtgcatgccgattggtggaagtaaggtggtacgatcgt |
| | gccttattaggaaggcaacagacgggtctgacatggattggacgaaccactgaatt |
| | ccgcattgcagagatattgtatttaagtgcctagctcgatacaataaacgccattt |
| | gaccattcaccacattggtgtgcacctccaagcttcgaccaattctcatgtttgac |
| | agcttatcatcgcagatccgggcaacgttgttgccattgctgcaggcgcagaactg |
| | gtaggtatggaagatctatacattgaatcaatattggcaattagccatattagtca |
| | ttggttatatagcataaatcaatattggctattggccattgcatacgttgtatcta |
| | tatcataatatgtacatttatattggctcatgtccaatatgaccgccatgttgaca |
| | ttgattattgactagttattaatagtaatcaattacggggtcattagttcatagcc |
| | catatatggagttccgcgttacataacttacggtaaatggcccgcctggctgaccg |
| | cccaacgacccccgcccattgacgtcaataatgacgtatgttcccatagtaacgcc |
| | aatagggactttccattgacgtcaatgggtggagtatttacggtaaactgcccact |
| | tggcagtacatcaagtgtatcatatgccaagtccgccccctattgacgtcaatgac |
| | ggtaaatggcccgcctggcattatgcccagtacatgaccttacgggactttcctac |
| | ttggcagtacatctacgtattagtcatcgctattaccatggtgatgcggttttggc |
| | agtacatcaatgggcgtggatagcggtttgactcacggggatttccaagtctccac |
| | cccattgacgtcaatgggagtttgttttggcaccaaaatcaacgggactttccaaa |
| | atgtcgtaacaactccgccccattgacgcaaatgggcggtaggcgtgtacggtggg |
| | aggtctatataagcagagctcgtttagtgaaccgtcagatcgcctggagacgccat |
| | ccacgctgttttgacctccatagaagacaccgggaccgatccagcctccgcgggcg |
| | cgcgtcgaccaccatgtgtccagcgcgcagcctcctccttgtggctaccctggtcc |
| | tcctggaccacctcagtttggccagaaacctccccgtggccactccagacccagga |
| | atgttcccatgccttcaccactcccaaaacctgctgagggccgtcagcaacatgct |
| | ccagaaggccagacaaactctagaattttacccttgcacttctgaagagattgatc |
| | atgaagatatcacaaaagataaaaccagcacagtggaggcctgtttaccattggaa |
| | ttaaccaagaatgagagttgcctaaattccagagagacctctttcataactaatgg |
| | gagttgcctggcctccagaaagacctcttttatgatggccctgtgccttagtagta |
| | tttatgaagacttgaagatgtaccaggtggagttcaagaccatgaatgcaaagctt |
| | ctgatggatcctaagaggcagatctttctagatcaaaacatgctggcagttattga |
| | tgagctgatgcaggccctgaatttcaacagtgagactgtgccacaaaaatcctccc |
| | ttgaagaaccggatttttataaaactaaaatcaagctctgcatacttcttcatgct |
| | ttcagaattcgggcagtgactattgatagagtgatgagctatctgaatgcttccta |
| | atagacgcgtaaaaagatccagacatgataagatacattgatgagtttggacaaac |
| | cacaactagaatgcagtgaaaaaaatgctttatttgtgaaatttgtgatgctattg |
| | ctttatttgtaaccattataagctgcaataaacaagttaacaacaacaattgcatt |
| | cattttatgtttcaggttcagggggaggtgtgggaggtttttt |

The disclosure also relates to cells comprising any one or plurality of nucleic acid sequences disclosed herein. In some embodiments, a cell comprises one or a plurality of nucleic acid sequences disclosed herein. In some embodiments, a host cell comprises a plasmid disclosed herein or comprises any one or plurality of plasmids encoding from about 1 to about 100 tumor-specific antigens, wherein at least one of the tumor-specific antigen is an amino acid sequence associated with the WNT pathway. A host cell can be transfected in vivo (i.e., in an animal) or ex vivo (i.e., outside of an animal). Transfection of a nucleic acid molecule into a host cell can be accomplished by any method by which a nucleic acid molecule can be inserted into the cell. Transfection techniques include, but are not limited to, transfection, electroporation, microinjection, lipofection, adsorption, and protoplast fusion.

The disclosure also provides that the one or more neo-antigenic peptides of the disclosure may be encoded by a single expression vector. The disclosure also provides that the one or more neo-antigenic peptides of the disclosure may be encoded and expressed in vivo using a viral based system (e.g., an adenovirus system).

The term “polynucleotide encoding a polypeptide” encompasses a polynucleotide which includes only coding sequences for the polypeptide as well as a polynucleotide which includes additional coding and/or non-coding sequences. The polynucleotides of the disclosure can be in the form of RNA or in the form of DNA. DNA includes cDNA, genomic DNA, and synthetic DNA; and can be double-stranded or single-stranded, and if single stranded can be the coding strand or non-coding (anti-sense) strand.

In some embodiments, the polynucleotides may comprise the coding sequence for the tumor specific neo-antigenic peptide fused in the same reading frame to a polynucleotide which aids, for example, in expression and/or secretion of a polypeptide from a host cell (e.g., a leader sequence which functions as a secretory sequence for controlling transport of a polypeptide from the cell). The polypeptide having a leader sequence is a preprotein and can have the leader sequence cleaved by the host cell to form the mature form of the polypeptide.

In some embodiments, the polynucleotides can comprise the coding sequence for the tumor specific neo-antigenic peptide fused in the same reading frame to a marker sequence that allows, for example, for purification of the encoded polypeptide, which may then be incorporated into the personalized neoplasia vaccine. For example, the marker sequence can be a hexa-histidine tag (SEQ ID NO: 367) supplied by a pQE-9 vector to provide for purification of the mature polypeptide fused to the marker in the case of a bacterial host, or the marker sequence can be a hemagglutinin (HA) tag derived from the influenza hemagglutinin protein when a mammalian host (e.g., COS-7 cells) is used. Additional tags include, but are not limited to, Calmodulin tags, FLAG tags, Myc tags, S tags, SBP tags, Softag 1, Softag 3, V5 tag, Xpress tag, Isopeptag, SpyTag, Biotin Carboxyl Carrier Protein (BCCP) tags, GST tags, fluorescent protein tags (e.g., green fluorescent protein tags), maltose binding protein tags, Nus tags, Strep-tag, thioredoxin tag, TC tag, Ty tag, and the like.

In embodiments, the polynucleotides may comprise the coding sequence for one or more of the tumor specific neo-antigenic peptides fused in the same reading frame to create a single concatamerized neo-antigenic peptide construct capable of producing multiple neo-antigenic peptides.

In embodiments, the present disclosure provides isolated nucleic acid molecules having a nucleotide sequence at least 60% identical, at least 65% identical, at least 70% identical, at least 75% identical, at least 80% identical, at least 85% identical, at least 90% identical, at least 95% identical, or at least 96%, 97%, 98% or 99% identical to a polynucleotide encoding a tumor specific neoantigen of the present disclosure.

By a polynucleotide having a nucleotide sequence at least, for example, 95% “identical” to a reference nucleotide sequence is intended that the nucleotide sequence of the polynucleotide is identical to the reference sequence except that the polynucleotide sequence can include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence can be deleted or substituted with another nucleotide, or a number of nucleotides up to 5% of the total nucleotides in the reference sequence can be inserted into the reference sequence. These mutations of the reference sequence can occur at the amino- or carboxy-terminal positions of the reference nucleotide sequence or anywhere between those terminal positions, interspersed either individually among nucleotides in the reference sequence or in one or more contiguous groups within the reference sequence.

As a practical matter, whether any particular nucleic acid molecule is at least 80% identical, at least 85% identical, at least 90% identical, and in some embodiments, at least 95%, 96%, 97%, 98%, or 99% identical to a reference sequence can be determined conventionally using known computer programs such as the Bestfit program (Wisconsin Sequence Analysis Package, Version 8 for Unix, Genetics Computer Group, University Research Park, 575 Science Drive, Madison, Wis. 53711). Bestfit uses the local homology algorithm of Smith and Waterman, Advances in Applied Mathematics 2:482-489 (1981), to find the best segment of homology between two sequences. When using Bestfit or any other sequence alignment program to determine whether a particular sequence is, for instance, 95% identical to a reference sequence according to the present disclosure, the parameters are set such that the percentage of identity is calculated over the full length of the reference nucleotide sequence and that gaps in homology of up to 5% of the total number of nucleotides in the reference sequence are allowed.

The present disclosure also includes a composition comprising one or a plurality of nucleic acid molecules described herein.

The present disclosure also contemplates the use of nucleic acid molecules as vehicles for delivering neo-antigens to the subject in vivo in the form of, e.g., DNA/RNA vaccines (see, e.g., WO2012/159643, and WO2012/159754, hereby incorporated by reference in their entirety).

In some embodiments, the personalized neoplasia vaccine may include separate DNA plasmids encoding, for example, one or more neo-antigenic peptides/polypeptides as identified in according to the disclosure. As discussed above, the exact choice of expression vectors will depend upon the peptide/polypeptides to be expressed, and is well within the skill of the ordinary artisan. The expected persistence of the DNA constructs (e.g., in an episomal, non-replicating, non-integrated form in the muscle cells) is expected to provide an increased duration of protection. In some embodiments, the composition comprises a first, second or third nucleic acid molecule, wherein at least the first nucleic acid molecule encodes one or more neoantigens. In some embodiments, the second nucleic acid molecule comprises one or more neoantigens. In some embodiments, the second nucleic acid molecule comprising a nucleic acid sequence that encodes one or more adjuvants. In other embodiments, the personalized neoplasia vaccine may include separate RNA or cDNA molecules encoding neo-antigenic peptides/polypeptides of the disclosure.

In some embodiments the personalized neoplasia or cancer vaccine may include a viral-based vector for use in a human patient such as, for example, and adenovirus system (see, e.g., Baden et al. First-in-human evaluation of the safety and immunogenicity of a recombinant adenovirus serotype 26 HIV-1 Env vaccine (IPCAVD 001). J Infect Dis. 2013 Jan. 15; 207 (2): 240-7, hereby incorporated by reference in its entirety).

### Methods of Identifying Neoantigens

As described in more detail herein, a population of neoplasia/tumor specific neoantigens may be identified by sequencing the neoplasia/tumor and normal DNA of each patient to identify tumor-specific mutations, and determining the patient's HLA allotype. The population of neoplasia/tumor specific neo-antigens and their cognate native antigens may be subject to bioinformatic analysis using validated algorithms to predict which tumor-specific mutations create epitopes that could bind to the patient's HLA allotype, and in particular which tumor-specific mutations create epitopes that could bind to the patient's HLA allotype more effectively than the cognate native antigen. Based on this analysis, identified nucleotide sequences corresponding to these mutations may be designed for each patient, and used together for use as a cancer vaccine in immunizing the subject in need thereof.

The disclosure features a method of identifying one or more subject-specific neoantigen mutations in a subject, wherein the subject has been diagnosed with, suspected of having or comprises one or more hyperproliferative cells (e.g. such as a tumor). In some embodiments, the disclosure features a method of identifying one or more subject-specific neoantigen mutations in a subject, wherein the subject has been diagnosed with, suspected of having or comprises one or more hyperproliferative cells (e.g. such as a tumor) characterized by the presence or quantity of a plurality of neoantigen mutations, the method comprising sequencing a nucleic acid sample from a tumor of the subject and of a non-tumor sample of the subject; analyzing the sequence to determine coding and non-coding regions; identifying sequences comprising tumor-specific non-synonymous or non-silent mutations not present in the non-tumor sample; identifying single nucleotide variations and single nucleotide insertions and deletions; producing subject-specific peptides encoded by the sequences comprising tumor-specific non-synonymous or non-silent mutations not present in the non-tumor sample; and measuring the binding characteristics of the of the subject-specific peptides, wherein each subject-specific peptide is an expression product of subject-specific DNA neoantigen not present in the non-tumor sample, thereby identifying one or more subject-specific DNA neoantigens in a subject. In some embodiments, measuring the binding characteristics of the of the subject-specific peptides is carried out by one or more of measuring the binding of the subject-specific peptides to T-cell receptor; measuring the binding of the subject-specific peptides to a HLA protein of the subject; or measuring the binding of the subject-specific peptides to transporter associated with antigen processing (TAP).

Efficiently choosing which particular mutations to utilize as immunogen requires identification of the patient HLA type and the ability to predict which mutated peptides would efficiently bind to the patient's HLA alleles. Therefore, in some embodiments, measuring the binding of the subject-specific peptides to T-cell receptor comprises measuring the binding of the subject-specific peptides to a HLA protein of the subject or sample.

In some embodiments, the subject-specific peptides bind to HLA proteins of the subject with an IC50 of less than about 550 nM. In some embodiments, the subject-specific peptides bind to HLA proteins of the subject with an IC50 of less than about 500 nM. In some embodiments, the subject-specific peptides bind to HLA proteins of the subject with an IC50 of less than about 450 nM. In some embodiments, the subject-specific peptides bind to HLA proteins of the subject with an IC50 of less than about 400 nM. In some embodiments, the subject-specific peptides bind to HLA proteins of the subject with an IC50 of less than about 350 nM. In some embodiments, the subject-specific peptides bind to HLA proteins of the subject with an IC50 of less than about 300 nM.

In another embodiment, the method of identifying one or more subject-specific DNA neoantigen mutations in a subject further comprises the step of ranking the subject-specific peptides based on the binding characteristics.

In another embodiment, the method of identifying one or more subject-specific DNA neoantigen mutations in a subject, further comprises the step of measuring the CD8+ T cell immune response generated by the subject-specific peptides. Methods of measuring the CD8+ T cell response are known in the art and described herein.

In a further embodiment, the method of identifying one or more subject-specific DNA neoantigen mutations in a subject further comprises formulating the subject-specific DNA neoantigens into an immunogenic composition for administration to the subject. In some embodiments, the top 200 ranked neo-antigen mutations are included or subcloned into the immunogenic composition, which in some embodiments, is one or a plurality of plasmids. In another embodiment, the top 150 ranked neo-antigen mutations are included in the immunogenic composition. In another embodiment, the top 100 ranked neo-antigen mutations are included in the immunogenic composition. In another embodiment, the top 50 ranked neo-antigen mutations are included in the immunogenic composition. In another embodiment, the top 25 ranked neo-antigen mutations are included in the immunogenic composition. In another embodiment, the top 10 ranked neo-antigen mutations are included in the immunogenic composition. In another embodiment, the top 5 ranked neo-antigen mutations are included in the immunogenic composition. In another embodiment, the top 5-20 ranked neo-antigen mutations are included in the immunogenic composition. In another embodiment, the top 10-50 ranked neo-antigen mutations are included in the immunogenic composition. In another embodiment, the top 25-100 ranked neo-antigen mutations are included in the immunogenic composition. In another embodiment, the top 50-100 ranked neo-antigen mutations are included in the immunogenic composition. In another embodiment, the top 100-200 ranked neo-antigen mutations are included in the immunogenic composition.

In another embodiment, the method of identifying one or more subject-specific DNA neoantigen mutations in a subject further comprises providing a culture comprising dendritic cells (DCs) obtained from the subject; and contacting the dendritic cells with the immunogenic composition. DCs are potent antigen-presenting cells that initiate T cell immunity and can be used as cancer vaccines when loaded with one or more neoantigens of interest. In a further embodiment, the method further comprises administering to the subject the dendritic cells; obtaining a population of CD8+ T cells from a peripheral blood sample from the subject, wherein the CD8+ cells recognize the at least one neoantigen; and expanding the population of CD8+ T cells that recognizes the neoantigen.

In some embodiments, the expanded population of CD8+ T cells is administered to the subject.

Preferably, any suitable sequencing-by-synthesis platform can be used to identify mutations. Four major sequencing-by-synthesis platforms are currently available: the Genome Sequencers from Roche/454 Life Sciences, the HiSeq Analyzer from Illumina/Solexa, the SOLID system from Applied BioSystems, and the Heliscope system from Helicos Biosciences. Sequencing-by-synthesis platforms have also been described by Pacific Biosciences and VisiGen Biotechnologies. Each of these platforms can be used in the methods of the disclosure. In some embodiments, a plurality of nucleic acid molecules being sequenced is bound to a support (e.g., solid support). To immobilize the nucleic acid on a support, a capture sequence/universal priming site can be added at the 3′ and/or 5′ end of the template. The nucleic acids may be bound to the support by hybridizing the capture sequence to a complementary sequence covalently attached to the support. The capture sequence (also referred to as a universal capture sequence) is a nucleic acid sequence complementary to a sequence attached to a support that may dually serve as a universal primer.

As an alternative to a capture sequence, a member of a coupling pair (such as, e.g., antibody/antigen, receptor/ligand, or the avidin-biotin pair as described in, e.g., U.S. Patent Application No. 2006/0252077) may be linked to each fragment to be captured on a surface coated with a respective second member of that coupling pair. Subsequent to the capture, the sequence may be analyzed, for example, by single molecule detection/sequencing, e.g., as described in the Examples and in U.S. Pat. No. 7,283,337, including template-dependent sequencing-by-synthesis. In sequencing-by-synthesis, the surface-bound molecule is exposed to a plurality of labeled nucleotide triphosphates in the presence of polymerase. The sequence of the template is determined by the order of labeled nucleotides incorporated into the 3′ end of the growing chain. This can be done in real time or in a step-and-repeat mode. For real-time analysis, different optical labels to each nucleotide may be incorporated and multiple lasers may be utilized for stimulation of incorporated nucleotides.

Any cell type or tissue may be utilized to obtain nucleic acid samples for use in the sequencing methods described herein. In some embodiments, the DNA or RNA sample is obtained from a neoplasia, a tumor or a bodily fluid, e.g., blood, obtained by known techniques (e.g. venipuncture) or saliva. Alternatively, nucleic acid tests can be performed on dry samples (e.g. hair or skin).

A variety of methods are available for detecting the presence of a particular mutation or allele in an individual's DNA or RNA. Advancements in this field have provided accurate, easy, and inexpensive large-scale SNP genotyping. Most recently, for example, several new techniques have been described including dynamic allele-specific hybridization (DASH), microplate array diagonal gel electrophoresis (MADGE), pyrosequencing, oligonucleotide-specific ligation, the TaqMan system as well as various DNA “chip” technologies such as the Affymetrix SNP chips. These methods require amplification of the target genetic region, typically by PCR. Still other newly developed methods, based on the generation of small signal molecules by invasive cleavage followed by mass spectrometry or immobilized padlock probes and rolling-circle amplification, might eventually eliminate the need for PCR. Several of the methods known in the art for detecting specific single nucleotide polymorphisms are summarized below. The method of the present disclosure is understood to include all available methods.

PCR based detection means may include multiplex amplification of a plurality of markers simultaneously. For example, it is well known in the art to select PCR primers to generate PCR products that do not overlap in size and can be analyzed simultaneously.

Alternatively, it is possible to amplify different markers with primers that are differentially labeled and thus can each be differentially detected. Of course, hybridization based detection means allow the differential detection of multiple PCR products in a sample. Other techniques are known in the art to allow multiplex analyses of a plurality of markers.

Several methods have been developed to facilitate analysis of single nucleotide polymorphisms in genomic DNA or cellular RNA. In some embodiments, the single base polymorphism can be detected by using a specialized exonuclease-resistant nucleotide, as disclosed, e.g., U.S. Pat. No. 4,656,127. According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data.

In another embodiment of the disclosure, a solution-based method is used for determining the identity of the nucleotide of a polymorphic site. Cohen et al. (French Patent No. 2,650,840; PCT Application No. WO1991/02087). As in the method of U.S. Pat. No. 4,656,127, a primer may be employed that is complementary to allelic sequences immediately 3′ to a polymorphic site. The method determines the identity of the nucleotide of that site using labeled dideoxynucleotide derivatives, which, if complementary to the nucleotide of the polymorphic site, will become incorporated onto the terminus of the primer.

An alternative method, known as Genetic Bit Analysis or GBA is described in PCT Application No. WO1992/15712). GBA uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Application No. WO1991/02087) the GBA method is preferably a heterogeneous phase assay, in which the primer or the target molecule is immobilized to a solid phase.

Several primer-guided nucleotide incorporation procedures for assaying polymorphic sites in DNA have been described (Komher, J. S. et al., Nucl. Acids. Res. 17:7779-7784 (1989); Sokolov, B. P., Nucl. Acids Res. 18:3671 (1990); Syvanen, A.-C, et al., Genomics 8:684-692 (1990); Kuppuswamy, M. N. et al., Proc. Natl. Acad. Sci. (U.S.A.) 88:1143-1147 (1991); Prezant, T. R. et al., Hum. Mutat. 1:159-164 (1992); Ugozzoli, L. et al., GATA 9:107-112 (1992); Nyren, P. et al., Anal. Biochem. 208:171-175 (1993)). These methods differ from GBA in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A.-C, et al., Amer. J. Hum. Genet. 52:46-59 (1993)).

The disclosure generally relates to a method of identifying or selecting one or a plurality of neoantigens from a sample, the method comprising (a) sequencing the DNA/RNA from a sample, and (b) measuring the binding of the subject-specific peptides to T-cell receptor comprises measuring the binding of the subject-specific peptides to a HLA protein of the subject or sample, and (c) selecting or a plurality of neoantigens from a sample if the HLA protein from the subject binds to HLA proteins of the subject with an IC50 of less than about 500 nM, 400 nM, 300 nM, 200 nM, or 100 nM; and, optionally (d) ordering the neoantigens in order of lowest IC50 value to highest IC50 value.

In some embodiments, the disclosure relates to generating a vaccine or manufacturing a pharmaceutical composition comprising performing any one or more of the aforementioned steps and further comprising subcloning a nucleic acid sequence encoding the one or plurality of neoantigens into one or more nucleic acid molecules; and, optionally, suspending the nucleic acid molecules in one or more pharmaceutically acceptable carriers.

In some embodiments, the nucleic acid sequence encoding the neoantigens also comprises a linker. In some embodiments, the nucleic acid molecule is free of a nucleic acid sequence that encodes a P2A linker. In some embodiments, the nucleic acid molecule is free of a nucleic acid sequence that encodes two different linkers. In some embodiments, the nucleic acid molecule is free of a nucleic acid sequence that encodes a linker, such that at least two or a plurality of AED sequences, from the 5′ to 3′ orientation is encoded as a separate polypeptide or as a large contiguous fusion protein.

In another embodiment, the method of identifying one or more subject-specific DNA neoantigen mutations in a subject further comprises providing a culture comprising dendritic cells (DCs) obtained from the subject; and contacting the dendritic cells with the immunogenic composition. DCs are potent antigen-presenting cells that initiate T cell immunity and can be used as cancer vaccines when loaded with one or more neoantigens of interest. In a further embodiment, the method further comprises administering to the subject the dendritic cells; obtaining a population of CD8+ T cells from a peripheral blood sample from the subject, wherein the CD8+ cells recognize the at least one neoantigen; and expanding the population of CD8+ T cells that recognizes the neoantigen.

In some embodiments, the expanded population of CD8+ T cells is administered to the subject.

Preferably, any suitable sequencing-by-synthesis platform can be used to identify mutations. Four major sequencing-by-synthesis platforms are currently available: the Genome Sequencers from Roche/454 Life Sciences, the HiSeq Analyzer from Illumina/Solexa, the SOLiD system from Applied BioSystems, and the Heliscope system from Helicos Biosciences. Sequencing-by-synthesis platforms have also been described by Pacific Biosciences and VisiGen Biotechnologies. Each of these platforms can be used in the methods of the disclosure. In some embodiments, a plurality of nucleic acid molecules being sequenced is bound to a support (e.g., solid support). To immobilize the nucleic acid on a support, a capture sequence/universal priming site can be added at the 3′ and/or 5′ end of the template. The nucleic acids may be bound to the support by hybridizing the capture sequence to a complementary sequence covalently attached to the support. The capture sequence (also referred to as a universal capture sequence) is a nucleic acid sequence complementary to a sequence attached to a support that may dually serve as a universal primer.

As an alternative to a capture sequence, a member of a coupling pair (such as, e.g., antibody/antigen, receptor/ligand, or the avidin-biotin pair as described in, e.g., U.S. Patent Application No. 2006/0252077) may be linked to each fragment to be captured on a surface coated with a respective second member of that coupling pair. Subsequent to the capture, the sequence may be analyzed, for example, by single molecule detection/sequencing, e.g., as described in the Examples and in U.S. Pat. No. 7,283,337, including template-dependent sequencing-by-synthesis. In sequencing-by-synthesis, the surface-bound molecule is exposed to a plurality of labeled nucleotide triphosphates in the presence of polymerase. The sequence of the template is determined by the order of labeled nucleotides incorporated into the 3′ end of the growing chain. This can be done in real time or in a step-and-repeat mode. For real-time analysis, different optical labels to each nucleotide may be incorporated and multiple lasers may be utilized for stimulation of incorporated nucleotides.

Any cell type or tissue may be utilized to obtain nucleic acid samples for use in the sequencing methods described herein. In some embodiments, the DNA or RNA sample is obtained from a neoplasia/tumor or a bodily fluid, e.g., blood, obtained by known techniques (e.g. venipuncture) or saliva. Alternatively, nucleic acid tests can be performed on dry samples (e.g. hair or skin).

A variety of methods are available for detecting the presence of a particular mutation or allele in an individual's DNA or RNA. Advancements in this field have provided accurate, easy, and inexpensive large-scale SNP genotyping. Most recently, for example, several new techniques have been described including dynamic allele-specific hybridization (DASH), microplate array diagonal gel electrophoresis (MADGE), pyrosequencing, oligonucleotide-specific ligation, the TaqMan system as well as various DNA “chip” technologies such as the Affymetrix SNP chips. These methods require amplification of the target genetic region, typically by PCR. Still other newly developed methods, based on the generation of small signal molecules by invasive cleavage followed by mass spectrometry or immobilized padlock probes and rolling-circle amplification, might eventually eliminate the need for PCR. Several of the methods known in the art for detecting specific single nucleotide polymorphisms are summarized below. The method of the present disclosure is understood to include all available methods.

PCR based detection means may include multiplex amplification of a plurality of markers simultaneously. For example, it is well known in the art to select PCR primers to generate PCR products that do not overlap in size and can be analyzed simultaneously.

Alternatively, it is possible to amplify different markers with primers that are differentially labeled and thus can each be differentially detected. Of course, hybridization-based detection means allow the differential detection of multiple PCR products in a sample. Other techniques are known in the art to allow multiplex analyses of a plurality of markers.

Several methods have been developed to facilitate analysis of single nucleotide polymorphisms in genomic DNA or cellular RNA. In some embodiments, the single base polymorphism can be detected by using a specialized exonuclease-resistant nucleotide, as disclosed, e.g., U.S. Pat. No. 4,656,127. According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data.

In another embodiment of the disclosure, a solution-based method is used for determining the identity of the nucleotide of a polymorphic site. Cohen et al. (French Patent No. 2,650,840; PCT Application No. WO1991/02087). As in the method of U.S. Pat. No. 4,656,127, a primer may be employed that is complementary to allelic sequences immediately 3′ to a polymorphic site. The method determines the identity of the nucleotide of that site using labeled dideoxynucleotide derivatives, which, if complementary to the nucleotide of the polymorphic site, will become incorporated onto the terminus of the primer.

An alternative method, known as Genetic Bit Analysis or GBA is described in PCT Application No. WO1992/15712). GBA uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Application No. WO1991/02087) the GBA method is preferably a heterogeneous phase assay, in which the primer or the target molecule is immobilized to a solid phase.

Several primer-guided nucleotide incorporation procedures for assaying polymorphic sites in DNA have been described (Komher, J. S. et al., Nucl. Acids. Res. 17:7779-7784 (1989); Sokolov, B. P., Nucl. Acids Res. 18:3671 (1990); Syvanen, A.-C, et al., Genomics 8:684-692 (1990); Kuppuswamy, M. N. et al., Proc. Natl. Acad. Sci. (U.S.A.) 88:1143-1147 (1991); Prezant, T. R. et al., Hum. Mutat. 1:159-164 (1992); Ugozzoli, L. et al., GATA 9:107-112 (1992); Nyren, P. et al., Anal. Biochem. 208:171-175 (1993)). These methods differ from GBA in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A.-C, et al., Amer. J. Hum. Genet. 52:46-59 (1993)).

### Pharmaceutical Compositions

The terms “pharmaceutical preparation” or “pharmaceutical composition” includes preparations suitable for administration to mammals, e.g., humans. When the compounds of the present disclosure are administered as pharmaceuticals to mammals, e.g., humans, they can be given per se or as a pharmaceutical composition containing, for example, from about 0.1 to about 99.5% of active ingredient in combination with a pharmaceutically acceptable carrier. In some embodiments, the pharmaceutical composition comprises a nucleic acid molecule comprising the expressible nucleic acid sequence disclosed herein; and a pharmaceutically acceptable carrier comprising a saline buffer. In some embodiments, the saline buffer is saline sodium citrate buffer.

The phrase “pharmaceutically acceptable” refers to molecular entities and compositions that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans.

The phrase “pharmaceutically acceptable carrier” is art recognized and includes a pharmaceutically acceptable material, composition or vehicle, suitable for administering compounds of the present disclosure to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Suitable pharmaceutical carriers are described in “Remington's Pharmaceutical Sciences” by E. W. Martin, which is incorporated herein by reference in its entirety.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, α-tocopherol, and the like; and metal chelating agents, such as citric acid, ethylenediamine tetraacetic acid (EDTA), sorbitol, tartaric acid, phosphoric acid, and the like.

Formulations of the present disclosure include those suitable for oral, nasal, intravenous, intraperitoneal, intramuscular, intratopical, buccal, sublingual, rectal, vaginal and/or parenteral administration. The formulations may conveniently be presented in unit dosage form and may be prepared by any methods well known in the art of pharmacy. The amount of active ingredient that can be combined with a carrier material to produce a single dosage form will generally be that amount of the compound that produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a nucleic acid sequence or composition of the present disclosure with the carrier and, optionally, one or more accessory ingredients. In general, the formulations are prepared by uniformly and intimately bringing into association a compound of the present disclosure with liquid carriers, or finely divided solid carriers, or both, and then, if necessary, shaping the product.

Formulations of the disclosure suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present disclosure as an active ingredient. A compound of the present disclosure may also be administered as a bolus, electuary or paste.

In solid dosage forms for oral administration (capsules, tablets, pills, dragees, powders, granules and the like), the active ingredient is mixed with one or more pharmaceutically acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol and glycerol monostearate; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hardfilled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present disclosure, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes, catanionic vesicles, and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms for oral administration of the compounds of the present disclosure include pharmaceutically acceptable emulsions, microemulsions, solutions, suspensions, syrups and elixirs. In addition to the active ingredient, the liquid dosage forms may contain inert diluent commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents. Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions for rectal or vaginal administration may be presented as a suppository, which may be prepared by mixing one or more compounds of the disclosure with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound. Formulations of the present disclosure which are suitable for vaginal administration also include pessaries, tampons, creams, gels, pastes, foams or spray formulations containing such carriers as are known in the art to be appropriate. Dosage forms for the topical or transdermal administration of a compound of this disclosure include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present disclosure to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the active compound in a polymer matrix or gel.

Ophthalmic formulations, eye ointments, powders, solutions and the like, are also contemplated as being within the scope of this disclosure.

Liquid dosage forms for parenteral administration include, but are not limited to, pharmaceutically acceptable emulsions, microemulsions, nanoemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include additional therapeutics and/or prophylactics, additional agents such as wetting agents, emulsifying and suspending agents, sweetening, flavoring, and/or perfuming agents. In certain embodiments for parenteral administration, compositions are mixed with solubilizing agents such as Cremophor®, alcohols, oils, modified oils, glycols, polysorbates, cyclodextrins, polymers, and/or combinations thereof.

Pharmaceutical compositions suitable for parenteral administration comprise one or more compounds of the disclosure in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables.

Injectable formulations can be sterilized, for example, by filtration through a bacterial retaining filter, and/or by incorporating sterilizing agents in the form of sterile solid compositions which can be dissolved or dispersed in sterile water or other sterile injectable medium prior to use.

Examples of suitable aqueous and nonaqueous carriers that may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue. The preparations may be given orally, parenterally, topically, or rectally. They can by forms suitable for each administration route. For example, they are administered in tablets or capsule form, by injection, inhalation, eye lotion, ointment, suppository, etc., administration by injection, infusion or inhalation; topical by lotion or ointment; and rectal by suppositories. Oral and/or IV administration is preferred.

The terms “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection, and infusion.

The terms “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present disclosure, which may be used in a suitable hydrated form, and/or the pharmaceutical compositions of the present disclosure, are formulated into pharmaceutically acceptable dosage forms by conventional methods known to those of skill in the art.

Actual dosage levels of the active ingredients in the pharmaceutical compositions of this disclosure may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present disclosure employed, or the ester, salt or amide thereof, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the disclosure employed in the pharmaceutical composition at levels lower than that required in order to achieve the desired therapeutic effect and gradually increase the dosage until the desired effect is achieved.

In general, a suitable daily dose of a compound of the disclosure will be that amount of the compound that is the lowest dose effective to produce a therapeutic effect. Such an effective dose will generally depend upon the factors described above. Generally, subcutaneous doses of the compounds of this disclosure for a patient, when used for the indicated anti-tumor effects, will range from about 0.0001 to about 100 mg, from about 0.01 to about 50 mg, from about 0.1 to about 5 mg, from about 0.2 to about 5 mg and from about 0.3 to about 3 mg per dose. An effective amount is an amount that treats a Wnt signaling-related disorder.

If desired, the effective daily dose of the active compound may be administered as two, three, four, five, six or more sub-doses administered separately at appropriate intervals throughout the year, optionally, in unit dosage forms. While it is possible for a compound of the present disclosure to be administered alone, it is preferable to administer the compound as a pharmaceutical composition in any of the methods disclosed herein.

Compounds of the present disclosure are prepared from commonly available compounds using procedures known to those skilled in the art, including any one or more of the disclosed conditions without limitation:

Within the scope of this text, only a readily removable group that is not a constituent of the particular desired end product of the compounds of the present disclosure is designated a “protecting group,” unless the context indicates otherwise. The protection of functional groups by such protecting groups, the protecting groups themselves, and their cleavage reactions are described for example in standard reference works, such as e.g., Science of Synthesis: Houben-Weyl Methods of Molecular Transformation. Georg Thieme Verlag, Stuttgart, Germany. 2005. 41627 pp. (URL: http://www.science-of-synthesis.com (Electronic Version, 48 Volumes)); J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie” (Methods of Organic Chemistry), Houben Weyl, 4th edition, Volume 15/I, Georg Thieme Verlag, Stuttgart 1974, in H.-D. Jakubke and H. Jeschkeit, “Aminosauren, Peptide, Proteine” (Amino acids, Peptides, Proteins), Verlag Chemie, Weinheim, Deerfield Beach, and Basel 1982, and in Jochen Lehmann, “Chemie der Kohlenhydrate: Monosaccharide and Derivate” (Chemistry of Carbohydrates: Monosaccharides and Derivatives), Georg Thieme Verlag, Stuttgart 1974. A characteristic of protecting groups is that they can be removed readily (i.e., without the occurrence of undesired secondary reactions) for example by solvolysis, reduction, photolysis or alternatively under physiological conditions (e.g., by enzymatic cleavage).

Acid addition salts of the disclosed compounds are most suitably formed from pharmaceutically acceptable acids, and include for example those formed with inorganic acids e.g. hydrochloric, hydrobromic, sulphuric or phosphoric acids and organic acids e.g. succinic, malaeic, acetic or fumaric acid. Other non-pharmaceutically acceptable salts e.g. oxalates can be used for example in the isolation of the disclosed compounds, for laboratory use, or for subsequent conversion to a pharmaceutically acceptable acid addition salt. Also included within the scope of the disclosure are solvates and hydrates.

In vivo hydrolyzable esters or amides of certain compounds of the disclosure can be formed by treating those compounds having a free hydroxy or amino functionality with the acid chloride of the desired ester in the presence of a base in an inert solvent such as methylene chloride or chloroform. Suitable bases include triethylamine or pyridine. Conversely, compounds having a free carboxy group can be esterified using standard conditions which can include activation followed by treatment with the desired alcohol in the presence of a suitable base.

Examples of pharmaceutically acceptable addition salts include, without limitation, the non-toxic inorganic and organic acid addition salts such as the hydrochloride derived from hydrochloric acid, the hydrobromide derived from hydrobromic acid, the nitrate derived from nitric acid, the perchlorate derived from perchloric acid, the phosphate derived from phosphoric acid, the sulphate derived from sulphuric acid, the formate derived from formic acid, the acetate derived from acetic acid, the aconate derived from aconitic acid, the ascorbate derived from ascorbic acid, the benzenesulphonate derived from benzensulphonic acid, the benzoate derived from benzoic acid, the cinnamate derived from cinnamic acid, the citrate derived from citric acid, the embonate derived from embonic acid, the enantate derived from enanthic acid, the fumarate derived from fumaric acid, the glutamate derived from glutamic acid, the glycolate derived from glycolic acid, the lactate derived from lactic acid, the maleate derived from maleic acid, the malonate derived from malonic acid, the mandelate derived from mandelic acid, the methanesulphonate derived from methane sulphonic acid, the naphthalene-2-sulphonate derived from naphtalene-2-sulphonic acid, the phthalate derived from phthalic acid, the salicylate derived from salicylic acid, the sorbate derived from sorbic acid, the stearate derived from stearic acid, the succinate derived from succinic acid, the tartrate derived from tartaric acid, the toluene-p-sulphonate derived from p-toluene sulphonic acid, and the like. In some embodiments, the salts are sodium, lysine and arginine salts of the compounds of the disclosure. Such salts can be formed by procedures well known and described in the art.

Other acids such as oxalic acid, which cannot be considered pharmaceutically acceptable, can be useful in the preparation of salts useful as intermediates in obtaining a chemical compound of the disclosure and its pharmaceutically acceptable acid addition salt. Metal salts of a chemical compound of the disclosure include alkali metal salts, such as the sodium salt of a chemical compound of the disclosure containing a carboxy group.

Mixtures of isomers obtainable according to the disclosure can be separated in a manner known per se into the individual isomers; diastereoisomers can be separated, for example, by partitioning between polyphasic solvent mixtures, recrystallisation and/or chromatographic separation, for example over silica gel or by, e.g., medium pressure liquid chromatography over a reversed phase column, and racemates can be separated, for example, by the formation of salts with optically pure salt-forming reagents and separation of the mixture of diastereoisomers so obtainable, for example by means of fractional crystallisation, or by chromatography over optically active column materials.

Intermediates and final products can be worked up and/or purified according to standard methods, e.g., using chromatographic methods, distribution methods, (re-) crystallization, and the like. The following applies in general to all processes mentioned throughout this disclosure.

The compounds, including their salts, may also be obtained in the form of hydrates, or their crystals may, for example, include the solvent used for crystallization. Different crystalline forms may be present.

The disclosure relates also to those forms of the process in which a compound obtainable as an intermediate at any stage of the process is used as starting material and the remaining process steps are carried out, or in which a starting material is formed under the reaction conditions or is used in the form of a derivative, for example in a protected form or in the form of a salt, or a compound obtainable by the process according to the disclosure is produced under the process conditions and processed further in situ.

The disclosure relates to discovering molecular targets that cancer cells rely on to survive, and which may therefore be utilized to kill cancer cells. Cancer cells in tumors display considerable heterogeneity, which arises in part from (epi) genetic differences between cells, but also from variability in their interactions with other cells and the extracellular matrix, as well as access to oxygen, nutrients and endocrine signals. Cellular heterogeneity poses a problem for cancer therapy as subpopulations not dependent on such survival signals are likely to exist, and they may survive therapy and repopulate the tumor (Greaves and Maley, 2012). Cells with such repopulating ability are sometimes termed cancer stem cells (CSCs) (Lobo et al., 2007; Valent et al., 2012); the elimination or re-differentiation of CSCs is an attractive therapeutic strategy. Although such therapy may not be effective on its own, it may allow “homogenization” of cancer cell phenotypes within tumors that may lead to improved treatment responses to conventional cancer therapies.

### Methods of Treating Cancer

The disclosure provides a method of treating and/or preventing cancer in a subject, the method comprising administering to the subject in need thereof a pharmaceutically effective amount of any of the nucleic acid molecules as described herein (e.g. a nucleic acid molecule comprising an expressible nucleic acid sequence comprising Formula I: [[(AEDn)-(linker)]n-[AEDn+1]), any of the pharmaceutical compositions described herein, or any pharmaceutically acceptable salt thereof. In some embodiments, the expressible nucleic acid sequence comprises at least about twenty AED domains, and wherein the expressible nucleic acid sequence is free of a nucleic acid sequence that encodes a WNT pathway tumor-specific antigen. In some embodiments, the expressible nucleic acid sequence comprises at least about twenty AED domains, and wherein the expressible nucleic acid sequence encodes at least one WNT pathway tumor-specific antigen. In some embodiments, the expressible nucleic acid sequence comprises at least about twenty AED domains, and wherein the expressible nucleic acid sequence encodes at least one or a combination of any two or more WNT pathway tumor-specific antigens disclosed herein.

For therapeutic or immunization purposes, nucleic acid molecules of the invention can also be administered to the subject. A number of methods are conveniently used to deliver the nucleic acids to the patient. For instance, the nucleic acid can be delivered directly, as “naked DNA”. This approach is described, for instance, in Wolff et al., Science 247:1465-1468 (1990) as well as U.S. Pat. Nos. 5,580,859 and 5,589,466. The nucleic acids can also be administered using ballistic delivery as described, for instance, in U.S. Pat. No. 5,204,253. Particles comprised solely of DNA can be administered. Alternatively, DNA can be adhered to particles, such as gold particles.

The nucleic acids can also be delivered complexed to cationic compounds, such as cationic lipids. Lipid-mediated gene delivery methods are described, for instance, in WO1996/18372; WO 1993/24640; Mannino & Gould-Fogerite, BioTechniques 6 (7): 682-691 (1988); U.S. Pat. No. 5,279,833; WO 1991/06309; and Feigner et al., Proc. Natl. Acad. Sci. USA 84:7413-7414 (1987).

RNA encoding the peptide of interest can also be used for delivery (see, e.g., Kiken et al, 2011; Su et al, 2011).

In some embodiments, the nucleic acid molecule is administered to the subject by electroporation.

In some embodiments, treatment is determined by a clinical outcome, an increase, enhancement or prolongation of anti-tumor activity by T cells, an increase in the number of anti-tumor T cells or activated T cells as compared with the number prior to treatment, or a combination thereof. In a further embodiment, clinical outcome is selected from the group consisting of tumor regression, tumor shrinkage, tumor necrosis, anti-tumor response by the immune system, tumor expansion, recurrence or spread, or a combination thereof.

Examples of cancers and cancer conditions that can be treated with the combination therapy of this document include, but are not limited to a patient in need thereof that has been diagnosed as having cancer, or at risk of developing cancer. In some embodiments, the subject has previously been treated but no longer responding to checkpoint inhibitor therapy.

The therapy described herein is also applicable where the subject has no detectable neoplasia but is at high risk for disease recurrence.

According to the disclosure, the nucleic acid molecules described herein may be used for a patient that has been diagnosed as having cancer, or at risk of developing cancer.

In some embodiments, the method of treatment further comprises a step of contemporaneously or sequentially administering a nucleic acid sequence encoding a cytokine in addition to the nucleic acid sequence encoding the disclosed plurality of antigens. In some embodiments, the method comprises administering from about 0.5 to about 2 milligrams of nucleic acid encoding a cytokine with from about 0.1 to about 4 milligrams of nucleic acid encoding the one or plurality of antigens. In some embodiments, the nucleic acid sequences are adminstered as plasmids suspended in sterilized sodium phosphate buffer or sterilized sodium citrate buffer.

In some embodiments, the pharmaceutical composition is administered intradermally in two volumes in each of a subject's first and second deltoid regions. In some embodiments, the method further comprises a step of exposing the subject to an electroporation event at one or multiple sites of injection subsequent to the administration of the nucleic acid sequences. Electroporation can be accomplished by exposing the site of injection to a pulse of electricity in a localized fashion. Examples of electroporation devices and electroporation methods preferred for facilitating delivery of the DNA vaccines of the present invention, include those described in U.S. Pat. No. 7,245,963 by Draghia-Akli, et al., U.S. Patent Application Publication No. 2005/0052630 submitted by Smith, et al., the contents of which are hereby incorporated by reference in their entirety. Also preferred, are electroporation devices and electroporation methods for facilitating delivery of the DNA vaccines provided in co-pending and co-owned U.S. patent application Ser. No. 11/874,072, filed Oct. 17, 2007, which claims the benefit under 35 USC 119 (e) to U.S. Provisional Application Ser. No. 60/852,149, filed Oct. 17, 2006, and U.S. Provisional Application Ser. No. 60/978,982, filed Oct. 10, 2007, all of which are hereby incorporated in their entirety.

U.S. Pat. No. 7,245,963 by Draghia-Akli, et al. describes modular electrode systems and their use for facilitating the introduction of a biomolecule into cells of a selected tissue in a body or plant. The modular electrode systems comprise a plurality of needle electrodes; a hypodermic needle; an electrical connector that provides a conductive link from a programmable constant-current pulse controller to the plurality of needle electrodes; and a power source. An operator can grasp the plurality of needle electrodes that are mounted on a support structure and firmly insert them into the selected tissue in a body or plant. The biomolecules are then delivered via the hypodermic needle into the selected tissue. The programmable constant-current pulse controller is activated and constant-current electrical pulse is applied to the plurality of needle electrodes. The applied constant-current electrical pulse facilitates the introduction of the biomolecule into the cell between the plurality of electrodes. The entire content of U.S. Pat. No. 7,245,963 is hereby incorporated by reference.

U.S. Patent Application Publication No. 2005/0052630, incorporated by reference in its entirety herein, describes an electroporation device which may be used to effectively facilitate the introduction of a biomolecule into cells of a selected tissue in a body or plant. The electroporation device comprises an electro-kinetic device (“EKD device”) whose operation is specified by software or firmware. The EKD device produces a series of programmable constant-current pulse patterns between electrodes in an array based on user control and input of the pulse parameters, and allows the storage and acquisition of current waveform data. The electroporation device also comprises a replaceable electrode disk having an array of needle electrodes, a central injection channel for an injection needle, and a removable guide disk.

The electrode arrays and methods described in U.S. Pat. No. 7,245,963 and U.S. Patent Application Publication No. 2005/0052630 are adapted for deep penetration into not only tissues such as muscle, but also other tissues or organs. Because of the configuration of the electrode array, the injection needle (to deliver the biomolecule of choice) is also inserted completely into the target organ, and the injection is administered perpendicular to the target issue, in the area that is pre-delineated by the electrodes. The electrodes described in U.S. Pat. No. 7,245,963 and U.S. Patent Application Publication No. 2005/005263 are preferably 20 mm long and 21 gauge.

In certain exemplary embodiments, electroporation devices can be configured to deliver to a desired tissue of a mammal a pulse of energy producing a constant current similar to a preset current input by a user. The electroporation device comprises an electroporation component and an electrode assembly or handle assembly. The electroporation component can include and incorporate one or more of the various elements of the electroporation devices, including: controller, current waveform generator, impedance tester, waveform logger, input element, status reporting element, communication port, memory component, power source, and power switch. The electroporation component can function as one element of the electroporation devices, and the other elements are separate elements (or components) in communication with the electroporation component. In some embodiments, the electroporation component can function as more than one element of the electroporation devices, which can be in communication with still other elements of the electroporation devices separate from the electroporation component.

The present invention is not limited by the elements of the electroporation devices existing as parts of one electromechanical or mechanical device, as the elements can function as one device or as separate elements in communication with one another. The electroporation component is capable of delivering the pulse of energy that produces the constant current in the desired tissue and includes a feedback mechanism. The electrode assembly includes an electrode array having a plurality of electrodes in a spatial arrangement, wherein the electrode assembly receives the pulse of energy from the electroporation component and delivers same to the desired tissue through the electrodes. At least one of the plurality of electrodes is neutral during delivery of the pulse of energy and measures impedance in the desired tissue and communicates the impedance to the electroporation component. The feedback mechanism can receive the measured impedance and can adjust the pulse of energy delivered by the electroporation component to maintain the constant current.

In some embodiments, the plurality of electrodes can deliver the pulse of energy in a decentralized pattern. In some embodiments, the plurality of electrodes can deliver the pulse of energy in the decentralized pattern through the control of the electrodes under a programmed sequence, and the programmed sequence is input by a user to the electroporation component. In some embodiments, the programmed sequence comprises a plurality of pulses delivered in sequence, wherein each pulse of the plurality of pulses is delivered by at least two active electrodes with one neutral electrode that measures impedance, and wherein a subsequent pulse of the plurality of pulses is delivered by a different one of at least two active electrodes with one neutral electrode that measures impedance.

In some embodiments, the feedback mechanism is performed by either hardware or software. Preferably, the feedback mechanism is performed by an analog closed-loop circuit. In certain embodiments, this feedback occurs every 50 μs, 20 μs, 10 μs or 1 μs, but is preferably a realtime feedback or instantaneous (i.e., substantially instantaneous as determined by available techniques for determining response time). In some embodiments, the neutral electrode measures the impedance in the desired tissue and communicates the impedance to the feedback mechanism, and the feedback mechanism responds to the impedance and adjusts the pulse of energy to maintain the constant current at a value similar to the preset current. In some embodiments, the feedback mechanism maintains the constant current continuously and instantaneously during the delivery of the pulse of energy.

In certain embodiments, the method comprises treating a cancer that is a solid tumor. In some embodiments, the cancer has a high mutational burden (HMB).

Tumor mutational burden is the frequency of certain mutations within a tumor's genes. To be counted toward TMB, mutations must alter a protein expressed by the tumor. Methods of determining a high mutational burden are known in the art and include, for example, next generation whole exome sequencing of tumor tissue, which sequences all of the protein-coding genes within a tumor, and sequencing a gene panel, which provides the sequences of a targeted set of genes. In some embodiments, tumors having a high mutational burden have at least about 10 mutations per million base pairs of tumor DNA.

In other embodiments, the cancer has been shown to have a poor or low response to checkpoint inhibitor therapy. In some embodiments, the subject developed a tolerance to checkpoint inhibitor therapy. Resistance (poor or low response) or tolerance to checkpoint inhibitor therapy is defined as a measured progression of disease in spite of the subject being administered the checkpoint inhibitor therapy. In some embodiments, the subject is resistant to checkpoint inhibitor therapy. In some embodiments, the subject exhibits less than about 2% reduction in tumor mass, no reduction in tumor mass or tumor growth after treatment with checkpoint inhibitors.

In some embodiments, the cancer is adrenocortical cancer. In some embodiments, the cancer is adrenocortical carcinoma. In some embodiments, the cancer is bladder cancer. In some embodiments, the cancer is bladder urothelial carcinoma. In some embodiments, the cancer is breast cancer. In some embodiments, the cancer is cervical adenocarcinoma. In some embodiments, the cancer is cervical squamous cell carcinoma. In some embodiments, the cancer is cholangiocarcinoma. In some embodiments, the cancer is colorectal adenocarcinoma. In some embodiments, the cancer is colorectal cancer. In some embodiments, the cancer is diffuse glioma. In some embodiments, the cancer is endometrial cancer. In some embodiments, the cancer is endometrial carcinoma. In some embodiments, the cancer is esophageal squamous cell carcinoma. In some embodiments, the cancer is esophagogastric adenocarcinoma. In some embodiments, the cancer is gastroesophageal junction adenocarcinoma. In some embodiments, the cancer is glioblastoma. In some embodiments, the cancer is head and neck squamous cell carcinoma. In some embodiments, the cancer is hepatocellular carcinoma (HCC). In some embodiments, the cancer is invasive breast carcinoma. In some embodiments, the cancer is leukemia. In some embodiments, the cancer is mature B-cell neoplasms. In some embodiments, the cancer is melanoma. In some embodiments, the cancer is mesothelioma. In some embodiments, the cancer is miscellaneous neuroepithelial tumor. In some embodiments, the cancer is non-seminomatous germ cell tumor. In some embodiments, the cancer is non-small cell lung cancer. In some embodiments, the cancer is ocular melanoma. In some embodiments, the cancer is ovarian cancer. In some embodiments, the cancer is ovarian epithelial tumor. In some embodiments, the cancer is pancreatic adenocarcinoma. In some embodiments, the cancer is pancreatic ductal adenocarcinoma. In some embodiments, the cancer is pheochromocytoma. In some embodiments, the cancer is pleural mesothelioma. In some embodiments, the cancer is prostate adenocarcinoma. In some embodiments, the cancer is prostate cancer. In some embodiments, the cancer is renal clear cell carcinoma. In some embodiments, the cancer is renal non-clear cell carcinoma. In some embodiments, the cancer is sarcoma. In some embodiments, the cancer is seminoma. In some embodiments, the cancer is thymic cancer. In some embodiments, the cancer is thymic epithelial tumor. In some embodiments, the cancer is thyroid cancer. In some embodiments, the cancer is undifferentiated stomach adenocarcinoma. In some embodiments, the cancer is well-differentiated thyroid cancer.

In some embodiments, the cancer is adrenocortical cancer comprising a dysfunction in the WNT pathway. In some embodiments, the cancer is adrenocortical carcinoma comprising a dysfunction in the WNT pathway. In some embodiments, the cancer is bladder cancer comprising a dysfunction in the WNT pathway. In some embodiments, the cancer is bladder urothelial carcinoma comprising a dysfunction in the WNT pathway. In some embodiments, the cancer is breast cancer comprising a dysfunction in the WNT pathway. In some embodiments, the cancer is cervical adenocarcinoma comprising a dysfunction in the WNT pathway. In some embodiments, the cancer is cervical squamous cell carcinoma comprising a dysfunction in the WNT pathway. In some embodiments, the cancer is cholangiocarcinoma comprising a dysfunction in the WNT pathway. In some embodiments, the cancer is colorectal adenocarcinoma comprising a dysfunction in the WNT pathway. In some embodiments, the cancer is colorectal cancer comprising a dysfunction in the WNT pathway. In some embodiments, the cancer is diffuse glioma comprising a dysfunction in the WNT pathway. In some embodiments, the cancer is endometrial cancer comprising a dysfunction in the WNT pathway. In some embodiments, the cancer is endometrial carcinoma comprising a dysfunction in the WNT pathway. In some embodiments, the cancer is esophageal squamous cell carcinoma comprising a dysfunction in the WNT pathway. In some embodiments, the cancer is esophagogastric adenocarcinoma comprising a dysfunction in the WNT pathway. In some embodiments, the cancer is gastroesophageal junction adenocarcinoma comprising a dysfunction in the WNT pathway. In some embodiments, the cancer is glioblastoma comprising a dysfunction in the WNT pathway. In some embodiments, the cancer is head and neck squamous cell carcinoma comprising a dysfunction in the WNT pathway. In some embodiments, the cancer is hepatocellular carcinoma (HCC) comprising a dysfunction in the WNT pathway. In some embodiments, the cancer is invasive breast carcinoma comprising a dysfunction in the WNT pathway. In some embodiments, the cancer is leukemia comprising a dysfunction in the WNT pathway. In some embodiments, the cancer is mature B-cell neoplasms comprising a dysfunction in the WNT pathway. In some embodiments, the cancer is melanoma comprising a dysfunction in the WNT pathway. In some embodiments, the cancer is mesothelioma comprising a dysfunction in the WNT pathway. In some embodiments, the cancer is miscellaneous neuroepithelial tumor comprising a dysfunction in the WNT pathway. In some embodiments, the cancer is non-seminomatous germ cell tumor comprising a dysfunction in the WNT pathway. In some embodiments, the cancer is non-small cell lung cancer comprising a dysfunction in the WNT pathway. In some embodiments, the cancer is ocular melanoma comprising a dysfunction in the WNT pathway. In some embodiments, the cancer is ovarian cancer comprising a dysfunction in the WNT pathway. In some embodiments, the cancer is ovarian epithelial tumor comprising a dysfunction in the WNT pathway. In some embodiments, the cancer is pancreatic adenocarcinoma comprising a dysfunction in the WNT pathway. In some embodiments, the cancer is pancreatic ductal adenocarcinoma comprising a dysfunction in the WNT pathway. In some embodiments, the cancer is pheochromocytoma comprising a dysfunction in the WNT pathway. In some embodiments, the cancer is pleural mesothelioma comprising a dysfunction in the WNT pathway. In some embodiments, the cancer is prostate adenocarcinoma comprising a dysfunction in the WNT pathway. In some embodiments, the cancer is prostate cancer comprising a dysfunction in the WNT pathway. In some embodiments, the cancer is renal clear cell carcinoma comprising a dysfunction in the WNT pathway. In some embodiments, the cancer is renal non-clear cell carcinoma comprising a dysfunction in the WNT pathway. In some embodiments, the cancer is sarcoma comprising a dysfunction in the WNT pathway. In some embodiments, the cancer is seminoma comprising a dysfunction in the WNT pathway. In some embodiments, the cancer is thymic cancer comprising a dysfunction in the WNT pathway. In some embodiments, the cancer is thymic epithelial tumor comprising a dysfunction in the WNT pathway. In some embodiments, the cancer is thyroid cancer comprising a dysfunction in the WNT pathway. In some embodiments, the cancer is undifferentiated stomach adenocarcinoma comprising a dysfunction in the WNT pathway. In some embodiments, the cancer is well-differentiated thyroid cancer comprising a dysfunction in the WNT pathway.

In some embodiments, the cancer is adrenocortical cancer comprising an activated WNT pathway. In some embodiments, the cancer is adrenocortical carcinoma comprising an activated WNT pathway. In some embodiments, the cancer is bladder cancer comprising an activated WNT pathway. In some embodiments, the cancer is bladder urothelial carcinoma comprising an activated WNT pathway. In some embodiments, the cancer is breast cancer comprising an activated WNT pathway. In some embodiments, the cancer is cervical adenocarcinoma comprising an activated WNT pathway. In some embodiments, the cancer is cervical squamous cell carcinoma comprising an activated WNT pathway. In some embodiments, the cancer is cholangiocarcinoma comprising an activated WNT pathway. In some embodiments, the cancer is colorectal adenocarcinoma comprising an activated WNT pathway. In some embodiments, the cancer is colorectal cancer comprising an activated WNT pathway. In some embodiments, the cancer is diffuse glioma comprising an activated WNT pathway. In some embodiments, the cancer is endometrial cancer comprising an activated WNT pathway. In some embodiments, the cancer is endometrial carcinoma comprising an activated WNT pathway. In some embodiments, the cancer is esophageal squamous cell carcinoma comprising an activated WNT pathway. In some embodiments, the cancer is esophagogastric adenocarcinoma comprising an activated WNT pathway. In some embodiments, the cancer is gastroesophageal junction adenocarcinoma comprising an activated WNT pathway. In some embodiments, the cancer is glioblastoma comprising an activated WNT pathway. In some embodiments, the cancer is head and neck squamous cell carcinoma comprising an activated WNT pathway. In some embodiments, the cancer is hepatocellular carcinoma (HCC) comprising an activated WNT pathway. In some embodiments, the cancer is invasive breast carcinoma comprising an activated WNT pathway. In some embodiments, the cancer is leukemia comprising an activated WNT pathway. In some embodiments, the cancer is mature B-cell neoplasms comprising an activated WNT pathway. In some embodiments, the cancer is melanoma comprising an activated WNT pathway. In some embodiments, the cancer is mesothelioma comprising an activated WNT pathway. In some embodiments, the cancer is miscellaneous neuroepithelial tumor comprising an activated WNT pathway. In some embodiments, the cancer is non-seminomatous germ cell tumor comprising an activated WNT pathway. In some embodiments, the cancer is non-small cell lung cancer comprising an activated WNT pathway. In some embodiments, the cancer is ocular melanoma comprising an activated WNT pathway. In some embodiments, the cancer is ovarian cancer comprising an activated WNT pathway. In some embodiments, the cancer is ovarian epithelial tumor comprising an activated WNT pathway. In some embodiments, the cancer is pancreatic adenocarcinoma comprising an activated WNT pathway. In some embodiments, the cancer is pancreatic ductal adenocarcinoma comprising an activated WNT pathway. In some embodiments, the cancer is pheochromocytoma comprising an activated WNT pathway. In some embodiments, the cancer is pleural mesothelioma comprising an activated WNT pathway. In some embodiments, the cancer is prostate adenocarcinoma comprising an activated WNT pathway. In some embodiments, the cancer is prostate cancer comprising an activated WNT pathway. In some embodiments, the cancer is renal clear cell carcinoma comprising an activated WNT pathway. In some embodiments, the cancer is renal non-clear cell carcinoma comprising an activated WNT pathway. In some embodiments, the cancer is sarcoma comprising an activated WNT pathway. In some embodiments, the cancer is seminoma comprising an activated WNT pathway. In some embodiments, the cancer is thymic cancer comprising an activated WNT pathway. In some embodiments, the cancer is thymic epithelial tumor comprising an activated WNT pathway. In some embodiments, the cancer is thyroid cancer comprising an activated WNT pathway. In some embodiments, the cancer is undifferentiated stomach adenocarcinoma comprising an activated WNT pathway. In some embodiments, the cancer is well-differentiated thyroid cancer comprising an activated WNT pathway.

In some embodiments, the cancer is adrenocortical cancer that has a high mutational burden. In some embodiments, the cancer is adrenocortical carcinoma that has a high mutational burden. In some embodiments, the cancer is bladder cancer that has a high mutational burden. In some embodiments, the cancer is bladder urothelial carcinoma that has a high mutational burden. In some embodiments, the cancer is breast cancer that has a high mutational burden. In some embodiments, the cancer is cervical adenocarcinoma that has a high mutational burden. In some embodiments, the cancer is cervical squamous cell carcinoma that has a high mutational burden. In some embodiments, the cancer is cholangiocarcinoma that has a high mutational burden. In some embodiments, the cancer is colorectal adenocarcinoma that has a high mutational burden. In some embodiments, the cancer is colorectal cancer that has a high mutational burden. In some embodiments, the cancer is diffuse glioma that has a high mutational burden. In some embodiments, the cancer is endometrial cancer that has a high mutational burden. In some embodiments, the cancer is endometrial carcinoma that has a high mutational burden. In some embodiments, the cancer is esophageal squamous cell carcinoma that has a high mutational burden. In some embodiments, the cancer is esophagogastric adenocarcinoma that has a high mutational burden. In some embodiments, the cancer is gastroesophageal junction adenocarcinoma that has a high mutational burden. In some embodiments, the cancer is glioblastoma that has a high mutational burden. In some embodiments, the cancer is head and neck squamous cell carcinoma that has a high mutational burden. In some embodiments, the cancer is hepatocellular carcinoma (HCC) that has a high mutational burden. In some embodiments, the cancer is invasive breast carcinoma that has a high mutational burden. In some embodiments, the cancer is leukemia that has a high mutational burden. In some embodiments, the cancer is mature B-cell neoplasms that has a high mutational burden. In some embodiments, the cancer is melanoma that has a high mutational burden. In some embodiments, the cancer is mesothelioma that has a high mutational burden. In some embodiments, the cancer is miscellaneous neuroepithelial tumor that has a high mutational burden. In some embodiments, the cancer is non-seminomatous germ cell tumor that has a high mutational burden. In some embodiments, the cancer is non-small cell lung cancer that has a high mutational burden. In some embodiments, the cancer is ocular melanoma that has a high mutational burden. In some embodiments, the cancer is ovarian cancer that has a high mutational burden. In some embodiments, the cancer is ovarian epithelial tumor that has a high mutational burden. In some embodiments, the cancer is pancreatic adenocarcinoma that has a high mutational burden. In some embodiments, the cancer is pancreatic ductal adenocarcinoma that has a high mutational burden. In some embodiments, the cancer is pheochromocytoma that has a high mutational burden. In some embodiments, the cancer is pleural mesothelioma that has a high mutational burden. In some embodiments, the cancer is prostate adenocarcinoma that has a high mutational burden. In some embodiments, the cancer is prostate cancer that has a high mutational burden. In some embodiments, the cancer is renal clear cell carcinoma that has a high mutational burden. In some embodiments, the cancer is renal non-clear cell carcinoma that has a high mutational burden. In some embodiments, the cancer is sarcoma that has a high mutational burden. In some embodiments, the cancer is seminoma that has a high mutational burden. In some embodiments, the cancer is thymic cancer that has a high mutational burden. In some embodiments, the cancer is thymic epithelial tumor that has a high mutational burden. In some embodiments, the cancer is thyroid cancer that has a high mutational burden. In some embodiments, the cancer is undifferentiated stomach adenocarcinoma that has a high mutational burden. In some embodiments, the cancer is well-differentiated thyroid cancer that has a high mutational burden.

In some embodiments, the cancer is adrenocortical cancer that has not responded to immunotherapy. In some embodiments, the cancer is adrenocortical carcinoma that has not responded to immunotherapy. In some embodiments, the cancer is bladder cancer that has not responded to immunotherapy. In some embodiments, the cancer is bladder urothelial carcinoma that has not responded to immunotherapy. In some embodiments, the cancer is breast cancer that has not responded to immunotherapy. In some embodiments, the cancer is cervical adenocarcinoma that has not responded to immunotherapy. In some embodiments, the cancer is cervical squamous cell carcinoma that has not responded to immunotherapy. In some embodiments, the cancer is cholangiocarcinoma that has not responded to immunotherapy. In some embodiments, the cancer is colorectal adenocarcinoma that has not responded to immunotherapy. In some embodiments, the cancer is colorectal cancer that has not responded to immunotherapy. In some embodiments, the cancer is diffuse glioma that has not responded to immunotherapy. In some embodiments, the cancer is endometrial cancer that has not responded to immunotherapy. In some embodiments, the cancer is endometrial carcinoma that has not responded to immunotherapy. In some embodiments, the cancer is esophageal squamous cell carcinoma that has not responded to immunotherapy. In some embodiments, the cancer is esophagogastric adenocarcinoma that has not responded to immunotherapy. In some embodiments, the cancer is gastroesophageal junction adenocarcinoma that has not responded to immunotherapy. In some embodiments, the cancer is glioblastoma that has not responded to immunotherapy. In some embodiments, the cancer is head and neck squamous cell carcinoma that has not responded to immunotherapy. In some embodiments, the cancer is hepatocellular carcinoma (HCC) that has not responded to immunotherapy. In some embodiments, the cancer is invasive breast carcinoma that has not responded to immunotherapy. In some embodiments, the cancer is leukemia that has not responded to immunotherapy. In some embodiments, the cancer is mature B-cell neoplasms that has not responded to immunotherapy. In some embodiments, the cancer is melanoma that has not responded to immunotherapy. In some embodiments, the cancer is mesothelioma that has not responded to immunotherapy. In some embodiments, the cancer is miscellaneous neuroepithelial tumor that has not responded to immunotherapy. In some embodiments, the cancer is non-seminomatous germ cell tumor that has not responded to immunotherapy. In some embodiments, the cancer is non-small cell lung cancer that has not responded to immunotherapy. In some embodiments, the cancer is ocular melanoma that has not responded to immunotherapy. In some embodiments, the cancer is ovarian cancer that has not responded to immunotherapy. In some embodiments, the cancer is ovarian epithelial tumor that has not responded to immunotherapy. In some embodiments, the cancer is pancreatic adenocarcinoma that has not responded to immunotherapy. In some embodiments, the cancer is pancreatic ductal adenocarcinoma that has not responded to immunotherapy. In some embodiments, the cancer is pheochromocytoma that has not responded to immunotherapy. In some embodiments, the cancer is pleural mesothelioma that has not responded to immunotherapy. In some embodiments, the cancer is prostate adenocarcinoma that has not responded to immunotherapy. In some embodiments, the cancer is prostate cancer that has not responded to immunotherapy. In some embodiments, the cancer is renal clear cell carcinoma that has not responded to immunotherapy. In some embodiments, the cancer is renal non-clear cell carcinoma that has not responded to immunotherapy. In some embodiments, the cancer is sarcoma that has not responded to immunotherapy. In some embodiments, the cancer is seminoma that has not responded to immunotherapy. In some embodiments, the cancer is thymic cancer that has not responded to immunotherapy. In some embodiments, the cancer is thymic epithelial tumor that has not responded to immunotherapy. In some embodiments, the cancer is thyroid cancer that has not responded to immunotherapy. In some embodiments, the cancer is undifferentiated stomach adenocarcinoma that has not responded to immunotherapy. In some embodiments, the cancer is well-differentiated thyroid cancer that has not responded to immunotherapy.

In some embodiments, the cancer is adrenocortical cancer that has not responded to or resistant to checkpoint inhibitor therapy. In some embodiments, the cancer is adrenocortical carcinoma that has not responded to checkpoint inhibitor therapy. In some embodiments, the cancer is bladder cancer that has not responded to checkpoint inhibitor therapy. In some embodiments, the cancer is bladder urothelial carcinoma that has not responded to checkpoint inhibitor therapy. In some embodiments, the cancer is breast cancer that has not responded to checkpoint inhibitor therapy. In some embodiments, the cancer is cervical adenocarcinoma that has not responded to checkpoint inhibitor therapy. In some embodiments, the cancer is cervical squamous cell carcinoma that has not responded to checkpoint inhibitor therapy. In some embodiments, the cancer is cholangiocarcinoma that has not responded to checkpoint inhibitor therapy. In some embodiments, the cancer is colorectal adenocarcinoma that has not responded to checkpoint inhibitor therapy. In some embodiments, the cancer is colorectal cancer that has not responded to checkpoint inhibitor therapy. In some embodiments, the cancer is diffuse glioma that has not responded to checkpoint inhibitor therapy. In some embodiments, the cancer is endometrial cancer that has not responded to checkpoint inhibitor therapy. In some embodiments, the cancer is endometrial carcinoma that has not responded to checkpoint inhibitor therapy. In some embodiments, the cancer is esophageal squamous cell carcinoma that has not responded to checkpoint inhibitor therapy. In some embodiments, the cancer is esophagogastric adenocarcinoma that has not responded to checkpoint inhibitor therapy. In some embodiments, the cancer is gastroesophageal junction adenocarcinoma that has not responded to checkpoint inhibitor therapy. In some embodiments, the cancer is glioblastoma that has not responded to checkpoint inhibitor therapy. In some embodiments, the cancer is head and neck squamous cell carcinoma that has not responded to checkpoint inhibitor therapy. In some embodiments, the cancer is hepatocellular carcinoma (HCC) that has not responded to checkpoint inhibitor therapy. In some embodiments, the cancer is invasive breast carcinoma that has not responded to checkpoint inhibitor therapy. In some embodiments, the cancer is leukemia that has not responded to checkpoint inhibitor therapy. In some embodiments, the cancer is mature B-cell neoplasms that has not responded to checkpoint inhibitor therapy. In some embodiments, the cancer is melanoma that has not responded to checkpoint inhibitor therapy. In some embodiments, the cancer is mesothelioma that has not responded to checkpoint inhibitor therapy. In some embodiments, the cancer is miscellaneous neuroepithelial tumor that has not responded to checkpoint inhibitor therapy. In some embodiments, the cancer is non-seminomatous germ cell tumor that has not responded to checkpoint inhibitor therapy. In some embodiments, the cancer is non-small cell lung cancer that has not responded to checkpoint inhibitor therapy. In some embodiments, the cancer is ocular melanoma that has not responded to checkpoint inhibitor therapy. In some embodiments, the cancer is ovarian cancer that has not responded to checkpoint inhibitor therapy. In some embodiments, the cancer is ovarian epithelial tumor that has not responded to checkpoint inhibitor therapy. In some embodiments, the cancer is pancreatic adenocarcinoma that has not responded to checkpoint inhibitor therapy. In some embodiments, the cancer is pancreatic ductal adenocarcinoma that has not responded to checkpoint inhibitor therapy. In some embodiments, the cancer is pheochromocytoma that has not responded to checkpoint inhibitor therapy. In some embodiments, the cancer is pleural mesothelioma that has not responded to checkpoint inhibitor therapy. In some embodiments, the cancer is prostate adenocarcinoma that has not responded to checkpoint inhibitor therapy. In some embodiments, the cancer is prostate cancer that has not responded to checkpoint inhibitor therapy. In some embodiments, the cancer is renal clear cell carcinoma that has not responded to checkpoint inhibitor therapy. In some embodiments, the cancer is renal non-clear cell carcinoma that has not responded to checkpoint inhibitor therapy. In some embodiments, the cancer is sarcoma that has not responded to checkpoint inhibitor therapy. In some embodiments, the cancer is seminoma that has not responded to checkpoint inhibitor therapy. In some embodiments, the cancer is thymic cancer that has not responded to checkpoint inhibitor therapy. In some embodiments, the cancer is thymic epithelial tumor that has not responded to checkpoint inhibitor therapy. In some embodiments, the cancer is thyroid cancer that has not responded to checkpoint inhibitor therapy. In some embodiments, the cancer is undifferentiated stomach adenocarcinoma that has not responded to checkpoint inhibitor therapy. In some embodiments, the cancer is well-differentiated thyroid cancer that has not responded to checkpoint inhibitor therapy.

### Methods of Inducing or Enhancing Immune Response

The disclosure further provides a method of inducing a neoplasia-specific or tumor-specific immune response in a subject, vaccinating against a neoplasia/tumor, treating and/or alleviating a symptom of cancer in a subject by administering to the subject the nucleic acid sequences as described herein. In some embodiments, the present disclosure relates to a method of inducing an immune response in a subject, the method comprising administering to the subject in need thereof a pharmaceutically effective amount of any of the nucleic acid molecules of any one of the aspects or embodiments herein, or any one of the pharmaceutical compositions of any one of the aspects and embodiments herein. In some embodiments, the method comprises the steps of taking a sample from a subject, identifying one or more neoantigens expressed by cancer cells in a sample, synthesizing one or more cDNA libraries based upon expression of neoantigens in the sample, cloning the one or more nucleic acid sequences that encode one or more epitopes of the neoantigens into a nucleic acid molecule that comprises one or more components disclosed herein, and administering the nucleic acid molecule to the subject.

In some embodiments the present disclosure features a method of inducing a CD8+ T cell immune response in a subject, the method comprising administering to the subject in need thereof a pharmaceutically effective amount of any of the nucleic acid molecules of any one of the aspects or embodiments herein, or any one of the pharmaceutical compositions of any one of the aspects and embodiments herein.

The present disclosure features a method of enhancing an immune response in a subject, the method comprising administering to the subject in need thereof a pharmaceutically effective amount of any of the nucleic acid molecules of any one of the aspects or embodiments herein, or any one of the pharmaceutical compositions of any one of the aspects and embodiments herein.

In some aspects, the present disclosure features a method of enhancing a CD8+ T cell immune response in a subject, the method comprising administering to the subject in need thereof a pharmaceutically effective amount of any of the nucleic acid molecules of any one of the aspects or embodiments herein, or any one of the pharmaceutical compositions of any one of the aspects and embodiments herein.

In some embodiments, the subject has cancer. In another embodiment, the subject has previously been treated, and not responded to checkpoint inhibitor therapy.

In some embodiments, the nucleic acid molecule is administered to the subject by electroporation.

In some embodiments, enhancing the CD8+ T cell immune response comprises activating from about 0.01% to about 50% CD8+ T cells. In some embodiments, enhancing the CD8+ T cell immune response comprises activating from about 0.01% to about 50% that are IFN-γ positive. In some embodiments, the activation of T cells is accomplished after no more than 1, 2, 3, 4, 5, 6, 7, 8, 9, 10 or more hours of contact with antigen presenting cells expressing or plasmids comprising the nucleic acid sequences disclosed herein or expressed by a hyperproliferative cell in a subject. CD8+ T cells. In some embodiments, enhancing the CD8+ T cell immune response comprises expanding CD8+ T cells. In some embodiments, enhancing the CD8+ T cell immune response comprises activating from about 0.05% to about 50% CD8+ T cells. In some embodiments, enhancing the CD8+ T cell immune response comprises activating from about 0.10% to about 50% CD8+ T cells. In some embodiments, enhancing the CD8+ T cell immune response comprises activating from about 0.2% to about 50% CD8+ T cells. In some embodiments, enhancing the CD8+ T cell immune response comprises activating from about 0.3% to about 50% CD8+ T cells. In some embodiments, enhancing the CD8+ T cell immune response comprises activating from about 0.4% to about 50% CD8+ T cells. In some embodiments, enhancing the CD8+ T cell immune response comprises activating from about 0.5% to about 50% CD8+ T cells. In some embodiments, enhancing the CD8+ T cell immune response comprises activating from about 0.6% to about 50% CD8+ T cells. In some embodiments, enhancing the CD8+ T cell immune response comprises activating from about 0.7% to about 50% CD8+ T cells. In some embodiments, enhancing the CD8+ T cell immune response comprises activating from about 0.8% to about 50% CD8+ T cells. In some embodiments, enhancing the CD8+ T cell immune response comprises activating from about 0.9% to about 50% CD8+ T cells. In some embodiments, enhancing the CD8+ T cell immune response comprises activating from about 1.00% to about 50% CD8+ T cells. In some embodiments, enhancing the CD8+ T cell immune response comprises activating from about 2.0% to about 50% CD8+ T cells. In some embodiments, enhancing the CD8+ T cell immune response comprises activating from about 3.0% to about 50% CD8+ T cells. In some embodiments, enhancing the CD8+ T cell immune response comprises activating from about 5.0% to about 50% CD8+ T cells. In some embodiments, enhancing the CD8+ T cell immune response comprises activating from about 6.0% to about 50% CD8+ T cells. In some embodiments, enhancing the CD8+ T cell immune response comprises activating from about 7.0% to about 50% CD8+ T cells. In some embodiments, enhancing the CD8+ T cell immune response comprises activating from about 8.0% to about 50% CD8+ T cells. In some embodiments, enhancing the CD8+ T cell immune response comprises activating from about 9.0% to about 50% CD8+ T cells. In some embodiments, enhancing the CD8+ T cell immune response comprises activating from about 10% to about 50% CD8+ T cells. In some embodiments, enhancing the CD8+ T cell immune response comprises activating from about 15% to about 50% CD8+ T cells. In some embodiments, enhancing the CD8+ T cell immune response comprises activating from about 20% to about 50% CD8+ T cells.

T cell activation can be measured by various assays as described herein. For example, T cell activities that may be measured include the induction of proliferation of T cells, the induction of signaling in T cells, the induction of expression of activation markers in T cells, such as interferon-gamma (IFN-s), the induction of cytokine secretion by T cells, and the cytotoxic activity of T cells. For example, in certain embodiments, CD8+ T cell activation is measured by a proliferation assay. In some embodiments, the activation may be measured after stimulation of cells or cell sample by the encoded nucleic acid sequences.

The activation of CD8+ T-cells may be assessed or measured by determining secretion of cytokines, such as gamma interferon (IFN-γ), tumor necrosis factor alpha (TNFα), interleukin-12 (IL-12) or interleukin 2 (IL-2). In some embodiments, ELISA is used to determine cytokine secretion, for example secretion of gamma interferon (IFN-γ), tumor necrosis factor alpha (TNFα), interleukin-12 (IL-12) or interleukin 2 (IL-2). The ELISPOT (enzyme-linked immunospot) technique may be used to detect T cells that secrete a given cytokine (e.g., gamma interferon (IFN-γ)) in response to stimulation with any of the nucleic acid molecules of any one of the aspects or embodiments herein, or any one of the pharmaceutical compositions of any one of the aspects and embodiments herein. T cells are cultured with, e.g. any of the nucleic acid molecules of any one of the aspects or embodiments herein wells which have been coated with anti-IFN-γ antibodies. The secreted IFN-γ is captured by the coated antibody and then revealed with a second antibody coupled to a chromogenic substrate. Thus, locally secreted cytokine molecules form spots, with each spot corresponding to one IFN-γ-secreting cell. The number of spots allows one to determine the frequency of IFN-γ-secreting cells in the analyzed sample. The ELISPOT assay has also been described for the detection of tumor necrosis factor alpha, interleukin-4 (IL-4), IL-5, IL-6, IL-10, IL-12, granulocyte-macrophage colony-stimulating factor, and granzyme B-secreting lymphocytes (Klinman D, Nutman T. Current protocols in immunology. New York, N.Y: John Wiley & Sons, Inc.; 1994. pp. 6.19.1-6.19.8, incorporated by reference in its entirety herein).

Flow cytometric analyses of intracellular cytokines may be used to measure the cytokine content in culture supernatants but provides no information on the number of T cells that actually secrete the cytokine. When T cells are treated with inhibitors of secretion such as monensin or brefeldin A, they accumulate cytokines within their cytoplasm upon activation (e.g. with the nucleic acid molecules of the present disclosure). After fixation and permeabilization of the lymphocytes, intracellular cytokines can be quantified by cytometry. This technique allows the determination of the cytokines produced, the type of cells that produce these cytokines, and the quantity of cytokine produced per cell.

The activation of CD8+ T-cells by any of the nucleic acid molecules of any one of the aspects or embodiments herein, or any one of the pharmaceutical compositions of any one of the aspects and embodiments herein may be assessed by assaying the cytotoxic activity of the CD8+ T-cells.

The cytotoxic activity of T cells may be assessed by any suitable technique known to those of skill in the art. For example, a sample comprising T cells that have been exposed to the nucleic acid molecules according to the disclosure can be assayed for cytotoxic activity after an appropriate period of time, in a standard cytotoxic assay. Such assays may include, but are not limited to, the chromium release CTL assay and the Alamar Blue™ fluorescence assay known in the art.

The ability of the any of the nucleic acid molecules of any one of the aspects or embodiments herein, or any one of the pharmaceutical compositions of any one of the aspects and embodiments herein to expand T cells can be evaluated by using CFSE staining. To compare the initial rate of cell expansion, the cells are subject to CFSE staining to determine how well any of the nucleic acid molecules of any one of the aspects or embodiments herein, or any one of the pharmaceutical compositions of any one of the aspects and embodiments herein induced the proliferation of T cells. CFSE staining provides a much more quantitative endpoint and allows simultaneous phenotyping of the expanded cells. Every day after stimulation, an aliquot of cells is removed from each culture and analyzed by flow cytometry. CFSE staining makes cells highly fluorescent. Upon cell division, the fluorescence is halved and thus the more times a cell divides the less fluorescent it becomes. The ability of any of the nucleic acid molecules of any one of the aspects or embodiments herein, or any one of the pharmaceutical compositions of any one of the aspects and embodiments herein to induce T cell proliferation is quantitated by measuring the number of cells that divided once, twice, three times and so on. The nucleic acid molecules that induce the greatest number of cell divisions at a particular time point is deemed as the most potent expander.

To determine how well any of the nucleic acid molecules of any one of the aspects or embodiments herein, or any one of the pharmaceutical compositions of any one of the aspects and embodiments herein promote long-term growth of T cells, cell growth curves can be generated. These experiments are set up as the foregoing CFSE experiments, but no CFSE is used. Every 2-3 days of culture, T cells are removed from the respective cultures and counted using a Coulter counter which measures how many cells are present and the mean volume of the cells. The mean cell volume is the best predicator of when to restimulate the cells. In general, when T cells are properly stimulated they triple their cell volume. When this volume is reduced to more than about half of the initial blast, it may be necessary to restimulate the T cells to maintain a log linear expansion (Levine et al., 1996, Science 272:1939-1943; Levine et al., 1997, J. Immunol. 159:5921-5930). The time it takes the nucleic acid molecules of any one of the aspects or embodiments herein, or any one of the pharmaceutical compositions of any one of the aspects and embodiments herein to induce 20 population doublings is calculated. The relative differences of each nucleic acid molecule to induce this level of T cell expansion is an important criteria on which the nucleic acid molecules of any one of the aspects or embodiments herein, or any one of the pharmaceutical compositions of any one of the aspects and embodiments herein are assessed.

In certain embodiments of the present disclosure, stimulation, activation, and expansion of T cells using the nucleic acid molecules of any one of the aspects or embodiments herein, or any one of the pharmaceutical compositions of any one of the aspects and embodiments herein enhances expression of certain key molecules in T cells that protect again apoptosis or otherwise prolong survival in vivo or in vitro. Apoptosis usually results from induction of a specific signal in the T cell. Thus, the nucleic acid molecules of any one of the aspects or embodiments herein, or any one of the pharmaceutical compositions of any one of the aspects and embodiments herein may provide for protecting a T cell from cell death resulting from stimulation of the T cell. Therefore, also included in the present disclosure is the enhanced T cell growth by protection from premature death or from absence or depletion of recognized T cell growth markers, such as Bcl-xL, growth factors, cytokines, or lymphokines normally necessary for T cell survival, as well as from Fas or Tumor Necrosis Factor Receptor (TNFR) cross-linking or by exposure to certain hormones or stress.

In other embodiments, the disclosure features a method of enhancing an immune response against a plurality of heterogeneous hyperproliferative cells or cancer cells in a subject, the method comprising administering to the subject in need thereof a pharmaceutically effective amount of any of the nucleic acid molecules described herein (e.g. a nucleic acid molecule comprising a nucleic acid sequence comprising Formula I: [(AEDn)-(linker)]n-[AEDn+1]), or any of the pharmaceutical compositions described herein.

In some embodiments, the subject has cancer. In another embodiment, the subject has previously been treated, and not responded to checkpoint inhibitor therapy.

In some embodiments, the nucleic acid molecule is administered to the subject by electroporation.

In some embodiments, the immune response is of a sufficient magnitude or efficacy to inhibit or retard tumor growth, induce tumor cell death, induce tumor regression, prevent or delay tumor recurrence, prevent tumor growth, prevent tumor spread and/or induce tumor elimination.

In some embodiments, the method of enhancing an immune response against a plurality of heterogeneous hyperproliferative cells or cancer cells in a subject further comprises administration of one or more therapeutic agents in addition to the pharmaceutical composition disclosed herein.

In some embodiments, the additional therapeutic agent is a biologic therapeutic or a small molecule.

In another embodiment, the therapeutic agent is (i) a checkpoint inhibitor or functional fragment thereof; or (ii) a nucleic acid molecule encoding a checkpoint inhibitor or a functional fragment thereof. In a further embodiment, the checkpoint inhibitor associates with or inhibits a checkpoint protein selected from the group consisting of CTLA-4, PDL1, PDL2, PDl, B7-H3, B7-H4, BTLA, HVEM, TIM3, GAL9, LAG3, VISTA, KIR, 2B4, CD160, CGEN-15049, CHK 1, CHK2, A2aR, and B-7 family ligands or a combination thereof. In an exemplary embodiment, the checkpoint inhibitor is an inhibitor of the programmed death-1 (PD-1) pathway. In another exemplary embodiment, the checkpoint inhibitor is an anti-cytotoxic T-lymphocyte-associated antigen 4 (CTLA4) antibody or functional fragment thereof.

In another embodiment, the therapeutic agent is an adjuvant. The ability of an adjuvant to increase the immune response to an antigen is typically manifested by a significant increase in immune-mediated reaction, or reduction in disease symptoms. For example, an increase in humoral immunity is typically manifested by a significant increase in the titer of antibodies raised to the antigen, and an increase in T-cell activity is typically manifested in increased cell proliferation, or cellular cytotoxicity, or cytokine secretion. An adjuvant may also alter an immune response, for example, by changing a primarily humoral or Th2 response into a primarily cellular, or Th1 response. In some embodiments, the adjuvant can be other genes that are expressed in alternative plasmid or are delivered as proteins in combination with the plasmid above in the vaccine.

In some embodiments, the adjuvant can be selected from the group consisting of: α-interferon (IFN-α), β-interferon (IFN-β), y-interferon, platelet derived growth factor (PDGF), TNFα, TNFβ, GM-CSF, epidermal growth factor (EGF), cutaneous T cell-attracting chemokine (CTACK), epithelial thymus-expressed chemokine (TECK), mucosae-associated epithelial chemokine (MEC), IL-12, IL-15, MHC, CD80, CD86 including IL-15 having the signal sequence deleted and optionally including the signal peptide from IgE. The adjuvant can be IL-12, IL-15, IL-28, CTACK, TECK, platelet derived growth factor (PDGF), TNFα, TNFβ, GM-CSF, epidermal growth factor (EGF), IL-1, IL-2, IL-4, IL-5, IL-6, IL-10, IL-12, IL-18, or a combination thereof.

Other genes which can be useful adjuvants include those encoding: MCP-1, MIP-1a, MIP-1p, IL-8, RANTES, L-selectin, P-selectin, E-selectin, CD34, GlyCAM-1, MadCAM-1, LFA-1, VLA-1, Mac-1, p150.95, PECAM, ICAM-1, ICAM-2, ICAM-3, CD2, LFA-3, M-CSF, G-CSF, IL-4, mutant forms of IL-18, CD40, CD40L, vascular growth factor, fibroblast growth factor, IL-7, nerve growth factor, vascular endothelial growth factor, Fas, TNF receptor, Flt, Apo-1, p55, WSL-1, DR3, TRAMP, Apo-3, AIR, LARD, NGRF, DR4, DR5, KILLER, TRAIL-R2, TRICK2, DR6, Caspase ICE, Fos, c-jun, Sp-1, Ap-1, Ap-2, p38, p65Rel, MyD88, IRAK, TRAF6, IkB, Inactive NIK, SAP K, SAP-1, JNK, interferon response genes, NFkB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP1, TAP2 and functional fragments thereof.

Human IL-12 alpha subunit is set forth in GenBank Accession Nos. NP_000873.2, NM_000882.3, incorporated by reference in their entireties herein. An exemplary human IL-12 alpha subunit amino acid sequence is shown below:

(SEQ ID NO: 56)
MCPARSLLLVATLVLLDHLSLARNLPVATPDPGMFPCLHHSQNLLRAVS
NMLQKARQTLEFYPCTSEEIDHEDITKDKTSTVEACLPLELTKNESCLN
SRETSFITNGSCLASRKTSFMMALCLSSIYEDLKMYQVEFKTMNAKLLM
DPKRQIFLDQNMLAVIDELMQALNFNSETVPQKSSLEEPDFYKTKIKLC
ILLHAFRIRAVTIDRVMSYLNAS.

An exemplary DNA sequence encoding SEQ ID NO: 56 is:

(SEQ ID NO. 68)
atgtgtccagcgcgcagcctcctccttgtggctaccctggtcctc
ctggaccacctcagtttggccagaaacctccccgtggccactcca
gacccaggaatgttcccatgccttcaccactcccaaaacctgctg
agggccgtcagcaacatgctccagaaggccagacaaactctagaa
ttttacccttgcacttctgaagagattgatcatgaagatatcaca
aaagataaaaccagcacagtggaggcctgtttaccattggaatta
accaagaatgagagttgcctaaattccagagagacctctttcata
actaatgggagttgcctggcctccagaaagacctcttttatgatg
gccctgtgccttagtagtatttatgaagacttgaagatgtaccag
gtggagttcaagaccatgaatgcaaagcttctgatggatcctaag
aggcagatctttctagatcaaaacatgctggcagttattgatgag
ctgatgcaggccctgaatttcaacagtgagactgtgccacaaaaa
tcctcccttgaagaaccggatttttataaaactaaaatcaagctc
tgcatacttcttcatgctttcagaattcgggcagtgactattgat
agagtgatgagctatctgaatgcttcctaa

Human IL-12 beta subunit is set forth in GenBank Accession No. NP_002178.2, incorporated by reference in its entirety herein. An exemplary human IL-12 beta subunit amino acid sequence is shown below:

(SEQ ID NO: 57)
MCHQQLVISWFSLVFLASPLVAIWELKKDVYVVELDWYPDAPGEM
VVLTCDTPEEDGITWTLDQSSEVLGSGKTLTIQVKEFGDAGQYTC
HKGGEVLSHSLLLLHKKEDGIWSTDILKDQKEPKNKTFLRCEAKN
YSGRFTCWWLTTISTDLTFSVKSSRGSSDPQGVTCGAATLSAERV
RGDNKEYEYSVECQEDSACPAAEESLPIEVMVDAVHKLKYENYTS
SFFIRDIIKPDPPKNLQLKPLKNSRQVEVSWEYPDTWSTPHSYFS
LTFCVQVQGKSKREKKDRVFTDKTSATVICRKNASISVRAQDRYY
SSSWSEWASVPCS

An exemplary DNA sequence encoding SEQ ID NO: 57 is:

(SEQ ID NO.: 69)
atgtgtcaccagcagttggtcatctcttggttttccctggttttt
ctggcatctcccctcgtggccatatgggaactgaagaaagatgtt
tatgtcgtagaattggattggtatccggatgcccctggagaaatg
gtggtcctcacctgtgacacccctgaagaagatggtatcacctgg
accttggaccagagcagtgaggtcttaggctctggcaaaaccctg
accatccaagtcaaagagtttggagatgctggccagtacacctgt
cacaaaggaggcgaggttctaagccattcgctcctgctgcttcac
aaaaaggaagatggaatttggtccactgatattttaaaggaccag
aaagaacccaaaaataagacctttctaagatgcgaggccaagaat
tattctggacgtttcacctgctggtggctgacgacaatcagtact
gatttgacattcagtgtcaaaagcagcagaggctcttctgacccc
caaggggtgacgtgcggagctgctacactctctgcagagagagtc
agaggggacaacaaggagtatgagtactcagtggagtgccaggag
gacagtgcctgcccagctgctgaggagagtctgcccattgaggtc
atggtggatgccgttcacaagctcaagtatgaaaactacaccagc
agcttcttcatcagggacatcatcaaacctgacccacccaagaac
ttgcagctgaagccattaaagaattctcggcaggtggaggtcagc
tgggagtaccctgacacctggagtactccacattcctacttctcc
ctgacattctgcgttcaggtccagggcaagagcaagagagaaaag
aaagatagagtcttcacggacaagacctcagccacggtcatctgc
cgcaaaaatgccagcattagcgtgcgggcccaggaccgctactat
agctcatcttggagcgaatgggcatctgtgccctgcagttag

Human IL-15 is set forth in GenBank Accession Nos. NP_000576.1, NP_751915.1, AAI00962.1 incorporated by reference in their entireties herein. An exemplary human IL-15 amino acid sequence is shown below:

(SEQ ID NO: 58)
MRISKPHLRSISIQCYLCLLLNSHFLTEAGIHVFILGCFSAGLPK
TEANWVNVISDLKKIEDLIQSMHIDATLYTESDVHPSCKVTAMKC
FLLELQVISLESGDASIHDTVENLIILANNSLSSNGNVTESGCKE
CEELEEKNIKEFLQSFVHIVQMFINTS

Human IL-17 is set forth in GenBank Accession Nos. NP_002181.1, NM_002190.2, incorporated by reference in their entireties herein. An exemplary human IL-17 amino acid sequence is shown below:

(SEQ ID NO: 59)
MTPGKTSLVSLLLLLSLEAIVKAGITIPRNPGCPNSEDKNFPRTV
MVNLNIHNRNTNTNPKRSSDYYNRSTSPWNLHRNEDPERYPSVIW
EAKCRHLGCINADGNVDYHMNSVPIQQEILVLRREPPHCPNSFRL
EKILVSVGCTCVTPIVHHVA

Human IL-8 is set forth in GenBank Accession Nos. NP_000575.1, NM_000584.3, incorporated by reference in their entireties herein. An exemplary human IL-8 amino acid sequence is shown below:

(SEQ ID NO: 60)
MTSKLAVALLAAFLISAALCEGAVLPRSAKELRCQCIKTYSKPFH
PKFIKELRVIESGPHCANTEIIVKLSDGRELCLDPKENWVQRVVE
KFLKRAENS

Human C-C motif chemokine 5 (processed form RANTES (3-68)) is set forth in GenBank Accession Nos. NP_002976.2, NM_002985.2, incorporated by reference in their entireties herein. An exemplary human C-C motif chemokine 5 amino acid sequence is shown below:

(SEQ ID NO: 61)
MKVSAAALAVILIATALCAPASASPYSSDTTPCCFAYIARPLPRA
HIKEYFYTSGKCSNPAVVFVTRKNRQVCANPEKKWVREYINSLEM
S

Human Macrophage inflammatory protein 1-alpha (MIP-1a) is set forth in GenBank Accession Nos. NP_002974.1, NM_002983.2, incorporated by reference in their entireties herein. An exemplary human C-C motif chemokine 5 amino acid sequence is shown below:

(SEQ ID NO: 62)
MQVSTAALAVLLCTMALCNQFSASLAADTPTACCFSYTSRQIPQN
FIADYFETSSQCSKPGVIFLTKRSRQVCADPSEEWVQKYVSDLEL
SA

Other exemplary adjuvants include, but are not limited to, poly-ICLC (see Pharmacol Ther. 2015 February; 146:120-31, incorporated by reference in its entirety herein), 1018 ISS (see Vaccine. 2003 Jun. 2; 21 (19-20): 2461-7, incorporated by reference in its entirety herein), aluminum salts, Amplivax AS15, Bacillus Colmette-Guérin (BCG) (see Clin Immunol. 2000 January; 94 (1): 64-72, incorporated by reference in its entirety herein), CP-870,893, CpG7909 (GenBank Accession No. CS576603.1), CyaA (GenBank Accession No. KP670536.1), GM-CSF (GenBank Accession No. M11220.1), IC30 (see Expert Rev Vaccines. 2007 October; 6 (5): 741-6, incorporated by reference in its entirety herein), IC31 (see Expert Rev Vaccines. 2007 October; 6 (5): 741-6, incorporated by reference in its entirety herein), Imiquimod (see Vaccine. 2006 Mar. 10; 24 (11): 1958-6, incorporated by reference in its entirety herein), ImuFact 1MP321, IS Patch, ISS, ISCOMATRIX, Juvlmmune, LipoVac, monophosphoryl lipid A, Montanide IMS 1312, Montanide ISA 206, Montanide ISA 50V, Montanide ISA-51, OK-432, OM-174, OM-197-MP-EC, ONTAK, PEPTEL, vector system, PLGA micropartieles, resiquimod, S L172, Virosomes and other Virus-like particles, YF-17D, VEGF trap, R848, beta-glucan, Pam3Cys, acrylic or methacrylic polymers, copolymers of maleic anhydride and Aquila's QS21 stimulon, and a functional fragment of any thereof; or (ii) a nucleic acid molecule encoding an adjuvant selected from the group consisting of: (i) poly-ICLC, 1018 ISS, aluminum salts, Amplivax AS15, BCG, CP-870,893, CpG7909, CyaA, GM-CSF, IC30, IC31, Imiquimod, ImuFact 1MP321, IS Patch, ISS, ISCOMATRIX, Juvlmmune, LipoVac, monophosphoryl lipid A, Montanide IMS 1312, Montanide ISA 206, Montanide ISA 50V, Montanide ISA-51, OK-432, OM-174, OM-197-MP-EC, ONTAK, PEPTEL, vector system, PLGA microparticles, resiquimod, S L172, Virosomes and other Virus-like particles, YF-17D, VEGF trap, R848, beta-glucan, Pam3Cys, acrylic or methacrylic polymers, copolymers of maleic anhydride and Aquila's QS21 stimulon, or functional fragment thereof.

In another embodiment, the therapeutic agent is an immunostimulatory agent or functional fragment thereof. For example, in some embodiments, the imunostimulatory agent is an interleukin or functional fragment thereof.

In another embodiment, the therapeutic agent is a chemotherapeutic agent. Examples of chemotherapeutic agents include, but are not limited to, aldesleukin, altretamine, amifostine, asparaginase, bleomycin, capecitabine, carboplatin, carmustine, cladribine, cisapride, cisplatin, cyclophosphamide, cytarabine, dacarbazine (DTIC), dactinomycin, docetaxel, doxorubicin, dronabinol, epoetin a, etoposide, filgrastim, fludarabine, fluorouracil, gemcitabine, granisetron, hydroxyurea, idarubicin, ifosfamide, interferon alpha, irinotecan, lansoprazole, levamisole, leucovorin, megestrol, mesna, methotrexate, metoclopramide, mitomycin, mitotane, mitoxantrone, omeprazole, ondansetron, paclitaxel (Taxol®), pilocarpine, prochloroperazine, rituximab, tamoxifen, taxol, topotecan hydrochloride, trastuzumab, vinblastine, vincristine and vinorelbine tartrate. For prostate cancer treatment, a chemotherapeutic agent with which anti-CTLA-4 can be combined is paclitaxel (Taxol®).

In some embodiments, the adjuvant can include a nucleic acid plasmid that encodes any cytokine or functional fragment thereof that is administered sequentially with a pharmaceutical composition comprising a plasmid encoding a plurality of neoantigens, optionally with one or a plurality of tumor associated antigens not derived from a subject. In some embodiments, the cytokine is IL-12 or a subunit of IL-12. In some embodiments, adjuvant is a nucleic acid sequence that encodes an amino acid sequence that comprises at least about 70%, 75%, 80%, 85%, 90%, 95%, 96%, 97%, 98%, 99% or 100% sequence identity to SEQ NO: 56 or a functional fragment thereof. In some embodiments, adjuvant is a nucleic acid sequence that encodes an amino acid sequence that comprises at least about 70%, 75%, 80%, 85%, 90%, 95%, 96%, 97%, 98%, 99% or 100% sequence identity to SEQ NO: 57 or a functional fragment thereof. In some embodiments, adjuvant is a first nucleic acid sequence that encodes an amino acid sequence that comprises at least about 70%, 75%, 80%, 85%, 90%, 95%, 96%, 97%, 98%, 99% or 100% sequence identity to SEQ NO: 56 and a second amino acid sequence that comprises at least about 70%, 75%, 80%, 85%, 90%, 95%, 96%, 97%, 98%, 99% or 100% sequence identity to SEQ NO: 57 or a functional fragment thereof. In some embodiments, if the nucleic acid sequence encoding a cytokine or functional fragment thereof comprise two subunits, the disclosure relates to nucleic acid molecule comprises a first nucleic acid sequence encoding the first subunit and a second nucleic acid encoding the second subunit, each of the first or second nucleic acid sequences operably linked to at least a first promoter, such as a CMV promoter. In some embodiments, if the nucleic acid sequence encoding a cytokine or functional fragment thereof comprise two subunits, the disclosure relates to nucleic acid molecule comprises a first nucleic acid sequence encoding the first subunit and a second nucleic acid encoding the second subunit, the first nucleic acid sequence is operably linked to at least a first promoter and the second nucleic acid sequence is operably linked to at least a second promoter.

In some embodiments, the IL-12 sequences and nucleic acids sequences encoding the same can be found in U.S. Pat. Nos. 9,981,036 and 9,272,024, each of which is incorporated by reference in its entirety.

Methods of Preventing Resistance to Immunotherapy and/or Checkpoint Inhibitor Therapy

The present disclosure relates to a method of reducing resistance or tolerance to immunotherapy in a subject, the method comprising administering to a subject in need thereof a pharmaceutical composition comprising a therapeutically effective amount of a composition comprising a nucleic acid sequence encoding at least about twenty tumor-specific epitopes disclosed herein, wherein the nucleic acid sequence is free of a nucleic acid sequence that encodes a WNT pathway tumor-specific epitope. In some embodiments, the method comprising administering to the subject an expressible nucleic acid sequence encoding at least about 20, 25, 30, 35, 40, 45, 50, 55 or more tumor-specific antigens, wherein the expressible nucleic acid sequence may or may not express at least one tumor-specific antigen disclosed herein. In some embodiments, the tumor-specific antigens are chosen from one or a plurality of amino acid sequences identified in the Examples or functional fragments thereof that comprise about 70%, 75%, 80%, 85%, 86%, 87%, 88%, 89%, 90%, 91%, 92%, 93%, 94%, 95%, 96%, 97%, 98%, 99% sequence identity to SEQ ID Nos in the Examples.

The present disclosure relates to a method of reducing resistance to immunotherapy in a subject, the method comprising administering to a subject in need thereof a pharmaceutical composition comprising a therapeutically effective amount of a composition comprising a nucleic acid sequence encoding an antigen associated with a dysfunctional WNT pathway.

The present disclosure features a method of abrogating resistance to checkpoint inhibitor therapy in a subject, the method comprising administering to a subject in need thereof a pharmaceutical composition comprising a therapeutically effective amount of a composition comprising a nucleic acid sequence encoding one or a plurality of antigens associated with a dysfunctional WNT pathway. In some embodiments, the pharmaceutical composition comprising a therapeutically effective amount of a composition comprising a nucleic acid sequence encoding at least twenty tumor-specific epitopes of the subject. In some embodiments, the methods comprise administering to the subject a plasmid disclosed here and at least one plasmid encoding a cytokine or one or more functional fragments of a cytokine. In some embodiments, the methods comprise administering to the subject a plasmid disclosed here and at elast one plasmid encoding IL-12 or one or more functional fragments of IL-12. In some embodiments, the methods comprise administering to the subject a plasmid disclosed here and at least one plasmid encoding a immunostimulatory agent or one or more a functional fragments of an immunostimulatory agent.

### Immunostimulatory Agents

In some embodiments, the method further comprises administering one or more immunostimulatory agents to the subject. Administration may be either prior to, simultaneously with, or after treatment with the DNA vaccine or immunogenic compositions described herein.

In some embodiments, the present disclosure is directed to the use of immunostimulatory agents, including T cell growth factors and interleukins. Immunostimulatory agents are substances (drugs and nutrients) that stimulate the immune system by inducing activation or increasing activity of any of its components. Immunostimulants include bacterial vaccines, colony stimulating factors, interferons, interleukins, other immunostimulants, therapeutic vaccines, vaccine combinations and viral vaccines.

T cell growth factors are proteins which stimulate the proliferation of T cells. Examples of T cell growth factors include IL-2, IL-7, IL-15, IL-17, IL-21 and IL-33.

Interleukins are a group of cytokines that were first seen to be expressed by white blood cells. The function of the immune system depends in a large part on interleukins, and rare deficiencies of a number of them have been described, all featuring autoimmune diseases or immune deficiency. The majority of interleukins are synthesized by helper CD4 T lymphocytes, as well as through monocytes, macrophages, and endothelial cells. They promote the development and differentiation of T and B lymphocytes, and hematopoietic cells. Examples of interleukins include IL-1, IL-2, IL-3, IL-4, IL-5, IL-6, IL-7, IL-8, IL-9, IL-10, IL-11, IL-12, IL-13, IL-14, IL-15 and IL-17. In some embodiments, the interleukin is IL-12.

In some embodiments, the DNA plasmids are delivered with immunostimulatory agents that are genes for proteins which further enhance the immune response against such target proteins. Examples of such genes are those which encode other cytokines and lymphokines such as alpha-interferon, gamma-interferon, platelet derived growth factor (PDGF), TNFα, TNFβ, GM-CSF, epidermal growth factor (EGF), IL-1, IL-2, IL-4, IL-5, IL-6, IL-10, IL-12, IL-18, MHC, CD80, CD86 and IL-15 including IL-15 having the signal sequence deleted and optionally including the signal peptide from IgE. Other genes which may be useful include those encoding: MCP-1, MIP-1α, MIP-1p, IL-8, RANTES, L-selectin, P-selectin, E-selectin, CD34, GlyCAM-1, MadCAM-1, LFA-1, VLA-1, Mac-1, p150.95, PECAM, ICAM-1, ICAM-2, ICAM-3, CD2, LFA-3, M-CSF, G-CSF, IL-4, mutant forms of IL-18, CD40, CD40L, vascular growth factor, fibroblast growth factor, IL-7, nerve growth factor, vascular endothelial growth factor, Fas, TNF receptor, Fit, Apo-1, p55, WSL-1, DR3, TRAMP, Apo-3, AIR, LARD, NGRF, DR4, DR5, KILLER, TRAIL-R2, TRICK2, DR6, Caspase ICE, Fos, c-jun, Sp-1, Ap-1, Ap-2, p38, p65Rel, MyD88, IRAK, TRAF6, IkB, Inactive NIK, SAP K, SAP-1, JNK, interferon response genes, NFkB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP1, TAP2 and functional fragments thereof.

A combination of any one or more (e.g. 1, 2, 3, 4, 5 or more) immunostimulatory agents can be used in combination with the DNA vaccine or immunogenic compositions described herein.

### Methods Comprising Chemotherapeutic Agents

In a further embodiment, the method further comprises administering a chemotherapeutic agent, targeted therapy or radiation to the subject. Administration may be either prior to, simultaneously with, or after treatment with the DNA vaccine or immunogenic compositions described herein.

Examples of cancer therapeutic agents or chemotherapeutic agents include alkylating agents such as thiotepa and cyclophosphamide (CYTOXAN); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; antimetabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2, 2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel (TAXOL™, Bristol-Myers Squibb Oncology, Princeton, N.J.) and doxetaxel (TAXOTEPvE™, Pvhne-Poulenc Rorer, Antony, France); chlorambucil; gemcitabine; 6-thioguanine; mercaptopurine; methotrexate; platinum analogs such as cisplatin and carboplatin; vinblastine; trastuzumab, docetaxel, platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-1 1; topoisomerase inhibitor RFS 2000; difluoromethylomithine (DMFO); retinoic acid derivatives such as Targretin™ (bexarotene), Panretin™ (alitretinoin); ONTAKT™ (denileukin diftitox); esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4 (5)-imidazoles, 4-hydroxytamoxifen, trioxifene, keoxifene, LY 1 17018, onapristone, and toremifene (Fareston); and anti-androgens such as flutamide, nilutamide, bicalutamide, leuprolide, and goserelin; and pharmaceutically acceptable salts, acids or derivatives of any of the above. Further cancer therapeutic agents include sorafenib and other protein kinase inhibitors such as afatinib, axitinib, bevacizumab, cetuximab, crizotinib, dasatinib, erlotinib, fostamatinib, gefitinib, imatinib, lapatinib, lenvatinib, mubritinib, nilotinib, panitumumab, pazopanib, pegaptanib, ranibizumab, ruxolitinib, trastuzumab, vandetanib, vemurafenib, and sunitinib; sirolimus (rapamycin), everolimus and other mTOR inhibitors.

Examples of additional chemotherapeutic agents include topoisomerase I inhibitors (e.g., irinotecan, topotecan, camptothecin and analogs or metabolites thereof, and doxorubicin); topoisomerase II inhibitors (e.g., etoposide, teniposide, and daunorubicin); alkylating agents (e.g., melphalan, chlorambucil, busulfan, thiotepa, ifosfamide, carmustine, lomustine, semustine, streptozocin, decarbazine, methotrexate, mitomycin C, and cyclophosphamide); DNA intercalators (e.g., cisplatin, oxaliplatin, and carboplatin); DNA intercalators and free radical generators such as bleomycin; and nucleoside mimetics (e.g., 5-fluorouracil, capecitibine, gemcitabine, fludarabine, cytarabine, mercaptopurine, thioguanine, pentostatin, and hydroxyurea). Moreover, exemplary chemotherapeutic agents that disrupt cell replication include: paclitaxel, docetaxel, and related analogs; vincristine, vinblastin, and related analogs; thalidomide, lenalidomide, and related analogs (e.g., CC-5013 and CC-4047); protein tyrosine kinase inhibitors (e.g., imatinib mesylate and gefitinib); proteasome inhibitors (e.g., bortezomib); NF-κB inhibitors, including inhibitors of IκB kinase; antibodies which bind to proteins overexpressed in cancers and other inhibitors of proteins or enzymes known to be upregulated, over-expressed or activated in cancers, the inhibition of which downregulates cell replication.

A combination of any one or more (e.g. 1, 2, 3, 4, 5 or more) chemotherapeutic agents can be used in combination with the DNA vaccine or immunogenic compositions described herein.

In some embodiments, the chemotherapeutic agent and the DNA vaccine or immunogenic compositions are administered substantially simultaneously. Substantially simultaneous administration can be accomplished, for example, by administering to the subject a single capsule having a fixed ratio of each therapeutic agent or in multiple, single capsules for each of the therapeutic agents, or, in some embodiment, substantially simultaneously mean that one vaccine is administered sequentially before or after a vaccine adjuvant or chemotherapeutic agent but within 1 minute or 60 mins of receiving the second or third pharmaceutical composition. For example, one combination of the present disclosure may comprise a pooled sample of tumor specific neoantigens and a checkpoint inhibitor administered at the same or different times, or the)′ can be formulated as a single, co-formulated pharmaceutical composition comprising the two compounds. As another example, a combination of the present disclosure (e.g., DNA neoantigen vaccines and a checkpoint inhibitor) may be formulated as separate pharmaceutical compositions that can be administered at the same or different time. As used herein, the term “simultaneously” is meant to refer to administration of one or more agents at the same time. For example, in some embodiments, a cancer vaccine or immunogenic composition and a checkpoint inhibitor are administered simultaneously). Simultaneously includes administration contemporaneously, that is during the same period of time. In some embodiments, the one or more agents are administered simultaneously in the same hour, or simultaneously in the same day. Sequential or substantially simultaneous administration of each therapeutic agent can be effected by any appropriate route including, but not limited to, oral routes, intravenous routes, sub-cutaneous routes, intramuscular routes, direct absorption through mucous membrane tissues (e.g., nasal, mouth, vaginal, and rectal), and ocular routes (e.g., intravitreal, intraocular, etc.). The therapeutic agents can be administered by the same route or by different routes. For example, one component of a particular combination may be administered by intravenous injection while the other component(s) of the combination may be administered orally. The components may be administered in any therapeutically effective sequence. A “combination” embraces groups of compounds or non-drug therapies useful as part of a combination therapy.

In some embodiments, methods of treating, methods of enhancing an immune response, methods of reducing tolerance to checkpoint inhibitors or methods of reducing resistance to checkpoint inhibitors further comprise a step of administering Pembrolizumab. In some embodiments, methods of treating, methods of enhancing an immune response, methods of reducing tolerance to checkpoint inhibitors or methods of reducing resistance to checkpoint inhibitors further comprise a step of administering Nivolumab.

In some embodiments, methods of treating, methods of enhancing an immune response, methods of reducing tolerance to checkpoint inhibitors or methods of reducing resistance to checkpoint inhibitors further comprise a step of administering Cemiplimab.

In some embodiments, methods of treating, methods of enhancing an immune response, methods of reducing tolerance to checkpoint inhibitors or methods of reducing resistance to checkpoint inhibitors further comprise a step of administering Dostaslimab. In some embodiments, methods of treating, methods of enhancing an immune response, methods of reducing tolerance to checkpoint inhibitors or methods of reducing resistance to checkpoint inhibitors further comprise a step of administering Atezolizumab. In some embodiments, methods of treating, methods of enhancing an immune response, methods of reducing tolerance to checkpoint inhibitors or methods of reducing resistance to checkpoint inhibitors further comprise a step of administering Avelumab.

In some embodiments, methods of treating, methods of enhancing an immune response, methods of reducing tolerance to checkpoint inhibitors or methods of reducing resistance to checkpoint inhibitors further comprise a step of administering Durvalumab. In some embodiments, methods of treating, methods of enhancing an immune response, methods of reducing tolerance to checkpoint inhibitors or methods of reducing resistance to checkpoint inhibitors further comprise a step of administering Ipilimumab.

In some embodiments, methods of treating, methods of enhancing an immune response, methods of reducing tolerance to checkpoint inhibitors or methods of reducing resistance to checkpoint inhibitors further comprise a step of administering Relatlimab.

In some embodiments, methods of treating, methods of enhancing an immune response, methods of reducing tolerance to checkpoint inhibitors or methods of reducing resistance to checkpoint inhibitors further comprise a step of administering Spartalizumab.

In some embodiments, methods of treating, methods of enhancing an immune response, methods of reducing tolerance to checkpoint inhibitors or methods of reducing resistance to checkpoint inhibitors further comprise a step of administering Camrelizumab.

In some embodiments, methods of treating, methods of enhancing an immune response, methods of reducing tolerance to checkpoint inhibitors or methods of reducing resistance to checkpoint inhibitors further comprise a step of administering Sintilimab.

In some embodiments, methods of treating, methods of enhancing an immune response, methods of reducing tolerance to checkpoint inhibitors or methods of reducing resistance to checkpoint inhibitors further comprise a step of administering Tislelizumab

In some embodiments, methods of treating, methods of enhancing an immune response, methods of reducing tolerance to checkpoint inhibitors or methods of reducing resistance to checkpoint inhibitors further comprise a step of administering Toripalimab.

In some embodiments, methods of treating, methods of enhancing an immune response, methods of reducing tolerance to checkpoint inhibitors or methods of reducing resistance to checkpoint inhibitors further comprise a step of administering Tremelimumab.

In certain embodiments, the subject nucleic acid molecules, and compositions comprising the nucleic acid molecules, of the disclosure can be used alone.

### Vaccines

In an exemplary embodiment, the present disclosure is directed to an immunogenic composition, e.g., a vaccine, composition comprising the nucleic acid molecules described herein, capable of eliciting an immune response, and in particular a specific T-cell response.

DNA vaccines are described in U.S. Pat. Nos. 5,593,972, 5,739,118, 5,817,637, 5,830,876, 5,962,428, 5,981,505, 5,580,859, 5,703,055, 5,676,594, and the priority applications cited therein, which are each incorporated herein by reference. In addition to the delivery protocols described in those applications, alternative methods of delivering DNA are described in U.S. Pat. Nos. 4,945,050 and 5,036,006, which are both incorporated herein by reference.

In certain embodiments, the vaccine composition comprises mutant neo-antigenic nucleic acid molecules as described herein (e.g. comprising a nucleic acid sequence comprising the formula: [(antigen expression domain 1)-(linker)-(antigen expression domain 2)-(linker)]n), corresponding to tumor specific neo-antigens identified by the methods described herein. A suitable vaccine will preferably contain a plurality of tumor specific neo-antigenic nucleic acid molecules. In some embodiments, the vaccine comprises from about 1 to about 200 nucleic acid sequences that encode neoantigens or neoantigenic epitopes. In some embodiments, the neoantigenic epitopes are from about 8 to about 15 amino acids in length encoded by a nucleic acid sequence.

In some embodiments, the vaccine comprises from about 2 to about 100, from about 2 to about 58, from about 2 to about 29, or over 20 nucleic acid sequences that encode neoantigens or neoantigenic epitopes. In certain embodiments, the vaccine will include about 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 nucleic acid sequences that encode neoantigens or neoantigenic epitopes.

In certain embodiments, the vaccine will include about 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleic acid sequences that encode neoantigens or neoantigenic epitopes.

In certain embodiments, the vaccine will include about 41, 42, 43, 44, 45, 46, 47, 48, 49, 50, 51, 52, 53, 54, 55, 56, 57, 58, 59, 60, 61, 62, 63, 64, 65, 66, 67, 68, 69 or 70 nucleic acid sequences that encode neoantigens or neoantigenic epitopes.

In certain embodiments, the vaccine will include about 61, 62, 63, 64, 65, 66, 67, 68, 69, 70, 71, 72, 73,74, 75, 76, 77, 78, 79, 80, 81, 82, 83, 84, 85, 86, 87, 88, 89 or 90 nucleic acid sequences that encode neoantigens or neoantigenic epitopes.

In certain embodiments, the vaccine will include about 81, 82, 83, 84, 85, 86, 87, 88, 89, 90, 91, 92, 93, 94, 95, 96, 97, 98, 99 or 100 nucleic acid sequences that encode neoantigens or neoantigenic epitopes.

In certain embodiments, the vaccine composition is capable of enhancing a CD8+ T cell immune response in a subject. In some embodiments, enhancing the CD8+ T cell immune response comprises activating CD8+ T cells. In another embodiment, enhancing the CD8+ T cell immune response comprises expanding CD8+ T cells. In other embodiments, the vaccine composition is capable of raising a specific cytotoxic T-cells response and/or a specific helper T-cell response.

The vaccine composition can further comprise an adjuvant and/or a carrier.

Adjuvants are described herein and are any substance whose admixture into the vaccine composition increases or otherwise modifies the immune response to the mutant peptide. Carriers are scaffold structures, for example a polypeptide or a polysaccharide, to which the neo-antigenic peptides, is capable of being associated. Optionally, adjuvants are conjugated covalently or non-covalently to the peptides or polypeptides of the disclosure.

The ability of an adjuvant to increase the immune response to an antigen is typically manifested by a significant increase in immune-mediated reaction, or reduction in disease symptoms. For example, an increase in humoral immunity is typically manifested by a significant increase in the titer of antibodies raised to the antigen, and an increase in T-cell activity is typically manifested in increased cell proliferation, or cellular cytotoxicity, or cytokine secretion. An adjuvant may also alter an immune response, for example, by changing a primarily humoral or Th2 response into a primarily cellular, or Th1 response. Suitable adjuvants are described herein.

A vaccine composition according to the present disclosure may comprise more than one different adjuvant. Furthermore, the disclosure encompasses a therapeutic composition comprising any adjuvant substance including any of the above or combinations thereof. It is also contemplated that the nucleic acid molecule, and the adjuvant can be administered separately in any appropriate sequence.

Cytotoxic T-cells (CTLs) recognize an antigen in the form of a peptide bound to an MHC molecule rather than the intact foreign antigen itself. The MHC molecule itself is located at the cell surface of an antigen presenting cell. Thus, an activation of CTLs is only possible if a trimeric complex of peptide antigen, MHC molecule, and APC is present. Therefore, in some embodiments the vaccine composition according to the present disclosure additionally contains at least one antigen-presenting cell.

The antigen-presenting cell (or stimulator cell) typically has an MHC class I or II molecule on its surface, and in some embodiments is substantially incapable of itself loading the MHC class I or II molecule with the selected antigen.

Preferably, the antigen-presenting cells are dendritic cells. In some embodiments, the dendritic cells are autologous to a subject. In some embodiments of the present disclosure the antigen presenting cell comprises an expression construct comprising the nucleic acid molecules of the present disclosure. The nucleic acid molecules are capable of transducing the dendritic cell, thus resulting in the presentation of a peptide and induction of immunity.

The disclosure features a method of making an individualized cancer vaccine for a subject suspected of having or diagnosed with a cancer, comprising identifying a plurality of mutations in a sample from the subject; analyzing the plurality of mutations to identify one or more neoantigen mutations; and producing, based on the identified subset, a personalized cancer vaccine.

In some embodiments, identifying comprises sequencing the cancer. Methods for carrying out sequencing are described herein.

In some embodiments, identifying comprises sequencing the cancer.

In some embodiments, analyzing further comprises determining one or more binding characteristics associated with the neoantigen mutation, the binding characteristics selected from the group consisting of binding of the subject-specific peptides to T-cell receptor, binding of the subject-specific peptides to a HLA protein of the subject and binding of the subject-specific peptides to transporter associated with antigen processing (TAP); and ranking, based on the determined characteristics, each of the neo-antigenic mutations.

In some embodiments, the method further comprises cloning nucleic acid sequences encoding the one or plurality of neoantigen mutations into a nucleic acid molecule.

In some embodiments, the nucleic acid molecule is a plasmid. In another embodiment, the nucleic acid molecule comprises a nucleic acid sequence of Formula I that is positioned within the multiple cloning site of a plasmid selected from the group consisting of selected from the group consisting of pGX0001, pGX4501, pGX4503, pGX4504, pGX4505, pGX4506 and pGX6001.

In some embodiments, the nucleic acid sequence of Formula I is positioned with the multiple cloning site of pGX0001. In some embodiments, the nucleic acid sequence of Formula I is positioned with the multiple cloning site of pGX4501. In some embodiments, the nucleic acid sequence of Formula I is positioned with the multiple cloning site of pGX4503.

In some embodiments, the nucleic acid sequence of Formula I is positioned with the multiple cloning site of pGX4504. In some embodiments, the nucleic acid sequence of Formula I is positioned with the multiple cloning site of pGX4505. In some embodiments, the nucleic acid sequence of Formula I is positioned with the multiple cloning site of pGX4506. In some embodiments, the nucleic acid sequence of Formula I is positioned with the multiple cloning site of pGX6001.

In some embodiments, the plasmid is pGX0001. In some embodiments, the plasmid comprises the backbone and linker sequence of pGX0001 comprising a nucleic acid sequence that is an expressible nucleic acid sequence, the expressible nucleic acid sequence comprising at least two or more AEDs that encoding one or more neoantigens from a subject; and wherein at least one neoantigen is an amino acid associated with the WNT pathway.

In some embodiments, the plasmid is pGX4505. In some embodiments, the plasmid comprises the backbone and linker sequence of pGX4505 with at least two or more AED nucleotide sequence encoding one or more neoantigens from a subject.

In some embodiments, the plasmid comprises the backbone and linker sequence of pGX6001 with at least two or more AED nucleotide sequences encoding one or more neoantigens from a subject. In some embodiments, the plasmid comprises at least about 70%, 75%, 80%, 85%, 90%, 91%, 92%, 93%, 94%, 95%, 96%, 97%, 98%, 99% or 100% sequence identity to SEQ ID NO:49, SEQ ID NO:50, SEQ ID NO:51, SEQ ID NO:52, SEQ ID NO:53 and comprises a nucleic acid sequence encoding β-catenin. In some embodiments, the pharmaceutical compositions of the disclosure comprise: (i) a therapeutically effective amount of a nucleic acid molecule comprising at least about 70%, 75%, 80%, 85%, 90%, 91%, 92%, 93%, 94%, 95%, 96%, 97%, 98%, 99% or 100% sequence identity to SEQ ID NO:49, SEQ ID NO:50, SEQ ID NO:51, SEQ ID NO:52, SEQ ID NO:53, wherein the nucleic acid molecule comprises a nucleic acid sequence encoding β-catenin; and (ii) a pharmaceutically acceptable carrier.

### Cells

Disclosed are cells comprising a TCR comprising one alpha and one beta subunit, wherein the alpha and beta subunits are those disclosed in Table 18. In some aspects, the TCR comprising one alpha and one beta subunit comprise one alpha and one beta subunit having at least 70%, 75%, 80%, 85%, 90%, 95%, or 99% sequence identity to the alpha and beta subunits disclosed in Table 18.

Disclosed are cells comprising a TCR comprising one alpha and one beta subunit, wherein an alpha subunit is chosen from one or a combination of amino acid sequences that comprise at least about 70% sequence identity to SEQ ID NOs: 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365; and wherein a beta subunit is chosen from one or a combination of amino acid sequences that comprise at least about 70% sequence identity to SEQ ID NOs: 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362.

### Kits

The present disclosure provides a kit comprising a pharmaceutical composition comprising one or a plurality of nucleic acid molecules as described herein. The components of the kit are preferably formulated in pharmaceutically acceptable carriers.

Also included in the kit are instructions for use in methods of treating cancer in a subject or enhancing a CD8+ T cell immune response in a subject.

The practice of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Wei, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the disclosure, and, as such, may be considered in making and practicing the disclosure.

### CITED REFERENCES

Although the invention is described in detail with reference to specific embodiments thereof, it will be understood that variations which are functionally equivalent are within the scope of this invention. Indeed, various modifications of the invention in addition to those shown and described herein will become apparent to those skilled in the art from the foregoing description and accompanying drawings. Such modifications are intended to fall within the scope of the appended claims. Those skilled in the art will recognize, or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference in their entireties.

The discussion herein provides a better understanding of the nature of the problems confronting the art and should not be construed in any way as an admission as to prior art nor should the citation of any reference herein be construed as an admission that such reference constitutes “prior art” to the instant application.

All references including patent applications and publications cited herein are incorporated herein by reference in their entirety and for all purposes to the same extent as if each individual publication or patent or patent application was specifically and individually indicated to be incorporated by reference in its entirety for all purposes. Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.
Altschul S F, Madden T L, Schäffer A A, Zhang J, Zhang Z, Miller W, Lipman D J. Gapped BLAST and PSI-BLAST: a new generation of protein database search programs. Nucleic Acids Res. 1997 Sep. 1; 25 (17): 3389-402. doi: 10.1093/nar/25.17.3389.
Ausubel et al. (Current Protocols in Molecular Biology, Wiley Interscience, New York, 2001)
Baden et al. J Infect Dis. 2013 Jan. 15; 207 (2): 240-7
Baron et al., 1995, Nucleic Acids Res. 23:3605-06.
Beers M F, Morrisey E E. The Three R's of Lung Health and Disease: Repair, Remodeling, and Regeneration. The Journal of Clinical Investigation. 2011; 121:2065-2073.
Benton and Davis (Science 196:180, 1977);
Berger and Kimmel (Guide to Molecular Cloning Techniques, 1987, Academic Press, New York)
Chen B, Dodge M E, Tang W, Lu J, Ma Z, Fan C W, Wei S, Hao W, Kilgore J, Williams N S, Roth M G, Amatruda J F, Chen C, Lum L. Small Molecule-Mediated Disruption of wnt-Dependent Signaling in Tissue Regeneration and Cancer. Nature Chemical Biology. 2009; 5:100-107.
Current Protocols in Molecular Biology, Ausubel et al., eds., John Wiley & Sons, 1992 Clevers H, Nusse R. Wnt/beta-catenin Signaling and Disease. Cell. 2012; 149:1192-1205
DuPage M, Dooley A L, Jacks T. Conditional Mouse Lung Cancer Models Using Adenoviral or Lentiviral Delivery of cre Recombinase. Nat Protoc. 2009; 4:1064-1072.
Feigner et al., Proc. Natl. Acad. Sci. USA 84:7413-7414 (1987).
Goeddel, 1990, Gene Expression Technology: Methods in Enzymology 185, Academic Press, San Diego, Calif.
Goodman and Gilman's The Pharmacological Basis of Therapeutics, 10th Edition, McGraw-Hill (New York) (2001)
Greaves M, Maley C C. Clonal Evolution in Cancer. Nature. 2012; 481:306-313.
Grunstein and Hogness (Proc. Natl. Acad. Sci., USA 72:3961, 1975).
Harding et al., Clin Cancer Res 2019.
Henikoff et al., Proc. Natl. Acad. Sci. USA, 1992, 89, 10915-10919.
Karlin et al., Proc. Natl. Acad. Sci. USA, 1993, 90, 5873-5787.
Kiken et al, 2011
Komher, J. S. et al., Nucl. Acids. Res. 17:7779-7784 (1989)
Krutzfeldt et al., Nature (Oct. 30, 2005).
Kuppuswamy, M. N. et al., Proc. Natl. Acad. Sci. (U.S.A.) 88:1143-1147 (1991)
Levine et al., 1996, Science 272:1939-1943
Levine et al., 1997, J. Immunol. 159:5921-5930
Lobo N A, Shimono Y, Qian D, Clarke M F. The Biology of Cancer Stem Cells. Annu Rev Cell Dev Biol. 2007; 23:675-699.
Mannino & Gould-Fogerite, BioTechniques 6 (7): 682-691 (1988)
Nyren, P. et al., Anal. Biochem. 208:171-175 (1993)
Prezant, T. R. et al., Hum. Mutat. 1:159-164 (1992)
Sambrook et al., Molecular Cloning: A Laboratory Manual, Second Ed., Cold Spring Harbor (1989).
Schepers A G, Snippert H J, Stange D E, van den Born M, van Es J H, van de Wetering M, Clevers H. Lineage Tracing Reveals lgr5+Stem Cell Activity in Mouse Intestinal Adenomas. Science. 2012; 337:730-735.
Sokolov, B. P., Nucl. Acids Res. 18:3671 (1990)
Soutschek et al., Nature 432:173-178 (2004)
Su et al, 2011
Syvanen, A.-C, et al., Genomics 8:684-692 (1990)
Syvanen, A.-C, et al., Amer. J. Hum. Genet. 52:46-59 (1993)
Tatusova and Madden, FEMS Microbiol Lett., 1999, 174, 247-250
Ugozzoli, L. et al., GATA 9:107-112 (1992)
Valent P, Bonnet D, De Maria R, Lapidot T, Copland M, Melo J V, Chomienne C, Ishikawa F, Schuringa J J, Stassi G, Huntly B, Herrmann H, Soulier J, Roesch A, Schuurhuis G J, Wohrer S, Arock M, Zuber J, Cerny-Reiterer S, Johnsen H E, Andreeff M, Eaves C. Cancer Stem Cell Definitions and Terminology: The Devil is in the Details. Nature Reviews. Cancer. 2012; 12:767-775.
Wolff et al., Science 247:1465-1468 (1990)
Science of Synthesis: Houben-Weyl Methods of Molecular Transformation. Georg Thieme Verlag, Stuttgart, Germany. 2005.
J. F. W. McOmie, “Protective Groups in Organic Chemistry”, Plenum Press, London and New York 1973
T. W. Greene and P. G. M. Wuts, “Protective Groups in Organic Synthesis”, Third edition, Wiley, New York 1999
“The Peptides”; Volume 3 (editors: E. Gross and J. Meienhofer), Academic Press, London and New York 1981
Methoden der organischen Chemie” (Methods of Organic Chemistry), Houben Weyl, 4th edition, Volume 15/I, Georg Thieme Verlag, Stuttgart 1974
H.-D. Jakubke and H. Jeschkeit, “Aminosauren, Peptide, Proteine” (Amino acids, Peptides, Proteins), Verlag Chemie, Weinheim, Deerfield Beach, and Basel 1982
Jochen Lehmann, “Chemie der Kohlenhydrate: Monosaccharide and Derivate” (Chemistry of Carbohydrates: Monosaccharides and Derivatives), Georg Thieme Verlag, Stuttgart 1974
French Patent No. 2,650,840
U.S. Pat. No. 4,656,127
U.S. Pat. No. 4,945,050
U.S. Pat. No. 5,036,006
U.S. Pat. No. 5,279,833
U.S. Pat. No. 5,204,253
U.S. Pat. No. 5,580,859
U.S. Pat. No. 5,589,466
U.S. Pat. No. 5,593,972
U.S. Pat. No. 5,676,594
U.S. Pat. No. 5,703,055
U.S. Pat. No. 5,739,118
U.S. Pat. No. 5,817,637
U.S. Pat. No. 5,830,876
U.S. Pat. No. 5,962,428
U.S. Pat. No. 5,981,505
U.S. Pat. No. 7,245,963
U.S. Pat. No. 7,283,337
U.S. Pat. No. 9,981,036
U.S. Pat. No. 9,272,024
U.S. patent application No. 20060252077
U.S. Patent Publication No. 20050107325
U.S. Patent Publication No. 20050052630
U.S. Patent Publication No. 2005005263
U.S. Patent Publication No. 20050182005
U.S. Patent Publication No. 20040175727
U.S. Patent Publication No. 20020115080
U.S. patent application Ser. No. 11/874,072
U.S. Provisional Application Ser. No. 60/852,149
U.S. Provisional Application Ser. No. 60/978,982
WO 2012/159643
WO 2012/159754
WO 1996/18372
WO 1993/24640
WO 1992/15712
WO 1991/02087
WO 1991/06309
Pharmacol Ther. 2015 February; 146:120-31
Vaccine. 2003 Jun. 2; 21 (19-20): 2461-7
Clin Immunol. 2000 January; 94 (1): 64-72
Expert Rev Vaccines. 2007 October; 6 (5): 741-6
Expert Rev Vaccines. 2007 October; 6 (5): 741-6
Vaccine. 2006 Mar. 10; 24 (11): 1958-6
“Oligonucleotide Synthesis” (Gait, 1984)
“Animal Cell Culture” (Freshney, 1987)
“Methods in Enzymology” “Handbook of Experimental Immunology” (Wei, 1996)
“Gene Transfer Vectors for Mammalian Cells” (Miller and Calos, 1987)
“PCR: The Polymerase Chain Reaction”, (Mullis, 1994)
“Current Protocols in Immunology” (Coligan, 1991)

The following Examples are representative of techniques used in carrying out aspects of the present disclosure. It should be appreciated that while these techniques are exemplary of embodiments for the practice of the disclosure, those of skill in the art, in light of the present disclosure, will recognize that numerous modifications can be made without departing from the spirit and intended scope of the disclosure.

### Example 1

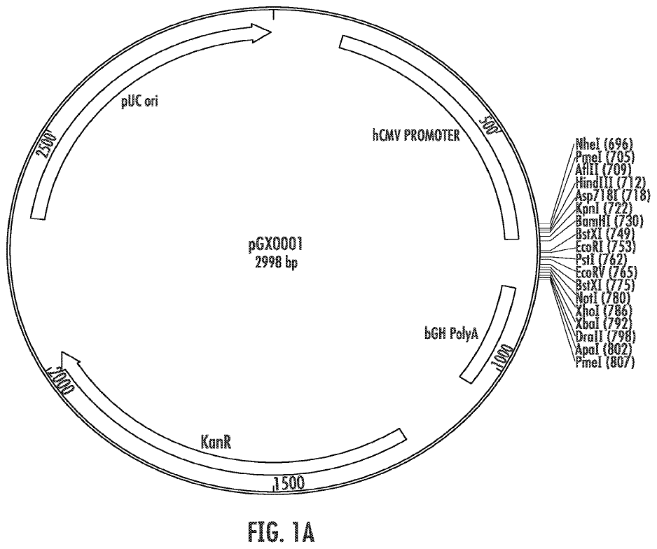

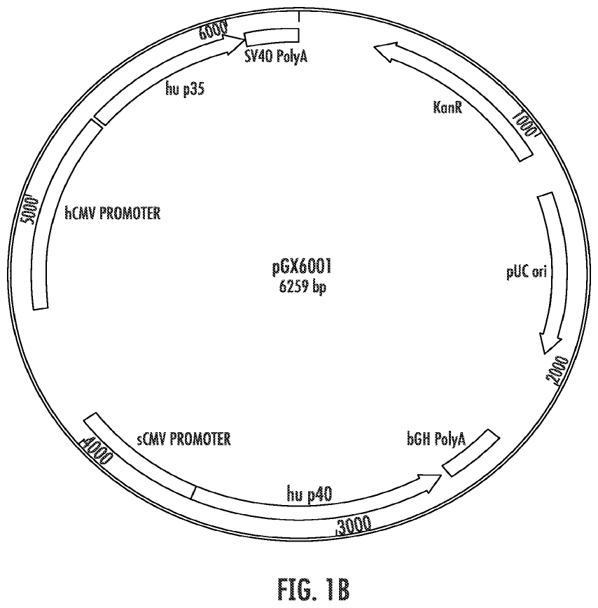

FIG. 1A is a vector diagram of the pGX0001 plasmid. Plasmid sequences of pGX0001 were created comprising nucleic acid constructs that encode the epitopes below. As indicated nucleic acid sequences encoding the amino acid sequence provided were cloned into the pGX0001 vector multiple cloning site.

TABLE 12
Patient PT1

| | Gene | Protein | | SEQ |
|---|---|---|---|---|
| Neo# | Symbol | Variant | Neoantigen Design | ID NO: |
| 1 | CTNNB1 | p.D32G | PDRKAAVSHWQQQSYLGSGIHSGATTTAPSLSG | 70 |
| 2 | TK1 | p.Y181fs | GQSPTLLTSACFFPSAAFTAEFLESLGSLCQHL | 71 |
| 3 | PABPC3 | p.M556K | RLASAPPQKQKQKLGERLFPLIQAMHPTLAGKI | 72 |
| 4 | BEST1 | p.L234V | CGHLYAYDWISIPVVYTQVVTVAVYSFELTCLV | 73 |
| 5 | ABCB11 | p.I420F | DCMSEDGYKLDRIKGEFEFHNVTFHYPSRPEVK | 74 |
| 6 | MAP4K4 | p.S16P | MANDSPAKSLVDIDLPSLRDPAGIFELVEVVGN | 75 |
| 7 | BACH1 | p.L456fs | WLGIRISESPEPGQRTFTTFKFCQLPFYKYSEY | 76 |
| 8 | KIAA1715 | p.L256M | PPLARPILPRERGAMDRIVEYLVGDGPQNRYAL | 77 |
| 9 | DLK1 | p.G118A | TCVSLDDGLYECSCAPAYSGKDCQKKDGPCVIN | 78 |
| 10 | LIG3 | p.F93C | LHVGLCSGPCEMAEQRCCVDYAKRGTAGCKKCK | 79 |
| 11 | INTS6 | p.Y410F | SFESYLKTMPPYFLGPLKKAVRMMGAPNLIADS | 80 |
| 12 | PLXNA2 | p.G304V | PKFHSYVSLPFGCTRAVVEYRLLQAAYLAKPGD | 81 |
| 13 | WDR66 | p.D1029N | GEYVDTGKLIDKINLPNFLKVYLNHKPPFGNTM | 82 |
| 14 | NACC2 | p.K456N | MCTNARRVRKRWLPKINSMLPEGVEMYRTVMGS | 83 |
| 15 | FGFR4 | p.R538fs | PDGPLLPSPGLLRLPGGPRHAVSGVPEVYPPGP | 84 |
| 16 | NRD1 | p.K1062M | VGTQATKYNSEVVDKMIEEFLSSFEEKIENLTE | 85 |
| 17 | SNX12 | p.E76K | TNLPIFKLKESCVRRRYSDFKWLKNELERDSKI | 86 |
| 18 | PIM1 | p.N286K | LIRWCLALRPSDRPTFEEIQKHPWMQDVLLPQE | 87 |
| 19 | PLXNA2 | p.S840N | KFECGWCSGERRCTLHQHCTNPSSPWLDWSSHN | 88 |
| 20 | MAPK1 | p.R194S | HDHTGELTEYVATRWYSAPEIMLNSKGYTKSID | 89 |
| 21 | ZZEF1 | p.I1421L | SSEATEVNPESLAKECLEKSLLLLKELPTGISS | 90 |
| 22 | ATP7A | p.R645L | VKKDRSASHLDHKLEIRQWRRSELVSLFFCIPV | 91 |
| 23 | ENPP2 | p.H31Y | LFTFAVGVNICLGFTAYRIKRAEGWEEGPPTVL | 92 |
| 24 | JOSD2 | p.P9L | MSQAPGAQLSPPTVYHERQRLELCA | 93 |
| 25 | AP3B1 | p.N91 | MSSNSFPYIEQSGGGEATELGQEAT | 94 |
| 26 | MUM1 | p.D570Y | QPCRKMLPDRSRAARYRANQKLVEYIVKAKGAE | 95 |
| 27 | SETDB1 | p.S563C | PVFHGMLERAPAEPCYRAPMEKLFYLPHVCSYT | 96 |
| 28 | CD22 | p.P409S | TYSCVAENILGTGQRGSGAELDVQYPPKKVTTV | 97 |
| 29 | ZFPM2 | p.T416N | SPSATEDSLQPATDLLNRSELPQSQKAMQTKDA | 98 |
| 30 | NPC1L1 | p.Y1122fs | DLRKVPGTDPAFEVFPLHDHQCVL | 99 |
| 31 | CDK12 | p.E814fs | DALDFKKDKGAFYLVFIWTMT | 100 |
| 32 | ITPR2 | p.T1122P | VDNYKQIKADLDQLRLPVEKSELWVEKSSNYEN | 101 |
| 33 | SRPX | p.D278E | NAPENGYMKCSSEGDNYGATCEFSCIGGYELQG | 102 |
| 34 | FGFR4 | p.R397H | ESGSSGKSSSSLVRGVHLSSSGPALLAGLVSLD | 103 |
| 35 | GMIP | p.M130T | WTEKRASYELEFAKSTTKIAEAGKVSIQQQSHM | 104 |
| 36 | PXK | p.A480T | KILARKKSKRSALENSEEHSTKYSNSNNSAGSG | 105 |
| 37 | HTATIP2 | p.P29L | AAAAAALAAALLLLRREDPGLGAGPSMAETEAL | 106 |
| 38 | MED13 | p.M1572V | LPSFPPFGSMNSNAAGSVSTQANTVQSGQLGGQ | 107 |
| 39 | DHX38 | p.D400H | SGVVHRLEVDEDFEEHNAAKVHLMVHNLVPPEL | 108 |
| 40 | CSPP1 | p.I743V | LQIKQQELYKNFLRFQVEEKKQREEAERERLRI | 109 |

TABLE 13
Patient PT6

| | Gene | Protein | | SEQ ID |
|---|---|---|---|---|
| Neo# | Symbol | Variant | Neoantigen Design | NO: |
| 1 | AXIN1 | G342fs | EQQSLSSDADTLSLTDSSVDPPIQDP | 110 |
| 2 | PRKCI | R483H | DQNTEDYLFQVILEKQIRIPHSLSVKAASVLKS | 111 |
| 3 | WDPCP | K739R | LREDGREQEIRDGGSLRMIHFGLV | 112 |
| 4 | SYDE2 | P1043R | LLQLWPVQRLTVKKSTDNLFREQKSSLNYLRQK | 113 |
| 5 | MON2 | V292M | QWLRAVAVESIHRFCMQPQLLRSFCQSYDMKQH | 114 |
| 6 | GBF1 | I541V | LEAIVQLWRIPSFVTELYVNYDCDYYCSNLFEE | 115 |
| 7 | PLXNA1 | N1031H | TPPGQSPGSAPIIINIHRAQLTNPEVKYNYTED | 116 |
| 8 | FAM222B | A151V | PYATVAPSTLAHPQAQVLARQQALQHAQTLAHA | 117 |
| 9 | MGAT1 | T88P | QRGLLQQIGDALSSQRGRVPPAAPPAQPRVPVT | 118 |
| 10 | SMAD6 | R17G | MFRSKRSGLVRRLWRSGVVPDREEGGSGGGGGG | 119 |
| 11 | SUGP1 | I192fs | LEIKVSPPEGAETRKVREIGPLCGRRRPRVRKS | 120 |
| 12 | PRR14 | T194M | TPNFIIPAQRAEPMRIVRQPMPPPGDLEPPFQP | 121 |
| 13 | MTDH | W71fs | GTGALGLLLLFLLGYGLRRRPQKAEEPAPQAGG | 122 |
| 14 | CEP162 | E1182K | DSHVSEVLQENYRLKNKLEGLISEKNELKMKSE | 123 |
| 15 | RBMS1 | Y312C | VSAYQVQSPSWMQPQPCILQHPGAVLTPSMEHT | 124 |
| 16 | KALRN | D2709Y | GRFSIVKKCIHKATRKYVAVKFVSKKMKKKEQA | 125 |
| 17 | VPS13B | Q1628R | GILRDPGSEIEDRQYRIDLQSINIGTAQWHQLK | 126 |
| 18 | LRRN1 | S538Y | DGTQVLKIYVKQTESHYILVSWKVNSNVMTSNL | 127 |
| 19 | MAP3K2 | S182L | PPGYIPDELHQVARNGLFTSINSEGEFIPESMD | 128 |
| 20 | AAR2 | V63M | PKFRGVKMIPPGIHFLHYSSMDKANPKEVGPRM | 129 |
| 21 | PROS1 | N513S | NVSSAEGWHVNVTLSIRPSTGTGVMLALVSGNN | 130 |
| 22 | ZFHX3 | T1450A | CQRSFRTFQALKKHLEASHLELSEADIQQLYGG | 131 |
| 23 | OBSCN | P895L | DSVDERLRVSEPKAVFAKEQLACREVQAEVGAS | 132 |
| 24 | SACS | N2141K | DIKDGRFPYGSTQDYLKPIILIKLVQLGMAKDD | 133 |
| 25 | ZNF91 | Y64F | ENYRNLAFLGIALSKPDLITFLEQGKEPWNMKQ | 134 |
| 26 | ACBD4 | I89V | WNSLGKMSREEAMSAYVTEMKLVAQKVIDTVPL | 135 |
| 27 | OBSCN | R2308W | TFQCVVSPSDVAVVWFWDGALLQPSEKFAISQS | 136 |
| 28 | UBR1 | C1204Y | HVDLFDLESGEYLCPLCKSLYNTVIPIIPLQPQ | 137 |
| 29 | ANKRD36C | E420K | SEIISKLYIPKRKIISPRSIKDVLPPVEEAVDR | 138 |

TABLE 14
Patient PT2

| | Gene | Protein | | SEQ ID |
|---|---|---|---|---|
| Neo# | Symbol | Variant | Neoantigen Design | NO: |
| 1 | CTNNB1 | p.D32G | PDRKAAVSHWQQQSYLGSGIHSGATTTAPSLSG | 139 |
| 2 | JAK1 | p.L910P | GDNTGEQVAVKSPKPESGGNHIADLKKEIEILR | 140 |
| 3 | DPYSL2 | p.R278L | KDHGVNSFLVYMAFKDLFQLTDCQIYEVLSVIR | 141 |
| 4 | GGH | p.K235T | NTDGKIEFISTMEGYTYPVYGVQWHPEKAPYEW | 142 |
| 5 | ANGPT1 | p.H259D | NNSVLQKQQLELMDTVDNLVNLCTKEGVLLKGG | 143 |
| 6 | KMT2A | p.Q2900H | DSNREKDMGLFEVFSQHLPTTEPVDSSVSSSIS | 144 |
| 7 | ZBTB38 | p.V971 | LKAEVFTEILNYIYSSTVIVKRQETVTDLAAAG | 145 |
| 8 | KIF21B | p.V1363M | HPNNVVSIKYCSHSGLMFSVSTSYIKVWDIRDS | 146 |
| 9 | MUM1 | p.L44R | TKNKRRKEYFLAVQIRSLEEKIKVKSTEVEILE | 147 |
| 10 | DOCK8 | p.M1048K | LVKPQKENEQAEKKNISLAFFLYDLLSLMDRGE | 148 |
| 11 | NUP155 | p.N191S | VDIVILGLSYANLQTGSGVLSDSLSGGMQLLPD | 149 |
| 12 | KIAA1109 | p.P4748L | ITKITRRRHENLPHGVASVKEWENYVTATRNEE | 150 |
| 13 | PLCE1 | p.K82M | AVLKVGWLFPLSEVPNFTLLMDGCGCWRLKEDQ | 151 |
| 14 | CD47 | p.A13V | LVAALLLGSVCCGSAQLLENKTKSVEFTF | 152 |
| 15 | POLA1 | p.R491G | SHVFGTNTSSLELFLMNGKIKGPCWLEVKSPQL | 153 |
| 16 | IRX5 | p.A103E | SPYDHTPGMAGSLGYHPYEAPLGSYPYGDPAYR | 154 |
| 17 | COLQ | p.F6L | MTGSSLSLAHLLIISGLLCYSA | 155 |
| 18 | HOOK2 | p.Q200H | AEEGDELQQRCLDLERHLMLLSEEKQSLAQENA | 156 |
| 19 | UGT8 | p.D385E | TMYHGVPVVGIPLFGDHYETMTRVQAKGMGILL | 157 |
| 20 | MLH3 | p.D835Y | QPASHILNSEKFPFSKYEDCLEQQMPSLRESPM | 158 |
| 21 | HIST1H1B | p.A29V | VEKSPAKKKATKKAVGAGAAKRKATGPPVSELI | 159 |
| 22 | NUP210 | p.L28P | LLAAGPSAAAAKPNIPKVLLPETRATRVNETLE | 160 |
| 23 | PER3 | p.F190V | LAPQDMRVFYAHTARAQLPVWNNWTQRAARYEC | 161 |
| 24 | KIFC2 | p.R806L | PAPLRRPLAVLHAPVPTTALARLSRPQRACPSS | 162 |
| 25 | TRIOBP | p.E1829G | KHWFVLTDSSLKYYRDSTAEGADELDGEIDLRS | 163 |
| 26 | USP53 | p.Q19H | VKFLRKPGGNLGKVYHPGSMLSLAPTKGLLNEP | 164 |
| 27 | PTGFRN | p.A428S | DYQVYLNASKVPGFADDPTELSCRVVDTKSGEA | 165 |
| 28 | ZNF202 | p.F52S | QRDDPVLETSHQNERRSRYQEAASPREALIRLR | 166 |
| 29 | SF3B1 | p.Q1228E | HLLNYVWPNVFETSPHVIEAVMGALEGLRVAIG | 167 |
| 30 | LAMA4 | p.P868fs | SRTSMDDLKAFTSLSLYMKPL | 168 |
| 31 | INVS | p.N478S | CAAYGGYINCMAVLMESNADPNIQDKEGRTALH | 169 |
| 32 | SLC17A1 | p.Y20D | DNRLPPKKVPGFCSFRDGLSELVHCCNVIITAQ | 170 |
| 33 | TASP1 | p.R299H | PYSTAVSTSGCGEHLVHTILARECSHALQAEDA | 171 |
| 34 | SMARCA4 | p.L768fs | GVLKQYQIKGLEWLVSCTTTT | 172 |
| 35 | PRPF4B | p.R237K | SKSPSKRSKSQDQAKKSKSPTLRRRSQEKIGKA | 173 |
| 36 | SLC25A11 | p.R288C | FSLWKGFTPYYACLGPHTVLTFIFLEQMNKAYK | 174 |
| 37 | SULF2 | p.A430T | DSFLVERGKLLHKRDNDKVDTQEENFLPKYQRV | 175 |
| 38 | ZNF516 | p.R544M | FRTYHQMVLHSRVHRRARREMDSDGDRAARARC | 176 |
| 39 | COG5 | p.P303S | LEENINSALDIKVLTQSSQSAVRGGPGRSTMPT | 177 |
| 40 | ANKRD37 | p.H36R | GASVNAPPDPCKQSPVRLAAGSGLACELLWQLQ | 178 |

TABLE 15
Patient PT3

| | Gene | Protein | | SEQ ID |
|---|---|---|---|---|
| Neo# | Symbol | Variant | Neoantigen Design | NO: |
| 1 | CTNNB1 | S45P | QSYLDSGIHSGATTTAPPLSGKGNPEEEDVDTS | 179 |
| 2 | TMEM117 | D178A | WMGDFVTAWMVTDMMLQAKPYPDWGKSARAFWK | 180 |
| 3 | ITGA4 | L42Q | PTGRPYNVDTESAQLYQGPHNTLFGYSVVLHSH | 181 |
| 4 | GOLGB1 | N1103Y | EKLAAEEQFQALVKQMYQTLQDKTNQIDLLQAE | 182 |
| 5 | ATP10D | E427V | NEKMDSIVQCRALNIAVDLGQIQYLESDKTGTL | 183 |
| 6 | TEX10 | A871T | EELPVVGQLLRLLLQHTPLRTHMLTNAILVQQI | 184 |
| 7 | NSUN3 | G258R | NLPLLQIELLRSAIKALRPGRILVYSTCTLSKA | 185 |
| 8 | RPL10 | Y177C | GFQCAAPLLILTNKFCFLSTYVFVSTELTGKEL | 186 |
| 9 | CYSLTR1 | T254N | VTAAFLVSEMPYHIQRNIHLHFLHNETKPCDSV | 187 |
| 10 | RYR1 | R3368K | ARPELLQSHFIPTIGRLRKKAGKVVSEEEQLRL | 188 |
| 11 | EMILIN1 | L560V | DRVDAQDETAAEFTLRVNLTAARLGQLEGLLQA | 189 |
| 12 | EPG5 | A2469T | LVQSRQNLVAEERLSSGILGTIGFGRKSPLSNR | 190 |
| 13 | SEMA3C | V10fs | MAFRTICVLLEYLFVLSV | 191 |
| 14 | MYO1D | K53Q | FEKGRIYTFIGEVVVSVNPYQLLNIYGRDTIEQ | 192 |
| 15 | MST1 | T709A | WPAVFARVSVFVDWIHKVMRLG | 193 |
| 16 | FAM122A | S253Y | VCVSSDTLDGNSSSAGYSCNSPAKVSTTTDSPV | 194 |
| 17 | IGFN1 | P308S | VEDAVVESTELEASAISPRVVVPLAETHCEEQG | 195 |
| 18 | LMTK2 | I363V | AAQPYSNLSNLDVLNQVVRERDTKLPKPQLEQP | 196 |
| 19 | RBM10 | D65E | EVKRSQRLRRGPSSPRRPYQEMEYERRGGRGDR | 197 |
| 20 | PPP1R9B | G114D | ASSLNENVDHSALLKLDTSVSERVSREDSKPAP | 198 |
| 21 | ARL8A | G145fs | CHPGPRDLLLLHLLQRKDNID | 199 |
| 22 | C11orf71 | V1161 | DLLRSVLQQRLIALGGIIAARISV | 200 |
| 23 | DHX9 | R680Q | YTMQKHLEMNPHFGSHQYQILPLHSQIPREEQR | 201 |
| 24 | FASN | I175V | CSSSLMALQNAYQAVHSGQCPAAIVGGINVLLK | 202 |
| 25 | EPC1 | Q644L | FRLNIQGLERTLLGFVSKTLDSASAQFAASALV | 203 |
| 26 | ROBO2 | R630L | INPQGLSDPSPMSDPVLTQDISPPAQGVDHRQV | 204 |
| 27 | ERRFI1 | T119fs | LGPHEEDQVVCGFKKLSEWGLCFHPSTDTHKKL | 205 |

TABLE 16
Patient PT5

| | Gene | Protein | | SEQ ID |
|---|---|---|---|---|
| Neo# | Symbol | Variant | Neoantigen Design | NO: |
| 1 | NFE2L2 | D77G | QEKAFFAQLQLGEETGEFLPIQPAQHIQSETSG | 206 |
| 2 | CTNNB1 | S45F | SYLDSGIHSGATTTAPFLSGKGNPEEEDVDTSQ | 207 |
| 3 | AXL | W173fs | GCCPPGHGSRSRPPAQPACSRAEQDILFLLRSP | 208 |
| 4 | MMP17 | P379S | LVSLQPAQMHRFWRGLSLHLDSVDAVYERTSDH | 209 |
| 5 | CHUK | H432R | IQLPIIQLRKVWAEAVRYVSGLKEDYSRLFQGQ | 210 |
| 6 | MID1 | M484L | IFTGQANVVSEYLLAYLELSLVAFFRLIAFLNL | 211 |
| 7 | CHST7 | R8fs | GRRCIRATPRACRARCATCCVRSSAATSPCCGC | 212 |
| 8 | SLC9A6 | M457L | MAFALAIRDTATYARQLMFSTTLLIVFFTVWVF | 213 |
| 9 | MRPS30 | N23K | WRPLLRGPRLSLHTAAKAAATATETTCQDVAAT | 214 |
| 10 | GCSH | D141V | EALAENPGLVNKSCYEVGWLIKMTLSNPSELDE | 215 |
| 11 | SIPA1L3 | V1170G | PSGSFSTPGSATYGRYKPSPERYTAAPHPLLSL | 216 |
| 12 | SPICE1 | S278N | LQTRLQPEESTETLDSNYVVGHVLNSRKQKQLL | 217 |
| 13 | ITPKB | L881V | LEVSPFFKCHEVIGSSVLFIHDKKEQAKVWMID | 218 |
| 14 | CCDC38 | K536N | VAQPKKKLGRRLVFHSNPPSGNKQQLPLVNETK | 219 |
| 15 | ZNF407 | K882E | VHIRRKHSHQYSYLCEVCKYYTVTKGDMERHCA | 220 |
| 16 | OBSCN | R3159P | CHVGSEETRARVPVHDLHVGITKRLKTMEVLEG | 221 |
| 17 | LPCAT1 | Q322H | VTDYTFEDCQLALAEGHLRLPADTCLLEFARLV | 222 |
| 18 | PCDHGB6 | S153L | EIHLEIFESASAGTRLLLDPATDPDININSIKD | 223 |
| 19 | AOX1 | S787A | VYVSTQFPKYIQDIVAATLKLPANKVMCHVRRV | 224 |
| 20 | MTMR12 | H585R | LTQSKSSPKRGFFREETDRLIKNLLGKRISKLI | 225 |
| 21 | UGGT1 | T1477M | NNMIHQVPIKSLPQEWLWCEMWCDDASKKRAKT | 226 |
| 22 | EHBP1 | D95G | PENIEITVTLFKDPHAEEFEGKEWTFVIENESP | 227 |
| 23 | MAP3K10 | G905S | AASRPRLDPWKLVSFSRTLTISPPSRPDTPESP | 228 |
| 24 | CD40LG | M148R | SSKTTSVLQWAEKGYYTRSNNLVTLENGKQLTV | 229 |
| 25 | TP53BP1 | L1881S | LDWQPRENPFQNLKVLSVSDQQQNFLELWSEIL | 230 |
| 26 | TMEM107 | L126H | ERWECTTYWYIFVFCSAHPAVTEMALFVTVEGL | 231 |
| 27 | EPHB4 | R213G | FYKKCAQLTVNLTRFPETVPGELVVPVAGSCVV | 232 |
| 28 | ARID1A | T2150fs | NNVDLILATPPFSRLEKLYSGEVV | 233 |
| 29 | TECPR1 | C918Y | KTMKDFVRRRCWARKYKLVTSGPWLEVPPIALR | 234 |
| 30 | ASCC2 | A711V | ARRMAFLAKKGYRHDSSTAVVGSPRGHGQSRET | 235 |
| 31 | DDX54 | R834W | WRRAQKLHFLQRGGLKQLSARVRPELKTKQQIL | 236 |
| 32 | CDKN2AIPNL | L75V | SGRLDQLLSLSMVWANHVFLGCSYNKDLLDKVM | 237 |
| 33 | KIAA0753 | D351Y | ELGSLIRQLSLCSVKLYADPSVPDVVIDILQQI | 238 |
| 34 | PCDHB15 | P361T | PELSISSLTSPIPENSTETEVALFRIRDRDSGE | 239 |
| 35 | MKNK1 | V67M | TSELLGEGAYAKVQGAMSLQNGKEYAVKIIEKQ | 240 |
| 36 | ANGPT1 | R391K | DWEGNRAYSQYDKFHIGNEKQNYRLYLKGHTGT | 241 |
| 37 | HERC1 | I672T | LRPKLIEELAATRIVDVSTGDSHCLALSHDNEV | 242 |
| 38 | XRCC5 | V407A | YAYDKRANPQVGAAFPHIKHNYECLVYVQLPFM | 243 |
| 39 | PDE8B | T454A | RRYPSMARIHSMTIEAPIAKDGLRRLSGNEYVF | 245 |
| 40 | CRADD | Q125fs | ISTAVRPTTPTTCSRRWWRPSSVGGSASGSRPP | 246 |

TABLE 17
Patient PT4

| | | Protein | | SEQ ID |
|---|---|---|---|---|
| Neo# | Gene Symbol | Variant | Neoantigen Design | NO: |
| 1 | CTNNB1 | S45Y | QSYLDSGIHSGATTTAPYLSGKGNPEEEDVDTS | 247 |
| 2 | APOB | T1973P | HLVSRKSISAALEHKVSALLPPAEQTGTWKLKT | 248 |
| 3 | FGA | V149L | DLRSRIEVLKRKVIEKLQHIQLLQKNVRAQLVD | 249 |
| 4 | ARHGAP29 | H819R | SKDLLRQLPASNENSLRFLIVHLKRVVDHAEEN | 250 |
| 5 | LRRC40 | R581Q | TLLLDGNPFRVPQAAILMKGTAAILEYLRDRIP | 251 |
| 6 | TRAF2 | L103P | SILESSSAFPDNAARREVESPPAVCPSDGCTWK | 252 |
| 7 | C3orf17 | A247V | GQPYFEAFKKKMPIAFVAKGINKLLNKLFLINE | 253 |
| 8 | ACTN4 | S568A | MEDLQDMFIVHTIEEIEGLIAAHDQFKSTLPDA | 254 |
| 9 | CPS1 | V524E | MGGQTALNCGEELFKRGVLKEYGVKVLGTSVES | 255 |
| 10 | ZFPM2 | S1117T | AKGVNGSSQAPTTGKYCRLCDIQFNNLSNFITH | 256 |
| 11 | CAMTA2 | Y139D | HMKLKVQGMECLDGCYVHSSIVPTFHRRCTWLL | 257 |
| 12 | WBP2 | M1481 | QASRGEVPSGAYGYSYIPSGAYVYPPPVANGMY | 258 |
| 13 | HPD | N202S | YSSLRSIVVANYEESIKMPISEPAPGKKKSQIQ | 259 |
| 14 | ITGA4 | P642H | DLQVSAKIGFLKHHENKTYLAVGSMKTLMLNVS | 260 |
| 15 | ZNF622 | M397R | ELILPSGARVGHRSLRRYYKQRFGLSRAVAVAK | 261 |
| 16 | IPO11 | E1003G | MLGEQGFQSLMETVDTGIVTQLQEFLQGF | 262 |
| 17 | MTOR | R1628H | RQIWWERLQGCQHIVEDWQKILMVRSLVVSPHE | 263 |
| 18 | PSMC4 | R121G | STTGSNYYVRILSTIDGELLKPNASVALHKHSN | 264 |
| 19 | RECK | G515A | ANELCEVNRKGCPSADPCLPYFCVQGCKLGEAS | 265 |
| 20 | DHX38 | G631R | FEDCTSENTLIKYMTDRILLRESLREADLDHYS | 266 |
| 21 | FBXO42 | P173R | WIRPLASGSYPSRKAGATLVVYKDLLVLFGGWT | 267 |
| 22 | MTOR | M938V | SSDYSTSEMLVNVGNLPLDEFYPAVSMVALMRI | 268 |
| 23 | FAM46A | Q72H | CESPTAHCNVLNWEQVHRLDGILSETIPIHGRG | 269 |
| 24 | FEM1B | T582S | NKQNKTPLDKSTSGVSEILLKTQMKMSLKCLAA | 270 |
| 25 | PIEZO2 | Y707H | YVCGGMFFFVSFEGKIVMHKIIYMVLFLFCVAL | 271 |
| 26 | RUSC1 | V739A | LPTPGSWWEQLTQASRAYASGGTEGFPLSRWAP | 272 |
| 27 | PRRC2B | A34T | YSTLSLFDKYKGKSVDTIRSSVIPRHGLQSLGK | 273 |
| 28 | TMEM161B | K150fs | MNISLVWCLLVLSFAIRRWW | 274 |
| 29 | MAFK | R95H | RVELQQEVEKLAHENSSMRLELDALRSKYEALQ | 275 |
| 30 | SYDE2 | R80W | SPQREPRGGQLRTPRMWPSCSRSLESLRVGAKP | 276 |
| 31 | IL17RA | M240V | FTLWNESTHYQILLTSFPHVENHSCFEHMHHIP | 277 |
| 32 | FLNC | G610R | DFVVEAIGTEVGTLGESIERPSQAKIECDDKGD | 278 |
| 33 | SMC4 | I137fs | NVIDSMLFVFGYRAQKNKI | 279 |
| 34 | CDKN2B | A75T | MMGSARVAELLLLHGAEPNCTDPATLTRPVHDA | 280 |
| 35 | RNF43 | P715L | PPGLDKRLLPETLGPCYSNSQPVWLCLTPRQPL | 281 |
| 36 | MPDZ | L88R | SFLLSPNNGNLEATSNIEYAHVPHLSPAVIPTR | 282 |
| 37 | UPF1 | A122T | EEDEEDTYYTKDLPIHTCSYCGIHDPACVVYCN | 283 |
| 38 | C10orf2 | D268G | LISRRDAEVVLTSRELGSLALNQSTGLPTLTLP | 284 |
| 39 | SSH2 | P143T | DFSSNDSSTCTMGLVLTLWSDTLIHLDGDGGFS | 285 |
| 40 | HLF | P133A | SSAAPSVMDLSSRASAALHPGIPSPNCMQSPIR | 286 |

### Example 2

FIG. 1B is a vector diagram of the pGX6001 plasmid. SEQ ID NO:68 and SEQ ID NO: 69 were cloned into pGX6001 as shown in FIG. 1B.

### Example 3

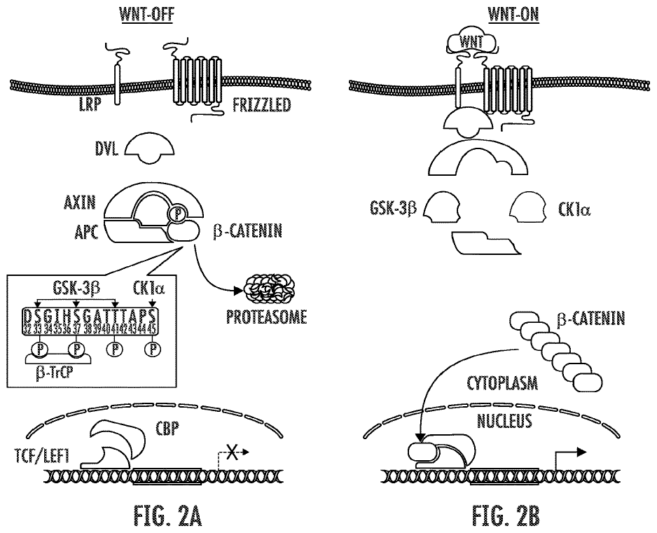

FIG. 2 is a diagram of the Wnt activation pathway. Wnt/β-catenin activation is known to result in resistance to checkpoint inhibitors. Harding et al., Clin Cancer Res 2019. Wnt/β-catenin activation is also known to result in resistance to immunotherapy. www.cbiportal.com.

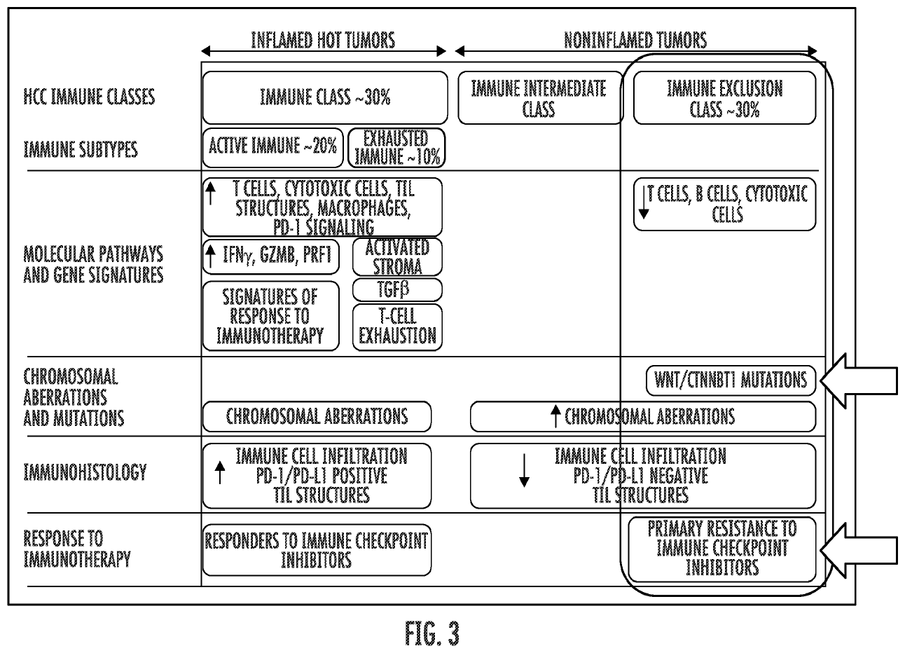

FIG. 3 is a chart classifying immune system involvement in hepatocellular carcinomas. Cancers characterized by mutations in the WNT pathway result in primary resistance to checkpoint inhibitors.

### GNOS-PV02 Prevents Resistance to Anti-PD1 in Wnt Activated HCC

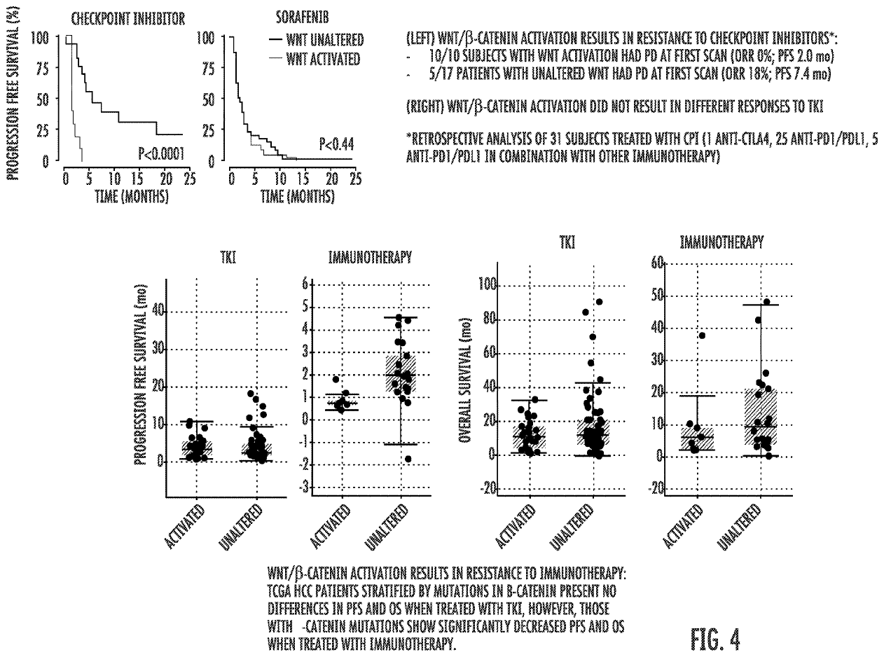

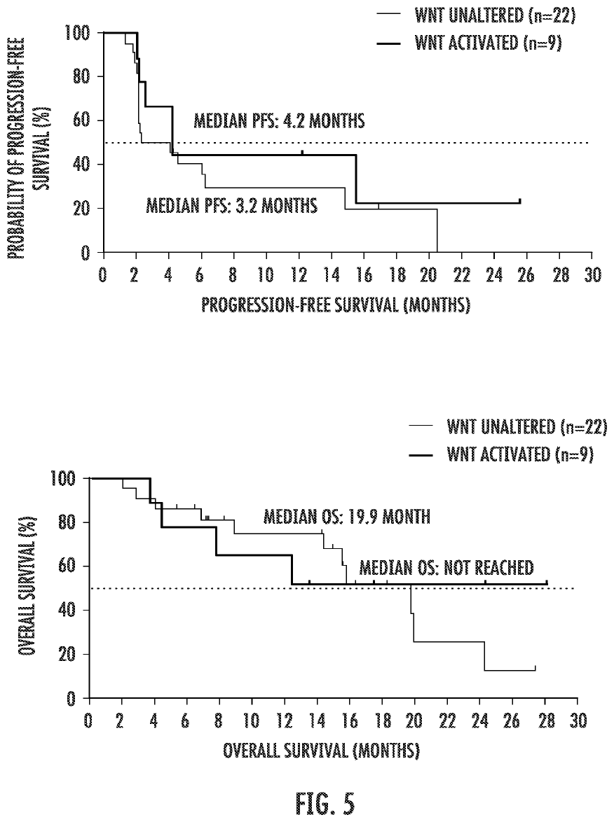

Progression Free Survival. HCC patients were treated with GNOS-PV02 in combination with INO-9012 [encodes IL12] and pembrolizumab in second line treatment following first line treatment with lenvatinib or sorafenib. FIG. 5 shows 19 patients stratified by Wnt/β-catenin pathway status (6 activated and 13 unaltered). Prior PFS data published by Harding et al. in Clin Cancer Res. 2019 (FIG. 4) and data obtained from www.cbioportal.com, demonstrate that only the Wnt/□-catenin unaltered patients are likely to benefit from anti-PD1 immunotherapy. There is a significant difference in progression free survival between the patients with unaltered and altered Wnt/□-catenin pathway. In contrast, as shown in FIG. 5, there is no difference in progression free survival when GNOS-PV02 is present during immunotherapy treatment (pembrolizumab). Wnt/b-catenin activation did not result in resistance to immunotherapy. On the contrary, GNOS-PV02 improved response to checkpoint inhibitors in Wnt activated tumors, which are classically resistant to checkpoint inhibitor treatment. Wnt/b-catenin activation did not result in resistance to anti-PD1 therapy when combined with GNOS-PV02 (PFS 4.2 vs 4.5 months; ORR 50% vs 15.4%; activated vs unaltered, respectively).

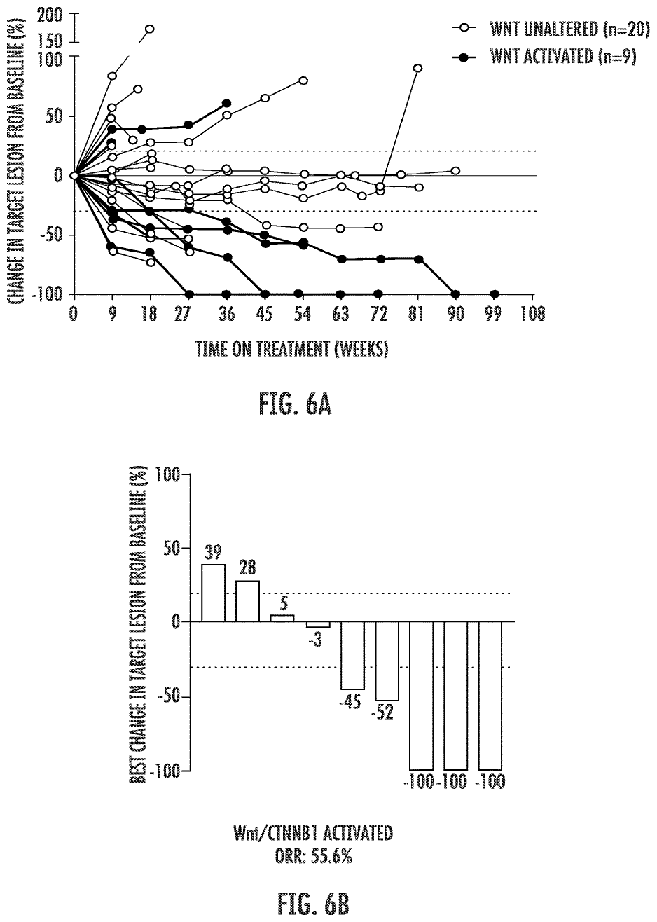

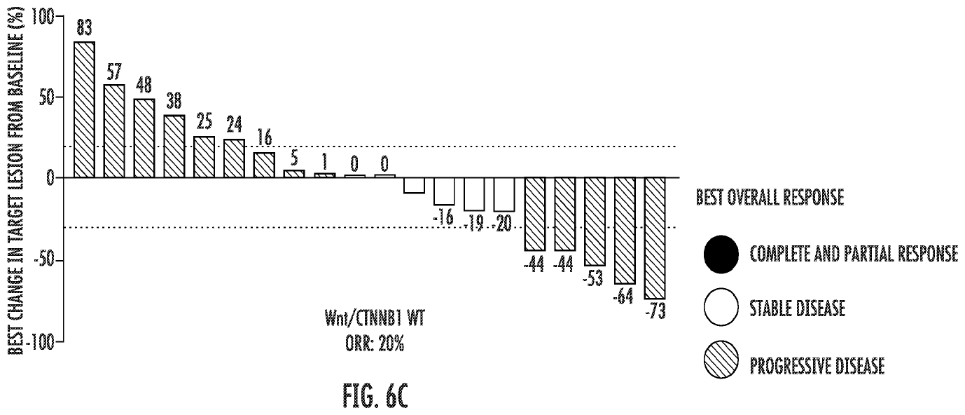

Objective Response Rate (ORR). HCC patients were treated with GNOS-PV02 in combination with INO-9012 and pembrolizumab in second line following first line treatment with lenvatinib or sorafenib. FIG. 6 shows 19 patients stratified by Wnt/β-catenin pathway status (6 activated and 13 unaltered). As opposed to prior PFS data obtained from Harding et al. Clin Cancer Res. 2019 and from www.cbioportal.com, there is no difference in progression free survival when GNOS-PV02 is present during immunotherapy treatment (pembrolizumab). Wnt/b-catenin activation did not result in resistance to immunotherapy in the presence of GNOS-PV02.

FIG. 6A shows the spider plot in which a higher percentage of Wnt pathway activated tumors showed decrease in tumor volume that those with unaltered Wnt pathway.

FIGS. 6B and 6C show the best overall response of patients was significantly higher in patients with Wnt pathway activation (50%) vs unaltered (15.4%). Note that historical data on HCC patients with Wnt pathway activation showed a best overall response of 0%.

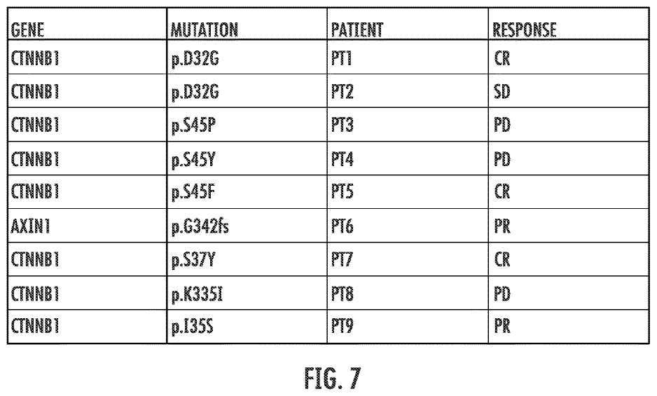

FIG. 7 shows the Wnt pathway genes and corresponding mutations, patient ID and best overall response observed in patients.

Tumors from Subjects with CTNNB1 Activation have More Mutations

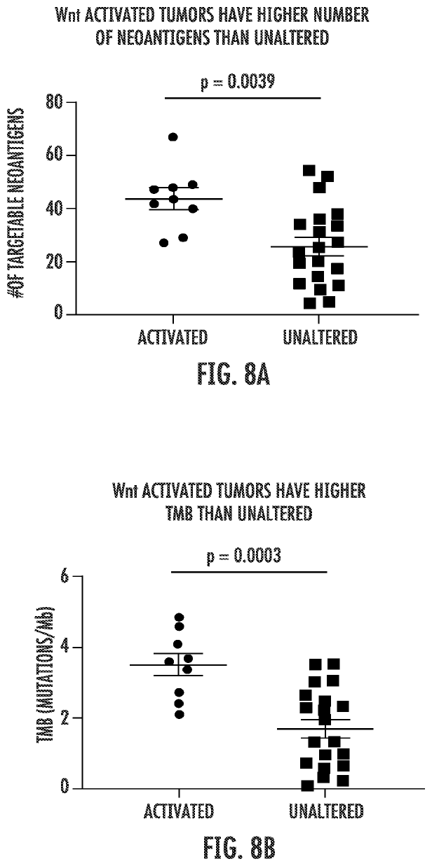

FIG. 8 shows the number of targetable neoantigens and tumor mutational burden measured as mutations per megabase of DNA, resulting from whole exome sequencing, identified in study subjects with Wnt pathway activated or unaltered. FIG. 8A shows subjects with activation in the Wnt pathway presented with a higher number of neoantigens than subjects with an unaltered Wnt pathway. FIG. 8B shows subjects with activation in the Wnt pathway presented a higher tumor mutational burden than those with Wnt pathway unaltered.

Gene Expression in CTNNB1 Mutated Tumors Corresponds with that of Wnt Pathway Activation

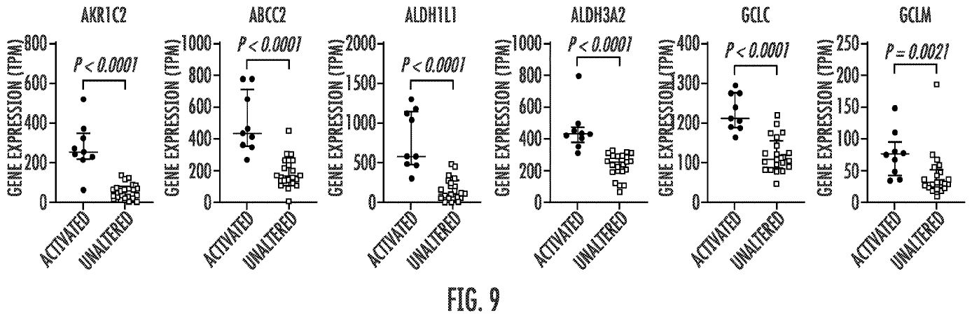

Genes typically associated with Wnt pathway activation are overexpressed in patients with mutations in β-catenin. RNA sequencing was carried out on tumors from [HCC] patients. Wnt pathway activation is classically associated with overexpression of genes such as AKR1C2, ABCC2, ALDH1L1, ALD3A2, GCLC and GCLM. FIG. 9 shows that HCC patients having β-catenin and Axin1 mutations (Activated) overexpress AKR1C2, ABCC2, ALDH1L1, ALD3A2, GCLC and GCLM compared to HCC patients having tumors without β-catenin or Axin1 mutations (Unaltered). Gene expression was measured in RNA transcripts per million (TPM).

### CTNNB1 is Mutated in a Variety of Tumors

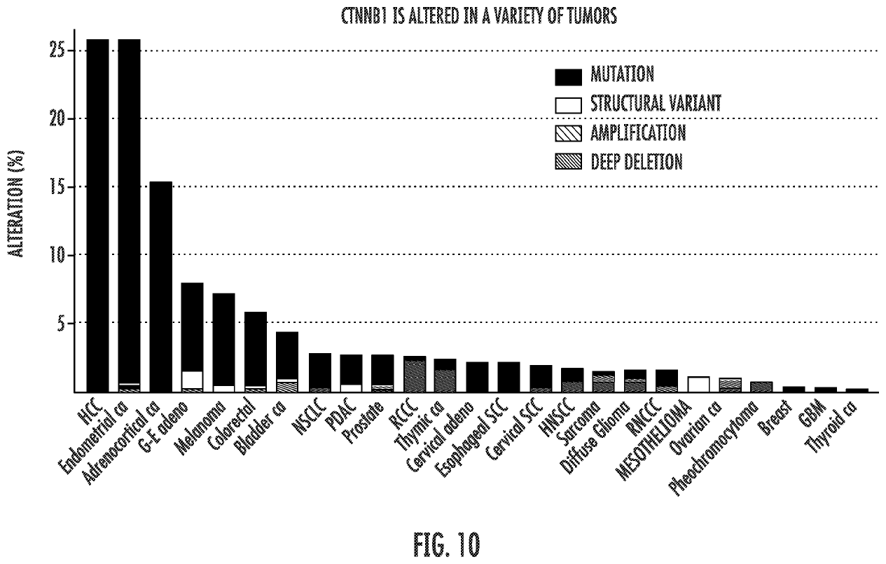

Analysis of frequency of β-catenin gene (CTNNB1) mutations in cancer. CTNNB1 mutations are key mutations driving Wnt pathway activation, describing populations of cancers in which GNOS-PV02 could improve outcomes in response to immunotherapy by combining with checkpoint inhibitors. The CBIO portal database was queried for CTNNB1 mutations (mutation, structural variant, amplification, or deep deletion) in any type of cancer. FIG. 10 shows HCC as the tumor type where CTNNB1 is more commonly mutated (25% of cases), followed by endometrial carcinoma, adrenocortical carcinoma, gastroesophageal carcinoma, melanoma, colorectal carcinoma and bladder cancer.

CTNNB1 Mutated Tumors have Higher Mutations and Less Response to Immunotherapy

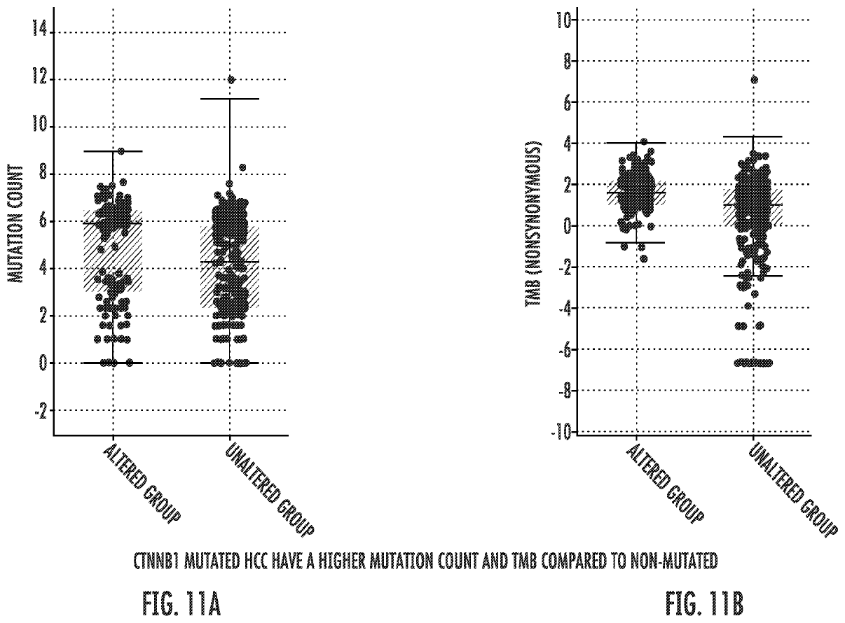

Studies of HCC patients from the CBIO portal database (www.cbioportal.org) were analyzed for the number of mutations and tumor mutational burden (TMB) (mutations per megabase of DNA) in patients with Wnt pathway activated due to CTNNB 1 mutations (Altered group) compared to patients not having CTNNB1 mutations or Wnt pathway activation (Unaltered group). FIG. 11A shows subjects with activation in the Wnt pathway due to CTNNB1 mutations presented a higher number of mutations than those with no CTNNB1 mutations. FIG. 11B shows subjects with activation in the Wnt pathway due to CTNNB1 mutations presented a higher tumor mutational burden than those with no CTNNB1 mutations.

We analyzed responses to therapy in HCC patients, as measured by Progression Free Survival (PFS) in patients treated with either TKI (sorafenib or lenvatinib) or immunotherapy, stratified by status of the β-catenin gene (CTNNB1; Wnt pathway activated or unaltered).

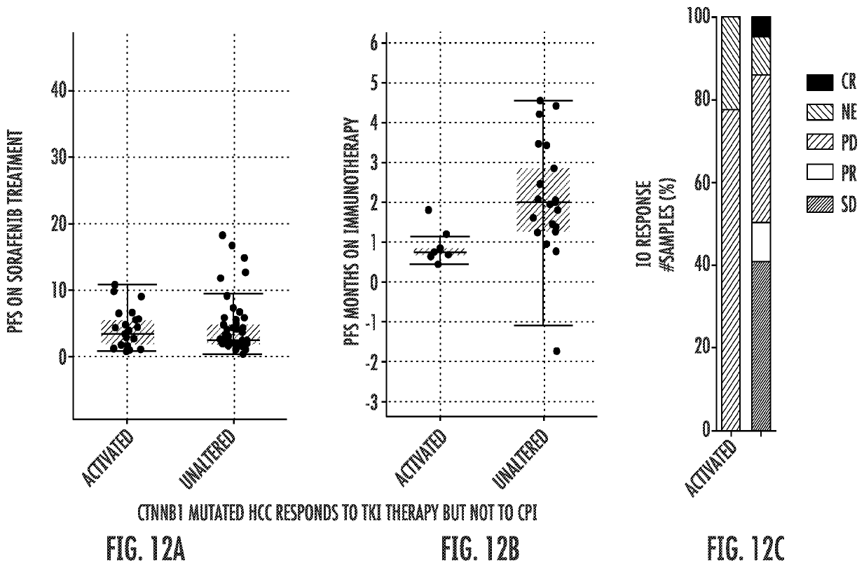

The CBIO portal database (www.cbioportal.org) was queried for studies with HCC patients in which tumor biopsies had been subject to DNA sequencing in which the β-catenin gene could be identified. Data was organized by therapy type (FIG. 12A Sorafenib; FIG. 12B immunotherapy) and Wnt pathway status (activated or unaltered).

FIG. 12A shows that in HCC patients treated with TKI (sorafenib), there was no difference in PFS in patients having a Wnt unaltered pathway compared to patients having Wnt activation. FIG. 12B shows that in HCC patients treated with immunotherapy, there is an increase in PFS in patients having a Wnt unaltered pathway compared to patients having Wnt activation.

Best Overall Responses to immunotherapy in patients with HCC treated with immunotherapy (IO). The best recorded overall response in Wnt activated patients due to CTNNB1 mutation is progressive disease (PD) (80%) and inevaluable (NE) in 20% of subjects. In contrast, CTNNB 1 unaltered subjects' response to immunotherapy presented approximately 5% complete responses (CR), 10% partial responses (PR), 40% stable disease (SD), 35% progressive disease (PD) and 10% inevaluable. FIG. 12C.

This historical data shows that in the absence of GNOS-PV02, HCC patients with Wnt pathway activation due to CTNNB1 mutations respond significantly worse to immunotherapy than Wnt unaltered patients.

Analysis of responses to therapy, measured as overall survival, in HCC patients treated with either TKI (sorafenib or lenvatinib) or immunotherapy, stratified by status of the β-catenin gene (CTNNB1; Wnt pathway activated or unaltered). www.cbioportal.org was queried for HCC studies in which tumor biopsies had been subject to DNA sequencing in which the β-catenin gene could be identified. Data was organized by therapy (Sorafenib or immunotherapy) and Wnt pathway status (activated or unaltered).

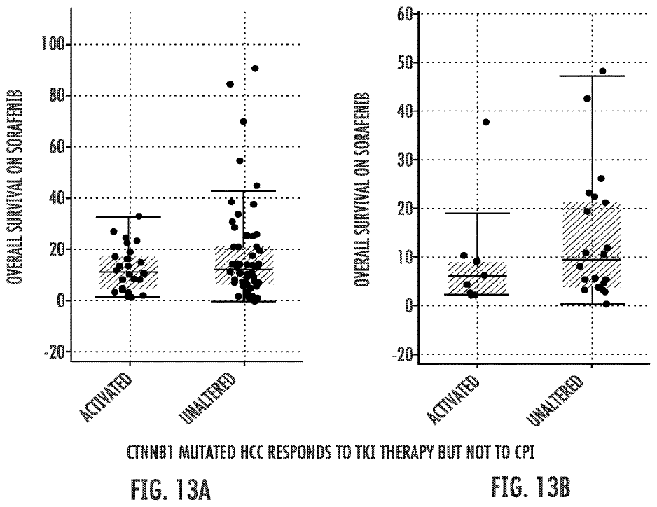

FIG. 13A shows no difference in overall survival was observed in HCC patients treated with TKI with regard to Wnt/β-catenin pathway status. FIG. 13B shows higher overall survival in patients treated with immunotherapy in patients having a Wnt unaltered pathway when compared to patients having Wnt activation.

This historical data shows that, in the absence of GNOS-PV02, HCC patients with Wnt pathway activation due to CTNNB1 mutations respond significantly worse to immunotherapy than Wnt unaltered patients.

### AXIN1 is Mutated in a Variety of Tumors

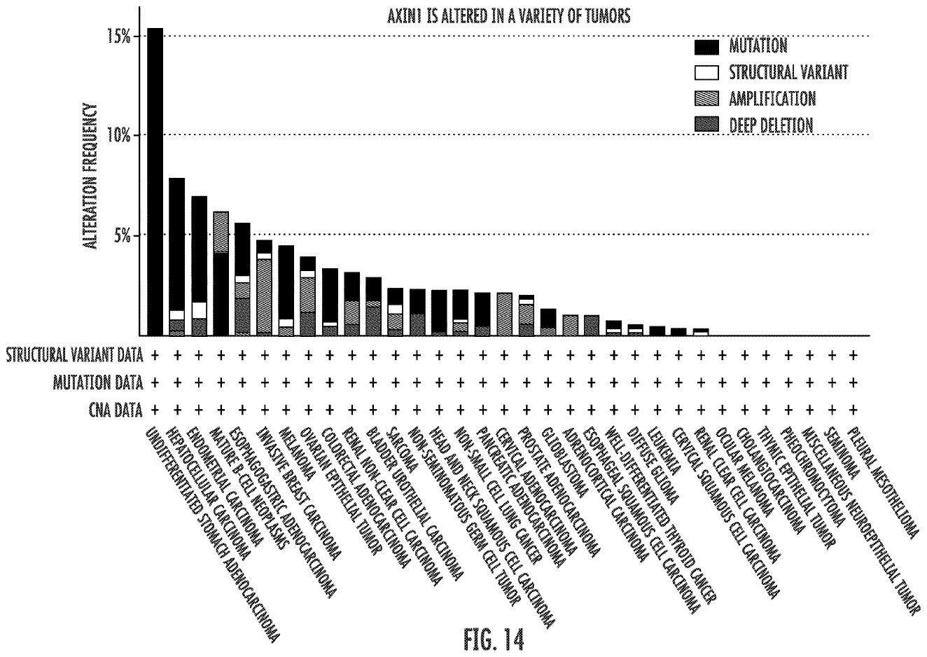

Analysis of frequency of Axin1 gene (AXIN1) mutations in cancer. AXIN1 mutations are key mutations driving Wnt pathway activation, describing populations of cancers in which GNOS-PV02 could improve outcomes in response to immunotherapy by combining with checkpoint inhibitors. The CBIO portal database was queried for AXIN1 mutations (mutation, structural variant, amplification, or deep deletion) in any type of cancer. FIG. 14 shows the frequency of AXIN1 mutations in different types of cancer. Stomach cancer is the tumor type where AXIN1 is more commonly mutated (15% of cases), followed by hepatocellular carcinoma, endometrial carcinoma, mature B-cell neoplasms, esophagogastric carcinoma, invasive breast carcinoma and melanoma.

### Axin2 is Mutated in a Variety of Tumors

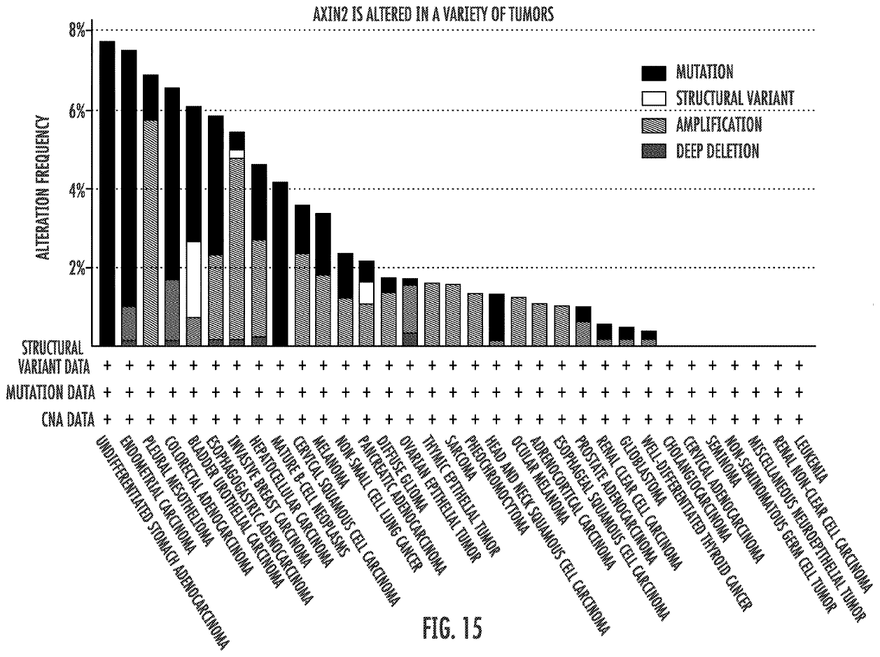

Analysis of frequency of Axin2 gene (AXIN2) mutations in cancer. AXIN2 mutations are key mutations driving Wnt pathway activation, describing populations of cancers in which GNOS-PV02 could improve outcomes in response to immunotherapy by combining with checkpoint inhibitors. The CBIO portal database was queried for AXIN2 mutations (mutation, structural variant, amplification, or deep deletion) in any type of cancer. FIG. 15 shows the frequency of AXIN2 mutations in different types of cancer. The data shows stomach cancer as the tumor type where AXIN2 is more commonly mutated (8% of cases), followed by endometrial carcinoma, pleural mesothelioma, colorectal adenocarcinoma and bladder cancer.

### APC is Mutated in a Variety of Tumors

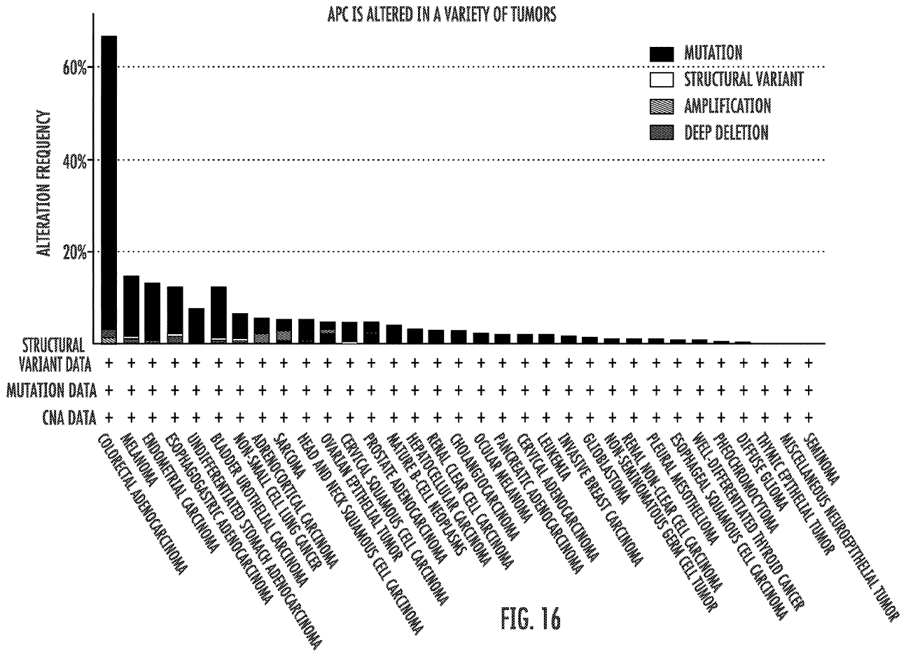

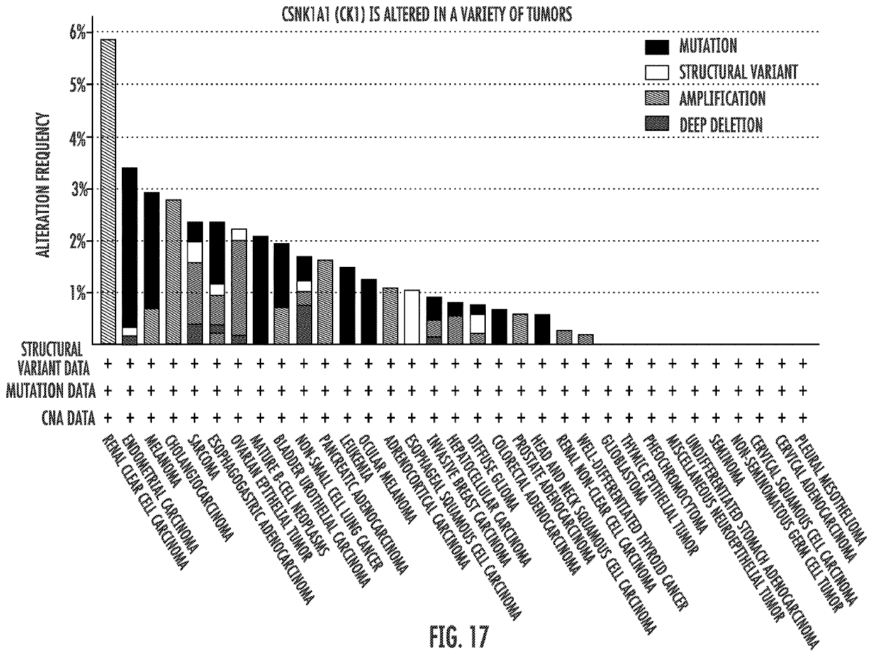

Analysis of frequency of APC gene mutations in cancer. APC mutations are key mutations driving Wnt pathway activation, describing populations of cancers in which GNOS-PV02 could improve outcomes in response to immunotherapy by combining with checkpoint inhibitors. The CBIO portal database was queried for APC mutations (mutation, structural variant, amplification, or deep deletion) in any type of cancer. FIG. 16 shows colorectal adenocarcinoma as the tumor type where APC is more commonly mutated (>60% of cases), followed by melanoma, endometrial carcinoma, esophagogastric carcinoma and stomach cancer.

### CSNK1A1 (CK1) is Mutated in a Variety of Tumors

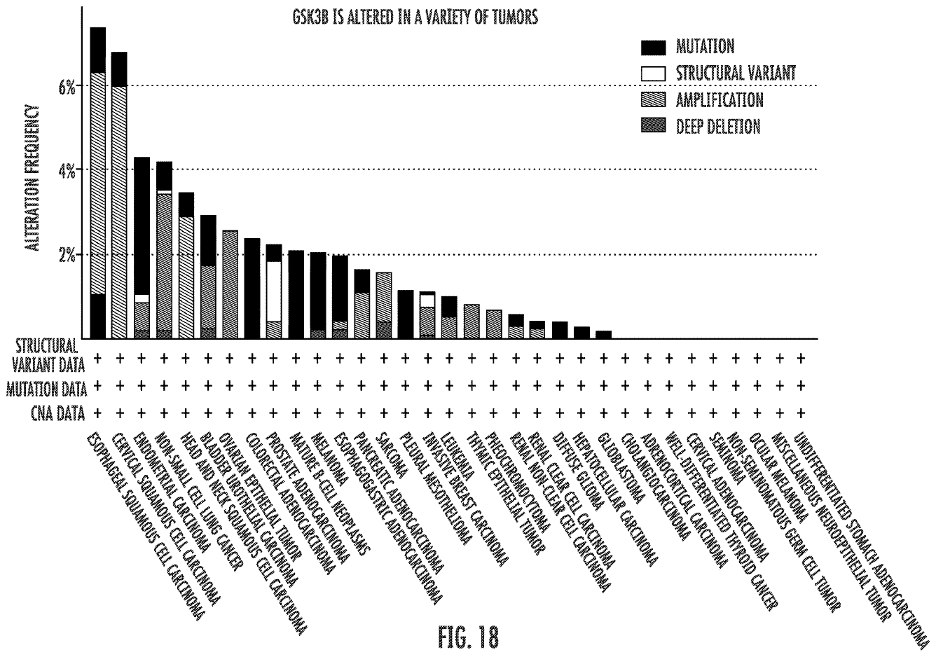

Analysis of frequency of CSNK1A1 gene mutations in cancer. CSNK1A1 mutations are key mutations driving Wnt pathway activation, describing populations of cancers in which GNOS-PV02 could improve outcomes in response to immunotherapy by combining with checkpoint inhibitors. The CBIO portal database was queried for CSNK1A1 mutations (mutation, structural variant, amplification, or deep deletion) in any type of cancer. FIG. 17 shows renal clear cell cancer as the tumor type where CSNK1A1 is more commonly mutated (6% of cases), followed by endometrial carcinoma, melanoma, cholangiocarcinoma and sarcoma.

### GSK3B is Mutated in a Variety of Tumors

Analysis of frequency of GSK3B gene mutations in cancer. GSK3B mutations are key mutations driving Wnt pathway activation, describing populations of cancers in which GNOS-PV02 could improve outcomes in response to immunotherapy by combining with checkpoint inhibitors. The CBIO portal database was queried for GSK3B mutations (mutation, structural variant, amplification, or deep deletion) in any type of cancer. FIG. 18 shows esophageal squamous cell cancer as the tumor type where GSK3B is more commonly mutated (7% of cases), followed by cervical cancer, endometrial carcinoma, non-small cell lung cancer, head and neck cancer, bladder cancer, ovarian cancer, colorectal cancer and prostate cancer.

### Immunology—Patients PT1, PT6, PT2 and PT4

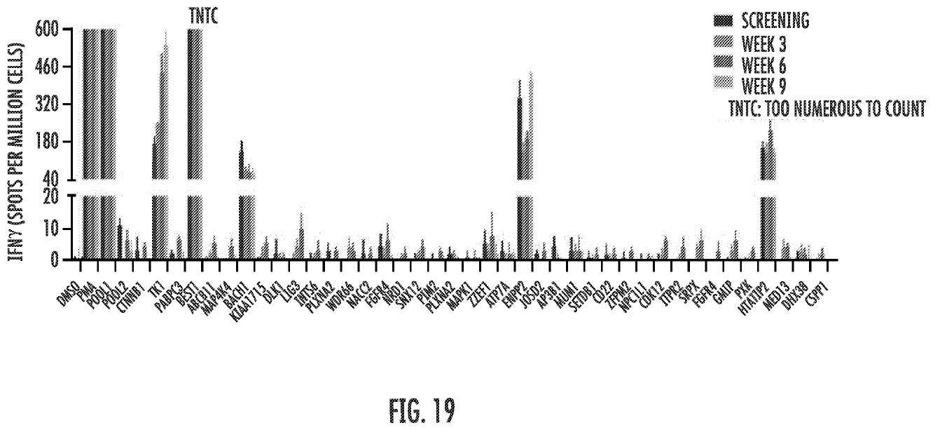

FIG. 19 shows IFNg ELISpot responses to epitopes used in the neoantigen vaccine (GNOS-PV02) generated for PT1. Patient PT1 had strong ELISpot responses detected to several individual dominant epitopes and at multiple time-points relative to background (DMSO) or screening/Day 0. Weaker responses to multiple additional vaccine epitopes were also observed. Responses, although weak, were detected against the CTNNB 1 mutation. Strong immune responses were detected against other neoepitopes included in the vaccine including TK1, BEST1, BACH1, ENPP2 and HTATIP2.

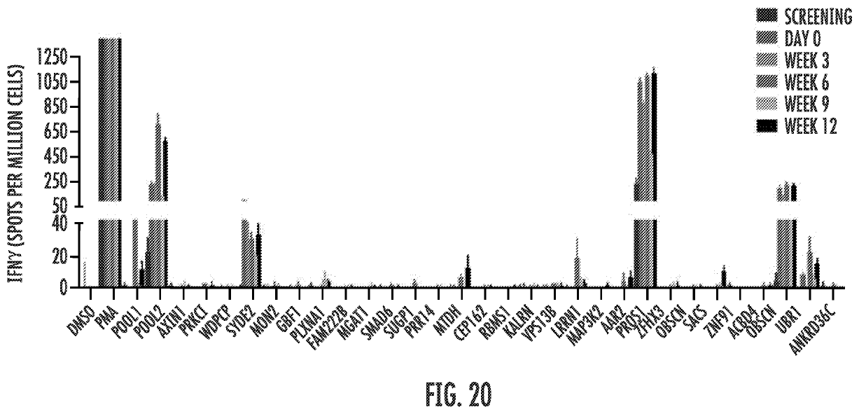

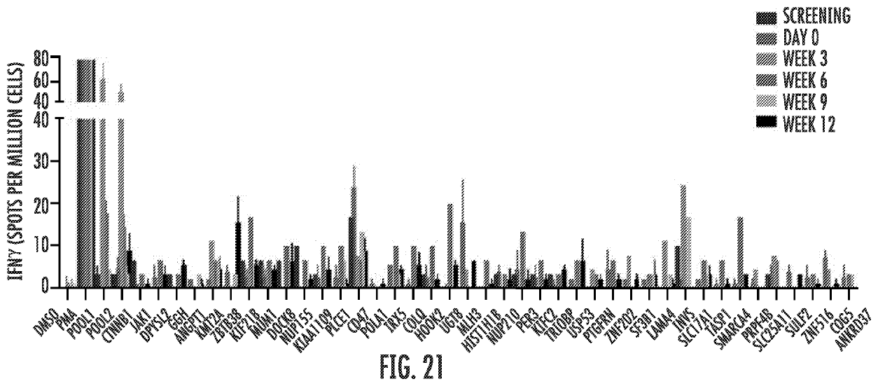

FIG. 20 shows IFNg ELISpot responses to epitopes used in the neoantigen vaccine (GNOS-PV02) generated for PT6. Patient PT6 Strong ELISpot responses detected to multiple individual epitopes (2 dominant epitopes and several weaker epitopes) at multiple time-points relative to background (DMSO) or screening/Day 0. Responses, although weak, were detected against the AXIN1 (Wnt pathway) mutation. Strong immune responses were detected against other neoepitopes included in the vaccine including SYDE2, PROS1, OBSCN or intermediate responses against UBR1.

FIG. 21 shows IFNg ELISpot responses to epitopes used in the neoantigen vaccine (GNOS-PV02) generated for PT2. ELISpot responses were detected to the vaccine epitopes relative to background (DMSO) or screening/Day 0. Responses were also detected against the CTNNB1 mutation. Intermediate immune responses were detected against other neoepitopes included in the vaccine including INVS or SMARCA4.

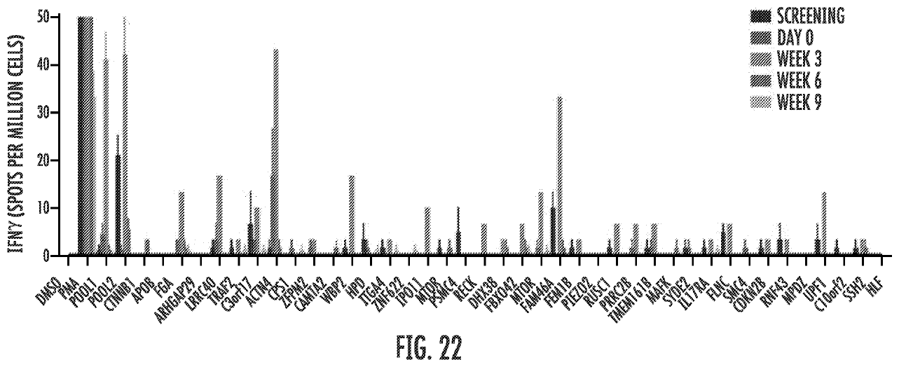

FIG. 22 shows IFNg ELISpot responses to epitopes used in the neoantigen vaccine (GNOS-PV02) generated for PT4. ELISpot responses were detected to the vaccine epitopes relative to background (DMSO) or screening/Day 0.

### Flow Cytometry Data—Patient PT6

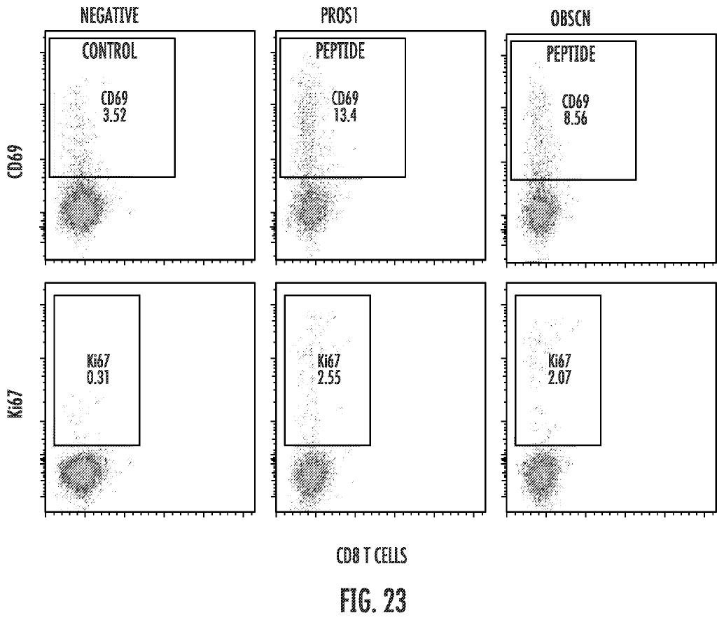

FIG. 23 shows strong CD8 T-cell responses elicited by specific epitopes encoded in GNOS-PV02 for subject PT6. T-cell activation was measured by flow cytometry (CD69 and Ki67) in peripheral blood mononuclear cells (PBMCs) 12 weeks after vaccination of subject PT6. PBMCs were stimulated with vaccine encoded neoepitope derived peptides or DMSO vehicle (negative control) in combination with IL2, IL7 and IL4 for 4 days. Following incubation, cells were washed and PBMCs were restimulated with peptides (PROS1 or OBSCN) or controls for 6 hours. Activation markers including Ki67 and CD69 were measured.

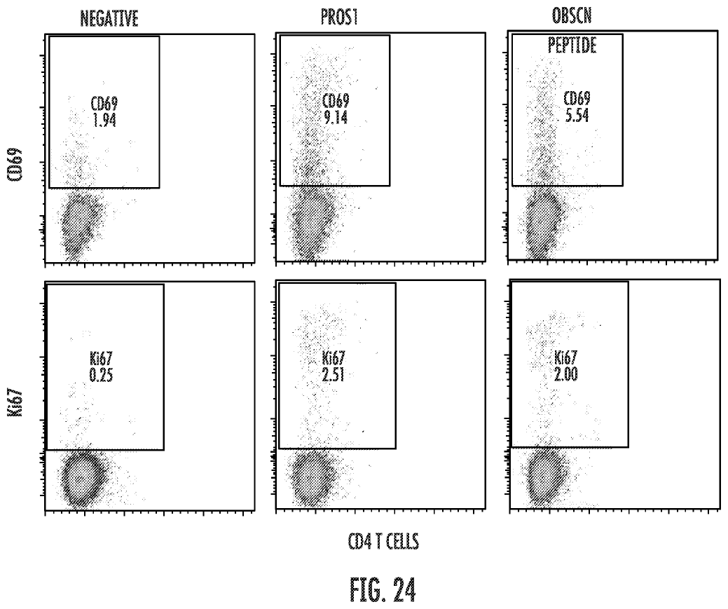

FIG. 24 shows strong CD4 T-cell responses elicited by specific epitopes encoded in GNOS-PV02 for subject PT6. T-cell activation was measured by flow cytometry (CD69 and Ki6) in PBMCs 12 weeks after vaccination of subject PT6. PBMCs were stimulated with vaccine encoded neoepitope derived peptides or DMSO vehicle (negative control) in combination with IL2, IL7 and IL4 for 4 days. Following incubation cells were washed and PBMCs were restimulated with peptides (PROS1 or OBSCN) or controls for 6 hours. Activation markers including Ki67 and CD69 were measured.

### Example 4

GNOS-PV02 Encoding Greater than 20 Neoantigens Drives Stronger and Longer Clinical Responses in Patients with and without Wnt Activating Mutations

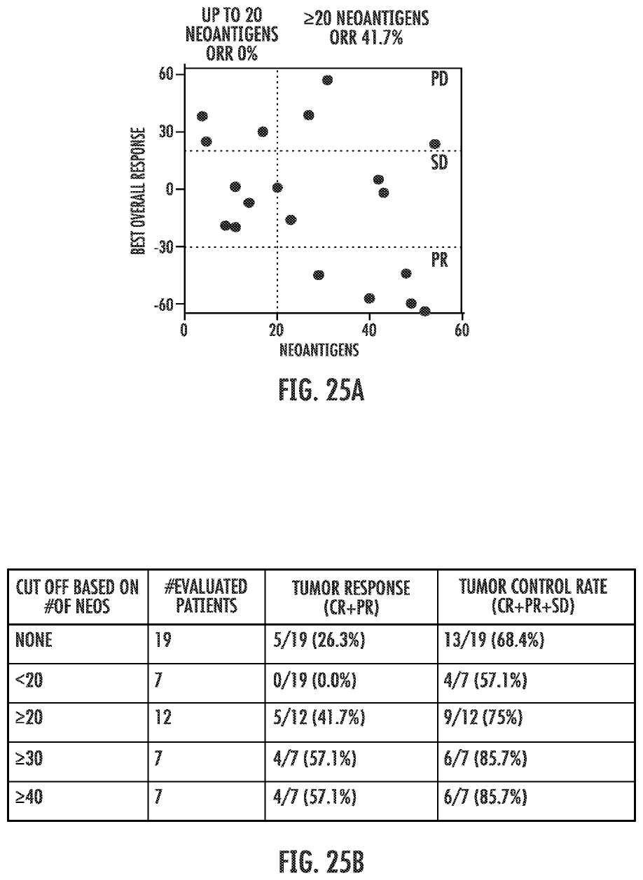

Advanced (unresectable) HCC patients were treated with GNOS-PV02 in combination with INO-9012 [encodes IL12] and pembrolizumab in second line treatment following first line treatment with lenvatinib or sorafenib. Each patient received a personalized plasmid encoding for a patient specific number of tumor neoantigens identified from their tumors. This is a patient population that is well recognized in the literature to have a low tumor mutational burden and does not respond to immune checkpoint inhibitor therapies such as anti-PD1 treatment. Published data from different CPIs such as pembrolizumab, nivolumab, tislelizumab, and sintilimab indicate that significant tumor reduction (>30%) is only observed in 12%-17% of the patients receiving such therapy. FIGS. 25A and 25B show clinical response data from 19 patients who were treated with GNOS-PV02 in combination with INO-9012 and pembrolizumab. In contrast to the pembrolizumab alone published data, GNOS-PV02 combination treatment increased the observed response rate (patients with tumor reduction >30%) to 26.3% and tumor control rate (patients whose tumors either reduced or remained stable; i.e patients whos tumors were controlled from increasing in size) to 68.4%. FIGS. 25A and 25B also shows patient responses stratified by the number of neoantigens encoded by their personalized cancer vaccine. As is clearly evident, patients who were treated with >20 neoantigens responded significantly higher rates compared to patients treated with less than 20 neoantigens. Tumor response rate of 41.7% versus 0.0% respectively, and tumor control rate of 75.0% versus 57.1% respectively. The tumor response rate and tumor control rate further increased in patients treated with greater than 30 neoantigens to 57.1% and 85.7% respectively. Thus it is evident from the clinical data that treating patients with more than 20 neoantigens conferred a benefit to the patients compared to patients treated without the patient specific personalized cancer vaccine (PCV) (i.e. with anti-PD1 therapy alone) or with a PCV encoding less than 20 neoantigens.

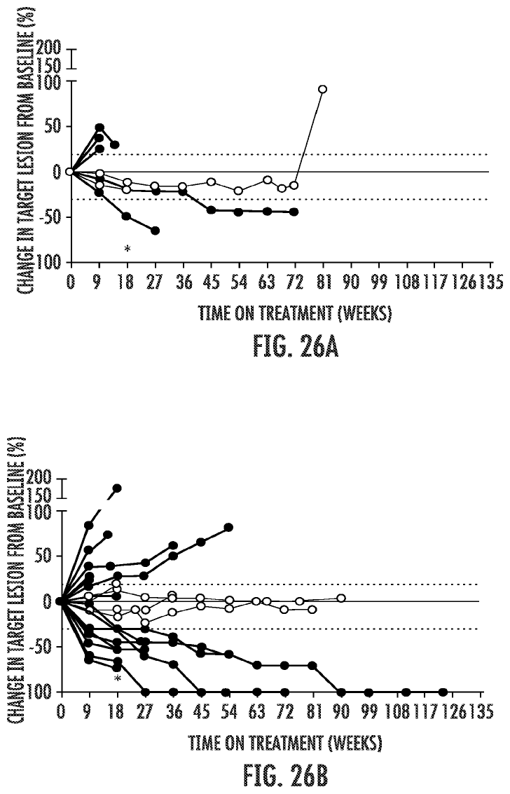

FIG. 26 shows the % change in tumor size over time for each patient. FIG. 26A shows patients treated with <20 neoantigens and FIG. 26B shows patients treated with >20 neoantigens. Similar to the overall tumor size reduction data, patients treated with greater than 20 neoantigens tended to have their tumors remain stable or continue to decrease over a longer period of time. These patients continued to be treated over an extended period of time and continue to benefit from the treatment.

Thus, it is evident from the clinical data that while all patients benefitted from the combination of GNOS-PV02 with pembrolizumab, the benefit was significantly greater in patients treated with 20 or more neoantigens.

### Example 5

### Background

Tumor neoantigens are epitopes derived from tumor-specific mutations that can be incorporated in personalized vaccines to prime T cell responses. DNA vaccines delivered with electroporation have recently shown strong CD8 and CD4 T cell responses in clinical trials. In preclinical studies, DNA-encoded neoantigen vaccines have shown induction of CD8 T cells against 50% of predicted high affinity epitopes with the ability to impact tumor growth.

### Methods

Paired blood and tumor biopsy samples were collected from a patient with hepatocellular carcinoma before and after treatment with GNOS-PV02 (DNA neoantigen targeted vaccine)+plasmid IL-12+pembrolizumab. Treatment resulted in a partial response with a decrease in tumor size of 44% by RECIST (168 mm to 94 mm). TCRbeta sequencing was performed on all 4 samples and single cell TCR and transcriptome sequencing was performed from T cells isolated from the post-treatment blood sample. Newly identified TCRs in blood and tumor after vaccination were inserted into an expression vector and used to generate engineered TCR T cells. Engineered TCR T cells were tested against the neoantigens included in the vaccine and their responses characterized by flow cytometry.

### Results

67,893 new clones were identified in PBMC after vaccination, 3 of which comprised between 0.1 to 1% of the total T cell clones. Moreover, 5126 new clones were identified in the tumor post vaccination, out of these, 3878 (75.68%) were not found within the patient's pre vaccination PBMCs and 556 (10.86%) were identified within the pre vaccination PBMC pool. Importantly, of the newly identified T cells infiltrating the tumor post vaccination, high frequency TCR clones were observed, of which 44 and 7 clones were above 0.1% and 1%, respectively. The majority of the newly identified T cell clones were CD8 T cells (68.75%) with an activated phenotype. Importantly, the 6 most expanded clones in blood were identified to be activated CD8+CD69+ T cells (81.82%). Engineered TCR T cells generated encoding the TCRs of these newly identified CD8 T cells showed activation when exposed to the tumor neoantigens encoded in the neoantigen DNA vaccine GNOS-PV02.

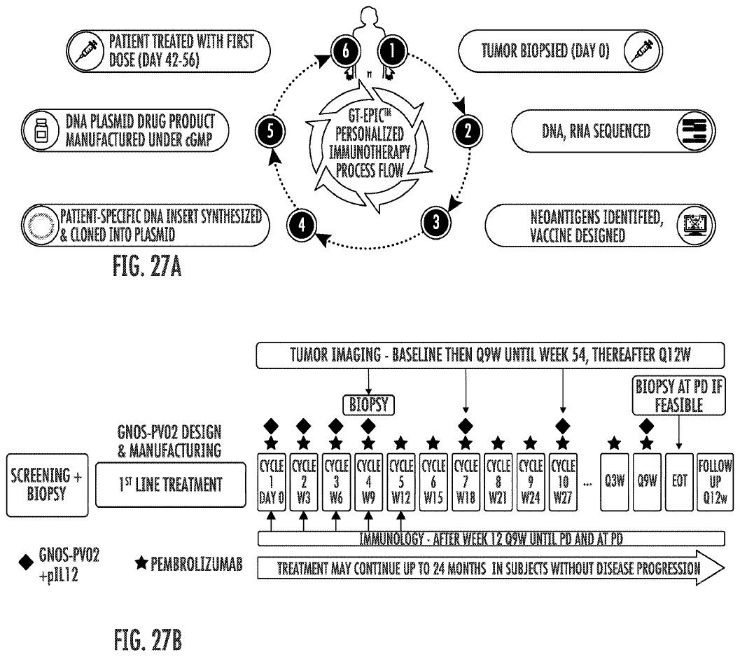

Personalized vaccines can be manufactured in 6-8 weeks allowing concurrent start with anti-PD1. FIG. 27 shows and example of the steps involved in a personalized vaccine.

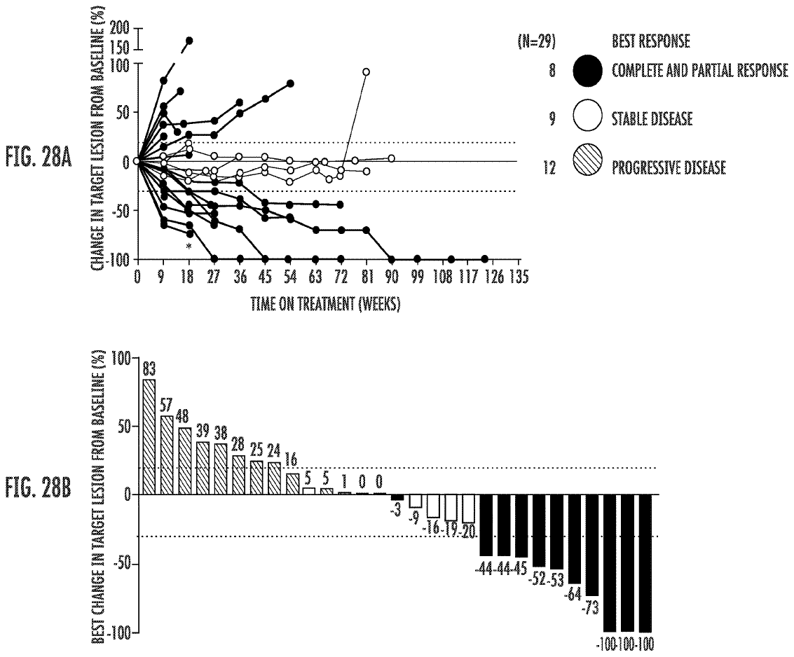

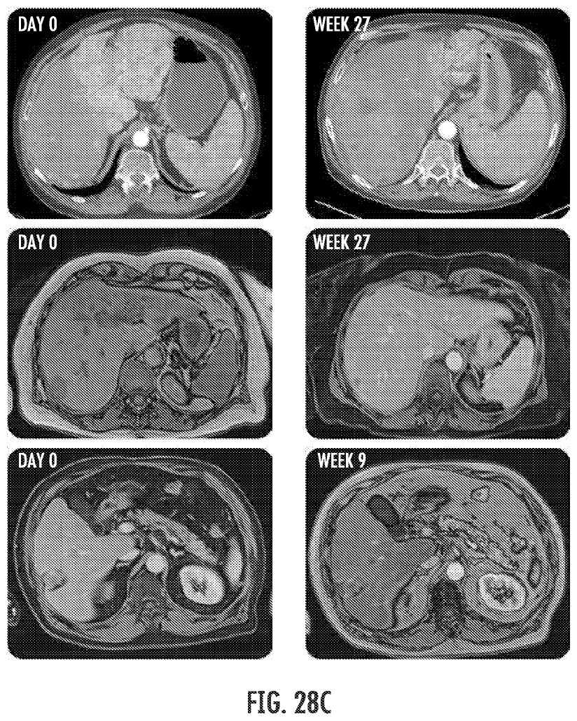

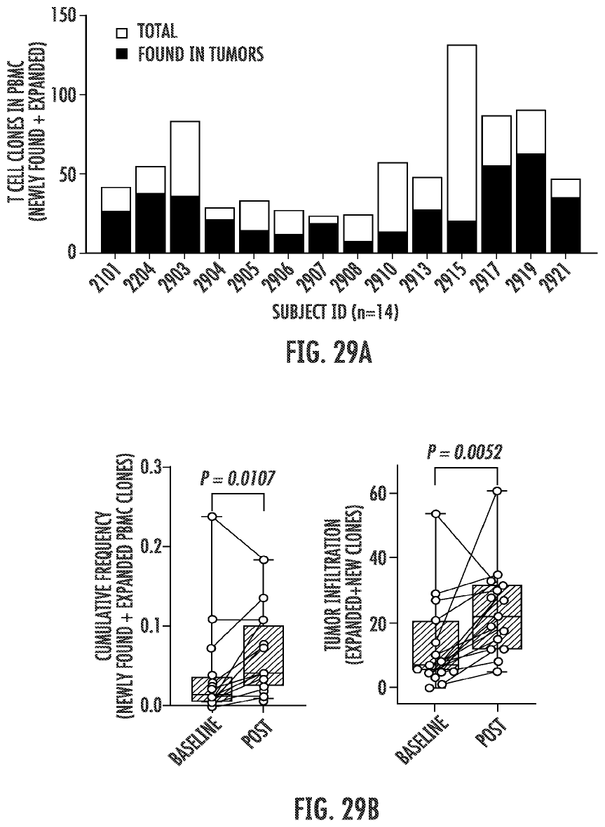

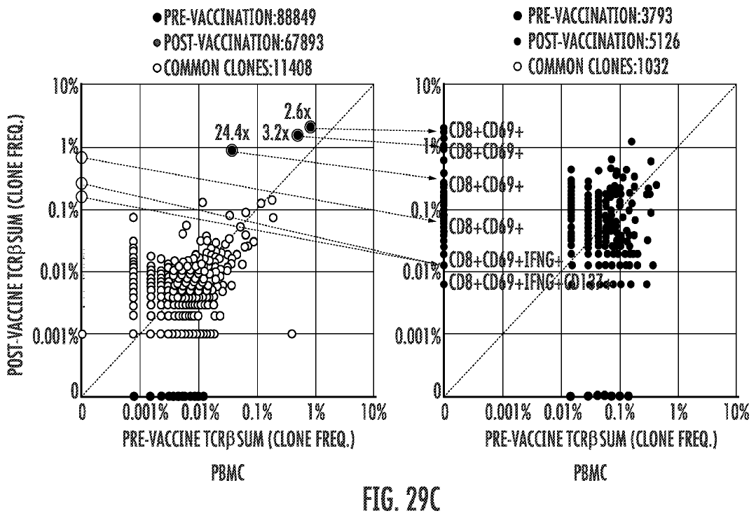

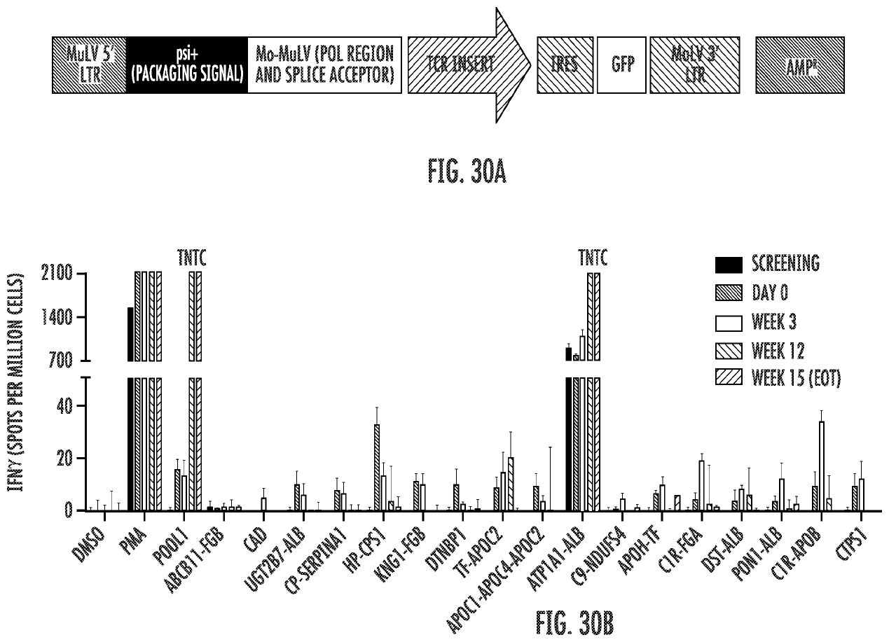

FIG. 28 shows a combination of GNOS-PV02 and anti-PD1 resulted in a 25% ORR in the first 12 patients. GNOS-PV02 results in expansion of new T cells that traffic to the tumor (see FIG. 29). As seen in FIG. 29A, post-treatment tumor samples contain significantly more of these clones than screen tumor samples (p=0.006, paired Wilcox test). FIG. 29B shows post-vaccination expansion of new T cell clones in the PBMC and their infiltration into the tumor in 9 out of 10 subjects. FIG. 29C shows that the most abundant clones show an active phenotype (CD8+CD69+) as assessed by TCRβ and RNA sequencing. Approx. 75% of new TIL clones were undetectable in blood prior to vaccination.

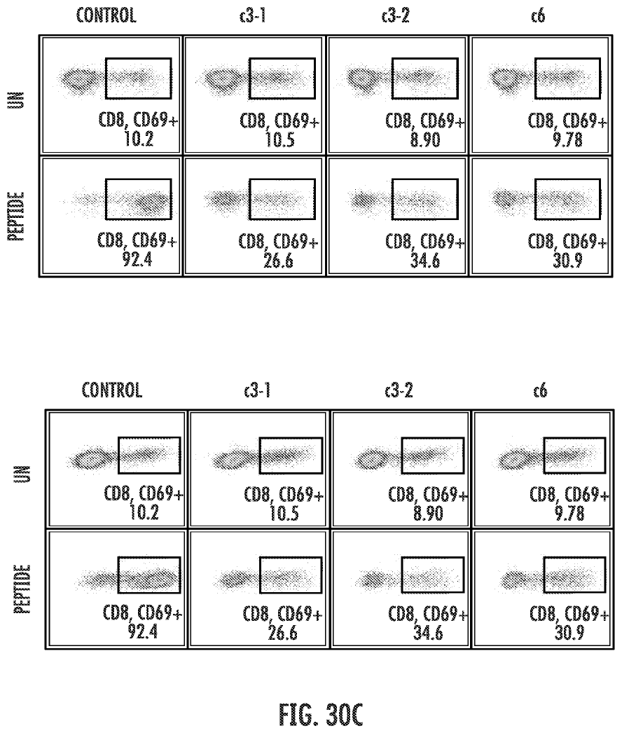

FIG. 30 shows that GNOS-PV02 generates neoantigen-specific, CD8+ and CD4+ anti-tumor responses. Table 18 shows the most frequent TCRs identified by TCRβ and RNA sequencing in subjects Pt 8 and Pt 7 on week 9 post-vaccination

TABLE 18
TCR sequences

| Construct | | | |
|---|---|---|---|
| ID | TRA (aa) | TRB (aa) | Freq. |
| 2905_c3-1 | CAVRDKGSARQLTF (SEQ | CSARDLYRNTEAFF (SEQ | 1.59E−02 |
| | ID NO: 287) | ID NO: 288) | |
| 2905_c3-2 | CAVSAGNNNARLMF | CSARDLYRNTEAFF (SEQ | 1.59E−02 |
| | (SEQ ID NO: 289) | ID NO: 290) | |
| 2905_c6 | CALDSSASKIIF (SEQ ID | CASSRQGQEQYF (SEQ | 3.72E−02 |
| | NO: 291) | ID NO: 292) | |
| 2101_c01 | CAEKGAYGQNFVF (SEQ ID | CASSSDALAGGIHRKLFF | 2.11E−02 |
| | NO: 293) | (SEQ ID NO: 294) | |
| 2101_c02 | CAFNAGNNRKLIW (SEQ ID | CASSSFRYNQPQHF (SEQ | 2.25E−04 |
| | NO: 295) | ID NO: 296) | |
| 2101_c03 | CAGVFEARLMF (SEQ ID | CASSLLQGTTAFF (SEQ | 1.86E−04 |
| | NO: 297) | ID NO: 298) | |
| 2101_c04 | CAVEDQGGKLIF (SEQ ID | CASSQDFAGSEQYF (SEQ | 1.37E−04 |
| | NO: 299) | ID NO: 300) | |
| 2101_c05 | CAVNDPPVDSSYKLIF (SEQ | CASSFADTQYF (SEQ ID | 1.27E−04 |
| | ID NO: 301) | NO: 302) | |
| 2101_c06 | CAVNDPPVDSSYKLIF (SEQ | CASSFADTQYF (SEQ ID | 1.27E−04 |
| | ID NO: 303) | NO: 304) | |
| 2101_c07 | CALAGGSYIPTF (SEQ ID | CASADSYEQYF (SEQ ID | 1.17E−04 |
| | NO: 305) | NO: 306) | |
| 2101_c08 | CAFGDSWGKFQF (SEQ ID | CASSIDQTGITEAFF (SEQ | 1.08E−04 |
| | NO: 307) | ID NO: 308) | |
| 2101_c09 | CAAIRSGSARQLTF (SEQ ID | CASSQDLGVAYNEQFF | 1.08E−04 |
| | NO: 309) | (SEQ ID NO: 310) | |
| 2101_c10 | CAYSGSARQLTF (SEQ ID | CASSFGGNTETQYF | 1.08E−04 |
| | NO: 311) | (SEQ ID NO: 312) | |
| 2101_c11 | CGTEAIGGTYKYIF (SEQ ID | CASSFGGNTETQYF | 1.08E−04 |
| | NO: 313) | (SEQ ID NO: 314) | |
| 2101_c12 | CIDSGGSNYKLTF (SEQ ID | CASSPAGPAQYF (SEQ ID | 1.57E−02 |
| | NO: 315) | NO: 316) | |
| 2101_c13 | CAGRLSSGGSYIPTF (SEQ | CASSAIGTPSGEQFF | 9.01E−03 |
| | ID NO: 317) | (SEQ ID NO: 318) | |
| 2101_c14 | CAVGSDGAGNMLTF (SEQ | CASTRDESYNSPLHF | 6.94E−03 |
| | ID NO: 319) | (SEQ ID NO: 320) | |
| 2101_c15 | CAVGGRSNAGGTSYGKLTF | CASTRDESYNSPLHF | 6.94E−03 |
| | (SEQ ID NO: 321) | (SEQ ID NO: 322) | |
| 2101_c16 | CAASVGGAGANNLFF (SEQ | CASSVDIYNEQFF (SEQ | 2.64E−03 |
| | ID NO: 323) | ID NO: 324) | |
| 2101_c17 | CAMREPYNFNKFYF (SEQ | CATHEGWDTGELFF | 1.66E−03 |
| | ID NO: 325) | (SEQ ID NO: 326) | |
| 2101_c18 | CAAGGTSYGKLTF (SEQ ID | CATTSGSPAGELFF (SEQ | 1.42E−03 |
| | NO: 327) | ID NO: 328) | |
| 2101_c19 | CAAETRNNNARLMF (SEQ | CAGRLAGASGELFF | 1.27E−03 |
| | ID NO: 329) | (SEQ ID NO: 330) | |
| 2101_c20 | CAFLLSDGQKLLF (SEQ ID | CAGRLAGASGELFF | 1.27E−03 |
| | NO: 331) | (SEQ ID NO: 332) | |
| 2101_c21 | CAAAGYSSASKIIF (SEQ ID | CASSLGPPGQTEAFF | 7.43E−04 |
| | NO: 333) | (SEQ ID NO: 334) | |
| 2101_c22 | CAMSAPWDTGNQFYF | CASSHGQGKDGELFF | 7.43E−04 |
| | (SEQ ID NO: 335) | (SEQ ID NO: 336) | |
| 2101_c23 | CAAEGNTDKLIF (SEQ ID | CASSQDPAGRWTEAFF | 7.34E−04 |
| | NO: 337) | (SEQ ID NO: 338) | |
| 2101_c24 | CTTSGTYKYIF (SEQ ID | CASSPRQGPGELFF (SEQ | 1.76E−04 |
| | NO: 339) | ID NO: 340) | |
| 2101_c25 | CAVEEISGGYQKVTF (SEQ | CASSENRGRADTQYF | 2.93E−04 |
| | ID NO: 341) | (SEQ ID NO: 342) | |
| 2101_c26 | CAVEEISGGYQKVTF (SEQ | CASSENRGRADTQYF | 2.93E−04 |
| | ID NO: 343) | (SEQ ID NO: 344) | |
| 2101_c27 | CAVEEISGGYQKVTF (SEQ | CASSENRGRADTQYF | 2.93E−04 |
| | ID NO: 345) | (SEQ ID NO: 346) | |
| 2101_c28 | CAESIGDYKLSF (SEQ ID | CASSQELVGPNTGELFF | 9.78E−05 |
| | NO: 347) | (SEQ ID NO: 348) | |
| 2101_c29 | CAATSMDSNYQLIW (SEQ | CASSIEQGVAQYF (SEQ | 1.37E−04 |
| | ID NO: 349) | ID NO: 350) | |
| 2101_c30 | CAFTYSGGGADGLTF (SEQ | CATSREGATFYNEQFF | 5.48E−04 |
| | ID NO: 351) | (SEQ ID NO: 352) | |
| 2101_c31 | CIVRVAGFLYSGGGADGLTF | CASSVGEFTGTDTQYF | 8.80E−05 |
| | (SEQ ID NO: 353) | (SEQ ID NO: 354) | |
| 2101_c32 | CVVSRGDYKLSF (SEQ ID | CAINPDSNQPQHF (SEQ | 3.13E−04 |
| | NO: 355) | ID NO: 356) | |
| 2101_c33 | CAVGNARLMF (SEQ ID | CAINPDSNQPQHF (SEQ | 3.13E−04 |
| | NO: 357) | ID NO: 358) | |
| 2101_c34 | CAVSPMTGFQKLVF (SEQ | CASSELEGDGSGNTIYF | 8.80E−05 |
| | ID NO: 359) | (SEQ ID NO: 360) | |
| 2101_c35 | CAASGTSYGKLTF (SEQ ID | CASSQGGSGTLFHEQFF | 4.01E−04 |
| | NO: 361) | (SEQ ID NO: 362) | |
| 2101_c36 | CAVPHGATNKLIF (SEQ ID | CASKGVVYEQYF (SEQ | 2.25E−04 |
| | NO: 363) | ID NO: 364) | |
| 2101_c37 | CAEIPFSGGYNKLIF (SEQ | CASSGRTGGGYEQYF | 2.15E−04 |
| | ID NO: 365) | (SEQ ID NO: 366) | |

### CONCLUSIONS

GT-EPIC™ personalized vaccines containing up to about 40 neoantigens can be designed, manufactured, and administered successfully in as short as 6 weeks allowing concurrent start with anti-PD1 in 2nd line HCC. GNOS-PV02+INO-9012 in combination with pembrolizumab achieved an ORR of 25% in the first 12 patients of the clinical trial (3 PR) and a DCR of 67%. Patients treated with GNOS-PV02+INO-9012 in combination with pembrolizumab had new T cell clones in blood following vaccination, with new clones comprising up to 1% of the peripheral T cell repertoire. Engineered TCRs identified from high-frequency T cell clones in tumor post-treatment, respond to peptides encoded in the vaccine. GNOS-PV02+INO-9012 present an unremarkable safety profile with no treatment-related SAEs.

GNOS-PV02, a neoantigen DNA vaccine, in combination with plasmid encoding IL-12 or functional fragments thereof and pembrolizumab resulted in expansion of newly identified T cells, primarily activated CD8, which trafficked to the tumor. These new tumor infiltrating T cells showed TCR specificity against tumor neoantigens encoded in GNOS-PV02 and may account for the observed objective decrease in tumor size.

1. A method of treating hepatocellular cancer (HCC) in a subject in need thereof comprising:
administering to the subject a pharmaceutical composition comprising a nucleic acid sequence encoding about 20 to about 60 neoantigens; and
administering to the subject a therapeutically effective amount of an anti-PD1 therapy.

2. The method of claim 1, wherein the pharmaceutical composition comprises a plasmid comprising the nucleic acid sequence and the nucleic acid sequence encodes from about 20 to about 40 neoantigens.

3. The method of claim 2, wherein the anti-PD1 therapy comprises Pembrolizumab, Nivolumab, Cemiplimab, Dostarlimab, Atezolizumab, Avelumab, Durvalumab, Ipilimumab, Relatlimab, Spartalizumab, Camrelizumab, Sintilimab, Tislelizumab, Toripalimab, Tremelimumab, or a combination thereof.

4. The method of claim 3, wherein the anti-PD1 therapy comprises Pembrolizumab.

5. The method of claim 3, wherein the anti-PD1 therapy comprises Nivolumab.

6. The method of claim 3, wherein the anti-PD1 therapy comprises Cemiplimab.

7. The method of claim 3, wherein the anti-PD1 therapy comprises Dostarlimab.

8. The method of claim 4, wherein the HCC is a WNT activated HCC.

9. The method of claim 8, wherein the nucleic acid sequence encodes independently selected, non-repetitive antigen expression domains (AEDs) according to Formula I: [(AEDⁿ)-(linker)]ₙ-[AEDⁿ⁺¹], wherein each AED encodes one of the neoantigens, and wherein each linker is a nucleic acid sequence encoding one or a plurality of amino acid cleavage sites.

10. The method of claim 9, wherein each linker has a length independently selectable from about 15 to about 300 nucleotides and encodes a furin cleavage site.

11. The method of claim 10, wherein the length of each AED is independently selectable from about 12 to about 15,000 nucleotides.

12. The method of claim 11, wherein n is any positive integer from about 19 to about 58.

13. The method of claim 8, wherein the administering is intradermal or intramuscular by electroporation.

14. The method of claim 13, further comprising co-administering a nucleic acid encoding IL-12.

15. The method of claim 14, wherein the subject has HCC that has not responded to the anti-PD1 therapy.

16. The method of claim 15, wherein the method achieves an objective response rate (ORR) of about 50% in WNT activated HCC patients.
